(19) United States
(12) Patent Application Publication
(10) Pub. No.: US 2023/0293685 A1
(43) Pub. Date: Sep. 21, 2023

(54) SELECTION OF IMPROVED TUMOR REACTIVE T-CELLS

(71) Applicant: Iovance Biotherapeutics, Inc., San Carlos, CA (US)

(72) Inventors: Michelle Simpson-Abelson, Lithia, FL (US); Arvind Natarajan, Basking Ridge, NJ (US)

(21) Appl. No.: 17/997,648

(22) PCT Filed: May 4, 2021
(86) PCT No.: PCT/US21/30655

§ 371 Date: Oct. 31, 2022

Related U.S. Application Data
(60) Provisional application No. 63/146,400, filed on Feb. 5, 2021.
(60) Provisional application No. 63/019,907, filed on May 4, 2020.

(51) Int. Cl. A61K 39/00; C07K 14/705; C07K 14/725; C07K 14/55; C07K 16/28; A61K 31/664; A61K 31/7076; C12N 5/0783

(52) U.S. Cl. CPC A61K 39/4611; C07K 14/70521; C07K 14/70596; C07K 14/70546; C07K 14/70503; C07K 14/7051; C07K 14/55; C07K 16/2818; C07K 14/70517; A61K 39/4633; A61K 39/464429; A61K 39/464411; A61K 39/4632; A61K 39/4635; A61K 39/4622; A61K 39/4614; A61K 31/664; A61K 31/7076; C12N 5/0636; C07K 2317/21; C07K 2317/14; C07K 2317/52; C07K 2317/24; C12N 2501/2302

(57) ABSTRACT

The present invention provides methods for preselecting TILs based on PD-1, CD39, CD38, CD103, CD101, LAG3, TIM3 and/or TIGIT expression, as well as methods for expanding those preselected PD-1, CD39, CD38, CD103, CD101, LAG3, TIM3 and/or TIGIT positive TILs in order to produce therapeutic populations of TILs with enhanced tumor-specific killing capacity (e.g., enhanced cytotoxicity).

### CROSS-REFERENCE TO RELATED APPLICATIONS

The present application claims priority to U.S. Provisional Patent Application No. 63/019,907, filed on May 4, 2020, and U.S. Provisional Patent Application No. 63/146,400, filed Feb. 5, 2021, the disclosures of which are hereby incorporated by reference in their entireties for all purposes.

### BACKGROUND OF THE INVENTION

Treatment of bulky, refractory cancers using adoptive transfer of tumor infiltrating lymphocytes (TILs) represents a powerful approach to therapy for patients with poor prognoses. Gattinoni, et al., Nat. Rev. Immunol. 2006, 6, 383-393. A large number of TILs are required for successful immunotherapy, and a robust and reliable process is needed for commercialization. This has been a challenge to achieve because of technical, logistical, and regulatory issues with cell expansion. IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J Immunother. 2003, 26, 332-42. REP can result in a 1,000-fold expansion of TILs over a 14-day period, although it requires a large excess (e.g., 200-fold) of irradiated allogeneic peripheral blood mononuclear cells (PBMCs, also known as mononuclear cells (MNCs)), often from multiple donors, as feeder cells, as well as anti-CD3 antibody (OKT3) and high doses of IL-2. Dudley, et al., J. Immunother. 2003, 26, 332-42. TILs that have undergone an REP procedure have produced successful adoptive cell therapy following host immunosuppression in patients with melanoma. Current infusion acceptance parameters rely on readouts of the composition of TILs (e.g., CD28, CD8, or CD4 positivity) and on fold expansion and viability of the REP product.

Current TIL manufacturing processes are limited by length, cost, sterility concerns, and other factors described herein such that the potential to commercialize such processes is severely limited. While there has been characterization of TILs, for example, TILs have been shown to express various receptors, including inhibitory receptors programmed cell death 1 (PD-1; also known as CD279) (see, Gros, A., et al., Clin Invest. 124(5):2246-2259 (2014)), the usefulness of this information in developing therapeutic TIL populations has yet to be fully realized. There is an urgent need to provide TIL manufacturing processes and therapies based on such processes that are appropriate for commercial scale manufacturing and regulatory approval for use in human patients at multiple clinical centers. The present invention meets this need by providing methods for preselecting TILs based on PD-1, CD39, CD38, CD103, CD101, LAG3, TIM3 and/or TIGIT expression in order to obtain TILs with enhanced tumor-specific killing capacity (e.g., enhanced cytotoxicity).

### BRIEF SUMMARY OF THE INVENTION

The present invention provides methods for expanding TILs and producing therapeutic populations of TILs, which includes a PD-1+, CD39+, CD103+, CD101+, LAG3+, TIM3+ and/or TIGIT+ status preselection step.

In some embodiments, the present provides a method for expanding tumor infiltrating lymphocytes (TILs) into a therapeutic population of TILs comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs;
(d) performing a rapid second expansion by supplementing the cell culture medium of the second population of TILs with additional IL-2, OKT-3, and APCs, to produce a third population of TILs, wherein the number of APCs added in the rapid second expansion is at least twice the number of APCs added in step (c), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(e) harvesting therapeutic population of TILs obtained from step (d); and
(f) transferring the harvested TIL population from step (e) to an infusion bag.

In some embodiments, the present provides a method for expanding tumor infiltrating lymphocytes (TILs) into a therapeutic population of TILs comprising:
a) obtaining and/or receiving a first population of TILs from a tumor resected from a subject by processing a tumor sample obtained from the subject into multiple tumor fragments;
b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
c) performing a priming first expansion by culturing the PD-1, CD39, CD38, CD103,
CD101, LAG3, TIM3 and/or TIGIT enriched TIL population in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs;
d) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and
e) harvesting therapeutic population of TILs obtained from step (d).

In some embodiments, in step (c) the cell culture medium further comprises antigen-presenting cells (APCs), and wherein the number of APCs in the culture medium in step (d) is greater than the number of APCs in the culture medium in step (c).

In some embodiments, in step (c) the cell culture medium further comprises antigen-presenting cells (APCs), and wherein the number of APCs in the culture medium in step (d) is equal to the number of APCs in the culture medium in step (c).

In some embodiments, the PD-1, CD39, CD38, CD103, CD101, LAG3, TIM3 and/or TIGIT positive TILs are PD-1high, CD39high/lo, CD38lo; CD103high/lo, CD101lo, LAG3high, TIM3high and/or TIGIThigh TILs.

In some embodiments, the present provides a method for expanding tumor infiltrating lymphocytes (TILs) into a therapeutic population of TILs comprising:
(a) performing a priming first expansion by culturing a first population of TILs which have been selected to be PD-1, CD39, CD38, CD103, CD101, LAG3, TIM3 and/or TIGIT positive, said first population of TILs obtainable by processing a tumor sample from a subject by tumor digestion and selecting for the PD-1, CD39, CD38, CD103, CD101, LAG3, TIM3 and/or TIGIT positive TILs, in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs;
(b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area; and
(c) harvesting therapeutic population of TILs obtained from step (b).

In some embodiments, the present provides a method for expanding tumor infiltrating lymphocytes (TILs) into a therapeutic population of TILs comprising:
(a) performing a priming first expansion of a first population of TILs which have been selected to be PD-1, CD39, CD38, CD103, CD101, LAG3, TIM3 and/or TIGIT positive by culturing the first population of TILs in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs;
(b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and
(c) harvesting therapeutic population of TILs obtained from step (b).

In some embodiments, in step (a) the cell culture medium further comprises antigen-presenting cells (APCs), and wherein the number of APCs in the culture medium in step (b) is greater than the number of APCs in the culture medium in step (a).

In some embodiments, in step (a) the cell culture medium further comprises antigen-presenting cells (APCs), and wherein the number of APCs in the culture medium in step (b) is the equal to the number of APCs in the culture medium in step (a).

In some embodiments, the PD-1, CD39, CD38, CD103, CD101, LAG3, TIM3 and/or TIGIT positive TILs are PD-1high, CD39high, CD38lo, CD103high, CD101lo, LAG3high, TIM3high and/or TIGIThigh TILs.

In some embodiments, the selection of step (b) or the selection of step (a), comprises a selection method selected from the group consisting of flow cytometry (including for example FACS), antibody-based bead selection, and antibody-based magnetic bead selection.

In some embodiments, the selection of step (b) or the selection of step (a), comprises flow cytometry (including for example FACS).

In some embodiments, the selection of step (b) or the selection of step (a), comprises the steps of (i) exposing the first population of TILs to an excess of a monoclonal anti-PD-1 IgG4 antibody that binds to PD-1 through an N-terminal loop outside the IgV domain of PD-1, (ii) adding an excess of an anti-IgG4 antibody conjugated to a fluorophore, and (iii) performing a flow-based cell sort based on the fluorophore to obtain a PD-1 enriched TIL population.

In some embodiments, the selection of PD-1, CD39, CD38, CD103, CD101, LAG3, TIM3 and/or TIGIT positive TILs occurs until there are at least 1×106 PD-1, CD39, CD38, CD103, CD101, LAG3, TIM3 and/or TIGIT positive TILs.

In some embodiments, the cell culture medium for culturing the first population of TILs comprises 2-mercaptoethanol.

In some embodiments, the cell culture medium for culturing the second population of TILs comprises 2-mercaptoethanol.

In some embodiments, the cell culture medium for culturing the first population of TILs and the second population of TILs comprises 2-mercaptoethanol.

In some embodiments, the PD-1, CD39, CD38, CD103, CD101, LAG3, TIM3 and/or TIGIT positive TILs are selected using an anti-PD-1, anti-CD39, anti-CD38, anti-CD103, anti-CD101, anti-LAG3, anti-TIM3 and/or anti-TIGIT antibody conjugated bead, respectively.

In some embodiments, the PD-1, CD39, CD38, CD103, CD101, LAG3, TIM3 and/or TIGIT positive TILs are selected using an anti-PD-1, anti-CD39, anti-CD38, anti-CD103, anti-CD101, anti-LAG3, anti-TIM3 and/or anti-TIGIT antibody conjugated magnetic bead, respectively.

In some embodiments, the PD-1, CD39, CD38, CD103, CD101, LAG3, TIM3 and/or TIGIT positive TILs bind to an anti-PD-1, anti-CD39, anti-CD38, anti-CD103, anti-CD101, anti-LAG3, anti-TIM3 and/or anti-TIGIT antibody conjugated beads, respectively, and the PD-1, CD39, CD38, CD103, CD101, LAG3, TIM3 and/or TIGIT negative TILs do not bind to an anti-PD-1, anti-CD39, anti-CD38, anti-CD103, anti-CD101, anti-LAG3, anti-TIM3 and/or anti-TIGIT antibody conjugated bead, respectively.

In some embodiments, the monoclonal anti-PD-1 IgG4 antibody is nivolumab or variants, fragments, or conjugates thereof

In some embodiments, the anti-IgG4 antibody is clone anti-human IgG4, Clone HP6023.

In some embodiments, the number of APCs in the rapid second expansion to the number of APCs in the priming first expansion is a ratio selected from a range of from about 1.5:1 to about 20:1.

In some embodiments, the ratio is selected from a range of from about 1.5:1 to about 10:1.

In some embodiments, the ratio is selected from a range of from about 2:1 to about 5:1.

In some embodiments, the ratio is selected from a range of from about 2:1 to about 3:1.

In some embodiments, the ratio is about 2:1.

In some embodiments, the number of APCs in the priming first expansion is selected from the range of about 1×10⁸ APCs to about 3.5×10⁸ APCs, and wherein the number of APCs in the rapid second expansion is selected from the range of about 3.5×10⁸ APCs to about 1×10⁹ APCs.

In some embodiments, the number of APCs in the priming first expansion is selected from the range of about 1.5×10⁸ APCs to about 3×10⁸ APCs, and wherein the number of APCs in the rapid second expansion is selected from the range of about 4×10⁸ APCs to about 7.5×10⁸ APCs.

In some embodiments, the number of APCs in the priming first expansion is selected from the range of about 2×10⁸ APCs to about 2.5×10⁸ APCs, and wherein the number of APCs in the rapid second expansion is selected from the range of about 4.5×10⁸ APCs to about 5.5×10⁸ APCs.

In some embodiments, about 2.5×10⁸ APCs are added to the priming first expansion and 5×10⁸ APCs are added to the rapid second expansion.

In some embodiments, the ratio of the number of TILs in the second population of TILs to the number of TILs in the first population of TILs is about 1.5:1 to about 100:1.

In some embodiments, the ratio of the number of TILs in the second population of TILs to the number of TILs in the first population of TILs is about 50:1.

In some embodiments, the ratio of the number of TILs in the second population of TILs to the number of TILs in the first population of TILs is about 25:1.

In some embodiments, the ratio of the number of TILs in the second population of TILs to the number of TILs in the first population of TILs is about 20:1.

In some embodiments, the ratio of the number of TILs in the second population of TILs to the number of TILs in the first population of TILs is about 10:1.

In some embodiments, the second population of TILs is at least 50-fold greater in number than the first population of TILs.

In some embodiments, the method comprises performing, after the step of harvesting the therapeutic population of TILs, the additional step of:

transferring the harvested therapeutic population of TILs to an infusion bag.

In some embodiments, the priming first expansion is performed in a plurality of separate containers, in each of which separate containers the second population of TILs is obtained from the first population of TILs in the step of the priming first expansion, and the third population of TILs is obtained from the second population of TILs in the step of the rapid second expansion, and wherein therapeutic population of TILs obtained from the third population of TILs is collected from each of the plurality of containers and combined to yield the harvested TIL population.

In some embodiments, the plurality of separate containers comprises at least two separate containers.

In some embodiments, the plurality of separate containers comprises from two to twenty separate containers.

In some embodiments, the plurality of separate containers comprises from two to ten separate containers.

In some embodiments, the plurality of separate containers comprises from two to five separate containers.

In some embodiments, each of the separate containers comprises a first gas-permeable surface area.

In some embodiments, the priming first expansion step is performed in a single container.

In some embodiments, the single container comprises a first gas-permeable surface area.

In some embodiments, in the step of the priming first expansion the cell culture medium comprises antigen-presenting cells (APCs) and the APCs are layered onto the first gas-permeable surface area at an average thickness of about one cell layer to about three cell layers.

In some embodiments, in the step of the priming first expansion the APCs are layered onto the first gas-permeable surface area at an average thickness of about 1.5 cell layers to about 2.5 cell layers.

In some embodiments, n the step of the priming first expansion the APCs are layered onto the first gas-permeable surface area at an average thickness of about 2 cell layers.

In some embodiments, in the step of the rapid second expansion the APCs are layered onto the first gas-permeable surface area at a thickness of about 3 cell layers to about 5 cell layers.

In some embodiments, in the step of the rapid second expansion the APCs are layered onto the first gas-permeable surface area at a thickness of about 3.5 cell layers to about 4.5 cell layers.

In some embodiments, in the step of the rapid second expansion the APCs are layered onto the first gas-permeable surface area at a thickness of about 4 cell layers.

In some embodiments, in the step of the priming first expansion, the priming first expansion is performed in a first container comprising a first gas-permeable surface area, and in the step of the rapid second expansion, the rapid second expansion is performed in a second container comprising a second gas-permeable surface area.

In some embodiments, the second container is larger than the first container.

In some embodiments, in the step of the priming first expansion the cell culture medium comprises antigen-presenting cells (APCs) and the APCs are layered onto the first gas-permeable surface area at an average thickness of about one cell layer to about three cell layers.

In some embodiments, in the step of the priming first expansion the APCs are layered onto the first gas-permeable surface area at an average thickness of about 1.5 cell layers to about 2.5 cell layers.

In some embodiments, in the step of the priming first expansion the APCs are layered onto the first gas-permeable surface area at an average thickness of about 2 cell layers.

In some embodiments, in the step of the rapid second expansion the APCs are layered onto the second gas-permeable surface area at an average thickness of about 3 cell layers to about 5 cell layers.

In some embodiments, in the step of the rapid second expansion the APCs are layered onto the second gas-permeable surface area at an average thickness of about 3.5 cell layers to about 4.5 cell layers.

In some embodiments, in the step of the rapid second expansion the APCs are layered onto the second gas-permeable surface area at an average thickness of about 4 cell layers.

In some embodiments, for each container in which the priming first expansion is performed on a first population of TILs the rapid second expansion is performed in the same container on the second population of TILs produced from such first population of TILs.

In some embodiments, each container comprises a first gas-permeable surface area.

In some embodiments, in the step of the priming first expansion the cell culture medium comprises antigen-presenting cells (APCs) and the APCs are layered onto the first gas-permeable surface area at an average thickness of from about one cell layer to about three cell layers.

In some embodiments, in the step of the priming first expansion the APCs are layered onto the first gas-permeable surface area at an average thickness of from about 1.5 cell layers to about 2.5 cell layers.

In some embodiments, in the step of the priming first expansion the APCs are layered onto the first gas-permeable surface area at an average thickness of about 2 cell layers.

In some embodiments, in the step of the rapid second expansion the APCs are layered onto the first gas-permeable surface area at an average thickness of about 3 cell layers to about 5 cell layers.

In some embodiments, in the step of the rapid second expansion the APCs are layered onto the first gas-permeable surface area at an average thickness of about 3.5 cell layers to about 4.5 cell layers.

In some embodiments, in the step of the rapid second expansion the APCs are layered onto the first gas-permeable surface area at an average thickness of about 4 cell layers.

In some embodiments, for each container in which the priming first expansion is performed on a first population of TILs in the step of the priming first expansion the container comprises a first gas-permeable surface area, the cell culture medium comprises antigen-presenting cells (APCs), and the APCs are layered onto the first gas-permeable surface area, and wherein the ratio of the average number of layers of APCs layered in the step of the priming first expansion to the average number of layers of APCs layered in the step of the rapid second expansion is selected from the range of about 1:1.1 to about 1:10.

In some embodiments, the ratio of the average number of layers of APCs layered in the step of the priming first expansion to the average number of layers of APCs layered in the step of the rapid second expansion is selected from the range of about 1:1.2 to about 1:8.

In some embodiments, the ratio of the average number of layers of APCs layered in the step of the priming first expansion to the average number of layers of APCs layered in the step of the raid second expansion is selected from the range of about 1:1.3 to about 1:7.

In some embodiments, the ratio of the average number of layers of APCs layered in the step of the priming first expansion to the average number of layers of APCs layered in the step of the rapid second expansion is selected from the range of about 1:1.4 to about 1:6.

In some embodiments, the ratio of the average number of layers of APCs layered in the step of the priming first expansion to the average number of layers of APCs layered in the step of the rapid second expansion is selected from the range of about 1:1.5 to about 1:5.

In some embodiments, the ratio of the average number of layers of APCs layered in the step of the priming first expansion to the average number of layers of APCs layered in the step of the rapid second expansion is selected from the range of about 1:1.6 to about 1:4.

In some embodiments, the ratio of the average number of layers of APCs layered in the step of the priming first expansion to the average number of layers of APCs layered in the step of the rapid second expansion is selected from the range of about 1:1.7 to about 1:3.5.

In some embodiments, the ratio of the average number of layers of APCs layered in the step of the priming first expansion to the average number of layers of APCs layered in the step of the rapid second expansion is selected from the range of about 1:1.8 to about 1:3.

In some embodiments, the ratio of the average number of layers of APCs layered in the step of the priming first expansion to the average number of layers of APCs layered in the step of the rapid second expansion is selected from the range of about 1:1.9 to about 1:2.5.

In some embodiments, the ratio of the average number of layers of APCs layered in the step of the priming first expansion to the average number of layers of APCs layered in the step of the rapid second expansion is about 1:2.

In some embodiments, after 2 to 3 days in the step of the rapid second expansion, the cell culture medium is supplemented with additional IL-2.

In some embodiments, the method further comprises cryopreserving the harvested TIL population in the step of harvesting therapeutic population of TILs using a cryopreservation process.

In some embodiments, the method further comprises the step of cryopreserving the infusion bag.

In some embodiments, the cryopreservation process is performed using a 1:1 ratio of harvested TIL population to cryopreservation media.

In some embodiments, the antigen-presenting cells are peripheral blood mononuclear cells (PBMCs).

In some embodiments, the PBMCs are irradiated and allogeneic.

In some embodiments, in the step of the priming first expansion the cell culture medium comprises peripheral blood mononuclear cells (PBMCs), and wherein the total number of PBMCs in the cell culture medium in the step of the priming first expansion is 2.5×10⁸.

In some embodiments, in the step of the rapid second expansion the antigen-presenting cells (APCs) in the cell culture medium are peripheral blood mononuclear cells (PBMCs), and wherein the total number of PBMCs added to the cell culture medium in the step of the rapid second expansion is 5×10⁸.

In some embodiments, the antigen-presenting cells are artificial antigen-presenting cells.

In some embodiments, the harvesting in the step of harvesting therapeutic population of TILs is performed using a membrane-based cell processing system.

In some embodiments, the harvesting in step (d) is performed using a LOVO cell processing system.

In some embodiments, the multiple fragments comprise about 60 fragments per container in the step of the priming first expansion, wherein each fragment has a volume of about 27 mm³.

In some embodiments, the multiple fragments comprise about 30 to about 60 fragments with a total volume of about 1300 mm³ to about 1500 mm³.

In some embodiments, the multiple fragments comprise about 50 fragments with a total volume of about 1350 mm³.

In some embodiments, the multiple fragments comprise about 50 fragments with a total mass of about 1 gram to about 1.5 grams.

In some embodiments, the cell culture medium is provided in a container selected from the group consisting of a G-container and a Xuri cellbag.

In some embodiments, after 2 to 3 days in step (d), the cell culture medium is supplemented with additional IL-2.

In some embodiments, the IL-2 concentration is about 10,000 IU/mL to about 5,000 IU/mL.

In some embodiments, the IL-2 concentration is about 6,000 IU/mL.

In some embodiments, the infusion bag in the step of transferring the harvested therapeutic population of TILs to an infusion bag is a HypoThermosol-containing infusion bag.

In some embodiments, the cryopreservation media comprises dimethlysulfoxide (DMSO).

In some embodiments, the cryopreservation media comprises 7% to 10% DMSO.

In some embodiments, the first period in the step of the priming first expansion and the second period in the step of the rapid second expansion are each individually performed within a period of 5 days, 6 days, 7 days, 8 days, 9 days, 10 days, or 11 days.

In some embodiments, the first period in the step of the priming first expansion is performed within a period of 5 days, 6 days, or 7 days.

In some embodiments, the first period in the step of the priming first expansion is performed within a period of 8 days, 9 days, 10 days, or 11 days.

In some embodiments, the second period in the step of the rapid second expansion is performed within a period of 7 days, 8 days, or 9 days.

In some embodiments, the second period in the step of the rapid second expansion is performed within a period of 10 days or 11 days.

In some embodiments, the first period in the step of the priming first expansion and the second period in the step of the rapid second expansion are each individually performed within a period of 7 days.

In some embodiments, the first period in the step of the priming first expansion and the second period in the step of the rapid second expansion are each individually performed within a period of 11 days.

In some embodiments, steps of the priming first expansion through the harvesting of the therapeutic population of TILs are performed within a period of about 14 days to about 16 days.

In some embodiments, steps of the priming first expansion through the harvesting of the therapeutic population of TILs are performed within a period of about 15 days to about 16 days.

In some embodiments, steps of the priming first expansion through the harvesting of the therapeutic population of TILs are performed within a period of about 14 days.

In some embodiments, steps of the priming first expansion through the harvesting of the therapeutic population of TILs are performed within a period of about 15 days.

In some embodiments, steps the priming first expansion through the harvesting of the therapeutic population of TILs are performed within a period of about 16 days.

In some embodiments, the method further comprises the step of cryopreserving the harvested therapeutic population of TILs using a cryopreservation process, wherein steps of the priming first expansion through the harvesting of therapeutic population of TILs and cryopreservation are performed in 16 days or less.

In some embodiments, therapeutic population of TILs harvested in the step of harvesting of therapeutic population of TILs comprises sufficient TILs for a therapeutically effective dosage of the TILs.

In some embodiments, the number of TILs sufficient for a therapeutically effective dosage is from about 2.3×10¹⁰ to about 13.7×10¹⁰.

In some embodiments, the third population of TILs in the step of the rapid second expansion provides for increased efficacy, increased interferon-gamma production, and/or increased polyclonality.

In some embodiments, the third population of TILs in the step of the rapid second expansion provides for at least a one-fold to five-fold or more interferon-gamma production as compared to TILs prepared by a process longer than 16 days.

In some embodiments, the effector T cells and/or central memory T cells obtained from the third population of TILs in the step of the rapid second expansion exhibit increased CD8 and CD28 expression relative to effector T cells and/or central memory T cells obtained from the second population of TILs in the step of the priming first expansion.

In some embodiments, therapeutic population of TILs from the step of the harvesting of therapeutic population of TILs are infused into a patient.

In some embodiments, the method further comprises the step of cryopreserving the infusion bag comprising the harvested TIL population using a cryopreservation process.

In some embodiments, the cryopreservation process is performed using a 1:1 ratio of harvested TIL population to cryopreservation media.

In some embodiments, the antigen-presenting cells are peripheral blood mononuclear cells (PBMCs).

In some embodiments, the PBMCs are irradiated and allogeneic.

In some embodiments, the antigen-presenting cells are artificial antigen-presenting cells.

In some embodiments, the harvesting step is performed using a membrane-based cell processing system.

In some embodiments, the harvesting step is performed using a LOVO cell processing system.

In some embodiments, the multiple fragments comprise about 60 fragments, and wherein each fragment has a volume of about 27 mm³.

In some embodiments, the multiple fragments comprise about 30 to about 60 fragments with a total volume of about 1300 mm³ to about 1500 mm³.

In some embodiments, the multiple fragments comprise about 50 fragments with a total volume of about 1350 mm³.

In some embodiments, the multiple fragments comprise about 50 fragments with a total mass of about 1 gram to about 1.5 grams.

In some embodiments, the cell culture medium is provided in a container selected from the group consisting of a G-container and a Xuri cellbag.

In some embodiments, the IL-2 concentration is about 10,000 IU/mL to about 5,000 IU/mL.

In some embodiments, the IL-2 concentration is about 6,000 IU/mL.

In some embodiments, the infusion bag in step (d) is a HypoThermosol-containing infusion bag.

In some embodiments, the cryopreservation media comprises dimethlysulfoxide (DMSO).

In some embodiments, the wherein the cryopreservation media comprises 7% to 10% DMSO.

In some embodiments, the first period and the second period in step (c) are each individually performed within a period of 5 days, 6 days, or 7 days.

In some embodiments, the first period is performed within a period of 5 days, 6 days, or 7 days.

In some embodiments, the second period is performed within a period of 10 or 11 days.

In some embodiments, the first period and the second period are each individually performed within a period of 7 days.

In some embodiments, all steps are performed within a period of about 14 days to about 22 days.

In some embodiments, all steps are performed within a period of about 14 days to about 21 days.

In some embodiments, all steps are performed within a period of about 14 days to about 20 days.

In some embodiments, all steps are performed within a period of about 14 days to about 19 days.

In some embodiments, all steps are performed within a period of about 14 days to about 18 days.

In some embodiments, all steps are performed within a period of about 14 days to about 17 days.

In some embodiments, all steps are performed within a period of about 14 days to about 16 days.

In some embodiments, all steps are performed within a period of about 15 days to about 16 days.

In some embodiments, all steps are performed within a period of about 14 days.

In some embodiments, all steps are performed within a period of about 15 days.

In some embodiments, all steps are performed within a period of about 16 days.

In some embodiments, all steps and cryopreservation are performed in 16 days or less.

In some embodiments, therapeutic population of TILs harvested comprises sufficient TILs for a therapeutically effective dosage of the TILs.

In some embodiments, the number of TILs sufficient for a therapeutically effective dosage is from about 2.3×10¹⁰ to about 13.7×10¹⁰.

In some embodiments, the container in the priming first expansion step is larger than the container in the rapid second expansion step.

In some embodiments, the third population of TILs provides for increased efficacy, increased interferon-gamma production, and/or increased polyclonality.

In some embodiments, the third population of TILs provides for at least a one-fold to five-fold or more interferon-gamma production as compared to TILs prepared by a process longer than 16 days.

In some embodiments, the effector T cells and/or central memory T cells obtained from the third population of TILs exhibit increased CD8 and CD28 expression relative to effector T cells and/or central memory T cells obtained from the second population of cells.

In some embodiments, the harvested TILs are infused into a patient.

In some embodiments, the present provides a method for treating a subject with cancer, the method comprising administering expanded tumor infiltrating lymphocytes (TILs) comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for about 1 to 7, 8, 9, 10, or 11 days to obtain the second population of TILs;
(d) performing a rapid second expansion by supplementing the cell culture medium of the second population of TILs with additional IL-2, OKT-3, and APCs, to produce a third population of TILs, wherein the number of APCs added to the rapid second expansion is at least twice the number of APCs added in step (b), wherein the rapid second expansion is performed for about 1 to 11 days to obtain the third population of TILs, wherein the third population of TILs is a therapeutic population of TILs, wherein the rapid second expansion is performed in a container comprising a second gas-permeable surface area;
(e) harvesting therapeutic population of TILs obtained from step (c);
(f) transferring the harvested TIL population from step (d) to an infusion bag; and
(g) administering a therapeutically effective dosage of the TILs from step (e) to the subject.

In some embodiments, the number of TILs sufficient for administering a therapeutically effective dosage in step (g) is from about 2.3×10¹⁰ to about 13.7×10¹⁰.

In some embodiments, the PD-1, CD39, CD38, CD103, CD101, LAG3, TIM3 and/or TIGIT positive TILs are PD-1high, CD39high, CD38lo, CD103high/lo, CD101lo, LAG3high, TIM3high and/or TIGIThigh TILs.

In some embodiments, the selection of step (b) comprises the steps of (i) exposing the first population of TILs to an excess of a monoclonal anti-PD-1 IgG4 antibody that binds to PD-1 through an N-terminal loop outside the IgV domain of PD-1, (ii) adding an excess of an anti-IgG4 antibody conjugated to a fluorophore, and (iii) performing a flow-based cell sort based on the fluorophore to obtain a PD-1 enriched TIL population.

In some embodiments, the monoclonal anti-PD-1 IgG4 antibody is nivolumab or variants, fragments, or conjugates thereof.

In some embodiments, the anti-IgG4 antibody is clone anti-human IgG4, Clone HP6023.

In some embodiments, the antigen presenting cells (APCs) are PBMCs.

In some embodiments, prior to administering a therapeutically effective dosage of TIL cells in step (g), a non-myeloablative lymphodepletion regimen has been administered to the subject.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the method further comprises the step of treating the patient with a high-dose IL-2 regimen starting on the day after administration of the TIL cells to the subject in step (g).

In some embodiments, the high-dose IL-2 regimen comprises 600,000 or 720,000 IU/kg administered as a 15-minute bolus intravenous infusion every eight hours until tolerance.

In some embodiments, the third population of TILs in step (c) provides for increased efficacy, increased interferon-gamma production, and/or increased polyclonality.

In some embodiments, the third population of TILs in step (d) provides for at least a one-fold to five-fold or more interferon-gamma production as compared to TILs prepared by a process longer than 16 days.

In some embodiments, the effector T cells and/or central memory T cells obtained from the third population of TILs exhibit increased CD8 and CD28 expression relative to effector T cells and/or central memory T cells obtained from the second population of TILs.

In some embodiments, the cancer is selected from the group consisting of melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma.

In some embodiments, the cancer is selected from the group consisting of melanoma, HNSCC, cervical cancers, NSCLC, glioblastoma (including GBM), and gastrointestinal cancer.

In some embodiments, the cancer is melanoma.

In some embodiments, the cancer is HNSCC.

In some embodiments, the cancer is a cervical cancer.

In some embodiments, the cancer is NSCLC.

In some embodiments, the cancer is glioblastoma (including GBM).

In some embodiments, the cancer is gastrointestinal cancer.

In some embodiments, the cancer is a hypermutated cancer.

In some embodiments, the cancer is a pediatric hypermutated cancer.

In some embodiments, the priming first expansion is performed in a first container and the rapid second expansion is performed in a second container, and wherein each of the first and second containers is a GREX-10.

In some embodiments, the priming first expansion is performed in a first closed container and the rapid second expansion is performed in a second closed container, and wherein each of the first and second closed containers comprises a GREX-100.

In some embodiments, the priming first expansion is performed in a first closed container and the rapid second expansion is performed in a second closed container, and wherein the each of the first and second closed containers comprises a GREX-500.

In some embodiments, the subject has been previously treated with an anti-PD-1 antibody.

In some embodiments, the subject has not been previously treated with an anti-PD-1 antibody.

In some embodiments, the priming first expansion step is performed on a first population of TILs selected or enriched for PD-1 positive TILs by contacting the first population of TILs with an anti-PD-1 antibody to form a first complex of the anti-PD-1 antibody and TIL cells in the first population of TILs, and then isolating the first complex to obtain the first population of TILs selected or enriched for PD-1 positive TILs.

In some embodiments, the anti-PD-1 antibody comprises an Fc region, wherein after the step of forming the first complexes and before the step of isolating the first complex the method further comprises the step of contacting the first complex with an anti-Fc antibody that binds to the Fc region of the anti-PD-1 antibody to form a second complex of the anti-Fc antibody and the first complex, and wherein the step of isolating the first complex is performed by isolating the second complex.

In some embodiments, the anti-PD-1 antibody is selected from the group consisting of EH12.2H7, PD1.3.1, SYM021, M1H4, A17188B, nivolumab (BMS-936558, Bristol-Myers Squibb; Opdivo®), pembrolizumab (lambrolizumab, MK03475 or MK-3475, Merck; Keytruda®), H12.1, PD1.3.1, NAT 105, humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), humanized anti-PD-1 IgG4 antibody PDR001 (Novartis), and RMP1-14 (rat IgG)—BioXcell cat #BP0146.

In some embodiments, the anti-PD-1 antibody is EH12.2H7.

In some embodiments, the anti-PD-1 antibody binds to a different epitope than nivolumab or pembrolizumab.

In some embodiments, the anti-PD-1 antibody binds to the same epitope as EH12.2H7 or nivolumab.

In some embodiments, the anti-PD-1 antibody is nivolumab.

In some embodiments, the subject has been previously treated with a first anti-PD1 antibody, wherein the priming first expansion step is performed on a first population of TILs selected or enriched for PD-1 positive TILs by contacting the first population of TILs with a second anti-PD-1 antibody to form a first complex of the second anti-PD-1 antibody and TIL cells in the first population of TILs, and then isolating the first complex to obtain the first population of TILs selected or enriched for PD-1 positive TILs, and wherein the second anti-PD-1 antibody is not blocked from binding to the first population of TILs by the first anti-PD-1 antibody insolubilized on the first population of TILs.

In some embodiments, the subject has been previously treated with a first anti-PD1 antibody, wherein the priming first expansion step is performed on a first population of TILs selected or enriched for PD-1 positive TILs by contacting the first population of TILs with a second anti-PD-1 antibody to form a first complex of the second anti-PD-1 antibody and TIL cells in the first population of TILs, and then isolating the first complex to obtain the first population of TILs selected or enriched for PD-1 positive TILs, and wherein the second anti-PD-1 antibody is blocked from binding to the first population of TILs by the first anti-PD-1 antibody insolubilized on the first population of TILs.

In some embodiments, the first anti-PD-1 antibody and the second anti-PD-1 antibody comprise an Fc region, wherein after the step of forming the first complex and before the step of isolating the first complex the method further comprises the step of contacting the first complex with an anti-Fc antibody that binds to the Fc region of the first anti-PD-1 antibody and the Fc region of the second anti-PD-1 antibody to form a second complex of the anti-Fc antibody and the first complex, and wherein the step of isolating the first complex is performed by isolating the second complex.

In some embodiments, the subject has been previously treated with a first anti-PD1 antibody, wherein the priming first expansion step is performed on a first population of TILs selected or enriched for PD-1 positive TILs by (i) contacting the first population of TILs with a second anti-PD-1 antibody to form a first complex of the second anti-PD-1 antibody and the first population of TILs, wherein the second anti-PD-1 antibody is blocked from binding to the PD-1 positive TILs by the first anti-PD-1 antibody insolubilized on the first population of TILs, wherein the first anti-PD-1 antibody and the second anti-PD-1 antibody comprise an Fc region, (ii) contacting the first complex with an anti-Fc antibody that binds to the Fc region of the second anti-PD-1 antibody to form a second complex of the anti-Fc antibody and the first complex and contacting the first anti-PD-1 antibody insolubilized on the first population of TILs with the anti-Fc antibody to form a third complex of the anti-Fc antibody and the first anti-PD-1 antibody insolubilized on the first population of TILs, and (iii) isolating the second and third complexes to obtain the first population of TILs selected or enriched for PD-1 positive TILs.

In some embodiments, the present invention provides a therapeutic population of tumor infiltrating lymphocytes (TILs) prepared from PD-1, LAG3, TIM3 and/or TIGIT positive cells selected from a digest of a tumor tissue sample obtained from a patient, wherein therapeutic population of TILs provides for increased efficacy and/or increased interferon-gamma production.

In some embodiments, therapeutic population of TILs provides for increased interferon-gamma production.

In some embodiments, therapeutic population of TILs provides for increased efficacy.

In some embodiments, therapeutic population of TILs is capable of at least one-fold more interferon-gamma production as compared to TILs prepared by a process longer than 16 days.

In some embodiments, therapeutic population of TILs is capable of at least one-fold more interferon-gamma production as compared to TILs prepared by a process longer than 16-22 days.

In some embodiments, the priming first expansion step is performed on a first population of TILs selected or enriched for PD-1, LAG3, TIM3 and/or TIGIT positive TILs with at least 11.27% to 74.4% PD-1 positive TILs.

In some embodiments, the priming first expansion step is performed on a first population of TILs selected or enriched for PD-1 positive TILs by the steps of:
(i) exposing the first population of TILs and a population of PBMC to an excess of a monoclonal anti-PD-1 IgG4 antibody that binds to PD-1 through an N-terminal loop outside the IgV domain of PD-1,
(ii) adding an excess of an anti-IgG4 antibody conjugated to a fluorophore, and
(iii) obtaining the first population of TILs selected or enriched for PD-1 positive TILs based on the intensity of the fluorophore of the PD-1 positive TILs in the first population of TILs compared to the intensity in the population of PBMCs as performed by fluorescence-activated cell sorting (FACS).

In some embodiments, the intensity of the fluorophore in both the first population of TILs and the population of PBMCs is used to set up FACS gates for establishing low, medium, and high levels of intensity that correspond to PD-1 negative TILs, PD-1 intermediate TILs, and PD-1 positive TILs, respectively.

In some embodiments, the FACS gates are set-up after step (a).

In some embodiments, the PD-1, LAG3, TIM3 and/or TIGIT positive TILs are PD-1high, LAG3high, TIM3high and/or TIGIThigh TILs.

In some embodiments, at least 80% of the first population of TILs selected or enriched for PD-1 positive TILs are PD-1 positive TILs, at least 80% of the first population of TILs selected or enriched for LAG3 positive TILs are LAG3 positive TILs, at least 80% of the first population of TILs selected or enriched for TIM3 positive TILs are TIM3 positive TILs, and/or at least 80% of the first population of TILs selected or enriched for TIGIT positive TILs are TIGIT positive TILs.

In some embodiments, the present provides a method for expanding tumor infiltrating lymphocytes (TILs) into a therapeutic population of TILs comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population, wherein at least a range of 10% to 80% of the first population of TILs are PD-1, CD39, CD38, CD103, CD101, LAG3, TIM3 and/or TIGIT positive TILs;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs;
(d) performing a rapid second expansion by supplementing the cell culture medium of the second population of TILs with additional IL-2, OKT-3, and APCs, to produce a third population of TILs, wherein the number of APCs added in the rapid second expansion is at least twice the number of APCs added in step (c), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(e) harvesting therapeutic population of TILs obtained from step (d); and
(f) transferring the harvested TIL population from step (e) to an infusion bag.

In some embodiments, selection of step (b) comprises the steps of:
(i) exposing the first population of TILs and a population of PBMC to an excess of a monoclonal anti-PD-1 IgG4 antibody that binds to PD-1 through an N-terminal loop outside the IgV domain of PD-1,
(ii) adding an excess of an anti-IgG4 antibody conjugated to a fluorophore,
(iii) obtaining the PD-1 enriched TIL population based on the intensity of the fluorophore of the PD-1 positive TILs in the first population of TILs compared to the intensity in the population of PBMCs as performed by fluorescence-activated cell sorting (FACS).

In some embodiments, the intensity of the fluorophore in both the first population and the population of PBMCs is used to set up FACS gates for establishing low, medium, and high levels of intensity that correspond to:
i) PD-1 negative TILs, PD-1 low TILs, PD-1 intermediate TILs, and PD-1 positive TILs, respectively;
ii) CD39 negative TILs, CD39 low TILs, CD39 intermediate TILs, and CD39 positive TILs, respectively;
iii) CD38 negative TILs, CD38 low TILs, CD38 intermediate TILs, and CD38 positive TILs, respectively;
iv) CD103 negative TILs, CD103 low TILs, CD103 intermediate TILs, and CD103 positive TILs, respectively;
v) CD101 negative TILs, CD101 low TILs, CD101 intermediate TILs, and CD101 positive TILs, respectively;
vi) LAG3 negative TILs, LAG3 low TILs, LAG3 intermediate TILs, and LAG3positive TILs, respectively;
vii) TIM3 negative TILs, TIM3 low TILs, TIM3 intermediate TILs, and TIM3 positive TILs, respectively; and/or
viii) TIGIT negative TILs, TIGIT low TILs, TIGIT intermediate TILs, and TIGIT positive TILs, respectively.

In some embodiments, the FACS gates are set-up after step (a).

In some embodiments, the PD-1, LAG3, TIM3 and/or TIGIT positive TILs are PD-1high, LAG3high, TIM3high and/or TIGIThigh TILs.

In some embodiments, at least 80% of the PD-1, LAG3, TIM3 and/or TIGIT enriched TIL population are PD-1, LAG3, TIM3 and/or TIGIT positive TILs.

In some embodiments, the third population of TILs comprises at least about 1×108 TILs in the container.

In some embodiments, the third population of TILs comprises at least about 1×109 TILs in the container.

In some embodiments, the number of PD-1, LAG3, TIM3 and/or TIGIT enriched TILs in the priming first expansion is from about 1×10⁴ to about 1×10⁶.

In some embodiments, the number of PD-1, LAG3, TIM3 and/or TIGIT enriched TILs in the priming first expansion is from about 5×10⁴ to about 1×10⁶.

In some embodiments, the number of PD-1, LAG3, TIM3 and/or TIGIT enriched TILs in the priming first expansion is from about 2×10⁵ to about 1×10⁶.

In some embodiments, the method further comprises the step of cyropreserving the first population of TILs from the tumor resected from the subject before performing step (a).

In some embodiments, the present provides a method for expanding tumor infiltrating lymphocytes (TILs) into a therapeutic population of TILs comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
(c) performing a priming first expansion by culturing the first population of TILs in a first TIL cell culture comprising a first cell culture medium, IL-2, and either:
   i) a first culture supernatant obtained from a first culture of antigen-presenting feeder cells (APCs), wherein the first culture supernatant comprises OKT-3, or
   ii) APCs and OKT-3, wherein the priming first expansion is performed by culturing the first TIL cell culture in a first container comprising a first gas-permeable surface area for a first period of about 1 to 7, 8, 9, 10, or 11 days to obtain a second population of TILs, and wherein the second population of TILs is greater in number than the first population of TILs;
(d) performing a rapid second expansion by transferring the first TIL cell culture into a second container comprising a second gas-permeable surface area supplemented with a second cell culture medium, IL-2, and either:
   i) a second culture supernatant obtained from a second culture of APCs, wherein the second culture supernatant comprises OKT-3, or
   ii) APCs and OKT-3;
to form a second TIL cell culture, wherein the rapid second expansion is performed by culturing the second TIL cell culture for a second period of about 1 to 11 days to obtain a third population of TILs, and wherein the third population of TILs is a therapeutic population of TILs; wherein the first TIL cell culture does not comprise both the first culture supernatant and APCs; wherein the second TIL cell culture does not comprise both the second culture supernatant and supplemental APCs;
(e) harvesting therapeutic population of TILs obtained from step (d); and
(f) transferring the harvested TIL population from step (e) to an infusion bag.

In some embodiments, the priming first expansion of step (c) the first TIL cell culture comprises the first culture supernatant, and wherein in the rapid second expansion of step (d) the first TIL cell culture is supplemented with OKT-3 and APCs to form the second TIL cell culture.

In some embodiments, in the priming first expansion of step (c) the first TIL cell culture comprises OKT-3 and APCs, and wherein in the rapid second expansion of step (d) the first TIL cell culture is supplemented with the second culture supernatant to form the second TIL cell culture.

In some embodiments, in the priming first expansion of step (c) the first TIL cell culture comprises the first culture supernatant, and wherein in the rapid second expansion of step (d) the first TIL cell culture is supplemented with the second culture supernatant to form the second TIL cell culture.

In some embodiments, obtaining the first culture supernatant for use in step (c) comprises:
1) providing an APC cell culture medium comprising IL-2 and OKT-3;
2) culturing at least about 5×10⁸ APCs in the APC cell culture medium from 1) for about 3-4 days to generate the first culture supernatant; and
3) collecting the first culture supernatant from the cell culture in 2).

In some embodiments, obtaining the second culture supernatant for use in step (d) comprises:
1) providing an APC cell culture medium comprising IL-2 and OKT-3;
2) culturing at least about 1×10⁷ APCs in the APC cell culture medium from 1) for about 3-4 days to generate the second culture supernatant; and
3) collecting the second culture supernatant from the cell culture in 2).

In some embodiments, the rapid second expansion of step (d) further comprises the step of:
i) supplementing the second TIL cell culture with additional IL-2 about 3 or 4 days after the initiation of the second period in step (d).

In some embodiments, the APCs are exogenous to the subject.

In some embodiments, the APCs are peripheral blood mononuclear cells (PBMCs).

In some embodiments, the rapid second expansion of step (d) further comprises the steps of:
i) on or about 3 or 4 days after the initiation of the second period, transferring the second TIL cell culture from the second container into a plurality of third containers to form a subculture of the second TIL cell culture in each of the plurality of third containers; and
ii) culturing the subculture of the second TIL cell culture in each of the plurality of third containers for the remainder of the second period.

In some embodiments, in step i) equal volumes of the second TIL cell culture are transferred into the plurality of third containers.

In some embodiments, each of the third containers is equal in size to the second container.

In some embodiments, each of the third containers is larger than the second container.

In some embodiments, the third containers are equal in size.

In some embodiments, the third containers are larger than the second container.

In some embodiments, the third containers are smaller than the second container.

In some embodiments, the second container is a G-Rex 100M flask.

In some embodiments, the second container is a G-Rex 100M flask and each of the plurality of third containers is a G-Rex 100M flask.

In some embodiments, the plurality of third containers is selected from the group consisting of: 2, 3, 4, 5, 6, 7, 8, 9, 10, 11, 12, 13, 14, 15, 16, 17, 18, 19 and 20 second containers.

In some embodiments, the plurality of second containers is 2 third containers.

In some embodiments, before step ii) the method further comprises supplementing each subculture of the second TIL cell culture with additional IL-2.

In some embodiments, before step ii) the method further comprises supplementing each subculture of the second TIL cell culture with a second cell culture medium and IL-2.

In some embodiments, the first cell culture medium and the second cell culture medium are the same.

In some embodiments, the first cell culture medium and the second cell culture medium are different.

In some embodiments, the first cell culture medium is DM1 and the second cell culture medium is DM2.

In some embodiments, the TILs are selected as PD-1 positive (PD-1+), LAG3 positive (LAG3+ positive), CD38 positive (CD38+), and CD101 positive (CD101+).

In some embodiments, the TILs are selected as PD-1high, LAG3high, CD38lo, and CD101lo.

In some embodiments, the TILs are selected as PD-1 positive (PD-1+), LAG3 positive (LAG3+ positive), and CD38 positive (CD38+).

In some embodiments, the TILs are selected as PD-1high, LAG3high, and CD38lo.

In some embodiments, the TILs are selected as PD-1 positive (PD-1+), LAG3 positive (LAG3+ positive), and CD101 positive (CD101+).

In some embodiments, the TILs are selected as PD-1high, LAG-3high, and CD101lo.

In some embodiments, the TILs are selected as PD-1 positive (PD-1+) and CD38 positive (CD38+),

In some embodiments, the TILs are selected as PD-1hi and CD38lo.

In some embodiments, the TILs are selected as PD-1 positive (PD-1+) and CD101 positive (CD101+).

In some embodiments, the TILs are selected as PD-1high and CD101lo.

In some embodiments, the selection comprises a selection method selected from the group consisting of flow cytometry (including for example FACS), antibody-based bead selection, and antibody-based magnetic bead selection.

In some embodiments, the selection method comprises flow cytometry (including for example FACS).

In some embodiments, the selection method comprises an antibody-based bead selection.

In some embodiments, the selection comprises an antibody-based magnetic bead selection.

In some embodiments, the selection comprises a two step selection, comprising:
i) a first selection step comprising a method that selects for PD-1+, LAG3+, TIM3+ and/or TIGIT+, and
ii) a second selection step comprising a method that selects for CD38+ and/or CD101+.

In some embodiments, the first selection step comprises a method that selects for PD-1high, LAG3high, TIM3high and/or TIGIThigh.

In some embodiments, the first selection step comprises a method that selects for PD-1high or LAG3high.

In some embodiments, the second selection step comprises a method that selects for CD3810 and/or CD101lo.

In some embodiments, the first selection step comprises flow cytometry (including for example FACS) and wherein the second selection step comprises flow cytometry (including for example FACS).

In some embodiments, the first selection step comprises an antibody-based bead selection and wherein the second selection step comprises flow cytometry (including for example FACS).

In some embodiments, the first selection step comprises an antibody-based magnetic bead selection and wherein the second selection step comprises flow cytometry (including for example FACS).

In some embodiments, the first selection step comprises an antibody-based bead selection or antibody-based magnetic bead selection and the second selection step comprises an antibody-based bead selection or antibody-based magnetic bead selection.

In some embodiments, the beads used in the antibody-based bead selection for PD-1+, LAG3+, TIM3+ and/or TIGIT+ TILs are anti-PD-1, anti-LAG3, anti-TIM3 and/or anti-TIGIT antibody conjugated beads, respectively.

In some embodiments, the beads used in the antibody-based bead selection for CD38+ or CD101+ TILs are anti-CD38 or anti-CD101 antibody conjugated beads, respectively.

In some embodiments, the beads used in the antibody-based magnetic bead selection for PD-1+, LAG3+, TIM3+ and/or TIGIT+ TILs are anti-PD-1, anti-LAG3, anti-TIM3 and/or anti-TIGIT antibody conjugated magnetic beads, respectively.

In some embodiments, the beads used in the antibody-based magnetic bead selection for CD38+ or CD101+ TILs are anti-CD38 or anti-CD101 antibody conjugated magnetic beads, respectively.

In some embodiments, the PD-1+, LAG3+, TIM3+ and/or TIGIT+ TILs bind to an anti-PD-1, anti-LAG3, anti-TIM3 and/or anti-TIGIT antibody conjugated beads, respectively, and the PD-1, LAG3, TIM3 and/or TIGIT negative TILs do not bind to an anti-PD-1, anti-LAG3, anti-TIM3 and/or anti-TIGIT antibody conjugated beads, respectively.

In some embodiments, the priming first expansion step is performed on a first population of TILs selected or enriched from a digest of a tumor sample obtained from a patient or subject.

In some embodiments, the digest is performed with a mixture of enzymes.

In some embodiments, the mixture of enzymes comprises a neutral protease, a collagenase, and a DNase.

### BRIEF DESCRIPTION OF THE DRAWINGS

FIG. 1A-1H: A) Shows a comparison between the 2A process (approximately 22-day process) and an embodiment of the PD-1 Gen 3 process for TIL manufacturing (approximately 14-days to 22-days process). B) Exemplary Process PD-1 Gen3 chart providing an overview of Steps A through F (approximately 14-days to 22-days process). C) Exemplary Process PD-1 Gen3 chart providing an overview of Steps A through F (approximately 14-days to 22-days process). D) Chart providing three exemplary Gen 3 processes with an overview of Steps A through F (approximately 14-days to 18-days process) for each of the three process variations. E) Chart providing three exemplary PD-1 Gen 3 processes with an overview of Steps A through F (approximately 14-days to 18-days process) for each of the three process variations. F) Chart providing exemplary PD-1 Gen 3 processes with an overview of Steps A through F (approximately 14-days to 18-days process). G) Chart providing exemplary PD-1 Gen 3 processes with an overview of Steps A through F (approximately 14-days to 18-days process). H) Chart providing exemplary PD-1 Gen 3 processes with an overview of Steps A through F (approximately 14-days to 18-days process).

FIG. 2: Provides an experimental flow chart for comparability between GEN 2 (process 2A) versus PD-1 GEN 3.

FIG. 3: Shows a comparison between various Gen 2 (2A process) and the Gen 3.1 process embodiment.

FIG. 4: Table describing various features of embodiments of the Gen 2, Gen 2.1 and Gen 3.0 process.

FIG. 5: Overview of the media conditions for an embodiment of the Gen 3 process, referred to as Gen 3.1.

FIG. 6: Schematic of an exemplary embodiment of the Gen 3 process (a 16-day process).

FIG. 7: Schematic diagram of PD-1 selection prior to expansion.

FIG. 8: Binding structure of nivolumab with PD-1. See, FIG. 5 from Tan, S. et al. (Tan, S. et al., Nature Communications, 8:14369 DOI: 10.1038/ncomms14369 (2017)).

FIG. 9: Binding structure of pembrolizumab with PD-1. See, FIG. 5 from Tan, S. et al. (Tan, S. et al., Nature Communications, 8:14369 DOI: 10.1038/ncomms14369 (2017)).

FIG. 10: A streamlined protocol was developed to expand PD1+ TIL to clinically relevant levels. The tumor is excised from the patient and transported to research laboratories. Upon arrival, the tumor is digested, and the single-cell suspension stained for CD3 and PD1. PD1+ TIL are sorted by FACS using an FX500 instrument (Sony). The PD1+ cell fraction is placed into a flask with an anti-human CD3 antibody (OKT3; 30 ng/ml) and irradiated allogeneic PBMCs (feeders) at 1:100 (TIL: feeder) ratio) and rapidly expanded for 22 days (REP).

FIG. 11: Identification of a tumor tissue digestion method.

FIG. 12: Schematic representation of exemplary embodiment for the tumor digestion and PD-1+ selection step, including PD-1 high selection.

FIG. 13: Schematic of an exemplary embodiment of a modified Gen 2 process developed for PD1 selected TIL.

FIG. 14: Schematic of an exemplary embodiments of a modified expansion processes developed for PD1 selected TIL.

FIG. 15: Schematic of an exemplary embodiments of a modified expansion processes developed for PD1 selected TIL.

FIG. 16: Schematic of a Full-Scale Processes embodiments for PD1 TIL culture.

FIG. 17: Small-Scale Process Embodiment: PD1-A is the condition that uses the Nivolumab staining procedure outlined in this protocol. PD1-B is the condition that uses the anti-PD1-PE (Clone #EH12.2H7) staining method. Bulk condition serves as a control.

FIG. 18: Overview of an embodiment of the PD-1+High Gen-2 Process.

FIG. 19: Exemplary Embodiments of Processes for PD-1+ TIL Culture (Research/PD-1+ Gen 2/Defined Media/Early REP).

FIG. 20: Provides the structures I-A and I-B, the cylinders refer to individual polypeptide binding domains. Structures I-A and I-B comprise three linearly-linked TNFRSF binding domains derived from e.g., 4-1BBL or an antibody that binds 4-1BB, which fold to form a trivalent protein, which is then linked to a second trivalent protein through IgG1-Fc (including CH3 and CH2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a VH and a VL chain connected by a linker that may comprise hydrophilic residues and Gly and Ser sequences for flexibility, as well as Glu and Lys for solubility.

FIG. 21: Identification of PD-1high TIL and expansion of PD-1-selected TIL in numerous cancer types.

FIG. 22: Ex vivo expanded PD-1-selected TIL demonstrated autologous tumor reactivity.

FIG. 23: Ex vivo expanded PD1+ TIL demonstrated effector activity in several in vitro assays. Data indicates that PD1+-selected TIL are antigen-specific and have greater effector function.

FIG. 24: Schematic if exemplary selection and expansion protocol.

FIG. 25: Data showing phenotypic properties of CD39+ positive cells.

FIG. 25: Data showing properties of CD39+ positive cells.

FIG. 26: Data showing results of phenotypic assessment in tumor digests of PD-1 selected TILs.

FIG. 27: Data showing results of phenotypic assessment in tumor digests of PD-1 selected TILs.

FIG. 28: PD1+ selected Gen-2 Process Overview.

FIG. 29: Phase-1 Experiment overview.

FIG. 30: Phase-2 Experiment overview of Full Scale PD1+ selected Gen-2 Process.

FIG. 31: Data showing cell population gating for Example 7.

FIG. 32: Data showing cell population gating for Example 7.

FIG. 33: Schematic of two exemplary PD-1 selection methods.

FIG. 34: Anti-PD-1 microbead conjugation and detection.

FIG. 35: Data showing >85% Purity was obtained when Magnetic Selection of PD-1+ TIL using anti-PD-1 (EH12.2H7). 2e6 REP TIL was added to 10 ul of cocktail (EH12.2H7)+5 ul of microbeads, incubated for 15 min at RT, incubated in magnet for 1 min at RT, Positively selection using magnet two times. Post sorted cells were stained with secondary mIgG1-PE, aCD3 FITC.

FIG. 36: Data showing >85% Purity was obtained when Magnetic Selection of PD-1+ TIL using anti-PD-1 (M1H4).

FIG. 37: Experimental Design. Comparison of two exemplary selection embodiments: Flow Sort versus Magnetic sort.

FIG. 38: Post sort TVC yields using Magnetic method (EH12.2H7) were higher than the flow sort method.

FIG. 39: PD-1 selected TIL growth characteristic, Identity, Function of magnetic selected TIL were comparable to flow sorted TIL.

FIG. 40: Differentiation, Activation and Exhaustion (CD4+) phenotypic markers were comparable.

FIG. 41: Differentiation, Activation and Exhaustion (CD8+) phenotypic markers were comparable.

FIG. 42: >99% of TCR Vbeta clones of Flow-sorted PD-1 selected TIL were present in Magnetic sorted (EH12.H7). Data shows the unique CDR3 counts and Shannon Diversity index of all the Test samples.

FIG. 43: >99% of TCR Vbeta clones of Flow-sorted PD-1 selected TIL were present in Magnetic sorted (EH12.H7). Data shows the overlapping uCDR3 samples between the test samples.

FIG. 44: Preselection with CD39. PD1^{high}CD39⁻ were mostly composed of CD4+ cells. PD1^{high}CD39⁺ had significantly reduced levels of CD69, compared to unselected TIL. PD-1^{high}CD39⁺ had significantly reduced IFNγ secretion, compared to both unselected and PD-1^{high}CD39⁻ TIL, when stimulated with anti-CD3/anti-CD28/anti-41BB. IFNγ secretion in response to autologous tumor was detected in the PD-1^{high}CD39⁺ TIL in 4 of 6 evaluable tumors (2 tumors were not assessed.)

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is the amino acid sequence of a recombinant human IL-4 protein.

SEQ ID NO:6 is the amino acid sequence of a recombinant human IL-7 protein.

SEQ ID NO:7 is the amino acid sequence of a recombinant human IL-15 protein.

SEQ ID NO:8 is the amino acid sequence of a recombinant human IL-21 protein.

SEQ ID NO:9 is the amino acid sequence of human 4-1BB.

SEQ ID NO:10 is the amino acid sequence of murine 4-1BB,

SEQ ID NO:11 is the heavy chain for the 4-1BB agonist monoclonal antibody utomilumab (PF-05082566).

SEQ ID NO:12 is the light chain for the 4-1BB agonist monoclonal antibody utomilumab (PF-05082566).

SEQ ID NO:13 is the heavy chain variable region (VH) for the 4-1BB agonist monoclonal antibody utomilumab (PF-05082566).

SEQ ID NO:14 is the light chain variable region (VL) for the 4-1BB agonist monoclonal antibody utomilumab (PF-05082566).

SEQ ID NO:15 is the heavy chain CDR1 for the 4-1BB agonist monoclonal antibody utomilumab (PF-05082566).

SEQ ID NO:16 is the heavy chain CDR2 for the 4-1BB agonist monoclonal antibody utomilumab (PF-05082566).

SEQ ID NO:17 is the heavy chain CDR3 for the 4-1BB agonist monoclonal antibody utomilumab (PF-05082566).

SEQ ID NO:18 is the light chain CDR1 for the 4-1BB agonist monoclonal antibody utomilumab (PF-05082566).

SEQ ID NO:19 is the light chain CDR2 for the 4-1BB agonist monoclonal antibody utomilumab (PF-05082566).

SEQ ID NO:20 is the light chain CDR3 for the 4-1BB agonist monoclonal antibody utomilumab (PF-05082566).

SEQ ID NO:21 is the heavy chain for the 4-1BB agonist monoclonal antibody urelumab (BMS-663513).

SEQ ID NO:22 is the light chain for the 4-1BB agonist monoclonal antibody urelumab (BMS-663513).

SEQ ID NO:23 is the heavy chain variable region (VH) for the 4-1BB agonist monoclonal antibody urelumab (BMS-663513).

SEQ ID NO:24 is the light chain variable region (VL) for the 4-1BB agonist monoclonal antibody urelumab (BMS-663513).

SEQ ID NO:25 is the heavy chain CDR1 for the 4-1BB agonist monoclonal antibody urelumab (BMS-663513).

SEQ ID NO:26 is the heavy chain CDR2 for the 4-1BB agonist monoclonal antibody urelumab (BMS-663513).

SEQ ID NO:27 is the heavy chain CDR3 for the 4-1BB agonist monoclonal antibody urelumab (BMS-663513).

SEQ ID NO:28 is the light chain CDR1 for the 4-1BB agonist monoclonal antibody urelumab (BMS-663513).

SEQ ID NO:29 is the light chain CDR2 for the 4-1BB agonist monoclonal antibody urelumab (BMS-663513).

SEQ ID NO:30 is the light chain CDR3 for the 4-1BB agonist monoclonal antibody urelumab (BMS-663513).

SEQ ID NO:31 is an Fc domain for a TNFRSF agonist fusion protein.

SEQ ID NO:32 is a linker for a TNFRSF agonist fusion protein.

SEQ ID NO:33 is a linker for a TNFRSF agonist fusion protein.

SEQ ID NO:34 is a linker for a TNFRSF agonist fusion protein.

SEQ ID NO:35 is a linker for a TNFRSF agonist fusion protein.

SEQ ID NO:36 is a linker for a TNFRSF agonist fusion protein.

SEQ ID NO:37 is a linker for a TNFRSF agonist fusion protein.

SEQ ID NO:38 is a linker for a TNFRSF agonist fusion protein.

SEQ ID NO:39 is a linker for a TNFRSF agonist fusion protein.

SEQ ID NO:40 is a linker for a TNFRSF agonist fusion protein.

SEQ ID NO:41 is a linker for a TNFRSF agonist fusion protein.

SEQ ID NO:42 is an Fc domain for a TNFRSF agonist fusion protein.

SEQ ID NO:43 is a linker for a TNFRSF agonist fusion protein.

SEQ ID NO:44 is a linker for a TNFRSF agonist fusion protein.

SEQ ID NO:45 is a linker for a TNFRSF agonist fusion protein.

SEQ ID NO:46 is a 4-1BB ligand (4-1BBL) amino acid sequence.

SEQ ID NO:47 is a soluble portion of 4-1BBL polypeptide.

SEQ ID NO:48 is a heavy chain variable region (VH) for the 4-1BB agonist antibody 4B4-1-1 version 1.

SEQ ID NO:49 is a light chain variable region (VL) for the 4-1BB agonist antibody 4B4-1-1 version 1.

SEQ ID NO:50 is a heavy chain variable region (VH) for the 4-1BB agonist antibody 4B4-1-1 version 2.

SEQ ID NO:51 is a light chain variable region (VL) for the 4-1BB agonist antibody 4B4-1-1 version 2.

SEQ ID NO:52 is a heavy chain variable region (VH) for the 4-1BB agonist antibody

H39E3-2.

SEQ ID NO:53 is a light chain variable region (VL) for the 4-1BB agonist antibody

H39E3-2.

SEQ ID NO:54 is the amino acid sequence of human OX40.

SEQ ID NO:55 is the amino acid sequence of murine OX40.

SEQ ID NO:56 is the heavy chain for the OX40 agonist monoclonal antibody tavolixizumab (MEDI-0562).

SEQ ID NO:57 is the light chain for the OX40 agonist monoclonal antibody tavolixizumab (MEDI-0562).

SEQ ID NO:58 is the heavy chain variable region (VH) for the OX40 agonist monoclonal antibody tavolixizumab (MEDI-0562).

SEQ ID NO:59 is the light chain variable region (VL) for the OX40 agonist monoclonal antibody tavolixizumab (MEDI-0562).

SEQ ID NO:60 is the heavy chain CDR1 for the OX40 agonist monoclonal antibody tavolixizumab (MEDI-0562).

SEQ ID NO:61 is the heavy chain CDR2 for the OX40 agonist monoclonal antibody tavolixizumab (MEDI-0562).

SEQ ID NO:62 is the heavy chain CDR3 for the OX40 agonist monoclonal antibody tavolixizumab (MEDI-0562).

SEQ ID NO:63 is the light chain CDR1 for the OX40 agonist monoclonal antibody tavolixizumab (MEDI-0562).

SEQ ID NO:64 is the light chain CDR2 for the OX40 agonist monoclonal antibody tavolixizumab (MEDI-0562).

SEQ ID NO:65 is the light chain CDR3 for the OX40 agonist monoclonal antibody tavolixizumab (MEDI-0562).

SEQ ID NO:66 is the heavy chain for the OX40 agonist monoclonal antibody 11D4.

SEQ ID NO:67 is the light chain for the OX40 agonist monoclonal antibody 11D4.

SEQ ID NO:68 is the heavy chain variable region (VH) for the OX40 agonist monoclonal antibody 11D4.

SEQ ID NO:69 is the light chain variable region (VL) for the OX40 agonist monoclonal antibody 11D4.

SEQ ID NO:70 is the heavy chain CDR1 for the OX40 agonist monoclonal antibody 11D4.

SEQ ID NO:71 is the heavy chain CDR2 for the OX40 agonist monoclonal antibody 11D4.

SEQ ID NO:72 is the heavy chain CDR3 for the OX40 agonist monoclonal antibody 11D4.

SEQ ID NO:73 is the light chain CDR1 for the OX40 agonist monoclonal antibody 11D4.

SEQ ID NO:74 is the light chain CDR2 for the OX40 agonist monoclonal antibody 11D4.

SEQ ID NO:75 is the light chain CDR3 for the OX40 agonist monoclonal antibody 11D4.

SEQ ID NO:76 is the heavy chain for the OX40 agonist monoclonal antibody 18D8.

SEQ ID NO:77 is the light chain for the OX40 agonist monoclonal antibody 18D8.

SEQ ID NO:78 is the heavy chain variable region (VH) for the OX40 agonist monoclonal antibody 18D8.

SEQ ID NO:79 is the light chain variable region (VL) for the OX40 agonist monoclonal antibody 18D8.

SEQ ID NO:80 is the heavy chain CDR1 for the OX40 agonist monoclonal antibody 18D8.

SEQ ID NO:81 is the heavy chain CDR2 for the OX40 agonist monoclonal antibody 18D8.

SEQ ID NO:82 is the heavy chain CDR3 for the OX40 agonist monoclonal antibody 18D8.

SEQ ID NO:83 is the light chain CDR1 for the OX40 agonist monoclonal antibody 18D8.

SEQ ID NO:84 is the light chain CDR2 for the OX40 agonist monoclonal antibody 18D8.

SEQ ID NO:85 is the light chain CDR3 for the OX40 agonist monoclonal antibody 18D8.

SEQ ID NO:86 is the heavy chain variable region (VH) for the OX40 agonist monoclonal antibody Hu119-122.

SEQ ID NO:87 is the light chain variable region (VL) for the OX40 agonist monoclonal antibody Hu119-122.

SEQ ID NO:88 is the heavy chain CDR1 for the OX40 agonist monoclonal antibody

Hu119-122.

SEQ ID NO:89 is the heavy chain CDR2 for the OX40 agonist monoclonal antibody

Hu119-122.

SEQ ID NO:90 is the heavy chain CDR3 for the OX40 agonist monoclonal antibody

Hu119-122.

SEQ ID NO:91 is the light chain CDR1 for the OX40 agonist monoclonal antibody Hu119-122.

SEQ ID NO:92 is the light chain CDR2 for the OX40 agonist monoclonal antibody Hu119-122.

SEQ ID NO:93 is the light chain CDR3 for the OX40 agonist monoclonal antibody Hu119-122.

SEQ ID NO:94 is the heavy chain variable region (VH) for the OX40 agonist monoclonal antibody Hu106-222.

SEQ ID NO:95 is the light chain variable region (VL) for the OX40 agonist monoclonal antibody Hu106-222.

SEQ ID NO:96 is the heavy chain CDR1 for the OX40 agonist monoclonal antibody

Hu106-222.

SEQ ID NO:97 is the heavy chain CDR2 for the OX40 agonist monoclonal antibody

Hu106-222.

SEQ ID NO:98 is the heavy chain CDR3 for the OX40 agonist monoclonal antibody

Hu106-222.

SEQ ID NO:99 is the light chain CDR1 for the OX40 agonist monoclonal antibody Hu106-222.

SEQ ID NO:100 is the light chain CDR2 for the OX40 agonist monoclonal antibody

Hu106-222.

SEQ ID NO:101 is the light chain CDR3 for the OX40 agonist monoclonal antibody

Hu106-222.

SEQ ID NO:102 is an OX40 ligand (OX40L) amino acid sequence.

SEQ ID NO:103 is a soluble portion of OX40L polypeptide.

SEQ ID NO:104 is an alternative soluble portion of OX40L polypeptide.

SEQ ID NO:105 is the heavy chain variable region (VH) for the OX40 agonist monoclonal antibody 008.

SEQ ID NO:106 is the light chain variable region (VL) for the OX40 agonist monoclonal antibody 008.

SEQ ID NO:107 is the heavy chain variable region (VH) for the OX40 agonist monoclonal antibody 011.

SEQ ID NO:108 is the light chain variable region (VL) for the OX40 agonist monoclonal antibody 011.

SEQ ID NO:109 is the heavy chain variable region (VH) for the OX40 agonist monoclonal antibody 021.

SEQ ID NO:110 is the light chain variable region (VL) for the OX40 agonist monoclonal antibody 021.

SEQ ID NO:111 is the heavy chain variable region (VH) for the OX40 agonist monoclonal antibody 023.

SEQ ID NO:112 is the light chain variable region (VL) for the OX40 agonist monoclonal antibody 023.

SEQ ID NO:113 is the heavy chain variable region (VH) for an OX40 agonist monoclonal antibody.

SEQ ID NO:114 is the light chain variable region (VL) for an OX40 agonist monoclonal antibody.

SEQ ID NO:115 is the heavy chain variable region (VH) for an OX40 agonist monoclonal antibody.

SEQ ID NO:116 is the light chain variable region (VL) for an OX40 agonist monoclonal antibody.

SEQ ID NO:117 is the heavy chain variable region (VH) for a humanized OX40 agonist monoclonal antibody.

SEQ ID NO:118 is the heavy chain variable region (VH) for a humanized OX40 agonist monoclonal antibody.

SEQ ID NO:119 is the light chain variable region (VL) for a humanized OX40 agonist monoclonal antibody.

SEQ ID NO:120 is the light chain variable region (VL) for a humanized OX40 agonist monoclonal antibody.

SEQ ID NO:121 is the heavy chain variable region (VH) for a humanized OX40 agonist monoclonal antibody.

SEQ ID NO:122 is the heavy chain variable region (VH) for a humanized OX40 agonist monoclonal antibody.

SEQ ID NO:123 is the light chain variable region (VL) for a humanized OX40 agonist monoclonal antibody.

SEQ ID NO:124 is the light chain variable region (VL) for a humanized OX40 agonist monoclonal antibody.

SEQ ID NO:125 is the heavy chain variable region (VH) for an OX40 agonist monoclonal antibody.

SEQ ID NO:126 is the light chain variable region (VL) for an OX40 agonist monoclonal antibody.

SEQ ID NO:127-462 are currently not assigned.

SEQ ID NO:463 is the heavy chain amino acid sequence of the PD-1 inhibitor nivolumab.

SEQ ID NO:464 is the light chain amino acid sequence of the PD-1 inhibitor nivolumab.

SEQ ID NO:465 is the heavy chain variable region (VH) amino acid sequence of the PD-1 inhibitor nivolumab.

SEQ ID NO:466 is the light chain variable region (VL) amino acid sequence of the PD-1 inhibitor nivolumab.

SEQ ID NO:467 is the heavy chain CDR1 amino acid sequence of the PD-1 inhibitor nivolumab.

SEQ ID NO:468 is the heavy chain CDR2 amino acid sequence of the PD-1 inhibitor nivolumab.

SEQ ID NO:469 is the heavy chain CDR3 amino acid sequence of the PD-1 inhibitor nivolumab.

SEQ ID NO:470 is the light chain CDR1 amino acid sequence of the PD-1 inhibitor nivolumab.

SEQ ID NO:471 is the light chain CDR2 amino acid sequence of the PD-1 inhibitor nivolumab.

SEQ ID NO:472 is the light chain CDR3 amino acid sequence of the PD-1 inhibitor nivolumab.

SEQ ID NO:473 is the heavy chain amino acid sequence of the PD-1 inhibitor pembrolizumab.

SEQ ID NO:474 is the light chain amino acid sequence of the PD-1 inhibitor pembrolizumab.

SEQ ID NO:475 is the heavy chain variable region (VH) amino acid sequence of the PD-1 inhibitor pembrolizumab.

SEQ ID NO:476 is the light chain variable region (VL) amino acid sequence of the PD-1 inhibitor pembrolizumab.

SEQ ID NO:477 is the heavy chain CDR1 amino acid sequence of the PD-1 inhibitor pembrolizumab.

SEQ ID NO:478 is the heavy chain CDR2 amino acid sequence of the PD-1 inhibitor pembrolizumab.

SEQ ID NO:479 is the heavy chain CDR3 amino acid sequence of the PD-1 inhibitor pembrolizumab.

SEQ ID NO:480 is the light chain CDR1 amino acid sequence of the PD-1 inhibitor pembrolizumab.

SEQ ID NO:481 is the light chain CDR2 amino acid sequence of the PD-1 inhibitor pembrolizumab.

SEQ ID NO:482 is the light chain CDR3 amino acid sequence of the PD-1 inhibitor pembrolizumab.

SEQ ID NO:483 is the heavy chain amino acid sequence of the PD-L1 inhibitor durvalumab.

SEQ ID NO:484 is the light chain amino acid sequence of the PD-L1 inhibitor durvalumab.

SEQ ID NO:485 is the heavy chain variable region (VH) amino acid sequence of the PD-L1 inhibitor durvalumab.

SEQ ID NO:486 is the light chain variable region (VL) amino acid sequence of the PD-L1 inhibitor durvalumab.

SEQ ID NO:487 is the heavy chain CDR1 amino acid sequence of the PD-L1 inhibitor durvalumab.

SEQ ID NO:488 is the heavy chain CDR2 amino acid sequence of the PD-L1 inhibitor durvalumab.

SEQ ID NO:489 is the heavy chain CDR3 amino acid sequence of the PD-L1 inhibitor durvalumab.

SEQ ID NO:490 is the light chain CDR1 amino acid sequence of the PD-L1 inhibitor durvalumab.

SEQ ID NO:491 is the light chain CDR2 amino acid sequence of the PD-L1 inhibitor durvalumab.

SEQ ID NO:492 is the light chain CDR3 amino acid sequence of the PD-L1 inhibitor durvalumab.

SEQ ID NO:493 is the heavy chain amino acid sequence of the PD-L1 inhibitor avelumab.

SEQ ID NO:494 is the light chain amino acid sequence of the PD-L1 inhibitor avelumab.

SEQ ID NO:495 is the heavy chain variable region (VH) amino acid sequence of the PD-L1 inhibitor avelumab.

SEQ ID NO:496 is the light chain variable region (VL) amino acid sequence of the PD-L1 inhibitor avelumab.

SEQ ID NO:497 is the heavy chain CDR1 amino acid sequence of the PD-L1 inhibitor avelumab.

SEQ ID NO:498 is the heavy chain CDR2 amino acid sequence of the PD-L1 inhibitor avelumab.

SEQ ID NO:499 is the heavy chain CDR3 amino acid sequence of the PD-L1 inhibitor avelumab.

SEQ ID NO:500 is the light chain CDR1 amino acid sequence of the PD-L1 inhibitor avelumab.

SEQ ID NO:501 is the light chain CDR2 amino acid sequence of the PD-L1 inhibitor avelumab.

SEQ ID NO:502 is the light chain CDR3 amino acid sequence of the PD-L1 inhibitor avelumab.

SEQ ID NO:503 is the heavy chain amino acid sequence of the PD-L1 inhibitor atezolizumab.

SEQ ID NO:504 is the light chain amino acid sequence of the PD-L1 inhibitor atezolizumab.

SEQ ID NO:505 is the heavy chain variable region (VH) amino acid sequence of the PD-L1 inhibitor atezolizumab.

SEQ ID NO:506 is the light chain variable region (VL) amino acid sequence of the PD-L1 inhibitor atezolizumab.

SEQ ID NO:507 is the heavy chain CDR1 amino acid sequence of the PD-L1 inhibitor atezolizumab.

SEQ ID NO:508 is the heavy chain CDR2 amino acid sequence of the PD-L1 inhibitor atezolizumab.

SEQ ID NO:509 is the heavy chain CDR3 amino acid sequence of the PD-L1 inhibitor atezolizumab.

SEQ ID NO:510 is the light chain CDR1 amino acid sequence of the PD-L1 inhibitor atezolizumab.

SEQ ID NO:511 is the light chain CDR2 amino acid sequence of the PD-L1 inhibitor atezolizumab.

SEQ ID NO:512 is the light chain CDR3 amino acid sequence of the PD-L1 inhibitor atezolizumab.

SEQ ID NO:513 is the heavy chain amino acid sequence of the CTLA-4 inhibitor ipilimumab.

SEQ ID NO:514 is the light chain amino acid sequence of the CTLA-4 inhibitor ipilimumab.

SEQ ID NO:515 is the heavy chain variable region (VH) amino acid sequence of the CTLA-4 inhibitor ipilimumab.

SEQ ID NO:516 is the light chain variable region (VL) amino acid sequence of the CTLA-4 inhibitor ipilimumab.

SEQ ID NO:517 is the heavy chain CDR1 amino acid sequence of the CTLA-4 inhibitor ipilimumab.

SEQ ID NO:518 is the heavy chain CDR2 amino acid sequence of the CTLA-4 inhibitor ipilimumab.

SEQ ID NO:519 is the heavy chain CDR3 amino acid sequence of the CTLA-4 inhibitor ipilimumab.

SEQ ID NO:520 is the light chain CDR1 amino acid sequence of the CTLA-4 inhibitor ipilimumab.

SEQ ID NO:521 is the light chain CDR2 amino acid sequence of the CTLA-4 inhibitor ipilimumab.

SEQ ID NO:522 is the light chain CDR3 amino acid sequence of the CTLA-4 inhibitor ipilimumab.

SEQ ID NO:523 is the heavy chain amino acid sequence of the CTLA-4 inhibitor tremelimumab.

SEQ ID NO:524 is the light chain amino acid sequence of the CTLA-4 inhibitor tremelimumab.

SEQ ID NO:525 is the heavy chain variable region (VH) amino acid sequence of the CTLA-4 inhibitor tremelimumab.

SEQ ID NO:526 is the light chain variable region (VL) amino acid sequence of the CTLA-4 inhibitor tremelimumab.

SEQ ID NO:527 is the heavy chain CDR1 amino acid sequence of the CTLA-4 inhibitor tremelimumab.

SEQ ID NO:528 is the heavy chain CDR2 amino acid sequence of the CTLA-4 inhibitor tremelimumab.

SEQ ID NO:529 is the heavy chain CDR3 amino acid sequence of the CTLA-4 inhibitor tremelimumab.

SEQ ID NO:530 is the light chain CDR1 amino acid sequence of the CTLA-4 inhibitor tremelimumab.

SEQ ID NO:531 is the light chain CDR2 amino acid sequence of the CTLA-4 inhibitor tremelimumab.

SEQ ID NO:532 is the light chain CDR3 amino acid sequence of the CTLA-4 inhibitor tremelimumab.

SEQ ID NO:533 is the heavy chain amino acid sequence of the CTLA-4 inhibitor zalifrelimab.

SEQ ID NO:534 is the light chain amino acid sequence of the CTLA-4 inhibitor zalifrelimab.

SEQ ID NO:535 is the heavy chain variable region (VH) amino acid sequence of the CTLA-4 inhibitor zalifrelimab.

SEQ ID NO:536 is the light chain variable region (VL) amino acid sequence of the CTLA-4 inhibitor zalifrelimab.

SEQ ID NO:537 is the heavy chain CDR1 amino acid sequence of the CTLA-4 inhibitor zalifrelimab.

SEQ ID NO:538 is the heavy chain CDR2 amino acid sequence of the CTLA-4 inhibitor zalifrelimab.

SEQ ID NO:539 is the heavy chain CDR3 amino acid sequence of the CTLA-4 inhibitor zalifrelimab.

SEQ ID NO:540 is the light chain CDR1 amino acid sequence of the CTLA-4 inhibitor zalifrelimab.

SEQ ID NO:541 is the light chain CDR2 amino acid sequence of the CTLA-4 inhibitor zalifrelimab.

SEQ ID NO:542 is the light chain CDR3 amino acid sequence of the CTLA-4 inhibitor zalifrelimab.

SEQ ID NO:543 is the IL-2 sequence.

SEQ ID NO:544 is an IL-2 mutein sequence.

SEQ ID NO:545 is an IL-2 mutein sequence.

SEQ ID NO:546 is the HCDR1_IL-2 for IgG.IL2R67A.H1.

SEQ ID NO:547 is the HCDR2 for IgG.IL2R67A.H1.

SEQ ID NO:548 is the HCDR3 for IgG.IL2R67A.H1.

SEQ ID NO:549 is the HCDR1_IL-2 kabat for IgG.IL2R67A.H1.

SEQ ID NO:550 is the HCDR2 kabat for IgG.IL2R67A.H1.

SEQ ID NO:551 is the HCDR3 kabat for IgG.IL2R67A.H1.

SEQ ID NO:552 is the HCDR1_IL-2 clothia for IgG.IL2R67A.H1.

SEQ ID NO:553 is the HCDR2 clothia for IgG.IL2R67A.H1.

SEQ ID NO:554 is the HCDR3 clothia for IgG.IL2R67A.H1.

SEQ ID NO:555 is the HCDR1_IL-2 IMGT for IgG.IL2R67A.H1.

SEQ ID NO:556 is the HCDR2 IMGT for IgG.IL2R67A.H1.

SEQ ID NO:557 is the HCDR3 IMGT for IgG.IL2R67A.H1.

SEQ ID NO:558 is the VH chain for IgG.IL2R67A.H1.

SEQ ID NO:559 is the heavy chain for IgG.IL2R67A.H1.

SEQ ID NO:560 is the LCDR1 kabat for IgG.IL2R67A.H1.

SEQ ID NO:561 is the LCDR2 kabat for IgG.IL2R67A.H1.

SEQ ID NO:562 is the LCDR3 kabat for IgG.IL2R67A.H1.

SEQ ID NO:563 is the LCDR1 chothia for IgG.IL2R67A.H1.

SEQ ID NO:564 is the LCDR2 chothia for IgG.IL2R67A.H1.

SEQ ID NO:565 is the LCDR3 chothia for IgG.IL2R67A.H1.

SEQ ID NO:566 is the VL chain.

SEQ ID NO:567 is the light chain.

SEQ ID NO:568 is the light chain.

SEQ ID NO:569 is the light chain.

I. Definitions

Unless defined otherwise, all technical and scientific terms used herein have the same meaning as is commonly understood by one of skill in the art to which this invention belongs. All patents and publications referred to herein are incorporated by reference in their entireties.

The term “in vivo” refers to an event that takes place in a subject's body.

The term “in vitro” refers to an event that takes places outside of a subjects body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are outlined below.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly obtained” or “freshly isolated”), and “secondary TILs” are any TIL cell populations that have been expanded or proliferated as discussed herein, including, but not limited to bulk TILs and expanded TILs (“REP TILs” or “post-REP TILs”). TIL cell populations can include genetically modified TILs.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion.

By “cryopreserved TILs” herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally and alternatively, TILs can be functionally defined by their ability to infiltrate solid tumors upon reintroduction into a patient.

The term “cryopreservation media” or “cryopreservation medium” refers to any medium that can be used for cryopreservation of cells. Such media can include media comprising 7% to 10% DMSO. Exemplary media include CryoStor CS10, Hyperthermasol, as well as combinations thereof. The term “CS10” refers to a cryopreservation medium which is obtained from Stemcell Technologies or from Biolife Solutions. The CS10 medium may be referred to by the trade name “CryoStor® CS10”. The CS10 medium is a serum-free, animal component-free medium which comprises DMSO.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7) (CCR7^{lo} and are heterogeneous or low for CD62L expression)(CD62L^{lo}. The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perforin.

The term “closed system” refers to a system that is closed to the outside environment. Any closed system appropriate for cell culture methods can be employed with the methods of the present invention. Closed systems include, for example, but are not limited to closed G-containers. Once a tumor segment is added to the closed system, the system is no opened to the outside environment until the TILs are ready to be administered to the patient.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. When used as antigen-presenting cells (PBMCs are a type of antigen-presenting cell), the peripheral blood mononuclear cells are preferably irradiated allogeneic peripheral blood mononuclear cells.

The terms “peripheral blood lymphocytes” and “PBLs” refer to T cells expanded from peripheral blood. In some embodiments, PBLs are separated from whole blood or apheresis product from a donor. In some embodiments, PBLs are separated from whole blood or apheresis product from a donor by positive or negative selection of a T cell phenotype, such as the T cell phenotype of CD3+ CD45+.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3E. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, Calif., USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab (exemplary OKT-3 antibody).

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| Muromonab heavy | NQKFKDKATL TTDKSSSTAY MQLSSLTSED SAVYYCARYY DDHYCLDYWG QGTTLTVSSA | 120 |
| chain | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| Muromonab light | FRGSGSGTSY SLTISGMEAE DAATYYCQQW SSNPFTFGSG TKLEINRADT APTVSIFPPS | 120 |
| chain | SEQLTSGGAS VVCFLNNFYP KDINVKWKID GSERQNGVLN SWIDQDSKDS TYSMSSTLTL | 130 |
| | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug bempegaldesleukin (NKTR-214, pegylated human recombinant IL-2 as in SEQ ID NO:4 in which an average of 6 lysine residues are N⁶ substituted with [(2,7-bis{[methylpoly(oxyethylene)]carbamoyl}-9H-fluoren-9-yl)methoxy]carbonyl), which is available from Nektar Therapeutics, South San Francisco, CA, USA, or which may be prepared by methods known in the art, such as the methods described in Example 19 of International Patent Application Publication No. WO 2018/132496 A1 or the method described in Example 1 of U.S. Patent Application Publication No. US 2019/0275133 A1, the disclosures of which are incorporated by reference herein. Bempegaldesleukin (NKTR-214) and other pegylated IL-2 molecules suitable for use in the invention are described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

In some embodiments, an IL-2 form suitable for use in the present invention is THOR-707, available from Synthorx, Inc. The preparation and properties of THOR-707 and additional alternative forms of IL-2 suitable for use in the invention are described in U.S. Patent Application Publication Nos. US 2020/0181220 A1 and US 2020/0330601 A1, the disclosures of which are incorporated by reference herein. In some embodiments, and IL-2 form suitable for use in the invention is an interleukin 2 (IL-2) conjugate comprising: an isolated and purified IL-2 polypeptide; and a conjugating moiety that binds to the isolated and purified IL-2 polypeptide at an amino acid position selected from K35, T37, R38, T41, F42, K43, F44, Y45, E61, E62, E68, K64, P65, V69, L72, and Y107, wherein the numbering of the amino acid residues corresponds to SEQ ID NO:5. In some embodiments, the amino acid position is selected from T37, R38, T41, F42, F44, Y45, E61, E62, E68, K64, P65, V69, L72, and Y107. In some embodiments, the amino acid position is selected from T37, R38, T41, F42, F44, Y45, E61, E62, E68, P65, V69, L72, and Y107. In some embodiments, the amino acid position is selected from T37, T41, F42, F44, Y45, P65, V69, L72, and Y107. In some embodiments, the amino acid position is selected from R38 and K64. In some embodiments, the amino acid position is selected from E61, E62, and E68. In some embodiments, the amino acid position is at E62. In some embodiments, the amino acid residue selected from K35, T37, R38, T41, F42, K43, F44, Y45, E61, E62, E68, K64, P65, V69, L72, and Y107 is further mutated to lysine, cysteine, or histidine. In some embodiments, the amino acid residue is mutated to cysteine. In some embodiments, the amino acid residue is mutated to lysine. In some embodiments, the amino acid residue selected from K35, T37, R38, T41, F42, K43, F44, Y45, E61, E62, E68, K64, P65, V69, L72, and Y107 is further mutated to an unnatural amino acid. In some embodiments, the unnatural amino acid comprises N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine. In some embodiments, the IL-2 conjugate has a decreased affinity to IL-2 receptor α (IL-2Ra) subunit relative to a wild-type IL-2 polypeptide. In some embodiments, the decreased affinity is about 10%, 20%, 30%, 40%, 50%, 60%, 70%, 80%, 90%, 95%, 99%, or greater than 99% decrease in binding affinity to IL-2Ra relative to a wild-type IL-2 polypeptide. In some embodiments, the decreased affinity is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 500-fold, 1000-fold, or more relative to a wild-type IL-2 polypeptide. In some embodiments, the conjugating moiety impairs or blocks the binding of IL-2 with IL-2Ra. In some embodiments, the conjugating moiety comprises a water-soluble polymer. In some embodiments, the additional conjugating moiety comprises a water-soluble polymer. In some embodiments, each of the water-soluble polymers independently comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), or a combination thereof. In some embodiments, each of the water-soluble polymers independently comprises PEG. In some embodiments, the PEG is a linear PEG or a branched PEG. In some embodiments, each of the water-soluble polymers independently comprises a polysaccharide. In some embodiments, the polysaccharide comprises dextran, polysialic acid (PSA), hyaluronic acid (HA), amylose, heparin, heparan sulfate (HS), dextrin, or hydroxyethyl-starch (HES). In some embodiments, each of the water-soluble polymers independently comprises a glycan. In some embodiments, each of the water-soluble polymers independently comprises polyamine. In some embodiments, the conjugating moiety comprises a protein. In some embodiments, the additional conjugating moiety comprises a protein. In some embodiments, each of the proteins independently comprises an albumin, a transferrin, or a transthyretin. In some embodiments, each of the proteins independently comprises an Fc portion. In some embodiments, each of the proteins independently comprises an Fc portion of IgG. In some embodiments, the conjugating moiety comprises a polypeptide. In some embodiments, the additional conjugating moiety comprises a polypeptide. In some embodiments, each of the polypeptides independently comprises a XTEN peptide, a glycine-rich homoamino acid polymer (HAP), a PAS polypeptide, an elastin-like polypeptide (ELP), a CTP peptide, or a gelatin-like protein (GLK) polymer. In some embodiments, the isolated and purified IL-2 polypeptide is modified by glutamylation. In some embodiments, the conjugating moiety is directly bound to the isolated and purified IL-2 polypeptide. In some embodiments, the conjugating moiety is indirectly bound to the isolated and purified IL-2 polypeptide through a linker. In some embodiments, the linker comprises a homobifunctional linker. In some embodiments, the homobifunctional linker comprises Lomant's reagent dithiobis (succinimidylpropionate) DSP, 3′3′-dithiobis(sulfosuccinimidyl proprionate) (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-(3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide). In some embodiments, the linker comprises a heterobifunctional linker. In some embodiments, the heterobifunctional linker comprises N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy) sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (slAXX), succinimidyl 4-(((iodoacetyl)amino)methyl)cyclohexane-1-carboxylate (sIAC), succinimidyl 6-(((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenyl amino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4 (4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), 1-(p-azidosalicylamido)-4-(iodoacetamido)butane (AsIB), N-[4-(p-azidosalicylamido)butyl]-3′-(2′-pyridyldithio) propionamide (APDP), benzophenone-4-iodoacetamide, p-azidobenzoyl hydrazide (ABH), 4-(p-azidosalicylamido)butylamine (AsBA), or p-azidophenyl glyoxal (APG). In some embodiments, the linker comprises a cleavable linker, optionally comprising a dipeptide linker. In some embodiments, the dipeptide linker comprises Val-Cit, Phe-Lys, Val-Ala, or Val-Lys. In some embodiments, the linker comprises a non-cleavable linker. In some embodiments, the linker comprises a maleimide group, optionally comprising maleimidocaproyl (mc), succinimidyl-4-(N-maleimidomethyl)cyclohexane-1-carboxylate (sMCC), or sulfosuccinimidyl-4-(N-maleimidomethyl)cyclohexane-1-carboxylate (sulfo-sMCC). In some embodiments, the linker further comprises a spacer. In some embodiments, the spacer comprises p-aminobenzyl alcohol (PAB), p-aminobenzyoxycarbonyl (PABC), a derivative, or an analog thereof. In some embodiments, the conjugating moiety is capable of extending the serum half-life of the IL-2 conjugate. In some embodiments, the additional conjugating moiety is capable of extending the serum half-life of the IL-2 conjugate. In some embodiments, the IL-2 form suitable for use in the invention is a fragment of any of the IL-2 forms described herein. In some embodiments, the IL-2 form suitable for use in the invention is pegylated as disclosed in U.S. Patent Application Publication No. US 2020/0181220 A1 and U.S. Patent Application Publication No. US 2020/0330601 A1. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 80% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 polypeptide comprises an N-terminal deletion of one residue relative to SEQ ID NO:5. In some embodiments, the IL-2 form suitable for use in the invention lacks IL-2R alpha chain engagement but retains normal binding to the intermediate affinity IL-2R beta-gamma signaling complex. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 90% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 95% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 98% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:570.

In some embodiments, an IL-2 form suitable for use in the invention is nemvaleukin alfa, also known as ALKS-4230 (SEQ ID NO:571), which is available from Alkermes, Inc. Nemvaleukin alfa is also known as human interleukin 2 fragment (1-59), variant (Cys¹²⁵>Ser⁵¹), fused via peptidyl linker (⁶⁰GG⁶¹) to human interleukin 2 fragment (62-132), fused via peptidyl linker (¹³³GSGGGS¹³⁸) to human interleukin 2 receptor α-chain fragment (139-303), produced in Chinese hamster ovary (CHO) cells, glycosylated; human interleukin 2 (IL-2) (75-133)-peptide [Cys¹²⁵ (51)>Ser]-mutant (1-59), fused via a G₂ peptide linker (60-61) to human interleukin 2 (IL-2) (4-74)-peptide (62-132) and via a GSG₃S peptide linker (133-138) to human interleukin 2 receptor α-chain (IL2R subunit alpha, IL2Ra, IL2RA) (1-165)-peptide (139-303), produced in Chinese hamster ovary (CHO) cells, glycoform alfa. The amino acid sequence of nemvaleukin alfa is given in SEQ ID NO:571. In some embodiments, nemvaleukin alfa exhibits the following post-translational modifications: disulfide bridges at positions: 31-116, 141-285, 184-242, 269-301, 166-197 or 166-199, 168-199 or 168-197 (using the numbering in SEQ ID NO: 571), and glycosylation sites at positions: N187, N206, T212 using the numbering in SEQ ID NO:571. The preparation and properties of nemvaleukin alfa, as well as additional alternative forms of IL-2 suitable for use in the invention, is described in U.S. Patent Application Publication No. US 2021/0038684 A1 and U.S. Pat. No. 10,183,979, the disclosures of which are incorporated by reference herein. In some embodiments, an IL-2 form suitable for use in the invention is a protein having at least 80%, at least 90%, at least 95%, or at least 90% sequence identity to SEQ ID NO: 571. In some embodiments, an IL-2 form suitable for use in the invention has the amino acid sequence given in SEQ ID NO: 571 or conservative amino acid substitutions thereof. In some embodiments, an IL-2 form suitable for use in the invention is a fusion protein comprising amino acids 24-452 of SEQ ID NO:572, or variants, fragments, or derivatives thereof. In some embodiments, an IL-2 form suitable for use in the invention is a fusion protein comprising an amino acid sequence having at least 80%, at least 90%, at least 95%, or at least 90% sequence identity to amino acids 24-452 of SEQ ID NO: 572, or variants, fragments, or derivatives thereof. Other IL-2 forms suitable for use in the present invention are described in U.S. Pat. No. 10,183,979, the disclosures of which are incorporated by reference herein. Optionally, in some embodiments, an IL-2 form suitable for use in the invention is a fusion protein comprising a first fusion partner that is linked to a second fusion partner by a mucin domain polypeptide linker, wherein the first fusion partner is IL-1Rα or a protein having at least 98% amino acid sequence identity to IL-1Rα and having the receptor antagonist activity of IL-Rα, and wherein the second fusion partner comprises all or a portion of an immunoglobulin comprising an Fc region, wherein the mucin domain polypeptide linker comprises SEQ ID NO:573 or an amino acid sequence having at least 90% sequence identity to SEQ ID NO:573 and wherein the half-life of the fusion protein is improved as compared to a fusion of the first fusion partner to the second fusion partner in the absence of the mucin domain polypeptide linker.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH IRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRCLISNINV IVLELKGSET TFMCEYADET ATIVEFLNRW | 120 |
| | ITFSQSIIST LT | 132 |
| SEQ ID NO: 5 | MHKCDITLQE IIKTLNSLTE CKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTI ENFLERLKTI | 120 |
| human IL-4 | MREKYSKCSS | 130 |
| (rhIL-4) | | |
| SEQ ID NO: 6 | MDCDIEGKDG KQYESVLMVS IDCLLDSMKE IGSNCLNNEF NFFKRHICDA NKEGMFLFRA | 60 |
| recombinant | ARKLRQFLKM NSTGDFDLHL IKVSEGTTIL LNCTGQVKGR KPAALGEAQP TKSLEENKSI | 120 |
| human IL-7 | KEQKKLNDLC FLKRLLQEIK TCWNKILMGT KEH | 153 |
| (rhIL-7) | | |
| SEQ ID NO: 7 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 8 | MQDRHMIRMR QLIDIVDQLK NYVNDLVPEF LPAPEDVETN CEWSAFSCFC KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | HLSSRTHGSE DS | 132 |
| (rhIL-21) | | |
| SEQ ID NO: 570 | APTSSSTKKT QLQLEHLLLD LQMILNGINN YKNPKLTRML TFKFYMPKKA TELKHLQCLE | 60 |
| IL-2 form | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 571 | SKNFHLRPRD LISNINVIVL ELKGSETTFM CEYADETATI VEFLNRWITF SQSIISTLTG | 60 |
| IL-2 form | GSSSTKKTQL QLEHLLLDLQ MIINGINNYK NPKLTRMLTF KFYMPKKATE LKHLQCLEEE | 120 |
| | LKPLEEVLNL AQGSGGGSEL CDDDPPEIPH ATFKAMAYKE GTMLNCECKR GFRRIKSGSL | 180 |
| | YMLCTGNSSH SSWDNQCQCT SSATRNTTKQ VTPQPEEQKE RKITEMQSPM QPVDQASLPG | 240 |
| | HCREPPPWEN EATERIYHFV VGCMVYYQCV QGYRALHRGP AESVCKMTHG KTRWTQPQLI | 300 |
| | CTG | 303 |
| SEQ ID NO: 572 | MDAMKRGLCC VLLLCGAVFV SARRPSGRKS SKMQAFRIWD VNQKTFYLRN NQLVAGYLQG | 60 |
| IL-2 form | PNVNLEEKID VVPIEPHALF LGIHGGKMCL SCVKSGDETR LQLEAVNITD LSENRKQDKR | 120 |
| | FAFIRSDSGP TTSFESAACP GWFLCTAMEA DQPVSLTNMP DEGVMVTKFY FQEDESGSGG | 130 |
| | ASSESSASSD GPHPVITESR ASSESSASSD GPHPVITESR EPKSSDKTHT CPPCPAPELL | 240 |
| | GGPSVFLFPP KPKDTLMISR TPEVTCVVVD VSHEDPEVKF NWYVDGVEVH NAKTKPREEQ | 300 |
| | YNSTYRVVSV LTVLHQDWLN GKEYKCKVSN KALPAPIEKT ISKAKGQPRE PQVYTLPPSR | 360 |
| | EEMTKNQVSL TCLVKGFYPS DIAVEWESNG QPENNYKTT PPVLDSDGSF FLYSKLTVDK | 420 |
| | SRWQQGNVFS CSVMHEALHN HYTQKSLSLS PGK | 453 |
| SEQ ID NO: 573 | SESSASSDGP HPVITP | 16 |
| mucin domain | | |
| polypeptide | | |

In some embodiments, an IL-2 form suitable for use in the invention includes an antibody cytokine engrafted protein that comprises a heavy chain variable region (VH), comprising complementarity determining regions HCDR1, HCDR2, HCDR3; a light chain variable region (VL), comprising LCDR1, LCDR2, LCDR3; and an IL-2 molecule or a fragment thereof engrafted into a CDR of the Vii or the VL, wherein the antibody cytokine engrafted protein preferentially expands T effector cells over regulatory T cells. In some embodiments, the antibody cytokine engrafted protein comprises a heavy chain variable region (VII), comprising complementarity determining regions HCDR1, HCDR2, HCDR3; a light chain variable region (VL), comprising LCDR1, LCDR2, LCDR3; and an IL-2 molecule or a fragment thereof engrafted into a CDR of the VH or the VL, wherein the IL-2 molecule is a mutein, and wherein the antibody cytokine engrafted protein preferentially expands T effector cells over regulatory T cells. In some embodiments, the IL-2 regimen comprises administration of an antibody described in U.S. Patent Application Publication No. US 2020/0270334 A1, the disclosures of which are incorporated by reference herein. In some embodiments, the antibody cytokine engrafted protein comprises a heavy chain variable region (VH), comprising complementarily determining regions HCDR1, HCDR2, HCDR3; a light chain variable region (VL), comprising LCDR1, LCDR2, LCDR3; and an IL-2 molecule or a fragment thereof engrafted into a CDR of the VH or the VL, wherein the IL-2 molecule is a mutein, wherein the antibody cytokine engrafted protein preferentially expands T effector cells over regulatory T cells, and wherein the antibody further comprises an IgG class heavy chain and an IgG class light chain selected from the group consisting of: a IgG class light chain comprising SEQ ID NO:569 and a IgG class heavy chain comprising SEQ ID NO:568; a IgG class light chain comprising SEQ ID NO:567 and a IgG class heavy chain comprising SEQ ID NO:559; a IgG class light chain comprising SEQ ID NO:569 and a IgG class heavy chain comprising SEQ ID NO:559; and a IgG class light chain comprising SEQ ID NO:37 and a IgG class heavy chain comprising SEQ ID NO:568.

In some embodiments, an IL-2 molecule or a fragment thereof is engrafted into HCDR1 of the VH, wherein the IL-2 molecule is a mutein. In some embodiments, an IL-2 molecule or a fragment thereof is engrafted into HCDR2 of the VH, wherein the IL-2 molecule is a mutein. In some embodiments, an IL-2 molecule or a fragment thereof is engrafted into HCDR3 of the Vu, wherein the IL-2 molecule is a mutein. In some embodiments, an IL-2 molecule or a fragment thereof is engrafted into LCDR1 of the VL, wherein the IL-2 molecule is a mutein. In some embodiments, an IL-2 molecule or a fragment thereof is engrafted into LCDR2 of the VL, wherein the IL-2 molecule is a mutein. In some embodiments, an IL-2 molecule or a fragment thereof is engrafted into LCDR3 of the VL, wherein the IL-2 molecule is a mutein.

The insertion of the IL-2 molecule can be at or near the N-terminal region of the CDR, in the middle region of the CDR or at or near the C-terminal region of the CDR. In some embodiments, the antibody cytokine engrafted protein comprises an IL-2 molecule incorporated into a CDR, wherein the IL2 sequence does not frameshift the CDR sequence. In some embodiments, the antibody cytokine engrafted protein comprises an IL-2 molecule incorporated into a CDR, wherein the IL-2 sequence replaces all or part of a CDR sequence. The replacement by the IL-2 molecule can be the N-terminal region of the CDR, in the middle region of the CDR or at or near the C-terminal region the CDR. A replacement by the IL-2 molecule can be as few as one or two amino acids of a CDR sequence, or the entire CDR sequences.

In some embodiments, an IL-2 molecule is engrafted directly into a CDR without a peptide linker, with no additional amino acids between the CDR sequence and the IL-2 sequence. In some embodiments, an IL-2 molecule is engrafted indirectly into a CDR with a peptide linker, with one or more additional amino acids between the CDR sequence and the IL-2 sequence.

In some embodiments, the IL-2 molecule described herein is an IL-2 mutein. In some instances, the IL-2 mutein comprising an R67A substitution. In some embodiments, the IL-2 mutein comprises the amino acid sequence SEQ ID NO:544 or SEQ ID NO:545. In some embodiments, the IL-2 mutein comprises an amino acid sequence in Table 1 in U.S. Patent Application Publication No. US 2020/0270334 A1, the disclosure of which is incorporated by reference herein.

In some embodiments, the antibody cytokine engrafted protein comprises an HCDR1 selected from the group consisting of SEQ ID NO:546, SEQ ID NO:549, SEQ ID NO:552 and SEQ ID NO:555. In some embodiments, the antibody cytokine engrafted protein comprises an HCDR1 selected from the group consisting of SEQ ID NO:7, SEQ ID NO:10, SEQ ID NO:543 and SEQ ID NO:546. In some embodiments, the antibody cytokine engrafted protein comprises an HCDR1 selected from the group consisting of HCDR2 selected from the group consisting of SEQ ID NO:547, SEQ ID NO:550, SEQ ID NO:553, and SEQ ID NO:556. In some embodiments, the antibody cytokine engrafted protein comprises an HCDR3 selected from the group consisting of SEQ ID NO:548, SEQ ID NO:551, SEQ ID NO:554, and SEQ ID NO:557. In some embodiments, the antibody cytokine engrafted protein comprises a V_{H} region comprising the amino acid sequence of SEQ ID NO:558. In some embodiments, the antibody cytokine engrafted protein comprises a heavy chain comprising the amino acid sequence of SEQ ID NO:559. In some embodiments, the antibody cytokine engrafted protein comprises a VL region comprising the amino acid sequence of SEQ ID NO:566. In some embodiments, the antibody cytokine engrafted protein comprises a light chain comprising the amino acid sequence of SEQ ID NO:567. In some embodiments, the antibody cytokine engrafted protein comprises a VII region comprising the amino acid sequence of SEQ ID NO:28 and a VL region comprising the amino acid sequence of SEQ ID NO:566. In some embodiments, the antibody cytokine engrafted protein comprises a heavy chain region comprising the amino acid sequence of SEQ ID NO:559 and a light chain region comprising the amino acid sequence of SEQ ID NO:567. In some embodiments, the antibody cytokine engrafted protein comprises a heavy chain region comprising the amino acid sequence of SEQ ID NO:559 and a light chain region comprising the amino acid sequence of SEQ ID NO:569. In some embodiments, the antibody cytokine engrafted protein comprises a heavy chain region comprising the amino acid sequence of SEQ ID NO:568 and a light chain region comprising the amino acid sequence of SEQ ID NO:567. In some embodiments, the antibody cytokine engrafted protein comprises a heavy chain region comprising the amino acid sequence of SEQ ID NO:568 and a light chain region comprising the amino acid sequence of SEQ ID NO:569. In some embodiments, the antibody cytokine engrafted protein comprises IgG.IL2F71A.H1 or IgG.IL2R67A.H1 of U.S. Patent Application Publication No. 2020/0270334 A1, or variants, derivatives, or fragments thereof, or conservative amino acid substitutions thereof, or proteins with at least 80%, at least 90%, at least 95%, or at least 98% sequence identity thereto. In some embodiments, the antibody components of the antibody cytokine engrafted protein described herein comprise immunoglobulin sequences, framework sequences, or CDR sequences of palivizumab. In some embodiments, the antibody cytokine engrafted protein described herein has a longer serum half-life that a wild-type IL-2 molecule such as, but not limited to, aldesleukin or a comparable molecule.

TABLE 3
Sequences of exemplary palivizumab antibody-IL-2 engrafted proteins

| Identifier | | | |
|---|---|---|---|
| US | SEQ ID | | |
| 2020/0270334 | NO: | Sequence | |
| SEQ ID NO: 2 | SEQ ID | MYRMQLLSCI ALSLALVTNS APTSSSTKKT QLQLEHLLLD LQMILNGINN | 50 |
| IL-2 | NO: 543 | YKNPKLTRML TFKFYMPKKA TELKHLQCZE EELKPLEEVL NLAQSKNFHL | 100 |
| | IL-2 | RPRDLISNIN VIVLELKGSE TTFMCEYADE TATIVEFLNR WITFCQSIIS | 150 |
| | | TLT | 153 |
| SEQ ID NO: 4 | SEQ ID | APTSSSTKKT QLQLEHLLLD LQMILNGINN YKNPKLTAML TEKFYMPKKA | 50 |
| IL-2 mutein | NO: 544 | TELKHLQCLE EELKPLEEVL NLAQSKNFHL RPRDLISNIN VIVLELKGSE | 100 |
| | IL-2 | TTFMCEYADE TATIVEFLNR WITFCQSIIS TLT | 133 |
| | mutein | | |
| SEQ ID NO: 6 | SEQ ID | APTSSSTKKT QLQLEHLLLD LQMILNGINN YKNPKLTRML TAKFYMPKKA | 50 |
| IL-2 mutein | NO: 545 | TELKHLQCLE EE_KPLEEVL NLAQSKNFHL RPRDLISNIN VIVLELKGSE | 100 |
| | IL-2 | TTFMCEYADE TATIVEFLNR WITFCQSIIS TLT | 133 |
| | mutein | | |
| IgG.IL2R67A.H1 | IgG.IL2R67A.H1 | | |
| SEQ ID NO: 7 | SEQ ID | GFSLAPTSSS TKKTQLQLEH LLLDLQMIEN GINNYKNPKL TAMLTFKFYM | 50 |
| HCDR1_IL-2 | NO: 546 | PKKATELKHL QCLEEELKPL EEVLNLAQSK NFHLRPRDLI SNINVIVLEL | 100 |
| | HCDR1_IL-2 | KGSETTFMCE YADETATIVE FLNRWITFCQ SIISTLTSTS GMSVG | 145 |
| SEQ ID NO: 8 | SEQ ID | DIWWDDKKDY NPSLKS | 16 |
| HCDR2 | NO: 547 | | |
| | HCDR2 | | |
| SEQ ID NO: 9 | SEQ ID | SMITNWYFDV | 10 |
| HCDR3 | NO: 548 | | |
| | HCDR3 | | |
| SEQ ID NO: 10 | SEQ ID | TELKHLQCLE EELKPLEEVL NLAQSKNFHL RPRDLISNIN VIVLELKGSE | 100 |
| HCDR1_IL-2 | NO: 549 | TTFMCEYADE TATIVEFLNR WITFCQSIIS TLTSTSGMSV G | 141 |
| kabat | HCDR1_IL-2 | | |
| | kabat | | |
| SEQ ID NO: 11 | SEQ ID | DIWWDDKKDY NPSLKS | 16 |
| HCDR2 kabat | NO: 550 | | |
| | HCDR2 | | |
| | kabat | | |
| SEQ ID NO: 12 | SEQ ID | SMITNWYFDV | 10 |
| HCDR3 kabat | NO: 551 | | |
| | HCDR3 | | |
| | kabat | | |
| SEQ ID NO: 13 | SEQ ID | GFSLAPTSSS TKKTQLQLEH LLLDLQMIEN GINNYKNPKL TAMLTFKFYM | 50 |
| HCDR1_IL-2 | NO: 552 | PKKATELKHL QCLEEELKPL EEVLNLAQSK NFHLRPRDLI SNINVIVLEL | 100 |
| clothia | HCDR1_IL-2 | KGSETTFMCE YADETATIVE FLNRWITFCQ SIISTLTSTS GM | 142 |
| | clothia | | |
| SEQ ID NO: 14 | SEQ ID | WWDDK | 5 |
| HCDR2 | NO: 553 | | |
| clothia | HCDR2 | | |
| | clothia | | |
| SEQ ID NO: 15 | SEQ ID | SMITNWYFDV | 10 |
| HCDR3 | NO: 554 | | |
| clothia | HCDR3 | | |
| | clothia | | |
| SEQ ID NO: 16 | SEQ ID | GFSLAPTSSS TKKTQLQLEH LLLDLQMIEN GINNYKNPKL TAMLTFKFYM | 50 |
| HCDR1_IL-2 | NO: 555 | PKKATELKHL QCLEEELKPL EEVLNLAQSK NFHLRPRDLI SNINVIVLEL | 100 |
| IMGT | HCDR1_IL-2 | KGSETTFMCE YADETATIVE FLNRWITFCQ SIISTLTSTS GMS | 143 |
| | IMGT | | |
| SEQ ID NO: 17 | SEQ ID | IWWDDKK | 7 |
| HCDR2 IMGT | NO: 556 | | |
| | HCDR2 | | |
| | IMGT | | |
| SEQ ID NO: 18 | SEQ ID | ARSMITNWYF DV | 12 |
| HCDR3 IMGT | NO: 557 | | |
| | HCDR3 | | |
| | IMGT | | |
| SEQ ID NO: 19 | SEQ ID | QVTLRESGPA LVKPTQTLTL TCTFSGFSLA PTSSSTKKTQ LQLEHLLLDL | 50 |
| VH | NO: 558 | QMILNGINNY KNPKLTAMLT FKFYMPKKAT ELKHLQCLEE ELKPLEEVLN | 100 |
| | VH | LAQSKNFHLR PRDLISNINV IVLELKGSET TEMCEYADET ATIVEFLNRW | 150 |
| | | ITFCQSIIST LTSTSGMSVG WIRQPPGKAL EWLADIWWDD KKDYNPSLKS | 200 |
| | | RLTISKDTSK NQVVLKVTNM DPADTATYYC ARSMITNWYF DVWGAGTTVT | 250 |
| | | VSS | 253 |
| SEQ ID NO: 21 | SEQ ID | QMILNGINNY KNPKLTAMLT FKFYMPKKATELKHLQCLEE ELKPLEEVLN | 100 |
| Heavy chain | NO: 559 | LAQSKNFHLR PRDLISNINV IVLELKGSET TFMCEYADET ATIVEFLNRW | 150 |
| | Heavy | ITFCQSIIST LTSTSGMSVG WIRQPPGKAL EWLADIWWDD KKDYNPSLKS | 200 |
| | chain | RLTISKDTSK NQVVLKVTNM DPADTATYYC ARSMITNWYF DVWGAGTTVT | 250 |
| | | VSSASTKGPS VFPLAPSSKS TSGGTAALGC LVKDYFPEPV TVSWNSGALT | 300 |
| | | SGVHTFPAVL QSSGLYSLSS VVTVPSSSLG TQTYICNVNH KPSNTKVDKR | 350 |
| | | VEPKSCDKTH TCPPCPAPEL LGGPSVFLFP PKPKDTLMIS RTPEVTCVVV | 400 |
| | | AVSHEDPEVK FNWYVDGVEV HNAKTKPREE QYNSTYRVVS VLTVLHQDWL | 450 |
| | | NGKEYKCKVS NKALAAPIEK TISKAKGQPR EPQVYTLPPS REEMTKNQVS | 500 |
| | | LTCLVKGFYP SDIAVEWESN GQPENNYKTT PPVLDSDGSF FLYSKLTVDK | 550 |
| | | SRWQQGNVFS CSVMHEALHN HYTQKSLSES PGK | 583 |
| SEQ ID NO: 26 | SEQ ID | KAQLSVGYMH | 10 |
| LCDR1 kabat | NO: 560 | | |
| | LCDR1 | | |
| | kabat | | |
| SEQ ID NO: 27 | SEQ ID | DTSKLAS | 7 |
| LCDR2 kabat | NO: 561 | | |
| | LCDR2 | | |
| | kabat | | |
| SEQ ID NO: 28 | SEQ ID | FQGSGYPFT | 9 |
| LCDR3 kabat | NO: 562 | | |
| | LCDR3 | | |
| | kabat | | |
| SEQ ID NO: 29 | SEQ ID | QLSVGY | 6 |
| LCDR1 | NO: 563 | | |
| chothia | LCDR1 | | |
| | chothia | | |
| SEQ ID NO: 30 | SEQ ID | DTS | 3 |
| LCDR2 | NO: 564 | | |
| chothia | LCDR2 | | |
| | chothia | | |
| SEQ ID NO: 31 | SEQ ID | GSGYPF | 6 |
| LCDR3 | NO: 565 | | |
| chothia | LCDR3 | | |
| | chothia | | |
| SEQ ID NO: 35 | SEQ ID | DIQMTQSPST LSASVGDRVT ITCKAQLSVG YMHWYQQKPG KAPKLLIYDT | 50 |
| VL | NO: 566 | SKLASGVPSR FSGSGSGTEF TLTISSLQPD DFATYYCFQG SGYPFTFGGG | 100 |
| | VL | TKLEIK | 106 |
| SEQ ID NO: 37 | SEQ ID | DIQMTQSPST LSASVGDRVT ITCKAQLSVG YMHWYQQKPG KAPKLLIYDT | 50 |
| Light chain | NO: 567 | SKLASGVPSR FSGSGSGTEF TLTISSLQPD DFATYYCFQG SGYPFTFGGG | 100 |
| | Light | TKLEIKRTVA APSVFIFPPS DEQLKSGTAS VVCLLNNFYP REAKVQWKVD | 150 |
| | chain | NALQSGNSQE SVTEQDSKDS TYSLSSTLTL SKADYEKHKV YACEVTHQGL | 200 |
| | | SSPVTKSFNR GEC | 213 |
| SEQ ID NO: 53 | SEQ ID | QVTLRESGPA LVKPTQTLTL TCTFSGFSLA PTSSSTKKTQ LQLEHLLLDL | 50 |
| Light chain | NO: 568 | QMILNGINNY KNPKLTRMLT AKFYMPKKAT ELKHLQCLEE ELKPLEEVLN | 100 |
| | Light | LAQSKNFHLR PRDLISNINV IVLELKGSET TFMCEYADET ATIVEFLNRW | 150 |
| | chain | ITFCQSIIST LTSTSGMSVG WIRQP?GKAL EWLADIWWDD KKDYNPSLKS | 200 |
| | | RLTISKDTSK NQVVLKVTNM DPADTATYYC ARSMITNWYF DVWGAGTTVT | 250 |
| | | VSSASTKGPS VFPLAPSSKS TSGGTAALGC LVKDYFPEPV TVSWNSGALT | 300 |
| | | SGVHTFPAVL QSSGLYSLSS VVTVPSSSLG TQTYICNVNH KPSNTKVDKR | 350 |
| | | VEPKSCDKTH TCPPCPAPEL LGGPSVFLFP PKPKDTLMIS RTPEVTCVVV | 400 |
| | | AVSHEDPEVK FNWYVDGVEV HNAKTKPREE QYNSTYRVVS VLTVLHQDWL | 450 |
| | | NGKEYKCKVS NKALAAPIEK TISKAKGQPR EPQVYTLPPS REEMTKNQVS | 500 |
| | | LTCLVKGFYP SDIAVEWESN GQPENNYKTT PPVLDSDGSF FLYSKLTVDK | 550 |
| | | SRWQQGNVFS CSVMHEALHN HYTQKSLSES PGK | 583 |
| SEQ ID NO: 69 | SEQ ID | DIQMTQSPST LSASVGDRVT ITCKAQLSVG YMHWYQQKPG KAPKLLIYDT | 50 |
| Light chain | NO: 569 | SKLASGVPSR FSGSGSGTEF TLTISSLQPD DFATYYCFQG SGYPFTFGGG | 100 |
| | Light | TKLEIKRTVA APSVFIFPPS DEQLKSGTAS VVCLLNNFYP REAKVQWKVD | 150 |
| | chain | NALQSGNSQE SVTEQDSKDS TYSLSSTLTL SKADYEKHKV YACEVTHQGL | 200 |
| | | SSPVTKSFNR GEC | 213 |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, N.J., USA (Cat. No. CYT-211) and ThermoFisher Scientific, Inc., Waltham, Mass., USA (human IL-15 recombinant protein, Cat. No. Gibco CTP0043). The amino acid sequence of recombinant human IL-4 suitable for use in the invention is given in Table 2 (SEQ ID NO:5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, NJ, USA (Cat. No. CYT-254) and ThermoFisher Scientific, Inc., Waltham, Mass., USA (human IL-15 recombinant protein, Cat. No. Gibco PHC0071). The amino acid sequence of recombinant human IL-7 suitable for use in the invention is given in Table 2 (SEQ ID NO:6).

The term “IL-15” (also referred to herein as “IL15”) refers to the T cell growth factor known as interleukin-15, and includes all forms of IL-2 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). The amino acid sequence of recombinant human IL-15 suitable for use in the invention is given in Table 2 (SEQ ID NO:7).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, NJ, USA (Cat. No. CYT-408-b) and ThermoFisher Scientific, Inc., Waltham, Mass., USA (human IL-21 recombinant protein, Cat. No. 14-8219-80). The amino acid sequence of recombinant human IL-21 suitable for use in the invention is given in Table 2 (SEQ ID NO:8).

When “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the tumor infiltrating lymphocytes (e.g. secondary TILs or genetically modified cytotoxic lymphocytes) described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. Tumor infiltrating lymphocytes (including in some cases, genetically modified cytotoxic lymphocytes) compositions may also be administered multiple times at these dosages. The tumor infiltrating lymphocytes (including in some cases, genetically) can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

The term “hematological malignancy,” “hematologic malignancy” or terms of correlative meaning refer to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “solid tumor” refers to an abnormal mass of tissue that usually does not contain cysts or liquid areas. Solid tumors may be benign or malignant. The term “solid tumor cancer refers to malignant, neoplastic, or cancerous solid tumors. Solid tumor cancers include, but are not limited to, sarcomas, carcinomas, and lymphomas, such as cancers of the lung, breast, prostate, colon, rectum, and bladder. The tissue structure of solid tumors includes interdependent tissue compartments including the parenchyma (cancer cells) and the supporting stromal cells in which the cancer cells are dispersed and which may provide a supporting microenvironment.

The term “liquid tumor” refers to an abnormal mass of cells that is fluid in nature. Liquid tumor cancers include, but are not limited to, leukemias, myelomas, and lymphomas, as well as other hematological malignancies. TILs obtained from liquid tumors may also be referred to herein as marrow infiltrating lymphocytes (MILs). TILs obtained from liquid tumors, including liquid tumors circulating in peripheral blood, may also be referred to herein as PBLs. The terms MIL, TIL, and PBL are used interchangeably herein and differ only based on the tissue type from which the cells are derived.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

In some embodiments, the invention includes a method of treating a cancer with a population of TILs, wherein a patient is pre-treated with non-myeloablative chemotherapy prior to an infusion of TILs according to the invention. In some embodiments, the population of TILs may be provided wherein a patient is pre-treated with nonmyeloablative chemotherapy prior to an infusion of TILs according to the present invention. In some embodiments, the non-myeloablative chemotherapy is cyclophosphamide 60 mg/kg/d for 2 days (days 27 and 26 prior to TIL infusion) and fludarabine 25 mg/m2/d for 5 days (days 27 to 23 prior to TIL infusion). In some embodiments, the non-myeloablative chemotherapy is cyclophosphamide 60 mg/kg/d for 2 days (days 27 and 26 prior to TIL infusion) and fludarabine 25 mg/m2/d for 3 days (days 27 to 25 prior to TIL infusion). In some embodiments, the non-myeloablative chemotherapy is cyclophosphamide 60 mg/kg/d for 2 days (days 27 and 26 prior to TIL infusion) followed by fludarabine 25 mg/m2/d for 3 days (days 25 to 23 prior to TIL infusion). In some embodiments, after non-myeloablative chemotherapy and TIL infusion (at day 0) according to the invention, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the rTILs of the invention.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, at least one potassium channel agonist in combination with a plurality of TILs) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

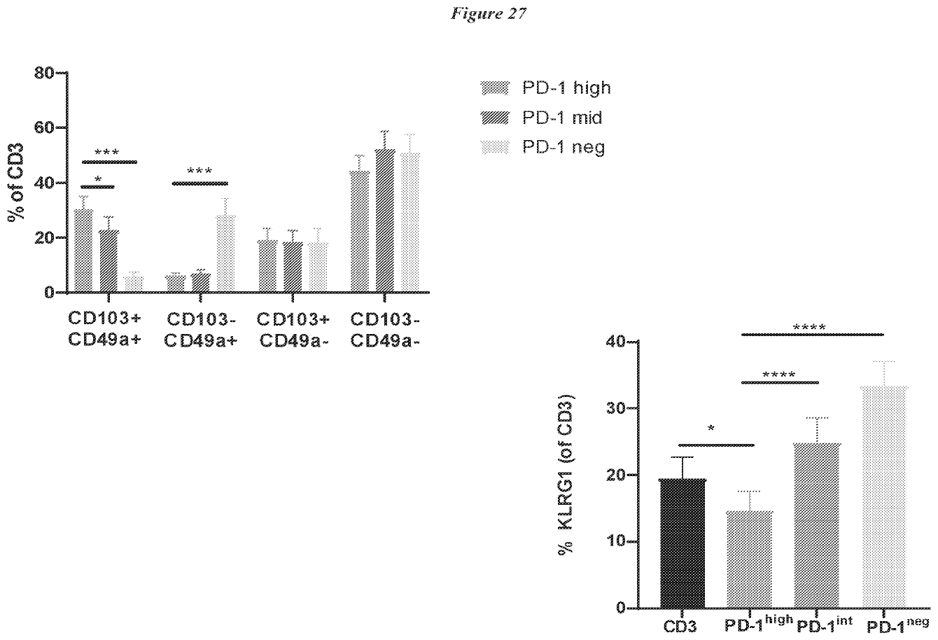

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly obtained” or “freshly isolated”), and “secondary TILs” are any TIL cell populations that have been expanded or proliferated as discussed herein, including, but not limited to bulk TILs, expanded TILs (“REP TILs”) as well as “reREP TILs” as discussed herein. reREP TILs can include for example second expansion TILs or second additional expansion TILs (such as, for example, those described in Step D of FIG. 27, including TILs referred to as reREP TILs).

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally, and alternatively, TILs can be functionally defined by their ability to infiltrate solid tumors upon reintroduction into a patient. TILs may further be characterized by potency—for example, TILs may be considered potent if, for example, interferon (IFN) release is greater than about 50 pg/mL, greater than about 100 pg/mL, greater than about 150 pg/mL, or greater than about 200 pg/mL. TILs may be considered potent if, for example, interferon (IFNγ) release is greater than about 50 pg/mL, greater than about 100 pg/mL, greater than about 150 pg/mL, or greater than about 200 pg/mL, greater than about 300 pg/mL, greater than about 400 pg/mL, greater than about 500 pg/mL, greater than about 600 pg/mL, greater than about 700 pg/mL, greater than about 800 pg/mL, greater than about 900 pg/mL, greater than about 1000 pg/mL.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in therapeutic compositions of the invention is contemplated. Additional active pharmaceutical ingredients, such as other drugs, can also be incorporated into the described compositions and methods.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of”

The term “PD-1 high” or “PD-1high” or “PD-1^{high}” refers to a high level of PD-1 protein expression by a cell such as, but not limited to, a tumor infiltrating lymphocyte or a T cell relative to a control cell from a healthy subject. In some embodiments, the level of PD-1 expression is determined using a standard method known to those skilled in the art for measuring protein levels present on a cell such as flow cytometry, fluorescence activated cell sorting (FACS), immunocytochemistry, and the like. In some cases, a PD-1 high TIL expresses a greater level of PD-1 compared to an immune cell from a healthy subject. In some cases, a population of PD-1 high TILs expresses a greater level of PD-1 compared to a population of immune cells (e.g., peripheral blood mononuclear cells) from a healthy subject or a group of healthy subjects. PD-1high cells can be referred to as PD-1 bright cells.

The term “PD-1 intermediate” or “PD-lint” or PD-1^{int}” refers to an intermediate or moderate level of PD-1 protein expression by a cell such as, but not limited to, a tumor infiltrating lymphocyte or a T cell relative to a control cell from a healthy subject. For instance, a PD-lint T cell expresses PD-1 protein at a level or range that is similar to or substantially equivalent to the highest range of PD-1 protein expressed by a control cell (e.g., peripheral blood mononuclear cell) from a healthy subject. In other words, a PD-lint TIL has a PD-1 expression level that is similar to or substantially equivalent to a background level of PD-1 expression by a control immune cell from a healthy subject. PD-lint cells can be referred to as PD-1 dim cells. One skilled in the art recognizes that a PD-1positive TIL can be a PD-1high TIL or a PD-lint TIL.

The term “PD-1 negative” or “PD-1neg” or “PD-1^{neg}” refers to negative or low level of PD-1 protein expression by a cell such as, but not limited to, a tumor infiltrating lymphocyte or a T cell relative to a control cell from a healthy subject. For instance, a PD-1neg T cell does not expresses PD-1 protein. In some instances, a PD-1neg T cell expresses PD-1 protein at a level that is similar to or substantially equivalent to the lowest level of PD-1 protein expressed by a control cell (e.g., peripheral blood mononuclear cell) from a healthy subject. PD-1neg lymphocytes can express PD-1 at the same level or range as a majority of lymphocytes in a control population.

PD-1high, PD-lint, and PD-1neg TILs are distinct and different subsets of TILs expanded ex vivo according to the methods described herein. In some embodiments, a population of ex vivo expanded TILs comprises PD-1high TILs, PD-lint TILs, and PD-1neg TILs.

II. TIL Manufacturing Processes (Embodiments of Gen 3 Processes, Optionally Including Defined Media)

In addition to the methods described herein, International Application No. PCT/US2019/059716 is incorporated by reference herein in its entirety for all purposes. Without being limited to any particular theory, it is believed that the priming first expansion that primes an activation of T cells followed by the rapid second expansion that boosts the activation of T cells as described in the methods of the invention allows the preparation of expanded T cells that retain a “younger” phenotype, and as such the expanded T cells of the invention are expected to exhibit greater cytotoxicity against cancer cells than T cells expanded by other methods. In particular, it is believed that an activation of T cells that is primed by exposure to an anti-CD3 antibody (e.g. OKT-3), IL-2 and optionally antigen-presenting cells (APCs) and then boosted by subsequent exposure to additional anti-CD-3 antibody (e.g. OKT-3), IL-2 and APCs as taught by the methods of the invention limits or avoids the maturation of T cells in culture, yielding a population of T cells with a less mature phenotype, which T cells are less exhausted by expansion in culture and exhibit greater cytotoxicity against cancer cells. In some embodiments, the step of rapid second expansion is split into a plurality of steps to achieve a scaling up of the culture by: (a) performing the rapid second expansion by culturing T cells in a small scale culture in a first container, e.g., a G-REX 100MCS container, for a period of about 3 to 4 days, and then (b) effecting the transfer of the T cells in the small scale culture to a second container larger than the first container, e.g., a G-REX 500MCS container, and culturing the T cells from the small scale culture in a larger scale culture in the second container for a period of about 4 to 7 days. In some embodiments, the step of rapid expansion is split into a plurality of steps to achieve a scaling out of the culture by: (a) performing the rapid second expansion by culturing T cells in a first small scale culture in a first container, e.g., a G-REX 100MCS container, for a period of about 3 to 4 days, and then (b) effecting the transfer and apportioning of the T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the T cells from first small scale culture transferred to such second container is cultured in a second small scale culture for a period of about 4 to 7 days. In some embodiments, the step of rapid expansion is split into a plurality of steps to achieve a scaling out and scaling up of the culture by: (a) performing the rapid second expansion by culturing T cells in a small scale culture in a first container, e.g., a G-REX 100MCS container, for a period of about 3 to 4 days, and then (b) effecting the transfer and apportioning of the T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the T cells from the small scale culture transferred to such second container is cultured in a larger scale culture for a period of about 4 to 7 days. In some embodiments, the step of rapid expansion is split into a plurality of steps to achieve a scaling out and scaling up of the culture by: (a) performing the rapid second expansion by culturing T cells in a small scale culture in a first container, e.g., a G-REX 100MCS container, for a period of about 4 days, and then (b) effecting the transfer and apportioning of the T cells from the small scale culture into and amongst 2, 3 or 4 second containers that are larger in size than the first container, e.g., G-REX 500MCS containers, wherein in each second container the portion of the T cells from the small scale culture transferred to such second container is cultured in a larger scale culture for a period of about 5 days.

In some embodiments, the rapid second expansion is performed after the activation of T cells effected by the priming first expansion begins to decrease, abate, decay or subside.

In some embodiments, the rapid second expansion is performed after the activation of T cells effected by the priming first expansion has decreased by at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

In some embodiments, the rapid second expansion is performed after the activation of T cells effected by the priming first expansion has decreased by a percentage in the range of at or about 1% to 100%.

In some embodiments, the rapid second expansion is performed after the activation of T cells effected by the priming first expansion has decreased by a percentage in the range of at or about 1% to 10%, 10% to 20%, 20% to 30%, 30% to 40%, 40% to 50%, 50% to 60%, 60% to 70%, 70% to 80%, 80% to 90%, or 90% to 100%.

In some embodiments, the rapid second expansion is performed after the activation of T cells effected by the priming first expansion has decreased by at least at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In some embodiments, the rapid second expansion is performed after the activation of T cells effected by the priming first expansion has decreased by up to at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In some embodiments, the decrease in the activation of T cells effected by the priming first expansion is determined by a reduction in the amount of interferon gamma released by the T cells in response to stimulation with antigen.

In some embodiments, the priming first expansion of T cells is performed during a period of up to at or about 7 days or about 8 days.

In some embodiments, the priming first expansion of T cells is performed during a period of up to at or about 1 day, 2 days, 3 days, 4 days, 5 days, 6 days, 7 days, or 8 days.

In some embodiments, the priming first expansion of T cells is performed during a period of 1 day, 2 days, 3 days, 4 days, 5 days, 6 days, 7 days, or 8 days.

In some embodiments, the rapid second expansion of T cells is performed during a period of up to at or about 11 days.

In some embodiments, the rapid second expansion of T cells is performed during a period of up to at or about 1 day, 2 days, 3 days, 4 days, 5 days, 6 days, 7 days, 8 days, 9 days, 10 days or 11 days.

In some embodiments, the rapid second expansion of T cells is performed during a period of 1 day, 2 days, 3 days, 4 days, 5 days, 6 days, 7 days, 8 days, 9 days, 10 days or 11 days.

In some embodiments, the priming first expansion of T cells is performed during a period of from at or about 1 day to at or about 7 days and the rapid second expansion of T cells is performed during a period of from at or about 1 day to at or about 11 days.

In some embodiments, the priming first expansion of T cells is performed during a period of up to at or about 1 day, 2 days, 3 days, 4 days, 5 days, 6 days, 7 days, or 8 days and the rapid second expansion of T cells is performed during a period of up to at or about 1 day, 2 days, 3 days, 4 days, 5 days, 6 days, 7 days, 8 days, 9 days, 10 days or 11 days.

In some embodiments, the priming first expansion of T cells is performed during a period of from at or about 1 day to at or about 8 days and the rapid second expansion of T cells is performed during a period of from at or about 1 day to at or about 9 days.

In some embodiments, the priming first expansion of T cells is performed during a period of 8 days and the rapid second expansion of T cells is performed during a period of 9 days.

In some embodiments, the priming first expansion of T cells is performed during a period of from at or about 1 day to at or about 7 days and the rapid second expansion of T cells is performed during a period of from at or about 1 day to at or about 9 days.

In some embodiments, the priming first expansion of T cells is performed during a period of 7 days and the rapid second expansion of T cells is performed during a period of 9 days.

In some embodiments, the T cells are tumor infiltrating lymphocytes (TILs).

In some embodiments, the T cells are marrow infiltrating lymphocytes (MILs).

In some embodiments, the T cells are peripheral blood lymphocytes (PBLs).

In some embodiments, the T cells are obtained from a donor suffering from a cancer.

In some embodiments, the T cells are TILs obtained from a tumor excised from a patient suffering from a cancer.

In some embodiments, the T cells are MILs obtained from bone marrow of a patient suffering from a hematologic malignancy.

In some embodiments, the T cells are PBLs obtained from peripheral blood mononuclear cells (PBMCs) from a donor. In some embodiments, the donor is suffering from a cancer. In some embodiments, the donor is suffering from a hematologic malignancy.

In certain aspects of the present disclosure, immune effector cells, e.g., T cells, can be obtained from a unit of blood collected from a subject using any number of techniques known to the skilled artisan, such as FICOLL separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. In one aspect, T cells are isolated from peripheral blood lymphocytes by lysing the red blood cells and depleting the monocytes, for example, by centrifugation through a PERCOLL gradient or by counterflow centrifugal elutriation.

In some embodiments, the T cells are PBLs separated from whole blood or apheresis product enriched for lymphocytes from a donor. In some embodiments, the donor is suffering from a cancer. In some embodiments, the donor is suffering from a cancer. In some embodiments, the cancer is the cancer is selected from the group consisting of melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the donor is suffering from a tumor. In some embodiments, the tumor is a liquid tumor. In some embodiments, the tumor is a solid tumor. In some embodiments, the donor is suffering from a hematologic malignancy.

In certain aspects of the present disclosure, immune effector cells, e.g., T cells, can be obtained from a unit of blood collected from a subject using any number of techniques known to the skilled artisan, such as FICOLL separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. In one aspect, T cells are isolated from peripheral blood lymphocytes by lysing the red blood cells and depleting the monocytes, for example, by centrifugation through a PERCOLL gradient or by counterflow centrifugal elutriation.

In some embodiments, the T cells are PBLs separated from whole blood or apheresis product enriched for lymphocytes from a donor. In some embodiments, the donor is suffering from a cancer. In some embodiments, the cancer is the cancer is selected from the group consisting of melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the donor is suffering from a tumor. In some embodiments, the tumor is a liquid tumor. In some embodiments, the tumor is a solid tumor. In some embodiments, the donor is suffering from a hematologic malignancy. In some embodiments, the PBLs are isolated from whole blood or apheresis product enriched for lymphocytes by using positive or negative selection methods, i.e., removing the PBLs using a marker(s), e.g., CD3+CD45+, for T cell phenotype; or removing non-T cell phenotype cells, leaving PBLs. In other embodiments, the PBLs are isolated by gradient centrifugation. Upon isolation of PBLs from donor tissue, the priming first expansion of PBLs can be initiated by seeding a suitable number of isolated PBLs (in some embodiments, approximately 1×10⁷ PBLs) in the priming first expansion culture according to the priming first expansion step of any of the methods described herein.

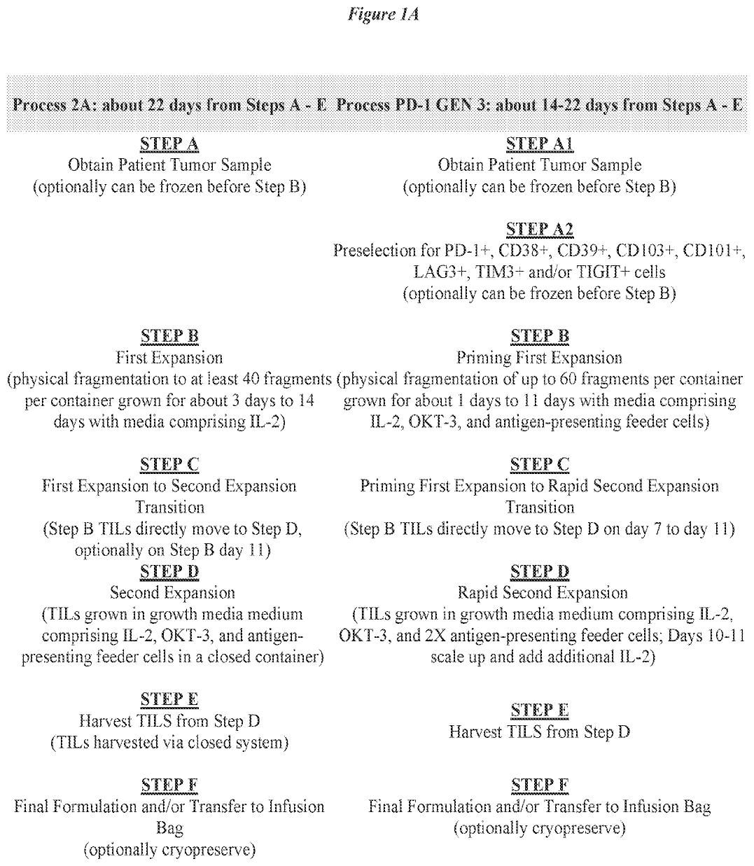

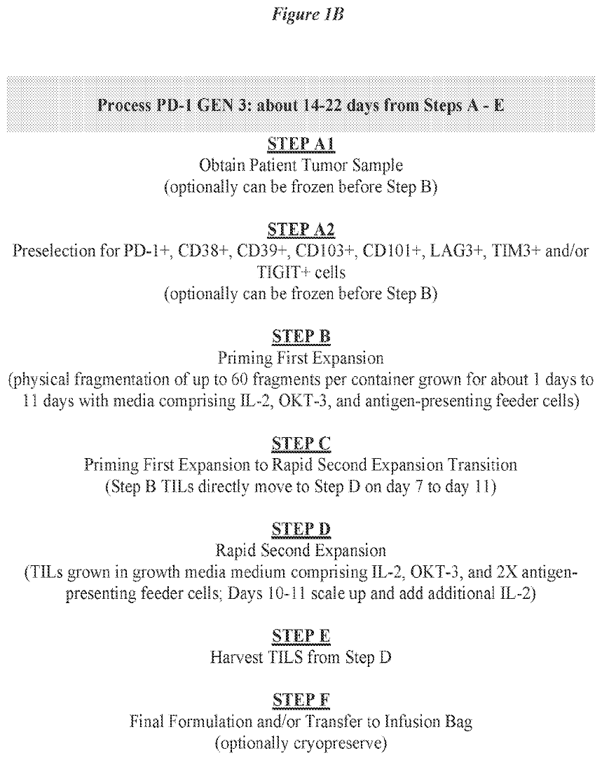

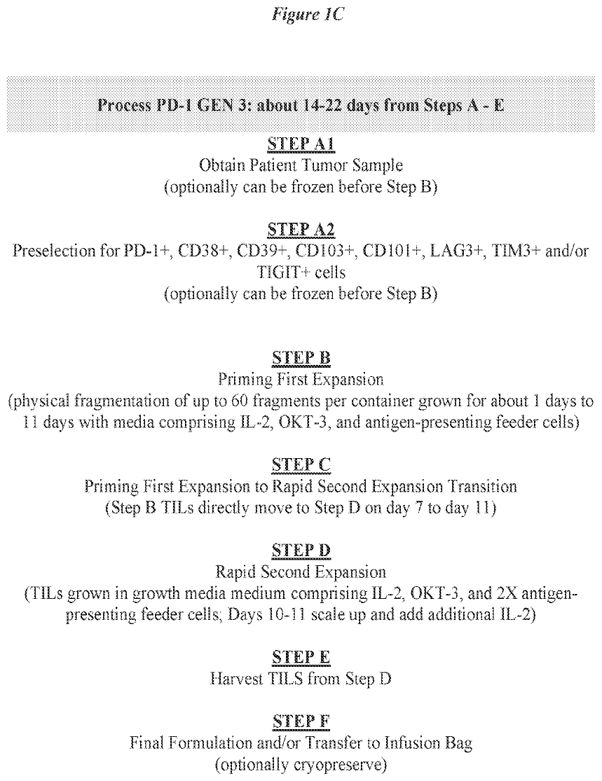

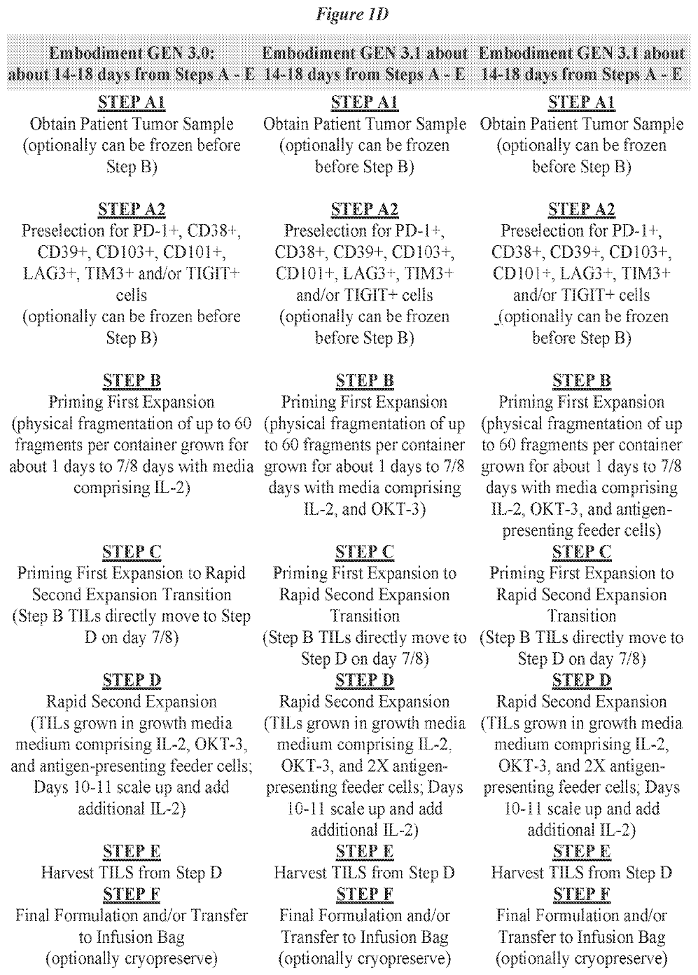

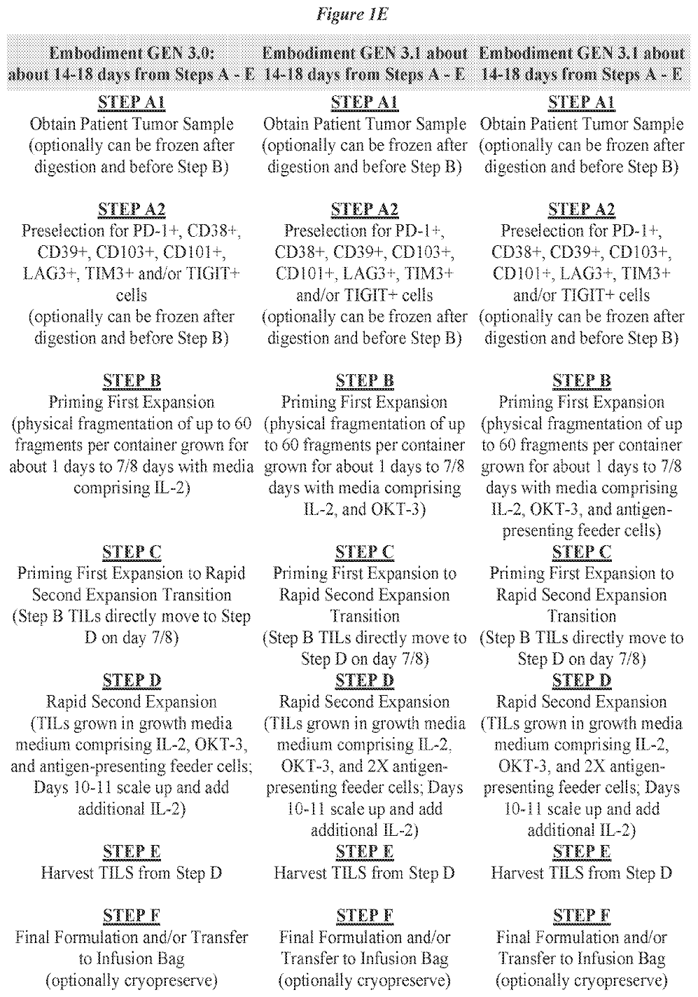

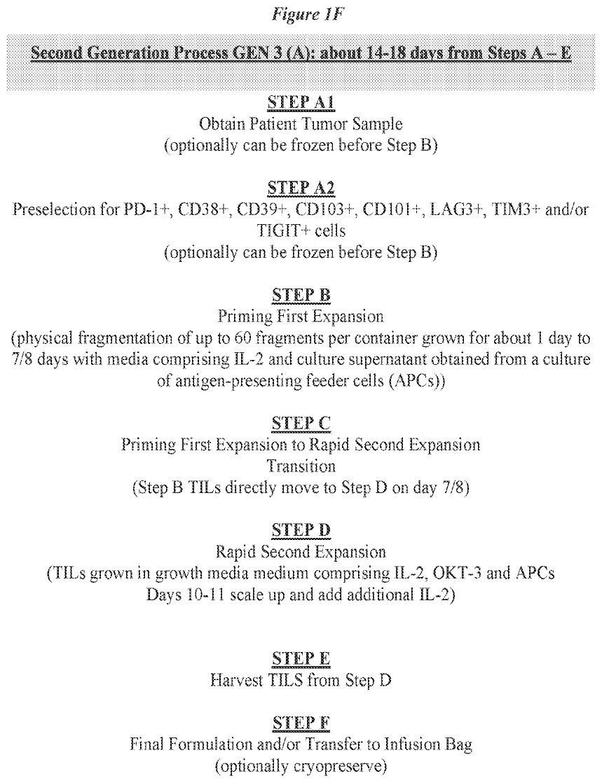

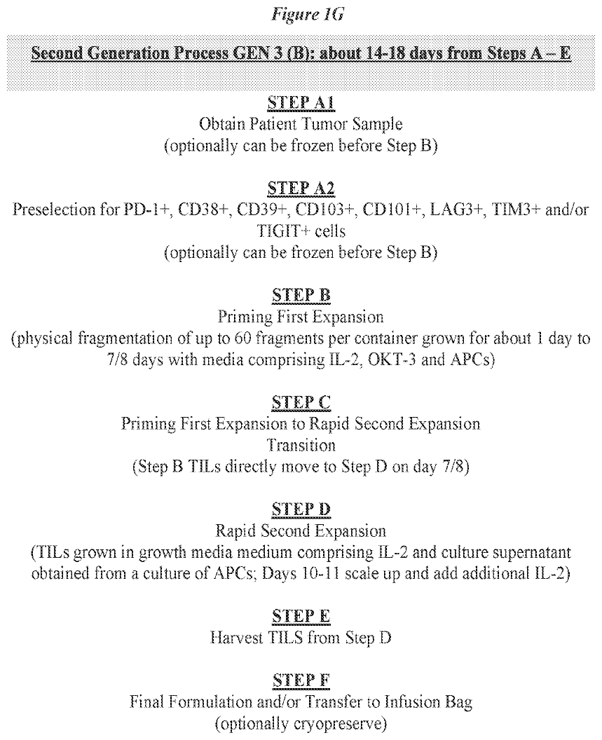

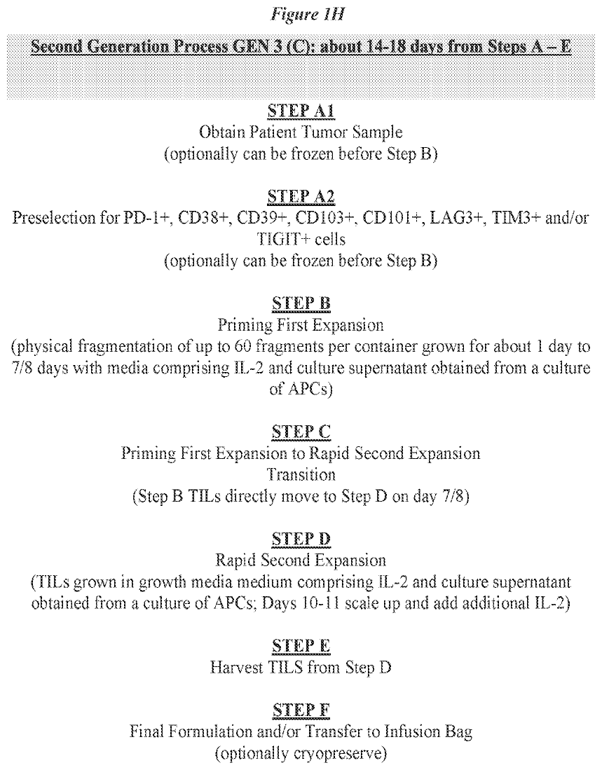

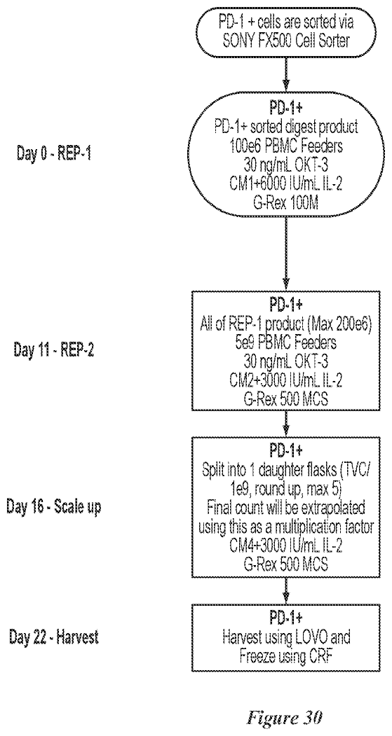

An exemplary TIL process known as process 3 (also referred to herein as GEN3) containing some of these features is depicted in FIG. 1 (in particular, e.g., FIG. 1B), and some of the advantages of this embodiment of the present invention over process 2A are described in FIGS. 1, 2, 30, and 31 (in particular, e.g., FIG. 1B). Two embodiments of process 3 are shown in FIGS. 1 and 30 (in particular, e.g., FIG. 1B). Process 2A or Gen 2 is also described in U.S. Patent Publication No. 2018/0280436, incorporated by reference herein in its entirety. The Gen 3 process is also described in USSN 62/755,954 filed on Nov. 5, 2018 (116983-5045-PR).

As discussed and generally outlined herein, TILs are taken from a patient sample and manipulated to expand their number prior to transplant into a patient using the TIL expansion process described herein and referred to as Gen 3. In some embodiments, the TILs may be optionally genetically manipulated as discussed below. In some embodiments, the TILs may be cryopreserved prior to or after expansion. Once thawed, they may also be restimulated to increase their metabolism prior to infusion into a patient.

In some embodiments, the priming first expansion (including processes referred herein as the pre-Rapid Expansion (Pre-REP), as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step B) is shortened to 1 to 8 days and the rapid second expansion (including processes referred to herein as Rapid Expansion Protocol (REP) as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step D) is shortened to 1 to 9 days, as discussed in detail below as well as in the examples and figures. In some embodiments, the priming first expansion (including processes referred herein as the pre-Rapid Expansion (Pre-REP), as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step B) is shortened to 1 to 8 days and the rapid second expansion (including processes referred to herein as Rapid Expansion Protocol (REP) as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step D) is shortened to 1 to 8 days, as discussed in detail below as well as in the examples and figures. In some embodiments, the priming first expansion (including processes referred herein as the pre-Rapid Expansion (Pre-REP), as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step B) is shortened to 1 to 7 days and the rapid second expansion (including processes referred to herein as Rapid Expansion Protocol (REP) as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step D) is shortened to 1 to 9 days, as discussed in detail below as well as in the examples and figures. In some embodiments, the priming first expansion (including processes referred herein as the pre-Rapid Expansion (Pre-REP), as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step B) is 1 to 7 days and the rapid second expansion (including processes referred to herein as Rapid Expansion Protocol (REP) as well as processes shown in FIG. 1 (in particular, e.g., FIG. 1B and/or FIG. 1C and/or FIG. 1D and/or FIG. 1E and/or FIG. 1F and/or FIG. 1G and/or FIG. 1H) as Step D) is 1 to 10 days, as discussed in detail below as well as in the examples and figures. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is shortened to 8 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 to 9 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is 8 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 8 to 9 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is shortened to 7 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 to 8 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is shortened to 8 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 8 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is 8 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 9 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is 8 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 10 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 to 10 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 8 to 10 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 9 to 10 days. In some embodiments, the priming first expansion (for example, an expansion described as Step B in FIG. 1 (in particular, e.g., FIG. 1B and/or FIG. 1C and/or FIG. 1D and/or FIG. 1E and/or FIG. 1F and/or FIG. 1G and/or FIG. 1H)) is shortened to 7 days and the rapid second expansion (for example, an expansion as described in Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 7 to 9 days. In some embodiments, the combination of the priming first expansion and rapid second expansion (for example, expansions described as Step B and Step D in FIG. 1 (in particular, e.g., FIG. 1B and/or FIG. 1C and/or FIG. 1D and/or FIG. 1E and/or FIG. 1F and/or FIG. 1G and/or FIG. 1H)) is 14-16 days, as discussed in detail below and in the examples and figures. Particularly, it is considered that certain embodiments of the present invention comprise a priming first expansion step in which TILs are activated by exposure to an anti-CD3 antibody, e.g., OKT-3 in the presence of IL-2 or exposure to an antigen in the presence of at least IL-2 and an anti-CD3 antibody e.g. OKT-3. In certain embodiments, the TILs which are activated in the priming first expansion step as described above are a first population of TILs i.e., which are a primary cell population.

The “Step” Designations A, B, C, etc., below are in reference to the non-limiting example in FIG. 1 (in particular, e.g., FIG. 1B and/or FIG. 1C and/or FIG. 1D and/or FIG. 1E and/or FIG. 1F and/or FIG. 1G and/or FIG. 1H) and in reference to certain non-limiting embodiments described herein. The ordering of the Steps below and in FIG. 1 (in particular, e.g., FIG. 1B and/or FIG. 1C and/or FIG. 1D and/or FIG. 1E and/or FIG. 1F and/or FIG. 1G and/or FIG. 1H) is exemplary and any combination or order of steps, as well as additional steps, repetition of steps, and/or omission of steps is contemplated by the present application and the methods disclosed herein.

A. STEP A: Obtain Patient Tumor Sample

In general, TILs are initially obtained from a patient tumor sample (“primary TILs”) or from circulating lymphocytes, such as peripherial blood lymphocytes, including perpherial blood lymphocytes having TIL-like characteristics, and are then expanded into a larger population for further manipulation as described herein, optionally cryopreserved, and optionally evaluated for phenotype and metabolic parameters as an indication of TIL health.

A patient tumor sample may be obtained using methods known in the art, generally via surgical resection, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). In some embodiments, the cancer is selected from cervical cancer, head and neck cancer (including, for example, head and neck squamous cell carcinoma (HNSCC), glioblastoma (GBM), gastrointestinal cancer, ovarian cancer, sarcoma, pancreatic cancer, bladder cancer, breast cancer, triple negative breast cancer, and non-small cell lung carcinoma. In some embodiments, useful TILs are obtained from malignant melanoma tumors, as these have been reported to have particularly high levels of TILs.

Once obtained, the tumor sample is generally fragmented using sharp dissection into small pieces of between 1 to about 8 mm³, with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

Tumor dissociating enzyme mixtures can include one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof.

In some embodiments, the dissociating enzymes are reconstituted from lyophilized enzymes. In some embodiments, lyophilized enzymes are reconstituted in an amount of sterile buffer such as HBSS.

In some instances, collagenase (such as animal free-type 1 collagenase) is reconstituted in 10 ml of sterile HBSS or another buffer. The lyophilized stock enzyme may be at a concentration of 2892 PZ U/vial. In some embodiments, collagenase is reconstituted in 5 ml to 15 ml buffer. In some embodiment, after reconstitution the collagenase stock ranges from about 100 PZ U/ml-about 400 PZ U/ml, e.g., about 100 PZ U/ml-about 400 PZ U/ml, about 100 PZ U/ml-about 350 PZ U/ml, about 100 PZ U/ml-about 300 PZ U/ml, about 150 PZ U/ml-about 400 PZ U/ml, about 100 PZ U/ml, about 150 PZ U/ml, about 200 PZ U/ml, about 210 PZ U/ml, about 220 PZ U/ml, about 230 PZ U/ml, about 240 PZ U/ml, about 250 PZ U/ml, about 260 PZ U/ml, about 270 PZ U/ml, about 280 PZ U/ml, about 289.2 PZ U/ml, about 300 PZ U/ml, about 350 PZ U/ml, or about 400 PZ U/ml.

In some embodiments, neutral protease is reconstituted in 1-ml of sterile HBSS or another buffer. The lyophilized stock enzyme may be at a concentration of 175 DMC U/vial. The lyophilized stock enzyme may be at a concentration of 175 DMC/mL. In some embodiments, after reconstitution the neutral protease stock ranges from about 100 DMC/ml-about 400 DMC/ml, e.g., about 100 DMC/ml-about 400 DMC/ml, about 100 DMC/ml-about 350 DMC/ml, about 100 DMC/ml-about 300 DMC/ml, about 150 DMC/ml-about 400 DMC/ml, about 100 DMC/ml, about 110 DMC/ml, about 120 DMC/ml, about 130 DMC/ml, about 140 DMC/ml, about 150 DMC/ml, about 160 DMC/ml, about 170 DMC/ml, about 175 DMC/ml, about 180 DMC/ml, about 190 DMC/ml, about 200 DMC/ml, about 250 DMC/ml, about 300 DMC/ml, about 350 DMC/ml, or about 400 DMC/ml.

In some embodiments, DNAse I is reconstituted in 1-ml of sterile HBSS or another buffer. The lyophilized stock enzyme was at a concentration of 4 KU/vial. In some embodiments, after reconstitution the DNase I stock ranges from about 1 KU/ml-10 KU/ml, e.g., about 1 KU/ml, about 2 KU/ml, about 3 KU/ml, about 4 KU/ml, about 5 KU/ml, about 6 KU/nil, about 7 KU/ml, about 8 KU/ml, about 9 KU/ml, or about 10 KU/ml.

In some embodiments, the stock of enzymes could change so verify the concentration of the lyophilized stock and amend the final amount of enzyme added to the digest cocktail accordingly.

In some embodiments, the enzyme mixture includes neutral protease, DNase, and collagenase.

In some embodiments, the enzyme mixture includes about 10.2-ul of neutral protease (0.36 DMC U/ml), 21.3-ul of collagenase (1.2 PZ/ml) and 250-ul of DNAse I (200 U/ml) in about 4.7-ml of sterile HBSS.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and hyaluronidase. In some embodiments, the tumors are digested in in an enzyme mixture comprising collagenase, DNase, and hyaluronidase for 1-2 hours. In some embodiments, the tumors are digested in in an enzyme mixture comprising collagenase, DNase, and hyaluronidase for 1-2 hours at 37° C., 5% CO₂. In some embodiments, the tumors are digested in in an enzyme mixture comprising collagenase, DNase, and hyaluronidase for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture. In some embodiments, the tumors are digested and then frozen prior to continuing on with the selection process. In some embodiments, the tumors are digested and then frozen prior to continuing on with the expansion process.

In some embodiments, the tumor is reconstituted with the lyophilized enzymes in a sterile buffer. In some embodiments, the buffer is sterile HBSS.

In some embodiments, the enxyme mixture comprises collagenase. In some embodiments, the collagenase is collagenase IV. In some embodiments, the working stock for the collagenase is a 100 mg/ml 10X working stock.

In some embodiments, the enzyme mixture comprises DNAse. In some embodiments, the working stock for the DNAse is a 10,000 IU/ml 10X working stock.

In some embodiments, the enzyme mixture comprises hyaluronidase. In some embodiments, the working stock for the hyaluronidase is a 10-mg/ml 10X working stock.

In some embodiments, the enzyme mixture comprises 10 mg/ml collagenase, 1000 IU/ml DNAse, and 1 mg/ml hyaluronidase.

In some embodiments, the enzyme mixture comprises 10 mg/ml collagenase, 500 IU/ml DNAse, and 1 mg/ml hyaluronidase.

In some embodiments, the enzyme mixture comprises about 10 mg/ml collagenase, about 1000 IU/ml DNAse, and about 1 mg/ml hyaluronidase.

In general, the cell suspension obtained from the tumor is called a “primary cell population” or a “freshly obtained” or a “freshly isolated” cell population. In certain embodiments, the freshly obtained cell population of TILs is exposed to a cell culture medium comprising antigen presenting cells, IL-12 and OKT-3.

In some embodiments, the digest can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

In some embodiments, fragmentation includes physical fragmentation, including for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients. In some embodiments, TILs can be initially cultured from enzymatic tumor digests and tumor fragments obtained from patients.

In some embodiments, where the tumor is a solid tumor, the tumor undergoes physical fragmentation after the tumor sample is obtained in, for example, Step A (as provided in FIG. 1 (in particular, e.g., FIG. 1B and/or FIG. 1C and/or FIG. 1D and/or FIG. 1E and/or FIG. 1F and/or FIG. 1G and/or FIG. 1H)). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the step of fragmentation is an in vitro or ex-vivo process. In some embodiments, the tumor is fragmented and 10, 20, 30, 40 or more fragments or pieces are placed in each container for the priming first expansion. In some embodiments, the tumor is fragmented and 30 or 40 fragments or pieces are placed in each container for the priming first expansion. In some embodiments, the tumor is fragmented and 40 fragments or pieces are placed in each container for the priming first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumor fragments are 1-4 mmx 1-4 mm x 1-4 mm. In some embodiments, the tumor fragments are 1 mmx 1 mm×1 mm. In some embodiments, the tumor fragments are 2 mmx 2 mm×2 mm. In some embodiments, the tumor fragments are 3 mmx 3 mm×3 mm. In some embodiments, the tumor fragments are 4 mm×4 mm×4 mm.

In some embodiments, the tumors are fragmented in order to minimize the amount of hemorrhagic, necrotic, and/or fatty tissues on each piece. In some embodiments, the tumors are fragmented in order to minimize the amount of hemorrhagic tissue on each piece. In some embodiments, the tumors are fragmented in order to minimize the amount of necrotic tissue on each piece. In some embodiments, the tumors are fragmented in order to minimize the amount of fatty tissue on each piece. In certain embodiments, the step of fragmentation of the tumor is an in vitro or ex-vivo method.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, Calif.). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, the cell suspension prior to the priming first expansion step is called a “primary cell population” or a “freshly obtained” or “freshly isolated” cell population.

In some embodiments, cells can be optionally frozen after sample isolation (e.g., after obtaining the tumor sample and/or after obtaining the cell suspension from the tumor sample) and stored frozen prior to entry into the expansion described in Step B, which is described in further detail below, as well as exemplified in FIG. 1 (in particular, e.g., FIG. 1B and/or FIG. 1C and/or FIG. 1D and/or FIG. 1E and/or FIG. 1F and/or FIG. 1G and/or FIG. 1H).

1. Core/Small Biopsy Derived TILs

In some embodiments, TILs are initially obtained from a patient tumor sample (“primary TILs”) obtained by a core biopsy or similar procedure and then expanded into a larger population for further manipulation as described herein, optionally cryopreserved, and optionally evaluated for phenotype and metabolic parameters.

In some embodiments, a patient tumor sample may be obtained using methods known in the art, generally via small biopsy, core biopsy, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. In some embodiments, the sample can be from multiple small tumor samples or biopsies. In some embodiments, the sample can comprise multiple tumor samples from a single tumor from the same patient. In some embodiments, the sample can comprise multiple tumor samples from one, two, three, or four tumors from the same patient. In some embodiments, the sample can comprise multiple tumor samples from multiple tumors from the same patient. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). In some embodiments, the cancer is selected from cervical cancer, head and neck cancer (including, for example, head and neck squamous cell carcinoma (HNSCC)), glioblastoma (GBM), gastrointestinal cancer, ovarian cancer, sarcoma, pancreatic cancer, bladder cancer, breast cancer, triple negative breast cancer, and non-small cell lung carcinoma (NSCLC). In some embodiments, useful TILs are obtained from malignant melanoma tumors, as these have been reported to have particularly high levels of TILs.

In general, the cell suspension obtained from the tumor core or fragment is called a “primary cell population” or a “freshly obtained” or a “freshly isolated” cell population. In certain embodiments, the freshly obtained cell population of TILs is exposed to a cell culture medium comprising antigen presenting cells, IL-2 and OKT-3.

In some embodiments, if the tumor is metastatic and the primary lesion has been efficiently treated/removed in the past, removal of one of the metastatic lesions may be needed. In some embodiments, the least invasive approach is to remove a skin lesion, or a lymph node on the neck or axillary area when available. In some embodiments, a skin lesion is removed or small biopsy thereof is removed. In some embodiments, a lymph node or small biopsy thereof is removed. In some embodiments, a lung or liver metastatic lesion, or an intra-abdominal or thoracic lymph node or small biopsy can thereof can be employed.

In some embodiments, the tumor is a melanoma. In some embodiments, the small biopsy for a melanoma comprises a mole or portion thereof.

In some embodiments, the small biopsy is a punch biopsy. In some embodiments, the punch biopsy is obtained with a circular blade pressed into the skin. In some embodiments, the punch biopsy is obtained with a circular blade pressed into the skin around a suspicious mole. In some embodiments, the punch biopsy is obtained with a circular blade pressed into the skin, and a round piece of skin is removed. In some embodiments, the small biopsy is a punch biopsy and round portion of the tumor is removed.

In some embodiments, the small biopsy is an excisional biopsy. In some embodiments, the small biopsy is an excisional biopsy and the entire mole or growth is removed. In some embodiments, the small biopsy is an excisional biopsy and the entire mole or growth is removed along with a small border of normal-appearing skin.

In some embodiments, the small biopsy is an incisional biopsy. In some embodiments, the small biopsy is an incisional biopsy and only the most irregular part of a mole or growth is taken. In some embodiments, the small biopsy is an incisional biopsy and the incisional biopsy is used when other techniques can't be completed, such as if a suspicious mole is very large.

In some embodiments, the small biopsy is a lung biopsy. In some embodiments, the small biopsy is obtained by bronchoscopy. Generally, bronchoscopy, the patient is put under anesthesia, and a small tool goes through the nose or mouth, down the throat, and into the bronchial passages, where small tools are used to remove some tissue. In some embodiments, where the tumor or growth cannot be reached via bronchoscopy, a transthoracic needle biopsy can be employed. Generally, for a transthoracic needle biopsy, the patient is also under anesthesia and a needle is inserted through the skin directly into the suspicious spot to remove a small sample of tissue. In some embodiments, a transthoracic needle biopsy may require interventional radiology (for example, the use of x-rays or CT scan to guide the needle). In some embodiments, the small biopsy is obtained by needle biopsy. In some embodiments, the small biopsy is obtained endoscopic ultrasound (for example, an endoscope with a light and is placed through the mouth into the esophagus). In some embodiments, the small biopsy is obtained surgically.

In some embodiments, the small biopsy is a head and neck biopsy. In some embodiments; the small biopsy is an incisional biopsy. In some embodiments, the small biopsy is an incisional biopsy, wherein a small piece of tissue is cut from an abnormal-looking area. In some embodiments, if the abnormal region is easily accessed, the sample may be taken without hospitalization. In some embodiments, if the tumor is deeper inside the mouth or throat, the biopsy may need to be done in an operating room, with general anesthesia. In some embodiments, the small biopsy is an excisional biopsy. In some embodiments, the small biopsy is an excisional biopsy, wherein the whole area is removed. In some embodiments, the small biopsy is a fine needle aspiration (FNA). In some embodiments, the small biopsy is a fine needle aspiration (FNA), wherein a very thin needle attached to a syringe is used to extract (aspirate) cells from a tumor or lump. In some embodiments, the small biopsy is a punch biopsy. In some embodiments, the small biopsy is a punch biopsy, wherein punch forceps are used to remove a piece of the suspicious area.

In some embodiments, the small biopsy is a cervical biopsy. In some embodiments, the small biopsy is obtained via colposcopy. Generally, colposcopy methods employ the use of a lighted magnifying instrument attached to magnifying binoculars (a colposcope) which is then used to biopsy a small section of the surface of the cervix. In some embodiments, the small biopsy is a conization/cone biopsy. In some embodiments, the small biopsy is a conization/cone biopsy, wherein an outpatient surgery may be needed to remove a larger piece of tissue from the cervix. In some embodiments, the cone biopsy, in addition to helping to confirm a diagnosis, a cone biopsy can serve as an initial treatment.

The term “solid tumor” refers to an abnormal mass of tissue that usually does not contain cysts or liquid areas. Solid tumors may be benign or malignant. The term “solid tumor cancer refers to malignant, neoplastic, or cancerous solid tumors. Solid tumor cancers include, but are not limited to, sarcomas, carcinomas, and lymphomas, such as cancers of the lung, breast, triple negative breast cancer, prostate, colon, rectum, and bladder. In some embodiments, the cancer is selected from cervical cancer, head and neck cancer, glioblastoma, ovarian cancer, sarcoma, pancreatic cancer, bladder cancer, breast cancer, triple negative breast cancer, and non-small cell lung carcinoma. The tissue structure of solid tumors includes interdependent tissue compartments including the parenchyma (cancer cells) and the supporting stromal cells in which the cancer cells are dispersed and which may provide a supporting microenvironment.

In some embodiments, the sample from the tumor is obtained as a fine needle aspirate (FNA), a core biopsy, a small biopsy (including, for example, a punch biopsy). In some embodiments, sample is placed first into a G-Rex 10. In some embodiments, sample is placed first into a G-Rex 10 when there are 1 or 2 core biopsy and/or small biopsy samples. In some embodiments, sample is placed first into a G-Rex 100 when there are 3, 4, 5, 6, 8, 9, or 10 or more core biopsy and/or small biopsy samples. In some embodiments, sample is placed first into a G-Rex 500 when there are 3, 4, 5, 6, 8, 9, or 10 or more core biopsy and/or small biopsy samples.

The FNA can be obtained from a tumor selected from the group consisting of lung, melanoma, head and neck, cervical, ovarian, pancreatic, glioblastoma, colorectal, and sarcoma. In some embodiments, the FNA is obtained from a lung tumor, such as a lung tumor from a patient with non-small cell lung cancer (NSCLC). In some cases, the patient with NSCLC has previously undergone a surgical treatment.

TILs described herein can be obtained from an FNA sample. In some cases, the FNA sample is obtained or isolated from the patient using a fine gauge needle ranging from an 18 gauge needle to a 25 gauge needle. The fine gauge needle can be 18 gauge, 19 gauge, 20 gauge, 21 gauge, 22 gauge, 23 gauge, 24 gauge, or 25 gauge. In some embodiments, the FNA sample from the patient can contain at least 400,000 TILs, e.g., 400,000 TILs, 450,000 TILs, 500,000 TILs, 550,000 TILs, 600,000 TILs, 650,000 TILs, 700,000 TILs, 750,000 TILs, 800,000 TILs, 850,000 TILs, 900,000 TILs, 950,000 TILs, or more.

In some cases, the TILs described herein are obtained from a core biopsy sample. In some cases, the core biopsy sample is obtained or isolated from the patient using a surgical or medical needle ranging from an 11 gauge needle to a 16 gauge needle. The needle can be 11 gauge, 12 gauge, 13 gauge, 14 gauge, 15 gauge, or 16 gauge. In some embodiments, the core biopsy sample from the patient can contain at least 400,000 TILs, e.g., 400,000 TILs, 450,000 TILs, 500,000 TILs, 550,000 TILs, 600,000 TILs, 650,000 TILs, 700,000 TILs, 750,000 TILs, 800,000 TILs, 850,000 TILs, 900,000 TILs, 950,000 TILs, or more.

In general, the harvested cell suspension is called a “primary cell population” or a “freshly harvested” cell population.

In some embodiments, the TILs are not obtained from tumor digests. In some embodiments, the solid tumor cores are not fragmented.

In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, Calif.). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, obtaining the first population of TILs comprises a multilesional sampling method.

Tumor dissociating enzyme mixtures can include one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof.

In some embodiments, the dissociating enzymes are reconstituted from lyophilized enzymes. In some embodiments, lyophilized enzymes are reconstituted in an amount of sterile buffer such as HBSS.

In some instances, collagenase (such as animal free-type 1 collagenase) is reconstituted in 10 ml of sterile HBSS or another buffer. The lyophilized stock enzyme may be at a concentration of 2892 PZ U/vial. In some embodiments, collagenase is reconstituted in 5 ml to 15 ml buffer. In some embodiment, after reconstitution the collagenase stock ranges from about 100 PZ U/ml-about 400 PZ U/ml, e.g., about 100 PZ U/ml-about 400 PZ U/ml, about 100 PZ U/ml-about 350 PZ U/ml, about 100 PZ U/ml-about 300 PZ U/ml, about 150 PZ U/ml-about 400 PZ U/ml, about 100 PZ U/ml, about 150 PZ U/ml, about 200 PZ U/ml, about 210 PZ U/ml, about 220 PZ U/ml, about 230 PZ U/ml, about 240 PZ U/ml, about 250 PZ U/ml, about 260 PZ U/ml, about 270 PZ U/ml, about 280 PZ U/ml, about 289.2 PZ U/ml, about 300 PZ U/ml, about 350 PZ U/ml, or about 400 PZ U/ml.

In some embodiments neutral protease is reconstituted in 1-ml of sterile HBSS or another buffer. The lyophilized stock enzyme may be at a concentration of 175 DMC U/vial. The lyophilized stock enzyme may be at a concentration of 175 DMC/mL. In some embodiments, after reconstitution the neutral protease stock ranges from about 100 DMC/ml-about 400 DMC/ml, e.g., about 100 DMC/ml-about 400 DMC/ml, about 100 DMC/ml-about 350 DMC/ml, about 100 DMC/ml-about 300 DMC/ml, about 150 DMC/ml-about 400 DMC/ml, about 100 DMC/ml, about 110 DMC/ml, about 120 DMC/ml, about 130 DMC/ml, about 140 DMC/ml, about 150 DMC/ml, about 160 DMC/ml, about 170 DMC/ml, about 175 DMC/ml, about 180 DMC/ml, about 190 DMC/ml, about 200 DMC/ml, about 250 DMC/ml, about 300 DMC/ml, about 350 DMC/ml, or about 400 DMC/ml.

In some embodiments, DNAse I is reconstituted in 1-ml of sterile HBSS or another buffer. The lyophilized stock enzyme was at a concentration of 4 KU/vial. In some embodiments, after reconstitution the DNase I stock ranges from about 1 KU/ml-10 KU/ml, e.g., about 1 KU/ml, about 2 KU/ml, about 3 KU/ml, about 4 KU/ml, about 5 KU/ml, about 6 KU/ml, about 7 KU/ml, about 8 KU/ml, about 9 KU/ml, or about 10 KU/ml.

In some embodiments, the stock of enzymes could change so verify the concentration of the lyophilized stock and amend the final amount of enzyme added to the digest cocktail accordingly.

In some embodiments, the enzyme mixture includes neutral protease, collagenase and DNase

In some embodiments, the enzyme mixture includes about 10.2-ul of neutral protease (0.36 DMC U/ml), 21.3-ul of collagenase (1.2 PZ/ml) and 250-ul of DNAse I (200 U/ml) in about 4.7-ml of sterile HBSS.

2. Pleural Effusion TILs

In some embodiments, the sample is a pleural fluid sample. In some embodiments, the source of the TILs for expansion according to the processes described herein is a pleural fluid sample. In some embodiments, the sample is a pleural effusion derived sample. In some embodiments, the source of the TILs for expansion according to the processes described herein is a pleural effusion derived sample. See, for example, methods described in U.S. Patent Publication US 2014/0295426, incorporated herein by reference in its entirety for all purposes.

In some embodiments, any pleural fluid or pleural effusion suspected of and/or containing TILs can be employed. Such a sample may be derived from a primary or metastatic lung cancer, such as NSCLC or SCLC. In some embodiments, the sample may be secondary metastatic cancer cells which originated from another organ, e.g., breast, ovary, colon or prostate. In some embodiments, the sample for use in the expansion methods described herein is a pleural exudate. In some embodiments, the sample for use in the expansion methods described herein is a pleural transudate. Other biological samples may include other serous fluids containing TILs, including, e.g., ascites fluid from the abdomen or pancreatic cyst fluid. Ascites fluid and pleural fluids involve very similar chemical systems: both the abdomen and lung have mesothelial lines and fluid forms in the pleural space and abdominal spaces in the same matter in malignancies and such fluids in some embodiments contain TILs. In some embodiments, wherein the disclosure exemplifies pleural fluid, the same methods may be performed with similar results using ascites or other cyst fluids containing TILs.

In some embodiments, the pleural fluid is in unprocessed form, directly as removed from the patient. In some embodiments, the unprocessed pleural fluid is placed in a standard blood collection tube, such as an EDTA or Heparin tube, prior to the contacting step. In some embodiments, the unprocessed pleural fluid is placed in a standard CellSave® tube (Veridex) prior to the contacting step. In some embodiments, the sample is placed in the CellSave tube immediately after collection from the patient to avoid a decrease in the number of viable TILs. The number of viable TILs can decrease to a significant extent within 24 hours, if left in the untreated pleural fluid, even at 4° C. In some embodiments, the sample is placed in the appropriate collection tube within 1 hour, 5 hours, 10 hours, 15 hours, or up to 24 hours after removal from the patient. In some embodiments, the sample is placed in the appropriate collection tube within 1 hour, 5 hours, 10 hours, 15 hours, or up to 24 hours after removal from the patient at 4° C.

In some embodiments, the pleural fluid sample from the chosen subject may be diluted. In some embodiments, the dilution is 1:10 pleural fluid to diluent. In other embodiments, the dilution is 1:9 pleural fluid to diluent. In other embodiments, the dilution is 1:8 pleural fluid to diluent. In other embodiments, the dilution is 1:5 pleural fluid to diluent. In other embodiments, the dilution is 1:2 pleural fluid to diluent. In other embodiments, the dilution is 1:1 pleural fluid to diluent. In some embodiments, diluents include saline, phosphate buffered saline, another buffer or a physiologically acceptable diluent. In some embodiments, the sample is placed in the CellSave tube immediately after collection from the patient and dilution to avoid a decrease in the viable TILs, which may occur to a significant extent within 24-48 hours, if left in the untreated pleural fluid, even at 4° C. In some embodiments, the pleural fluid sample is placed in the appropriate collection tube within 1 hour, 5 hours, 10 hours, 15 hours, 24 hours, 36 hours, up to 48 hours after removal from the patient, and dilution. In some embodiments, the pleural fluid sample is placed in the appropriate collection tube within 1 hour, 5 hours, 10 hours, 15 hours, 24 hours, 36 hours, up to 48 hours after removal from the patient, and dilution at 4° C.

In still another embodiment, pleural fluid samples are concentrated by conventional means prior further processing steps. In some embodiments, this pre-treatment of the pleural fluid is preferable in circumstances in which the pleural fluid must be cryopreserved for shipment to a laboratory performing the method or for later analysis (e.g., later than 24-48 hours post-collection). In some embodiments, the pleural fluid sample is prepared by centrifuging the pleural fluid sample after its withdrawal from the subject and resuspending the centrifugate or pellet in buffer. In some embodiments, the pleural fluid sample is subjected to multiple centrifugations and resuspensions, before it is cryopreserved for transport or later analysis and/or processing.

In some embodiments, pleural fluid samples are concentrated prior to further processing steps by using a filtration method. In some embodiments, the pleural fluid sample used in the contacting step is prepared by filtering the fluid through a filter containing a known and essentially uniform pore size that allows for passage of the pleural fluid through the membrane but retains the tumor cells. In some embodiments, the diameter of the pores in the membrane may be at least 4 μM. In other embodiments the pore diameter may be 5 μM or more, and in other embodiment, any of 6, 7, 8, 9, or 10 μM. After filtration, the cells, including TILs, retained by the membrane may be rinsed off the membrane into a suitable physiologically acceptable buffer. Cells, including TILs, concentrated in this way may then be used in the contacting step of the method.

In some embodiment, pleural fluid sample (including, for example, the untreated pleural fluid), diluted pleural fluid, or the resuspended cell pellet, is contacted with a lytic reagent that differentially lyses non-nucleated red blood cells present in the sample. In some embodiments, this step is performed prior to further processing steps in circumstances in which the pleural fluid contains substantial numbers of RBCs. Suitable lysing reagents include a single lytic reagent or a lytic reagent and a quench reagent, or a lytic agent, a quench reagent and a fixation reagent. Suitable lytic systems are marketed commercially and include the BD Pharm Lyse™ system (Becton Dickenson). Other lytic systems include the Versalyse™ system, the FACSlyse™ system (Becton Dickenson), the Immunoprep™ system or Erythrolyse II system (Beckman Coulter, Inc.), or an ammonium chloride system. In some embodiments, the lytic reagent can vary with the primary requirements being efficient lysis of the red blood cells, and the conservation of the TILs and phenotypic properties of the TILs in the pleural fluid. In addition to employing a single reagent for lysis, the lytic systems useful in methods described herein can include a second reagent, e.g., one that quenches or retards the effect of the lytic reagent during the remaining steps of the method, e.g., Stabilyse™ reagent (Beckman Coulter, Inc.). A conventional fixation reagent may also be employed depending upon the choice of lytic reagents or the preferred implementation of the method.

In some embodiments, the pleural fluid sample, unprocessed, diluted or multiply centrifuged or processed as described herein above is cryopreserved at a temperature of about −140° C. prior to being further processed and/or expanded as provided herein.

3. Methods of Expanding Peripheral Blood Lymphocytes (PBLs) from Peripheral Blood

PBL Method 1. In some embodiments of the invention, PBLs are expanded using the processes described herein. In some embodiments of the invention, the method comprises obtaining a PBMC sample from whole blood. In some embodiments, the method comprises enriching T-cells by isolating pure T-cells from PBMCs using negative selection of a non-CD19+ fraction. In some embodiments, the method comprises enriching T-cells by isolating pure T-cells from PBMCs using magnetic bead-based negative selection of a non-CD19+ fraction.

In some embodiments of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec).

PBL Method 2. In some embodiments of the invention, PBLs are expanded using PBL Method 2, which comprises obtaining a PBMC sample from whole blood. The T-cells from the PBMCs are enriched by incubating the PBMCs for at least three hours at 37° C. and then isolating the non-adherent cells.

In some embodiments of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted.

PBL Method 3. In some embodiments of the invention, PBLs are expanded using PBL Method 3, which comprises obtaining a PBMC sample from peripheral blood. B-cells are isolated using a CD19+ selection and T-cells are selected using negative selection of the non-CD19+ fraction of the PBMC sample.

In some embodiments of the invention, PBL Method 3 is performed as follows: On Day 0, cryopreserved PBMCs derived from peripheral blood are thawed and counted. CD19+ B-cells are sorted using a CD19 Multisort Kit, Human (Miltenyi Biotec). Of the non-CD19+ cell fraction, T-cells are purified using the Human Pan T-cell Isolation Kit and LS Columns (Miltenyi Biotec).

In some embodiments, PBMCs are isolated from a whole blood sample. In some embodiments, the PBMC sample is used as the starting material to expand the PBLs. In some embodiments, the sample is cryopreserved prior to the expansion process. In other embodiments, a fresh sample is used as the starting material to expand the PBLs. In some embodiments of the invention, T-cells are isolated from PBMCs using methods known in the art. In some embodiments, the T-cells are isolated using a Human Pan T-cell isolation kit and LS columns. In some embodiments of the invention, T-cells are isolated from PBMCs using antibody selection methods known in the art, for example, CD19 negative selection.

In some embodiments of the invention, the PBMC sample is incubated for a period of time at a desired temperature effective to identify the non-adherent cells. In some embodiments of the invention, the incubation time is about 3 hours. In some embodiments of the invention, the temperature is about 37° Celsius. The non-adherent cells are then expanded using the process described above.

In some embodiments, the PBMC sample is from a subject or patient who has been optionally pre-treated with a regimen comprising a kinase inhibitor or an ITK inhibitor. In some embodiments, the tumor sample is from a subject or patient who has been pre-treated with a regimen comprising a kinase inhibitor or an ITK inhibitor. In some embodiments, the PBMC sample is from a subject or patient who has been pre-treated with a regimen comprising a kinase inhibitor or an ITK inhibitor, has undergone treatment for at least 1 month, at least 2 months, at least 3 months, at least 4 months, at least 5 months, at least 6 months, or 1 year or more. In other embodiments, the PBMCs are derived from a patient who is currently on an ITK inhibitor regimen, such as ibrutinib.

In some embodiments, the PBMC sample is from a subject or patient who has been pre-treated with a regimen comprising a kinase inhibitor or an ITK inhibitor and is refractory to treatment with a kinase inhibitor or an ITK inhibitor, such as ibrutinib.

In some embodiments, the PBMC sample is from a subject or patient who has been pre-treated with a regimen comprising a kinase inhibitor or an ITK inhibitor but is no longer undergoing treatment with a kinase inhibitor or an ITK inhibitor. In some embodiments, the PBMC sample is from a subject or patient who has been pre-treated with a regimen comprising a kinase inhibitor or an ITK inhibitor but is no longer undergoing treatment with a kinase inhibitor or an ITK inhibitor and has not undergone treatment for at least 1 month, at least 2 months, at least 3 months, at least 4 months, at least 5 months, at least 6 months, or at least 1 year or more. In other embodiments, the PBMCs are derived from a patient who has prior exposure to an ITK inhibitor, but has not been treated in at least 3 months, at least 6 months, at least 9 months, or at least 1 year.

In some embodiments of the invention, at Day 0, cells are selected for CD19+ and sorted accordingly. In some embodiments of the invention, the selection is made using antibody binding beads. In some embodiments of the invention, pure T-cells are isolated on Day 0 from the PBMCs.

In some embodiments of the invention, for patients that are not pre-treated with ibrutinib or other ITK inhibitor, 10-15 ml of Buffy Coat will yield about 5×10⁹ PBMC, which, in turn, will yield about 5.5×10⁷ PBLs.

In some embodiments of the invention, for patients that are pre-treated with ibrutinib or other ITK inhibitor, the expansion process will yield about 20×10⁹ PBLs. In some embodiments of the invention, 40.3×10⁶ PBMCs will yield about 4.7×10⁵ PBLs.

In any of the foregoing embodiments, PBMCs may be derived from a whole blood sample, by apheresis, from the buffy coat, or from any other method known in the art for obtaining PBMCs.

4. Methods of Expanding Marrow Infiltrating Lymphocytes (MILs) from PBMCs Derived from Bone Marrow

MIL Method 3. In some embodiments of the invention, the method comprises obtaining PBMCs from the bone marrow. On Day 0, the PBMCs are selected for CD3+/CD33+/CD20+/CD14+ and sorted, and the non-CD3+/CD33+/CD20+/CD14+ cell fraction is sonicated and a portion of the sonicated cell fraction is added back to the selected cell fraction.

In some embodiments of the invention, MIL Method 3 is performed as follows: On Day 0, a cryopreserved sample of PBMCs is thawed and PBMCs are counted. The cells are stained with CD3, CD33, CD20, and CD14 antibodies and sorted using a S3e cell sorted (Bio-Rad). The cells are sorted into two fractions—an immune cell fraction (or the MIL fraction) (CD3+CD33+CD2O+CD14+) and an AML blast cell fraction (non-CD3+CD33+CD2O+CD14+).

In some embodiments of the invention, PBMCs are obtained from bone marrow. In some embodiments, the PBMCs are obtained from the bone marrow through apheresis, aspiration, needle biopsy, or other similar means known in the art. In some embodiments, the PBMCs are fresh. In other embodiments, the PBMCs are cryopreserved.

In some embodiments of the invention, MILs are expanded from 10-50 ml of bone marrow aspirate. In some embodiments of the invention, 10 ml of bone marrow aspirate is obtained from the patient. In other embodiments, 20 ml of bone marrow aspirate is obtained from the patient. In other embodiments, 30 ml of bone marrow aspirate is obtained from the patient. In other embodiments, 40 ml of bone marrow aspirate is obtained from the patient. In other embodiments, 50 ml of bone marrow aspirate is obtained from the patient.

In some embodiments of the invention, the number of PBMCs yielded from about 10-50 ml of bone marrow aspirate is about 5×10⁷ to about 10×10⁷ PBMCs. In other embodiments, the number of PMBCs yielded is about 7×10⁷ PBMCs.

In some embodiments of the invention, about 5×10⁷ to about 10×10⁷ PBMCs, yields about 0.5×10⁶ to about 1.5×10⁶ MILs. In some embodiments of the invention, about 1×10⁶ MILs is yielded.

In some embodiments of the invention, 12×10⁶ PBMC derived from bone marrow aspirate yields approximately 1.4×10⁵ MILs.

In any of the foregoing embodiments, PBMCs may be derived from a whole blood sample, from bone marrow, by apheresis, from the buffy coat, or from any other method known in the art for obtaining PBMCs.

5. PD-1—Preselection Selection for PD-1 (as exemplified in Step A2 of FIG. 1)

According to the methods of the present invention, the TILs are preselected for being PD-1 positive (PD-1+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are PD-1 positive (PD-1+) (for example, after preselection and before the priming first expansion). In some embodiments, TILs for use in the priming first expansion are at least 75% PD-1 positive, at least 80% PD-1 positive, at least 85% PD-1 positive, at least 90% PD-1 positive, at least 95% PD-1 positive, at least 98% PD-1 positive or at least 99% PD-1 positive (for example, after preselection and before the priming first expansion). In some embodiments, the PD-1 population is PD-1high. In some embodiments. TILs for use in the priming first expansion are at least 25% PD-1high, at least 30% PD-1high, at least 35% PD-1high, at least 40% PD-1high, at least 45% PD-1high, at least 50% PD-1high, at least 55% PD-1high, at least 60% PD-1high, at least 65% PD-1high, at least 70% PD-1high, at least 75% PD-1high, at least 80% PD-1high, at least 85% PD-1high, at least 90% PD-1high, at least 95% PD-1high, at least 98% PD-1high or at least 99% PD-1high (for example, after preselection and before the priming first expansion).

In some embodiments, PD-1high is indicated by a TIL population that is at least 75% PD-1 positive, at least 80% PD-1 positive, at least 85% PD-1 positive, at least 90% PD-1 positive, at least 95% PD-1 positive, at least 98% PD-1 positive or at least 99% PD-1 positive, or 100% PD-1 positive. In some embodiments, PD-1high is indicated by a TIL population that is at least 80% PD-1 positive. In some embodiments, PD-1high is indicated by a TIL population that is at least 85% PD-1 positive. In some embodiments, PD-1high is indicated by a TIL population that is at least 90% PD-1 positive. In some embodiments, PD-1high is indicated by a TIL population that is at least 95% PD-1 positive. In some embodiments, PD-1high is indicated by a TIL population that is at least 98% PD-1 positive. In some embodiments, PD-1high is indicated by a TIL population that is at least 99% PD-1 positive. In some embodiments, PD-1high is indicated by a TIL population that is 100% PD-1 positive.

In some embodiments, PD-1high is indicated by a TIL population wherein the TILs express at least 25% more PD-1 than a control or baseline PD-1 level, express at least 30% more PD-1 than a control or baseline PD-1 level, express at least 35% more PD-1 than a control or baseline PD-1 level, express at least 40% more PD-1 than a control or baseline PD-1 level, express at least 45% more PD-1 than a control or baseline PD-1 level, express at least 50% more PD-1 than a control or baseline PD-1 level, express at least 55% more PD-1 than a control or baseline PD-1 level, express at least 60% more PD-1 than a control or baseline PD-1 level, express at least 65% more PD-1 than a control or baseline PD-1 level, express at least 70% more PD-1 than a control or baseline PD-1 level, express at least 75% more PD-1 than a control or baseline PD-1 level, express at least 80% more PD-1 than a control or baseline PD-1 level, express at least 85% more PD-1 than a control or baseline PD-1 level, express at least 90% more PD-1 than a control or baseline PD-1 level, express at least 95% more PD-1 than a control or baseline PD-1 level, express at least 99% more PD-1 than a control or baseline PD-1 level, or express 100% more PD-1 than a control or baseline PD-1 level.

In some embodiments, PD-1high is indicated by a TIL population wherein the TILs express 1-fold or more PD-1 than a control or baseline PD-1 level. In some embodiments, PD-1high is indicated by a TIL population wherein the TILs express one-fold, two-fold, three-fold, four-fold, five-fold, ten-fold, or more PD-1 than a control or baseline PD-1 level. In some embodiments, PD-1high is indicated by a TIL population wherein the TILs express one-fold more PD-1 than a control or baseline PD-1 level. In some embodiments, PD-1high is indicated by a TIL population wherein the TILs express two-fold more PD-1 than a control or baseline PD-1 level. In some embodiments, PD-1high is indicated by a TIL population wherein the TILs express three-fold more PD-1 than a control or baseline PD-1 level. In some embodiments, PD-1high is indicated by a TIL population wherein the TILs express four-fold more PD-1 than a control or baseline PD-1 level. In some embodiments, PD-1high is indicated by a TIL population wherein the TILs express five-fold more PD-1 than a control or baseline PD-1 level. In some embodiments, PD-1high is indicated by a TIL population wherein the TILs express ten-fold more PD-1 than a control or baseline PD-1 level.

In some embodiments, the preselection of PD-1 positive TILs is performed by staining primary cell population, whole tumor digests, and/or whole tumor cell suspensions TILs with an anti-PD-1 antibody. In some embodiments, the anti-PD-1 antibody is a polyclonal antibody e.g., a mouse anti-human PD-1 polyclonal antibody, a goat anti-human PD-1 polyclonal antibody, etc. In some embodiments, the anti-PD-1 antibody is a monoclonal antibody. In some embodiments the anti-PD-1 antibody includes, e.g., but is not limited to EH12.2H7, PD1.3.1, SYM021, M1H4. A17188B, nivolumab (BMS-936558, Bristol-Myers Squibb; Opdivo®), pembrolizumab (lambrolizumab, MK03475 or MK-3475. Merck; Keytruda®), H12.1, PD1.3.1, NAT 105, humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat # BP0146. Other suitable antibodies for use in the preselection of PD-1 positive TILs for use in the expansion of TILs according to the methods of the invention, as exemplified by Steps A through F, as described herein are anti-PD-1 antibodies disclosed in U.S. Pat. No. 8,008,449, herein incorporated by reference. In some embodiments, the anti-PD-1 antibody for use in the preselection binds to a different epitope than nivolumab (BMS-936558, Bristol-Myers Squibb; Opdivo®). In some embodiments, the anti-PD-1 antibody for use in the preselection binds to a different epitope than pembrolizumab (lambrolizumab, MK03475 or MK-3475, Merck; Keytruda®). In some embodiments, the anti-PD-1 antibody for use in the preselection binds to a different epitope than humanized anti-PD-1 antibody JS001 (ShangHai JunShi). In some embodiments, the anti-PD-1 antibody for use in the preselection binds to a different epitope than monoclonal anti-PD-1 antibody TSR-042 (Tesaro, Inc.). In some embodiments, the anti-PD-1 antibody for use in the preselection binds to a different epitope than Pidilizumab (anti-PD-1 mAb CT-011, Medivation). In some embodiments, the anti-PD-1 antibody for use in the preselection binds to a different epitope than anti-PD-1 monoclonal Antibody BGB-A317 (BeiGene). In some embodiments, the anti-PD-1 antibody for use in the preselection binds to a different epitope than anti-PD-1 antibody SHR-1210 (ShangHai HengRui). In some embodiments, the anti-PD-1 antibody for use in the preselection binds to a different epitope than human monoclonal antibody REGN2810 (Regeneron). In some embodiments, the anti-PD-1 antibody for use in the preselection binds to a different epitope than human monoclonal antibody MDX-1106 (Bristol-Myers Squibb). In some embodiments, the anti-PD-1 antibody for use in the preselection binds to a different epitope than humanized anti-PD-1 IgG4 antibody PDR001 (Novartis). In some embodiments, the anti-PD-1 antibody for use in the preselection binds to a different epitope than RMP1-14 (rat IgG)—BioXcell cat #BP0146. The structures for binding of nivolumab and pembrolizumab binding to PD-1 are known and have been described in, for example, Tan, S. et al. (Tan, S. et al., Nature Communications, 8:14369|DOI: 10.1038/ncomms14369 (2017); incorporated by reference herein in its entirety for all purposes). In some embodiments, the anti-PD-1 antibody is EH12.2H7. In some embodiments, the anti-PD-1 antibody is PD1.3.1 In some embodiments, the anti-PD-1 antibody is not PD1.3.1 In some embodiments, the anti-PD-1 antibody is M1H4. In some embodiments, the anti-PD-1 antibody is not M1H4.

In some embodiments, the anti-PD-1 antibody for use in the preselection binds at least 75%, at least 80%, at least 85%, at least 90%, at least 95%, at least 98%, at least 99% or at least 100% of the cells expressing PD-1.

In some embodiments, the patient has been treated with an anti-PD-1 antibody. In some embodiments; the subject is anti-PD-1 antibody treatment naïve. In some embodiments, the subject has not been treated with an anti-PD-1 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-PD-1 antibody treatment. In some embodiments, the subject is post-chemotherapeutic treatment and post anti-PD-1 antibody treatment. In some embodiments, the patient is anti-PD-1 antibody treatment naïve. In some embodiments, the subject has treatment naïve cancer or is post-chemotherapeutic treatment but anti-PD-1 antibody treatment naïve. In some embodiments, the subject is treatment naïve and post-chemotherapeutic treatment but anti-PD-1 antibody treatment naive.

In some embodiments in which the patient has been previously treated with a first anti-PD-1 antibody, the preselection is performed by staining the primary cell population, whole tumor digests, and/or whole tumor cell suspensions TILs with a second anti-PD-1 antibody that is not blocked by the first anti-PD-1 antibody from binding to PD-1 on the surface of the primary cell population TILs.

In some embodiments in which the patient has been previously treated with an anti-PD-1 antibody, the preselection is performed by staining the primary cell population TILs with an antibody (an “anti-Fc antibody”) that binds to the Fc region of the anti-PD-1 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-PD-1 human or humanized IgG antibody, and the primary cell population TILs are stained with an anti-human IgG antibody. In some embodiments in which the patient has been previously treated with an anti-PD-1 human or humanized IgG1 antibody, the primary cell population TILs are stained with an anti-human IgG1 antibody. In some embodiments in which the patient has been previously treated with an anti-PD-1 human or humanized IgG2 antibody, the primary cell population TILs are stained with an anti-human IgG2 antibody. In some embodiments in which the patient has been previously treated with an anti-PD-1 human or humanized IgG3 antibody, the primary cell population TILs are stained with an anti-human IgG3 antibody. In some embodiments in which the patient has been previously treated with an anti-PD-1 human or humanized IgG4 antibody, the primary cell population TILs are stained with an anti-human IgG4 antibody.

In some embodiments in which the patient has been previously treated with an anti-PD-1 antibody, the preselection is performed by contacting the primary cell population TILs with the same anti-PD-1 antibody and then staining the primary cell population TILs with an anti-Fc antibody that binds to the Fc region of the anti-PD-1 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to PD-1 negative TILs, PD-1 intermediate TILs, and PD-1 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the PD-1high population is defined as the population of cells that is positive for PD-1 above what is observed in PBMCs. In some embodiments, the intermediate PD-1+ population in the TIL is encompasses the PD-1+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days, 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the PD-1 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%+0.25%. In some embodiments, the gating and compensation for the mean fluorescence intensity (MFI) is in the range of about 1.75%+0.25%. In some embodiments, the gating and compensation for the mean fluorescence intensity (MFI) is in the range of about 1.75%+0.25% when setting the PD-1 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of PD-1 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the PD-1 high gate. In some embodiments, the MFI calculation is the mean value measured using at least two samples of PBMCs. In some embodiments, the MFI calculation is the median value measured using at least two samples of PBMCs.

In some embodiments, the PD-1 positive (PD-1+) cells are sorted by FACs and/or other flow cytometry method. In some embodiments, the PD-1 positive TILs are PD-1high TILs. In some embodiments, the PD-1 positive TILs are PD-1intermediate TILs. In some embodiments, the PD-1+ cells are sorted by employing a bead selection method. In some embodiments, the PD-1+ cells are sorted by employing a magnetic bead selection method. In some embodiments, the bead selection employs an antibody bound bead, for example but no limited to a commercially available bead, such as Miltenyi or Fisher, for selection. In some embodiments, the PD-1+ high cells are sorted by employing a bead selection method. In some embodiments, the PD-1+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-PD-1 antibody is conjugated to the bead, either directly or indirectly. In some embodiments, the bead selection process selects for both PD-1+ and CD3+ TILs. In some embodiments the anti-PD-1 antibody employed in the bead selection method includes, e.g., but is not limited to EH12.2H7, PD1.3.1, SYM021, A17180, nivolumab (BMS-936558, Bristol-Myers Squibb; Opdivo®), pembrolizumab (lambrolizumab, MK03475 or MK-3475, Merck; Keytruda®), H12.1, PD1.3.1, NAT 105, humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable antibodies for use in the preselection of PD-1 positive TILs for use in the expansion of TILs according to the methods of the invention, as exemplified by Steps A through F, as described herein are anti-PD-1 antibodies disclosed in U.S. Pat. No. 8,008,449, herein incorporated by reference. In some embodiments, the anti-PD-1 antibody for use in the preselection binds to a different epitope than nivolumab (BMS-936558, Bristol-Myers Squibb; Opdivo®). In some embodiments, the anti-PD-1 antibody for use in the preselection binds to a different epitope than pembrolizumab (lambrolizumab, MK03475 or MK-3475, Merck; Keytruda®). In some embodiments, the anti-PD-1 antibody for use in the preselection binds to a different epitope than humanized anti-PD-1 antibody JS001 (ShangHai JunShi). In some embodiments, the anti-PD-1 antibody for use in the preselection binds to a different epitope than monoclonal anti-PD-1 antibody TSR-042 (Tesaro, Inc.). In some embodiments, the anti-PD-1 antibody for use in the preselection binds to a different epitope than Pidilizumab (anti-PD-1 mAb CT-011, Medivation). In some embodiments, the anti-PD-1 antibody for use in the preselection binds to a different epitope than anti-PD-1 monoclonal Antibody BGB-A317 (BeiGene). In some embodiments, the anti-PD-1 antibody for use in the preselection binds to a different epitope than anti-PD-1 antibody SHR-1210 (ShangHai HengRui). In some embodiments, the anti-PD-1 antibody for use in the preselection binds to a different epitope than human monoclonal antibody REGN2810 (Regeneron). In some embodiments, the anti-PD-1 antibody for use in the preselection binds to a different epitope than human monoclonal antibody MDX-1106 (Bristol-Myers Squibb). In some embodiments, the anti-PD-1 antibody for use in the preselection binds to a different epitope than humanized anti-PD-1 IgG4 antibody PDR001 (Novartis). In some embodiments, the anti-PD-1 antibody for use in the preselection binds to a different epitope than RMP1-14 (rat IgG)—BioXcell cat #BP0146. The structures for binding of nivolumab and pembrolizumab binding to PD-1 are known and have been described in, for example, Tan, S. et al. (Tan, S. et al., Nature Communications, 8:14369 DOI: 10.1038/ncomms14369 (2017); incorporated by reference herein in its entirety for all purposes). In some embodiments, the anti-PD-1 antibody is EH12.2H7. In some embodiments, the anti-PD-1 antibody is PD1.3.1 In some embodiments, the anti-PD-1 antibody is not PD1.3.1. In some embodiments, the anti-PD-1 antibody is M1H4. In some embodiments, the anti-PD-1 antibody is not M1H4.

In some embodiments, the collection buffer employed to collect the PD-1+ cells and/or the PD-1 negative cells does not include serum. In some embodiments, the collection buffer employed to collect the PD-1+ cells and/or the PD-1 negative cells includes serum. In some embodiments, the collection buffer employed to collect the PD-1+ cells and/or the PD-1 negative cells includes a component to mitigate or reduce viscosity differences between sort buffer and downstream buffers and/or media. In some embodiments, the collection buffer employed to collect the PD-1+ cells and/or the PD-1 negative cells includes only human serum albumin (HSA). In some embodiments, the collection buffer employed to collect the PD-1+ cells and/or the PD-1 negative cells includes an equal amount of HSA and PBS/EDTA Buffer. In some embodiments, the collection buffer employed to collect the PD-1+ cells and/or the PD-1 negative cells includes HSA and PBS/EDTA Buffer at a 1:1, 1:2, 1:3, 1:4, 2:1, 3:1, or 4:1 ratio.

In some embodiments, preselection involves selecting PD-1 positive TILs from the first population of TILs to obtain a PD-1 enriched TIL population comprises the selecting a population of TILs from a first population of TILs that are at least 11.27% to 74.4% PD-1 positive TILs. In some embodiments, the first population of TILs are at least 20% to 80% PD-1 positive TILs, at least 20% to 80% PD-1 positive TILs, at least 30% to 80% PD-1 positive TILs, at least 40% to 80% PD-1 positive TILs, at least 50% to 80% PD-1 positive TILs, at least 10% to 70% PD-1 positive TILs, at least 20% to 70% PD-1 positive TILs, at least 30% to 70% PD-1 positive TILs, or at least 40% to 70% PD-1 positive TILs.

In some embodiments, the selection step (e.g., preselection and/or selecting PD-1 positive cells) comprises the steps of:
(i) exposing the first population of TILs and a population of PBMC to an excess of a monoclonal anti-PD-1 IgG4 antibody that binds to PD-1 through an N-terminal loop outside the IgV domain of PD-1,
(ii) adding an excess of an anti-IgG4 antibody conjugated to a fluorophore,
(iii) obtaining the PD-1 enriched TIL population based on the intensity of the fluorophore of the PD-1 positive TILs in the first population of TILs compared to the intensity in the population of PBMCs as performed by fluorescence-activated cell sorting (FACS).

In some embodiments, the PD-1 positive TILs are PD-1high TILs.

In some embodiments, the PD-1high expression is determined by flow cytometry using minimum cutoff for normalized fluorescence intensity selected from the group consisting of about 80%, about 70%, about 60%, about 50%, about 40%, about 30%, about 20%, or about 10%.

In some embodiments, at least 70% of the PD-1 enriched TIL population are PD-1 positive TILs. In some embodiments, at least 80% of the PD-1 enriched TIL population are PD-1 positive TILs. In some embodiments, at least 90% of the PD-1 enriched TIL population are PD-1 positive TILs. In some embodiments, at least 95% of the PD-1 enriched TIL population are PD-1 positive TILs. In some embodiments, at least 99% of the PD-1 enriched TIL population are PD-1 positive TILs. In some embodiments, 100% of the PD-1 enriched TIL population are PD-1 positive TILs.

In some embodiments, the selection of PD-1 positive TILs occurs until there are at least 1×10⁴ TILs PD-1 positive TILs, at least 1×10⁵ TILs PD-1 positive TILs, at least 1×10⁶ TILs PD-1 positive TILs, at least 1×10⁷ TILs PD-1 positive TILs, at least 1×10⁸ TILS PD-1 positive TILs. In some embodiments, the selection of PD-1 positive TILs occurs until there are at least 1×10⁶ TILs PD-1 positive TILs.

Different anti-PD-1 antibodies exhibit different binding characteristics to different epitopes within PD-1. In some embodiments, the anti-PD-1 antibody binds to a different epitope than pembrolizumab. In some embodiments, the anti-PD1 antibody binds to an epitope in the N-terminal loop outside the IgV domain of PD-1. In some embodiments, the anti-PD1 antibody binds through an N-terminal loop outside the IgV domain of PD-1. In some embodiments, the anti-PD-1 anitbody is an anti-PD-1 antibody that binds to PD-1 binds through an N-terminal loop outside the IgV domain of PD-1. In some embodiments, the anti-PD-1 anitbody is a monoclonal anti-PD-1 antibody that binds to PD-1 binds through an N-terminal loop outside the IgV domain of PD-1. In some embodiments, the monoclonal anti-PD-1 anitbody is an anti-PD-1 IgG4 antibody that binds to PD-1 binds through an N-terminal loop outside the IgV domain of PD-1. See, for example, Tan, S. Nature Comm. Vol 8, Article 14369: 1-10 (2017).

In some embodiments, the selection step, exemplified as Step A2 of FIG. 1, comprises the steps of (i) exposing the first population of TILs to an excess of a monoclonal anti-PD-1 IgG4 antibody that binds to PD-1 through an N-terminal loop outside the IgV domain of PD-1, (ii) adding an excess of an anti-IgG4 antibody conjugated to a fluorophore, and (iii) performing a flow-based cell sort based on the fluorophore to obtain a PD-1 enriched TIL population. In some embodiments, the monoclonal anti-PD-1 IgG4 antibody is nivolumab or variants, fragments, or conjugates thereof. In some embodiments, the anti-IgG4 antibody is clone anti-human IgG4, Clone HP6023. In some embodiments, the anti-PD-1 antibody for use in the selection in step (b) binds to the same epitope as EH12.2H7 or nivolumab.

In some embodiments, the PD-1 gating method of WO2019156568 is employed. To determine if TILs derived from a tumor sample are PD-1high, one skilled in the art can utilize a reference value corresponding to the level of expression of PD-1 in peripheral T cells obtained from a blood sample from one or more healthy human subjects. PD-1 positive cells in the reference sample can be defined using fluorescence minus one controls and matching isotype controls. In some embodiments, the expression level of PD-1 is measured in CD3+/PD-1+ peripheral T cells from a healthy subject (e.g., the reference cells) is used to establish a threshold value or cut-off value of immunostaining intensity of PD-1 in TILs obtained from a tumor. The threshold value can be defined as the minimal intensity of PD-1 immunostaining of PD-1high T cells. As such, TILs with a PD-1 expression that is the same or above the threshold value can be considered to be PD-1high cells. In some instances, the PD-1high TILs represent those with the highest intensity of PD-1 immunostaining corresponding to a maximum 1% or less of the total CD3+ cells. In other instances, the PD-1high TILs represent those with the highest intensity of PD-1 immunostaining corresponding to the maximum 0.75% or less of the total CD3+ cells. In some instances, the PD-1high TILs represent those with the highest intensity of PD-1 immunostaining corresponding to the maximum 0.50% or less of the total CD3+ cells. In one instance, the PD-1high TILs represent those with the highest intensity of PD-1 immunostaining corresponding to the maximum 0.25% or less of the total CD3+ cells.

NSCLC patients. In brief, PD-1hi, PD-lint and PD-1neg subsets could be identified based on their measured fluorescence intensity.

In some embodiments, the PD-1 positive (PD-1+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-PD-1 antibody linked to a fluorophore and an anti-CD3 antibody linked to a fluorophore. In some embodiments, the primary cell population TILs are stained with a cocktail that includes an anti-PD-1 antibody linked to a fluorophore (for example, PE, live/dead violet) and anti-CD3-FITC. In some embodiments, the primary cell population TILs are stained with a cocktail that includes anti-PD-1-PE, anti-CD3-FITC and live/dead blue stain (ThermoFisher, MA, Cat #L23105). In some embodiments, the after incubation with the anti-PD1 antibody, PD-1 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

6. CD39—Preselection Selection for CD39 (as exemplified in Step A2 of FIG. 1)

According to the methods of the present invention, the TILs are preselected for being CD39 positive (CD39+) prior to the priming first expansion.

In some embodiments, the TILs of the present invention are preselected for an exhaustion marker such as CD39 (see, for example, Canale, F. P., et al. Cancer Res. 78:115-128 (2018) and or Duhne, T., et al., Nat Commun. 9:2724 (2018)). According to the methods of the present invention, the TILs are preselected for being CD39 positive (CD39+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are CD39 positive (CD39+) (for example, after preselection and before the priming first expansion). In some embodiments, TILs for use in the priming first expansion are at least 75% CD39 positive, at least 80% CD39 positive, at least 85% CD39positive, at least 90% CD39 positive, at least 95% CD39 positive, at least 98% CD39positive or at least 99% CD39positive (for example, after preselection and before the priming first expansion). In some embodiments, the CD39 population is CD39high. In some embodiments, TILs for use in the priming first expansion are at least 25% CD39high, at least 30% CD39high, at least 35% CD39high, at least 40% CD39high, at least 45% CD39high, at least 50% CD39high, at least 55% CD39high, at least 60% CD39high, at least 65% CD39high, at least 70% CD39high, at least 75% CD39high, at least 80% CD39high, at least 85% CD39high, at least 90% CD39high, at least 95% CD39high, at least 98% CD39high or at least 99% CD39high (for example, after preselection and before the priming first expansion).

In some embodiments, CD39high is indicated by a TIL population that is at least 75% CD39 positive, at least 80% CD39 positive, at least 85% CD39 positive, at least 90% CD39 positive, at least 95% CD39 positive, at least 98% CD39 positive or at least 99% CD39 positive, or 100% CD39 positive. In some embodiments, CD39high is indicated by a TIL population that is at least 80% CD39 positive. In some embodiments, CD39high is indicated by a TIL population that is at least 85% CD39 positive. In some embodiments, CD39high is indicated by a TIL population that is at least 90% CD39 positive. In some embodiments, CD39high is indicated by a TIL population that is at least 95% CD39 positive. In some embodiments, CD39high is indicated by a TIL population that is at least 98% CD39 positive. In some embodiments, CD39high is indicated by a TIL population that is at least 99% CD39 positive. In some embodiments, CD39high is indicated by a TIL population that is 100% CD39 positive.

In some embodiments, CD39high is indicated by a TIL population wherein the TILs express at least 25% more CD39 than a control or baseline CD39 level, express at least 30% more CD39 than a control or baseline CD39 level, express at least 35% more CD39 than a control or baseline CD39 level, express at least 40% more CD39 than a control or baseline CD39 level, express at least 45% more CD39 than a control or baseline CD39 level, express at least 50% more CD39 than a control or baseline CD39 level, express at least 55% more CD39 than a control or baseline CD39 level, express at least 60% more CD39 than a control or baseline CD39 level, express at least 65% more CD39 than a control or baseline CD39 level, express at least 70% more CD39 than a control or baseline CD39 level, express at least 75% more CD39 than a control or baseline CD39 level, express at least 80% more CD39 than a control or baseline CD39 level, express at least 85% more CD39 than a control or baseline CD39 level, express at least 90% more CD39 than a control or baseline CD39 level, express at least 95% more CD39 than a control or baseline CD39 level, express at least 99% more CD39 than a control or baseline CD39 level, or express 100% more CD39 than a control or baseline CD39 level.

In some embodiments, CD39high is indicated by a TIL population wherein the TILs express 1-fold or more CD39 than a control or baseline CD39 level. In some embodiments, CD39high is indicated by a TIL population wherein the TILs express one-fold, two-fold, three-fold, four-fold, five-fold, ten-fold, or more CD39 than a control or baseline CD39 level. In some embodiments, CD39high is indicated by a TIL population wherein the TILs express one-fold more CD39 than a control or baseline CD39 level. In some embodiments, CD39high is indicated by a TIL population wherein the TILs express two-fold more CD39 than a control or baseline CD39 level. In some embodiments. CD39high is indicated by a TIL population wherein the TILs express three-fold more CD39 than a control or baseline CD39 level. In some embodiments, CD39high is indicated by a TIL population wherein the TILs express four-fold more CD39 than a control or baseline CD39 level. In some embodiments, CD39high is indicated by a TIL population wherein the TILs express five-fold more CD39 than a control or baseline CD39 level. In some embodiments, CD39high is indicated by a TIL population wherein the TILs express ten-fold more CD39 than a control or baseline CD39 level.

In some embodiments, the preselection of CD39 positive TILs is performed by staining primary cell population, whole tumor digests, and/or whole tumor cell suspensions TILs with an anti-CD39 antibody. In some embodiments, the anti-CD39 antibody is a polyclonal antibody e.g., a mouse anti-human CD39 polyclonal antibody, a goat anti-human CD39 polyclonal antibody, etc. In some embodiments, the anti-CD39 antibody is a monoclonal antibody. In some embodiments the anti-CD39 antibody includes, e.g., but is not limited to BY40 (See, Nikolova, M., et al. PLoS Pathog. 7, e1002110 (2011)), IPH5201, TTX-0303, SRF617, and/or 5F2.

In some embodiments, the anti-CD39 antibody for use in the preselection binds at least 75%, at least 80%, at least 85%, at least 90%, at least 95%, at least 98%, at least 99% or at least 100% of the cells expressing CD39.

In some embodiments, the patient has been treated with an anti-CD39 antibody. In some embodiments, the subject is anti-CD39 antibody treatment naïve. In some embodiments, the subject has not been treated with an anti-CD39 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-CD39 antibody treatment. In some embodiments, the subject is post-chemotherapeutic treatment and post anti-CD39 antibody treatment. In some embodiments, the patient is anti-CD39 antibody treatment naïve. In some embodiments, the subject has treatment naïve cancer or is post-chemotherapeutic treatment but anti-CD39 antibody treatment naïve. In some embodiments, the subject is treatment naïve and post-chemotherapeutic treatment but anti-CD39 antibody treatment naive.

In some embodiments in which the patient has been previously treated with a first anti-CD39 antibody, the preselection is performed by staining the primary cell population, whole tumor digests, and/or whole tumor cell suspensions TILs with a second anti-CD39 antibody that is not blocked by the first anti-CD39 antibody from binding to CD39 on the surface of the primary cell population TILs.

In some embodiments in which the patient has been previously treated with an anti-CD39 antibody, the preselection is performed by staining the primary cell population TILs with an antibody (an “anti-Fc antibody”) that binds to the Fc region of the anti-CD39 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-CD39 human or humanized IgG antibody, and the primary cell population TILs are stained with an anti-human IgG antibody. In some embodiments in which the patient has been previously treated with an anti-CD39 human or humanized IgG1 antibody, the primary cell population TILs are stained with an anti-human IgG1 antibody. In some embodiments in which the patient has been previously treated with an anti-CD39 human or humanized IgG2 antibody, the primary cell population TILs are stained with an anti-human IgG2 antibody. In some embodiments in which the patient has been previously treated with an anti-CD39 human or humanized IgG3 antibody, the primary cell population TILs are stained with an anti-human IgG3 antibody. In some embodiments in which the patient has been previously treated with an anti-CD39 human or humanized IgG4 antibody, the primary cell population TILs are stained with an anti-human IgG4 antibody.

In some embodiments in which the patient has been previously treated with an anti-CD39 antibody, the preselection is performed by contacting the primary cell population TILs with the same anti-CD39 antibody and then staining the primary cell population TILs with an anti-Fc antibody that binds to the Fc region of the anti-CD39 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to CD39 negative TILs, CD39 intermediate TILs, and CD39 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the CD39high population is defined as the population of cells that is positive for CD39 above what is observed in PBMCs. In some embodiments, the intermediate CD39+ population in the TIL is encompasses the CD39+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days, 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the CD39 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%±0.25%. In some embodiments, the gating and compensation for the mean fluorescence intensity (MFI) is in the range of about 1.75%±0.25%. In some embodiments, the gating and compensation for the mean fluorescence intensity (MFI) is in the range of about L75%±0.25% when setting the CD39 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of CD39 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the CD high gate. In some embodiments, the MFI calculation is the mean value measured using at least two samples of PBMCs. In some embodiments, the MFI calculation is the median value measured using at least two samples of PBMCs.

In some embodiments, the CD39 positive (CD39+) cells are sorted by FACs and/or other flow cytometry method. In some embodiments, the CD39 positive TILs are CD39high TILs. In some embodiments, the CD39 positive TILs are CD39intermediate TILs. In some embodiments, the CD39+ cells are sorted by employing a bead selection method. In some embodiments, the CD39+ cells are sorted by employing a magnetic bead selection method. In some embodiments, the bead selection employs an antibody bound bead, for example but no limited to a commercially available bead, such as Miltenyi or Fisher, for selection. In some embodiments, the CD39+ high cells are sorted by employing a bead selection method. In some embodiments, the CD39+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-CD39 antibody is conjugated to the bead, either directly or indirectly. In some embodiments, the bead selection process selects for both CD39+ and CD3+ TILs. In some embodiments the anti-CD39 antibody employed in the bead selection method includes, e.g., but is not limited to BY40 (See, Nikolova, M., et al. PLoS Pathog. 7, e1002110 (2011)), IPH5201, TTX-0303, SRF617, and/or 5F2.

In some embodiments, the collection buffer employed to collect the CD39+ cells and/or the CD39 negative cells does not include serum. In some embodiments, the collection buffer employed to collect the CD39+ cells and/or the CD39 negative cells includes serum. In some embodiments, the collection buffer employed to collect the CD39+ cells and/or the CD39 negative cells includes a component to mitigate or reduce viscosity differences between sort buffer and downstream buffers and/or media. In some embodiments, the collection buffer employed to collect the CD39+ cells and/or the CD39 negative cells includes only human serum albumin (HSA). In some embodiments, the collection buffer employed to collect the CD39+ cells and/or the CD39 negative cells includes an equal amount of HSA and PBS/EDTA Buffer. In some embodiments, the collection buffer employed to collect the CD39+ cells and/or the CD39 negative cells includes HSA and PBS/EDTA Buffer at a 1:1, 1:2, 1:3, 1:4, 2:1, 3:1, or 4:1 ratio.

In some embodiments, preselection involves selecting CD39 positive TILs from the first population of TILs to obtain a CD39 enriched TIL population comprises the selecting a population of TILs from a first population of TILs that are at least 11.27% to 74.4% CD39 positive TILs. In some embodiments, the first population of TILs are at least 20% to 80% CD39 positive TILs, at least 20% to 80% CD39 positive TILs, at least 30% to 80% CD39 positive TILs, at least 40% to 80% CD39 positive TILs, at least 50% to 80% CD39 positive TILs, at least 10% to 70% CD39 positive TILs, at least 20% to 70% CD39 positive TILs, at least 30% to 70% CD39 positive TILs, or at least 40% to 70% CD39 positive TILs.

In some embodiments, the selection step (e.g., preselection and/or selecting CD39 positive cells) comprises the steps of:
(i) exposing the first population of TILs and a population of PBMC to an excess of a monoclonal anti-CD39 IgG4 antibody that binds to CD39 through an N-terminal loop outside the IgV domain of CD39,
(ii) adding an excess of an anti-IgG4 antibody conjugated to a fluorophore,
(iii) obtaining the CD39 enriched TIL population based on the intensity of the fluorophore of the CD39 positive TILs in the first population of TILs compared to the intensity in the population of PBMCs as performed by fluorescence-activated cell sorting (FACS).

In some embodiments, the CD39 positive TILs are CD39high TILs.

In some embodiments, at least 70% of the CD39 enriched TIL population are CD39 positive TILs. In some embodiments, at least 80% of the CD39 enriched TIL population are CD39 positive TILs. In some embodiments, at least 90% of the CD39 enriched TIL population are CD39 positive TILs. In some embodiments, at least 95% of the CD39 enriched TIL population are CD39 positive TILs. In some embodiments, at least 99% of the CD39 enriched TIL population are CD39 positive TILs. In some embodiments, 100% of the CD39 enriched TIL population are CD39 positive TILs.

In some embodiments, the selection of CD39 positive TILs occurs until there are at least 1×10⁴ TILs CD39 positive TILs, at least 1×10⁵ TILs CD39 positive TILs, at least 1×10⁶ TILs CD39 positive TILs, at least 1×10⁷ TILs CD39 positive TILs, at least 1×10⁸ TILs CD39 positive TILs. In some embodiments, the selection of CD39 positive TILs occurs until there are at least 1×10⁶ TILs CD39 positive TILs.

In some embodiments, the selection step, exemplified as Step A2 of FIG. 1, comprises the steps of (i) exposing the first population of TILs to an excess of a monoclonal anti-CD39 IgG4 antibody that binds to CD39 through an N-terminal loop outside the IgV domain of CD39, (ii) adding an excess of an anti-IgG4 antibody conjugated to a fluorophore, and (iii) performing a flow-based cell sort based on the fluorophore to obtain a CD39 enriched TIL population. In some embodiments, the monoclonal anti-CD39 IgG4 antibody is nivolumab or variants, fragments, or conjugates thereof. In some embodiments, the anti-IgG4 antibody is clone anti-human IgG4, Clone HP6023. In some embodiments, the anti-CD39 antibody for use in the selection in step (b) binds to the same epitope as EH12.2H7 or nivolumab.

To determine if TILs derived from a tumor sample are CD39high, one skilled in the art can utilize a reference value corresponding to the level of expression of CD39 in peripheral T cells obtained from a blood sample from one or more healthy human subjects. CD39 positive cells in the reference sample can be defined using fluorescence minus one controls and matching isotype controls. In some embodiments, the expression level of CD39 is measured in CD3+/CD39+ peripheral T cells from a healthy subject (e.g., the reference cells) is used to establish a threshold value or cut-off value of immunostaining intensity of CD39 in TILs obtained from a tumor. The threshold value can be defined as the minimal intensity of CD39 immunostaining of CD39high T cells. As such, TILs with a CD39 expression that is the same or above the threshold value can be considered to be CD39high cells. In some instances, the CD39high TILs represent those with the highest intensity of CD39 immunostaining corresponding to a maximum 1% or less of the total CD3+ cells. In other instances, the CD39high TILs represent those with the highest intensity of CD39 immunostaining corresponding to the maximum 0.75% or less of the total CD3+ cells. In some instances, the CD39high TILs represent those with the highest intensity of CD39 immunostaining corresponding to the maximum 0.50% or less of the total CD3+ cells. In one instance, the CD39high TILs represent those with the highest intensity of CD39 immunostaining corresponding to the maximum 0.25% or less of the total CD3+ cells.

In some embodiments, the PD-1 gating method of WO2019156568 is employed for CD39. To determine if TILs derived from a tumor sample are CD39high, one skilled in the art can utilize a reference value corresponding to the level of expression of CD39 in peripheral T cells obtained from a blood sample from one or more healthy human subjects. CD39 positive cells in the reference sample can be defined using fluorescence minus one controls and matching isotype controls. In some embodiments, the expression level of CD39 is measured in CD3+/CD39+ peripheral T cells from a healthy subject (e.g., the reference cells) is used to establish a threshold value or cut-off value of immunostaining intensity of CD39 in TILs obtained from a tumor. The threshold value can be defined as the minimal intensity of CD39 immunostaining of CD39high T cells. As such, TILs with a CD39 expression that is the same or above the threshold value can be considered to be CD39high cells. In some instances, the CD39high TILs represent those with the highest intensity of CD39 immunostaining corresponding to a maximum 1% or less of the total CD3+ cells. In other instances, the CD39high TILs represent those with the highest intensity of CD39 immunostaining corresponding to the maximum 0.75% or less of the total CD3+ cells. In some instances, the CD39high TILs represent those with the highest intensity of CD39 immunostaining corresponding to the maximum 0.50% or less of the total CD3+ cells. In one instance, the CD39high TILs represent those with the highest intensity of CD39 immunostaining corresponding to the maximum 0.25% or less of the total CD3+ cells.

In some embodiments, the CD39 positive (CD39+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-CD39 antibody linked to a fluorophore and an anti-CD3 antibody linked to a fluorophore. In some embodiments, the primary cell population TILs are stained with a cocktail that includes an anti-CD39 antibody linked to a fluorophore (for example, PE, live/dead violet) and anti-CD3-FITC. In some embodiments, the primary cell population TILs are stained with a cocktail that includes anti-CD39-PE, anti-CD3-FITC and live/dead blue stain (ThermoFisher, MA, Cat #L23105). In some embodiments, the after incubation with the anti-PD1 antibody, CD39 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

7. CD38—Preselection Selection for CD38 (as exemplified in Step A2 of FIG. 1)

According to the methods of the present invention, the TILs are preselected for being CD38 positive (CD38+) prior to the priming first expansion.

In some embodiments, the TILs of the present invention are preselected for an exhaustion marker such as CD38 (see, for example, Canale, F. P., et al. Cancer Res. 78:115-128 (2018) and or Duhne, T., et al., Nat Commun. 9:2724 (2018)). According to the methods of the present invention, the TILs are preselected for being CD38 positive (CD38+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments, the TILs for use in the priming first expansion are CD38 positive (CD38+) (for example, after preselection and before the priming first expansion). In some embodiments, TILs for use in the priming first expansion are at least 75% CD38 positive, at least 80% CD38 positive, at least 85% CD38positive, at least 90% CD38 positive, at least 95% CD38 positive, at least 98% CD38positive or at least 99% CD38positive (for example, after preselection and before the priming first expansion). In some embodiments, the CD38 population is CD38 low (CD38lo). In some embodiments, TILs for use in the priming first expansion are at least 25% CD38lo, at least 30% CD38lo, at least 35% CD38lo, at least 40% CD38lo, at least 45% CD38lo, at least 50% CD38lo, at least 55% CD38lo, at least 60% CD38lo, at least 65% CD38lo, at least 70% CD38lo, at least 75% CD38lo, at least 80% CD38lo, at least 85% CD38lo, at least 90% CD38lo, at least 95% CD38lo, at least 98% CD38lo or at least 99% CD38lo (for example, after preselection and before the priming first expansion).

In some embodiments, CD38lo is indicated by a TIL population that is no more than 5% CD38 positive, no more than 10% CD38 positive, no more than 15% CD38 positive, no more than 20% CD38 positive, no more than 25% CD38 positive, no more than 30% CD38 positive, 35% CD38 positive, no more than 40% CD38 positive, no more than 45% CD38 positive, no more than 50% CD38 positive, no more than 55% CD38 positive, no more than 60% CD38 positive. In some embodiments, CD38lo is indicated by a TIL population that is no more than 5% CD38 positive. In some embodiments, CD38lo is indicated by a TIL population that is no more than 10% CD38 positive. In some embodiments, CD38lo is indicated by a TIL population that is no more than 15% CD38 positive. In some embodiments, CD38lo is indicated by a TIL population that is no more than 20% CD38 positive. In some embodiments, CD38lo is indicated by a TIL population that is no more than 25% CD38 positive. In some embodiments, CD38lo is indicated by a TIL population that is no more than 30% CD38 positive.

In some embodiments, CD38lo is indicated by a TIL population wherein the TILs express 25% less CD38 as compared to a control or baseline CD38 level, express 30% less CD38 as compared to a control or baseline CD38 level, express 35% less CD38 as compared to a control or baseline CD38 level, express 40% less CD38 as compared to a control or baseline CD38 level, express 45% less CD38 as compared to a control or baseline CD38 level, express 50% less CD38 as compared to a control or baseline CD38 level, express 55% less CD38 as compared to a control or baseline CD38 level, express 60% less CD38 as compared to a control or baseline CD38 level, express 65% less CD38 as compared to a control or baseline CD38 level, express 70% less CD38 as compared to a control or baseline CD38 level, express 75% less CD38 as compared to a control or baseline CD38 level, express 80% less CD38 as compared to a control or baseline CD38 level, express 85% less CD38 as compared to a control or baseline CD38 level, express 90% less CD38 as compared to a control or baseline CD38 level, express 95% less CD38 as compared to a control or baseline CD38 level, or express 99% less CD38 as compared to a control or baseline CD38 level.

In some embodiments, CD38lo is indicated by a TIL population wherein the TILs express 1-fold or less CD38 than a control or baseline CD38 level. In some embodiments, CD38lo is indicated by a TIL population wherein the TILs express one-fold, two-fold, three-fold, four-fold, five-fold, ten-fold, or less CD38 than a control or baseline CD38 level. In some embodiments, CD38lo is indicated by a TIL population wherein the TILs express one-fold less CD38 than a control or baseline CD38 level. In some embodiments, CD38lo is indicated by a TIL population wherein the TILs express two-fold less CD38 than a control or baseline CD38 level. In some embodiments, CD38lo is indicated by a TIL population wherein the TILs express three-fold less CD38 than a control or baseline CD38 level. In some embodiments, CD38lo is indicated by a TIL population wherein the TILs express four-fold less CD38 than a control or baseline CD38 level. In some embodiments, CD38lo is indicated by a TIL population wherein the TILs express five-fold less CD38 than a control or baseline CD38 level. In some embodiments, CD38lo is indicated by a TIL population wherein the TILs express ten-fold less CD38 than a control or baseline CD38 level.

In some embodiments the TILs for use in the priming first expansion are CD38 positive (CD38+) (for example, after preselection and before the priming first expansion). In some embodiments, TILs for use in the priming first expansion are at least 75% CD38 positive, at least 80% CD38 positive, at least 85% CD38positive, at least 90% CD38 positive, at least 95% CD38 positive, at least 98% CD38positive or at least 99% CD38positive (for example, after preselection and before the priming first expansion). In some embodiments, the CD38 population is CD38high. In some embodiments, TILs for use in the priming first expansion are at least 25% CD38high, at least 30% CD38high, at least 35% CD38high, at least 40% CD38high, at least 45% CD38high, at least 50% CD38high, at least 55% CD38high, at least 60% CD38high, at least 65% CD38high, at least 70% CD38high, at least 75% CD38high, at least 80% CD38high, at least 85% CD38high, at least 90% CD38high, at least 95% CD38high, at least 98% CD38high or at least 99% CD38high (for example, after preselection and before the priming first expansion).

In some embodiments, CD38high is indicated by a TIL population that is at least 75% CD38 positive, at least 80% CD38 positive, at least 85% CD38 positive, at least 90% CD38 positive, at least 95% CD38 positive, at least 98% CD38 positive or at least 99% CD38 positive, or 100% CD38 positive. In some embodiments, CD38high is indicated by a TIL population that is at least 80% CD38 positive. In some embodiments, CD38high is indicated by a TIL population that is at least 85% CD38 positive. In some embodiments, CD38high is indicated by a TIL population that is at least 90% CD38 positive. In some embodiments, CD38high is indicated by a TIL population that is at least 95% CD38 positive. In some embodiments, CD38high is indicated by a TIL population that is at least 98% CD38 positive. In some embodiments, CD38high is indicated by a TIL population that is at least 99% CD38 positive. In some embodiments, CD38high is indicated by a TIL population that is 100% CD38 positive.

In some embodiments, CD38high is indicated by a TIL population wherein the TILs express at least 25% more CD38 than a control or baseline CD38 level, express at least 30% more CD38 than a control or baseline CD38 level, express at least 35% more CD38 than a control or baseline CD38 level, express at least 40% more CD38 than a control or baseline CD38 level, express at least 45% more CD38 than a control or baseline CD38 level, express at least 50% more CD38 than a control or baseline CD38 level, express at least 55% more CD38 than a control or baseline CD38 level, express at least 60% more CD38 than a control or baseline CD38 level, express at least 65% more CD38 than a control or baseline CD38 level, express at least 70% more CD38 than a control or baseline CD38 level, express at least 75% more CD38 than a control or baseline CD38 level, express at least 80% more CD38 than a control or baseline CD38 level, express at least 85% more CD38 than a control or baseline CD38 level, express at least 90% more CD38 than a control or baseline CD38 level, express at least 95% more CD38 than a control or baseline CD38 level, express at least 99% more CD38 than a control or baseline CD38 level, or express 100% more CD38 than a control or baseline CD38 level.

In some embodiments, CD38high is indicated by a TIL population wherein the TILs express 1-fold or more CD38 than a control or baseline CD38 level. In some embodiments, CD38high is indicated by a TIL population wherein the TILs express one-fold, two-fold, three-fold, four-fold, five-fold, ten-fold, or more CD38 than a control or baseline CD38 level. In some embodiments, CD38high is indicated by a TIL population wherein the TILs express one-fold more CD38 than a control or baseline CD38 level. In some embodiments, CD38high is indicated by a TIL population wherein the TILs express two-fold more CD38 than a control or baseline CD38 level. In some embodiments, CD38high is indicated by a TIL population wherein the TILs express three-fold more CD38 than a control or baseline CD38 level. In some embodiments, CD38high is indicated by a TIL population wherein the TILs express four-fold more CD38 than a control or baseline CD38 level. In some embodiments, CD38high is indicated by a TIL population wherein the TILs express five-fold more CD38 than a control or baseline CD38 level. In some embodiments, CD38high is indicated by a TIL population wherein the TILs express ten-fold more CD38 than a control or baseline CD38 level.

In some embodiments, the preselection of CD38 positive TILs is performed by staining primary cell population, whole tumor digests, and/or whole tumor cell suspensions TILs with an anti-CD38 antibody. In some embodiments, the anti-CD38 antibody is a polyclonal antibody e.g., a mouse anti-human CD38 polyclonal antibody, a goat anti-human CD38 polyclonal antibody, etc. In some embodiments, the anti-CD38 antibody is a monoclonal antibody. In some embodiments the anti-CD38 antibody includes, e.g., but is not limited to MOR03087, Daratumumab, GSK2857916, MOR202, STI-6129, Isatuximab (SAR650984), and/or TAK-079

In some embodiments, the anti-CD38 antibody for use in the preselection binds at least 75%, at least 80%, at least 85%, at least 90%, at least 95%, at least 98%, at least 99% or at least 100% of the cells expressing CD38.

In some embodiments, the patient has been treated with an anti-CD38 antibody. In some embodiments, the subject is anti-CD38 antibody treatment naïve. In some embodiments, the subject has not been treated with an anti-CD38 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-CD38 antibody treatment. In some embodiments, the subject is post-chemotherapeutic treatment and post anti-CD38 antibody treatment. In some embodiments, the patient is anti-CD38 antibody treatment naïve. In some embodiments, the subject has treatment naïve cancer or is post-chemotherapeutic treatment but anti-CD38 antibody treatment naïve. In some embodiments, the subject is treatment naïve and post-chemotherapeutic treatment but anti-CD38 antibody treatment naive.

In some embodiments in which the patient has been previously treated with a first anti-CD38 antibody, the preselection is performed by staining the primary cell population, whole tumor digests, and/or whole tumor cell suspensions TILs with a second anti-CD38 antibody that is not blocked by the first anti-CD38 antibody from binding to CD38 on the surface of the primary cell population TILs.

In some embodiments in which the patient has been previously treated with an anti-CD38 antibody, the preselection is performed by staining the primary cell population TILs with an antibody (an “anti-Fc antibody”) that binds to the Fc region of the anti-CD38 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-CD38 human or humanized IgG antibody, and the primary cell population TILs are stained with an anti-human IgG antibody. In some embodiments in which the patient has been previously treated with an anti-CD38 human or humanized IgG1 antibody, the primary cell population TILs are stained with an anti-human IgG1 antibody. In some embodiments in which the patient has been previously treated with an anti-CD38 human or humanized IgG2 antibody, the primary cell population TILs are stained with an anti-human IgG2 antibody. In some embodiments in which the patient has been previously treated with an anti-CD38 human or humanized IgG3 antibody, the primary cell population TILs are stained with an anti-human IgG3 antibody. In some embodiments in which the patient has been previously treated with an anti-CD38 human or humanized IgG4 antibody, the primary cell population TILs are stained with an anti-human IgG4 antibody.

In some embodiments in which the patient has been previously treated with an anti-CD38 antibody, the preselection is performed by contacting the primary cell population TILs with the same anti-CD38 antibody and then staining the primary cell population TILs with an anti-Fc antibody that binds to the Fc region of the anti-CD38 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to CD38 negative TILs, CD38 intermediate TILs, and CD38 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the CD38high population is defined as the population of cells that is positive for CD38 above what is observed in PBMCs. In some embodiments, the intermediate CD38+ population in the TIL is encompasses the CD38+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days, 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the CD38 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%+0.25%. In some embodiments, the gating and compensation for the mean fluorescence intensity (MFI) is in the range of about 1.75%+0.25%. In some embodiments, the gating and compensation for the mean fluorescence intensity (MFI) is in the range of about 1.75%+0.25% when setting the CD38 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of CD38 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the CD38 high gate. In some embodiments, the MFI calculation is the mean value measured using at least two samples of PBMCs. In some embodiments, the MFI calculation is the median value measured using at least two samples of PBMCs.

In some embodiments, the CD38 positive (CD38+) cells are sorted by FACs and/or other flow cytometry method. In some embodiments, the CD38 positive TILs are CD38high TILs. In some embodiments, the CD38 positive TILs are CD38intermediate TILs. In some embodiments, the CD38+ cells are sorted by employing a bead selection method. In some embodiments, the CD38+ cells are sorted by employing a magnetic bead selection method. In some embodiments, the bead selection employs an antibody bound bead, for example but no limited to a commercially available bead, such as Miltenyi or Fisher, for selection. In some embodiments, the CD38+ high cells are sorted by employing a bead selection method. In some embodiments, the CD38+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-CD38 antibody is conjugated to the bead, either directly or indirectly. In some embodiments, the bead selection process selects for both CD38+ and CD3+ TILs. In some embodiments the anti-CD38 antibody employed in the bead selection method includes, e.g., but is not limited to M0R03087, Daratumumab, GSK2857916, MOR202, STI-6129, Isatuximab (SAR650984), and/or TAK-079.

In some embodiments, the collection buffer employed to collect the CD38+ cells and/or the CD38 negative cells does not include serum. In some embodiments, the collection buffer employed to collect the CD38+ cells and/or the CD38 negative cells includes serum. In some embodiments, the collection buffer employed to collect the CD38+ cells and/or the CD38 negative cells includes a component to mitigate or reduce viscosity differences between sort buffer and downstream buffers and/or media. In some embodiments, the collection buffer employed to collect the CD38+ cells and/or the CD38 negative cells includes only human serum albumin (HSA). In some embodiments, the collection buffer employed to collect the CD38+ cells and/or the CD38 negative cells includes an equal amount of HSA and PBS/EDTA Buffer. In some embodiments, the collection buffer employed to collect the CD38+ cells and/or the CD38 negative cells includes HSA and PBS/EDTA Buffer at a 1:1, 1:2, 1:3, 1:4, 2:1, 3:1, or 4:1 ratio.

In some embodiments, preselection involves selecting CD38 positive TILs from the first population of TILs to obtain a CD38 enriched TIL population comprises the selecting a population of TILs from a first population of TILs that are at least 11.27% to 74.4% CD38 positive TILs. In some embodiments, the first population of TILs are at least 20% to 80% CD38 positive TILs, at least 20% to 80% CD38 positive TILs, at least 30% to 80% CD38 positive TILs, at least 40% to 80% CD38 positive TILs, at least 50% to 80% CD38 positive TILs, at least 10% to 70% CD38 positive TILs, at least 20% to 70% CD38 positive TILs, at least 30% to 70% CD38 positive TILs, or at least 40% to 70% CD38 positive TILs.

In some embodiments, the selection step (e.g., preselection and/or selecting CD38 positive cells) comprises the steps of:
(i) exposing the first population of TILs and a population of PBMC to an excess of a monoclonal anti-CD38 IgG4 antibody that binds to CD38 through an N-terminal loop outside the IgV domain of CD38,
(ii) adding an excess of an anti-IgG4 antibody conjugated to a fluorophore,
(iii) obtaining the CD38 enriched TIL population based on the intensity of the fluorophore of the CD38 positive TILs in the first population of TILs compared to the intensity in the population of PBMCs as performed by fluorescence-activated cell sorting (FACS).

In some embodiments, the CD38 positive TILs are CD38high TILs.

In some embodiments, at least 70% of the CD38 enriched TIL population are CD38 positive TILs. In some embodiments, at least 80% of the CD38 enriched TIL population are CD38 positive TILs. In some embodiments, at least 90% of the CD38 enriched TIL population are CD38 positive TILs. In some embodiments, at least 95% of the CD38 enriched TIL population are CD38 positive TILs. In some embodiments, at least 99% of the CD38 enriched TIL population are CD38 positive TILs. In some embodiments, 100% of the CD38 enriched TIL population are CD38 positive TILs.

In some embodiments, the selection of CD38 positive TILs occurs until there are at least 1×10⁴ TILs CD38 positive TILs, at least 1×10⁵ TILs CD38 positive TILs, at least 1×10⁶ TILs CD38 positive TILs, at least 1×10⁷ TILs CD38 positive TILs, at least 1×10⁸ TILS CD38 positive TILs. In some embodiments, the selection of CD38 positive TILs occurs until there are at least 1×10⁶ TILs CD38 positive TILs.

In some embodiments, the selection step, exemplified as Step A2 of FIG. 1, comprises the steps of (i) exposing the first population of TILs to an excess of a monoclonal anti-CD38 IgG4 antibody that binds to CD38 through an N-terminal loop outside the IgV domain of CD38, (ii) adding an excess of an anti-IgG4 antibody conjugated to a fluorophore, and (iii) performing a flow-based cell sort based on the fluorophore to obtain a CD38 enriched TIL population. In some embodiments, the monoclonal anti-CD38 IgG4 antibody is nivolumab or variants, fragments, or conjugates thereof. In some embodiments, the anti-IgG4 antibody is clone anti-human IgG4, Clone HP6023. In some embodiments, the anti-CD38 antibody for use in the selection in step (b) binds to the same epitope as EH12.2H7 or nivolumab.

To determine if TILs derived from a tumor sample are CD38high, one skilled in the art can utilize a reference value corresponding to the level of expression of CD38 in peripheral T cells obtained from a blood sample from one or more healthy human subjects. CD38 positive cells in the reference sample can be defined using fluorescence minus one controls and matching isotype controls. In some embodiments, the expression level of CD38 is measured in CD3+/CD38+ peripheral T cells from a healthy subject (e.g., the reference cells) is used to establish a threshold value or cut-off value of immunostaining intensity of CD38 in TILs obtained from a tumor. The threshold value can be defined as the minimal intensity of CD38 immunostaining of CD38high T cells. As such, TILs with a CD38 expression that is the same or above the threshold value can be considered to be CD38high cells. In some instances, the CD38high TILs represent those with the highest intensity of CD38 immunostaining corresponding to a maximum 1% or less of the total CD3+ cells. In other instances, the CD38high TILs represent those with the highest intensity of CD38 immunostaining corresponding to the maximum 0.75% or less of the total CD3+ cells. In some instances, the CD38high TILs represent those with the highest intensity of CD38 immunostaining corresponding to the maximum 0.50% or less of the total CD3+ cells. In one instance, the CD38high TILs represent those with the highest intensity of CD38 immunostaining corresponding to the maximum 0.25% or less of the total CD3+ cells.

In some embodiments, the PD-1 gating method of WO2019156568 is employed for CD38. To determine if TILs derived from a tumor sample are CD38high, one skilled in the art can utilize a reference value corresponding to the level of expression of CD38 in peripheral T cells obtained from a blood sample from one or more healthy human subjects. CD38 positive cells in the reference sample can be defined using fluorescence minus one controls and matching isotype controls. In some embodiments, the expression level of CD38 is measured in CD3+/CD38+ peripheral T cells from a healthy subject (e.g., the reference cells) is used to establish a threshold value or cut-off value of immunostaining intensity of CD38 in TILs obtained from a tumor. The threshold value can be defined as the minimal intensity of CD38 immunostaining of CD38high T cells. As such, TILs with a CD38 expression that is the same or above the threshold value can be considered to be CD38high cells. In some instances, the CD38high TILs represent those with the highest intensity of CD38 immunostaining corresponding to a maximum 1% or less of the total CD3+ cells. In other instances, the CD38high TILs represent those with the highest intensity of CD38 immunostaining corresponding to the maximum 0.75% or less of the total CD3+ cells. In some instances, the CD38high TILs represent those with the highest intensity of CD38 immunostaining corresponding to the maximum 0.50% or less of the total CD3+ cells. In one instance, the CD38high TILs represent those with the highest intensity of CD38 immunostaining corresponding to the maximum 0.25% or less of the total CD3+ cells.

In some embodiments, the CD38 positive (CD38+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-CD38 antibody linked to a fluorophore and an anti-CD3 antibody linked to a fluorophore. In some embodiments, the primary cell population TILs are stained with a cocktail that includes an anti-CD38antibody linked to a fluorophore (for example, PE, live/dead violet) and anti-CD3-FITC. In some embodiments, the primary cell population TILs are stained with a cocktail that includes anti-CD38-PE, anti-CD3-FITC and live/dead blue stain (ThermoFisher, MA, Cat #L23105). In some embodiments, the after incubation with the anti-CD38 antibody, CD38 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to; tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

8. CD103—Preselection Selection for CD103 (as Exemplified in Step A2 of FIG. 1)

According to the methods of the present invention, the TILs are preselected for being CD103 positive (CD103+) prior to the priming first expansion.

In some embodiments, the TILs of the present invention are preselected for an exhaustion marker such as CD103 (also know as αeβ7 or αEβ7, see, for example, or Duhne, T., et al., Nat Commun. 9:2724 (2018)). According to the methods of the present invention, the TILs are preselected for being CD103 positive (CD103+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are CD103 positive (CD103+) (for example, after preselection and before the priming first expansion). In some embodiments, TILs for use in the priming first expansion are at least 75% CD103 positive, at least 80% CD103 positive, at least 85% CD103positive, at least 90% CD103 positive, at least 95% CD103 positive, at least 98% CD103positive or at least 99% CD103positive (for example, after preselection and before the priming first expansion). In some embodiments, the CD103 population is CD103high. In some embodiments, TILs for use in the priming first expansion are at least 25% CD103high, at least 30% CD103high, at least 35% CD103high, at least 40% CD103high, at least 45% CD103high, at least 50% CD103high, at least 55% CD103high, at least 60% CD103high, at least 65% CD103high, at least 70% CD103high, at least 75% CD103high, at least 80% CD103high, at least 85% CD103high, at least 90% CD103high, at least 95% CD103high, at least 98% CD103high or at least 99% CD103high (for example, after preselection and before the priming first expansion).

In some embodiments, CD103high is indicated by a TIL population that is at least 75% CD103 positive, at least 80% CD103 positive, at least 85% CD103 positive, at least 90% CD103 positive, at least 95% CD103 positive, at least 98% CD103 positive or at least 99% CD103 positive, or 100% CD103 positive. In some embodiments, CD103high is indicated by a TIL population that is at least 80% CD103 positive. In some embodiments, CD103high is indicated by a TIL population that is at least 85% CD103 positive. In some embodiments, CD103high is indicated by a TIL population that is at least 90% CD103 positive. In some embodiments, CD103high is indicated by a TIL population that is at least 95% CD103 positive. In some embodiments, CD103high is indicated by a TIL population that is at least 98% CD103 positive. In some embodiments, CD103high is indicated by a TIL population that is at least 99% CD103 positive. In some embodiments, CD103high is indicated by a TIL population that is 100% CD103 positive.

In some embodiments, CD103high is indicated by a TIL population wherein the TILs express at least 25% more CD103 than a control or baseline CD103 level, express at least 30% more CD103 than a control or baseline CD103 level, express at least 35% more CD103 than a control or baseline CD103 level, express at least 40% more CD103 than a control or baseline CD103 level, express at least 45% more CD103 than a control or baseline CD103 level, express at least 50% more CD103 than a control or baseline CD103 level, express at least 55% more CD103 than a control or baseline CD103 level, express at least 60% more CD103 than a control or baseline CD103 level, express at least 65% more CD103 than a control or baseline CD103 level, express at least 70% more CD103 than a control or baseline CD103 level, express at least 75% more CD103 than a control or baseline CD103 level, express at least 80% more CD103 than a control or baseline CD103 level, express at least 85% more CD103 than a control or baseline CD103 level, express at least 90% more CD103 than a control or baseline CD103 level, express at least 95% more CD103 than a control or baseline CD103 level, express at least 99% more CD103 than a control or baseline CD103 level, or express 100% more CD103 than a control or baseline CD103 level.

In some embodiments, CD103high is indicated by a TIL population wherein the TILs express 1-fold or more CD103 than a control or baseline CD103 level. In some embodiments, CD103high is indicated by a TIL population wherein the TILs express one-fold, two-fold, three-fold, four-fold, five-fold, ten-fold, or more CD103 than a control or baseline CD103 level. In some embodiments, CD103high is indicated by a TIL population wherein the TILs express one-fold more CD103 than a control or baseline CD103 level. In some embodiments, CD103high is indicated by a TIL population wherein the TILs express two-fold more CD103 than a control or baseline CD103 level. In some embodiments, CD103high is indicated by a TIL population wherein the TILs express three-fold more CD103 than a control or baseline CD103 level. In some embodiments, CD103high is indicated by a TIL population wherein the TILs express four-fold more CD103 than a control or baseline CD103 level. In some embodiments, CD103high is indicated by a TIL population wherein the TILs express five-fold more CD103 than a control or baseline CD103 level. In some embodiments, CD103high is indicated by a TIL population wherein the TILs express ten-fold more CD103 than a control or baseline CD103 level.

In some embodiments, the preselection of CD103 positive TILs is performed by staining primary cell population, whole tumor digests, and/or whole tumor cell suspensions TILs with an anti-CD103 antibody. In some embodiments, the anti-CD103 antibody is a polyclonal antibody e.g., a mouse anti-human CD103 polyclonal antibody, a goat anti-human CD103 polyclonal antibody, etc. In some embodiments, the anti-CD103 antibody is a monoclonal antibody. In some embodiments the anti-CD103 antibody includes, e.g., but is not limited to APC (17-1031-82), Ber-ACT8, and/or M290.

In some embodiments, the anti-CD103 antibody for use in the preselection binds at least 75%, at least 80%, at least 85%, at least 90%, at least 95%, at least 98%, at least 99% or at least 100% of the cells expressing CD103.

In some embodiments, the patient has been treated with an anti-CD103 antibody. In some embodiments, the subject is anti-CD103 antibody treatment naïve. In some embodiments, the subject has not been treated with an anti-CD103 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-CD103 antibody treatment. In some embodiments, the subject is post-chemotherapeutic treatment and post anti-CD103 antibody treatment. In some embodiments, the patient is anti-CD103 antibody treatment naïve. In some embodiments, the subject has treatment naïve cancer or is post-chemotherapeutic treatment but anti-CD103 antibody treatment naïve. In some embodiments, the subject is treatment naïve and post-chemotherapeutic treatment but anti-CD103 antibody treatment naive.

In some embodiments in which the patient has been previously treated with a first anti-CD103 antibody, the preselection is performed by staining the primary cell population, whole tumor digests, and/or whole tumor cell suspensions TILs with a second anti-CD103 antibody that is not blocked by the first anti-CD103 antibody from binding to CD103 on the surface of the primary cell population TILs.

In some embodiments in which the patient has been previously treated with an anti-CD103 antibody, the preselection is performed by staining the primary cell population TILs with an antibody (an “anti-Fc antibody”) that binds to the Fc region of the anti-CD103 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-CD103 human or humanized IgG antibody, and the primary cell population TILs are stained with an anti-human IgG antibody. In some embodiments in which the patient has been previously treated with an anti-CD103 human or humanized IgG1 antibody, the primary cell population TILs are stained with an anti-human IgG1 antibody. In some embodiments in which the patient has been previously treated with an anti-CD103 human or humanized IgG2 antibody, the primary cell population TILs are stained with an anti-human IgG2 antibody. In some embodiments in which the patient has been previously treated with an anti-CD103 human or humanized IgG3 antibody, the primary cell population TILs are stained with an anti-human IgG3 antibody. In some embodiments in which the patient has been previously treated with an anti-CD103 human or humanized IgG4 antibody, the primary cell population TILs are stained with an anti-human IgG4 antibody.

In some embodiments in which the patient has been previously treated with an anti-CD103 antibody, the preselection is performed by contacting the primary cell population TILs with the same anti-CD103 antibody and then staining the primary cell population TILs with an anti-Fc antibody that binds to the Fc region of the anti-CD103 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to CD103 negative TILs, CD103 intermediate TILs, and CD103 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the CD103high population is defined as the population of cells that is positive for CD103 above what is observed in PBMCs. In some embodiments, the intermediate CD103+ population in the TIL is encompasses the CD103+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days, 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the CD103 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%+0.25%. In some embodiments, the gating and compensation for the mean fluorescence intensity (MFI) is in the range of about 1.75%+0.25%. In some embodiments, the gating and compensation for the mean fluorescence intensity (MFI) is in the range of about L75%+0.25% when setting the CD103 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of CD103 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the CD103 high gate. In some embodiments, the MFI calculation is the mean value measured using at least two samples of PBMCs. In some embodiments, the MFI calculation is the median value measured using at least two samples of PBMCs.

In some embodiments, the CD103 positive (CD103+) cells are sorted by FACs and/or other flow cytometry method. In some embodiments, the CD103 positive TILs are CD103high TILs. In some embodiments, the CD103 positive TILs are CD103intermediate TILs. In some embodiments, the CD103+ cells are sorted by employing a bead selection method. In some embodiments, the CD103+ cells are sorted by employing a magnetic bead selection method. In some embodiments, the bead selection employs an antibody bound bead, for example but no limited to a commercially available bead, such as Miltenyi or Fisher, for selection. In some embodiments, the CD103+ high cells are sorted by employing a bead selection method. In some embodiments, the CD103+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-CD103 antibody is conjugated to the bead, either directly or indirectly. In some embodiments, the bead selection process selects for both CD103+ and CD3+ TILs. In some embodiments the anti-CD103 antibody employed in the bead selection method includes, e.g., but is not limited to APC (17-1031-82), Ber-ACTS, and/or M290.

In some embodiments, the collection buffer employed to collect the CD103+ cells and/or the CD103 negative cells does not include serum. In some embodiments, the collection buffer employed to collect the CD103+ cells and/or the CD103 negative cells includes serum. In some embodiments, the collection buffer employed to collect the CD103+ cells and/or the CD103 negative cells includes a component to mitigate or reduce viscosity differences between sort buffer and downstream buffers and/or media. In some embodiments, the collection buffer employed to collect the CD103+ cells and/or the CD103 negative cells includes only human serum albumin (HSA). In some embodiments, the collection buffer employed to collect the CD103+ cells and/or the CD103 negative cells includes an equal amount of HSA and PBS/EDTA Buffer. In some embodiments, the collection buffer employed to collect the CD103+ cells and/or the CD103 negative cells includes HSA and PBS/EDTA Buffer at a 1:1, 1:2, 1:3, 1:4, 2:1, 3:1, or 4:1 ratio.

In some embodiments, preselection involves selecting CD103 positive TILs from the first population of TILs to obtain a CD103 enriched TIL population comprises the selecting a population of TILs from a first population of TILs that are at least 11.27% to 74.4% CD103 positive TILs. In some embodiments, the first population of TILs are at least 20% to 80% CD103 positive TILs, at least 20% to 80% CD103 positive TILs, at least 30% to 80% CD103 positive TILs, at least 40% to 80% CD103 positive TILs, at least 50% to 80% CD103 positive TILs, at least 10% to 70% CD103 positive TILs, at least 20% to 70% CD103 positive TILs, at least 30% to 70% CD103 positive TILs, or at least 40% to 70% CD103 positive TILs.

In some embodiments, the selection step (e.g., preselection and/or selecting CD103 positive cells) comprises the steps of:
(i) exposing the first population of TILs and a population of PBMC to an excess of a monoclonal anti-CD103 IgG4 antibody that binds to CD103 through an N-terminal loop outside the IgV domain of CD103,
(ii) adding an excess of an anti-IgG4 antibody conjugated to a fluorophore,
(iii) obtaining the CD103 enriched TIL population based on the intensity of the fluorophore of the CD103 positive TILs in the first population of TILs compared to the intensity in the population of PBMCs as performed by fluorescence-activated cell sorting (FACS).

In some embodiments, the CD103 positive TILs are CD103high TILs.

In some embodiments, at least 70% of the CD103 enriched TIL population are CD103 positive TILs. In some embodiments, at least 80% of the CD103 enriched TIL population are CD103 positive TILs. In some embodiments, at least 90% of the CD103 enriched TIL population are CD103 positive TILs. In some embodiments, at least 95% of the CD103 enriched TIL population are CD103 positive TILs. In some embodiments, at least 99% of the CD103 enriched TIL population are CD103 positive TILs. In some embodiments, 100% of the CD103 enriched TIL population are CD103 positive TILs.

In some embodiments, the selection of CD103 positive TILs occurs until there are at least 1×10⁴ TILs CD103 positive TILs, at least 1×10⁵ TILs CD103 positive TILs, at least 1×10⁶ TILs CD103 positive TILs, at least 1×10⁷ TILs CD103 positive TILs, at least 1×10⁸ TILs CD103 positive TILs. In some embodiments, the selection of CD103 positive TILs occurs until there are at least 1×10⁶ TILs CD103 positive TILs.

In some embodiments, the selection step, exemplified as Step A2 of FIG. 1, comprises the steps of (i) exposing the first population of TILs to an excess of a monoclonal anti-CD103 IgG4 antibody that binds to CD103 through an N-terminal loop outside the IgV domain of CD103, (ii) adding an excess of an anti-IgG4 antibody conjugated to a fluorophore, and (iii) performing a flow-based cell sort based on the fluorophore to obtain a CD103 enriched TIL population. In some embodiments, the monoclonal anti-CD103 IgG4 antibody is nivolumab or variants, fragments, or conjugates thereof. In some embodiments, the anti-IgG4 antibody is clone anti-human IgG4, Clone HP6023. In some embodiments, the anti-CD103 antibody for use in the selection in step (b) binds to the same epitope as EH12.2H7 or nivolumab.

To determine if TILs derived from a tumor sample are CD103high, one skilled in the art can utilize a reference value corresponding to the level of expression of CD103 in peripheral T cells obtained from a blood sample from one or more healthy human subjects. CD103 positive cells in the reference sample can be defined using fluorescence minus one controls and matching isotype controls. In some embodiments, the expression level of CD103 is measured in CD3+/CD103+ peripheral T cells from a healthy subject (e.g., the reference cells) is used to establish a threshold value or cut-off value of immunostaining intensity of CD103 in TILs obtained from a tumor. The threshold value can be defined as the minimal intensity of CD103 immunostaining of CD103high T cells. As such, TILs with a CD103 expression that is the same or above the threshold value can be considered to be CD103high cells. In some instances, the CD103high TILs represent those with the highest intensity of CD103 immunostaining corresponding to a maximum 1% or less of the total CD3+ cells. In other instances, the CD103high TILs represent those with the highest intensity of CD103 immunostaining corresponding to the maximum 0.75% or less of the total CD3+ cells. In some instances, the CD103high TILs represent those with the highest intensity of CD103 immunostaining corresponding to the maximum 0.50% or less of the total CD3+ cells. In one instance, the CD103high TILs represent those with the highest intensity of CD103 immunostaining corresponding to the maximum 0.25% or less of the total CD3+ cells.

In some embodiments, the PD-1 gating method of WO2019156568 is employed for CD103. To determine if TILs derived from a tumor sample are CD103high, one skilled in the art can utilize a reference value corresponding to the level of expression of CD103 in peripheral T cells obtained from a blood sample from one or more healthy human subjects. CD103 positive cells in the reference sample can be defined using fluorescence minus one controls and matching isotype controls. In some embodiments, the expression level of CD103 is measured in CD3+/CD103+ peripheral T cells from a healthy subject (e.g., the reference cells) is used to establish a threshold value or cut-off value of immunostaining intensity of CD103 in TILs obtained from a tumor. The threshold value can be defined as the minimal intensity of CD103 immunostaining of CD103high T cells. As such, TILs with a CD103 expression that is the same or above the threshold value can be considered to be CD103high cells. In some instances, the CD103high TILs represent those with the highest intensity of CD103 immunostaining corresponding to a maximum 1% or less of the total CD3+ cells. In other instances, the CD103high TILs represent those with the highest intensity of CD103 immunostaining corresponding to the maximum 0.75% or less of the total CD3+ cells. In some instances, the CD103high TILs represent those with the highest intensity of CD103 immunostaining corresponding to the maximum 0.50% or less of the total CD3+ cells. In one instance, the CD103high TILs represent those with the highest intensity of CD103 immunostaining corresponding to the maximum 0.25% or less of the total CD3+ cells.

In some embodiments, the CD103 positive (CD103+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-CD103 antibody linked to a fluorophore and an anti-CD3 antibody linked to a fluorophore. In some embodiments, the primary cell population TILs are stained with a cocktail that includes an anti-CD103 antibody linked to a fluorophore (for example, PE, live/dead violet) and anti-CD3-FITC. In some embodiments, the primary cell population TILs are stained with a cocktail that includes anti-CD103-PE, anti-CD3-FITC and live/dead blue stain (ThermoFisher; MA, Cat #L23105). In some embodiments, the after incubation with the anti-PD1 antibody, CD103 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

9. CD101—Preselection Selection for CD101 (as exemplified in Step A2 of FIG. 1)

According to the methods of the present invention, the TILs are preselected for being CD101 positive (CD101+) prior to the priming first expansion.

In some embodiments, the TILs of the present invention are preselected for an exhaustion marker such as CD101 (Philip, M., et al., Nature. 545(7655):452-456 (2017)). According to the methods of the present invention, the TILs are preselected for being CD101 positive (CD101+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are CD101 positive (CD101+) (for example, after preselection and before the priming first expansion). In some embodiments, TILs for use in the priming first expansion are at least 75% CD101 positive, at least 80% CD101 positive, at least 85% CD101positive, at least 90% CD101 positive, at least 95% CD101 positive, at least 98% CD101positive or at least 99% CD101positive (for example, after preselection and before the priming first expansion). In some embodiments, the CD101 population is CD101high. In some embodiments, TILs for use in the priming first expansion are at least 25% CD101high, at least 30% CD101high, at least 35% CD101high, at least 40% CD101high, at least 45% CD101high, at least 50% CD101high, at least 55% CD101high, at least 60% CD101high, at least 65% CD101high, at least 70% CD101high, at least 75% CD101high, at least 80% CD101high, at least 85% CD101high, at least 90% CD101high, at least 95% CD101high, at least 98% CD101high or at least 99% CD101high (for example, after preselection and before the priming first expansion).

In some embodiments, the TILs for use in the priming first expansion are CD101 positive (CD101+) (for example, after preselection and before the priming first expansion). In some embodiments, TILs for use in the priming first expansion are at least 75% CD101 positive, at least 80% CD101 positive, at least 85% CD101positive, at least 90% CD101 positive, at least 95% CD101 positive, at least 98% CD101positive or at least 99% CD101positive (for example, after preselection and before the priming first expansion). In some embodiments, the CD101 population is CD101 low (CD101lo). In some embodiments, TILs for use in the priming first expansion are at least 25% CD101lo, at least 30% CD101lo, at least 35% CD101lo, at least 40% CD101lo, at least 45% CD101lo, at least 50% CD101lo, at least 55% CD101lo, at least 60% CD101lo, at least 65% CD101lo, at least 70% CD101lo, at least 75% CD101lo, at least 80% CD101lo, at least 85% CD101lo, at least 90% CD101lo, at least 95% CD101lo, at least 98% CD101lo or at least 99% CD10110 (for example, after preselection and before the priming first expansion).

In some embodiments, CD101lo is indicated by a TIL population that is no more than 5% CD101 positive, no more than 10% CD101 positive, no more than 15% CD101 positive, no more than 20% CD101 positive, no more than 25% CD101 positive, no more than 30% CD101 positive, 35% CD101 positive, no more than 40% CD101 positive, no more than 45% CD101 positive, no more than 50% CD101 positive, no more than 55% CD101 positivₑ; no more than 60% CD101 positive. In some embodiments, CD101lo is indicated by a TIL population that is no more than 5% CD101 positive. In some embodiments, CD101lo is indicated by a TIL population that is no more than 10% CD101 positive. In some embodiments, CD101lo is indicated by a TIL population that is no more than 15% CD101 positive. In some embodiments, CD101lo is indicated by a TIL population that is no more than 20% CD101 positive. In some embodiments, CD101lo is indicated by a TIL population that is no more than 25% CD101 positive. In some embodiments, CD101lo is indicated by a TIL population that is no more than 30% CD101 positive.

In some embodiments, CD101lo is indicated by a TIL population wherein the TILs express 25% less CD101 as compared to a control or baseline CD101 level, express 30% less CD101 as compared to a control or baseline CD101 level, express express 35% less CD101 as compared to a control or baseline CD101 level, express 40% less CD101 as compared to a control or baseline CD101 level, express 45% less CD101 as compared to a control or baseline CD101 level, express 50% less CD101 as compared to a control or baseline CD101 level, express 55% less CD101 as compared to a control or baseline CD101 level, express 60% less CD101 as compared to a control or baseline CD101 level, express 65% less CD101 as compared to a control or baseline CD101 level, express 70% less CD101 as compared to a control or baseline CD101 level, express 75% less CD101 as compared to a control or baseline CD101 level, express 80% less CD101 as compared to a control or baseline CD101 level, express 85% less CD101 as compared to a control or baseline CD101 level, express 90% less CD101 as compared to a control or baseline CD101 level, express 95% less CD101 as compared to a control or baseline CD101 level, or express 99% less CD101 as compared to a control or baseline CD101 level.

In some embodiments, CD101lo is indicated by a TIL population wherein the TILs express 1-fold or less CD101 than a control or baseline CD101 level. In some embodiments, CD10110 is indicated by a TIL population wherein the TILs express one-fold, two-fold, three-fold, four-fold, five-fold, ten-fold, or less CD101 than a control or baseline CD101 level. In some embodiments, CD101lo is indicated by a TIL population wherein the TILs express one-fold less CD101 than a control or baseline CD101 level. In some embodiments, CD101lo is indicated by a TIL population wherein the TILs express two-fold less CD101 than a control or baseline CD101 level. In some embodiments, CD10110 is indicated by a TIL population wherein the TILs express three-fold less CD101 than a control or baseline CD101 level. In some embodiments, CD101lo is indicated by a TIL population wherein the TILs express four-fold less CD101 than a control or baseline CD101 level. In some embodiments, CD101lo is indicated by a TIL population wherein the TILs express five-fold less CD101 than a control or baseline CD101 level. In some embodiments, CD101lo is indicated by a TIL population wherein the TILs express ten-fold less CD101 than a control or baseline CD101 level.

In some embodiments, CD101high is indicated by a TIL population that is at least 75% CD101 positive, at least 80% CD101 positive, at least 85% CD101 positive, at least 90% CD101 positive, at least 95% CD101 positive, at least 98% CD101 positive or at least 99% CD101 positive, or 100% CD101 positive. In some embodiments, CD101high is indicated by a TIL population that is at least 80% CD101 positive. In some embodiments, CD101high is indicated by a TIL population that is at least 85% CD101 positive. In some embodiments, CD101high is indicated by a TIL population that is at least 90% CD101 positive. In some embodiments, CD101high is indicated by a TIL population that is at least 95% CD101 positive. In some embodiments, CD101high is indicated by a TIL population that is at least 98% CD101 positive. In some embodiments, CD101high is indicated by a TIL population that is at least 99% CD101 positive. In some embodiments. CD101high is indicated by a TIL population that is 100% CD101 positive.

In some embodiments, CD101high is indicated by a TIL population wherein the TILs express at least 25% more CD101 than a control or baseline CD101 level, express at least 30% more CD101 than a control or baseline CD101 level, express at least 35% more CD101 than a control or baseline CD101 level, express at least 40% more CD101 than a control or baseline CD101 level, express at least 45% more CD101 than a control or baseline CD101 level, express at least 50% more CD101 than a control or baseline CD101 level, express at least 55% more CD101 than a control or baseline CD101 level, express at least 60% more CD101 than a control or baseline CD101 level, express at least 65% more CD101 than a control or baseline CD101 level, express at least 70% more CD101 than a control or baseline CD101 level, express at least 75% more CD101 than a control or baseline CD101 level, express at least 80% more CD101 than a control or baseline CD101 level, express at least 85% more CD101 than a control or baseline CD101 level, express at least 90% more CD101 than a control or baseline CD101 level, express at least 95% more CD101 than a control or baseline CD101 level, express at least 99% more CD101 than a control or baseline CD101 level, or express 100% more CD101 than a control or baseline CD101 level.

In some embodiments, CD101high is indicated by a TIL population wherein the TILs express 1-fold or more CD101 than a control or baseline CD101 level. In some embodiments, CD101high is indicated by a TIL population wherein the TILs express one-fold, two-fold, three-fold, four-fold, five-fold, ten-fold, or more CD101 than a control or baseline CD101 level. In some embodiments, CD101high is indicated by a TIL population wherein the TILs express one-fold more CD101 than a control or baseline CD101 level. In some embodiments, CD101high is indicated by a TIL population wherein the TILs express two-fold more CD101 than a control or baseline CD101 level. In some embodiments, CD101high is indicated by a TIL population wherein the TILs express three-fold more CD101 than a control or baseline CD101 level. In some embodiments, CD101high is indicated by a TIL population wherein the TILs express four-fold more CD101 than a control or baseline CD101 level. In some embodiments, CD101high is indicated by a TIL population wherein the TILs express five-fold more CD101 than a control or baseline CD101 level. In some embodiments, CD101high is indicated by a TIL population wherein the TILs express ten-fold more CD101 than a control or baseline CD101 level.

In some embodiments, the preselection of CD101 positive TILs is performed by staining primary cell population, whole tumor digests, and/or whole tumor cell suspensions TILs with an anti-CD101 antibody. In some embodiments, the anti-CD101 antibody is a polyclonal antibody e.g., a mouse anti-human CD101 polyclonal antibody, a goat anti-human CD101 polyclonal antibody, etc. In some embodiments, the anti-CD101 antibody is a monoclonal antibody. In some embodiments the anti-CD101 antibody includes, e.g., but is not limited to BB27.

In some embodiments, the anti-CD101 antibody for use in the preselection binds at least 75%, at least 80%, at least 85%, at least 90%, at least 95%, at least 98%, at least 99% or at least 100% of the cells expressing CD101.

In some embodiments, the patient has been treated with an anti-CD101 antibody. In some embodiments, the subject is anti-CD101 antibody treatment naïve. In some embodiments, the subject has not been treated with an anti-CD101 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-CD101 antibody treatment. In some embodiments, the subject is post-chemotherapeutic treatment and post anti-CD101 antibody treatment. In some embodiments, the patient is anti-CD101 antibody treatment naïve. In some embodiments, the subject has treatment naïve cancer or is post-chemotherapeutic treatment but anti-CD101 antibody treatment naïve. In some embodiments, the subject is treatment naïve and post-chemotherapeutic treatment but anti-CD101 antibody treatment naive.

In some embodiments in which the patient has been previously treated with a first anti-CD101 antibody, the preselection is performed by staining the primary cell population, whole tumor digests, and/or whole tumor cell suspensions TILs with a second anti-CD101 antibody that is not blocked by the first anti-CD101 antibody from binding to CD101 on the surface of the primary cell population TILs.

In some embodiments in which the patient has been previously treated with an anti-CD101 antibody, the preselection is performed by staining the primary cell population TILs with an antibody (an “anti-Fc antibody”) that binds to the Fc region of the anti-CD101 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-CD101 human or humanized IgG antibody, and the primary cell population TILs are stained with an anti-human IgG antibody. In some embodiments in which the patient has been previously treated with an anti-CD101 human or humanized IgG1 antibody, the primary cell population TILs are stained with an anti-human IgG1 antibody. In some embodiments in which the patient has been previously treated with an anti-CD101 human or humanized IgG2 antibody, the primary cell population TILs are stained with an anti-human IgG2 antibody. In some embodiments in which the patient has been previously treated with an anti-CD101 human or humanized IgG3 antibody, the primary cell population TILs are stained with an anti-human IgG3 antibody. In some embodiments in which the patient has been previously treated with an anti-CD101 human or humanized IgG4 antibody, the primary cell population TILs are stained with an anti-human IgG4 antibody.

In some embodiments in which the patient has been previously treated with an anti-CD101 antibody, the preselection is performed by contacting the primary cell population TILs with the same anti-CD101 antibody and then staining the primary cell population TILs with an anti-Fc antibody that binds to the Fc region of the anti-CD101 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to CD101 negative TILs, CD101 intermediate TILs, and CD101 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the CD101high population is defined as the population of cells that is positive for CD101 above what is observed in PBMCs. In some embodiments, the intermediate CD101+ population in the TIL is encompasses the CD101+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days. 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the CD101 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%+0.25%. In some embodiments, the gating and compensation for the mean fluorescence intensity (MFI) is in the range of about 1.75%+0.25%. In some embodiments, the gating and compensation for the mean fluorescence intensity (MFI) is in the range of about L75%+0.25% when setting the CD101 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of CD101 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the CD101 high gate. In some embodiments, the MFI calculation is the mean value measured using at least two samples of PBMCs. In some embodiments, the MFI calculation is the median value measured using at least two samples of PBMCs.

In some embodiments, the CD101 positive (CD101+) cells are sorted by FACs and/or other flow cytometry method. In some embodiments, the CD101 positive TILs are CD101high TILs. In some embodiments, the CD101 positive TILs are CD101intermediate TILs. In some embodiments, the CD101+ cells are sorted by employing a bead selection method. In some embodiments, the CD101+ cells are sorted by employing a magnetic bead selection method. In some embodiments, the bead selection employs an antibody bound bead, for example but no limited to a commercially available bead, such as Miltenyi or Fisher, for selection. In some embodiments, the CD101+ high cells are sorted by employing a bead selection method. In some embodiments, the CD101+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-CD101 antibody is conjugated to the bead, either directly or indirectly. In some embodiments, the bead selection process selects for both CD101+ and CD3+ TILs. In some embodiments the anti-CD101 antibody employed in the bead selection method includes, e.g., but is not limited to BB27

In some embodiments, the collection buffer employed to collect the CD101+ cells and/or the CD101 negative cells does not include serum. In some embodiments, the collection buffer employed to collect the CD101+ cells and/or the CD101 negative cells includes serum. In some embodiments, the collection buffer employed to collect the CD101+ cells and/or the CD101 negative cells includes a component to mitigate or reduce viscosity differences between sort buffer and downstream buffers and/or media. In some embodiments, the collection buffer employed to collect the CD101+ cells and/or the CD101 negative cells includes only human serum albumin (HSA). In some embodiments, the collection buffer employed to collect the CD101+ cells and/or the CD101 negative cells includes an equal amount of HSA and PBS/EDTA Buffer. In some embodiments, the collection buffer employed to collect the CD101+ cells and/or the CD101 negative cells includes HSA and PBS/EDTA Buffer at a 1:1, 1:2, 1:3, 1:4, 2:1, 3:1, or 4:1 ratio.

In some embodiments, preselection involves selecting CD101 positive TILs from the first population of TILs to obtain a CD101 enriched TIL population comprises the selecting a population of TILs from a first population of TILs that are at least 11.27% to 74.4% CD101 positive TILs. In some embodiments, the first population of TILs are at least 20% to 80% CD101 positive TILs, at least 20% to 80% CD101 positive TILs, at least 30% to 80% CD101 positive TILs, at least 40% to 80% CD101 positive TILs, at least 50% to 80% CD101 positive TILs, at least 10% to 70% CD101 positive TILs, at least 20% to 70% CD101 positive TILs, at least 30% to 70% CD101 positive TILs, or at least 40% to 70% CD101 positive TILs.

In some embodiments, the selection step (e.g., preselection and/or selecting CD101 positive cells) comprises the steps of:
(i) exposing the first population of TILs and a population of PBMC to an excess of a monoclonal anti-CD101 IgG4 antibody that binds to CD101 through an N-terminal loop outside the IgV domain of CD101,
(ii) adding an excess of an anti-IgG4 antibody conjugated to a fluorophore,
(iii) obtaining the CD101 enriched TIL population based on the intensity of the fluorophore of the CD101 positive TILs in the first population of TILs compared to the intensity in the population of PBMCs as performed by fluorescence-activated cell sorting (FACS).

In some embodiments, the CD101 positive TILs are CD101high TILs.

In some embodiments, at least 70% of the CD101 enriched TIL population are CD101 positive TILs. In some embodiments, at least 80% of the CD101 enriched TIL population are CD101 positive TILs. In some embodiments, at least 90% of the CD101 enriched TIL population are CD101 positive TILs. In some embodiments, at least 95% of the CD101 enriched TIL population are CD101 positive TILs. In some embodiments, at least 99% of the CD101 enriched TIL population are CD101 positive TILs. In some embodiments, 100% of the CD101 enriched TIL population are CD101 positive TILs.

In some embodiments, the selection of CD101 positive TILs occurs until there are at least 1×10⁴ TILs CD101 positive TILs, at least 1×10⁵ TILs CD101 positive TILs, at least 1×10⁶ TILs CD101 positive TILs, at least 1×10⁷ TILs CD101 positive TILs, at least 1×10⁸ TILs CD101 positive TILs. In some embodiments, the selection of CD101 positive TILs occurs until there are at least 1×10⁶ TILs CD101 positive TILs.

In some embodiments, the selection step, exemplified as Step A2 of FIG. 1, comprises the steps of (i) exposing the first population of TILs to an excess of a monoclonal anti-CD101 IgG4 antibody that binds to CD101 through an N-terminal loop outside the IgV domain of CD101, (ii) adding an excess of an anti-IgG4 antibody conjugated to a fluorophore, and (iii) performing a flow-based cell sort based on the fluorophore to obtain a CD101 enriched TIL population. In some embodiments, the monoclonal anti-CD101 IgG4 antibody is nivolumab or variants, fragments, or conjugates thereof. In some embodiments, the anti-IgG4 antibody is clone anti-human IgG4, Clone HP6023. In some embodiments, the anti-CD101 antibody for use in the selection in step (b) binds to the same epitope as EH12.2H7 or nivolumab.

To determine if TILs derived from a tumor sample are CD101high, one skilled in the art can utilize a reference value corresponding to the level of expression of CD101 in peripheral T cells obtained from a blood sample from one or more healthy human subjects. CD101 positive cells in the reference sample can be defined using fluorescence minus one controls and matching isotype controls. In some embodiments, the expression level of CD101 is measured in CD3+/CD101+ peripheral T cells from a healthy subject (e.g., the reference cells) is used to establish a threshold value or cut-off value of immunostaining intensity of CD101 in TILs obtained from a tumor. The threshold value can be defined as the minimal intensity of CD101 immunostaining of CD101high T cells. As such, TILs with a CD101 expression that is the same or above the threshold value can be considered to be CD101high cells. In some instances, the CD101high TILs represent those with the highest intensity of CD101 immunostaining corresponding to a maximum 1% or less of the total CD3+ cells. In other instances, the CD101high TILs represent those with the highest intensity of CD101 immunostaining corresponding to the maximum 0.75% or less of the total CD3+ cells. In some instances, the CD101high TILs represent those with the highest intensity of CD101 immunostaining corresponding to the maximum 0.50% or less of the total CD3+ cells. In one instance, the CD101high TILs represent those with the highest intensity of CD101 immunostaining corresponding to the maximum 0.25% or less of the total CD3+ cells.

In some embodiments, the PD-1 gating method of WO2019156568 is employed for CD101. To determine if TILs derived from a tumor sample are CD101high, one skilled in the art can utilize a reference value corresponding to the level of expression of CD101 in peripheral T cells obtained from a blood sample from one or more healthy human subjects. CD101 positive cells in the reference sample can be defined using fluorescence minus one controls and matching isotype controls. In some embodiments, the expression level of CD101 is measured in CD3+/CD101+ peripheral T cells from a healthy subject (e.g., the reference cells) is used to establish a threshold value or cut-off value of immunostaining intensity of CD101 in TILs obtained from a tumor. The threshold value can be defined as the minimal intensity of CD101 immunostaining of CD101high T cells. As such, TILs with a CD101 expression that is the same or above the threshold value can be considered to be CD101high cells. In some instances, the CD101high TILs represent those with the highest intensity of CD101 immunostaining corresponding to a maximum 1% or less of the total CD3+ cells. In other instances, the CD101high TILs represent those with the highest intensity of CD101 immunostaining corresponding to the maximum 0.75% or less of the total CD3+ cells. In some instances, the CD101high TILs represent those with the highest intensity of CD101 immunostaining corresponding to the maximum 0.50% or less of the total CD3+ cells. In one instance, the CD101high TILs represent those with the highest intensity of CD101 immunostaining corresponding to the maximum 0.25% or less of the total CD3+ cells.

In some embodiments, the CD101 positive (CD101+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-CD101 antibody linked to a fluorophore and an anti-CD3 antibody linked to a fluorophore. In some embodiments, the primary cell population TILs are stained with a cocktail that includes an anti-CD101 antibody linked to a fluorophore (for example, PE, live/dead violet) and anti-CD3-FITC. In some embodiments, the primary cell population TILs are stained with a cocktail that includes anti-CD101-PE, anti-CD3-FITC and live/dead blue stain (ThermoFisher, MA, Cat #L23105). In some embodiments, the after incubation with the anti-PD1 antibody, CD101 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

10. LAG3—Preselection Selection for LAG3 (as exemplified in Step A2 of FIG. 1)

According to the methods of the present invention, the TILs are preselected for being LAG3 positive (LAG3+) prior to the priming first expansion.

In some embodiments, the TILs of the present invention are preselected for an exhaustion marker such as LAG3 (Philip, M., et al., Nature. 545(7655):452-456 (2017)). According to the methods of the present invention, the TILs are preselected for being LAG3 positive (LAG3+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are LAG3 positive (LAG3+) (for example, after preselection and before the priming first expansion). In some embodiments, TILs for use in the priming first expansion are at least 75% LAG3 positive, at least 80% LAG3 positive, at least 85% LAG3positive, at least 90% LAG3 positive, at least 95% LAG3 positive, at least 98% LAG3positive or at least 99% LAG3positive (for example, after preselection and before the priming first expansion). In some embodiments, the LAG3 population is LAG3high. In some embodiments, TILs for use in the priming first expansion are at least 25% LAG3high, at least 30% LAG3high, at least 35% LAG3high, at least 40% LAG3high, at least 45% LAG3high, at least 50% LAG3high, at least 55% LAG3high, at least 60% LAG3high, at least 65% LAG3high, at least 70% LAG3high, at least 75% LAG3high, at least 80% LAG3high, at least 85% LAG3high, at least 90% LAG3high, at least 95% LAG3high, at least 98% LAG3high or at least 99% LAG3high (for example, after preselection and before the priming first expansion).

In some embodiments, LAG3high is indicated by a TIL population that is at least 75% LAG3 positive, at least 80% LAG3 positive, at least 85% LAG3 positive, at least 90% LAG3 positive, at least 95% LAG3 positive, at least 98% LAG3 positive or at least 99% LAG3 positive, or 100% LAG3 positive. In some embodiments, LAG3high is indicated by a TIL population that is at least 80% LAG3 positive. In some embodiments, LAG3high is indicated by a TIL population that is at least 85% LAG3 positive. In some embodiments, LAG3high is indicated by a TIL population that is at least 90% LAG3 positive. In some embodiments, LAG3high is indicated by a TIL population that is at least 95% LAG3 positive. In some embodiments, LAG3high is indicated by a TIL population that is at least 98% LAG3 positive. In some embodiments, LAG3high is indicated by a TIL population that is at least 99% LAG3 positive. In some embodiments, LAG3high is indicated by a TIL population that is 100% LAG3 positive.

In some embodiments, LAG3high is indicated by a TIL population wherein the TILs express at least 25% more LAG3 than a control or baseline LAG3 level, express at least 30% more LAG3 than a control or baseline LAG3 level, express at least 35% more LAG3 than a control or baseline LAG3 level, express at least 40% more LAG3 than a control or baseline LAG3 level, express at least 45% more LAG3 than a control or baseline LAG3 level, express at least 50% more LAG3 than a control or baseline LAG3 level, express at least 55% more LAG3 than a control or baseline LAG3 level, express at least 60% more LAG3 than a control or baseline LAG3 level, express at least 65% more LAG3 than a control or baseline LAG3 level, express at least 70% more LAG3 than a control or baseline LAG3 level, express at least 75% more LAG3 than a control or baseline LAG3 level, express at least 80% more LAG3 than a control or baseline LAG3 level, express at least 85% more LAG3 than a control or baseline LAG3 level, express at least 90% more LAG3 than a control or baseline LAG3 level, express at least 95% more LAG3 than a control or baseline LAG3 level, express at least 99% more LAG3 than a control or baseline LAG3 level, or express 100% more LAG3 than a control or baseline LAG3 level.

In some embodiments, LAG3high is indicated by a TIL population wherein the TILs express 1-fold or more LAG3 than a control or baseline LAG3 level. In some embodiments, LAG3high is indicated by a TIL population wherein the TILs express one-fold, two-fold, three-fold, four-fold, five-fold, ten-fold, or more LAG3 than a control or baseline LAG3 level. In some embodiments, LAG3high is indicated by a TIL population wherein the TILs express one-fold more LAG3 than a control or baseline LAG3 level. In some embodiments, LAG3high is indicated by a TIL population wherein the TILs express two-fold more LAG3 than a control or baseline LAG3 level. In some embodiments, LAG3high is indicated by a TIL population wherein the TILs express three-fold more LAG3 than a control or baseline LAG3 level. In some embodiments, LAG3high is indicated by a TIL population wherein the TILs express four-fold more LAG3 than a control or baseline LAG3 level. In some embodiments, LAG3high is indicated by a TIL population wherein the TILs express five-fold more LAG3 than a control or baseline LAG3 level. In some embodiments, LAG3high is indicated by a TIL population wherein the TILs express ten-fold more LAG3 than a control or baseline LAG3

In some embodiments, the preselection of LAG3 positive TILs is performed by staining primary cell population, whole tumor digests, and/or whole tumor cell suspensions TILs with an anti-LAG3 antibody. In some embodiments, the anti-LAG3 antibody is a polyclonal antibody e.g., a mouse anti-human LAG3 polyclonal antibody, a goat anti-human LAG3 polyclonal antibody, etc. In some embodiments, the anti-LAG3 antibody is a monoclonal antibody. In some embodiments the anti-LAG3 antibody includes, e.g., but is not limited to TSR-033, Sym022 (Anti-LAG-3), BMS 986016, GSK2831781, and/or LAG525.

In some embodiments, the anti-LAG3 antibody for use in the preselection binds at least 75%, at least 80%, at least 85%, at least 90%, at least 95%, at least 98%, at least 99% or at least 100% of the cells expressing LAG3.

In some embodiments, the patient has been treated with an anti-LAG3 antibody. In some embodiments, the subject is anti-LAG3 antibody treatment naïve. In some embodiments, the subject has not been treated with an anti-LAG3 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-LAG3 antibody treatment. In some embodiments, the subject is post-chemotherapeutic treatment and post anti-LAG3 antibody treatment. In some embodiments, the patient is anti-LAG3 antibody treatment naïve. In some embodiments, the subject has treatment naïve cancer or is post-chemotherapeutic treatment but anti-LAG3 antibody treatment naïve. In some embodiments, the subject is treatment naïve and post-chemotherapeutic treatment but anti-LAG3 antibody treatment naive.

In some embodiments in which the patient has been previously treated with a first anti-LAG3 antibody, the preselection is performed by staining the primary cell population, whole tumor digests, and/or whole tumor cell suspensions TILs with a second anti-LAG3 antibody that is not blocked by the first anti-LAG3 antibody from binding to LAG3 on the surface of the primary cell population TILs.

In some embodiments in which the patient has been previously treated with an anti-LAG3 antibody, the preselection is performed by staining the primary cell population TILs with an antibody (an “anti-Fc antibody”) that binds to the Fc region of the anti-LAG3 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-LAG3 human or humanized IgG antibody, and the primary cell population TILs are stained with an anti-human IgG antibody. In some embodiments in which the patient has been previously treated with an anti-LAG3 human or humanized IgG1 antibody, the primary cell population TILs are stained with an anti-human IgG1 antibody. In some embodiments in which the patient has been previously treated with an anti-LAG3 human or humanized IgG2 antibody, the primary cell population TILs are stained with an anti-human IgG2 antibody. In some embodiments in which the patient has been previously treated with an anti-LAG3 human or humanized IgG3 antibody, the primary cell population TILs are stained with an anti-human IgG3 antibody. In some embodiments in which the patient has been previously treated with an anti-LAG3 human or humanized IgG4 antibody, the primary cell population TILs are stained with an anti-human IgG4 antibody.

In some embodiments in which the patient has been previously treated with an anti-LAG3 antibody, the preselection is performed by contacting the primary cell population TILs with the same anti-LAG3 antibody and then staining the primary cell population TILs with an anti-Fc antibody that binds to the Fc region of the anti-LAG3 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to LAG3 negative TILs, LAG3 intermediate TILs, and LAG3 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the LAG3high population is defined as the population of cells that is positive for LAG3 above what is observed in PBMCs. In some embodiments, the intermediate LAG3+ population in the TIL is encompasses the LAG3+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days, 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the LAG3 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%±0.25%. In some embodiments, the gating and compensation for the mean fluorescence intensity (MFI) is in the range of about 1.75%±0.25%. In some embodiments, the gating and compensation for the mean fluorescence intensity (MFI) is in the range of about L75%±0.25% when setting the LAG3 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of LAG3 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the LAG3 high gate. In some embodiments, the MFI calculation is the mean value measured using at least two samples of PBMCs. In some embodiments, the MFI calculation is the median value measured using at least two samples of PBMCs.

In some embodiments, the LAG3 positive (LAG3+) cells are sorted by FACs and/or other flow cytometry method. In some embodiments, the LAG3 positive TILs are LAG3high TILs. In some embodiments, the LAG3 positive TILs are LAG3intermediate TILs. In some embodiments, the LAG3+ cells are sorted by employing a bead selection method. In some embodiments, the LAG3+ cells are sorted by employing a magnetic bead selection method. In some embodiments, the bead selection employs an antibody bound bead, for example but no limited to a commercially available bead, such as Miltenyi or Fisher, for selection. In some embodiments, the LAG3+ high cells are sorted by employing a bead selection method. In some embodiments, the LAG3+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-LAG3 antibody is conjugated to the bead, either directly or indirectly. In some embodiments, the bead selection process selects for both LAG3+ and CD3+ TILs. In some embodiments the anti-LAG3 antibody employed in the bead selection method includes, e.g., but is not limited to TSR-033, Sym022 (Anti-LAG-3), BMS 986016, GSK2831781, and/or LAG525.

In some embodiments, the collection buffer employed to collect the LAG3+ cells and/or the LAG3 negative cells does not include serum. In some embodiments, the collection buffer employed to collect the LAG3+ cells and/or the LAG3 negative cells includes serum. In some embodiments, the collection buffer employed to collect the LAG3+ cells and/or the LAG3 negative cells includes a component to mitigate or reduce viscosity differences between sort buffer and downstream buffers and/or media. In some embodiments, the collection buffer employed to collect the LAG3+ cells and/or the LAG3 negative cells includes only human serum albumin (HSA). In some embodiments, the collection buffer employed to collect the LAG3+ cells and/or the LAG3 negative cells includes an equal amount of HSA and PBS/EDTA Buffer. In some embodiments, the collection buffer employed to collect the LAG3+ cells and/or the LAG3 negative cells includes HSA and PBS/EDTA Buffer at a 1:1, L2, 1:3, 1:4, 2:1, 3:1, or 4:1 ratio.

In some embodiments, preselection involves selecting LAG3 positive TILs from the first population of TILs to obtain a LAG3 enriched TIL population comprises the selecting a population of TILs from a first population of TILs that are at least 11.27% to 74.4% LAG3 positive TILs. In some embodiments, the first population of TILs are at least 20% to 80% LAG3 positive TILs, at least 20% to 80% LAG3 positive TILs, at least 30% to 80% LAG3 positive TILs, at least 40% to 80% LAG3 positive TILs, at least 50% to 80% LAG3 positive TILs, at least 10% to 70% LAG3 positive TILs, at least 20% to 70% LAG3 positive TILs, at least 30% to 70% LAG3 positive TILs, or at least 40% to 70% LAG3 positive TILs.

In some embodiments, the selection step (e.g., preselection and/or selecting LAG3 positive cells) comprises the steps of:
(i) exposing the first population of TILs and a population of PBMC to an excess of a monoclonal anti-LAG3 IgG4 antibody that binds to LAG3 through an N-terminal loop outside the IgV domain of LAG3,
(ii) adding an excess of an anti-IgG4 antibody conjugated to a fluorophore,
(iii) obtaining the LAG3 enriched TIL population based on the intensity of the fluorophore of the LAG3 positive TILs in the first population of TILs compared to the intensity in the population of PBMCs as performed by fluorescence-activated cell sorting (FACS).

In some embodiments, the LAG3 positive TILs are LAG3high TILs.

In some embodiments, at least 70% of the LAG3 enriched TIL population are LAG3 positive TILs. In some embodiments, at least 80% of the LAG3 enriched TIL population are LAG3 positive TILs. In some embodiments, at least 90% of the LAG3 enriched TIL population are LAG3 positive TILs. In some embodiments, at least 95% of the LAG3 enriched TIL population are LAG3 positive TILs. In some embodiments, at least 99% of the LAG3 enriched TIL population are LAG3 positive TILs. In some embodiments, 100% of the LAG3 enriched TIL population are LAG3 positive TILs.

In some embodiments, the selection of LAG3 positive TILs occurs until there are at least 1×10⁴ TILs LAG3 positive TILs, at least 1×10⁵ TILs LAG3 positive TILs, at least 1×10⁶ TILs LAG3 positive TILs, at least 1×10⁷ TILs LAG3 positive TILs, at least 1×10⁸ TILs LAG3 positive TILs. In some embodiments, the selection of LAG3 positive TILs occurs until there are at least 1×10⁶ TILs LAG3 positive TILs.

In some embodiments, the selection step, exemplified as Step A2 of FIG. 1, comprises the steps of (i) exposing the first population of TILs to an excess of a monoclonal anti-LAG3 IgG4 antibody that binds to LAG3 through an N-terminal loop outside the IgV domain of LAG3, (ii) adding an excess of an anti-IgG4 antibody conjugated to a fluorophore, and (iii) performing a flow-based cell sort based on the fluorophore to obtain a LAG3 enriched TIL population. In some embodiments, the monoclonal anti-LAG3 IgG4 antibody is nivolumab or variants, fragments, or conjugates thereof. In some embodiments, the anti-IgG4 antibody is clone anti-human IgG4, Clone HP6023. In some embodiments, the anti-LAG3 antibody for use in the selection in step (b) binds to the same epitope as EH12.2H7 or nivolumab.

To determine if TILs derived from a tumor sample are LAG3high, one skilled in the art can utilize a reference value corresponding to the level of expression of LAG3 in peripheral T cells obtained from a blood sample from one or more healthy human subjects. LAG3 positive cells in the reference sample can be defined using fluorescence minus one controls and matching isotype controls. In some embodiments, the expression level of LAG3 is measured in CD3+/LAG3+ peripheral T cells from a healthy subject (e.g., the reference cells) is used to establish a threshold value or cut-off value of immunostaining intensity of LAG3 in TILs obtained from a tumor. The threshold value can be defined as the minimal intensity of LAG3 immunostaining of LAG3high T cells. As such, TILs with a LAG3 expression that is the same or above the threshold value can be considered to be LAG3high cells. In some instances, the LAG3high TILs represent those with the highest intensity of LAG3 immunostaining corresponding to a maximum 1% or less of the total CD3+ cells. In other instances, the LAG3high TILs represent those with the highest intensity of LAG3 immunostaining corresponding to the maximum 0.75% or less of the total CD3+ cells. In some instances, the LAG3high TILs represent those with the highest intensity of LAG3 immunostaining corresponding to the maximum 0.50% or less of the total CD3+ cells. In one instance, the LAG3high TILs represent those with the highest intensity of LAG3 immunostaining corresponding to the maximum 0.25% or less of the total CD3+ cells.

In some embodiments, the PD-1 gating method of WO2019156568 is employed for LAG3. To determine if TILs derived from a tumor sample are LAG3high, one skilled in the art can utilize a reference value corresponding to the level of expression of LAG3 in peripheral T cells obtained from a blood sample from one or more healthy human subjects. LAG3 positive cells in the reference sample can be defined using fluorescence minus one controls and matching isotype controls. In some embodiments, the expression level of LAG3 is measured in CD3+/LAG3+ peripheral T cells from a healthy subject (e.g., the reference cells) is used to establish a threshold value or cut-off value of immunostaining intensity of LAG3 in TILs obtained from a tumor. The threshold value can be defined as the minimal intensity of LAG3 immunostaining of LAG3high T cells. As such, TILs with a LAG3 expression that is the same or above the threshold value can be considered to be LAG3high cells. In some instances, the LAG3high TILs represent those with the highest intensity of LAG3 immunostaining corresponding to a maximum 1% or less of the total CD3+ cells. In other instances, the LAG3high TILs represent those with the highest intensity of LAG3 immunostaining corresponding to the maximum 0.75% or less of the total CD3+ cells. In some instances, the LAG3high TILs represent those with the highest intensity of LAG3 immunostaining corresponding to the maximum 0.50% or less of the total CD3+ cells. In one instance, the LAG3high TILs represent those with the highest intensity of LAG3 immunostaining corresponding to the maximum 0.25% or less of the total CD3+ cells.

In some embodiments, the LAG3 positive (LAG3+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-LAG3 antibody linked to a fluorophore and an anti-CD3 antibody linked to a fluorophore. In some embodiments, the primary cell population TILs are stained with a cocktail that includes an anti-LAG3 antibody linked to a fluorophore (for example, PE, live/dead violet) and anti-CD3-FITC. In some embodiments, the primary cell population TILs are stained with a cocktail that includes anti-LAG3-PE, anti-CD3-FITC and live/dead blue stain (ThermoFisher, MA, Cat #L23105). In some embodiments, the after incubation with the anti-PD1 antibody, LAG3 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

11. TIM3—Preselection Selection for TIM3 (as exemplified in Step A2 of FIG. 1)

According to the methods of the present invention, the TILs are preselected for being TIM3 positive (TIM3+) prior to the priming first expansion.

In some embodiments, the TILs of the present invention are preselected for an exhaustion marker such as TIM3. According to the methods of the present invention, the TILs are preselected for being TIM3 positive (TIM3+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are TIM3 positive (TIM3+) (for example, after preselection and before the priming first expansion). In some embodiments, TILs for use in the priming first expansion are at least 75% TIM3 positive, at least 80% TIM3 positive, at least 85% TIM3positive, at least 90% TIM3 positive, at least 95% TIM3 positive, at least 98% TIM3positive or at least 99% TIM3positive (for example, after preselection and before the priming first expansion). In some embodiments, the TIM3 population is TIM3high. In some embodiments, TILs for use in the priming first expansion are at least 25% TIM3high, at least 30% TIM3high, at least 35% TIM3high, at least 40% TIM3high, at least 45% TIM3high, at least 50% TIM3high, at least 55% TIM3high, at least 60% TIM3high, at least 65% TIM3high, at least 70% TIM3high, at least 75% TIM3high, at least 80% TIM3high, at least 85% TIM3high, at least 90% TIM3high, at least 95% TIM3high, at least 98% TIM3high or at least 99% TIM3high (for example, after preselection and before the priming first expansion).

In some embodiments, TIM3high is indicated by a TIL population that is at least 75% TIM3 positive, at least 80% TIM3 positive, at least 85% TIM3 positive, at least 90% TIM3 positive, at least 95% TIM3 positive, at least 98% TIM3 positive or at least 99% TIM3 positive, or 100% TIM3 positive. In some embodiments, TIM3high is indicated by a TIL population that is at least 80% TIM3 positive. In some embodiments, TIM3high is indicated by a TIL population that is at least 85% TIM3 positive. In some embodiments, TIM3high is indicated by a TIL population that is at least 90% TIM3 positive. In some embodiments, TIM3high is indicated by a TIL population that is at least 95% TIM3 positive. In some embodiments, TIM3high is indicated by a TIL population that is at least 98% TIM3 positive. In some embodiments, TIM3high is indicated by a TIL population that is at least 99% TIM3 positive. In some embodiments, TIM3high is indicated by a TIL population that is 100% TIM3 positive.

In some embodiments, TIM3high is indicated by a TIL population wherein the TILs express at least 25% more TIM3 than a control or baseline TIM3 level, express at least 30% more TIM3 than a control or baseline TIM3 level, express at least 35% more TIM3 than a control or baseline TIM3 level, express at least 40% more TIM3 than a control or baseline TIM3 level, express at least 45% more TIM3 than a control or baseline TIM3 level, express at least 50% more TIM3 than a control or baseline TIM3 level, express at least 55% more TIM3 than a control or baseline TIM3 level, express at least 60% more TIM3 than a control or baseline TIM3 level, express at least 65% more TIM3 than a control or baseline TIM3 level, express at least 70% more TIM3 than a control or baseline TIM3 level, express at least 75% more TIM3 than a control or baseline TIM3 level, express at least 80% more TIM3 than a control or baseline TIM3 level, express at least 85% more TIM3 than a control or baseline TIM3 level, express at least 90% more TIM3 than a control or baseline TIM3 level, express at least 95% more TIM3 than a control or baseline TIM3 level, express at least 99% more TIM3 than a control or baseline TIM3 level, or express 100% more TIM3 than a control or baseline TIM3 level.

In some embodiments, TIM3high is indicated by a TIL population wherein the TILs express 1-fold or more TIM3 than a control or baseline TIM3 level. In some embodiments, TIM3high is indicated by a TIL population wherein the TILs express one-fold, two-fold, three-fold, four-fold, five-fold, ten-fold, or more TIM3 than a control or baseline TIM3 level. In some embodiments, TIM3high is indicated by a TIL population wherein the TILs express one-fold more TIM3 than a control or baseline TIM3 level. In some embodiments, TIM3high is indicated by a TIL population wherein the TILs express two-fold more TIM3 than a control or baseline TIM3 level. In some embodiments, TIM3high is indicated by a TIL population wherein the TILs express three-fold more TIM3 than a control or baseline TIM3 level. In some embodiments, TIM3high is indicated by a TIL population wherein the TILs express four-fold more TIM3 than a control or baseline TIM3 level. In some embodiments, TIM3high is indicated by a TIL population wherein the TILs express five-fold more TIM3 than a control or baseline TIM3 level. In some embodiments, TIM3high is indicated by a TIL population wherein the TILs express ten-fold more TIM3 than a control or baseline TIM3 level.

In some embodiments, the preselection of TIM3 positive TILs is performed by staining primary cell population, whole tumor digests, and/or whole tumor cell suspensions TILs with an anti-TIM3 antibody. In some embodiments, the anti-TIM3 antibody is a polyclonal antibody e.g., a mouse anti-human TIM3 polyclonal antibody, a goat anti-human TIM3 polyclonal antibody, etc. In some embodiments, the anti-TIM3 antibody is a monoclonal antibody. In some embodiments the anti-TIM3 antibody includes, e.g., but is not limited to MAB2365, PA1-41295, ab185703, TSR-022, LY3321367, BGB-A425, Sym023, MBG453, and/or INCAGN02390.

In some embodiments, the anti-TIM3 antibody for use in the preselection binds at least 75%, at least 80%, at least 85%, at least 90%, at least 95%, at least 98%, at least 99% or at least 100% of the cells expressing TIM3.

In some embodiments, the patient has been treated with an anti-TIM3 antibody. In some embodiments, the subject is anti-TIM3 antibody treatment naïve. In some embodiments, the subject has not been treated with an anti-TIM3 antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-TIM3 antibody treatment. In some embodiments, the subject is post-chemotherapeutic treatment and post anti-TIM3 antibody treatment. In some embodiments, the patient is anti-TIM3 antibody treatment naïve. In some embodiments, the subject has treatment naïve cancer or is post-chemotherapeutic treatment but anti-TIM3 antibody treatment naïve. In some embodiments, the subject is treatment naïve and post-chemotherapeutic treatment but anti-TIM3 antibody treatment naive.

In some embodiments in which the patient has been previously treated with a first anti-TIM3 antibody, the preselection is performed by staining the primary cell population, whole tumor digests, and/or whole tumor cell suspensions TILs with a second anti-TIM3 antibody that is not blocked by the first anti-TIM3 antibody from binding to TIM3 on the surface of the primary cell population TILs.

In some embodiments in which the patient has been previously treated with an anti-TIM3 antibody, the preselection is performed by staining the primary cell population TILs with an antibody (an “anti-Fc antibody”) that binds to the Fc region of the anti-TIM3 antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-TIM3 human or humanized IgG antibody, and the primary cell population TILs are stained with an anti-human IgG antibody. In some embodiments in which the patient has been previously treated with an anti-TIM3 human or humanized IgG1 antibody, the primary cell population TILs are stained with an anti-human IgG1 antibody. In some embodiments in which the patient has been previously treated with an anti-TIM3 human or humanized IgG2 antibody, the primary cell population TILs are stained with an anti-human IgG2 antibody. In some embodiments in which the patient has been previously treated with an anti-TIM3 human or humanized IgG3 antibody, the primary cell population TILs are stained with an anti-human IgG3 antibody. In some embodiments in which the patient has been previously treated with an anti-TIM3 human or humanized IgG4 antibody, the primary cell population TILs are stained with an anti-human IgG4 antibody.

In some embodiments in which the patient has been previously treated with an anti-TIM3 antibody, the preselection is performed by contacting the primary cell population TILs with the same anti-TIM3 antibody and then staining the primary cell population TILs with an anti-Fc antibody that binds to the Fc region of the anti-TIM3 antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to TIM3 negative TILs, TIM3 intermediate TILs, and TIM3 positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the TIM3high population is defined as the population of cells that is positive for TIM3 above what is observed in PBMCs. In some embodiments, the intermediate TIM3+ population in the TIL is encompasses the TIM3+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days. 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the TIM3 pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%±0.25%. In some embodiments, the gating and compensation for the mean fluorescence intensity (MFI) is in the range of about 1.75%±0.25%. In some embodiments, the gating and compensation for the mean fluorescence intensity (MFI) is in the range of about 1.75%±0.25% when setting the TIM3 high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of TIM3 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the TIM3 high gate. In some embodiments, the MFI calculation is the mean value measured using at least two samples of PBMCs. In some embodiments, the MFI calculation is the median value measured using at least two samples of PBMCs.

In some embodiments, the TIM3 positive (TIM3+) cells are sorted by FACs and/or other flow cytometry method. In some embodiments, the TIM3 positive TILs are TIM3high TILs. In some embodiments, the TIM3 positive TILs are TIM3intermediate TILs. In some embodiments, the TIM3+ cells are sorted by employing a bead selection method. In some embodiments, the TIM3+ cells are sorted by employing a magnetic bead selection method. In some embodiments, the bead selection employs an antibody bound bead, for example but no limited to a commercially available bead, such as Miltenyi or Fisher, for selection. In some embodiments, the TIM3+ high cells are sorted by employing a bead selection method. In some embodiments, the TIM3+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-TIM3 antibody is conjugated to the bead, either directly or indirectly. In some embodiments, the bead selection process selects for both TIM3+ and CD3+ TILs. In some embodiments the anti-TIM3 antibody includes, e.g., but is not limited to MAB2365, PA1-41295, ab185703, TSR-022, LY3321367, BGB-A425, Sym023, MBG453, and/or INCAGN02390.

In some embodiments, the collection buffer employed to collect the TIM3+ cells and/or the TIM3 negative cells does not include serum. In some embodiments, the collection buffer employed to collect the TIM3+ cells and/or the TIM3 negative cells includes serum. In some embodiments, the collection buffer employed to collect the TIM3+ cells and/or the TIM3 negative cells includes a component to mitigate or reduce viscosity differences between sort buffer and downstream buffers and/or media. In some embodiments, the collection buffer employed to collect the TIM3+ cells and/or the TIM3 negative cells includes only human serum albumin (HSA). In some embodiments, the collection buffer employed to collect the TIM3+ cells and/or the TIM3 negative cells includes an equal amount of HSA and PBS/EDTA Buffer. In some embodiments, the collection buffer employed to collect the TIM3+ cells and/or the TIM3 negative cells includes HSA and PBS/EDTA Buffer at a 1:1, 1:2, 1:3, 1:4, 2:1, 3:1, or 4:1 ratio.

In some embodiments, preselection involves selecting TIM3 positive TILs from the first population of TILs to obtain a TIM3 enriched TIL population comprises the selecting a population of TILs from a first population of TILs that are at least 11.27% to 74.4% TIM3 positive TILs. In some embodiments, the first population of TILs are at least 20% to 80% TIM3 positive TILs, at least 20% to 80% TIM3 positive TILs, at least 30% to 80% TIM3 positive TILs, at least 40% to 80% TIM3 positive TILs, at least 50% to 80% TIM3 positive TILs, at least 10% to 70% TIM3 positive TILs, at least 20% to 70% TIM3 positive TILs, at least 30% to 70% TIM3 positive TILs, or at least 40% to 70% TIM3 positive TILs.

In some embodiments, the selection step (e.g., preselection and/or selecting TIM3 positive cells) comprises the steps of:
(i) exposing the first population of TILs and a population of PBMC to an excess of a monoclonal anti-TIM3 IgG4 antibody that binds to TIM3 through an N-terminal loop outside the IgV domain of TIM3,
(ii) adding an excess of an anti-IgG4 antibody conjugated to a fluorophore,
(iii) obtaining the TIM3 enriched TIL population based on the intensity of the fluorophore of the TIM3 positive TILs in the first population of TILs compared to the intensity in the population of PBMCs as performed by fluorescence-activated cell sorting (FACS).

In some embodiments, the TIM3 positive TILs are TIM3high TILs.

In some embodiments, at least 70% of the TIM3 enriched TIL population are TIM3 positive TILs. In some embodiments, at least 80% of the TIM3 enriched TIL population are TIM3 positive TILs. In some embodiments, at least 90% of the TIM3 enriched TIL population are TIM3 positive TILs. In some embodiments, at least 95% of the TIM3 enriched TIL population are TIM3 positive TILs. In some embodiments, at least 99% of the TIM3 enriched TIL population are TIM3 positive TILs. In some embodiments, 100% of the TIM3 enriched TIL population are TIM3 positive TILs.

In some embodiments, the selection of TIM3 positive TILs occurs until there are at least 1×10⁴ TILs TIM3 positive TILs, at least 1×10⁵ TILs TIM3 positive TILs, at least 1×10⁶ TILs TIM3 positive TILs, at least 1×10⁷ TILs TIM3 positive TILs, at least 1×10⁸ TILs TIM3 positive TILs. In some embodiments, the selection of TIM3 positive TILs occurs until there are at least 1×10⁶ TILs TIM3 positive TILs.

In some embodiments, the selection step, exemplified as Step A2 of FIG. 1, comprises the steps of (i) exposing the first population of TILs to an excess of a monoclonal anti-TIM3 IgG4 antibody that binds to TIM3 through an N-terminal loop outside the IgV domain of TIM3. (ii) adding an excess of an anti-IgG4 antibody conjugated to a fluorophore, and (iii) performing a flow-based cell sort based on the fluorophore to obtain a TIM3 enriched TIL population. In some embodiments, the monoclonal anti-TIM3 IgG4 antibody is nivolumab or variants, fragments, or conjugates thereof. In some embodiments, the anti-IgG4 antibody is clone anti-human IgG4, Clone HP6023. In some embodiments, the anti-TIM3 antibody for use in the selection in step (b) binds to the same epitope as EH12.2H7 or nivolumab.

To determine if TILs derived from a tumor sample are TIM3high, one skilled in the art can utilize a reference value corresponding to the level of expression of TIM3 in peripheral T cells obtained from a blood sample from one or more healthy human subjects. TIM3 positive cells in the reference sample can be defined using fluorescence minus one controls and matching isotype controls. In some embodiments, the expression level of TIM3 is measured in CD3+/TIM3+ peripheral T cells from a healthy subject (e.g., the reference cells) is used to establish a threshold value or cut-off value of immunostaining intensity of TIM3 in TILs obtained from a tumor. The threshold value can be defined as the minimal intensity of TIM3 immunostaining of TIM3high T cells. As such, TILs with a TIM3 expression that is the same or above the threshold value can be considered to be TIM3high cells. In some instances, the TIM3high TILs represent those with the highest intensity of TIM3 immunostaining corresponding to a maximum 1% or less of the total CD3+ cells. In other instances, the TIM3high TILs represent those with the highest intensity of TIM3 immunostaining corresponding to the maximum 0.75% or less of the total CD3+ cells. In some instances, the TIM3high TILs represent those with the highest intensity of TIM3 immunostaining corresponding to the maximum 0.50% or less of the total CD3+ cells. In one instance, the TIM3high TILs represent those with the highest intensity of TIM3 immunostaining corresponding to the maximum 0.25% or less of the total CD3+ cells.

In some embodiments, the PD-1 gating method of WO2019156568 is employed for TIM3. To determine if TILs derived from a tumor sample are TIM3high, one skilled in the art can utilize a reference value corresponding to the level of expression of TIM3 in peripheral T cells obtained from a blood sample from one or more healthy human subjects. TIM3 positive cells in the reference sample can be defined using fluorescence minus one controls and matching isotype controls. In some embodiments, the expression level of TIM3 is measured in CD3+/TIM3+ peripheral T cells from a healthy subject (e.g., the reference cells) is used to establish a threshold value or cut-off value of immunostaining intensity of TIM3 in TILs obtained from a tumor. The threshold value can be defined as the minimal intensity of TIM3 immunostaining of TIM3high T cells. As such, TILs with a TIM3 expression that is the same or above the threshold value can be considered to be TIM3high cells. In some instances, the TIM3high TILs represent those with the highest intensity of TIM3 immunostaining corresponding to a maximum 1% or less of the total CD3+ cells. In other instances, the TIM3high TILs represent those with the highest intensity of TIM3 immunostaining corresponding to the maximum 0.75% or less of the total CD3+ cells. In some instances, the TIM3high TILs represent those with the highest intensity of TIM3 immunostaining corresponding to the maximum 0.50% or less of the total CD3+ cells. In one instance, the TIM3high TILs represent those with the highest intensity of TIM3 immunostaining corresponding to the maximum 0.25% or less of the total CD3+ cells.

In some embodiments, the TIM3 positive (TIM3+) cells selected can be frozen prior to proceeding with the priming first expansion, for example, Step B of FIG. 1 (in particular, e.g., FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-TIM3 antibody linked to a fluorophore and an anti-CD3 antibody linked to a fluorophore. In some embodiments, the primary cell population TILs are stained with a cocktail that includes an anti-TIM3 antibody linked to a fluorophore (for example, PE, live/dead violet) and anti-CD3-FITC. In some embodiments, the primary cell population TILs are stained with a cocktail that includes anti-TIM3-PE, anti-CD3-FITC and live/dead blue stain (ThermoFisher, MA, Cat #L23105). In some embodiments, the after incubation with the anti-PD1 antibody, TIM3 positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine; rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

12. TIGIT—Preselection Selection for TIGIT (as exemplified in Step A2 of FIG. 1)

According to the methods of the present invention, the TILs are preselected for being TIGIT positive (TIGIT+) prior to the priming first expansion.

In some embodiments, the TILs of the present invention are preselected for an exhaustion marker such as TIGIT (Philip, M., et al., Nature. 545(7655):452-456 (2017)). According to the methods of the present invention, the TILs are preselected for being TIGIT positive (TIGIT+) prior to the priming first expansion.

In some embodiments, a minimum of 3,000 TILs are needed for seeding into the first expansion. In some embodiments, the preselection step yields a minimum of 3,000 TILs. In some embodiments, a minimum of 4,000 TILs are needed for seeding into the first expansion. In some embodiments, the preselection step yields a minimum of 4,000 TILs. In some embodiments, a minimum of 5,000 TILs are needed for seeding into the first expansion. In some embodiments, the preselection step yields a minimum of 5,000 TILs. In some embodiments, a minimum of 6,000 TILs are needed for seeding into the first expansion. In some embodiments, the preselection step yields a minimum of 6,000 TILs. In some embodiments, a minimum of 7,000 TILs are needed for seeding into the first expansion. In some embodiments, the preselection step yields a minimum of 7,000 TILs. In some embodiments, a minimum of 8,000 TILs are needed for seeding into the first expansion. In some embodiments, the preselection step yields a minimum of 8,000 TILs. In some embodiments, a minimum of 9,000 TILs are needed for seeding into the first expansion. In some embodiments, the preselection step yields a minimum of 9,000 TILs. In some embodiments, a minimum of 10,000 TILs are needed for seeding into the first expansion. In some embodiments, the preselection step yields a minimum of 10,000 TILs. In some embodiments, cells are grown or expanded to a density of 200,000. In some embodiments, cells are grown or expanded to a density of 200,000 to provide about 2e8 TILs for initiating rapid second expansion. In some embodiments, cells are grown or expanded to a density of 150,000. In some embodiments, cells are grown or expanded to a density of 150,000 to provide about 2e8 TILs for initiating rapid second expansion. In some embodiments, cells are grown or expanded to a density of 250,000. In some embodiments, cells are grown or expanded to a density of 250,000 to provide about 2e8 TILs for initiating rapid second expansion. In some embodiments, the minimum cell density is 10,000 cells to give 10e6 for initiating rapid second expansion. In some embodiments, a 10e6 seeding density for initiating the rapid second expansion could yield greater than 1e9 TILs.

In some embodiments the TILs for use in the priming first expansion are TIGIT positive (TIGIT+) (for example, after preselection and before the priming first expansion). In some embodiments, TILs for use in the priming first expansion are at least 75% TIGIT positive, at least 80% TIGIT positive, at least 85% TIGITpositive, at least 90% TIGIT positive, at least 95% TIGIT positive, at least 98% TIGITpositive or at least 99% TIGITpositive (for example, after preselection and before the priming first expansion). In some embodiments, the TIGIT population is TIGIThigh. In some embodiments, TILs for use in the priming first expansion are at least 25% TIGIThigh, at least 30% TIGIThigh, at least 35% TIGIThigh, at least 40% TIGIThigh, at least 45% TIGIThigh, at least 50% TIGIThigh, at least 55% TIGIThigh, at least 60% TIGIThigh, at least 65% TIGIThigh, at least 70% TIGIThigh, at least 75% TIGIThigh, at least 80% TIGIThigh, at least 85% TIGIThigh, at least 90% TIGIThigh, at least 95% TIGIThigh, at least 98% TIGIThigh or at least 99% TIGIThigh (for example, after preselection and before the priming first expansion).

In some embodiments, TIGIThigh is indicated by a TIL population that is at least 75% TIGIT positive, at least 80% TIGIT positive, at least 85% TIGIT positive, at least 90% TIGIT positive, at least 95% TIGIT positive, at least 98% TIGIT positive or at least 99% TIGIT positive, or 100% TIGIT positive. In some embodiments, TIGIThigh is indicated by a TIL population that is at least 80% TIGIT positive. In some embodiments, TIGIThigh is indicated by a TIL population that is at least 85% TIGIT positive. In some embodiments, TIGIThigh is indicated by a TIL population that is at least 90% TIGIT positive. In some embodiments, TIGIThigh is indicated by a TIL population that is at least 95% TIGIT positive. In some embodiments, TIGIThigh is indicated by a TIL population that is at least 98% TIGIT positive. In some embodiments, TIGIThigh is indicated by a TIL population that is at least 99% TIGIT positive. In some embodiments, TIGIThigh is indicated by a TIL population that is 100% TIGIT positive.

In some embodiments, TIGIThigh is indicated by a TIL population wherein the TILs express at least 25% more TIGIT than a control or baseline TIGIT level, express at least 30% more TIGIT than a control or baseline TIGIT level, express at least 35% more TIGIT than a control or baseline TIGIT level, express at least 40% more TIGIT than a control or baseline TIGIT level, express at least 45% more TIGIT than a control or baseline TIGIT level, express at least 50% more TIGIT than a control or baseline TIGIT level, express at least 55% more TIGIT than a control or baseline TIGIT level, express at least 60% more TIGIT than a control or baseline TIGIT level, express at least 65% more TIGIT than a control or baseline TIGIT level, express at least 70% more TIGIT than a control or baseline TIGIT level, express at least 75% more TIGIT than a control or baseline TIGIT level, express at least 80% more TIGIT than a control or baseline TIGIT level, express at least 85% more TIGIT than a control or baseline TIGIT level, express at least 90% more TIGIT than a control or baseline TIGIT level, express at least 95% more TIGIT than a control or baseline TIGIT level, express at least 99% more TIGIT than a control or baseline TIGIT level, or express 100% more TIGIT than a control or baseline TIGIT level.

In some embodiments, TIGIThigh is indicated by a TIL population wherein the TILs express 1-fold or more TIGIT than a control or baseline TIGIT level. In some embodiments, TIGIThigh is indicated by a TIL population wherein the TILs express one-fold, two-fold, three-fold, four-fold, five-fold, ten-fold, or more TIGIT than a control or baseline TIGIT level. In some embodiments, TIGIThigh is indicated by a TIL population wherein the TILs express one-fold more TIGIT than a control or baseline TIGIT level. In some embodiments, TIGIThigh is indicated by a TIL population wherein the TILs express two-fold more TIGIT than a control or baseline TIGIT level. In some embodiments, TIGIThigh is indicated by a TIL population wherein the TILs express three-fold more TIGIT than a control or baseline TIGIT level. In some embodiments, TIGIThigh is indicated by a TIL population wherein the TILs express four-fold more TIGIT than a control or baseline TIGIT level. In some embodiments, TIGIThigh is indicated by a TIL population wherein the TILs express five-fold more TIGIT than a control or baseline TIGIT level. In some embodiments, TIGIThigh is indicated by a TIL population wherein the TILs express ten-fold more TIGIT than a control or baseline TIGIT level.

In some embodiments, the preselection of TIGIT positive TILs is performed by staining primary cell population, whole tumor digests, and/or whole tumor cell suspensions TILs with an anti-TIGIT antibody. In some embodiments, the anti-TIGIT antibody is a polyclonal antibody e.g., a mouse anti-human TIGIT polyclonal antibody, a goat anti-human TIGIT polyclonal antibody, etc. In some embodiments, the anti-TIGIT antibody is a monoclonal antibody. In some embodiments the anti-TIGIT antibody includes, e.g., but is not limited to tiragolumab (anti-TIGIT, RG6058), BMS-986207, OMP-313M32, BGB-A1217, IBI939, COM902, EOS884448 (EOS-448), etigilimab, MK-7684, and/or AB154.

In some embodiments, the anti-TIGIT antibody for use in the preselection binds at least 75%, at least 80%, at least 85%, at least 90%, at least 95%, at least 98%, at least 99% or at least 100% of the cells expressing TIGIT.

In some embodiments, the patient has been treated with an anti-TIGIT antibody. In some embodiments, the subject is anti-TIGIT antibody treatment naïve. In some embodiments, the subject has not been treated with an anti-TIGIT antibody. In some embodiments, the subject has been previously treated with a chemotherapeutic agent. In some embodiments, the subject has been previously treated with a chemotherapeutic agent but is no longer being treated with the chemotherapeutic agent. In some embodiments, the subject is post-chemotherapeutic treatment or post anti-TIGIT antibody treatment. In some embodiments, the subject is post-chemotherapeutic treatment and post anti-TIGIT antibody treatment. In some embodiments, the patient is anti-TIGIT antibody treatment naïve. In some embodiments, the subject has treatment naïve cancer or is post-chemotherapeutic treatment but anti-TIGIT antibody treatment naïve. In some embodiments, the subject is treatment naïve and post-chemotherapeutic treatment but anti-TIGIT antibody treatment naive.

In some embodiments in which the patient has been previously treated with a first anti-TIGIT antibody, the preselection is performed by staining the primary cell population, whole tumor digests, and/or whole tumor cell suspensions TILs with a second anti-TIGIT antibody that is not blocked by the first anti-TIGIT antibody from binding to TIGIT on the surface of the primary cell population TILs.

In some embodiments in which the patient has been previously treated with an anti-TIGIT antibody, the preselection is performed by staining the primary cell population TILs with an antibody (an “anti-Fc antibody”) that binds to the Fc region of the anti-TIGIT antibody insolubilized on the surface of the primary cell population TILs. In some embodiments, the anti-Fc antibody is a polyclonal antibody e.g. mouse anti-human Fc polyclonal antibody, goat anti-human Fc polyclonal antibody, etc. In some embodiments, the anti-Fc antibody is a monoclonal antibody. In some embodiments in which the patient has been previously treated with an anti-TIGIT human or humanized IgG antibody, and the primary cell population TILs are stained with an anti-human IgG antibody. In some embodiments in which the patient has been previously treated with an anti-TIGIT human or humanized IgG1 antibody, the primary cell population TILs are stained with an anti-human IgG1 antibody. In some embodiments in which the patient has been previously treated with an anti-TIGIT human or humanized IgG2 antibody, the primary cell population TILs are stained with an anti-human IgG2 antibody. In some embodiments in which the patient has been previously treated with an anti-TIGIT human or humanized IgG3 antibody, the primary cell population TILs are stained with an anti-human IgG3 antibody. In some embodiments in which the patient has been previously treated with an anti-TIGIT human or humanized IgG4 antibody, the primary cell population TILs are stained with an anti-human IgG4 antibody.

In some embodiments in which the patient has been previously treated with an anti-TIGIT antibody, the preselection is performed by contacting the primary cell population TILs with the same anti-TIGIT antibody and then staining the primary cell population TILs with an anti-Fc antibody that binds to the Fc region of the anti-TIGIT antibody insolubilized on the surface of the primary cell population TILs.

In some embodiments, preselection is performed using a cell sorting method. In some embodiments, the cell sorting method is a flow cytometry method, e.g., flow activated cell sorting (FACS). In some embodiments, the intensity of the fluorophore in both the first population and the population of PBMCs is used to set up FACS gates for establishing low, medium, and high levels of intensity that correspond to TIGIT negative TILs, TIGIT intermediate TILs, and TIGIT positive TILs, respectively. In some embodiments, the cell sorting method is performed such that the gates are set at high, medium (also referred to as intermediate), and low (also referred to as negative) using the PBMC, the FMO control, and the sample itself to distinguish the three populations. In some embodiments, the PBMC is used as the gating control. In some embodiments, the TIGIThigh population is defined as the population of cells that is positive for TIGIT above what is observed in PBMCs. In some embodiments, the intermediate TIGIT+ population in the TIL is encompasses the TIGIT+ cells in the PBMC. In some embodiments, the negatives are gated based upon the FMO. In some embodiments, the FACS gates are set-up after the step of obtaining and/or receiving a first population of TILs from a tumor resected from a subject by processing a tumor sample obtained from the subject into multiple tumor fragments. In some embodiments, the gating is set up each sort. In some embodiments, the gating is set-up for each sample of PBMCs. In some embodiments, the gating is set-up for each sample of PBMCs. In some embodiments, the gating template is set-up from PBMC's every 10 days. 20 days, 30 days, 40 days, 50 days, or 60 days. In some embodiments, the gating template is set-up from PBMC's every 60 days. In some embodiments, the gating template is set-up for each sample of PBMC's every 10 days, 20 days, 30 days, 40 days, 50 days, or 60 days. In some embodiments, the gating template is set-up for each sample of PBMC's every 60 days.

In some embodiments, the gating for the TIGIT pre-selection is fixed for each pre-selection procedure. In some embodiments, the gating procedure that is fixed is a CD3+ gating procedure. In some embodiments, the gating procedure is not fixed but is determined based one the population obtained during each sorting even. In some embodiments, the gating procedure is not fixed but is determined based one the population obtained during each sorting event is a CD3+ gating procedure.

In some embodiments, the gating and compensation for the mean fluorescence intensity (MFI) is in the range of about 0.5%±0.25%. In some embodiments, the gating and compensation for the mean fluorescence intensity (MFI) is in the range of about 1.75%±0.25%. In some embodiments, the gating and compensation for the mean fluorescence intensity (MFI) is in the range of about 1.75%±0.25% when setting the TIGIT high gate with PBMC's. In some embodiments, the MFI calculation employs the mean value measured from 1, 2, 3, or 4, or more lots or batches of PBMCs. In some embodiments, the MFI calculation employs the median value measured from 1, 2, 3, or 4, or more lots or batches of PBMCs.

In some embodiments, the gating and compensation methods of FACS sorting which are used to determine the mean fluorescence intensity (MFI) are adjusted such that the MFI of TIGIT for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the TIGIT high gate. In some embodiments, the MFI calculation is the mean value measured using at least two samples of PBMCs. In some embodiments, the MFI calculation is the median value measured using at least two samples of PBMCs.

In some embodiments, the TIGIT positive (TIGIT+) cells are sorted by FACs and/or other flow cytometry method. In some embodiments, the TIGIT positive TILs are TIGIThigh TILs. In some embodiments, the TIGIT positive TILs are TIGITintermediate TILs. In some embodiments, the TIGIT+ cells are sorted by employing a bead selection method. In some embodiments, the TIGIT+ cells are sorted by employing a magnetic bead selection method. In some embodiments, the bead selection employs an antibody bound bead, for example but no limited to a commercially available bead, such as Miltenyi or Fisher, for selection. In some embodiments, the TIGIT+ high cells are sorted by employing a bead selection method. In some embodiments, the TIGIT+ high cells are sorted by employing a magnetic bead selection method. In some embodiments, the bead selection employs an antibody bound bead, for example but no limited to a commercially available bead, such as Miltenyi or Fisher, for selection. In some embodiments, the anti-TIGIT antibody is conjugated to the bead, either directly or indirectly. In some embodiments, the bead selection process selects for both TIGIT+ and CD3+ TILs. In some embodiments the anti-TIGIT antibody includes, e.g., but is not limited to tiragolumab (anti-TIGIT, RG6058), BMS-986207, OMP-313M32, BGB-A1217, IBI939, COM902, EOS884448 (EOS-448), etigilimab, MK-7684, and/or AB154.

In some embodiments, the collection buffer employed to collect the TIGIT+ cells and/or the TIGIT negative cells does not include serum. In some embodiments, the collection buffer employed to collect the TIGIT+ cells and/or the TIGIT negative cells includes serum. In some embodiments, the collection buffer employed to collect the TIGIT+ cells and/or the TIGIT negative cells includes a component to mitigate or reduce viscosity differences between sort buffer and downstream buffers and/or media. In some embodiments, the collection buffer employed to collect the TIGIT+ cells and/or the TIGIT negative cells includes only human serum albumin (HSA). In some embodiments, the collection buffer employed to collect the TIGIT+ cells and/or the TIGIT negative cells includes an equal amount of HSA and PBS/EDTA Buffer. In some embodiments, the collection buffer employed to collect the TIGIT+ cells and/or the TIGIT negative cells includes HSA and PBS/EDTA Buffer at a 1:1, 1:2, 1:3, 1:4, 2:1, 3:1, or 4:1 ratio.

In some embodiments, preselection involves selecting TIGIT positive TILs from the first population of TILs to obtain a TIGIT enriched TIL population comprises the selecting a population of TILs from a first population of TILs that are at least 11.27% to 74.4% TIGIT positive TILs. In some embodiments, the first population of TILs are at least 20% to 80% TIGIT positive TILs, at least 20% to 80% TIGIT positive TILs, at least 30% to 80% TIGIT positive TILs, at least 40% to 80% TIGIT positive TILs, at least 50% to 80% TIGIT positive TILs, at least 10% to 70% TIGIT positive TILs, at least 20% to 70% TIGIT positive TILs, at least 30% to 70% TIGIT positive TILs, or at least 40% to 70% TIGIT positive TILs.

In some embodiments, the selection step (e.g., preselection and/or selecting TIGIT positive cells) comprises the steps of:
(i) exposing the first population of TILs and a population of PBMC to an excess of a monoclonal anti-TIGIT IgG4 antibody that binds to TIGIT through an N-terminal loop outside the IgV domain of TIGIT,
(ii) adding an excess of an anti-IgG4 antibody conjugated to a fluorophore,
(iii) obtaining the TIGIT enriched TIL population based on the intensity of the fluorophore of the TIGIT positive TILs in the first population of TILs compared to the intensity in the population of PBMCs as performed by fluorescence-activated cell sorting (FACS).

In some embodiments, the TIGIT positive TILs are TIGIThigh TILs.

In some embodiments, at least 70% of the TIGIT enriched TIL population are TIGIT positive TILs. In some embodiments, at least 80% of the TIGIT enriched TIL population are TIGIT positive TILs. In some embodiments, at least 90% of the TIGIT enriched TIL population are TIGIT positive TILs. In some embodiments, at least 95% of the TIGIT enriched TIL population are TIGIT positive TILs. In some embodiments, at least 99% of the TIGIT enriched TIL population are TIGIT positive TILs. In some embodiments, 100% of the TIGIT enriched TIL population are TIGIT positive TILs.

In some embodiments, the selection of TIGIT positive TILs occurs until there are at least 1×10⁴ TILs TIGIT positive TILs, at least 1×10⁵ TILs TIGIT positive TILs, at least 1×10⁶ TILs TIGIT positive TILs, at least 1×10⁷ TILs TIGIT positive TILs, at least 1×10⁸ TILs TIGIT positive TILs. In some embodiments; the selection of TIGIT positive TILs occurs until there are at least 1×10⁶ TILs TIGIT positive TILs.

In some embodiments, the selection step, exemplified as Step A2 of FIG. 1, comprises the steps of (i) exposing the first population of TILs to an excess of a monoclonal anti-TIGIT IgG4 antibody that binds to TIGIT through an N-terminal loop outside the IgV domain of TIGIT, (ii) adding an excess of an anti-IgG4 antibody conjugated to a fluorophore, and (iii) performing a flow-based cell sort based on the fluorophore to obtain a TIGIT enriched TIL population. In some embodiments, the monoclonal anti-TIGIT IgG4 antibody is nivolumab or variants, fragments, or conjugates thereof. In some embodiments, the anti-IgG4 antibody is clone anti-human IgG4, Clone HP6023. In some embodiments, the anti-TIGIT antibody for use in the selection in step (b) binds to the same epitope as EH12.2H7 or nivolumab.

To determine if TILs derived from a tumor sample are TIGIThigh, one skilled in the art can utilize a reference value corresponding to the level of expression of TIGIT in peripheral T cells obtained from a blood sample from one or more healthy human subjects. TIGIT positive cells in the reference sample can be defined using fluorescence minus one controls and matching isotype controls. In some embodiments, the expression level of TIGIT is measured in CD3+/TIGIT+ peripheral T cells from a healthy subject (e.g., the reference cells) is used to establish a threshold value or cut-off value of immunostaining intensity of TIGIT in TILs obtained from a tumor. The threshold value can be defined as the minimal intensity of TIGIT immunostaining of TIGIThigh T cells. As such, TILs with a TIGIT expression that is the same or above the threshold value can be considered to be TIGIThigh cells. In some instances, the TIGIThigh TILs represent those with the highest intensity of TIGIT immunostaining corresponding to a maximum 1% or less of the total CD3+ cells. In other instances, the TIGIThigh TILs represent those with the highest intensity of TIGIT immunostaining corresponding to the maximum 0.75% or less of the total CD3+ cells. In some instances, the TIGIThigh TILs represent those with the highest intensity of TIGIT immunostaining corresponding to the maximum 0.50% or less of the total CD3+ cells. In one instance, the TIGIThigh TILs represent those with the highest intensity of TIGIT immunostaining corresponding to the maximum 0.25% or less of the total CD3+ cells.

In some embodiments, the PD-1 gating method of WO2019156568 is employed for TIGIT. To determine if TILs derived from a tumor sample are TIGIThigh, one skilled in the art can utilize a reference value corresponding to the level of expression of TIGIT in peripheral T cells obtained from a blood sample from one or more healthy human subjects. TIGIT positive cells in the reference sample can be defined using fluorescence minus one controls and matching isotype controls. In some embodiments, the expression level of TIGIT is measured in CD3+/TIGIT+ peripheral T cells from a healthy subject (e.g., the reference cells) is used to establish a threshold value or cut-off value of immunostaining intensity of TIGIT in TILs obtained from a tumor. The threshold value can be defined as the minimal intensity of TIGIT immunostaining of TIGIThigh T cells. As such, TILs with a TIGIT expression that is the same or above the threshold value can be considered to be TIGIThigh cells. In some instances, the TIGIThigh TILs represent those with the highest intensity of TIGIT immunostaining corresponding to a maximum 1% or less of the total CD3+ cells. In other instances, the TIGIThigh TILs represent those with the highest intensity of TIGIT immunostaining corresponding to the maximum 0.75% or less of the total CD3+ cells. In some instances, the TIGIThigh TILs represent those with the highest intensity of TIGIT immunostaining corresponding to the maximum 0.50% or less of the total CD3+ cells. In one instance, the TIGIThigh TILs represent those with the highest intensity of TIGIT immunostaining corresponding to the maximum 0.25% or less of the total CD3+ cells.

In some embodiments, the TIGIT positive (TIGIT+) cells selected can be frozen prior to proceeding with the priming first expansion, for example, Step B of FIG. 1 (in particular, e.g.,

FIG. 1B and/or FIG. 1C and/or FIG. 1D and/or FIG. 1E and/or FIG. 1F and/or FIG. 1G and/or FIG. 1H).

a. Fluorophores

In some embodiments, the primary cell population TILs are stained with a cocktail that includes an anti-TIGIT antibody linked to a fluorophore and an anti-CD3 antibody linked to a fluorophore. In some embodiments, the primary cell population TILs are stained with a cocktail that includes an anti-TIGIT antibody linked to a fluorophore (for example, PE, live/dead violet) and anti-CD3-FITC. In some embodiments, the primary cell population TILs are stained with a cocktail that includes anti-TIGIT-PE, anti-CD3-FITC and live/dead blue stain (ThermoFisher, MA, Cat #L23105). In some embodiments, the after incubation with the anti-PD1 antibody, TIGIT positive cells are selected for expansion according to the priming first expansion a described herein, for example, in Step B.

In some embodiments, the fluorophore includes, but is not limited to PE (Phycoerythrin), APC (allophycocyanin), PerCP (peridinin chlorophyll protein), DyLight 405, Alexa Fluor 405, Pacific Blue, Alexa Fluor 488, FITC (fluorescein isothiocyanate), DyLight 550, Alexa Fluor 647, DyLight 650, and Alexa Fluor 700. In some embodiments, the flurophore includes, but is not limited to PE-Alexa Fluor® 647, PE-Cy5, PerCP-Cy5.5, PE-Cy5.5, PE-Alexa Fluor® 750, PE-Cy7, and APC-Cy7. In some embodiments, the flurophore includes, but is not limited to a fluorescein dye. Examples of fluorescein dyes include, but are not limited to, 5-carboxyfluorescein, fluorescein-5-isothiocyanate and 6-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carbxoyfluorescein quaternary ammonium, carboxyfluorescein phosphonate, carboxyfluorescein GABA, 5′(6′)-carboxyfluorescein, carboxyfluorescein-cys-Cy5, and fluorescein glutathione. In some embodiments, the fluorescent moiety is a rhodamine dye. Examples of rhodamine dyes include, but are not limited to, tetramethylrhodamine-6-isothiocyanate, 5-carboxytetramethylrhodamine, 5-carboxy rhodol derivatives, carboxy rhodamine 110, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). In some embodiments, the fluorescent moiety is a cyanine dye. Examples of cyanine dyes include, but are not limited to, Cy3, Cy3B, Cy3.5, Cy5, Cy5.5, and Cy 7.

13. Multiple Preselection Selection (as exemplified in Step A2 of FIG. 1)

In some embodiments, the TILs are preselected for being PD-1 positive (PD-1+) prior to the priming first expansion. In some embodiments, the TILs are preselected for being CD39 positive (CD39+) prior to the priming first expansion. In some embodiments, the TILs are preselected for being CD38 positive (CD38+) prior to the priming first expansion. In some embodiments, the TILs are preselected for being CD103 positive (CD103+) prior to the priming first expansion. In some embodiments, the TILs are preselected for being LAG3 positive (LAG3+) prior to the priming first expansion. In some embodiments, the TILs are preselected for being TIM3 positive (TIM3+) prior to the priming first expansion. In some embodiments, the TILs are preselected for being TIGIT positive (TIGIT+) prior to the priming first expansion.

In some embodiments, the TILs are preselected for being PD-1 positive (PD-1+) and CD39 positive (CD39+) prior to the priming first expansion. In some embodiments; the TILs are preselected for being PD-1 positive (PD-1+) and CD103 positive (CD103+) prior to the priming first expansion. In some embodiments, the TILs are preselected for being CD39 positive (CD39+) and CD103 positive (CD103+) prior to the priming first expansion. In some embodiments, the TILs are preselected for being CD39 positive (CD39+) and CD101 positive (CD101+) prior to the priming first expansion. In some embodiments, the TILs are preselected for being PD-1 positive (PD-1+), CD39 positive (CD39+), and CD103 positive (CD103+) prior to the priming first expansion. In some embodiments, the TILs are preselected for being PD-1 positive (PD-1+) and CD101 positive (CD101+) prior to the priming first expansion. In some embodiments, the TILs are preselected for being CD39 positive (CD39+) and CD103 positive (CD103+) prior to the priming first expansion. In some embodiments, the TILs are preselected for being PD-1 positive (PD-1+), CD39 positive (CD39+), CD103 positive (CD103+), and CD101 positive (CD101+) prior to the priming first expansion. In some embodiments, the TILs are preselected for being CD39 positive (CD39+), CD103 positive (CD103+), and CD101 positive (CD101+) prior to the priming first expansion. In some embodiments, the TILs are preselected for being PD-1 positive (PD-1+) and LAG3 positive (LAG3+ positive) prior to the priming first expansion. In some embodiments, the TILs are preselected for being PD-1 positive (PD-1+) and TIM3 positive (TIM3+) prior to the priming first expansion. In some embodiments, the TILs are preselected for being PD-1 positive (PD-1+) and TIGIT positive (TIGIT+) prior to the priming first expansion.

In some embodiments, the TILs are preselected for being PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and/or TIGIT positive (TIGIT+) prior to the priming first expansion. In some embodiments, the TILs are preselected for being positive for any two of PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and/or TIGIT positive (TIGIT+) prior to the priming first expansion. In some embodiments, the TILs are preselected for being positive for any three of PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and/or TIGIT positive (TIGIT+) prior to the priming first expansion. In some embodiments, the TILs are preselected for being positive for any four of PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and/or TIGIT positive (TIGIT+) prior to the priming first expansion. In some embodiments, the TILs are preselected for being positive for any five of PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and/or TIGIT positive (TIGIT+) prior to the priming first expansion. In some embodiments, the TILs are preselected for being positive for any six of PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and/or TIGIT positive (TIGIT+) prior to the priming first expansion. In some embodiments, the TILs are preselected for being positive for all of PD-1 positive (PD-1+), CD39 positive (CD39+), CD38 positive (CD38+), CD103 positive (CD103+), CD101 positive (CD101+), LAG3 positive (LAG3+ positive), TIM3 positive (TIM3+), and TIGIT positive (TIGIT+) prior to the priming first expansion.

In some embodiments, the TILs are preselected for being PD-1 positive (PD-1+), LAG3 positive (LAG3+ positive), CD38 positive (CD38+), and CD101 positive (CD101+). In some embodiments, the TILs are preselected for being PD-1high, LAG3high, CD38lo, and CD101lo. In some embodiments, the TILs are preselected for being PD-1 positive (PD-1+), LAG3 positive (LAG3+ positive), and CD38 positive (CD38+). In some embodiments, the TILs are preselected for being PD-1high, LAG3high, and CD38lo. In some embodiments, the TILs are preselected for being PD-1 positive (PD-1+), LAG3 positive (LAG3+ positive), and CD101 positive (CD101+). In some embodiments, the TILs are preselected for being PD-1high, LAG-3high, and CD101lo. In some embodiments, the TILs are preselected for being PD-1 positive (PD-1+) and CD38 positive (CD38+). In some embodiments, the TILs are preselected for being PD-1high and CD38lo. In some embodiments, the TILs are preselected for being PD-1 positive (PD-1+) and CD101 positive (CD101+). In some embodiments, the TILs are preselected for being PD-1high and CD101lo.

a. Fluorescence Methods/Assays

The present invention provides methods, including for example flow cytometry methods such as FACS, wherein the assays employ fluorescently labeled antibodies for detection. The fluorochromes which can be used in these assays and embodiment are well known in the art. In some embodiments, fluorochromes include, for example, but are not limited to PE, APC, PE-Cy5, Alexa Fluor 647, PE-Cy-7, PerCP-Cy5.5, Alexa Fluor 488, Pacific Blue, FITC, AmCyan, APC-Cy7, PerCP, and APC-H7.

In some embodiments, the PD-1 gating method of WO2019156568 is employed for PD-1, CD39, CD38, CD103, CD101, LAG3, TIM3 and/or TIGIT. To determine if TILs derived from a tumor sample are “high”, one skilled in the art can utilize a reference value corresponding to the level of expression of PD-1, CD39, CD38, CD103, CD101, LAG3, TIM3 and/or TIGIT in peripheral T cells obtained from a blood sample from one or more healthy human subjects. PD-1, CD39, CD38, CD103, CD101, LAG3, TIM3 and/or TIGIT positive cells in the reference sample can be defined using fluorescence minus one controls and matching isotype controls. In some embodiments, the expression level of PD-1, CD39, CD38, CD103, CD101, LAG3, TIM3 and/or TIGIT is measured in CD3+/(PD-1+, CD39+, CD38+, CD103+, CD101+, LAG3+, TIM3+ and/or TIGIT+) peripheral T cells from a healthy subject (e.g., the reference cells) is used to establish a threshold value or cut-off value of immunostaining intensity of PD-1, CD39, CD38, CD103, CD101, LAG3, TIM3 and/or TIGIT in TILs obtained from a tumor. The threshold value can be defined as the minimal intensity of PD-1, CD39, CD38, CD103, CD101, LAG3, TIM3 and/or TIGIT immunostaining of PD-1high, CD39 high, CD38high, CD103high, CD101high, LAG3high, TIM3high and/or TIGIThigh T cells. As such, TILs with a PD-1, CD39, CD38, CD103, CD101, LAG3, TIM3 and/or TIGIT expression that is the same or above the threshold value can be considered to be PD-1high, CD39 high, CD38high, CD103high, CD101high, LAG3high, TIM3high and/or TIGIThigh cells. In some instances, the PD-1high, CD39 high, CD38high, CD103high, CD101high, LAG3high, TIM3high and/or TIGIThigh TILs represent those with the highest intensity of PD-1, CD39, CD38, CD103, CD101, LAG3, TIM3 and/or TIGIT immunostaining corresponding to a maximum 1% or less of the total CD3+ cells. In other instances, the PD-1high, CD39 high, CD38high, CD103high, CD101high, LAG3high, TIM3high and/or TIGIThigh TILs represent those with the highest intensity of PD-1, CD39, CD38, CD103, CD101, LAG3, TIM3 and/or TIGIT immunostaining corresponding to the maximum 0.75% or less of the total CD3+ cells. In some instances, the PD-1high, CD39 high, CD38high, CD103high, CD101high, LAG3high, TIM3high and/or TIGIThigh TILs represent those with the highest intensity of PD-1, CD39, CD38, CD103, CD101, LAG3, TIM3 and/or TIGIT immunostaining corresponding to the maximum 0.50% or less of the total CD3+ cells. In one instance, the PD-1high, CD39 high, CD38high, CD103high, CD101high, LAG3high, TIM3high and/or TIGIThigh TILs represent those with the highest intensity of PD-1, CD39, CD38, CD103, CD101, LAG3, TIM3 and/or TIGIT immunostaining corresponding to the maximum 0.25% or less of the total CD3+ cells.

In some embodiments, quantifying the number of PD-1+, CD39+, CD38+, CD103+, CD101+, LAG3+, TIM3+ and/or TIGIT+ TILs, wherein the positive TILs exhibit an intensity of immunostaining for PD-1, CD39, CD38, CD103, CD101, LAG3, TIM3 and/or TIGIT, respectively, that is equal or higher than a pre-determined reference value 1 (for example, referred to as REF1), relative to the total number of TILs in the tumor sample or to the total number of PBMCs in a reference sample, to obtain the percent (%) of PD-1+, CD39+, CD38+, CD103+, CD101+, LAG3+, TIM3+ and/or TIGIT+ TILs in the tumor sample,

In some embodiments, the method for establishing a pre-determined reference value 1 (REF1) of intensity of immunostaining for PD-1 comprises the steps of i) providing at least one blood sample from a healthy human donor, ii) isolating peripheral blood mononuclear cells (PBMC) from said blood sample; iii) incubating the PBMC sample of step (ii) under conditions allowing specific antigen-antibody binding with an (e.g., labeled) antibody directed to human T lymphocyte marker (M, such as CD3) and with a labeled antibody directed to PD-1, CD39, CD38, CD103, CD101, LAG3, TIM3 and/or TIGIT to allow detection of M+ cells, including PD-1+, CD39+, CD38+, CD103+, CD101+, LAG3+, TIM3+ and/or TIGIT⁺ T cells and M+/(PD-1+, CD39+, CD38+, CD103+, CD101+, LAG3+, TIM3+ and/or TIGIT) T cells in said PBMC sample; iv) measuring the intensity of immunostaining of PD-1 for M+/(PD-1+, CD39+, CD38+, CD103+, CD101+, LAG3+, TIM3+ and/or TIGIT⁺ T cells of step (iii); v) selecting the upper 0.50% to upper 1.5%, preferably the upper 1% of the M+/(PD-1+, CD39+, CD38+, CD103+, CD101+, LAG3+, TIM3+ and/or TIGIT+) T cells of step (iv) having the highest intensity of immunostaining for PD-1; and vi) determining the minimal intensity of immunostaining for PD-1 in the upper 0.50% to upper 1.5%, preferably in the upper 1.0% of the M+/(PD-1+, CD39+, CD38+, CD103+, CD101+, LAG3+, TIM3+ and/or TIGIT+) T cells of step (v) thereby establishing the pre-determined reference (REF) or base-line value.

In this embodiment, the use of fluorochromic agents attached to anti-PD-1, anti-CD39, anti-CD38, anti-CD103, anti-CD101, anti-LAG3, anti-TIM3 and/or anti-TIGIT antibodies to enable the flow cytometer to sort on the basis of size, granularity and fluorescent light is highly advantageous. Thus, the flow cytometer can be configured to provide information about the relative size (forward scatter or “FSC”), granularity or internal complexity (side scatter or “SSC”), and relative fluorescent intensity of the cell sample. The fluorescent light sorts on the basis of PD-1-expressing, CD39—expressing, CD38—expressing, CD103—expressing, CD101—expressing, LAG3-expressing, TIM3—expressing, and/or TIGIT-expressing, enabling the cytometer to identify and enrich for these monocytes.

In some embodiments, the fluorescence method or assay is employed as part of a method for expanding tumor infiltrating lymphocytes (TILs) into a therapeutic population of TILs. The method comprises the following steps:
(a) obtaining and/or receiving a first population of TILs from a tumor sample resected from a subject and digested to produce a tumor digest comprising the first population of TILs;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population, wherein at least a range of 0.5% to 90% of the first population of TILs are PD-1, CD39, CD38, CD103, CD101, LAG3, TIM3 and/or TIGIT positive TILs;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs;
(d) performing a rapid second expansion by supplementing the cell culture medium of the second population of TILs with additional IL-2, OKT-3, and APCs, to produce a third population of TILs, wherein the number of APCs added in the rapid second expansion is at least twice the number of APCs added in step (b), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(e) harvesting therapeutic population of TILs obtained from step (d); and
(f) transferring the harvested TIL population from step (e) to an infusion bag.

In some embodiments, the selecting in step (b) comprises selecting at least a range of 0.5% to 90% of the first population of TILs are PD-1, CD39, CD38, CD103, CD101, LAG3, TIM3 and/or TIGIT positive TILs. In some embodiments, the selecting in step (b) comprises selecting at least a range of 0.5% to 80% of the first population of TILs are PD-1, CD39, CD38, CD103, CD101, LAG3, TIM3 and/or TIGIT positive TILs. In some embodiments, the selecting in step (b) comprises selecting at least a range of 0.5% to 70% of the first population of TILs are PD-1, CD39, CD38, CD103, CD101, LAG3, TIM3 and/or TIGIT positive TILs. In some embodiments, the selecting in step (b) comprises selecting at least a range of 0.5% to 60% of the first population of TILs are PD-1, CD39, CD38, CD103, CD101, LAG3, TIM3 and/or TIGIT positive TILs. In some embodiments, the selecting in step (b) comprises selecting at least a range of 0.5% to 50% of the first population of TILs are PD-1, CD39, CD38, CD103, CD101, LAG3, TIM3 and/or TIGIT positive TILs. In some embodiments, the selecting in step (b) comprises selecting at least a range of 1% to 90% of the first population of TILs are PD-1, CD39, CD38, CD103, CD101, LAG3, TIM3 and/or TIGIT positive TILs. In some embodiments, the selecting in step (b) comprises selecting at least a range of 1% to 80% of the first population of TILs are PD-1, CD39, CD38, CD103, CD101, LAG3, TIM3 and/or TIGIT positive TILs. In some embodiments, the selecting in step (b) comprises selecting at least a range of 5% to 90% of the first population of TILs are PD-1, CD39, CD38, CD103, CD101, LAG3, TIM3 and/or TIGIT positive TILs. In some embodiments, the selecting in step (b) comprises selecting at least a range of 5% to 80% of the first population of TILs are PD-1, CD39, CD38, CD103, CD101, LAG3, TIM3 and/or TIGIT positive TILs. In some embodiments, the selecting in step (b) comprises selecting at least a range of 10% to 90% of the first population of TILs are PD-1, CD39, CD38, CD103, CD101, LAG3, TIM3 and/or TIGIT positive TILs. In some embodiments, the selecting in step (b) comprises selecting at least a range of 10% to 80% of the first population of TILs are PD-1, CD39, CD38, CD103, CD101, LAG3, TIM3 and/or TIGIT positive TILs. In some embodiments, the selecting in step (b) comprises selecting at least a range of 15% to 90% of the first population of TILs are PD-1, CD39, CD38, CD103, CD101, LAG3, TIM3 and/or TIGIT positive TILs. In some embodiments, the selecting in step (b) comprises selecting at least a range of 15% to 80% of the first population of TILs are PD-1, CD39, CD38, CD103, CD101, LAG3, TIM3 and/or TIGIT positive TILs. In some embodiments, the selecting in step (b) comprises selecting at least a range of 20% to 90% of the first population of TILs are PD-1, CD39, CD38, CD103, CD101, LAG3, TIM3 and/or TIGIT positive TILs. In some embodiments, the selecting in step (b) comprises selecting at least a range of 20% to 80% of the first population of TILs are PD-1, CD39, CD38, CD103, CD101, LAG3, TIM3 and/or TIGIT positive TILs. In some embodiments, the selecting in step (b) comprises selecting at least a range of 30% to 90% of the first population of TILs are PD-1, CD39, CD38, CD103, CD101, LAG3, TIM3 and/or TIGIT positive TILs. In some embodiments, the selecting in step (b) comprises selecting at least a range of 30% to 80% of the first population of TILs are PD-1, CD39, CD38, CD103, CD101, LAG3, TIM3 and/or TIGIT positive TILs. In some embodiments, the selecting in step (b) comprises selecting at least a range of 40% to 90% of the first population of TILs are PD-1, CD39, CD38, CD103, CD101, LAG3, TIM3 and/or TIGIT positive TILs. In some embodiments, the selecting in step (b) comprises selecting at least a range of 40% to 80% of the first population of TILs are PD-1, CD39, CD38, CD103, CD101, LAG3, TIM3 and/or TIGIT positive TILs.

In some embodiments, the selection of step (b) comprises the steps of:
(i) exposing the first population of TILs and a population of PBMC to an excess of an anti-PD-1, anti-CD39, anti-CD38, anti-CD103, anti-CD101, anti-LAG3, anti-TIM3 and/or anti-TIGIT antibody that binds to PD-1, CD39, CD38, CD103, CD101, LAG3, TIM3 and/or TIGIT,
(ii) adding an excess of an anti-IgG4 antibody (or other antibody that binds to the anti-PD-1, anti-CD39, anti-CD38, anti-CD103, anti-CD101, anti-LAG3, anti-TIM3 and/or anti-TIGIT) conjugated to a fluorophore,
(iii) obtaining the PD-1, CD39, CD38, CD103, CD101, LAG3, TIM3 and/or TIGIT enriched TIL population based on the intensity of the fluorophore of the PD-1, CD39, CD38, CD103, CD101, LAG3, TIM3 and/or TIGIT positive TILs in the first population of TILs compared to the intensity in the population of PBMCs as performed by fluorescence-activated cell sorting (FACS).

In some embodiments, the intensity of the fluorophore in both the first population and the population of PBMCs is used to set up FACS gates for establishing low, medium, and high levels of intensity that correspond to negative, intermediate and high TILs positive for any one or more of PD-1, CD39, CD103, CD101, LAG3, TIM3 and/or TIGIT.

In some embodiments, the FACS gates are set-up after step (a).

In some embodiments, the PD-1 positive TILs are PD-1high TILs. In some embodiments, the PD-1, LAG3, TIM3 and/or TIGIT positive TILs are PD-1high, LAG3high, TIM3high and/or TIGIThigh TILs. In some embodiments, at least 80% of the PD-1 enriched TIL population are PD-1 positive TILs. In some embodiments, at least 80% of the PD-1, LAG3, TIM3 and/or TIGIT enriched TIL population are PD-1, LAG3, TIM3 and/or TIGIT positive TILs.

In some embodiments, the intensity of the fluorophore in both the first population and the population of PBMCs is used to set up FACS gates for establishing low, medium, and high levels of intensity that correspond to PD-1, CD39, CD38, CD103, CD101, LAG3, TIM3 and/or TIGIT negative TILs, PD-1, CD39, CD38, CD103, CD101, LAG3, TIM3 and/or TIGIT intermediate TILs, and PD-1, CD39, CD38, CD103, CD101, LAG3, TIM3 and/or TIGIT positive TILs, respectively. In some embodiments, the FACS gates are set-up after step (a). In some embodiments, the PD-1, CD39, CD38, CD103, CD101, LAG3, TIM3 and/or TIGIT positive TILs are PD-1high, LAG3high, TIM3high and/or TIGIThigh TILs. In some embodiments, at least 80% of the PD-1, CD39, LAG3, TIM3 and/or TIGIT enriched TIL population are PD-1, CD39, CD38, CD103, CD101, LAG3, TIM3 and/or TIGIT positive TILs. In some embodiments, at least 80% of the PD-1 enriched TIL population are PD-1 positive TILs, at least 80% of the LAG3 enriched TIL population are LAG3 positive TILs, at least 80% of the TIM3 enriched TIL population are TIM3 positive TILs, and/or at least 80% of the TIGIThigh enriched TIL population are PD-1 positive TILs.

In some embodiments, the intensity of the fluorophore can be measured by direct intensity or normalized intensity. In some embodiments, the intensity is compared to a control or reference intensity. In some embodiments, the fluorescence intensity is increased by at least 5%, 10%, 15%, 20%, 25%, 30%, 35%, 40%, 45%, 50%, 55%, 60%, 65%, 70%, 75%, 80%, 85%, 90%, 95%, 100%. 150%, 200%, 250%. 300%, 350%, 400%, 450%, 500% or more as compared to a reference or control intensity. In some embodiments, a fluorescence intensity increase of least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% or more as compared to a reference or control intensity is indicative of a positive TIL population for the reference marker.

In some embodiments, the intensity of the fluorophore can be measured by direct intensity or normalized intensity. In some embodiments, the intensity is compared to a control or reference intensity. In some embodiments, the fluorescence intensity is increased by at least one-fold, two-fold, three-fold, four-fold, five-fold, six-fold, seven-fold, eight-fold, nine-fold, ten-fold, fifteen-fold, twenty-fold or more as compared to a reference or control intensity. In some embodiments, a fluorescence intensity increase of least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% or more as compared to a reference or control intensity is indicative of a positive TIL population for the reference marker.

In some embodiments, the gating and compensation methods of FACS sorting which are used to determine the mean fluorescence intensity (MFI) are adjusted such that the MFI of PD-1, CD39, CD38, CD103, CD101, LAG3, TIM3 and/or TIGIT for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the PD-1, CD39, CD38, CD103, CD101, LAG3, TIM3 and/or TIGIT high gate. In some embodiments, the MFI calculation is the mean value measured using at least two samples of PBMCs. In some embodiments, the MFI calculation is the median value measured using at least two samples of PBMCs.

In some embodiments, the gating and compensation methods of FACS sorting which are used to determine the mean fluorescence intensity (MFI) are adjusted such that the MFI of PD-1 for the control PBMCs (e.g., PBMCs from a healthy donor) is in the range of about 0.5% to 2.0% (e.g., about 0.5%, 0.55%, 0.6%, 0.65%, 0.7%, 0.75%, 0.8%, 0.85%, 0.9%, 0.95%, 1.0%, 1.05% 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%) for the PD-1 high gate. In some embodiments, the MFI calculation is the mean value measured using at least two samples of PBMCs. In some embodiments, the MFI calculation is the median value measured using at least two samples of PBMCs.

In some embodiments, the PD-1high expression, LAG3 high expression, TIM3 high expression and/or TIGIT high expression is determined by flow cytometry using minimum cutoff for normalized fluorescence intensity selected from the group consisting of about 80%, about 70%, about 60%, about 50%, about 40%, about 30%, about 20%, or about 10%.

The control, control value, reference, reference value, or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. The reference value can be based on a large number of samples, such as from population of subjects of the chronological age matched group, or based on a pool of samples including or excluding the sample to be tested.

B. STEP B: Priming First Expansion

In some embodiments, the present methods provide for younger TILs, which may provide additional therapeutic benefits over older TILs (i.e., TILs which have further undergone more rounds of replication prior to administration to a subject/patient). Features of young TILs have been described in the literature, for example Donia, at al., Scandinavian Journal of Immunology, 75:157-167 (2012); Dudley et al., Clin Cancer Res, 16:6122-6131 (2010); Huang et al., J Immunother, 28(3):258-267 (2005); Besser et al., Clin Cancer Res, 19 (17):OF1-OF9 (2013); Besser et al., J Immunother 32:415-423 (2009); Robbins, et al., J Immunol 2004; 173:7125-7130; Shen et al., J Immunother, 30:123-129 (2007); Zhou, et al., J Immunother, 28:53-62 (2005); and Tran, et al., J Immunother, 31:742-751 (2008), all of which are incorporated herein by reference in their entireties. In some embodiments, the process is a process as provided in FIG. 1A, as well as described in PCT/US2018/012633.

After dissection or digestion (for example to obtain whole tumor digests and/or whole tumor cell suspensions) of tumor fragments and/or tumor fragments, for example such as described in Step A of FIG. 1 (in particular, e.g., FIG. 1B and/or FIG. 1C and/or FIG. 1D and/or FIG. 1E and/or FIG. 1F and/or FIG. 1G and/or FIG. 1H), the resulting cells are cultured in serum containing IL-2, OKT-3, and feeder cells (e.g., antigen-presenting feeder cells or allogenic irradiated PBMCs), under conditions that favor the growth of TILs over tumor and other cells. In some embodiments, the IL-2, OKT-3, and feeder cells are added at culture initiation along with the tumor digest and/or tumor fragments (e.g., at Day 0). In some embodiments, the tumor digests and/or tumor fragments are incubated in a container with up to 60 fragments (in embodiments where fragments are employed) per container and with 6000 IU/mL of IL-2. In some embodiments, this primary cell population is cultured for a period of days, generally from 1 to 8 days, resulting in a bulk TIL population, generally about 1×10⁸ bulk TIL cells. In some embodiments, this period is referred to activation I. In some embodiments, this primary cell population is cultured for a period of days, generally from 1 to 7 days, resulting in a bulk TIL population, generally about 1×10⁸ bulk TIL cells. In some embodiments, priming first expansion occurs for a period of 1 to 8 days, resulting in a bulk TIL population, generally about 1×10⁸ bulk TIL cells. In some embodiments, priming first expansion occurs for a period of 1 to 7 days, resulting in a bulk TIL population, generally about 1×10⁸ bulk TIL cells. In some embodiments, priming first expansion occurs for a period of 1 to 3 days, resulting in a bulk TIL population, generally about 1×10⁸ bulk TIL cells. In some embodiments, priming first expansion occurs for a period of 1 to 4 days, resulting in a bulk TIL population, generally about 1×10⁸ bulk TIL cells. In some embodiments, priming first expansion occurs for a period of 1 to 5 days, resulting in a bulk TIL population, generally about 1×10⁸ bulk TIL cells. In some embodiments, priming first expansion occurs for a period of 1 to 6 days, resulting in a bulk TIL population, generally about 1×10⁸ bulk TIL cells. In some embodiments, this priming first expansion occurs for a period of 5 to 8 days, resulting in a bulk TIL population, generally about 1×10⁸ bulk TIL cells. In some embodiments, this priming first expansion occurs for a period of 5 to 7 days, resulting in a bulk TIL population, generally about 1×10⁸ bulk TIL cells. In some embodiments, this priming first expansion occurs for a period of about 6 to 8 days, resulting in a bulk TIL population, generally about 1×10⁸ bulk TIL cells. In some embodiments, this priming first expansion occurs for a period of about 6 to 7 days, resulting in a bulk TIL population, generally about 1×10⁸ bulk TIL cells. In some embodiments, this priming first expansion occurs for a period of about 7 to 8 days, resulting in a bulk TIL population, generally about 1×10⁸ bulk TIL cells. In some embodiments, this priming first expansion occurs for a period of about 7 days, resulting in a bulk TIL population, generally about 1×10⁸ bulk TIL cells. In some embodiments, this priming first expansion occurs for a period of about 8 days, resulting in a bulk TIL population, generally about 1×10⁸ bulk TIL cells.

In some embodiments, this priming first expansion occurs for a period of about 6 to 11 days, resulting in a bulk TIL population, generally about 1×10⁸ bulk TIL cells. In some embodiments, this priming first expansion occurs for a period of about 7 to 11 days, resulting in a bulk TIL population, generally about 1×10⁸ bulk TIL cells. In some embodiments, this priming first expansion occurs for a period of about 8 to 11 days, resulting in a bulk TIL population, generally about 1×10⁸ bulk TIL cells. In some embodiments, this priming first expansion occurs for a period of about 9 to 11 days, resulting in a bulk TIL population, generally about 1×10⁸ bulk TIL cells. In some embodiments, this priming first expansion occurs for a period of about 10 to 11 days, resulting in a bulk TIL population, generally about 1×10⁸ bulk TIL cells. In some embodiments, this priming first expansion occurs for a period of about 9 days, resulting in a bulk TIL population, generally about 1×10⁸ bulk TIL cells. In some embodiments, this priming first expansion occurs for a period of about 10 days, resulting in a bulk TIL population, generally about 1×10⁸ bulk TIL cells. In some embodiments, this priming first expansion occurs for a period of about 11 days, resulting in a bulk TIL population, generally about 1×10⁸ bulk TIL cells.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is melanoma. In some embodiments, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, therapeutically effective dosage is about 2.3×10¹⁰ to about 13.7×10¹⁰. In some embodiments, therapeutically effective dosage is about 7.8×10¹⁰ TILs, particularly of the cancer is melanoma. In some embodiments, therapeutically effective dosage is about 1.2×10¹⁰ to about 4.3×10¹⁰ of TILs. In some embodiments, therapeutically effective dosage is about 3×10¹⁰ to about 12×10¹⁰ TILs. In some embodiments, therapeutically effective dosage is about 4×10¹⁰ to about 10×10¹⁰ TILs. In some embodiments, therapeutically effective dosage is about 5×10¹⁰ to about 8×10¹⁰ TILs. In some embodiments, therapeutically effective dosage is about 6×10¹⁰ to about 8×10¹⁰ TILs. In some embodiments, therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸ 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, expansion of TILs may be performed using a priming first expansion step (for example such as those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include processes referred to as pre-REP or priming REP and which contains feeder cells from Day 0 and/or from culture initiation) as described below and herein, followed by a rapid second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein. In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/mL gentamicin. In some embodiments, the first expansion culture medium comprises 2-mercaptoethanol (also referred to as beta-mercaptoethanol). In some embodiments, the first expansion culture medium (e.g., sometimes referred to as CM1 or the first cell culture medium) comprises 55μ 2-mercaptoethanol.

In some embodiments, there are less than or equal to 240 tumor fragments. In some embodiments, there are less than or equal to 240 tumor fragments placed in less than or equal to 4 containers. In some embodiments, the containers are GREX100 MCS flasks. In some embodiments, less than or equal to 60 tumor fragments are placed in 1 container. In some embodiments, each container comprises less than or equal to 500 mL of media per container. In some embodiments, the media comprises IL-2. In some embodiments, the media comprises 6000 IU/mL of IL-2. In some embodiments, the media comprises antigen-presenting feeder cells (also referred to herein as “antigen-presenting cells”). In some embodiments, the media comprises 2.5×10⁸ antigen-presenting feeder cells per container. In some embodiments, the media comprises OKT-3. In some embodiments, the media comprises 30 ng/mL of OKT-3 per container. In some embodiments, the container is a GREX100 MCS flask. In some embodiments, the media comprises 6000 IU/mL of IL-2, 30 ng of OKT-3, and 2.5×10⁸ antigen-presenting feeder cells. In some embodiments, the media comprises 6000 IU/mL of IL-2, 30 ng/mL of OKT-3, and 2.5×10⁸ antigen-presenting feeder cells per container.

After preparation of the tumor fragments, whole tumor digests, and/or whole tumor cell suspensions, the resulting cells (i.e., fragments and/or digests which is a primary cell population) are cultured in media containing IL-2, antigen-presenting feeder cells and OKT-3 under conditions that favor the growth of TILs over tumor and other cells and which allow for TIL priming and accelerated growth from initiation of the culture on Day 0. In some embodiments, the tumor digests and/or tumor fragments are incubated in with 6000 IU/mL of IL-2, as well as antigen-presenting feeder cells and OKT-3. This primary cell population is cultured for a period of days, generally from 1 to 8 days, resulting in a bulk TIL population, generally about 1×10⁸ bulk TIL cells. In some embodiments, the growth media during the priming first expansion comprises IL-2 or a variant thereof, as well as antigen-presenting feeder cells and OKT-3. In some embodiments, this primary cell population is cultured for a period of days, generally from 1 to 7 days, resulting in a bulk TIL population, generally about 1×10⁸ bulk TIL cells. In some embodiments, the growth media during the priming first expansion comprises IL-2 or a variant thereof, as well as antigen-presenting feeder cells and OKT-3. In some embodiments, the IL-2 is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in Example C. In some embodiments, the priming first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the priming first expansion culture media comprises about 9,000 IU/mL of IL-2 to about 5,000 IU/mL of IL-2. In some embodiments, the priming first expansion culture media comprises about 8,000 IU/mL of IL-2 to about 6,000 IU/mL of IL-2. In some embodiments, the priming first expansion culture media comprises about 7,000 IU/mL of IL-2 to about 6,000 IU/mL of IL-2. In some embodiments, the priming first expansion culture media comprises about 6,000 IU/mL of IL-2. In some embodiments, the cell culture medium further comprises IL-2. In some embodiments, the priming first expansion cell culture medium comprises about 3000 IU/mL of IL-2. In some embodiments, the priming first expansion cell culture medium further comprises IL-2. In some embodiments, the priming first expansion cell culture medium comprises about 3000 IU/mL of IL-2. In some embodiments, the priming first expansion cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the priming first expansion cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

In some embodiments, priming first expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the priming first expansion culture media comprises about 500 IU/mL of IL-15 to about 100 IU/mL of IL-15. In some embodiments, the priming first expansion culture media comprises about 400 IU/mL of IL-15 to about 100 IU/mL of IL-15. In some embodiments, the priming first expansion culture media comprises about 300 IU/mL of IL-15 to about 100 IU/mL of IL-15. In some embodiments, the priming first expansion culture media comprises about 200 IU/mL of IL-15. In some embodiments, the priming first expansion cell culture medium comprises about 180 IU/mL of IL-15. In some embodiments, the priming first expansion cell culture medium further comprises IL-15. In some embodiments, the priming first expansion cell culture medium comprises about 180 IU/mL of IL-15.

In some embodiments, priming first expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the priming first expansion culture media comprises about 20 IU/mL of IL-21 to about 0.5 IU/mL of IL-21. In some embodiments, the priming first expansion culture media comprises about 15 IU/mL of IL-21 to about 0.5 IU/mL of IL-21. In some embodiments, the priming first expansion culture media comprises about 12 IU/mL of IL-21 to about 0.5 IU/mL of IL-21. In some embodiments, the priming first expansion culture media comprises about 10 IU/mL of IL-21 to about 0.5 IU/mL of IL-21. In some embodiments, the priming first expansion culture media comprises about 5 IU/mL of IL-21 to about 1 IU/mL of IL-21. In some embodiments, the priming first expansion culture media comprises about 2 IU/mL of IL-21. In some embodiments, the priming first expansion cell culture medium comprises about 1 IU/mL of IL-21. In some embodiments, the priming first expansion cell culture medium comprises about 0.5 IU/mL of IL-21. In some embodiments, the cell culture medium further comprises IL-21. In some embodiments, the priming first expansion cell culture medium comprises about 1 IU/mL of IL-21.

In some embodiments, the priming first expansion cell culture medium comprises OKT-3 antibody. In some embodiments, the priming first expansion cell culture medium comprises about 30 ng/mL of OKT-3 antibody. In some embodiments, the priming first expansion cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium comprises between 15 ng/ml and 30 ng/mL of OKT-3 antibody. In some embodiments, the cell culture medium comprises 30 ng/mL of OKT-3 antibody. In some embodiments, the OKT-3 antibody is muromonab.

In some embodiments, the priming first expansion cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the priming first expansion cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist. In some embodiments, in addition to one or more TNFRSF agonists, the priming first expansion cell culture medium further comprises IL-2 at an initial concentration of about 6000 IU/mL and OKT-3 antibody at an initial concentration of about 30 ng/mL, and wherein the one or more TNFRSF agonists comprises a 4-1BB agonist.

In some embodiments, the priming first expansion culture medium is referred to as “CM”, an abbreviation for culture media. In some embodiments, it is referred to as CM1 (culture medium 1). In some embodiments, CM consists of RPMI 1640 with GlutaMAX, supplemented with 10% human AB serum, 25 mM Hepes, and 10 mg/mL gentamicin. In some embodiments, the CM is the CM1 described in the Examples, see, Example A. In some embodiments, the priming first expansion occurs in an initial cell culture medium or a first cell culture medium. In some embodiments, the priming first expansion culture medium or the initial cell culture medium or the first cell culture medium comprises IL-2, OKT-3 and antigen-presenting feeder cells (also referred to herein as feeder cells).

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific). In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM. In some embodiments, the final concentration of 2-mercaptoethanol in the media is 55 μM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, One or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, histidine, L-isoleucine, L-methionine, L-phenylalanine, L-hydroxyproline, L-serine, L-threonine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (MEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1X Medium” in Table 4. In other embodiments, the non-trace element moiety ingredients in the defined medium are present in the final concentrations listed in the column under the heading “A Preferred Embodiment of the 1X Medium” in Table 4. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 4.

TABLE 4
Concentrations of Non-Trace Element Moiety Ingredients

| | A preferred | Concentration range | A preferred |
|---|---|---|---|
| | embodiment in | in 1X medium | embodiment in 1X |
| | supplement (mg/L) | (mg/L) | medium (mg/L) |
| Ingredient | (About) | (About) | (About) |
| Glycine | 150 | 5-200 | 53 |
| L-Histidine | 940 | 5-250 | 183 |
| L-Isoleucine | 3400 | 5-300 | 615 |
| L-Methionine | 90 | 5-200 | 44 |
| L-Phenylalanine | 1800 | 5-400 | 336 |
| L-Proline | 4000 | 1-1000 | 600 |
| L-Hydroxyproline | 100 | 1-45 | 15 |
| L-Serine | 800 | 1-250 | 162 |
| L-Threonine | 2200 | 10-500 | 425 |
| L-Tryptophan | 440 | 2-110 | 82 |
| L-Tyrosine | 77 | 3-175 | 84 |
| L-Valine | 2400 | 5-500 | 454 |
| Thiamine | 33 | 1-20 | 9 |
| Reduced Glutathione | 10 | 1-20 | 1.5 |
| Ascorbic Acid-2-PO₄ | 330 | 1-200 | 50 |
| (Mg Salt) | | | |
| Transferrin (iron | 55 | 1-50 | 8 |
| saturated) | | | |
| Insulin | 100 | 1-100 | 10 |
| Sodium Selenite | 0.07 | 0.000001-0.0001 | 0.00001 |
| AlbuMAX ®1 | 83,000 | 5000-50,000 | 12,500 |

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., “Ex vivo expansion of human T cells for adoptive immunotherapy using the novel Xeno-free CTS Immune Cell Serum Replacement,” Clin Transl Immunology, 4 (1) 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or (WE; also known as 2-mercaptoethanol, CAS 60-24-2).

In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 1 to 8 days, as discussed in the examples and figures. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 2 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 3 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 4 to 8 days, as discussed in the examples and figures. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 1 to 7 days, as discussed in the examples and figures. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 2 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 2 to 7 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 3 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 3 to 7 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 4 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 4 to 7 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 5 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 5 to 7 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 6 to 8 days. In some embodiments, the priming first expansion (including processes such as for example those described in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 6 to 7 days. In some embodiments, the priming first expansion (including processes such as for example those provided in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 7 to 8 days. In some embodiments, the priming first expansion (including processes such as for example those provided in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 8 days. In some embodiments, the priming first expansion (including processes such as for example those provided in Step B of FIG. 1 (in particular, e.g., FIG. 1B and/or FIG. 1C and/or FIG. 1D and/or FIG. 1E and/or FIG. 1F and/or FIG. 1G and/or FIG. 1H), which can include those sometimes referred to as the pre-REP or priming REP) process is 7 days.

In some embodiments, the priming first TIL expansion can proceed for 1 days to 8 days from when fragmentation occurs and/or when the first priming expansion step is initiated. In some embodiments, the priming first TIL expansion can proceed for 1 days to 7 days from when fragmentation occurs and/or when the first priming expansion step is initiated. In some embodiments, the priming first TIL expansion can proceed for 2 days to 8 days from when fragmentation occurs and/or when the first priming expansion step is initiated. In some embodiments, the priming first TIL expansion can proceed for 2 days to 7 days from when fragmentation occurs and/or when the first priming expansion step is initiated. In some embodiments, the priming first TIL expansion can proceed for 3 days to 8 days from when fragmentation occurs and/or when the first priming expansion step is initiated. In some embodiments, the priming first TIL expansion can proceed for 3 days to 7 days from when fragmentation occurs and/or when the first priming expansion step is initiated. In some embodiments, the priming first TIL expansion can proceed for 4 days to 8 days from when fragmentation occurs and/or when the first priming expansion step is initiated. In some embodiments, the priming first TIL expansion can proceed for 4 days to 7 days from when fragmentation occurs and/or when the first priming expansion step is initiated. In some embodiments, the priming first TIL expansion can proceed for 5 days to 8 days from when fragmentation occurs and/or when the first priming expansion step is initiated. In some embodiments, the priming first TIL expansion can proceed for 5 days to 7 days from when fragmentation occurs and/or when the first priming expansion step is initiated. In some embodiments, the priming first TIL expansion can proceed for 6 days to 8 days from when fragmentation occurs and/or when the first priming expansion step is initiated. In some embodiments, the priming first TIL expansion can proceed for 6 days to 7 days from when fragmentation occurs and/or when the first priming expansion step is initiated. In some embodiments, the priming first TIL expansion can proceed for 7 to 8 days from when fragmentation occurs and/or when the first priming expansion step is initiated. In some embodiments, the priming first TIL expansion can proceed for 8 days from when fragmentation occurs and/or when the first priming expansion step is initiated. In some embodiments, the priming first TIL expansion can proceed for 7 days from when fragmentation occurs and/or when the first priming expansion step is initiated.

In some embodiments, the priming first expansion of the TILs can proceed for 1 day, 2 days, 3 days, 4 days, 5 days, 6 days, 7 days, 8 days, 9 days, 10 days, or 11 days. In some embodiments, the first TIL expansion can proceed for 1 day to 8 days. In some embodiments, the first TIL expansion can proceed for 1 day to 7 days. In some embodiments, the first TIL expansion can proceed for 2 days to 7 days. In some embodiments, the first TIL expansion can proceed for 3 days to 7 days. In some embodiments, the first TIL expansion can proceed for 4 days to 7 days. In some embodiments, the first TIL expansion can proceed for 5 days to 7 days. In some embodiments, the first TIL expansion can proceed for 6 days to 7 days. In some embodiments, the first TIL expansion can proceed for 2 days to 8 days. In some embodiments, the first TIL expansion can proceed for 3 days to 8 days. In some embodiments, the first TIL expansion can proceed for 4 days to 8 days. In some embodiments, the first TIL expansion can proceed for 5 days to 8 days. In some embodiments, the first TIL expansion can proceed for 6 days to 8 days. In some embodiments, the first TIL expansion can proceed for 2 days to 9 days. In some embodiments, the first TIL expansion can proceed for 3 days to 9 days. In some embodiments, the first TIL expansion can proceed for 4 days to 9 days. In some embodiments, the first TIL expansion can proceed for 5 days to 9 days. In some embodiments, the first TIL expansion can proceed for 6 days to 9 days. In some embodiments, the first TIL expansion can proceed for 2 days to 10 days. In some embodiments, the first TIL expansion can proceed for 3 days to 10 days. In some embodiments, the first TIL expansion can proceed for 4 days to 10 days. In some embodiments, the first TIL expansion can proceed for 5 days to 10 days. In some embodiments, the first TIL expansion can proceed for 6 days to 10 days. In some embodiments, the first TIL expansion can proceed for 2 days to 11 days. In some embodiments, the first TIL expansion can proceed for 3 days to 11 days. In some embodiments, the first TIL expansion can proceed for 4 days to 11 days. In some embodiments, the first TIL expansion can proceed for 5 days to 11 days. In some embodiments, the first TIL expansion can proceed for 6 days to 11 days. In some embodiments, the first TIL expansion can proceed for 7 days. In some embodiments, the first TIL expansion can proceed for 8 days. In some embodiments, the first TIL expansion can proceed for 9 days. In some embodiments, the first TIL expansion can proceed for 10 days. In some embodiments, the first TIL expansion can proceed for 11 days.

In some embodiments, a combination of IL-2, IL-7, IL-15, and/or IL-21 are employed as a combination during the priming first expansion. In some embodiments, IL-2, IL-7, IL-15, and/or IL-21 as well as any combinations thereof can be included during the priming first expansion, including for example during a Step B processes according to FIG. 1 (in particular, e.g., FIG. 1B), as well as described herein. In some embodiments, a combination of IL-2, IL-15, and IL-21 are employed as a combination during the priming first expansion. In some embodiments, IL-2, IL-15, and IL-21 as well as any combinations thereof can be included during Step B processes according to FIG. 1 (in particular, e.g., FIG. 1B and/or FIG. 1C and/or FIG. 1D and/or FIG. 1E and/or FIG. 1F and/or FIG. 1G and/or FIG. 1H) and as described herein.

In some embodiments, the priming first expansion, for example, Step B according to FIG. 1 (in particular, e.g., FIG. 1B and/or FIG. 1C and/or FIG. 1D and/or FIG. 1E and/or FIG. 1F and/or FIG. 1G and/or FIG. 1H), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-10 or a G-REX-100. In some embodiments, the bioreactor employed is a G-REX-100. In some embodiments, the bioreactor employed is a G-REX-10.

1. Feeder Cells and Antigen Presenting Cells

In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion (priming REP). In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during days 4-8. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during days 4-7. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during days 5-8. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during days 5-7. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during days 6-8. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during days 6-7. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during day 7 or 8. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during day 7. In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) does not require feeder cells (also referred to herein as “antigen-presenting cells”) at the initiation of the TIL expansion, but rather are added during the priming first expansion at any time during day 8.

In some embodiments, the priming first expansion procedures described herein (for example including expansion such as those described in Step B from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as pre-REP or priming REP) require feeder cells (also referred to herein as “antigen-presenting cells”) at the initiation of the TIL expansion and during the priming first expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, 2.5×10⁸ feeder cells are used during the priming first expansion. In some embodiments, 2.5×10⁸ feeder cells per container are used during the priming first expansion. In some embodiments, 2.5×10⁸ feeder cells per GREX-10 are used during the priming first expansion. In some embodiments, 2.5×10⁸ feeder cells per GREX-100 are used during the priming first expansion.

In general, the allogenic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and acceptable for use in the TIL expansion procedures described herein if the total number of viable cells on day 14 is less than the initial viable cell number put into culture on day 0 of the priming first expansion.

In some embodiments, PBMCs are considered replication incompetent and acceptable for use in the TIL expansion procedures described herein if the total number of viable cells, cultured in the presence of OKT3 and IL-2, on day 7 have not increased from the initial viable cell number put into culture on day 0 of the priming first expansion. In some embodiments, the PBMCs are cultured in the presence of 30 ng/mL OKT3 antibody and 3000 IU/mL IL-2. In some embodiments, the PBMCs are cultured in the presence of 30 ng/ml OKT3 antibody and 6000 IU/ml IL-2.

In some embodiments, PBMCs are considered replication incompetent and acceptable for use in the TIL expansion procedures described herein if the total number of viable cells, cultured in the presence of OKT3 and IL-2, on day 7 have not increased from the initial viable cell number put into culture on day 0 of the priming first expansion. In some embodiments, the PBMCs are cultured in the presence of 5-60 ng/mL OKT3 antibody and 1000-6000 IU/mL IL-2. In some embodiments, the PBMCs are cultured in the presence of 10-50 ng/ml OKT3 antibody and 2000-5000 IU/mL IL-2. In some embodiments, the PBMCs are cultured in the presence of 20-40 ng/ml OKT3 antibody and 2000-4000 IU/mL IL-2. In some embodiments, the PBMCs are cultured in the presence of 25-35 ng/ml OKT3 antibody and 2500-3500 IU/mL IL-2. In some embodiments, the PBMCs are cultured in the presence of 30 ng/ml OKT3 antibody and 6000 IU/mL IL-2. In some embodiments, the PBMCs are cultured in the presence of 15 ng/ml OKT3 antibody and 3000 IU/mL IL-2. In some embodiments, the PBMCs are cultured in the presence of 15 ng/mL OKT3 antibody and 6000 IU/ml IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the priming first expansion procedures described herein require a ratio of about 2.5×10⁸ feeder cells to about 100×10⁶ TILs. In other embodiments, the priming first expansion procedures described herein require a ratio of about 2.5×10⁸ feeder cells to about 50×10⁶ TILs. In yet another embodiment, the priming first expansion described herein require about 2.5×10⁸ feeder cells to about 25×10⁶ TILs. In yet another embodiment, the priming first expansion described herein require about 2.5×10⁸ feeder cells. In yet another embodiment, the priming first expansion requires one-fourth, one-third, five-twelfths, or one-half of the number of feeder cells used in the rapid second expansion.

In some embodiments, the media in the priming first expansion comprises IL-2. In some embodiments, the media in the priming first expansion comprises 6000 IU/mL of IL-2. In some embodiments, the media in the priming first expansion comprises antigen-presenting feeder cells. In some embodiments, the media in the priming first expansion comprises 25×10⁸ antigen-presenting feeder cells per container. In some embodiments, the media in the priming first expansion comprises OKT-3. In some embodiments, the media comprises 30 ng of OKT-3 per container. In some embodiments, the container is a GREX100 MCS flask. In some embodiments, the media comprises 6000 IU/mL of IL-2, 30 ng/mL of OKT-3, and 2.5×10⁸ antigen-presenting feeder cells. In some embodiments, the media comprises 6000 IU/mL of IL-2, 30 ng/mL of OKT-3, and 2.5×10⁸ antigen-presenting feeder cells per container. In some embodiments, the media comprises 500 mL of culture medium and 15 pg of OKT-3 per 2.5×10⁸ antigen-presenting feeder cells per container. In some embodiments, the media comprises 500 mL of culture medium and 15 pg of OKT-3 per container. In some embodiments, the container is a GREX100 MCS flask. In some embodiments, the media comprises 500 mL of culture medium and 6000 IU/mL of IL-2, 30 ng/mL of OKT-3, and 2.5×10⁸ antigen-presenting feeder cells. In some embodiments, the media comprises 500 mL of culture medium and 6000 IU/mL of IL-2, 15 μg of OKT-3, and 2.5×10⁸ antigen-presenting feeder cells per container. In some embodiments, the media comprises 500 mL of culture medium and 15 μg of OKT-3 per 2.5×10⁸ antigen-presenting feeder cells per container.

In some embodiments, the priming first expansion procedures described herein require an excess of feeder cells over TILs during the second expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, artificial antigen-presenting (aAPC) cells are used in place of PBMCs.

In general, the allogenic PBMCs are inactivated, either via irradiation or heat treatment, and used in the TIL expansion procedures described herein, including the exemplary procedures described in the figures and examples.

In some embodiments, artificial antigen presenting cells are used in the priming first expansion as a replacement for, or in combination with, PBMCs.

2. Cytokines

The expansion methods described herein generally use culture media with high doses of a cytokine, in particular IL-2, as is known in the art.

Alternatively, using combinations of cytokines for the priming first expansion of TILs is additionally possible, with combinations of two or more of IL-2, IL-15 and IL-21 as is generally outlined in International Publication No. WO 2015/189356 and WO 2015/189357, hereby expressly incorporated by reference in their entirety. Thus, possible combinations include IL-2 and IL-15, IL-2 and IL-21, IL-15 and IL-21, and IL-2, IL-15 and IL-21, with the latter finding particular use in many embodiments. The use of combinations of cytokines specifically favors the generation of lymphocytes, and in particular T-cells as described therein.

TABLE 5
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH IRFRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | MHKCDITLQE IIKTLNSLTE CKILCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 6 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL IKVSEGTTIL LNCTGQVKGR KPAALGEAQF TKSLEENKSL | 120 |
| human IL-7 | KEQKKLNDLC FLKRLLQEIK TCWNKILMGT KEH | 153 |
| (rhIL-7) | | |
| SEQ ID NO: 7 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 8 | MQDRHMIRMR QLIDIVDQLK NYVNDLVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | HLSSRTHGSE DS | 132 |
| (rhIL-21) | | |

C. STEP C: Priming First Expansion to Rapid Second Expansion Transition

In some cases, the bulk TIL population obtained from the priming first expansion (which can include expansions sometimes referred to as pre-REP), including, for example, the TIL population obtained from for example, Step B as indicated in FIG. 1 (in particular, e.g., FIG. 1B and/or FIG. 1C and/or FIG. 1D and/or FIG. 1E and/or FIG. 1F and/or FIG. 1G and/or FIG. 1H), can be subjected to a rapid second expansion (which can include expansions sometimes referred to as Rapid Expansion Protocol (REP)) and then cryopreserved as discussed below. Similarly, in the case where genetically modified TILs will be used in therapy, the expanded TIL population from the priming first expansion or the expanded TIL population from the rapid second expansion can be subjected to genetic modifications for suitable treatments prior to the expansion step or after the priming first expansion and prior to the rapid second expansion.

In some embodiments, the TILs obtained from the priming first expansion (for example, from Step B as indicated in FIG. 1 (in particular, e.g., FIG. 1B and/or FIG. 1C and/or FIG. 1D and/or FIG. 1E and/or FIG. 1F and/or FIG. 1G and/or FIG. 1H)) are stored until phenotyped for selection. In some embodiments, the TILs obtained from the priming first expansion (for example, from Step B as indicated in FIG. 1 (in particular, e.g., FIG. 1B and/or FIG. 1C and/or FIG. 1D and/or FIG. 1E and/or FIG. 1F and/or FIG. 1G and/or FIG. 1H)) are not stored and proceed directly to the rapid second expansion. In some embodiments, the TILs obtained from the priming first expansion are not cryopreserved after the priming first expansion and prior to the rapid second expansion. In some embodiments, the transition from the priming first expansion to the second expansion occurs at about 2 days, 3 days, 4 days, 5 days, 6 days, 7 days, or 8 days from when tumor fragmentation occurs and/or when the first priming expansion step is initiated. In some embodiments, the transition from the priming first expansion to the rapid second expansion occurs at about 3 days to 7 days from when fragmentation occurs and/or when the first priming expansion step is initiated. In some embodiments, the transition from the priming first expansion to the rapid second expansion occurs at about 3 days to 8 days from when fragmentation occurs and/or when the first priming expansion step is initiated. In some embodiments, the transition from the priming first expansion to the second expansion occurs at about 4 days to 7 days from when fragmentation occurs and/or when the first priming expansion step is initiated. In some embodiments, the transition from the priming first expansion to the second expansion occurs at about 4 days to 8 days from when fragmentation occurs and/or when the first priming expansion step is initiated. In some embodiments, the transition from the priming first expansion to the second expansion occurs at about 5 days to 7 days from when fragmentation occurs and/or when the first priming expansion step is initiated. In some embodiments, the transition from the priming first expansion to the second expansion occurs at about 5 days to 8 days from when fragmentation occurs and/or when the first priming expansion step is initiated. In some embodiments, the transition from the priming first expansion to the second expansion occurs at about 6 days to 7 days from when fragmentation occurs and/or when the first priming expansion step is initiated. In some embodiments, the transition from the priming first expansion to the second expansion occurs at about 6 days to 8 days from when fragmentation occurs and/or when the first priming expansion step is initiated. In some embodiments, the transition from the priming first expansion to the second expansion occurs at about 7 days to 8 days from when fragmentation occurs and/or when the first priming expansion step is initiated. In some embodiments, the transition from the priming first expansion to the second expansion occurs at about 7 days from when fragmentation occurs and/or when the first priming expansion step is initiated. In some embodiments, the transition from the priming first expansion to the second expansion occurs at about 8 days from when fragmentation occurs and/or when the first priming expansion step is initiated.

In some embodiments, the transition from the priming first expansion to the rapid second expansion occurs at 1 day, 2 days, 3 days, 4 days, 5 days, 6 days, 7 days, or 8 days from when fragmentation occurs and/or when the first priming expansion step is initiated. In some embodiments, the transition from the priming first expansion to the rapid second expansion occurs 1 day to 7 days from when fragmentation occurs and/or when the first priming expansion step is initiated. In some embodiments, the transition from the priming first expansion to the rapid second expansion occurs 1 day to 8 days from when fragmentation occurs and/or when the first priming expansion step is initiated. In some embodiments, the transition from the priming first expansion to the second expansion occurs 2 days to 7 days from when fragmentation occurs and/or when the first priming expansion step is initiated. In some embodiments, the transition from the priming first expansion to the second expansion occurs 2 days to 8 days from when fragmentation occurs and/or when the first priming expansion step is initiated. In some embodiments, the transition from the priming first expansion to the second expansion occurs 3 days to 7 days from when fragmentation occurs and/or when the first priming expansion step is initiated. In some embodiments, the transition from the priming first expansion to the second expansion occurs 3 days to 8 days from when fragmentation occurs and/or when the first priming expansion step is initiated. In some embodiments, the transition from the priming first expansion to the rapid second expansion occurs 4 days to 7 days from when fragmentation occurs and/or when the first priming expansion step is initiated. In some embodiments, the transition from the priming first expansion to the rapid second expansion occurs 4 days to 8 days from when fragmentation occurs and/or when the first priming expansion step is initiated. In some embodiments, the transition from the priming first expansion to the rapid second expansion occurs 5 days to 7 days from when fragmentation occurs and/or when the first priming expansion step is initiated. In some embodiments, the transition from the priming first expansion to the rapid second expansion occurs 5 days to 8 days from when fragmentation occurs and/or when the first priming expansion step is initiated. In some embodiments, the transition from the priming first expansion to the rapid second expansion occurs 6 days to 7 days from when fragmentation occurs and/or when the first priming expansion step is initiated. In some embodiments, the transition from the priming first expansion to the rapid second expansion occurs 6 days to 8 days from when fragmentation occurs and/or when the first priming expansion step is initiated. In some embodiments, the transition from the priming first expansion to the rapid second expansion occurs 7 days to 8 days from when fragmentation occurs and/or when the first priming expansion step is initiated. In some embodiments, the transition from the priming first expansion to the rapid second expansion occurs 7 days from when fragmentation occurs and/or when the first priming expansion step is initiated. In some embodiments, the transition from the priming first expansion to the rapid second expansion occurs 8 days from when fragmentation occurs and/or when the first priming expansion step is initiated.

In some embodiments, the TILs are not stored after the primary first expansion and prior to the rapid second expansion, and the TILs proceed directly to the rapid second expansion (for example, in some embodiments, there is no storage during the transition from Step B to Step D as shown in FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments, the transition occurs in closed system, as described herein. In some embodiments, the TILs from the priming first expansion, the second population of TILs, proceeds directly into the rapid second expansion with no transition period.

In some embodiments, the transition from the priming first expansion to the rapid second expansion, for example, Step C according to FIG. 1 (in particular, e.g., FIG. 1B), is performed in a closed system bioreactor. In some embodiments, a closed system is employed for the TIL expansion, as described herein. In some embodiments, a single bioreactor is employed. In some embodiments, the single bioreactor employed is for example a GREX-10 or a GREX-100. In some embodiments, the closed system bioreactor is a single bioreactor. In some embodiments, the transition from the priming first expansion to the rapid second expansion involves a scale-up in container size. In some embodiments, the priming first expansion is performed in a smaller container than the rapid second expansion. In some embodiments, the priming first expansion is performed in a GREX-100 and the rapid second expansion is performed in a GREX-500.

In some embodiments, a maximum of 1×10⁶ cells TILs are obtained at the end of the priming first expansion. In some embodiments, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, or 0.5×10⁶ TILs are obtained at the end of the priming first expansion. In some embodiments, the TILs at the end of the priming first expansion are about 9% to about 40% PD-1+. In some embodiments, the TILs at the end of the priming first expansion are about 10% to about 40% PD-1+. In some embodiments, the TILs at the end of the priming first expansion are about 15% to about 30% PD-1+. In some embodiments, the TILs at the end of the priming first expansion are about 20% to about 40% PD-1+. In some embodiments, the TILs at the end of the priming first expansion are about 20% to about 30% PD-1+. In some embodiments, the TILs at the end of the priming first expansion are about 10% to about 20% PD-1+. In some embodiments, the TILs at the end of the priming first expansion are about 9%, about 10%, about 15%, about 20%, about 25%, about 30%, about 35%; or about 40% PD-1+. In some embodiments, the TILs at the end of the priming first expansion are about 9% to about 40% PD-1high. In some embodiments, the TILs at the end of the priming first expansion are about 15% to about 30% PD-1high. In some embodiments, the TILs at the end of the priming first expansion are about 20% to about 40% PD-1high. In some embodiments, the TILs at the end of the priming first expansion are about 20% to about 30% PD-1high. In some embodiments, the TILs at the end of the priming first expansion are about 10% to about 20% PD-1high. In some embodiments, the TILs at the end of the priming first expansion are about 9%, about 10%, about 15%, about 20%, about 25%, about 30%, about 35%, or about 40% PD-1high.

D. STEP D: Rapid Second Expansion

In some embodiments, the TIL cell population is further expanded in number after harvest and the priming first expansion, after Step A and Step B. and the transition referred to as Step C, as indicated in FIG. 1 (in particular, e.g., FIG. 1B and/or FIG. 1C and/or FIG. 1D and/or FIG. 1E and/or FIG. 1F and/or FIG. 1G and/or FIG. 1H). This further expansion is referred to herein as the rapid second expansion, which can include expansion processes generally referred to in the art as a rapid expansion process (Rapid Expansion Protocol or REP; as well as processes as indicated in Step D of FIG. 1 (in particular, e.g., FIG. 1B and/or FIG. 1C and/or FIG. 1D and/or FIG. 1E and/or FIG. 1F and/or FIG. 1G and/or FIG. 1H). The rapid second expansion is generally accomplished using a culture media comprising a number of components, including feeder cells, a cytokine source, and an anti-CD3 antibody, in a gas-permeable container. In some embodiments, 1 day, 2 days, 3 days, or 4 days after initiation of the rapid second expansion (i.e., at days 8, 9, 10, or 11 of the overall Gen 3 process), the TILs are transferred to a larger volume container. In some embodiments, this rapid second expansion is referred to as activation II.

In some embodiments, a maximum of 1×10⁶ cells TILs are added at the beginning of the rapid second expansion. In some embodiments, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, or 0.5×10⁶ TILs are added at the beginning of the rapid second expansion. In some embodiments, the maximum cell density from the priming first expansion is 1e6 cells to provide 1e9 for initiating the rapid second expansion.

In some embodiments, the rapid second expansion (which can include expansions sometimes referred to as REP; as well as processes as indicated in Step D of FIG. 1 (in particular, e.g., FIG. 1B and/or FIG. 1C and/or FIG. 1D and/or FIG. 1E and/or FIG. 1F and/or FIG. 1G and/or FIG. 1H) of TIL can be performed using any TIL flasks or containers known by those of skill in the art. In some embodiments, the second TIL expansion can proceed for 1 day, 2 days, 3 days, 4 days, 5 days, 6 days, 7 days, 8 days, 9 days or 10 days after initiation of the rapid second expansion. In some embodiments, the second TIL expansion can proceed for about 1 days to about 9 days after initiation of the rapid second expansion. In some embodiments, the second TIL expansion can proceed for about 1 days to about 10 days after initiation of the rapid second expansion. In some embodiments, the second TIL expansion can proceed for about 2 days to about 9 days after initiation of the rapid second expansion. In some embodiments, the second TIL expansion can proceed for about 2 days to about 10 days after initiation of the rapid second expansion. In some embodiments, the second TIL expansion can proceed for about 3 days to about 9 days after initiation of the rapid second expansion. In some embodiments, the second TIL expansion can proceed for about 3 days to about 10 days after initiation of the rapid second expansion. In some embodiments, the second TIL expansion can proceed for about 4 days to about 9 days after initiation of the rapid second expansion. In some embodiments, the second TIL expansion can proceed for about 4 days to about 10 days after initiation of the rapid second expansion. In some embodiments, the second TIL expansion can proceed for about 5 days to about 9 days after initiation of the rapid second expansion. In some embodiments, the second TIL expansion can proceed for about 5 days to about 10 days after initiation of the rapid second expansion. In some embodiments, the second TIL expansion can proceed for about 6 days to about 9 days after initiation of the rapid second expansion. In some embodiments, the second TIL expansion can proceed for about 6 days to about 10 days after initiation of the rapid second expansion. In some embodiments, the second TIL expansion can proceed for about 7 days to about 9 days after initiation of the rapid second expansion. In some embodiments, the second TIL expansion can proceed for about 7 days to about 10 days after initiation of the rapid second expansion. In some embodiments, the second TIL expansion can proceed for about 8 days to about 9 days after initiation of the rapid second expansion. In some embodiments, the second TIL expansion can proceed for about 8 days to about 10 days after initiation of the rapid second expansion. In some embodiments, the second TIL expansion can proceed for about 9 days to about 10 days after initiation of the rapid second expansion. In some embodiments, the second TIL expansion can proceed for about 1 day after initiation of the rapid second expansion. In some embodiments, the second TIL expansion can proceed for about 2 days after initiation of the rapid second expansion. In some embodiments, the second TIL expansion can proceed for about 3 days after initiation of the rapid second expansion. In some embodiments, the second TIL expansion can proceed for about 4 days after initiation of the rapid second expansion. In some embodiments, the second TIL expansion can proceed for about 5 days after initiation of the rapid second expansion. In some embodiments, the second TIL expansion can proceed for about 6 days after initiation of the rapid second expansion. In some embodiments, the second TIL expansion can proceed for about 7 days after initiation of the rapid second expansion. In some embodiments, the second TIL expansion can proceed for about 8 days after initiation of the rapid second expansion. In some embodiments, the second TIL expansion can proceed for about 9 days after initiation of the rapid second expansion. In some embodiments, the second TIL expansion can proceed for about 10 days after initiation of the rapid second expansion.

In some embodiments, the rapid second expansion occurs as two periods, comprising an activation II period followed by a split or division and further growth period within the rapid second expansion. In some embodiments, the rapid second expansion occurs for a period of 1 to 11 days. In some embodiments, rapid second expansion occurs for a period of 1 to 10 days, resulting in a bulk TIL population. In some embodiments, the rapid second expansion occurs for a period of 1 to 9 days. In some embodiments, rapid second expansion occurs for a period of 1 to 8 days. In some embodiments, rapid second expansion comprises an activation II period of 1 to 4 days followed by a split or division and further growth period of 1 to 7 days. In some embodiments, rapid second expansion comprises an activation II period of 1 to 3 days followed by a split or division and further growth period of 1 to 6 days. In some embodiments, rapid second expansion comprises an activation II period of 1 to 4 days followed by a split and further growth period of 1 to 6 days. In some embodiments, rapid second expansion comprises an activation II period of 1 to 7 days followed by a split or division and further growth period of 1 to 7 days. In some embodiments, rapid second expansion comprises an activation II period of 1 to 3 days followed by a split or division and further growth period of 1 to 7 days. In some embodiments, rapid second expansion comprises an activation II period of 1 day, 2 days, 3 days, or 4 days followed by a split or division and further growth period of 1 day, 2 days, 3 days, 4 days, 5 days, 6 days, or 7 days. In some embodiments, the split or division can also include a scale-up to an increased number of containers (including, for example, bags and/or GREX containers). In some embodiments, the split or division can also include a scale-up to an increased number of containers (including, for example, bags and/or GREX containers) from the number of containers during the activation II step to the increased number of containers during the further growth period.

In some embodiments, the rapid second expansion can be performed in a gas permeable container using the methods of the present disclosure (including for example, expansions referred to as REP; as well as processes as indicated in Step D of FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments, the TILs are expanded in the rapid second expansion in the presence of IL-2, OKT-3, and feeder cells (also referred herein as “antigen-presenting cells”). In some embodiments, the TILs are expanded in the rapid second expansion in the presence of IL-2, OKT-3, and feeder cells, wherein the feeder cells are added to a final concentration that is twice, 2.4 times, 2.5 times, 3 times, 3.5 times or 4 times the concentration of feeder cells present in the priming first expansion. For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/ml of OKT3, a mouse monoclonal anti-CD3 antibody (commercially available from Ortho-McNeil, Raritan, NJ or Miltenyi Biotech, Auburn, Calif.) or UHCT-1 (commercially available from BioLegend, San Diego, Calif., USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In some embodiments, the cell culture medium comprises OKT-3 antibody. In some embodiments, the cell culture medium comprises about 30 ng/mL of OKT-3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium comprises between 30 ng/ml and 60 ng/mL of OKT-3 antibody. In some embodiments, the cell culture medium comprises about 60 ng/mL OKT-3. In some embodiments, the OKT-3 antibody is muromonab.

In some embodiments, the media in the rapid second expansion comprises IL-2. In some embodiments, the media comprises 6000 IU/mL of IL-2. In some embodiments, the media in the rapid second expansion comprises antigen-presenting feeder cells. In some embodiments, the media in the rapid second expansion comprises 7.5×10⁸ antigen-presenting feeder cells per container. In some embodiments, the media in the rapid second expansion comprises OKT-3. In some embodiments, the in the rapid second expansion media comprises 500 mL of culture medium and 30 lug of OKT-3 per container. In some embodiments, the container is a GREX100 MCS flask. In some embodiments, the in the rapid second expansion media comprises 6000 IU/mL of IL-2, 60 ng/mL of OKT-3, and 7.5×10⁸ antigen-presenting feeder cells. In some embodiments, the media comprises 500 mL of culture medium and 6000 IU/mL of IL-2, 30 μg of OKT-3, and 7.5×10⁸ antigen-presenting feeder cells per container.

In some embodiments, the media in the rapid second expansion comprises IL-2. In some embodiments, the media comprises 6000 IU/mL of IL-2. In some embodiments, the media in the rapid second expansion comprises antigen-presenting feeder cells. In some embodiments, the media comprises between 5×10⁸ and 7.5×10⁸ antigen-presenting feeder cells per container. In some embodiments, the media in the rapid second expansion comprises OKT-3. In some embodiments, the media in the rapid second expansion comprises 500 mL of culture medium and 30 μg of OKT-3 per container. In some embodiments, the container is a GREX100 MCS flask. In some embodiments, the media in the rapid second expansion comprises 6000 IU/mL of IL-2, 60 ng/mL of OKT-3, and between 5×10⁸ and 7.5×10⁸ antigen-presenting feeder cells. In some embodiments, the media in the rapid second expansion comprises 500 mL of culture medium and 6000 IU/mL of IL-2, 30 jig of OKT-3, and between 5×10⁸ and 7.5×10⁸ antigen-presenting feeder cells per container.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 mg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, a combination of IL-2, IL-7, IL-15, and/or IL-21 are employed as a combination during the second expansion. In some embodiments, IL-2, IL-7, IL-15, and/or IL-21 as well as any combinations thereof can be included during the second expansion, including for example during a Step D processes according to FIG. 1 (in particular, e.g., FIG. 1B and/or FIG. 1C and/or FIG. 1D and/or FIG. 1E and/or FIG. 1F and/or FIG. 1G and/or FIG. 1H), as well as described herein. In some embodiments, a combination of IL-2, IL-15, and IL-21 are employed as a combination during the second expansion. In some embodiments, IL-2, IL-15, and IL-21 as well as any combinations thereof can be included during Step D processes according to FIG. 1 (in particular, e.g., FIG. 1B and/or FIG. 1C and/or FIG. 1D and/or FIG. 1E and/or FIG. 1F and/or FIG. 1G and/or FIG. 1H) and as described herein.

In some embodiments, the second expansion can be conducted in a supplemented cell culture medium comprising IL-2, OKT-3, antigen-presenting feeder cells, and optionally a TNFRSF agonist. In some embodiments, the second expansion occurs in a supplemented cell culture medium. In some embodiments, the supplemented cell culture medium comprises IL-2, OKT-3, and antigen-presenting feeder cells. In some embodiments, the second cell culture medium comprises IL-2, OKT-3, and antigen-presenting cells (APCs; also referred to as antigen-presenting feeder cells). In some embodiments, the second expansion occurs in a cell culture medium comprising IL-2; OKT-3, and antigen-presenting feeder cells (i.e., antigen presenting cells).

In some embodiments, the second expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the second expansion culture media comprises about 500 IU/mL of IL-15 to about 100 IU/mL of IL-15. In some embodiments, the second expansion culture media comprises about 400 IU/mL of IL-15 to about 100 IU/mL of IL-15. In some embodiments, the second expansion culture media comprises about 300 IU/mL of IL-15 to about 100 IU/mL of IL-15. In some embodiments, the second expansion culture media comprises about 200 IU/mL of IL-15. In some embodiments, the cell culture medium comprises about 180 IU/mL of IL-15. In some embodiments, the cell culture medium further comprises IL-15. In some embodiments, the cell culture medium comprises about 180 IU/mL of IL-15.

In some embodiments, the second expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the second expansion culture media comprises about 20 IU/mL of IL-21 to about 0.5 IU/mL of IL-21. In some embodiments, the second expansion culture media comprises about 15 IU/mL of IL-21 to about 0.5 IU/mL of IL-21. In some embodiments, the second expansion culture media comprises about 12 IU/mL of IL-21 to about 0.5 IU/mL of IL-21. In some embodiments, the second expansion culture media comprises about 10 IU/mL of IL-21 to about 0.5 IU/mL of IL-21. In some embodiments, the second expansion culture media comprises about 5 IU/mL of IL-21 to about 1 IU/mL of IL-21. In some embodiments, the second expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In some embodiments, the cell culture medium further comprises IL-21. In some embodiments, the cell culture medium comprises about 1 IU/mL of IL-21.

In some embodiments, the antigen-presenting feeder cells (APCs) are PBMCs. In some embodiments, the ratio of TILs to PBMCs and/or antigen-presenting cells in the rapid expansion and/or the second expansion is about 1 to 10, about 1 to 15, about 1 to 20, about 1 to 25, about 1 to 30, about 1 to 35, about 1 to 40, about 1 to 45, about 1 to 50, about 1 to 75, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to PBMCs in the rapid expansion and/or the second expansion is between 1 to 50 and 1 to 300. In some embodiments, the ratio of TILs to PBMCs in the rapid expansion and/or the second expansion is between 1 to 100 and 1 to 200.

In some embodiments, REP and/or the rapid second expansion is performed in flasks with the bulk TILs being mixed with a 100- or 200-fold excess of inactivated feeder cells, wherein the feeder cell concentration is at least 1.1 times (1.1×), 1.2×, 1.3×, 1.4×, 1.5×, 1.6×, 1.7×, 1.8×, 1.8×, 2×, 2.1×2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9× or 4.0× the feeder cell concentration in the priming first expansion, 30 ng/mL OKT3 anti-CD3 antibody and 6000 IU/mL IL-2 in 150 ml media. Media replacement is done (generally ⅔ media replacement via aspiration of ⅔ of spent media and replacement with an equal volume of fresh media) until the cells are transferred to an alternative growth chamber. Alternative growth chambers include G-REX flasks and gas permeable containers as more fully discussed below.

In some embodiments, the rapid second expansion (which can include processes referred to as the REP process) is 7 to 9 days, as discussed in the examples and figures. In some embodiments, the second expansion is 7 days. In some embodiments, the second expansion is 8 days. In some embodiments, the second expansion is 9 days.

In some embodiments, the second expansion (which can include expansions referred to as REP, as well as those referred to in Step D of FIG. 1 (in particular, e.g., FIG. 1B and/or FIG. 1C and/or FIG. 1D and/or FIG. 1E and/or FIG. 1F and/or FIG. 1G and/or FIG. 1H) may be performed in 500 mL capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 60 ng per ml of anti-CD3 (OKT3). The G-Rex 100 flasks may be incubated at 37° C. in 5% CO₂. On day 5, 250 mL of supernatant may be removed and placed into centrifuge bottles and centrifuged at 1500 rpm (491× g) for 10 minutes. The TIL pellets may be re-suspended with 150 mL of fresh medium with 5% human AB serum, 6000 IU per mL of IL-2, and added back to the original GREX-100 flasks. When TIL are expanded serially in GREX-100 flasks, on day 10 or 11 the TILs can be moved to a larger flask, such as a GREX-500. The cells may be harvested on day 14 of culture. The cells may be harvested on day 15 of culture. The cells may be harvested on day 16 of culture. In some embodiments, media replacement is done until the cells are transferred to an alternative growth chamber. In some embodiments, ⅔ of the media is replaced by aspiration of ⅔ of spent media and replacement with an equal volume of fresh media. In some embodiments, alternative growth chambers include GREX flasks and gas permeable containers as more fully discussed below.

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium OpTimizer™ T-Cell Expansion SFM, CTS™ AIM-V Medium, CTS™ SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Cr³⁺, Ge⁴⁺, Se⁴⁺, Br, T, Mn²⁺, P, si⁴⁺, v⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM. In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins Cir transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1X Medium” in Table 4. In other embodiments, the non-trace element moiety ingredients in the defined medium are present in the final concentrations listed in the column under the heading “A Preferred Embodiment of the 1X Medium” in Table 4. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 4.

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., “Ex vivo expansion of human T cells for adoptive immunotherapy using the novel Xeno-free CTS Immune Cell Serum Replacement,” Clin Transl Immunology, 4 (1) 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or βME; also known as 2-mercaptoethanol, CAS 60-24-2).

In some embodiments, the cell culture media is supplemented with a further cell culture media between days 9 and 17 of the rapid second expansion (including expansions referred to as REP). In some embodiments, the cell culture media is supplemented with a further cell culture media between days 15 and 17 of the rapid second expansion (including expansions referred to as REP). In some embodiments, the cell culture media is supplemented with a further cell culture media on day 16 of the rapid second expansion (including expansions referred to as REP). In some embodiments, the cell culture media is supplemented with a further cell culture media between days 9, 10, 11, 12, 13, 14, 15, 16, or 17 of the rapid second expansion (including expansions referred to as REP). In some embodiments, further cell culture media is comprises IL-2, OKT-3, and GlutaMAX™. In some embodiments, the further cell culture media is referred to as CM4, as described herein and in the Examples.

In some embodiments, the rapid second expansion (including expansions referred to as REP) is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

Optionally, a cell viability assay can be performed after the rapid second expansion (including expansions referred to as the REP expansion), using standard assays known in the art. For example, a trypan blue exclusion assay can be done on a sample of the bulk TILs, which selectively labels dead cells and allows a viability assessment. In some embodiments, TIL samples can be counted and viability determined using a Cellometer K2 automated cell counter (Nexcelom Bioscience, Lawrence, Mass.). In some embodiments, viability is determined according to the standard Cellometer K2 Image Cytometer Automatic Cell Counter protocol.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained in the second expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

In some embodiments, the rapid second expansion culture medium (e.g., sometimes referred to as CM2 or the second cell culture medium), comprises IL-2, OKT-3, as well as the antigen-presenting feeder cells (APCs), as discussed in more detail below. In some embodiments, the rapid second expansion culture medium (e.g., sometimes referred to as CM2 or the second cell culture medium), comprises 6000 IU/mL IL-2, 30 ug/flask OKT-3, as well as 7.5×10⁸ antigen-presenting feeder cells (APCs), as discussed in more detail below. In some embodiments, the rapid second expansion culture medium (e.g., sometimes referred to as CM2 or the second cell culture medium), comprises IL-2, OKT-3, as well as the antigen-presenting feeder cells (APCs), as discussed in more detail below. In some embodiments, the rapid second expansion culture medium (e.g., sometimes referred to as CM2 or the second cell culture medium), comprises 6000 IU/mL IL-2, 30 ug/flask OKT-3, as well as 5×10⁸ antigen-presenting feeder cells (APCs), as discussed in more detail below. In some embodiments, the rapid second expansion culture medium (e.g., sometimes referred to as CM2 or the second cell culture medium) comprises 2-mercaptoethanol (also referred to as beta-mercaptoethanol). In some embodiments, the rapid second expansion culture medium (e.g., sometimes referred to as CM2 or the second cell culture medium) comprises 55μ 2-mercaptoethanol.

In some embodiments, the rapid second expansion, for example, Step D according to FIG. 1 (in particular, e.g., FIG. 1B and/or FIG. 1C and/or FIG. 1D and/or FIG. 1E and/or FIG. 1F and/or FIG. 1G and/or FIG. 1H), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-100 or a G-REX-500. In some embodiments, the bioreactor employed is a G-REX-100. In some embodiments, the bioreactor employed is a G-REX-500.

1. Feeder Cells and Antigen Presenting Cells

In some embodiments, the rapid second expansion procedures described herein (for example including expansion such as those described in Step D from FIG. 1 (in particular, e.g., FIG. 1B and/or FIG. 1C and/or FIG. 1D and/or FIG. 1E and/or FIG. 1F and/or FIG. 1G and/or FIG. 1H), as well as those referred to as REP) require an excess of feeder cells during REP TIL expansion and/or during the rapid second expansion and/or culture supernatant from a culture of feeder cells (for example APCs) comprising OKT-3. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogenic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and acceptable for use in the TIL expansion procedures described herein if the total number of viable cells on day 7 or 14 is less than the initial viable cell number put into culture on day 0 of the REP and/or day 0 of the second expansion (i.e., the start day of the second expansion).

In some embodiments, PBMCs are considered replication incompetent and acceptable for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/ml OKT3 antibody and 3000 IU/ml IL-2. In some embodiments, the PBMCs are cultured in the presence of 60 ng/ml OKT3 antibody and 6000 IU/ml IL-2. In some embodiments, the PBMCs are cultured in the presence of 60 ng/ml OKT3 antibody and 3000 IU/ml IL-2. In some embodiments, the PBMCs are cultured in the presence of 30 ng/ml OKT3 antibody and 6000 IU/ml IL-2.

In some embodiments, PBMCs are considered replication incompetent and acceptable for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30-60 ng/ml OKT3 antibody and 1000-6000 IU/ml IL-2. In some embodiments, the PBMCs are cultured in the presence of 30-60 ng/ml OKT3 antibody and 2000-5000 IU/ml IL-2. In some embodiments, the PBMCs are cultured in the presence of 30-60 ng/ml OKT3 antibody and 2000-4000 IU/ml IL-2. In some embodiments, the PBMCs are cultured in the presence of 30-60 ng/ml OKT3 antibody and 2500-3500 IU/ml IL-2. In some embodiments, the PBMCs are cultured in the presence of 30-60 ng/ml OKT3 antibody and 6000 IU/ml IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 10, about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the second expansion procedures described herein require a ratio of about 5×10⁸ feeder cells to about 100×10⁶ TILs. In some embodiments, the second expansion procedures described herein require a ratio of about 7.5×10⁸ feeder cells to about 100×10⁶ TILs. In other embodiments, the second expansion procedures described herein require a ratio of about 5×10⁸ feeder cells to about 50×10⁶ TILs. In other embodiments, the second expansion procedures described herein require a ratio of about 7.5×10⁸ feeder cells to about 50×10⁶ TILs. In yet another embodiment, the second expansion procedures described herein require about 5×10⁸ feeder cells to about 25×10⁶ TILs. In yet another embodiment, the second expansion procedures described herein require about 7.5×10⁸ feeder cells to about 25×10⁶ TILs. In yet another embodiment, the rapid second expansion requires twice the number of feeder cells as the rapid second expansion. In yet another embodiment, when the priming first expansion described herein requires about 2.5×10⁸ feeder cells, the rapid second expansion requires about 5×10⁸ feeder cells. In yet another embodiment, when the priming first expansion described herein requires about 2.5×10⁸ feeder cells, the rapid second expansion requires about 7.5×10⁸ feeder cells. In yet another embodiment, the rapid second expansion requires two times (2.0×), 2.5×, 3.0×, 3.5× or 4.0× the number of feeder cells as the priming first expansion.

In some embodiments, the second expansion procedures described herein require a ratio of about 5×10⁸ feeder cells to about 100×10⁶ TILs. In some embodiments, the second expansion procedures described herein require a ratio of about 7.5×10⁸ feeder cells to about 100×10⁶ TILs. In other embodiments, the second expansion procedures described herein require a ratio of about 5×10⁸ feeder cells to about 50×10⁶ TILs. In other embodiments, the second expansion procedures described herein require a ratio of about 7.5×10⁸ feeder cells to about 50×10⁶ TILs. In yet another embodiment, the second expansion procedures described herein require about 5×10⁸ feeder cells to about 25×10⁶ TILs. In yet another embodiment, the second expansion procedures described herein require about 7.5×10⁸ feeder cells to about 25×10⁶ TILs. In yet another embodiment, the rapid second expansion requires the same number of feeder cells as the rapid second expansion. In yet another embodiment, when the priming first expansion described herein requires about 2.5×10⁸ feeder cells, the rapid second expansion requires about 2.5×10⁸ feeder cells. In yet another embodiment, when the priming first expansion described herein requires about 5×10⁸ feeder cells, the rapid second expansion requires about 5×10⁸ feeder cells. In yet another embodiment, when the priming first expansion described herein requires about 7.5×10⁸ feeder cells, the rapid second expansion requires about 7.5×10⁸ feeder cells. In yet another embodiment, the rapid second expansion requires two times (2.0×), 2.5×, 3.0×, 3.5× or 4.0× the number of feeder cells as the priming first expansion.

In some embodiments, the second expansion procedures described herein require a ratio of about 5×10⁸ feeder cells to about 100×10⁶ TILs. In some embodiments, the second expansion procedures described herein require a ratio of about 7.5×10⁸ feeder cells to about 100×10⁶ TILs. In other embodiments, the second expansion procedures described herein require a ratio of about 5×10⁸ feeder cells to about 50×10⁶ TILs. In other embodiments, the second expansion procedures described herein require a ratio of about 7.5×10⁸ feeder cells to about 50×10⁶ TILs. In yet another embodiment, the second expansion procedures described herein require about 5×10⁸ feeder cells to about 25×10⁶ TILs. In yet another embodiment, the second expansion procedures described herein require about 7.5×10⁸ feeder cells to about 25×10⁶ TILs. In yet another embodiment, the rapid second expansion requires the same number of feeder cells as the rapid second expansion. In yet another embodiment, when the priming first expansion described herein requires about 2.5×10⁸ feeder cells, the rapid second expansion requires about 2.5×10⁸ feeder cells. In yet another embodiment, when the priming first expansion described herein requires about 5×10⁸ feeder cells, the rapid second expansion requires about 5×10⁸ feeder cells. In yet another embodiment, when the priming first expansion described herein requires about 7.5×10⁸ feeder cells, the rapid second expansion requires about 7.5×10⁸ feeder cells.

In some embodiments, the rapid second expansion procedures described herein require an excess of feeder cells during the rapid second expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, artificial antigen-presenting (aAPC) cells are used in place of PBMCs. In some embodiments, the PBMCs are added to the rapid second expansion at twice the concentration of PBMCs that were added to the priming first expansion.

In general, the allogenic PBMCs are inactivated, either via irradiation or heat treatment, and used in the TIL expansion procedures described herein, including the exemplary procedures described in the figures and examples.

In some embodiments, artificial antigen presenting cells are used in the rapid second expansion as a replacement for, or in combination with, PBMCs.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is melanoma. In some embodiments, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, therapeutically effective dosage is about 2.3×10¹⁰ to about 13.7×10¹⁰. In some embodiments, therapeutically effective dosage is about 7.8×10¹⁰ TILs, particularly of the cancer is melanoma. In some embodiments, therapeutically effective dosage is about 1.2×10¹⁰ to about 4.3×10¹⁰ of TILs. In some embodiments, therapeutically effective dosage is about 3×10¹⁰ to about 12×10¹⁰ TILs. In some embodiments, therapeutically effective dosage is about 4×10¹⁰ to about 10×10¹⁰ TILs. In some embodiments, therapeutically effective dosage is about 5×10¹⁰ to about 8×10¹⁰ TILs. In some embodiments, therapeutically effective dosage is about 6×10¹⁰ to about 8×10¹⁰ TILs. In some embodiments, therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

2. Cytokines

The rapid second expansion methods described herein generally use culture media with high doses of a cytokine, in particular IL-2, as is known in the art.

Alternatively, using combinations of cytokines for the rapid second expansion of TILs is additionally possible, with combinations of two or more of IL-2, IL-15 and IL-21 as is generally outlined in International Publication No. WO 2015/189356 and WO 2015/189357, hereby expressly incorporated by reference in their entirety. Thus, possible combinations include IL-2 and IL-15, IL-2 and IL-21, IL-15 and IL-21 and IL-2, IL-15 and IL-21, with the latter finding particular use in many embodiments. The use of combinations of cytokines specifically favors the generation of lymphocytes, and in particular T-cells as described therein.

E. STEP E: Harvest TILs

After the rapid second expansion step, cells can be harvested. In some embodiments the TILs are harvested after one, two, three, four or more expansion steps, for example as provided in FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments the TILs are harvested after two expansion steps, for example as provided in FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments the TILs are harvested after two expansion steps, one priming first expansion and one rapid second expansion, for example as provided in FIG. 1 (in particular, e.g., FIG. 1B).

TILs can be harvested in any appropriate and sterile manner, including for example by centrifugation. Methods for TIL harvesting are well known in the art and any such known methods can be employed with the present process. In some embodiments, TILs are harvested using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell based harvester can be employed with the present methods. In some embodiments, the cell harvester and/or cell processing system is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the rapid second expansion, for example, Step D according to FIG. 1 (in particular, e.g., FIG. 1B and/or FIG. 1C and/or FIG. 1D and/or FIG. 1E and/or FIG. 1F and/or FIG. 1G and/or FIG. 1H), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-100 or a G-REX-500. In some embodiments, the bioreactor employed is a G-REX-100. In some embodiments, the bioreactor employed is a G-REX-500.

In some embodiments, Step E according to FIG. 1 (in particular, e.g., FIG. 1B and/or FIG. 1C and/or FIG. 1D and/or FIG. 1E and/or FIG. 1F and/or FIG. 1G and/or FIG. 1H), is performed according to the processes described herein. In some embodiments, the closed system is accessed via syringes under sterile conditions in order to maintain the sterility and closed nature of the system. In some embodiments, a closed system as described herein is employed.

In some embodiments, TILs are harvested according to the methods described herein. In some embodiments, TILs between days 14 and 16 are harvested using the methods as described herein. In some embodiments, TILs are harvested at 14 days using the methods as described herein. In some embodiments, TILs are harvested at 15 days using the methods as described herein. In some embodiments, TILs are harvested at 16 days using the methods as described herein.

F. STEP F: Final Formulation/Transfer to Infusion Bag

After Steps A through E as provided in an exemplary order in FIG. 1 (in particular, e.g., FIG. 1B and/or FIG. 1C and/or FIG. 1D and/or FIG. 1E and/or FIG. 1F and/or FIG. 1G and/or FIG. 1H) and as outlined in detailed above and herein are complete, cells are transferred to a container for use in administration to a patient. In some embodiments, once a therapeutically sufficient number of TILs are obtained using the expansion methods described above, they are transferred to a container for use in administration to a patient.

In some embodiments, TILs expanded using the methods of the present disclosure are administered to a patient as a pharmaceutical composition. In some embodiments, the pharmaceutical composition is a suspension of TILs in a sterile buffer. TILs expanded as disclosed herein may be administered by any suitable route as known in the art. In some embodiments, the TILs are administered as a single intra-arterial or intravenous infusion, which preferably lasts approximately 30 to 60 minutes. Other suitable routes of administration include intraperitoneal, intrathecal, and intralymphatic.

G. PBMC Feeder Cell Ratios

In some embodiments, the culture media used in expansion methods described herein (see for example, FIG. 1 (in particular, e.g., FIG. 1B and/or FIG. 1C and/or FIG. 1D and/or FIG. 1E and/or FIG. 1F and/or FIG. 1G and/or FIG. 1H) include an anti-CD3 antibody e.g. OKT-3. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J. Immunol. 1985, 135, 1719, hereby incorporated by reference in its entirety.

In some embodiments, the number of PBMC feeder layers is calculated as follows:
A. Volume of a T-cell (10 μm diameter): V=(4/3) πr³=523.6 μm³
B. Columne of G-Rex 100 (M) with a 40 μm (4 cells) height: V=(4/3) πr³=4×10¹² μm³
C. Number cell required to fill column B: 4×10¹² μm³/523.6 μm³=7.6×10⁸ μm³*0.64=4.86×10⁸
D. Number cells that can be optimally activated in 4D space: 4.86×10⁸/24=20.25×10⁶ E. Number of feeders and TIL extrapolated to G-Rex 500: TIL: 100×10⁶ and Feeder: 2.5×10⁹

In this calculation, an approximation of the number of mononuclear cells required to provide an icosahedral geometry for activation of TIL in a cylinder with a 100 cm² base is used. The calculation derives the experimental result of ˜5×10⁸ for threshold activation of T-cells which closely mirrors NCI experimental data.⁽¹⁾ (C) The multiplier (0.64) is the random packing density for equivalent spheres as calculated by Jaeger and Nagel in 1992⁽²⁾. (D) The divisor 24 is the number of equivalent spheres that could contact a similar object in 4 dimensional space “the Newton number.”⁽³⁾.

⁽¹⁾ Jin, Jianjian, et. al., Simplified Method of the Growth of Human Tumor Infiltrating Lymphocytes (TIL) in Gas-Permeable Flasks to Numbers Needed for Patient Treatment. J Immunother. 2012 April; 35(3): 283-292.

⁽²⁾Jaeger H M, Nagel S R. Physics of the granular state. Science. 1992 Mar. 20; 255(5051): 1523-31.

⁽³⁾O. R. Musin (2003). “The problem of the twenty-five spheres”. Russ. Math. Surv. 58 (4): 794-795.

In some embodiments, the number of antigen-presenting feeder cells exogenously supplied during the priming first expansion is approximately one-half the number of antigen-presenting feeder cells exogenously supplied during the rapid second expansion. In certain embodiments, the method comprises performing the priming first expansion in a cell culture medium which comprises approximately 50% fewer antigen presenting cells as compared to the cell culture medium of the rapid second expansion.

In other embodiments, the number of antigen-presenting feeder cells (APCs) exogenously supplied during the rapid second expansion is greater than the number of APCs exogenously supplied during the priming first expansion.

In other embodiments, the ratio of the number of APCs exogenously supplied during the rapid second expansion to the number of APCs exogenously supplied during the priming first expansion is selected from a range of from at or about 1.1:1 to at or about 20:1.

In other embodiments, the ratio of the number of APCs exogenously supplied during the rapid second expansion to the number of APCs exogenously supplied during the priming first expansion is selected from a range of from at or about 1.1:1 to at or about 10:1.

In other embodiments, the ratio of the number of APCs exogenously supplied during the rapid second expansion to the number of APCs exogenously supplied during the priming first expansion is selected from a range of from at or about 1.1:1 to at or about 9:1.

In other embodiments, the ratio of the number of APCs exogenously supplied during the rapid second expansion to the number of APCs exogenously supplied during the priming first expansion is selected from a range of from at or about 1.1:1 to at or about 8:1.

In other embodiments, the ratio of the number of APCs exogenously supplied during the rapid second expansion to the number of APCs exogenously supplied during the priming first expansion is selected from a range of from at or about 1.1:1 to at or about 7:1.

In other embodiments, the ratio of the number of APCs exogenously supplied during the rapid second expansion to the number of APCs exogenously supplied during the priming first expansion is selected from a range of from at or about 1.1:1 to at or about 6:1.

In other embodiments, the ratio of the number of APCs exogenously supplied during the rapid second expansion to the number of APCs exogenously supplied during the priming first expansion is selected from a range of from at or about 1.1:1 to at or about 5:1.

In other embodiments, the ratio of the number of APCs exogenously supplied during the rapid second expansion to the number of APCs exogenously supplied during the priming first expansion is selected from a range of from at or about 1.1:1 to at or about 4:1.

In other embodiments, the ratio of the number of APCs exogenously supplied during the rapid second expansion to the number of APCs exogenously supplied during the priming first expansion) is selected from a range of from at or about 1.1:1 to at or about 3:1.

In other embodiments, the ratio of the number of APCs exogenously supplied during the rapid second expansion to the number of APCs exogenously supplied during the priming first expansion is selected from a range of from at or about 1.1:1 to at or about 2.9:1.

In other embodiments, the ratio of the number of APCs exogenously supplied during the rapid second expansion to the number of APCs exogenously supplied during the priming first expansion is selected from a range of from at or about 1.1:1 to at or about 2.8:1.

In other embodiments, the ratio of the number of APCs exogenously supplied during the rapid second expansion to the number of APCs exogenously supplied during the priming first expansion is selected from a range of from at or about 1.1:1 to at or about 2.7:1.

In other embodiments, the ratio of the number of APCs exogenously supplied during the rapid second expansion to the number of APCs exogenously supplied during the priming first expansion is selected from a range of from at or about 1.1:1 to at or about 2.6:1.

In other embodiments, the ratio of the number of APCs exogenously supplied during the rapid second expansion to the number of APCs exogenously supplied during the priming first expansion is selected from a range of from at or about 1.1:1 to at or about 2.5:1.

In other embodiments, the ratio of the number of APCs exogenously supplied during the rapid second expansion to the number of APCs exogenously supplied during the priming first expansion is selected from a range of from at or about 1.1:1 to at or about 2.4:1.

In other embodiments, the ratio of the number of APCs exogenously supplied during the rapid second expansion to the number of APCs exogenously supplied during the priming first expansion is selected from a range of from at or about 1.1:1 to at or about 2.3:1.

In other embodiments, the ratio of the number of APCs exogenously supplied during the rapid second expansion to the number of APCs exogenously supplied during the priming first expansion is selected from a range of from at or about 1.1:1 to at or about 2.2:1.

In other embodiments, the ratio of the number of APCs exogenously supplied during the rapid second expansion to the number of APCs exogenously supplied during the priming first expansion is selected from a range of from at or about 1.1:1 to at or about 2.1:1.

In other embodiments, the ratio of the number of APCs exogenously supplied during the rapid second expansion to the number of APCs exogenously supplied during the priming first expansion is selected from a range of from at or about 1.1:1 to at or about 2:1.

In other embodiments, the ratio of the number of APCs exogenously supplied during the rapid second expansion to the number of APCs exogenously supplied during the priming first expansion is selected from a range of from at or about 2:1 to at or about 10:1.

In other embodiments, the ratio of the number of APCs exogenously supplied during the rapid second expansion to the number of APCs exogenously supplied during the priming first expansion is selected from a range of from at or about 2:1 to at or about 5:1.

In other embodiments, the ratio of the number of APCs exogenously supplied during the rapid second expansion to the number of APCs exogenously supplied during the priming first expansion is selected from a range of from at or about 2:1 to at or about 4:1.

In other embodiments, the ratio of the number of APCs exogenously supplied during the rapid second expansion to the number of APCs exogenously supplied during the priming first expansion is selected from a range of from at or about 2:1 to at or about 3:1.

In other embodiments, the ratio of the number of APCs exogenously supplied during the rapid second expansion to the number of APCs exogenously supplied during the priming first expansion is selected from a range of from at or about 2:1 to at or about 2.9:1.

In other embodiments, the ratio of the number of APCs exogenously supplied during the rapid second expansion to the number of APCs exogenously supplied during the priming first expansion is selected from a range of from at or about 2:1 to at or about 2.8:1.

In other embodiments, the ratio of the number of APCs exogenously supplied during the rapid second expansion to the number of APCs exogenously supplied during the priming first expansion is selected from a range of from at or about 2:1 to at or about 2.7:1.

In other embodiments, the ratio of the number of APCs exogenously supplied during the rapid second expansion to the number of APCs exogenously supplied during the priming first expansion is selected from a range of from at or about 2:1 to at or about 2.6:1.

In other embodiments, the ratio of the number of APCs exogenously supplied during the rapid second expansion to the number of APCs exogenously supplied during the priming first expansion is selected from a range of from at or about 2:1 to at or about 2.5:1.

In other embodiments, the ratio of the number of APCs exogenously supplied during the rapid second expansion to the number of APCs exogenously supplied during the priming first expansion is selected from a range of from at or about 2:1 to at or about 2.4:1.

In other embodiments, the ratio of the number of APCs exogenously supplied during the rapid second expansion to the number of APCs exogenously supplied during the priming first expansion is selected from a range of from at or about 2:1 to at or about 2.3:1.

In other embodiments, the ratio of the number of APCs exogenously supplied during the rapid second expansion to the number of APCs exogenously supplied during the priming first expansion is selected from a range of from at or about about 2:1 to at or about 2.2:1.

In other embodiments, the ratio of the number of APCs exogenously supplied during the rapid second expansion to the number of APCs exogenously supplied during the priming first expansion is selected from a range of from at or about 2:1 to at or about 2.1:1.

In other embodiments, the ratio of the number of APCs exogenously supplied during the rapid second expansion to the number of APCs exogenously supplied during the priming first expansion is at or about 2:1.

In other embodiments, the ratio of the number of APCs exogenously supplied during the rapid second expansion to the number of APCs exogenously supplied during the priming first expansion is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 12:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In other embodiments, the number of APCs exogenously supplied during the priming first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs, and the number of APCs exogenously supplied during the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs.

In other embodiments, the number of APCs exogenously supplied during the priming first expansion is selected from the range of at or about 1.5×10⁸ APCs to at or about 3×10⁸ APCs, and the number of APCs exogenously supplied during the rapid second expansion is selected from the range of at or about 4×10⁸ APCs to at or about 7.5×10⁸ APCs.

In other embodiments, the number of APCs exogenously supplied during the priming first expansion is selected from the range of at or about 2×10⁸ APCs to at or about 2.5×10⁸ APCs, and the number of APCs exogenously supplied during the rapid second expansion is selected from the range of at or about 4.5×10⁸ APCs to at or about 5.5×10⁸ APCs.

In other embodiments, the number of APCs exogenously supplied during the priming first expansion is at or about 2.5×10⁸ APCs, and the number of APCs exogenously supplied during the rapid second expansion is at or about 5×10⁸ APCs.

In some embodiments, the number of APCs (including, for example, PBMCs) added at day 0 of the priming first expansion is approximately one-half of the number of PBMCs added at day 7 of the priming first expansion (e.g., day 7 of the method). In certain embodiments, the method comprises adding antigen presenting cells at day 0 of the priming first expansion to the first population of TILs and adding antigen presenting cells at day 7 to the second population of TILs, wherein the number of antigen presenting cells added at day 0 is approximately 50% of the number of antigen presenting cells added at day 7 of the priming first expansion (e.g., day 7 of the method).

In other embodiments, the number of APCs (including, for example, PBMCs) exogenously supplied at day 7 of the rapid second expansion is greater than the number of PBMCs exogenously supplied at day 0 of the priming first expansion.

In other embodiments, the APCs exogenously supplied in the priming first expansion are seeded in the culture flask at a density selected from a range of at or about 1.0×10⁶ APCs/cm² to at or about 4.5×10⁶ APCs/cm².

In other embodiments, the APCs exogenously supplied in the priming first expansion are seeded in the culture flask at a density selected from a range of at or about 1.5×10⁶ APCs/cm² to at or about 3.5×10⁶ APCs/cm².

In other embodiments, the APCs exogenously supplied in the priming first expansion are seeded in the culture flask at a density selected from a range of at or about 2×10⁶ APCs/cm² to at or about 3×10⁶ APCs/cm².

In other embodiments, the APCs exogenously supplied in the priming first expansion are seeded in the culture flask at a density of at or about 2×10⁶ APCs/cm².

In other embodiments, the APCs exogenously supplied in the priming first expansion are seeded in the culture flask at a density of at or about 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶ or 4.5×10⁶ APCs/cm².

In other embodiments, the APCs exogenously supplied in the rapid second expansion are seeded in the culture flask at a density selected from a range of at or about 2.5×10⁶ APCs/cm² to at or about 7.5×10⁶ APCs/cm².

In other embodiments, the APCs exogenously supplied in the rapid second expansion are seeded in the culture flask at a density selected from a range of at or about 3.5×10⁶ APCs/cm² to about 6.0×10⁶ APCs/cm².

In other embodiments, the APCs exogenously supplied in the rapid second expansion are seeded in the culture flask at a density selected from a range of at or about 4.0×10⁶ APCs/cm² to about 5.5×10⁶ APCs/cm².

In other embodiments, the APCs exogenously supplied in the rapid second expansion are seeded in the culture flask at a density selected from a range of at or about 4.0×10⁶ APCs/cm².

In other embodiments, the APCs exogenously supplied in the rapid second expansion are seeded in the culture flask at a density of at or about 2.5×10⁶ APCs/cm², 2.6×10⁶ APCs/cm², 2.7×10⁶ APCs/cm², 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶, 7.1×10⁶, 7.2×10⁶, 7.3×10⁶, 7.4×10⁶ or 7.5×10⁶ APCs/cm².

In other embodiments, the APCs exogenously supplied in the priming first expansion are seeded in the culture flask at a density of at or about 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×106, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶ or 4.5×10⁶ APCs/cm² and the the APCs exogenously supplied in the rapid second expansion are seeded in the culture flask at a density of at or about 2.5×10⁶ APCs/cm², 2.6×10⁶ APCs/cm², 2.7×10⁶ APCs/cm², 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶, 7.1×10⁶, 7.2×10⁶, 7.3×10⁶, 7.4×10⁶ or 7.5×10⁶ APCs/cm².

In other embodiments, the APCs exogenously supplied in the priming first expansion are seeded in the culture flask at a density selected from a range of at or about 1.0×10⁶ APCs/cm² to at or about 4.5×10⁶ APCs/cm²; and the APCs exogenously supplied in the rapid second expansion are seeded in the culture flask at a density selected from a range of at or about 2.5×10⁶ APCs/cm² to at or about 7.5×10⁶ APCs/cm².

In other embodiments, the APCs exogenously supplied in the priming first expansion are seeded in the culture flask at a density selected from a range of at or about 1.5×10⁶ APCs/cm² to at or about 3.5×10⁶ APCs/cm²; and the APCs exogenously supplied in the rapid second expansion are seeded in the culture flask at a density selected from a range of at or about 3.5×10⁶ APCs/cm² to at or about 6×10⁶ APCs/cm².

In other embodiments, the APCs exogenously supplied in the priming first expansion are seeded in the culture flask at a density selected from a range of at or about 2×10⁶ APCs/cm² to at or about 3×10⁶ APCs/cm², and the APCs exogenously supplied in the rapid second expansion are seeded in the culture flask at a density selected from a range of at or about 4×10⁶ APCs/cm² to at or about 5.5×10⁶ APCs/cm².

In other embodiments, the APCs exogenously supplied in the priming first expansion are seeded in the culture flask at a density at or about 2×10⁶ APCs/cm² and the APCs exogenously supplied in the rapid second expansion are seeded in the culture flask at a density of at or about 4×10⁶ APCs/cm².

In other embodiments, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 20:1.

In other embodiments, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 10:1.

In other embodiments, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 9:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 8:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 7:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 6:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.9:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.8:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.7:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.6:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.1:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 10:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.9:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.8:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.7:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.6:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.1:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is at or about 2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In other embodiments, the number of APCs (including, for example, PBMCs) exogenously supplied at day 0 of the priming first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸ 3.4×10⁸ or 3.5×10⁸ APCs (including, for example, PBMCs), and the number of APCs (including, for example, PBMCs) exogenously supplied at day 7 of the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs (including, for example, PBMCs).

In other embodiments, the number of APCs (including, for example, PBMCs) exogenously supplied at day 0 of the priming first expansion is selected from the range of at or about 1×10⁸ APCs (including, for example, PBMCs) to at or about 3.5×10⁸ APCs (including, for example, PBMCs), and the number of APCs (including, for example, PBMCs) exogenously supplied at day 7 of the rapid second expansion is selected from the range of at or about 3.5×10⁸ APCs (including, for example, PBMCs) to at or about 1×10⁹ APCs (including, for example, PBMCs).

In other embodiments, the number of APCs (including, for example, PBMCs) exogenously supplied at day 0 of the priming first expansion is selected from the range of at or about 1.5×10⁸ APCs to at or about 3×10⁸ APCs (including, for example, PBMCs), and the number of APCs (including, for example, PBMCs) exogenously supplied at day 7 of the rapid second expansion is selected from the range of at or about 4×10⁸ APCs (including, for example, PBMCs) to at or about 7.5×10⁸ APCs (including, for example, PBMCs).

In other embodiments, the number of APCs (including, for example, PBMCs) exogenously supplied at day 0 of the priming first expansion is selected from the range of at or about 1×10⁸ APCs (including, for example, PBMCs) to at or about 3.5×10⁸ APCs (including, for example, PBMCs), and the number of APCs (including, for example, PBMCs) exogenously supplied at day 7 of the rapid second expansion is selected from the range of at or about 3.5×10⁸ APCs (including, for example, PBMCs) to at or about 1×10⁹ APCs (including, for example, PBMCs).

In other embodiments, the number of APCs (including, for example, PBMCs) exogenously supplied at day 0 of the priming first expansion is selected from the range of at or about 1.5×10⁸ APCs to at or about 3×10⁸ APCs (including, for example, PBMCs), and the number of APCs (including, for example, PBMCs) exogenously supplied at day 7 of the rapid second expansion is selected from the range of at or about 4×10⁸ APCs (including, for example, PBMCs) to at or about 7.5×10⁸ APCs (including, for example, PBMCs).

In other embodiments, the number of APCs (including, for example, PBMCs) exogenously supplied at day 0 of the priming first expansion is selected from the range of at or about 2×10⁸ APCs (including, for example, PBMCs) to at or about 2.5×10⁸ APCs (including, for example, PBMCs), and the number of APCs (including, for example, PBMCs) exogenously supplied at day 7 of the rapid second expansion is selected from the range of at or about 4.5×10⁸ APCs (including, for example, PBMCs) to at or about 5.5×10⁸ APCs (including, for example, PBMCs).

In other embodiments, the number of APCs (including, for example, PBMCs) exogenously supplied at day 0 of the priming first expansion is at or about 2.5×10⁸ APCs (including, for example, PBMCs) and the number of APCs (including, for example, PBMCs) exogenously supplied at day 7 of the rapid second expansion is at or about 5×10⁸ APCs (including, for example, PBMCs).

In some embodiments, the number of layers of APCs (including, for example, PBMCs) added at day 0 of the priming first expansion is approximately one-half of the number of layers of APCs (including, for example, PBMCs) added at day 7 of the rapid second expansion. In certain embodiments, the method comprises adding antigen presenting cell layers at day 0 of the priming first expansion to the first population of TILs and adding antigen presenting cell layers at day 7 to the second population of TILs, wherein the number of antigen presenting cell layer added at day 0 is approximately 50% of the number of antigen presenting cell layers added at day 7.

In other embodiments, the number of layers of APCs (including, for example, PBMCs) exogenously supplied at day 7 of the rapid second expansion is greater than the number of layers of APCs (including, for example, PBMCs) exogenously supplied at day 0 of the priming first expansion.

In other embodiments, day 0 of the priming first expansion occurs in the presence of layered APCs (including, for example, PBMCs) with an average thickness of at or about 2 cell layers and day 7 of the rapid second expansion occurs in the presence of layered APCs (including, for example, PBMCs) with an average thickness of at or about 4 cell layers.

In other embodiments, day 0 of the priming first expansion occurs in the presence of layered APCs (including, for example, PBMCs) with an average thickness of at or about one cell layer and day 7 of the rapid second expansion occurs in the presence of layered APCs (including, for example, PBMCs) with an average thickness of at or about 3 cell layers.

In other embodiments, day 0 of the priming first expansion occurs in the presence of layered APCs (including, for example, PBMCs) with an average thickness of at or about 1.5 cell layers to at or about 2.5 cell layers and day 7 of the rapid second expansion occurs in the presence of layered APCs (including, for example, PBMCs) with an average thickness of at or about 3 cell layers.

In other embodiments, day 0 of the priming first expansion occurs in the presence of layered APCs (including, for example, PBMCs) with an average thickness of at or about one cell layer and day 7 of the rapid second expansion occurs in the presence of layered APCs (including, for example, PBMCs) with an average thickness of at or about 2 cell layers.

In other embodiments, day 0 of the priming first expansion occurs in the presence of layered APCs (including, for example, PBMCs) with an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers and day 7 of the rapid second expansion occurs in the presence of layered APCs (including, for example, PBMCs) with an average thickness of at or about 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, day 0 of the priming first expansion occurs in the presence of layered APCs (including, for example, PBMCs) with an average thickness of at or about 1 cell layer to at or about 2 cell layers and day 7 of the rapid second expansion occurs in the presence of layered APCs (including, for example, PBMCs) with an average thickness of at or about 3 cell layers to at or about 10 cell layers.

In other embodiments, day 0 of the priming first expansion occurs in the presence of layered APCs (including, for example, PBMCs) with an average thickness of at or about 2 cell layers to at or about 3 cell layers and day 7 of the rapid second expansion occurs in the presence of layered APCs (including, for example, PBMCs) with an average thickness of at or about 4 cell layers to at or about 8 cell layers.

In other embodiments, day 0 of the priming first expansion occurs in the presence of layered APCs (including, for example, PBMCs) with an average thickness of at or about 2 cell layers and day 7 of the rapid second expansion occurs in the presence of layered APCs (including, for example, PBMCs) with an average thickness of at or about 4 cell layers to at or about 8 cell layers.

In other embodiments, day 0 of the priming first expansion occurs in the presence of layered APCs (including, for example, PBMCs) with an average thickness of at or about 1, 2 or 3 cell layers and day 7 of the rapid second expansion occurs in the presence of layered APCs (including, for example, PBMCs) with an average thickness of at or about 3, 4, 5, 6, 7, 8, 9 or 10 cell layers.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:10.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:8.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:7.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:6.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:4.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:3.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:2.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.2 to at or about 1:8.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.3 to at or about 1:7.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.4 to at or about 1:6.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.5 to at or about 1:5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.6 to at or about 1:4.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.7 to at or about 1:3.5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.8 to at or about 1:3.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.9 to at or about 1:2.5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is at or about 1:2.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from at or about 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9 or 1:10.

In some embodiments, the number of APCs in the priming first expansion is selected from the range of about 1.0×10⁶ APCs/cm² to about 4.5×10⁶ APCs/cm², and the number of APCs in the rapid second expansion is selected from the range of about 2.5×10⁶ APCs/cm² to about 7.5×10⁶ APCs/cm².

In some embodiments, the number of APCs in the priming first expansion is selected from the range of about 1.5×10⁶ APCs/cm² to about 3.5×10⁶ APCs/cm², and the number of APCs in the rapid second expansion is selected from the range of about 3.5×10⁶ APCs/cm² to about 6.0×10⁶ APCs/cm².

In some embodiments, the number of APCs in the priming first expansion is selected from the range of about 2.0×10⁶ APCs/cm² to about 3.0×10⁶ APCs/cm², and the number of APCs in the rapid second expansion is selected from the range of about 4.0×10⁶ APCs/cm² to about 5.5×10⁶ APCs/cm².

H. Optional Cell Medium Components

1. Anti-CD3 Antibodies

In some embodiments, the culture media used in expansion methods described herein (see for example, FIG. 1 (in particular, e.g., FIG. 1B and/or FIG. 1C and/or FIG. 1D and/or FIG. 1E and/or FIG. 1F and/or FIG. 1G and/or FIG. 1H) include an anti-CD3 antibody. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J. Immunol. 1985, 135, 1719, hereby incorporated by reference in its entirety.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In particular embodiments, the OKT3 anti-CD3 antibody is used (commercially available from Ortho-McNeil, Raritan, NJ or Miltenyi Biotech, Auburn, Calif.).

2. 4-1BB (CD137) AGONISTS

In some embodiments, the cell culture medium of the priming first expansion and/or the rapid second expansion comprises a TNFRSF agonist. In some embodiments, the TNFRSF agonist is a 4-1BB (CD137) agonist. The 4-1BB agonist may be any 4-1BB binding molecule known in the art. The 4-1BB binding molecule may be a monoclonal antibody or fusion protein capable of binding to human or mammalian 4-1BB. The 4-1BB agonists or 4-1BB binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The 4-1BB agonist or 4-1BB binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to 4-1BB. In some embodiments, the 4-1BB agonist is an antigen binding protein that is a fully human antibody. In some embodiments, the 4-1BB agonist is an antigen binding protein that is a humanized antibody. In some embodiments, 4-1BB agonists for use in the presently disclosed methods and compositions include anti-4-1BB antibodies, human anti-4-1BB antibodies, mouse anti-4-1BB antibodies, mammalian anti-4-1BB antibodies, monoclonal anti-4-1BB antibodies, polyclonal anti-4-1BB antibodies, chimeric anti-4-1BB antibodies, anti-4-1BB adnectins, anti-4-1BB domain antibodies, single chain anti-4-1BB fragments, heavy chain anti-4-1BB fragments, light chain anti-4-1BB fragments, anti-4-1BB fusion proteins, and fragments, derivatives, conjugates, variants, or biosimilars thereof. Agonistic anti-4-1BB antibodies are known to induce strong immune responses. Lee, et al., PLOS One 2013, 8, e69677. In some embodiments, the 4-1BB agonist is an agonistic, anti-4-1BB humanized or fully human monoclonal antibody (i.e., an antibody derived from a single cell line). In some embodiments, the 4-1BB agonist is EU-101 (Eutilex Co. Ltd.), utomilumab, or urelumab, or a fragment, derivative, conjugate, variant, or biosimilar thereof. In some embodiments, the 4-1BB agonist is utomilumab or urelumab, or a fragment, derivative, conjugate, variant, or biosimilar thereof.

In some embodiments, the 4-1BB agonist or 4-1BB binding molecule may also be a fusion protein. In some embodiments, a multimeric 4-1BB agonist, such as a trimeric or hexameric 4-1BB agonist (with three or six ligand binding domains), may induce superior receptor (4-1BBL) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic 4-1BB antibodies and fusion proteins are known to induce strong immune responses. In some embodiments, the 4-1BB agonist is a monoclonal antibody or fusion protein that binds specifically to 4-1BB antigen in a manner sufficient to reduce toxicity. In some embodiments, the 4-1BB agonist is an agonistic 4-1BB monoclonal antibody or fusion protein that abrogates antibody-dependent cellular toxicity (ADCC), for example NK cell cytotoxicity. In some embodiments, the 4-1BB agonist is an agonistic 4-1BB monoclonal antibody or fusion protein that abrogates antibody-dependent cell phagocytosis (ADCP). In some embodiments, the 4-1BB agonist is an agonistic 4-1BB monoclonal antibody or fusion protein that abrogates complement-dependent cytotoxicity (CDC). In some embodiments, the 4-1BB agonist is an agonistic 4-1BB monoclonal antibody or fusion protein which abrogates Fc region functionality.

In some embodiments, the 4-1BB agonists are characterized by binding to human 4-1BB (SEQ ID NO:9) with high affinity and agonistic activity. In some embodiments, the 4-1BB agonist is a binding molecule that binds to human 4-1BB (SEQ ID NO:9). In some embodiments, the 4-1BB agonist is a binding molecule that binds to murine 4-1BB (SEQ ID NO:10). The amino acid sequences of 4-1BB antigen to which a 4-1BB agonist or binding molecule binds are summarized in Table 6.

TABLE 6
Amino acid sequences of 4-1BB antigens.

| Identifier | Sequence (One-Letter Amino Acid Symbols) | |
|---|---|---|
| SEQ ID NO: 9 | MGNSCYNIVA TLLLVLNFER TRSLQDPCSN CPAGTFCDNN RNQICSPCPP NSFSSAGGQR | 60 |
| human 4-1BB, | TCDICRQCKG VERTRKECSS TSNAECDCTP GFHCLGAGCS MCEQDCKQGQ ELTKKGCKDC | 120 |
| Tumor necrosis | CFGTFNDQKR GICRPWTNCS LDGKSVLVNG TKERDVVCGP SPADLSPGAS SVTPPAPARE | 180 |
| factor receptor | PGHSPQIISF FLALTSTALL FLLFFLTLRF SVVKRGRKKL LYIFKQPFMR PVQTTQEEDG | 240 |
| superfamily, | CSCRFPEEEE GGCEL | 255 |
| member 9 (Homo | | |
| sapiens) | | |
| SEQ ID NO: 10 | MGNNCYNVVV IVLLLVGCEK VGAVQNSCDN CQPGTFCRKY NPVCKSCPPS TFSSIGGQPN | 60 |
| murine 4-1BB, | CNICRVCAGY FREKKFCSST HNAECECIEG FHCLGPQCTR CEKDCRPGQE LTKQGCKTCS | 120 |
| Tumor necrosis | LGTFNDQNGT GVCRPWTNCS LDGRSVLKTG TTEKDVVCGP PVVSFSPSTT ISVTPEGGPG | 180 |
| factor receptor | GHSLQVLTLF LALTSALLLA LIFITLLFSV LKWIRKKFPH IFKQPFKKTT GAAQEEDACS | 240 |
| superfamily, | CRCPQEEEGG GGGYEL | 256 |
| member 9 (Mus | | |
| musculus) | | |

In some embodiments, the compositions, processes and methods described include a 4-1BB agonist that binds human or murine 4-1BB with a K_{D} of about 100 pM or lower, binds human or murine 4-1BB with a K_{D} of about 90 pM or lower, binds human or murine 4-1BB with a K_{D} of about 80 pM or lower, binds human or murine 4-1BB with a K_{D} of about 70 pM or lower, binds human or murine 4-1BB with a K_{D} of about 60 pM or lower, binds human or murine 4-1BB with a K_{D} of about 50 pM or lower, binds human or murine 4-1BB with a K_{D} of about 40 pM or lower, or binds human or murine 4-1BB with a K_{D} of about 30 pM or lower.

In some embodiments, the compositions, processes and methods described include a 4-1BB agonist that binds to human or murine 4-1BB with a k_{assoc} of about 7.5×10⁵ 1/M·s or faster, binds to human or murine 4-1BB with a k_{assoc} of about 7.5×10⁵ 1/M·s or faster, binds to human or murine 4-1BB with a k_{assoc} of about 8×10⁵ 1/M·s or faster, binds to human or murine 4-1BB with a k_{assoc} of about 8.5×10⁵ 1/M·s or faster, binds to human or murine 4-1BB with a k_{assoc} of about 9×10⁵ 1/M·s or faster, binds to human or murine 4-1BB with a k_{assoc} of about 9.5×10⁵ 1/M·s or faster, or binds to human or murine 4-1BB with a k_{assoc} of about 1×10⁶ 1/M·s or faster.

In some embodiments, the compositions, processes and methods described include a 4-1BB agonist that binds to human or murine 4-1BB with a k_{dissoc} of about 2×10⁻⁵ 1/s or slower, binds to human or murine 4-1BB with a k_{dissoc} of about 2.1×10⁻⁵ 1/s or slower, binds to human or murine 4-1BB with a k_{dissoc} of about 2.2×10⁻⁵ 1/s or slower, binds to human or murine 4-1BB with a k_{dissoc} of about 2.3×10⁻⁵ 1/s or slower, binds to human or murine 4-1BB with a k_{dissoc} of about 2.4×10⁻⁵ 1/s or slower, binds to human or murine 4-1BB with a k_{dissoc} of about 2.5×10⁻⁵ 1/s or slower, binds to human or murine 4-1BB with a k_{dissoc} of about 2.6×10⁻⁵ 1/s or slower or binds to human or murine 4-1BB with a k_{dissoc} of about 2.7×10⁻⁵ 1/s or slower, binds to human or murine 4-1BB with a k_{dissoc} of about 2.8×10⁻⁵ 1/s or slower, binds to human or murine 4-1BB with a k_{dissoc} of about 2.9×10⁻⁵ 1/s or slower, or binds to human or murine 4-1BB with a k_{dissoc} of about 3×10⁻⁵ 1/s or slower.

In some embodiments, the compositions, processes and methods described include a 4-1BB agonist that binds to human or murine 4-1BB with an IC₅₀ of about 10 nM or lower, binds to human or murine 4-1BB with an IC₅₀ of about 9 nM or lower, binds to human or murine 4-1BB with an IC₅₀ of about 8 nM or lower, binds to human or murine 4-1BB with an IC₅₀ of about 7 nM or lower, binds to human or murine 4-1BB with an IC₅₀ of about 6 nM or lower, binds to human or murine 4-1BB with an IC₅₀ of about 5 nM or lower, binds to human or murine 4-1BB with an IC₅₀ of about 4 nM or lower, binds to human or murine 4-1BB with an IC₅₀ of about 3 nM or lower, binds to human or murine 4-1BB with an IC₅₀ of about 2 nM or lower, or binds to human or murine 4-1BB with an IC₅₀ of about 1 nM or lower.

In some embodiments, the 4-1BB agonist is utomilumab, also known as PF-05082566 or MOR-7480, or a fragment, derivative, variant, or biosimilar thereof. Utomilumab is available from Pfizer, Inc. Utomilumab is an immunoglobulin G2-lambda, anti-[Homo sapiens TNFRSF9 (tumor necrosis factor receptor (TNFR) superfamily member 9, 4-1BB, T cell antigen ILA, CD137)], Homo sapiens (fully human) monoclonal antibody. The amino acid sequences of utomilumab are set forth in Table 7. Utomilumab comprises glycosylation sites at Asn59 and Asn292; heavy chain intrachain disulfide bridges at positions 22-96 (V_{H}-V_{L}), 143-199 (C_{H}1-C_{L}), 256-316 (C_{H}2) and 362-420 (C_{H}3); light chain intrachain disulfide bridges at positions 22′-87′ (V_{H}-V_{L}) and 136′-195′ (C_{H}1-C_{L}); interchain heavy chain-heavy chain disulfide bridges at IgG2A isoform positions 218-218, 219-219, 222-222, and 225-225, at IgG2A/B isoform positions 218-130, 219-219, 222-222, and 225-225, and at IgG2B isoform positions 219-130 (2), 222-222, and 225-225; and interchain heavy chain-light chain disulfide bridges at IgG2A isoform positions 130-213′ (2), IgG2A/B isoform positions 218-213′ and 130-213′, and at IgG2B isoform positions 218-213′ (2). The preparation and properties of utomilumab and its variants and fragments are described in U.S. Pat. Nos. 8,821,867; 8,337,850; and 9,468,678, and International Patent Application Publication No. WO 2012/032433 A1, the disclosures of each of which are incorporated by reference herein. Preclinical characteristics of utomilumab are described in Fisher, et al., Cancer Immunolog. & Immunother. 2012, 61, 1721-33. Current clinical trials of utomilumab in a variety of hematological and solid tumor indications include U.S. National Institutes of Health clinicaltrials.gov identifiers NCT02444793, NCT01307267, NCT02315066, and NCT02554812.

In some embodiments, a 4-1BB agonist comprises a heavy chain given by SEQ ID NO:11 and a light chain given by SEQ ID NO:12. In some embodiments, a 4-1BB agonist comprises heavy and light chains having the sequences shown in SEQ ID NO:11 and SEQ ID NO:12, respectively, or antigen binding fragments, Fab fragments, single-chain variable fragments (scFv), variants, or conjugates thereof. In some embodiments, a 4-1BB agonist comprises heavy and light chains that are each at least 99% identical to the sequences shown in SEQ ID NO:11 and SEQ ID NO:12, respectively. In some embodiments, a 4-1BB agonist comprises heavy and light chains that are each at least 98% identical to the sequences shown in SEQ ID NO:11 and SEQ ID NO:12, respectively. In some embodiments, a 4-1BB agonist comprises heavy and light chains that are each at least 97% identical to the sequences shown in SEQ ID NO:11 and SEQ ID NO:12, respectively. In some embodiments, a 4-1BB agonist comprises heavy and light chains that are each at least 96% identical to the sequences shown in SEQ ID NO:11 and SEQ ID NO:12, respectively. In some embodiments, a 4-1BB agonist comprises heavy and light chains that are each at least 95% identical to the sequences shown in SEQ ID NO:11 and SEQ ID NO:12, respectively.

In some embodiments, the 4-1BB agonist comprises the heavy and light chain CDRs or variable regions (VRs) of utomilumab. In some embodiments, the 4-1BB agonist heavy chain variable region (VH) comprises the sequence shown in SEQ ID NO:13, and the 4-1BB agonist light chain variable region (VL) comprises the sequence shown in SEQ ID NO:14, and conservative amino acid substitutions thereof. In some embodiments, a 4-1BB agonist comprises V_{H} and V_{L} regions that are each at least 99% identical to the sequences shown in SEQ ID NO:13 and SEQ ID NO:14, respectively. In some embodiments, a 4-1BB agonist comprises V_{H} and V_{L} regions that are each at least 98% identical to the sequences shown in SEQ ID NO:13 and SEQ ID NO:14, respectively. In some embodiments, a 4-1BB agonist comprises V_{H} and V_{L} regions that are each at least 97% identical to the sequences shown in SEQ ID NO:13 and SEQ ID NO:14, respectively. In some embodiments, a 4-1BB agonist comprises V_{H} and V_{L} regions that are each at least 96% identical to the sequences shown in SEQ ID NO:13 and SEQ ID NO:14, respectively. In some embodiments, a 4-1BB agonist comprises V_{H} and V_{L} regions that are each at least 95% identical to the sequences shown in SEQ ID NO:13 and SEQ ID NO:14, respectively. In some embodiments, a 4-1BB agonist comprises an scFv antibody comprising V_{H} and V_{L} regions that are each at least 99% identical to the sequences shown in SEQ ID NO:13 and SEQ ID NO:14.

In some embodiments, a 4-1BB agonist comprises heavy chain CDR1, CDR2 and CDR3 domains having the sequences set forth in SEQ ID NO:15, SEQ ID NO:16, and SEQ ID NO:17, respectively, and conservative amino acid substitutions thereof, and light chain CDR1, CDR2 and CDR3 domains having the sequences set forth in SEQ ID NO:18, SEQ ID NO:19, and SEQ ID NO:20, respectively, and conservative amino acid substitutions thereof.

In some embodiments, the 4-1BB agonist is a 4-1BB agonist biosimilar monoclonal antibody approved by drug regulatory authorities with reference to utomilumab. In some embodiments, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is utomilumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is utomilumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab.

TABLE 7
Amino acid sequences for 4-1BB agonist antibodies related to utomilumab.

| Identifier | Sequence (One-Letter Amino Acid Symbols) | |
|---|---|---|
| SEQ ID NO: 11 | EVQLVQSGAE VKKPGESLRI SCKGSGYSFS TYWISWVRQM PGKGLEWMGK IYPGDSYTNY | 60 |
| heavy chain for | SPSFQGQVTI SADKSISTAY LQWSSLKASD TAMYYCARGY GIFDYWGQGT LVTVSSASTK | 120 |
| utomilumab | GPSVFPLAPC SRSTSESTAA IGCLVKDYFP EPVTVSWNSG ALTSGVHTFP AVLQSSGLYS | 180 |
| | LSSVVTVPSS NFGTQTYTCN VDHKPSNTKV DKTVERKCCV ECPPCPAPPV AGPSVFLFPP | 240 |
| | KPKDTLMISR TPEVTCVVVD VSHEDPEVQF NWYVDGVEVI NAKTKPREEQ FNSTFRVVSV | 300 |
| | LTVVHQDWIN GKEYKCKVSN KGLPAPIEKT ISKTKGQPRE PQVYTLPPSR EEMTRNQVSL | 360 |
| | TCLVKGFYPS DIAVEWESNG CPENNYKTTP PMLDSDGSFF LYSKLTVDKS RWQQGNVFSC | 420 |
| | SVMHEALHNH YTQKSLSLSP G | 441 |
| SEQ ID NO: 12 | SYELTQPPSV SVSPGQTASI TCSGDNIGDQ YAHWYQQKPG QSPVLVIYQD KNRPSGIPER | 60 |
| light chain for | FSGSNSGNTA TLTISGTQAM DEADYYCATY TGFGSLAVFG GGTKLTVLGQ PKAAPSVTLF | 120 |
| utomilumab | PPSSEELQAN KATLVCLISD FYPGAVTVAW KADSSPVKAG VETTTPSKQS NNKYAASSYL | 180 |
| | SLTPEQWKSH RSYSCQVTHE GSTVEKTVAP TECS | 214 |
| SEQ ID NO: 13 | EVQLVQSGAE VKKPGESLRI SCKGSGYSFS TYWISWVRQM PGKGLEWMG KIYPGDSYTN | 60 |
| heavy chain | YSPSFQGQVT ISADKSISTA YLCWSSLKAS DTAMYYCARG YGIFDYWGQ GTLVTVSS | 118 |
| variable region | | |
| for utomilumab | | |
| SEQ ID NO: 14 | SYELTQPPSV SVSPGQTASI TCSGDNIGDQ YAHWYQQKPG QSPVLVIYQD KNRPSGIPER | 60 |
| light chain | FSGSNSGNTA TLTISGTQAM DEADYYCATY TGFGSLAVFG GGTKLTVL | 108 |
| variable region | | |
| for utomilumab | | |
| SEQ ID NO: 15 | STYWIS | 6 |
| heavy chain CDR1 | | |
| for utomilumab | | |
| SEQ ID NO: 16 | KIYPGDSYTN YSPSFQG | 17 |
| heavy chain CDR2 | | |
| for utomilumab | | |
| SEQ ID NO: 17 | RGYGIFDY | 8 |
| heavy chain CDR3 | | |
| for utomilumab | | |
| SEQ ID NO: 18 | SGDNIGDQYA H | 11 |
| light chain CDR1 | | |
| for utomilumab | | |
| SEQ ID NO: 19 | QDKNRPS | 7 |
| light chain CDR2 | | |
| for utomilumab | | |
| SEQ ID NO: 20 | ATYTGFGSLA V | 11 |
| light chain CDR3 | | |
| for utomilumab | | |

In some embodiments, the 4-1BB agonist is the monoclonal antibody urelumab, also known as BMS-663513 and 20H4.9.h4a, or a fragment, derivative, variant, or biosimilar thereof. Urelumab is available from Bristol-Myers Squibb, Inc., and Creative Biolabs, Inc. Urelumab is an immunoglobulin G4-kappa, anti-[Homo sapiens TNFRSF9 (tumor necrosis factor receptor superfamily member 9, 4-1BB, T cell antigen ILA; CD137)], Homo sapiens (fully human) monoclonal antibody. The amino acid sequences of urelumab are set forth in Table 8. Urelumab comprises N-glycosylation sites at positions 298 (and 298″); heavy chain intrachain disulfide bridges at positions 22-95 (V_{H}-V_{L}), 148-204 (C_{H}1-C_{L}), 262-322 (C_{H}2) and 368-426 (C_{H}3) (and at positions 22″-95″, 148″-204″, 262″-322″, and 368″-426″); light chain intrachain disulfide bridges at positions 23′-88′ (V_{H}-V_{L}) and 136′-196′ (C_{H}1-C_{L}) (and at positions 23′″−88′″ and 136′″−196′″); interchain heavy chain-heavy chain disulfide bridges at positions 227-227″ and 230-230″; and interchain heavy chain-light chain disulfide bridges at 135-216′ and 135″-216″. The preparation and properties of urelumab and its variants and fragments are described in U.S. Pat. Nos. 7,288,638 and 8,962,804, the disclosures of which are incorporated by reference herein. The preclinical and clinical characteristics of urelumab are described in Segal, et al., Clin. Cancer Res. 2016, available at http:/dx.doi.org/10.1158/1078-0432.CCR-16-1272. Current clinical trials of urelumab in a variety of hematological and solid tumor indications include U.S. National Institutes of Health clinicaltrials.gov identifiers NCT01775631, NCT02110082, NCT02253992, and NCT01471210.

In some embodiments, a 4-1BB agonist comprises a heavy chain given by SEQ ID NO:21 and a light chain given by SEQ ID NO:22. In some embodiments, a 4-1BB agonist comprises heavy and light chains having the sequences shown in SEQ ID NO:21 and SEQ ID NO:22, respectively, or antigen binding fragments, Fab fragments, single-chain variable fragments (scFv), variants, or conjugates thereof. In some embodiments, a 4-1BB agonist comprises heavy and light chains that are each at least 99% identical to the sequences shown in SEQ ID NO:21 and SEQ ID NO:22, respectively. In some embodiments, a 4-1BB agonist comprises heavy and light chains that are each at least 98% identical to the sequences shown in SEQ ID NO:21 and SEQ ID NO:22, respectively. In some embodiments, a 4-1BB agonist comprises heavy and light chains that are each at least 97% identical to the sequences shown in SEQ ID NO:21 and SEQ ID NO:22, respectively. In some embodiments, a 4-1BB agonist comprises heavy and light chains that are each at least 96% identical to the sequences shown in SEQ ID NO:21 and SEQ ID NO:22, respectively. In some embodiments, a 4-1BB agonist comprises heavy and light chains that are each at least 95% identical to the sequences shown in SEQ ID NO:21 and SEQ ID NO:22, respectively.

In some embodiments, the 4-1BB agonist comprises the heavy and light chain CDRs or variable regions (VRs) of urelumab. In some embodiments, the 4-1BB agonist heavy chain variable region (V_{H}) comprises the sequence shown in SEQ ID NO:23, and the 4-1BB agonist light chain variable region (V_{L}) comprises the sequence shown in SEQ ID NO:24, and conservative amino acid substitutions thereof. In some embodiments, a 4-1BB agonist comprises V_{H} and V_{L} regions that are each at least 99% identical to the sequences shown in SEQ ID NO:23 and SEQ ID NO:24, respectively. In some embodiments, a 4-1BB agonist comprises Vii and V_{L} regions that are each at least 98% identical to the sequences shown in SEQ ID NO:23 and SEQ ID NO:24, respectively. In some embodiments, a 4-1BB agonist comprises V_{H} and V_{L} regions that are each at least 97% identical to the sequences shown in SEQ ID NO:23 and SEQ ID NO:24, respectively. In some embodiments, a 4-1BB agonist comprises V_{H} and V_{L} regions that are each at least 96% identical to the sequences shown in SEQ ID NO:23 and SEQ ID NO:24, respectively. In some embodiments, a 4-1BB agonist comprises V_{H} and V_{L} regions that are each at least 95% identical to the sequences shown in SEQ ID NO:23 and SEQ ID NO:24, respectively. In some embodiments, a 4-1BB agonist comprises an scFv antibody comprising V_{H} and V_{L} regions that are each at least 99% identical to the sequences shown in SEQ ID NO:23 and SEQ ID NO:24.

In some embodiments, a 4-1BB agonist comprises heavy chain CDR1, CDR2 and CDR3 domains having the sequences set forth in SEQ ID NO:25, SEQ ID NO:26, and SEQ ID NO:27, respectively, and conservative amino acid substitutions thereof, and light chain CDR1, CDR2 and CDR3 domains having the sequences set forth in SEQ ID NO:28, SEQ ID NO:29, and SEQ ID NO:30, respectively, and conservative amino acid substitutions thereof.

In some embodiments, the 4-1BB agonist is a 4-1BB agonist biosimilar monoclonal antibody approved by drug regulatory authorities with reference to urelumab. In some embodiments, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is urelumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is urelumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab.

TABLE 8
Amino acid sequences for 4-1BB agonist antibodies related to urelumab.

| Identifier | Sequence (One-Letter Amino Acid Symbols) | |
|---|---|---|
| SEQ ID NO: 21 | QVQLQQWGAG LLKPSETLSL TCAVYGGSFS GYYWSWIRQS PEKGLEWIGE INHGGYVTYN | 60 |
| heavy chain for | PSLESRVTIS VDTSKNQFSL KLSSVTAADT AVYYCARDYG PGNYDWYFDL WGRGTLVTVS | 120 |
| urelumab | SASTKGPSVF PLAPCSRSTS ESTAALGCLV KDYFPEPVTV SWNSGALTSG VHTFPAVLQS | 180 |
| | SGLYSLSSVV TVPSSSLGTK TYTCNVDHKP SNTKVDKRVE SKYGPPCPPC PAPEFLGGPS | 240 |
| | VFLFPPKPKD TLMISRTPEV TCVVVDVSQE DPEVQFNWYV DGVEVHNAKT KPREEQFNST | 300 |
| | YRVVSVLTVL HQDWLNGKEY KCKVSNKGLP SSIEKTISKA KGQPREPQVY TLPPSQEEMT | 360 |
| | KNQVSLTCLV KGFYPSDIAV EWESNGQPEN NYKTTPPVLD SDGSFFLYSR LTVDKSRWQE | 420 |
| | GNVFSCSVMH EALHNHYTQK SLSLSLGK | 448 |
| SEQ ID NO: 22 | EIVLTQSPAT LSLSPGERAT ISCRASQSVS SYLAWYQQKP GQAPRLLIYD ASNRATGIPA | |
| light chain for | RESGSGSGTD FTLTISSLEP EDFAVYYCQQ RSNWPPALTF CGGTKVEIKR TVAAPSVFIF | 120 |
| urelumab | PPSDEQLKSG TASVVCLLNN FYPREAKVQW KVDNALQSGN SQESVTEQDS KDSTYSLSST | 180 |
| | LTLSKADYEK HKVYACEVTH CGLSSPVTKS FNRGEC | 216 |
| SEQ ID NO: 23 | MKHLWFFLLL VAAPRWVLSQ VQLQQWGAGL LKPSETLSLT CAVYGGSFSG YYWSWIRQSP | 60 |
| variable heavy | EKGLEWIGEI NHGGYVTYNP SLESRVTISV DTSKNQFSLK LSSVTAADTA VYYCARDYGP | 120 |
| chain for | | |
| urelumab | | |
| SEQ ID NO: 24 | MEAPAQLLFL LLLWLPDTTG FIVLTQSPAT LSLSPGERAT LSCRASQSVS SYLAWYQQKP | 60 |
| variable light | GQAPRLLIYD ASNRATGIPA RESGSGSGTD FTLTISSLEP EDFAVYYCQQ | 110 |
| chain for | | |
| urelumab | | |
| SEQ ID NO: 25 | GYYWS | 5 |
| heavy chain CDR1 | | |
| for urelumab | | |
| SEQ ID NO: 26 | EINHGGYVTY NPSLES | 16 |
| heavy chain CDR2 | | |
| for urelumab | | |
| SEQ ID NO:27 | DYGPGNYDWY FDL | 13 |
| heavy chain CDR3 | | |
| for urelumab | | |
| SEQ ID NO: 28 | RASQSVSSYL A | 11 |
| light chain CDR1 | | |
| for urelumab | | |
| SEQ ID NO: 29 | DASNRAT | 7 |
| light chain CDR2 | | |
| for urelumab | | |
| SEQ ID NO: 30 | QQRSDWPPAL T | 11 |
| light chain CDR3 | | |
| for urelumab | | |

In some embodiments, the 4-1BB agonist is selected from the group consisting of 1D8, 3Elor, 4B4 (BioLegend 309809), H4-1BB-M127 (BD Pharmingen 552532), BBK2 (Thermo Fisher MS621PABX), 145501 (Leinco Technologies B591), the antibody produced by cell line deposited as ATCC No. HB-11248 and disclosed in U.S. Pat. No. 6,974,863, 5F4 (BioLegend 31 1503), C65-485 (BD Pharmingen 559446), antibodies disclosed in U.S. Patent Application Publication No. US 2005/0095244, antibodies disclosed in U.S. Pat. No. 7,288,638 (such as 20H4.9-IgG1 (BMS-663031), antibodies disclosed in U.S. Pat. No. 6,887,673 (such as 4E9 or BMS-554271), antibodies disclosed in U.S. Pat. No. 7,214,493, antibodies disclosed in U.S. Pat. No. 6,303,121, antibodies disclosed in U.S. Pat. No. 6,569,997, antibodies disclosed in U.S. Pat. No. 6,905,685 (such as 4E9 or BMS-554271), antibodies disclosed in U.S. Pat. No. 6,362,325 (such as 1D8 or BMS-469492; 3H3 or BMS-469497; or 3E1); antibodies disclosed in U.S. Pat. No. 6,974,863 (such as 53A2); antibodies disclosed in U.S. Pat. No. 6,210,669 (such as 1D8, 3B8, or 3E1), antibodies described in U.S. Pat. No. 5,928,893, antibodies disclosed in U.S. Pat. No. 6,303,121, antibodies disclosed in U.S. Pat. No. 6,569,997, antibodies disclosed in International Patent Application Publication Nos. WO 2012/177788, WO 2015/119923, and WO 2010/042433, and fragments, derivatives, conjugates, variants, or biosimilars thereof, wherein the disclosure of each of the foregoing patents or patent application publications is incorporated by reference here.

In some embodiments, the 4-1BB agonist is a 4-1BB agonistic fusion protein described in International Patent Application Publication Nos. WO 2008/025516 A1, WO 2009/007120 A1, WO 2010/003766 A1, WO 2010/010051 A1, and WO 2010/078966 A1; U.S. Patent Application Publication Nos. US 2011/0027218 A1, US 2015/0126709 A1, US 2011/0111494 A1, US 2015/0110734 A1, and US 2015/0126710 A1; and U.S. Pat. Nos. 9,359,420, 9,340,599, 8,921,519, and 8,450,460, the disclosures of which are incorporated by reference herein.

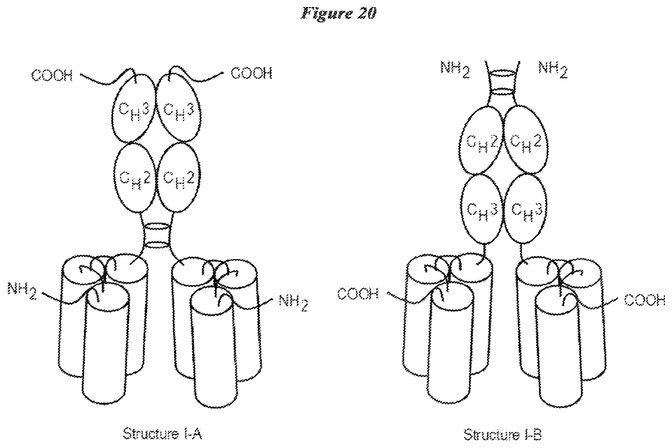

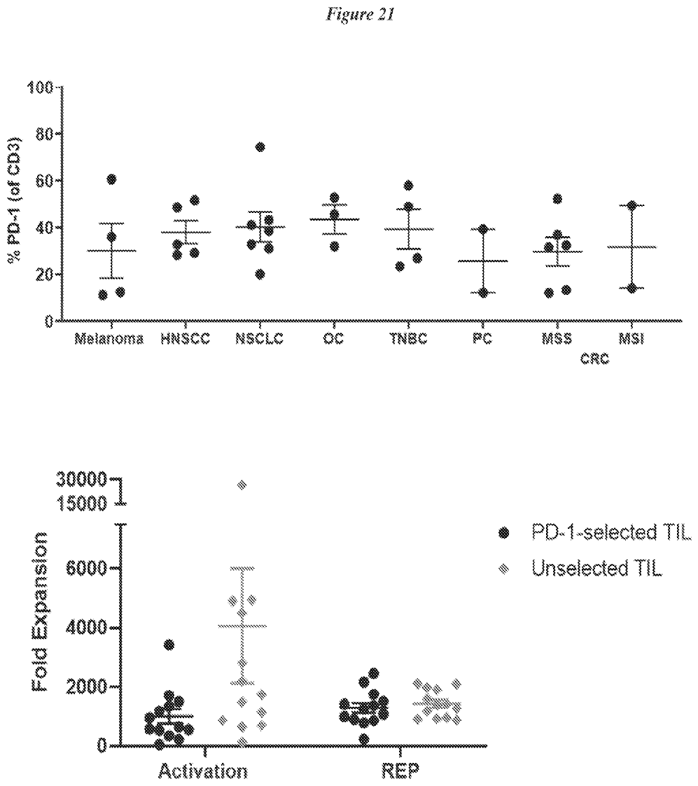

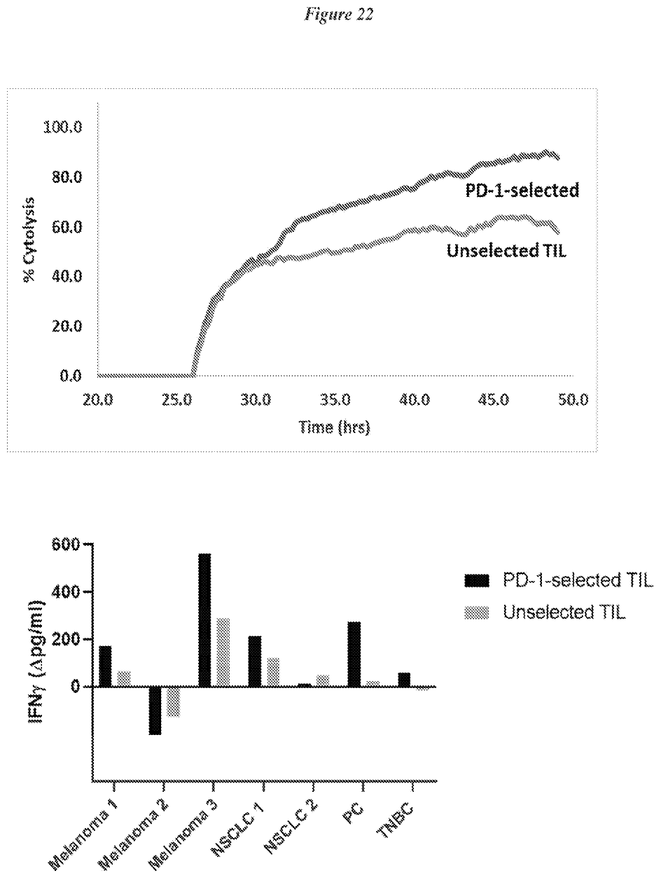

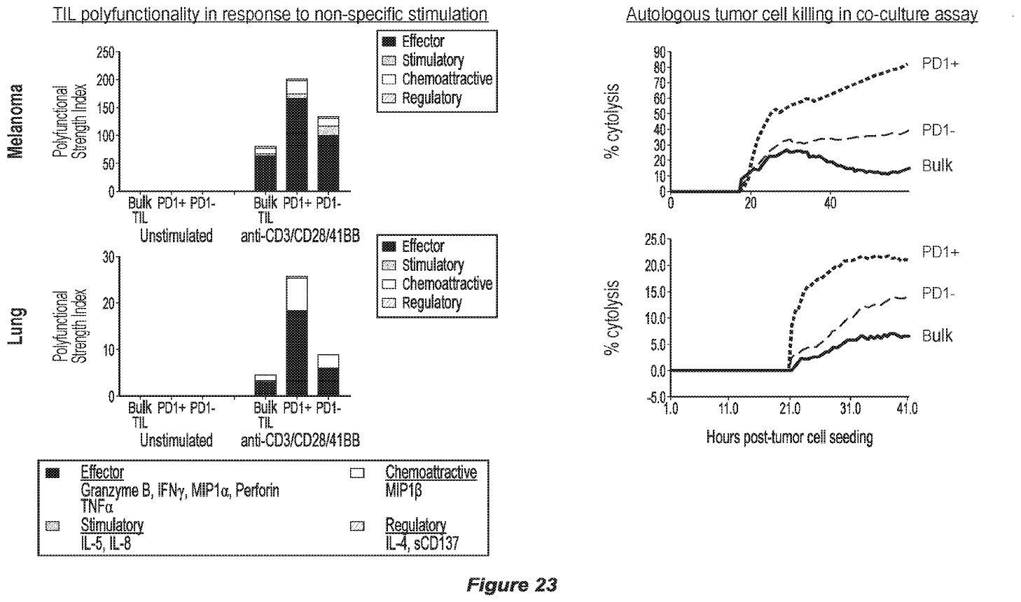

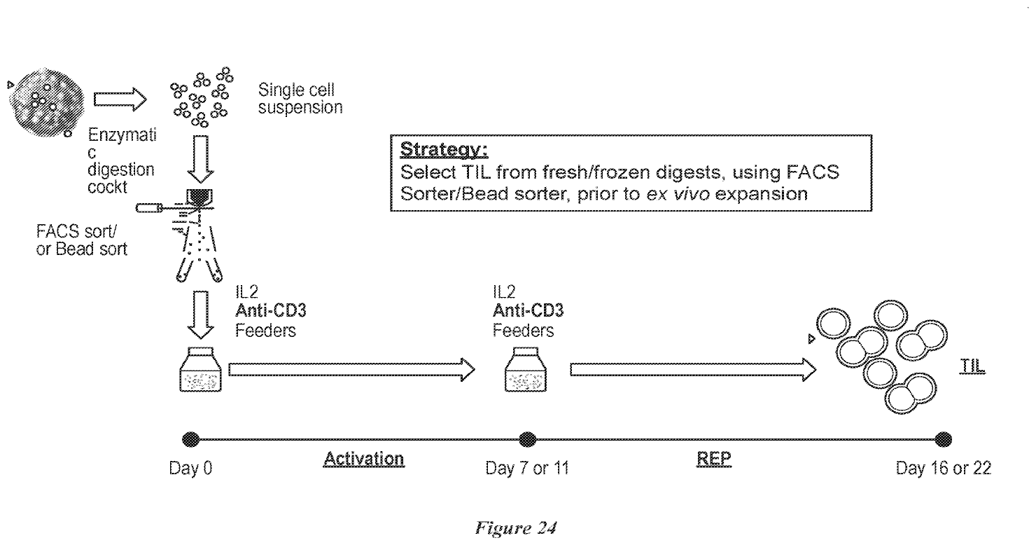

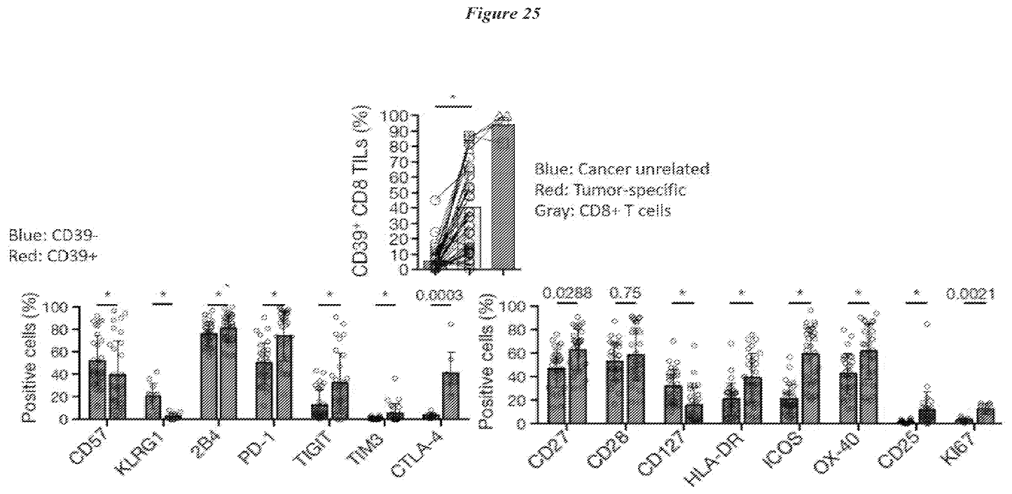

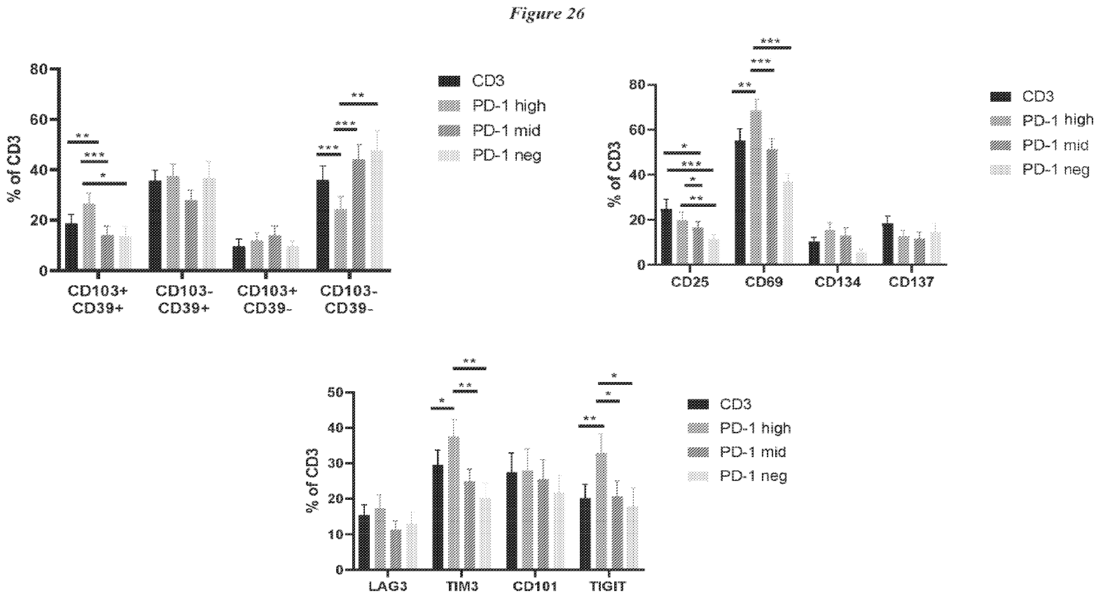

In some embodiments, the 4-1BB agonist is a 4-1BB agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof as provided in FIG. 20.

In structures I-A and I-B (see, FIG. 20), the cylinders refer to individual polypeptide binding domains. Structures I-A and I-B comprise three linearly-linked TNFRSF binding domains derived from e.g., 4-1BBL (4-1BB ligand, CD137 ligand (CD137L), or tumor necrosis factor superfamily member 9 (TNFSF9) or an antibody that binds 4-1BB, which fold to form a trivalent protein, which is then linked to a second triavelent protein through IgG1-Fc (including C_{H}3 and C_{H}2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a V_{H} and a V_{L} chain connected by a linker that may comprise hydrophilic residues and Gly and Ser sequences for flexibility, as well as Glu and Lys for solubility. Any scFv domain design may be used, such as those described in de Marco, Microbial Cell Factories, 2011, 10, 44; Ahmad, et al., Clin. & Dev. Immunol. 2012, 980250; Monnier, et al., Antibodies, 2013, 2, 193-208; or in references incorporated elsewhere herein. Fusion protein structures of this form are described in U.S. Pat. Nos. 9,359,420, 9,340,599, 8,921,519, and 8,450,460, the disclosures of which are incorporated by reference herein.

Amino acid sequences for the other polypeptide domains of structure I-A are given in Table 9. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides.

TABLE 9
Amino acid sequences for TNFRSF agonist fusion proteins, including 4-1BB agonist
fusion proteins, with C-terminal Fc-antibody fragment fusion protein design
(structure I-A).

| Identifier | Sequence (One-Letter Amino Acid Symbols) | |
|---|---|---|
| SEQ ID NO: 31 | KSCDKTHTCP PCPAPELLGG PSVFLFPPKP KDTLMISRTP EVTCVVVDVS HEDPEVKFNW | 60 |
| Fc domain | YVDGVEVHNA KTKPREEQYN STYRVVSVLT VLHQDWINGK EYKCKVSNKA LPAPIEKTIS | 120 |
| | KAKGQPREPQ VYTLPPSREE MTKNQVSLTC LVKGFYPSDI AVEWESNGQP ENNYKTTPPV | 180 |
| | LDSDGSFFLY SKLTVDKSRW CQGNVFSCSV MHEALHNHYT QKSLSLSPGK | 230 |
| SEQ ID NO: 32 | GGPGSSKSCD KTATCPPCPA PE | 22 |
| linker | | |
| SEQ ID NO: 33 | GGSGSSKSCD KTATCPPCPA PE | 22 |
| linker | | |
| SEQ ID NO: 34 | GGPGSSSSSS SKSCDKTHTC PPCPAPE | 27 |
| linker | | |
| SEQ ID NO: 35 | GGSGSSSSSS SKSCDKTHTC PPCPAPE | 27 |
| linker | | |
| SEQ ID NO: 36 | GGPGSSSSSS SSSKSCDKTH TCPPCPAPE | 29 |
| linker | | |
| SEQ ID NO: 37 | GGSGSSSSSS SSSKSCDKTH TCPPCPAPE | 29 |
| linker | | |
| SEQ ID NO: 38 | GGPGSSGSGS SDKTHTCPPC PAPE | 24 |
| linker | | |
| SEQ ID NO: 39 | GGPGSSGSGS DKTHTCPPCP APE | 23 |
| linker | | |
| SEQ ID NO: 40 | GGPSSSGSDK THTCPPCPAP E | 21 |
| linker | | |
| SEQ ID NO: 41 | GGSSSSSSSS GSDKTHTCPP CPAPE | 25 |
| linker | | |

Amino acid sequences for the other polypeptide domains of structure I-B are given in Table 10. If an Fc antibody fragment is fused to the N-terminus of an TNRFSF agonist fusion protein as in structure I-B, the sequence of the Fc module is preferably that shown in SEQ ID NO:42, and the linker sequences are preferably selected from those embodiments set forth in SED ID NO:43 to SEQ ID NO:45.

TABLE 10
Amino acid sequences for TNFRSF agonist fusion proteins, including 4-1BB agonist
fusion proteins, with N-terminal Fc-antibody fragment fusion protein design
(structure I-B).

| Identifier | Sequence (One-Letter Amino Acid Symbols) | |
|---|---|---|
| SEQ ID NO: 42 | METDTLLLWV LLLWVPAGNG DKTHTCPPCP APELLGGPSV FLFPPKPKDT LMISRTPEVT | 60 |
| Fc domain | CVVVDVSHED PEVKENWYVD GVEVHNAKTK PREEQYNSTY RVVSVLTVLH QDWINGKEYK | 120 |
| | CKVSNKALPA PIEKTISKAK GQPREPQVYT LPPSREEMTK NQVSLTCLVK GFYPSDIAVE | 180 |
| | WESNGQPENN YKTTPPVLDS DGSFFLYSKL TVDKSRWQQG NVFSCSVMHE ALHNHYTQKS | 240 |
| | LSLSPG | 246 |
| SEQ ID NO: 43 | SGSGSGSGSG S | 11 |
| linker | | |
| SEQ ID NO: 44 | SSSSSSGSGS GS | 12 |
| linker | | |
| SEQ ID NO: 45 | SSSSSSGSGS GSGSGS | 16 |
| linker | | |

In some embodiments, a 4-1BB agonist fusion protein according to structures I-A or I-B comprises one or more 4-1BB binding domains selected from the group consisting of a variable heavy chain and variable light chain of utomilumab, a variable heavy chain and variable light chain of urelumab, a variable heavy chain and variable light chain of utomilumab, a variable heavy chain and variable light chain selected from the variable heavy chains and variable light chains described in Table 11, any combination of a variable heavy chain and variable light chain of the foregoing, and fragments, derivatives, conjugates, variants, and biosimilars thereof

In some embodiments, a 4-1BB agonist fusion protein according to structures I-A or I-B comprises one or more 4-1BB binding domains comprising a 4-1BBL sequence. In some embodiments, a 4-1BB agonist fusion protein according to structures I-A or I-B comprises one or more 4-1BB binding domains comprising a sequence according to SEQ ID NO:46. In some embodiments, a 4-1BB agonist fusion protein according to structures I-A or I-B comprises one or more 4-1BB binding domains comprising a soluble 4-1BBL sequence. In some embodiments, a 4-1BB agonist fusion protein according to structures I-A or I-B comprises one or more 4-1BB binding domains comprising a sequence according to SEQ ID NO:47.

In some embodiments, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:13 and SEQ ID NO:14, respectively, wherein the V_{H} and V_{L} domains are connected by a linker. In some embodiments, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:23 and SEQ ID NO:24, respectively, wherein the V_{H} and V_{L} domains are connected by a linker. In some embodiments, a 4-1BB agonist fusion protein according to structures I-A or I-B comprises one or more 4-1BB binding domains that is a scFv domain comprising V_{H} and V_{L} regions that are each at least 95% identical to the V_{H} and V_{L} sequences given in Table 11, wherein the V_{H} and V_{L} domains are connected by a linker.

TABLE 11
Additionalpolypeptidedomainsusefulas4-1BBbindingdomainsinfusionproteinsor
asscFv4-1BBagonistantibodies.

| Identifier | Sequence(One-LetterAminoAcidSymbols) | |
|---|---|---|
| SEQ ID NO: 46 | MEYASDASLDPEAPWPPAPRARACRVLPWALVAGLLLLLLLAAACAVFLACPWAVSGARA | 60 |
| 4-1BBL | SPGSAASPRLREGPELSPDDPAGLLDLRQGMFAQLVAQNVLLIDGPLSWYSDPGLAGVSL | 120 |
| | TGGLSYKEDTKELVVAKAGVYYVFFQLELRRVVAGEGSGSVSLALHLQPIRSAAGAAALA | 180 |
| | LTVDLPPASSEARNSAFGFQGRILHLSAGQRLGVHLHTEARARHAWQLTQGATVLGLFRV | 240 |
| | TPEIPAGLPSPRSE | 254 |
| SEQ ID NO: 47 | LRQGMFAQLVAQNVLLIDGPLSWYSDPGLAGVSLTGGLSYKEDTKELVVAKAGVYYVFFQ | 60 |
| 4-1BBL soluble | LELRRVVAGEGSGSVSLALHLQPLRSAAGAAALALTVDLPPASSEARNSAFGFQGRLLHL | 120 |
| domain | SAGQRLGVHLHTEARARHAWQLTQGATVLGLFRVTPEIPAGLPSPRSE | 168 |
| SEQ ID NO: 48 | QVQLQQPGAELVKPGASVKLSCKASGYTFSSYWMHWVKQRPGQVLEWIGEINPGNGHTNY | 60 |
| variable heavy | NEKFKSKATLTVDKSSSTAYMQLSSLTSEDSAVYYCARSFTTARGFAYWGQGTLVTVS | 118 |
| chain for 4B4-1- | | |
| 1 version 1 | | |
| SEQ ID NO: 49 | DIVMTQSPATQSVTPGDRVSISCRASQTISDYLHWYQQKSHESPRLLIKYASQSISGIPS | 60 |
| variable light | RFSGSGSGSDFTLSINSVEPEDVGVYYCQDGHSFPPTFGGGTKLEIK | 107 |
| chain for 4B4-1- | | |
| 1 version 1 | | |
| SEQ ID NO: 50 | QVQLQQPGAELVKPGASVKLSCKASGYTFSSYWMHWVKQRPGQVLEWIGEINPGNGHTNY | 60 |
| variable heavy | NEKFKSKATLTVDKSSSTAYMQLSSLTSEDSAVYYCARSFTTARGFAYWGQGTLVTVSA | 119 |
| chain for 4B4-1- | | |
| 1 version 2 | | |
| SEQ ID NO: 51 | DIVMTQSPATQSVTPGDRVSISCRASQTISDYLHWYQQKSHESPRLLIKYASQSISGIPS | 60 |
| variable light | RFSGSGSGSDFTLSINSVEPEDVGVYYCQDGHSFPPTFGGGTKLEIKR | 108 |
| chain for 4B4-1- | | |
| 1 version 2 | | |
| SEQ ID NO: 52 | MDWTWRILFLVAAATGAHSEVQLVESGGGLVQPGGSLRLSCAASGFTFSDYWMSWVRQAP | 60 |
| variable heavy | GKGLEWVADIKNDGSYTNYAPSITNRFTISRDNAKNSLYLQMNSLRAEDTAVYYCARELT | 120 |
| chain for H39E3- | | |
| 2 | | |
| SEQ ID NO: 53 | MEAPAQLLFLLLLWLPDTTGDIVMTQSPDSLAVSLGERATINCKSSQSLISSGNQKNYL | 60 |
| variable light | WYQQKPGQPPKLLIYYASTRQSGVPDRFSGSGSGTDFTLTISSLQAEDVA | 110 |
| chain for H39E3- | | |
| 2 | | |

In some embodiments, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, and wherein the additional domain is a Fab or Fc fragment domain. In some embodiments, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, wherein the additional domain is a Fab or Fc fragment domain, wherein each of the soluble 4-1BB domains lacks a stalk region (which contributes to trimerisation and provides a certain distance to the cell membrane, but is not part of the 4-1BB binding domain) and the first and the second peptide linkers independently have a length of 3-8 amino acids.

In some embodiments, the 4-1BB agonist is a 4-1BB agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein each TNF superfamily cytokine domain is a 4-1BB binding domain.

In some embodiments, the 4-1BB agonist is a 4-1BB agonistic scFv antibody comprising any of the foregoing V_{H} domains linked to any of the foregoing V_{L} domains.

In some embodiments, the 4-1BB agonist is BPS Bioscience 4-1BB agonist antibody catalog no. 79097-2, commercially available from BPS Bioscience, San Diego, Calif., USA. In some embodiments, the 4-1BB agonist is Creative Biolabs 4-1BB agonist antibody catalog no. MOM-18179, commercially available from Creative Biolabs, Shirley, NY, USA.

3. OX40 (CD134) AGONISTS

In some embodiments, the TNFRSF agonist is an OX40 (CD134) agonist. The OX40 agonist may be any OX40 binding molecule known in the art. The OX40 binding molecule may be a monoclonal antibody or fusion protein capable of binding to human or mammalian OX40. The OX40 agonists or OX40 binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The OX40 agonist or OX40 binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to OX40. In some embodiments, the OX40 agonist is an antigen binding protein that is a fully human antibody. In some embodiments, the OX40 agonist is an antigen binding protein that is a humanized antibody. In some embodiments, OX40 agonists for use in the presently disclosed methods and compositions include anti-OX40 antibodies, human anti-OX40 antibodies, mouse anti-OX40 antibodies, mammalian anti-OX40 antibodies, monoclonal anti-OX40 antibodies, polyclonal anti-OX40 antibodies, chimeric anti-OX40 antibodies, anti-OX40 adnectins, anti-OX40 domain antibodies, single chain anti-OX40 fragments, heavy chain anti-OX40 fragments, light chain anti-OX40 fragments, anti-OX40 fusion proteins, and fragments, derivatives, conjugates, variants, or biosimilars thereof. In some embodiments, the OX40 agonist is an agonistic, anti-OX40 humanized or fully human monoclonal antibody (i.e., an antibody derived from a single cell line).

In some embodiments, the OX40 agonist or OX40 binding molecule may also be a fusion protein. OX40 fusion proteins comprising an Fc domain fused to OX40L are described, for example, in Sadun, et al., J. Immunother. 2009, 182, 1481-89. In some embodiments, a multimeric OX40 agonist, such as a trimeric or hexameric OX40 agonist (with three or six ligand binding domains), may induce superior receptor (OX40L) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic OX40 antibodies and fusion proteins are known to induce strong immune responses. Curti, et al., Cancer Res. 2013, 73, 7189-98. In some embodiments, the OX40 agonist is a monoclonal antibody or fusion protein that binds specifically to OX40 antigen in a manner sufficient to reduce toxicity. In some embodiments, the OX40 agonist is an agonistic OX40 monoclonal antibody or fusion protein that abrogates antibody-dependent cellular toxicity (ADCC), for example NK cell cytotoxicity. In some embodiments, the OX40 agonist is an agonistic OX40 monoclonal antibody or fusion protein that abrogates antibody-dependent cell phagocytosis (ADCP). In some embodiments, the OX40 agonist is an agonistic OX40 monoclonal antibody or fusion protein that abrogates complement-dependent cytotoxicity (CDC). In some embodiments, the OX40 agonist is an agonistic OX40 monoclonal antibody or fusion protein which abrogates Fc region functionality.

In some embodiments, the OX40 agonists are characterized by binding to human OX40 (SEQ ID NO:54) with high affinity and agonistic activity. In some embodiments, the OX40 agonist is a binding molecule that binds to human OX40 (SEQ ID NO:54). In some embodiments, the OX40 agonist is a binding molecule that binds to murine OX40 (SEQ ID NO:55). The amino acid sequences of OX40 antigen to which an OX40 agonist or binding molecule binds are summarized in Table 12.

TABLE 12
Amino acid sequences of OX40 antigens.

| Identifier | Sequence (One-Letter Amino Acid Symbols) | |
|---|---|---|
| SEQ ID NO: 54 | MCVGARRLGR GPCAALLLLG IGISTVTGLH CVGDTYPSND RCCHECRPGN GMVSRCSRSQ | 60 |
| human OX40 | NTVCRPCGPG FYNDVVSSKP CKPCTWCNLR SGSERKQLCT ATQDTVCRCR AGTQPLDSYK | 120 |
| (Homo sapiens) | PGVDCAPCPP GHFSPGDNQA CKPWTNCTLA GKHTLQPASN SSDAICEDRE PPATQPQETQ | 180 |
| | GPPARPITVQ PTEAWPRTSQ GPSTRPVEVP GGRAVAAILG LGLVLGLLGP LAILLALYLL | 240 |
| | RRDQRLPPDA HKPPGGGSFR TPIQEEQADA HSTLAKI | 277 |
| SEQ ID NO: 55 | MYVWVQQPTA LLLLGLTLGV TARRLNCVKH TYPSGHKCCR ECQPGHGMVS RCDHTRDTLC | 60 |
| murine OX40 | HPCETGFYNE AVNYDTCKQC TQCNHRSGSE LKQNCTPTQD TVCRCRPGTC PRQDSGYKLG | 120 |
| (Mus musculus) | VDCVPCPPGH FSPGNNQACK PWTNCTLSGK QTRHPASDSL DAVCEDRSLI ATLLWETQRP | 180 |
| | TFRPTTVQST TVWPRTSELP SPPTLVTPEG PAFAVLLGLG LGLLAPLTVI LALYLLRKAW | 240 |
| | RLPNTPKPCW GNSFRTPIQE EHTDAHFTLA KI | 272 |

In some embodiments, the compositions, processes and methods described include a OX40 agonist that binds human or murine OX40 with a K_{D} of about 100 pM or lower, binds human or murine OX40 with a K_{D} of about 90 pM or lower, binds human or murine OX40 with a K_{D} of about 80 pM or lower, binds human or murine OX40 with a K_{D} of about 70 pM or lower, binds human or murine OX40 with a K_{D} of about 60 pM or lower, binds human or murine OX40 with a K_{D} of about 50 pM or lower, binds human or murine OX40 with a K_{D} of about 40 pM or lower, or binds human or murine OX40 with a K_{D} of about 30 pM or lower.

In some embodiments, the compositions, processes and methods described include a OX40 agonist that binds to human or murine OX40 with a k_{assoc} of about 7.5×10⁵ 1/M·s or faster, binds to human or murine OX40 with a k_{assoc} of about 7.5×10⁵ 1/M·s or faster, binds to human or murine OX40 with a k_{assoc} of about 8×10⁵ 1/M·s or faster, binds to human or murine OX40 with a k_{assoc} of about 8.5×10⁵ 1/M·s or faster, binds to human or murine OX40 with a k_{assoc} of about 9×10⁵ 1/M·s or faster, binds to human or murine OX40 with a k_{assoc} of about 9.5×10⁵ 1/M s or faster, or binds to human or murine OX40 with a k_{assoc} of about 1×10⁶ 1/M s or faster.

In some embodiments, the compositions, processes and methods described include a OX40 agonist that binds to human or murine OX40 with a k_{dissoc} of about 2×10⁻⁵ 1/s or slower, binds to human or murine OX40 with a k_{dissoc} of about 2.1×10⁻⁵ 1/s or slower, binds to human or murine OX40 with a k_{dissoc} of about 2.2×10⁻⁵ 1/s or slower, binds to human or murine OX40 with a k_{dissoc} of about 2.3×10⁻⁵ 1/s or slower, binds to human or murine OX40 with a k_{dissoc} of about 2.4×10⁻⁵ 1/s or slower, binds to human or murine OX40 with a k_{dissoc} of about 2.5×10⁻⁵ 1/s or slower, binds to human or murine OX40 with a k_{dissoc} of about 2.6×10⁻⁵ 1/s or slower or binds to human or murine OX40 with a k_{dissoc} of about 2.7×10⁻⁵ 1/s or slower, binds to human or murine OX40 with a k_{dissoc} of about 2.8×10⁻⁵ 1/s or slower, binds to human or murine OX40 with a k_{dissoc} of about 2.9×10⁻⁵ 1/s or slower, or binds to human or murine OX40 with a k_{dissoc} of about 3×10⁻⁵ 1/s or slower.

In some embodiments, the compositions, processes and methods described include OX40 agonist that binds to human or murine OX40 with an IC₅₀ of about 10 nM or lower, binds to human or murine OX40 with an IC₅₀ of about 9 nM or lower, binds to human or murine OX40 with an IC₅₀ of about 8 nM or lower, binds to human or murine OX40 with an IC₅₀ of about 7 nM or lower, binds to human or murine OX40 with an IC₅₀ of about 6 nM or lower, binds to human or murine OX40 with an IC₅₀ of about 5 nM or lower, binds to human or murine OX40 with an IC₅₀ of about 4 nM or lower, binds to human or murine OX40 with an IC₅₀ of about 3 nM or lower, binds to human or murine OX40 with an IC₅₀ of about 2 nM or lower, or binds to human or murine OX40 with an IC₅₀ of about 1 nM or lower.

In some embodiments, the OX40 agonist is tavolixizumab, also known as MEDI0562 or MEDI-0562. Tavolixizumab is available from the MedImmune subsidiary of AstraZeneca, Inc. Tavolixizumab is immunoglobulin G1-kappa, anti-[Homo sapiens TNFRSF4 (tumor necrosis factor receptor (TNFR) superfamily member 4, OX40, CD134)], humanized and chimeric monoclonal antibody. The amino acid sequences of tavolixizumab are set forth in Table 13. Tavolixizumab comprises N-glycosylation sites at positions 301 and 301″, with fucosylated complex bi-antennary CHO-type glycans; heavy chain intrachain disulfide bridges at positions 22-95 (V_{H}-V_{L}), 148-204 (C_{H}1-C_{L}), 265-325 (C_{H}2) and 371-429 (C_{H}3) (and at positions 22″-95″, 148″-204″, 265″-325″, and 371″-429″); light chain intrachain disulfide bridges at positions 23′-88′ (V_{H}-V_{L}) and 134′-194′ (C_{H}1-C_{L}) (and at positions 23″′-88″′ and 134″′-194″′); interchain heavy chain-heavy chain disulfide bridges at positions 230-230″ and 233-233″; and interchain heavy chain-light chain disulfide bridges at 224-214′ and 224″-214′″. Current clinical trials of tavolixizumab in a variety of solid tumor indications include U.S. National Institutes of Health clinicaltrials.gov identifiers NCT02318394 and NCT02705482.

In some embodiments, a OX40 agonist comprises a heavy chain given by SEQ ID NO:56 and a light chain given by SEQ ID NO:57. In some embodiments, a OX40 agonist comprises heavy and light chains having the sequences shown in SEQ ID NO:56 and SEQ ID NO:57, respectively, or antigen binding fragments, Fab fragments, single-chain variable fragments (scFv), variants, or conjugates thereof. In some embodiments, a OX40 agonist comprises heavy and light chains that are each at least 99% identical to the sequences shown in SEQ ID NO:56 and SEQ ID NO:57, respectively. In some embodiments, a OX40 agonist comprises heavy and light chains that are each at least 98% identical to the sequences shown in SEQ ID NO:56 and SEQ ID NO:57, respectively. In some embodiments, a OX40 agonist comprises heavy and light chains that are each at least 97% identical to the sequences shown in SEQ ID NO:56 and SEQ ID NO:57, respectively. In some embodiments, a OX40 agonist comprises heavy and light chains that are each at least 96% identical to the sequences shown in SEQ ID NO:56 and SEQ ID NO:57, respectively. In some embodiments, a OX40 agonist comprises heavy and light chains that are each at least 95% identical to the sequences shown in SEQ ID NO:56 and SEQ ID NO:57, respectively.

In some embodiments, the OX40 agonist comprises the heavy and light chain CDRs or variable regions (VRs) of tavolixizumab. In some embodiments, the OX40 agonist heavy chain variable region (V_{H}) comprises the sequence shown in SEQ ID NO:58, and the OX40 agonist light chain variable region (V_{L}) comprises the sequence shown in SEQ ID NO:59, and conservative amino acid substitutions thereof. In some embodiments, a OX40 agonist comprises V_{H} and V_{L} regions that are each at least 99% identical to the sequences shown in SEQ ID NO:58 and SEQ ID NO:59, respectively. In some embodiments, a OX40 agonist comprises V_{H} and V_{L} regions that are each at least 98% identical to the sequences shown in SEQ ID NO:58 and SEQ ID NO:59, respectively. In some embodiments, a OX40 agonist comprises V_{H} and V_{L} regions that are each at least 97% identical to the sequences shown in SEQ ID NO:58 and SEQ ID NO:59, respectively. In some embodiments, a OX40 agonist comprises V_{H} and V_{L} regions that are each at least 96% identical to the sequences shown in SEQ ID NO:58 and SEQ ID NO:59, respectively. In some embodiments, a OX40 agonist comprises V_{H} and V_{L} regions that are each at least 95% identical to the sequences shown in SEQ ID NO:58 and SEQ ID NO:59, respectively. In some embodiments, an OX40 agonist comprises an scFv antibody comprising V_{H} and V_{L} regions that are each at least 99% identical to the sequences shown in SEQ ID NO:58 and SEQ ID NO:59.

In some embodiments, a OX40 agonist comprises heavy chain CDR1, CDR2 and CDR3 domains having the sequences set forth in SEQ ID NO:60, SEQ ID NO:61, and SEQ ID NO:62, respectively, and conservative amino acid substitutions thereof, and light chain CDR1, CDR2 and CDR3 domains having the sequences set forth in SEQ ID NO:63, SEQ ID NO:64, and SEQ ID NO:65, respectively, and conservative amino acid substitutions thereof.

In some embodiments, the OX40 agonist is a OX40 agonist biosimilar monoclonal antibody approved by drug regulatory authorities with reference to tavolixizumab. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tavolixizumab. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is tavolixizumab. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab. In some embodiments, the biosimilar is provided as a composition which further comprises one or more excipients, wherein the one or more excipients are the same or different to the excipients comprised in a reference medicinal product or reference biological product wherein the reference medicinal product or reference biological product is tavolixizumab.

TABLE 13
Amino acid sequences for OX40 agonist antibodies related to tavolixizumab.

| Identifier | Sequence (One-Letter Amino Acid Symbols) | |
|---|---|---|
| SEQ ID NO: 56 | QVQLQESGPG LVKPSQTLSL TCAVYGGSFS SGYWNWIRKH PGKGLEYIGY ISYNGITYHN | 60 |
| heavy chain for | PSLKSRITIN RDTSKNQYSL CLNSVTPEDT AVYYCARYKY DYDGGHAMDY WGQGTLVTVS | 120 |
| tavolixizumab | SASTKGPSVF PLAPSSKSTS GGTAALGCLV KDYFPEPVTV SWNSGALTSG VHTFPAVLQS | 130 |
| | SGLYSLSSVV TVPSSSLGTQ TYICNVNHKP SNTKVDKRVE PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWING KEYKCKVSNK ALPAPIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 57 | DIQMTQSPSS LSASVGDRVT ITCRASQDIS NYLNWYQQKP GKAPKLLIYY TSKLHSGVPS | 60 |
| light chain for | RFSGSGSGTD YTLTISSLQP EDFATYYCQQ GSALPWTFGQ GTKVEIKRTV AAPSVFIFPP | 120 |
| tavolixizumab | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 58 | QVQLQESGPG LVKPSQTLSL TCAVYGGSFS SGYWNWIRKI PGKGLEYIGY ISYNGITYHN | 60 |
| heavy chain | PSLKSRITIN RDTSKNQYSL CLNSVTPEDT AVYYCARYKY DYDGGHAMDY WGQGTLVT | 118 |
| variable region | | |
| for | | |
| tavolixizumab | | |
| SEQ ID NO: 59 | DIQMTQSPSS LSASVGDRVT ITCRASQDIS NYLNWYQQKP GKAPKLLIYY TSKLHSGVPS | 60 |
| light chain | RFSGSGSGTD YTLTISSLQP EDFATYYCQQ GSALPWTFGQ GTKVEIKR | 108 |
| variable region | | |
| for | | |
| tavolixizumab | | |
| SEQ ID NO: 60 | GSFSSGYWN | 9 |
| heavy chain CDR1 | | |
| for | | |
| tavolixizumab | | |
| SEQ ID NO: 61 | YIGYISYNGI TYH | 13 |
| heavy chain CDR2 | | |
| for | | |
| tavolixizumab | | |
| SEQ ID NO: 62 | RYKYDYDGGH AMDY | 14 |
| heavy chain CDR3 | | |
| for | | |
| tavolixizumab | | |
| SEQ ID NO: 63 | QDISNYLN | 8 |
| light chain CDR1 | | |
| for | | |
| tavolixizumab | | |
| SEQ ID NO: 64 | LLIYYTSKLH S | 11 |
| light chain CDR2 | | |
| for | | |
| tavolixizumab | | |
| SEQ ID NO: 65 | QQGSALPW | 8 |
| light chain CDR3 | | |
| for | | |
| tavolixizumab | | |

In some embodiments, the OX40 agonist is 11D4, which is a fully human antibody available from Pfizer, Inc. The preparation and properties of 11D4 are described in U.S. Pat. Nos. 7,960,515; 8,236,930; and 9,028,824, the disclosures of which are incorporated by reference herein. The amino acid sequences of 11D4 are set forth in Table 14.

In some embodiments, a OX40 agonist comprises a heavy chain given by SEQ ID NO:66 and a light chain given by SEQ ID NO:67. In some embodiments, a OX40 agonist comprises heavy and light chains having the sequences shown in SEQ ID NO:66 and SEQ ID NO:67, respectively, or antigen binding fragments, Fab fragments, single-chain variable fragments (scFv), variants, or conjugates thereof. In some embodiments, a OX40 agonist comprises heavy and light chains that are each at least 99% identical to the sequences shown in SEQ ID NO:66 and SEQ ID NO:67, respectively. In some embodiments, a OX40 agonist comprises heavy and light chains that are each at least 98% identical to the sequences shown in SEQ ID NO:66 and SEQ ID NO:67, respectively. In some embodiments, a OX40 agonist comprises heavy and light chains that are each at least 97% identical to the sequences shown in SEQ ID NO:66 and SEQ ID NO:67, respectively. In some embodiments, a OX40 agonist comprises heavy and light chains that are each at least 96% identical to the sequences shown in SEQ ID NO:66 and SEQ ID NO:67, respectively. In some embodiments, a OX40 agonist comprises heavy and light chains that are each at least 95% identical to the sequences shown in SEQ ID NO:66 and SEQ ID NO:67, respectively.

In some embodiments, the OX40 agonist comprises the heavy and light chain CDRs or variable regions (VRs) of 11D4. In some embodiments, the OX40 agonist heavy chain variable region (V_{H}) comprises the sequence shown in SEQ ID NO:68, and the OX40 agonist light chain variable region (V_{L}) comprises the sequence shown in SEQ ID NO:69, and conservative amino acid substitutions thereof. In some embodiments, a OX40 agonist comprises V_{H} and V_{L} regions that are each at least 99% identical to the sequences shown in SEQ ID NO:68 and SEQ ID NO:69, respectively. In some embodiments, a OX40 agonist comprises V_{H} and V_{L} regions that are each at least 98% identical to the sequences shown in SEQ ID NO:68 and SEQ ID NO:69, respectively. In some embodiments, a OX40 agonist comprises V_{H} and V_{L} regions that are each at least 97% identical to the sequences shown in SEQ ID NO:68 and SEQ ID NO:69, respectively. In some embodiments, a OX40 agonist comprises V_{H} and V_{L} regions that are each at least 96% identical to the sequences shown in SEQ ID NO:68 and SEQ ID NO:69, respectively. In some embodiments, a OX40 agonist comprises V_{H} and V_{L} regions that are each at least 95% identical to the sequences shown in SEQ ID NO:68 and SEQ ID NO:69, respectively.

In some embodiments, a OX40 agonist comprises heavy chain CDR1, CDR2 and CDR3 domains having the sequences set forth in SEQ ID NO:70, SEQ ID NO:71, and SEQ ID NO:72, respectively, and conservative amino acid substitutions thereof, and light chain CDR1, CDR2 and CDR3 domains having the sequences set forth in SEQ ID NO:73, SEQ ID NO:74, and SEQ ID NO:75, respectively, and conservative amino acid substitutions thereof.

In some embodiments, the OX40 agonist is a OX40 agonist biosimilar monoclonal antibody approved by drug regulatory authorities with reference to 11D4. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 11D4. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 11D4. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product wherein the reference medicinal product or reference biological product is 11D4. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4.

TABLE 14
Amino acid sequences for OX40 agonist antibodies related to 11D4.

| Identifier | Sequence (One-Letter Amino Acid Symbols) | |
|---|---|---|
| SEQ ID NO: 66 | EVQLVESGGG LVQPGGSLRL SCAASGFTFS SYSMNWVRQA PGKGLEWVSY ISSSSSTIDY | 60 |
| heavy chain for | ADSVKGRFTI SRDNAKNSLY IQMNSLRDED TAVYYCARES GWYLFDYWGC GTLVTVSSAS | 120 |
| 11D4 | TKGPSVFPLA PCSRSTSEST AALGCLVKDY FPEPVTVSWN SGALTSGVHT FPAVLQSSGL | 180 |
| | YSLSSVVTVP SSNFGTQTYT CNVDHKPSNT KVDKTVERKC CVECPPCPAP PVAGPSVFLF | 240 |
| | PPKPKDTLMI SRTPEVTCVV VDVSHEDPEV QFNWYVDGVE VHNAKTKPRE EQFNSTFRVV | 300 |
| | SVLTVVHQDW LNGKEYKCKV SNKGLPAPIE KTISKTKGQP REPQVYTLPP SREEMTKNQV | 360 |
| | SLTCLVKGFY PSDIAVEWES NGCPENNYKT TPPMLDSDGS FFLYSKLTVD KSRWQQGNVF | 420 |
| | SCSVMHEALH NHYTQKSLSL SPGK | |
| SEQ ID NO: 67 | DIQMTQSPSS LSASVGDRVT ITCRASQGIS SWLAWYQQKP EKAPKSLIYA ASSLQSGVPS | 60 |
| light chain for | RFSGSGSGTD FTLTISSLQP EDFATYYCQQ YNSYPPTFGG GTKVEIKRTV AAPSVFIFPP | 120 |
| 11D4 | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 68 | EVQLVESGGG LVQPGGSLRL SCAASGFTFS SYSMNWVRQA PGKGLEWVSY ISSSSSTIDY | 60 |
| heavy chain | ADSVKGRFTI SRDNAKNSLY IQMNSLRDED TAVYYCARES GWYLFDYWGç GTLVTVSS | 118 |
| variable region | | |
| for 11D4 | | |
| SEQ ID NO: 69 | DIQMTQSPSS LSASVGDRVT ITCRASQGIS SWLAWYQQKP EKAPKSLIYA ASSLQSGVPS | 60 |
| light chain | RFSGSGSGTD FTLTISSLQP EDFATYYCQQ YNSYPPTFGG GTKVEIK | 107 |
| variable region | | |
| for 11D4 | | |
| SEQ ID NO: 70 | SYSMN | 5 |
| heavy chain CDR1 | | |
| for 11D4 | | |
| SEQ ID NO: 71 | YISSSSSTID YADSVKG | 17 |
| heavy chain CDR2 | | |
| for 11D4 | | |
| SEQ ID NO: 72 | ESGWYLFDY | 9 |
| heavy chain CDR3 | | |
| for 11D4 | | |
| SEQ ID NO: 73 | RASQGISSWL A | 11 |
| light chain CDR1 | | |
| for 11D4 | | |
| SEQ ID NO: 74 | AASSLQS | 7 |
| light chain CDR2 | | |
| for 11D4 | | |
| SEQ ID NO: 75 | QQYNSYPPT | 9 |
| light chain CDR3 | | |
| for 11D4 | | |

In some embodiments, the OX40 agonist is 18D8, which is a fully human antibody available from Pfizer, Inc. The preparation and properties of 18D8 are described in U.S. Pat. Nos. 7,960,515; 8,236,930; and 9,028,824, the disclosures of which are incorporated by reference herein. The amino acid sequences of 18D8 are set forth in Table 15.

In some embodiments, a OX40 agonist comprises a heavy chain given by SEQ ID NO:76 and a light chain given by SEQ ID NO:77. In some embodiments, a OX40 agonist comprises heavy and light chains having the sequences shown in SEQ ID NO:76 and SEQ ID NO:77, respectively, or antigen binding fragments, Fab fragments, single-chain variable fragments (scFv), variants, or conjugates thereof. In some embodiments, a OX40 agonist comprises heavy and light chains that are each at least 99% identical to the sequences shown in SEQ ID NO:76 and SEQ ID NO:77, respectively. In some embodiments, a OX40 agonist comprises heavy and light chains that are each at least 98% identical to the sequences shown in SEQ ID NO:76 and SEQ ID NO:77, respectively. In some embodiments, a OX40 agonist comprises heavy and light chains that are each at least 97% identical to the sequences shown in SEQ ID NO:76 and SEQ ID NO:77, respectively. In some embodiments, a OX40 agonist comprises heavy and light chains that are each at least 96% identical to the sequences shown in SEQ ID NO:76 and SEQ ID NO:77, respectively. In some embodiments, a OX40 agonist comprises heavy and light chains that are each at least 95% identical to the sequences shown in SEQ ID NO:76 and SEQ ID NO:77, respectively.

In some embodiments, the OX40 agonist comprises the heavy and light chain CDRs or variable regions (VRs) of 18D8. In some embodiments, the OX40 agonist heavy chain variable region (V_{H}) comprises the sequence shown in SEQ ID NO:78, and the OX40 agonist light chain variable region (V_{L}) comprises the sequence shown in SEQ ID NO:79, and conservative amino acid substitutions thereof. In some embodiments, a OX40 agonist comprises V_{H} and V_{L} regions that are each at least 99% identical to the sequences shown in SEQ ID NO:78 and SEQ ID NO:79, respectively. In some embodiments, a OX40 agonist comprises V_{H} and V_{L} regions that are each at least 98% identical to the sequences shown in SEQ ID NO:78 and SEQ ID NO:79, respectively. In some embodiments, a OX40 agonist comprises V_{H} and V_{L} regions that are each at least 97% identical to the sequences shown in SEQ ID NO:78 and SEQ ID NO:79, respectively. In some embodiments, a OX40 agonist comprises V_{H} and V_{L} regions that are each at least 96% identical to the sequences shown in SEQ ID NO:78 and SEQ ID NO:79, respectively. In some embodiments, a OX40 agonist comprises V_{H} and V_{L} regions that are each at least 95% identical to the sequences shown in SEQ ID NO:78 and SEQ ID NO:79, respectively.

In some embodiments, a OX40 agonist comprises heavy chain CDR1, CDR2 and CDR3 domains having the sequences set forth in SEQ ID NO:80, SEQ ID NO:81, and SEQ ID NO:82, respectively, and conservative amino acid substitutions thereof, and light chain CDR1, CDR2 and CDR3 domains having the sequences set forth in SEQ ID NO:83, SEQ ID NO:84, and SEQ ID NO:85, respectively, and conservative amino acid substitutions thereof.

In some embodiments, the OX40 agonist is a OX40 agonist biosimilar monoclonal antibody approved by drug regulatory authorities with reference to 18D8. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 18D8. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 18D8. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product wherein the reference medicinal product or reference biological product is 18D8. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8.

TABLE 15
Amino acid sequences for OX40 agonist antibodies related to 18D8.

| Identifier | Sequence (One-Letter Amino Acid Symbols) | |
|---|---|---|
| SEQ ID NO: 76 | EVQLVESGGG LVQPGRSLRL SCAASGFTFD DYAMHWVRQA PGKGLEWVSG ISWNSGSIGY | 60 |
| heavy chain for | ADSVKGRFTI SRDNAKNSLY IQMNSLRAED TALYYCAKDQ STADYYFYYG MDVWGQGTTV | 120 |
| 18D8 | TVSSASTKGP SVFPLAPCSR STSESTAALG CLVKDYFPEP VTVSWNSGAL TSGVHTFPAV | 180 |
| | LQSSGLYSLS SVVTVPSSNF GTCTYTCNVD HKPSNTKVDK TVERKCCVEC PPCPAPPVAG | 240 |
| | PSVFLFPPKP KDTLMISRTP EVTCVVVDVS HEDPEVQFNW YVDGVEVHNA KTKPREEQFN | 300 |
| | STFRVVSVLT VVHQDWLNGK EYKCKVSNKG LPAPIEKTIS KTKGQPREPç VYTLPPSREE | 360 |
| | MTKNQVSLTC LVKGFYPSDI AVEWESNGQP ENNYKTTPPM LDSDGSFFLY SKLTVDKSRW | 420 |
| | QQGNVFSCSV MHEALHNHYT CKSLSLSPGK | 450 |
| SEQ ID NO: 77 | EIVVTQSPAT LSLSPGERAT ISCRASQSVS SYLAWYQQKP GQAPRLLIYD ASNRATGIPA | 60 |
| light chain for | RFSGSGSGTD FTLTISSLEP EDFAVYYCQQ RSNWPTFGQG TKVEIKRTVA APSVFIFPPS | 120 |
| 18D8 | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 78 | EVQLVESGGG LVQPGRSLRL SCAASGFTFD DYAMHWVRQA PGKGLEWVSG ISWNSGSIGY | 60 |
| for 18D8 | ADSVKGRFTI SRDNAKNSLY IQMNSLRAED TALYYCAKDQ STADYYFYYG MDVWGQGTTV | 120 |
| heavy chain | TVSS | 124 |
| variable region | | |
| SEQ ID NO: 79 | EIVVTQSPAT LSLSPGERAT ISCRASQSVS SYLAWYQQKP GQAPRLLIYD ASNRATGIPA | 60 |
| light chain | RFSGSGSGTD FTLTISSLEP EDFAVYYCQQ RSNWPTFGQG TKVEIK | 106 |
| variable region | | |
| for 18D8 | | |
| SEQ ID NO: 80 | DYAMH | 5 |
| heavy chain CDR1 | | |
| for 18D8 | | |
| SEQ ID NO: 81 | GISWNSGSIG YADSVKG | 17 |
| heavy chain CDR2 | | |
| for 18D8 | | |
| SEQ ID NO: 82 | DQSTADYYFY YGMDV | 15 |
| heavy chain CDR3 | | |
| for 18D8 | | |
| SEQ ID NO: 83 | RASQSVSSYL A | 11 |
| light chain CDR1 | | |
| for 18D8 | | |
| SEQ ID NO: 84 | DASNRAT | 7 |
| light chain CDR2 | | |
| for 18D8 | | |
| SEQ ID NO: 85 | QQRSNWPT | 8 |
| light chain CDR3 | | |
| for 18D8 | | |

In some embodiments, the OX40 agonist is Hu119-122, which is a humanized antibody available from GlaxoSmithKline plc. The preparation and properties of Hu119-122 are described in U.S. Pat. Nos. 9,006,399 and 9,163,085, and in International Patent Publication No. WO 2012/027328, the disclosures of which are incorporated by reference herein. The amino acid sequences of Hu119-122 are set forth in Table 16.

In some embodiments, the OX40 agonist comprises the heavy and light chain CDRs or variable regions (VRs) of Hu119-122. In some embodiments, the OX40 agonist heavy chain variable region (V_{H}) comprises the sequence shown in SEQ ID NO:86, and the OX40 agonist light chain variable region (V_{L}) comprises the sequence shown in SEQ ID NO:87, and conservative amino acid substitutions thereof. In some embodiments, a OX40 agonist comprises V_{H} and V_{L} regions that are each at least 99% identical to the sequences shown in SEQ ID NO:86 and SEQ ID NO:87, respectively. In some embodiments, a OX40 agonist comprises V_{H} and V_{L} regions that are each at least 98% identical to the sequences shown in SEQ ID NO:86 and SEQ ID NO:87, respectively. In some embodiments, a OX40 agonist comprises V_{H} and V_{L} regions that are each at least 97% identical to the sequences shown in SEQ ID NO:86 and SEQ ID NO:87, respectively. In some embodiments, a OX40 agonist comprises V_{H} and V_{L} regions that are each at least 96% identical to the sequences shown in SEQ ID NO:86 and SEQ ID NO:87, respectively. In some embodiments, a OX40 agonist comprises V_{H} and V_{L} regions that are each at least 95% identical to the sequences shown in SEQ ID NO:86 and SEQ ID NO:87, respectively.

In some embodiments, a OX40 agonist comprises heavy chain CDR1, CDR2 and CDR3 domains having the sequences set forth in SEQ ID NO:88, SEQ ID NO:89, and SEQ ID NO:90, respectively, and conservative amino acid substitutions thereof, and light chain CDR1, CDR2 and CDR3 domains having the sequences set forth in SEQ ID NO:91, SEQ ID NO:92, and SEQ ID NO:93, respectively, and conservative amino acid substitutions thereof.

In some embodiments, the OX40 agonist is a OX40 agonist biosimilar monoclonal antibody approved by drug regulatory authorities with reference to Hu119-122. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu119-122. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu119-122. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122. In some embodiments, the biosimilar is provided as a composition which further comprises one or more excipients, wherein the one or more excipients are the same or different to the excipients comprised in a reference medicinal product or reference biological product; wherein the reference medicinal product or reference biological product is Hu119-122.

TABLE 16
Amino acid sequences for OX40 agonist antibodies related to Hul 19-122.

| Identifier | Sequence (One-Letter Amino Acid Symbols) | |
|---|---|---|
| SEQ ID NO: 86 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY IQMNSLRAED TAVYYCARHY DDYYAWFAYW GQGTMVTVSS | 120 |
| variable region | | |
| for Hu119-122 | | |
| SEQ ID NO: 87 | EIVLTQSPAT LSLSPGERAT ISCRASKSVS TSGYSYMHWY QQKPGQAPRI LIYLASNLES | 60 |
| light chain | GVPARFSGSG SGTDFTLTIS SLEPEDFAVY YCQHSRELPL TFGGGTKVEI K | 111 |
| variable region | | |
| for Hu119-122 | | |
| SEQ ID NO: 88 | SHDMS | 5 |
| heavy chain CDR1 | | |
| for Hu119-122 | | |
| SEQ ID NO: 89 | AINSDGGSTY YPDTMER | 17 |
| heavy chain CDR2 | | |
| for Hul19-122 | | |
| SEQ ID NO: 90 | HYDDYYAWFA Y | 11 |
| heavy chain CDR3 | | |
| for Hu119-122 | | |
| SEQ ID NO: 91 | RASKSVSTSG YSYMH | 15 |
| light chain CDR1 | | |
| for Hu119-122 | | |
| SEQ ID NO: 92 | LASNLES | 7 |
| light chain CDR2 | | |
| for Hu119-122 | | |
| SEQ ID NO: 93 | QHSRELPLT | 9 |
| light chain CDR3 | | |
| for Hu119-122 | | |

In some embodiments, the OX40 agonist is Hu106-222, which is a humanized antibody available from GlaxoSmithKline plc. The preparation and properties of Hu106-222 are described in U.S. Pat. Nos. 9,006,399 and 9,163,085, and in International Patent Publication No. WO 2012/027328, the disclosures of which are incorporated by reference herein. The amino acid sequences of Hu106-222 are set forth in Table 17.

In some embodiments, the OX40 agonist comprises the heavy and light chain CDRs or variable regions (VRs) of Hu106-222. In some embodiments, the OX40 agonist heavy chain variable region (V_{H}) comprises the sequence shown in SEQ ID NO:94, and the OX40 agonist light chain variable region (V_{L}) comprises the sequence shown in SEQ ID NO:95, and conservative amino acid substitutions thereof. In some embodiments, a OX40 agonist comprises V_{H} and V_{L} regions that are each at least 99% identical to the sequences shown in SEQ ID NO:94 and SEQ ID NO:95, respectively. In some embodiments, a OX40 agonist comprises V_{H} and V_{L} regions that are each at least 98% identical to the sequences shown in SEQ ID NO:94 and SEQ ID NO:95, respectively. In some embodiments, a OX40 agonist comprises V_{H} and V_{L} regions that are each at least 97% identical to the sequences shown in SEQ ID NO:94 and SEQ ID NO:95, respectively. In some embodiments, a OX40 agonist comprises V_{H} and V_{L} regions that are each at least 96% identical to the sequences shown in SEQ ID NO:94 and SEQ ID NO:95, respectively. In some embodiments, a OX40 agonist comprises V_{H} and V_{L} regions that are each at least 95% identical to the sequences shown in SEQ ID NO:94 and SEQ ID NO:95, respectively.

In some embodiments, a OX40 agonist comprises heavy chain CDR1, CDR2 and CDR3 domains having the sequences set forth in SEQ ID NO:96, SEQ ID NO:97, and SEQ ID NO:98, respectively, and conservative amino acid substitutions thereof, and light chain CDR1, CDR2 and CDR3 domains having the sequences set forth in SEQ ID NO:99, SEQ ID NO:100, and SEQ ID NO:101, respectively, and conservative amino acid substitutions thereof.

In some embodiments, the OX40 agonist is a OX40 agonist biosimilar monoclonal antibody approved by drug regulatory authorities with reference to Hu106-222. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu106-222. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu106-222. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222.

TABLE 17
Amino acid sequences for OX40 agonist antibodies related to Hu106-222.

| Identifier | Sequence (One-Letter Amino Acid Symbols) | |
|---|---|---|
| SEQ ID NO: 94 | QVQLVQSGSE LKKPGASVKV SCKASGYTFT DYSMHWVRQA PGAGLKWMGW INTETGEPTY | 60 |
| heavy chain | ADDFKGRFVF SLDTSVSTAY IQISSLKAED TAVYYCANPY YDYVSYYAMD YWGQGTTVTV | 120 |
| variable region | SS | 122 |
| for Hu106-222 | | |
| SEQ ID NO: 95 | DIQMTQSPSS LSASVGDRVT ITCKASQDVS TAVAWYQQKP GKAPKLLIYS ASYLYTGVPS | 60 |
| light chain | RFSGSGSGTD FTFTISSLQP EDIATYYCQQ HYSTPRTFGQ GTKLEIK | 107 |
| variable region | | |
| for Hu106-222 | | |
| SEQ ID NO: 96 | DYSMH | 5 |
| heavy chain CDR1 | | |
| for Hu106-222 | | |
| SEQ ID NO: 97 | WINTETGEPT YADDFKG | 17 |
| heavy chain CDR2 | | |
| for Hu106-222 | | |
| SEQ ID NO: 98 | PYYDYVSYYA MDY | 13 |
| heavy chain CDR3 | | |
| for Hu106-222 | | |
| SEQ ID NO: 99 | KASQDVSTAV A | 11 |
| light chain CDR1 | | |
| for Hu106-222 | | |
| SEQ ID NO: 100 | SASYLYT | 7 |
| light chain CDR2 | | |
| for Hu106-222 | | |
| SEQ ID NO: 101 | QQHYSTPRT | 9 |
| light chain CDR3 | | |
| for Hu106-222 | | |

In some embodiments, the OX40 agonist antibody is MEDI6469 (also referred to as 9B12). MEDI6469 is a murine monoclonal antibody. Weinberg, et al., J Immunother. 2006, 29, 575-585. In some embodiments the OX40 agonist is an antibody produced by the 9B12 hybridoma, deposited with Biovest Inc. (Malvern, MA, USA), as described in Weinberg, et al., J. Immunother. 2006, 29, 575-585, the disclosure of which is hereby incorporated by reference in its entirety. In some embodiments, the antibody comprises the CDR sequences of MEDI6469. In some embodiments, the antibody comprises a heavy chain variable region sequence and/or a light chain variable region sequence of MEDI6469.

In some embodiments, the OX40 agonist is L106 BD (Pharmingen Product #340420). In some embodiments, the OX40 agonist comprises the CDRs of antibody L106 (BD Pharmingen Product #340420). In some embodiments, the OX40 agonist comprises a heavy chain variable region sequence and/or a light chain variable region sequence of antibody L106 (BD Pharmingen Product #340420). In some embodiments, the OX40 agonist is ACT35 (Santa Cruz Biotechnology, Catalog #20073). In some embodiments, the OX40 agonist comprises the CDRs of antibody ACT35 (Santa Cruz Biotechnology, Catalog #20073). In some embodiments, the OX40 agonist comprises a heavy chain variable region sequence and/or a light chain variable region sequence of antibody ACT35 (Santa Cruz Biotechnology, Catalog #20073). In some embodiments, the OX40 agonist is the murine monoclonal antibody anti-mCD134/mOX40 (clone OX86), commercially available from InVivoMAb, BioXcell Inc, West Lebanon, NH.

In some embodiments, the OX40 agonist is selected from the OX40 agonists described in International Patent Application Publication Nos. WO 95/12673, WO 95/21925, WO 2006/121810, WO 2012/027328, WO 2013/028231, WO 2013/038191, and WO 2014/148895; European Patent Application EP 0672141; U.S. Patent Application Publication Nos. US 2010/136030, US 2014/377284, US 2015/190506, and US 2015/132288 (including clones 20E5 and 12H3); and U.S. Pat. Nos. 7,504,101, 7,550,140, 7,622,444, 7,696,175, 7,960,515, 7,961,515, 8,133,983, 9,006,399, and 9,163,085, the disclosure of each of which is incorporated herein by reference in its entirety.

In some embodiments, the OX40 agonist is an OX40 agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A are given in Table 9. The Fc domain preferably comprises a complete constant domain (amino acids 17-230 of SEQ ID NO:31) the complete hinge domain (amino acids 1-16 of SEQ ID NO:31) or a portion of the hinge domain (e.g., amino acids 4-16 of SEQ ID NO:31). Preferred linkers for connecting a C-terminal Fc-antibody may be selected from the embodiments given in SEQ ID NO:32 to SEQ ID NO:41, including linkers suitable for fusion of additional polypeptides. Likewise, amino acid sequences for the polypeptide domains of structure I-B are given in Table 10. If an Fc antibody fragment is fused to the N-terminus of an TNRFSF fusion protein as in structure I-B, the sequence of the Fc module is preferably that shown in SEQ ID NO:42, and the linker sequences are preferably selected from those embodiments set forth in SED ID NO:43 to SEQ ID NO:45.

In some embodiments, an OX40 agonist fusion protein according to structures I-A or I-B comprises one or more OX40 binding domains selected from the group consisting of a variable heavy chain and variable light chain of tavolixizumab, a variable heavy chain and variable light chain of 11D4, a variable heavy chain and variable light chain of 18D8, a variable heavy chain and variable light chain of Hu119-122, a variable heavy chain and variable light chain of Hu106-222, a variable heavy chain and variable light chain selected from the variable heavy chains and variable light chains described in Table 18, any combination of a variable heavy chain and variable light chain of the foregoing, and fragments, derivatives, conjugates, variants, and biosimilars thereof.

In some embodiments, an OX40 agonist fusion protein according to structures I-A or I-B comprises one or more OX40 binding domains comprising an OX40L sequence. In some embodiments, an OX40 agonist fusion protein according to structures I-A or I-B comprises one or more OX40 binding domains comprising a sequence according to SEQ ID NO:102. In some embodiments, an OX40 agonist fusion protein according to structures I-A or I-B comprises one or more OX40 binding domains comprising a soluble OX40L sequence. In some embodiments, a OX40 agonist fusion protein according to structures I-A or I-B comprises one or more OX40 binding domains comprising a sequence according to SEQ ID NO:103. In some embodiments, a OX40 agonist fusion protein according to structures I-A or I-B comprises one or more OX40 binding domains comprising a sequence according to SEQ ID NO:104.

In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:58 and SEQ ID NO:59, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:68 and SEQ ID NO:69, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:78 and SEQ ID NO:79, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:86 and SEQ ID NO:87, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:94 and SEQ ID NO:95, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the V_{H} and V_{L} sequences given in Table 18, wherein the V_{H} and V_{L} domains are connected by a linker.

TABLE 18
Additional polypeptide domains useful as OX40 binding domains in fusion proteins
(e.g., structures I-A and I-B) or as scFv OX40 agonist antibodies.

| Identifier | Sequence (One-Letter Amino Acid Symbols) | |
|---|---|---|
| SEQ ID NO: 102 | MERVQPLEEN VGNAARPRFE RNKLLLVASV IQGLGLLLCF TYICLHFSAL QVSHRYPRIQ | 60 |
| OX40L | SIKVQFTEYK KEKGFILTSQ KEDEIMKVQN NSVIINCDGF YLISLKGYFS QEVNISLHYQ | 120 |
| | KDEEPLFQLK KVRSVNSLMV ASITYKDKVY LNVTTDNTSL DDFHVNGGEL ILIHQNPGEF | 180 |
| | CVL | 183 |
| SEQ ID NO: 103 | SHRYPRIQSI KVQFTEYKKE KGFILTSQKE DEIMKVQNNS VIINCDGFYL ISLKGYFSQE | 60 |
| OX40L soluble | VNISLHYQKD EEPLFQLKKV RSVNSLMVAS LTYKDKVYLN VTTDNTSLDD FHVNGGELIL | 120 |
| domain | IHQNPGEFCV L | 131 |
| SEQ ID NO: 104 | YPRIQSIKVQ FTEYKKEKGF ILTSQKEDEI MKVQNNSVII NCDGFYLISI KGYFSQEVNI | 60 |
| OX40L soluble | SLHYQKDEEP LFQLKKVRSV NSIMVASLTY KDKVYLNVTT DNTSLDDFHV NGGELILIHQ | 120 |
| domain | NPGEFCVL | 128 |
| (alternative) | | |
| SEQ ID NO: 105 | EVQLVESGGG LVQPGGSLRL SCAASGFTFS NYTMNWVRQA PGKGLEWVSA ISGSGGSTYY | 60 |
| variable heavy | ADSVKGRFTI SRDNSKNTLY IQMNSLRAED TAVYYCAKDR YSQVHYALDY WGQGTLVTVS | 120 |
| chain for 008 | | |
| SEQ ID NO: 106 | DIVMTQSPDS LPVTPGEPAS ISCRSSQSLL HSNGYNYLDW YLQKAGQSPç LLIYLGSNRA | 60 |
| variable light | SGVPDRFSGS GSGTDFTLKI SRVEAEDVGV YYCQQYYNHP TTFGQGTK | 108 |
| chain for 008 | | |
| SEQ ID NO: 107 | EVQLVESGGG VVQPGRSLRL SCAASGFTFS DYTMNWVRQA PGKGLEWVSS ISGGSTYYAD | 60 |
| variable heavy | SRKGRFTISR DNSKNTLYLQ MNNLRAEDTA VYYCARDRYF RQQNAFDYWG QGTLVTVSSA | 120 |
| chain for 011 | | |
| SEQ ID NO: 108 | DIVMTQSPDS LPVTPGEPAS ISCRSSQSLL HSNGYNYLDW YLQKAGQSPQ LLIYLGSNRA | 60 |
| variable light | SGVPDRFSGS GSGTDFTLKI SRVEAEDVGV YYCQQYYNHP TTFGQGTK | 108 |
| chain for 011 | | |
| SEQ ID NO: 109 | EVQLVESGGG LVQPRGSLRL SCAASGFTFS SYAMNWVRQA PGKGLEWVAV ISYDGSNKYY | 60 |
| variable heavy | ADSVKGRFTI SRDNSKNTLY IQMNSLRAED TAVYYCAKDR YITLPNALDY WGQGTLVTVS | 120 |
| chain for 021 | | |
| SEQ ID NO: 110 | DIQMTQSPVS LPVTPGEPAS ISCRSSQSLL HSNGYNYLDW YLQKPGQSPC LLIYLGSNRA | 60 |
| variable light | SGVPDRFSGS GSGTDFTLKI SRVEAEDVGV YYCQQYKSNP PTFGQGTK | 108 |
| chain for 021 | | |
| SEQ ID NO: 111 | EVQLVESGGG LVIPGGSLRL SCAGSGFTFS SYAMHWVRQA PGKGLEWVSA IGTGGGTYYA | 60 |
| variable heavy | DSVMGRFTIS RDNSKNTLYL CMNSLRAEDT AVYYCARYDN VMGLYWFDYW GQGTLVTVSS | 120 |
| chain for 023 | | |
| SEQ ID NO: 112 | EIVLTQSPAT LSLSPGERAT ISCRASQSVS SYLAWYQQKP GQAPRLLIYD ASNRATGIPA | 60 |
| variable light | RFSGSGSGTD FTLTISSLEP EDFAVYYCQQ RSNWPPAFGG GTKVEIKR | 108 |
| chain for 023 | | |
| SEQ ID NO: 113 | EVQLQQSGPE LVKPGASVKM SCKASGYTFT SYVMHWVKQK PGQGLEWIGY INPYNDGTKY | 60 |
| heavy chain | NEKFKGKATL TSDKSSSTAY MELSSLTSED SAVYYCANYY GSSLSMDYWG QGTSVTVSS | 119 |
| variable region | | |
| SEQ ID NO: 114 | DIQMTQTTSS LSASLGDRVT ISCRASQDIS NYLNWYQQKP DGTVKLLIYY TSRLHSGVPS | 60 |
| light chain | RFSGSGSGTD YSLTISNLEQ EDIATYFCQQ GNTLPWTFGG GTKLEIKR | 108 |
| variable region | | |
| SEQ ID NO: 115 | EVQLQQSGPE LVKPGASVKI SCKTSGYTFK DYTMHWVKQS HGKSLEWIGG IYPNNGGSTY | 60 |
| heavy chain | NQNFKDKATL TVDKSSSTAY MEFRSLTSED SAVYYCARMG YHGPHLDFDV WGAGTTVTVS | 120 |
| variable region | P | 121 |
| SEQ ID NO: 116 | DIVMTQSHKF MSTSLGDRVS ITCKASQDVG AAVAWYQQKP GQSPKLLIYW ASTRHTGVPD | 60 |
| light chain | RFTGGGSGTD FTLTISNVQS EDITDYFCQQ YINYPLTFGG GTKLEIKR | 108 |
| variable region | | |
| SEQ ID NO: 117 | QIQLVQSGPE LKKPGETVKI SCKASGYTFT DYSMHWVKQA PGKGLKWMGW INTETGEPTY | 60 |
| heavy chain | ADDFKGRFAF SLETSASTAY LQINNLKNED TATYFCANPY YDYVSYYAMD YWGHGTSVTV | 120 |
| variable region | SS | 122 |
| of humanized | | |
| antibody | | |
| SEQ ID NO: 118 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSLKAED TAVYYCANPY YDYVSYYAMD YWGQGTTVTV | 120 |
| variable region | SS | 122 |
| of humanized | | |
| antibody | | |
| SEQ ID NO: 119 | DIVMTQSHKF MSTSVRDRVS ITCKASQDVS TAVAWYQQKP GQSPKLLIYS ASYLYTGVPD | 60 |
| variable region | RFTGSGSGTD FTFTISSVQA EDLAVYYCQQ HYSTPRTFGG GTKLEIK | 107 |
| of humanized | | |
| antibody | | |
| light chain | | |
| SEQ ID NO: 120 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPRTFGG GTKLEIK | 107 |
| variable region | | |
| of humanized | | |
| antibody | | |
| SEQ ID NO: 121 | EVQLVESGGG LVQPGESLKL SCESNEYEFP SHDMSWVRKT PEKRLELVAA INSDGGSTYY | 60 |
| heavy chain | PDTMERRFII SRDNTKKTLY LQMSSLRSED TALYYCARHY DDYYAWFAYW GQGTLVTVSA | 120 |
| variable region | | |
| of humanized | | |
| antibody | | |
| SEQ ID NO: 122 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY IQMNSLRAED TAVYYCARHY DDYYAWFAYW GQGTMVTVSS | 120 |
| variable region | | |
| of humanized | | |
| antibody | | |
| SEQ ID NO: 123 | DIVLTQSPAS LAVSLGQRAT ISCRASKSVS TSGYSYMHWY QQKPGQPPKI LIYLASNLES | 60 |
| light chain | GVPARFSGSG SGTDFTLNIH PVEEEDAATY YCQHSRELPL TFGAGTKLEL K | 111 |
| variable region | | |
| of humanized | | |
| antibody | | |
| SEQ ID NO: 124 | EIVLTQSPAT LSLSPGERAT ISCRASKSVS TSGYSYMHWY QQKPGQAPRI LIYLASNLES | 60 |
| light chain | GVPARFSGSG SGTDFTLTIS SLEPEDFAVY YCQHSRELPL TFGGGTKVEI K | 111 |
| variable region | | |
| of humanized | | |
| antibody | | |
| SEQ ID NO: 125 | MYLGLNYVFI VFLLNGVQSE VKLEESGGGL VQPGGSMKLS CAASGFTFSD AWMDWVRQSP | 60 |
| heavy chain | EKGLEWVAEI RSKANNHATY YAESVNGRFT ISRDDSKSSV YLQMNSLRAE DTGIYYCTWG | 120 |
| variable region | EVFYFDYWGQ GTTLTVSS | 138 |
| SEQ ID NO: 126 | MRPSIQFLGL LLEWLHGAQC DICMTQSPSS LSASLGGKVT ITCKSSQDIN KYIAWYQHKP | 60 |
| light chain | GKGPRLLIHY TSTLQPGIPS RFSGSGSGRD YSFSISNLEP EDIATYYCLç YDNLLTFGAG | 120 |
| variable region | TKLELK | 126 |

In some embodiments, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain is a Fab or Fc fragment domain. In some embodiments, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, wherein the additional domain is a Fab or Fc fragment domain wherein each of the soluble OX40 binding domains lacks a stalk region (which contributes to trimerisation and provides a certain distance to the cell membrane, but is not part of the OX40 binding domain) and the first and the second peptide linkers independently have a length of 3-8 amino acids.

In some embodiments, the OX40 agonist is an OX40 agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an OX40 binding domain.

In some embodiments, the OX40 agonist is MEDI6383. MEDI6383 is an OX40 agonistic fusion protein and can be prepared as described in U.S. Pat. No. 6,312,700, the disclosure of which is incorporated by reference herein.

In some embodiments, the OX40 agonist is an OX40 agonistic scFv antibody comprising any of the foregoing V_{H} domains linked to any of the foregoing V_{L} domains.

In some embodiments, the OX40 agonist is Creative Biolabs OX40 agonist monoclonal antibody MOM-18455, commercially available from Creative Biolabs, Inc., Shirley, NY, USA.

In some embodiments, the OX40 agonist is OX40 agonistic antibody clone Ber-ACT35 commercially available from BioLegend, Inc., San Diego, CA, USA.

I. Optional Cell Viability Analyses

Optionally, a cell viability assay can be performed after the priming first expansion (sometimes referred to as the initial bulk expansion), using standard assays known in the art. Thus, in certain embodiments, the method comprises performing a cell viability assay subsequent to the priming first expansion. For example, a trypan blue exclusion assay can be done on a sample of the bulk TILs, which selectively labels dead cells and allows a viability assessment. Other assays for use in testing viability can include but are not limited to the Alamar blue assay; and the MTT assay.

1. Cell Counts, Viability, Flow Cytometry

In some embodiments, cell counts and/or viability are measured. The expression of markers such as but not limited CD3, CD4, CD8, and CD56, as well as any other disclosed or described herein, can be measured by flow cytometry with antibodies, for example but not limited to those commercially available from BD Bio-sciences (BD Biosciences, San Jose, Calif.) using a FACSCanto™ flow cytometer (BD Biosciences). The cells can be counted manually using a disposable c-chip hemocytometer (VWR, Batavia, IL) and viability can be assessed using any method known in the art, including but not limited to trypan blue staining. The cell viability can also be assayed based on U.S. Ser. No. 15/863,634, incorporated by reference herein in its entirety. Cell viability can also be assayed based on U.S. Patent Publication No. 2018/0280436 or International Patent Publication No. WO/2018/081473, both of which are incorporate herein in their entireties for all purposes.

In some cases, the bulk TIL population can be cryopreserved immediately, using the protocols discussed below. Alternatively, the bulk TIL population can be subjected to REP and then cryopreserved as discussed below. Similarly, in the case where genetically modified TILs will be used in therapy, the bulk or REP TIL populations can be subjected to genetic modifications for suitable treatments.

2. Cell Cultures

In some embodiments, a method for expanding TILs, including those discussed above as well as exemplified in FIG. 1, in particular, e.g., FIG. 1B and/or FIG. 1C and/or FIG. 1D and/or FIG. 1E and/or FIG. 1F and/or FIG. 1G and/or FIG. 1H, may include using about 5,000 mL to about 25,000 mL of cell medium, about 5,000 mL to about 10,000 mL of cell medium, or about 5,800 mL to about 8,700 mL of cell medium. In some embodiments, the media is a serum free medium. In some embodiments, the media in the priming first expansion is serum free. In some embodiments, the media in the second expansion is serum free. In some embodiments, the media in the priming first expansion and the second expansion (also referred to as rapid second expansion) are both serum free. In some embodiments, expanding the number of TILs uses no more than one type of cell culture medium. Any suitable cell culture medium may be used; e.g., AIM-V cell medium (L-glutamine, 50 μM streptomycin sulfate, and 10 μM gentamicin sulfate) cell culture medium (Invitrogen, Carlsbad Calif.). In this regard, the inventive methods advantageously reduce the amount of medium and the number of types of medium required to expand the number of TIL. In some embodiments, expanding the number of TIL may comprise feeding the cells no more frequently than every third or fourth day. Expanding the number of cells in a gas permeable container simplifies the procedures necessary to expand the number of cells by reducing the feeding frequency necessary to expand the cells.

In some embodiments, the cell culture medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME).

In some embodiments, the duration of the method comprising obtaining a tumor tissue sample from the mammal; culturing the tumor tissue sample in a first gas permeable container containing cell medium including IL-2, 1X antigen-presenting feeder cells, and OKT-3 for a duration of about 1 to 8 days, e.g., about 8 days as a priming first expansion; transferring the TILs to a second gas permeable container and expanding the number of TILs in the second gas permeable container containing cell medium including IL-2, 2X antigen-presenting feeder cells, and OKT-3 for a duration of about 7 to 9 days, e.g., about 7 days, about 8 days, or about 9 days.

In some embodiments, the duration of the method comprising obtaining a tumor tissue sample from the mammal; culturing the tumor tissue sample in a first gas permeable container containing cell medium including IL-2, 1X antigen-presenting feeder cells, and OKT-3 for a duration of about 1 to 7 days, e.g., about 7 days as a priming first expansion; transferring the TILs to a second gas permeable container and expanding the number of TILs in the second gas permeable container containing cell medium including IL-2, 2X antigen-presenting feeder cells, and OKT-3 for a duration of about 7 to 9 days, e.g., about 7 days, about 8 days, or about 9 days.

In some embodiments, the duration of the method comprising obtaining a tumor tissue sample from the mammal; culturing the tumor tissue sample in a first gas permeable container containing cell medium including IL-2, 1x antigen-presenting feeder cells, and OKT-3 for a duration of about 1 to 7 days, e.g., about 7 days as a priming first expansion; transferring the TILs to a second gas permeable container and expanding the number of TILs in the second gas permeable container containing cell medium including IL-2, 2X antigen-presenting feeder cells, and OKT-3 for a duration of about 7 to 10 days, e.g., about 7 days, about 8 days, about 9 days or about 10 days.

In some embodiments, TILs are expanded in gas-permeable containers. Gas-permeable containers have been used to expand TILs using PBMCs using methods, compositions, and devices known in the art, including those described in U.S. Patent Application Publication No. 2005/0106717 A1, the disclosures of which are incorporated herein by reference. In some embodiments, TILs are expanded in gas-permeable bags. In some embodiments, TILs are expanded using a cell expansion system that expands TILs in gas permeable bags, such as the Xuri Cell Expansion System W25 (GE Healthcare). In some embodiments, TILs are expanded using a cell expansion system that expands TILs in gas permeable bags, such as the WAVE Bioreactor System, also known as the Xuri Cell Expansion System W5 (GE Healthcare). In some embodiments, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L.

In some embodiments, TILs can be expanded in G-Rex flasks (commercially available from Wilson Wolf Manufacturing). Such embodiments allow for cell populations to expand from about 5×10⁵ cells/cm² to between 10×10⁶ and 30×10⁶ cells/cm². In some embodiments this is without feeding. In some embodiments, this is without feeding so long as medium resides at a height of about 10 cm in the G-Rex flask. In some embodiments this is without feeding but with the addition of one or more cytokines. In some embodiments, the cytokine can be added as a bolus without any need to mix the cytokine with the medium. Such containers, devices, and methods are known in the art and have been used to expand TILs, and include those described in U.S. Patent Application Publication No. US 2014/0377739A1, International Publication No. WO 2014/210036 A1, U.S. Patent Application Publication No. us 2013/0115617 A1, International Publication No. WO 2013/188427 A1, U.S. Patent Application Publication No. US 2011/0136228 A1, U.S. Pat. No. 8,809,050 B2, International publication No. WO 2011/072088 A2, U.S. Patent Application Publication No. US 2016/0208216 A1, U.S. Patent Application Publication No. US 2012/0244133 A1, International Publication No. WO 2012/129201 A1, U.S. Patent Application Publication No. US 2013/0102075 A1, U.S. Pat. No. 8,956,860 B2, International Publication No. WO 2013/173835 A1, U.S. Patent Application Publication No. US 2015/0175966 A1, the disclosures of which are incorporated herein by reference. Such processes are also described in Jin et al., J. Immunotherapy, 2012, 35:283-292.

J. Optional Genetic Engineering of TILs

In some embodiments, the expanded TILs of the present invention are further manipulated before, during, or after an expansion step, including during closed, sterile manufacturing processes, each as provided herein, in order to alter protein expression in a transient manner. In some embodiments, the transiently altered protein expression is due to transient gene editing. In some embodiments, the expanded TILs of the present invention are treated with transcription factors (TFs) and/or other molecules capable of transiently altering protein expression in the TILs. In some embodiments, the TFs and/or other molecules that are capable of transiently altering protein expression provide for altered expression of tumor antigens and/or an alteration in the number of tumor antigen-specific T cells in a population of TILs.

In certain embodiments, the method comprises genetically editing a population of TILs. In certain embodiments, the method comprises genetically editing the first population of TILs, the second population of TILs and/or the third population of TILs.

In some embodiments, the present invention includes genetic editing through nucleotide insertion, such as through ribonucleic acid (RNA) insertion, including insertion of messenger RNA (mRNA) or small (or short) interfering RNA (siRNA), into a population of TILs for promotion of the expression of one or more proteins or inhibition of the expression of one or more proteins, as well as simultaneous combinations of both promotion of one set of proteins with inhibition of another set of proteins.

In some embodiments, the expanded TILs of the present invention undergo transient alteration of protein expression. In some embodiments, the transient alteration of protein expression occurs in the bulk TIL population prior to first expansion, including, for example in the TIL population obtained from for example, Step A as indicated in FIG. 1 (particularly FIG. 1B and FIG. 1C). In some embodiments, the transient alteration of protein expression occurs during the first expansion, including, for example in the TIL population expanded in for example, Step B as indicated in FIG. 1 (for example FIG. 1B). In some embodiments, the transient alteration of protein expression occurs after the first expansion, including, for example in the TIL population in transition between the first and second expansion (e.g. the second population of TILs as described herein), the TIL population obtained from for example, Step B and included in Step C as indicated in FIG. 1. In some embodiments, the transient alteration of protein expression occurs in the bulk TIL population prior to second expansion, including, for example in the TIL population obtained from for example, Step C and prior to its expansion in Step D as indicated in FIG. 1. In some embodiments, the transient alteration of protein expression occurs during the second expansion, including, for example in the TIL population expanded in for example, Step D as indicated in FIG. 1 (e.g. the third population of TILs). In some embodiments, the transient alteration of protein expression occurs after the second expansion, including, for example in the TIL population obtained from the expansion in for example, Step D as indicated in FIG. 1.

In some embodiments, a method of transiently altering protein expression in a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. In some embodiments, a method of transiently altering protein expression in population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of transiently altering protein expression in a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of transiently altering protein expression in a population of TILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments, the TILs of the present invention are further modified to transiently or permanently suppress the expression of one or more genes using the methods described in International Patent Application Nos. WO 2019/136456 A1 or WO 2019/210131 A1, the disclosures of each of which are incorporated by reference herein, including methods described therein to genetically edit TILs to knockout specific target genes such as the genes that code for PD-1 and CTLA-4.

In some embodiments, transient alteration of protein expression results in an increase in Stem Memory T cells (TSCMs). TSCMs are early progenitors of antigen-experienced central memory T cells. TSCMs generally display the long-term survival, self-renewal, and multipotency abilities that define stem cells, and are generally desirable for the generation of effective TIL products. TSCM have shown enhanced anti-tumor activity compared with other T cell subsets in mouse models of adoptive cell transfer (Gattinoni et al. Nat Med 2009, 2011; Gattinoni, Nature Rev. Cancer, 2012; Cieri et al. Blood 2013). In some embodiments, transient alteration of protein expression results in a TIL population with a composition comprising a high proportion of TSCM. In some embodiments, transient alteration of protein expression results in an at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% increase in TSCM percentage. In some embodiments, transient alteration of protein expression results in an at least a 1-fold; 2-fold, 3-fold, 4-fold, 5-fold, or 10-fold increase in TSCMs in the TIL population. In some embodiments, transient alteration of protein expression results in a TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs. In some embodiments, transient alteration of protein expression results in a therapeutic TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs.

In some embodiments, transient alteration of protein expression results in rejuvenation of antigen-experienced T-cells. In some embodiments, rejuvenation includes, for example, increased proliferation, increased T-cell activation, and/or increased antigen recognition.

In some embodiments, transient alteration of protein expression alters the expression in a large fraction of the T-cells in order to preserve the tumor-derived TCR repertoire. In some embodiments, transient alteration of protein expression does not alter the tumor-derived TCR repertoire. In some embodiments, transient alteration of protein expression maintains the tumor-derived TCR repertoire.

In some embodiments, transient alteration of protein results in altered expression of a particular gene. In some embodiments, the transient alteration of protein expression targets a gene including but not limited to PD-1 (also referred to as PDCD1 or CC279), TGFBR2, CCR4/5, CBLB (CBL-B), CISH, CCRs (chimeric co-stimulatory receptors), IL-2, IL-12, IL-15, IL-21, NOTCH 1/2 ICD, TIM3, LAG3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, thymocyte selection associated high mobility group (HMG) box (TOX), ankyrin repeat domain 11 (ANKRD11), BCL6 co-repressor (BCOR) nkyrin repeat domain 11 (ANKRD11), BCL6 co-repressor (BCOR) and/or cAMP protein kinase A (PKA). In some embodiments, the transient alteration of protein expression targets a gene selected from the group consisting of PD-1, TGFBR2, CCR4/5, CBLB (CBL-B), CISH, CCRs (chimeric co-stimulatory receptors), IL-2, IL-12, IL-15, IL-21, NOTCH 1/2 ICD, TIM3, LAG3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, thymocyte selection associated high mobility group (HMG) box (TOX), ankyrin repeat domain 11 (ANKRD11), BCL6 co-repressor (BCOR) nkyrin repeat domain 11 (ANKRD11), BCL6 co-repressor (BCOR) and/or cAMP protein kinase A (PKA). In some embodiments, the transient alteration of protein expression targets PD-1. In some embodiments, the transient alteration of protein expression targets TGFBR2. In some embodiments, the transient alteration of protein expression targets CCR4/5. In some embodiments, the transient alteration of protein expression targets CBLB. In some embodiments, the transient alteration of protein expression targets CISH. In some embodiments, the transient alteration of protein expression targets CCRs (chimeric co-stimulatory receptors). In some embodiments, the transient alteration of protein expression targets IL-2. In some embodiments, the transient alteration of protein expression targets IL-12. In some embodiments, the transient alteration of protein expression targets IL-15. In some embodiments, the transient alteration of protein expression targets IL-21. In some embodiments, the transient alteration of protein expression targets NOTCH 1/2 ICD. In some embodiments, the transient alteration of protein expression targets TIM3. In some embodiments, the transient alteration of protein expression targets LAG3. In some embodiments, the transient alteration of protein expression targets TIGIT. In some embodiments, the transient alteration of protein expression targets TGFβ. In some embodiments, the transient alteration of protein expression targets CCR1. In some embodiments, the transient alteration of protein expression targets CCR2. In some embodiments, the transient alteration of protein expression targets CCR4. In some embodiments, the transient alteration of protein expression targets CCR5. In some embodiments, the transient alteration of protein expression targets CXCR1. In some embodiments, the transient alteration of protein expression targets CXCR2. In some embodiments, the transient alteration of protein expression targets CSCR3. In some embodiments, the transient alteration of protein expression targets CCL2 (MCP-1). In some embodiments, the transient alteration of protein expression targets CCL3 (MIP-1α). In some embodiments, the transient alteration of protein expression targets CCL4 (MIP1-β). In some embodiments, the transient alteration of protein expression targets CCL5 (RANTES). In some embodiments, the transient alteration of protein expression targets CXCL1. In some embodiments, the transient alteration of protein expression targets CXCL8. In some embodiments, the transient alteration of protein expression targets CCL22. In some embodiments, the transient alteration of protein expression targets CCL17. In some embodiments, the transient alteration of protein expression targets VHL. In some embodiments, the transient alteration of protein expression targets CD44. In some embodiments, the transient alteration of protein expression targets PIK3CD. In some embodiments, the transient alteration of protein expression targets SOCS1. In some embodiments, the transient alteration of protein expression targets thymocyte selection associated high mobility group (HMG) box (TOX). In some embodiments, the transient alteration of protein expression targets ankyrin repeat domain 11 (ANKRD11). In some embodiments, the transient alteration of protein expression targets BCL6 co-repressor (BCOR). In some embodiments, the transient alteration of protein expression targets cAMP protein kinase A (PKA).

In some embodiments, the transient alteration of protein expression results in increased and/or overexpression of a chemokine receptor. In some embodiments, the chemokine receptor that is overexpressed by transient protein expression includes a receptor with a ligand that includes but is not limited to CCL2 (MCP-1), CCL3 (MIP-1a), CCL4 (MIP1-β), CCL5 (RANTES), CXCL1, CXCL8, CCL22, and/or CCL17.

In some embodiments, the transient alteration of protein expression results in a decrease and/or reduced expression of PD-1, CTLA-4, TIM-3, LAG-3, TIGIT, TGFI3R2, and/or TGFI3 (including resulting in, for example, TGFI3 pathway blockade). In some embodiments, the transient alteration of protein expression results in a decrease and/or reduced expression of CBLB (CBL-B). In some embodiments, the transient alteration of protein expression results in a decrease and/or reduced expression of CISH.

In some embodiments, the transient alteration of protein expression results in increased and/or overexpression of chemokine receptors in order to, for example, improve TIL trafficking or movement to the tumor site. In some embodiments, the transient alteration of protein expression results in increased and/or overexpression of a CCR (chimeric co-stimulatory receptor). In some embodiments, the transient alteration of protein expression results in increased and/or overexpression of a chemokine receptor selected from the group consisting of CCR1, CCR2, CCR4, CCR5, CXCR1, CXCR2, and/or CSCR3.

In some embodiments, the transient alteration of protein expression results in increased and/or overexpression of an interleukin. In some embodiments, the transient alteration of protein expression results in increased and/or overexpression of an interleukin selected from the group consisting of IL-2, IL-12, IL-15, and/or IL-21.

In some embodiments, the transient alteration of protein expression results in increased and/or overexpression of NOTCH 1/2 ICD. In some embodiments, the transient alteration of protein expression results in increased and/or overexpression of VHL. In some embodiments, the transient alteration of protein expression results in increased and/or overexpression of CD44. In some embodiments, the transient alteration of protein expression results in increased and/or overexpression of PIK3CD. In some embodiments, the transient alteration of protein expression results in increased and/or overexpression of SOCS1,

In some embodiments, the transient alteration of protein expression results in decreased and/or reduced expression of cAMP protein kinase A (PKA).

In some embodiments, the transient alteration of protein expression results in decreased and/or reduced expression of a molecule selected from the group consisting of PD-1, LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of two molecules selected from the group consisting of PD-1, LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of PD-1 and one molecule selected from the group consisting of LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of PD-1, LAG-3, CISH, CBLB, TIM3, and combinations thereof. In some embodiments, the transient alteration of protein expression results in decreased and/or reduced expression of PD-1 and one of LAG3, CISH, CBLB, TIM3, and combinations thereof. In some embodiments, the transient alteration of protein expression results in decreased and/or reduced expression of PD-1 and LAG3. In some embodiments, the transient alteration of protein expression results in decreased and/or reduced expression of PD-1 and CISH. In some embodiments, the transient alteration of protein expression results in decreased and/or reduced expression of PD-1 and CBLB. In some embodiments, the transient alteration of protein expression results in decreased and/or reduced expression of LAG3 and CISH. In some embodiments, the transient alteration of protein expression results in decreased and/or reduced expression of LAG3 and CBLB. In some embodiments, the transient alteration of protein expression results in decreased and/or reduced expression of CISH and CBLB. In some embodiments, the transient alteration of protein expression results in decreased and/or reduced expression of TIM3 and PD-1. In some embodiments, the transient alteration of protein expression results in decreased and/or reduced expression of TIM3 and LAG3. In some embodiments, the transient alteration of protein expression results in decreased and/or reduced expression of TIM3 and CISH. In some embodiments, the transient alteration of protein expression results in decreased and/or reduced expression of TIM3 and CBLB.

In some embodiments, an adhesion molecule selected from the group consisting of CCR2, CCR4, CCR5, CXCR2, CXCR3, CX3CR1, and combinations thereof, is inserted by a gammaretroviral or lentiviral method into the first population of TILs, second population of TILs, or harvested population of TILs (e.g., the expression of the adhesion molecule is increased).

In some embodiments, the transient alteration of protein expression results in decreased and/or reduced expression of a molecule selected from the group consisting of PD-1, LAG3, TIM3, CTLA-4, TIGIT, CISH, TGFβR2, PKA, CBLB, BAFF (BR3), and combinations thereof, and increased and/or enhanced expression of CCR2, CCR4, CCR5, CXCR2, CXCR3, CX3CR1, and combinations thereof. In some embodiments, the transient alteration of protein expression results in decreased and/or reduced expression of a molecule selected from the group consisting of PD-1, LAG3, TIM3, CISH, CBLB, and combinations thereof, and increased and/or enhanced expression of CCR2, CCR4, CCR5, CXCR2, CXCR3, CX3CR1, and combinations thereof

In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

In some embodiments, there is an increase in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is an increase in expression of at least about 65%, about 70%, about 75%, about 80%, about 85%, about 90%, or about 95%. In some embodiments, there is an increase in expression of at least about 75%, about 80%, about 85%, about 90%, or about 95%. In some embodiments, there is an increase in expression of at least about 80%, about 85%, about 90%, or about 95%. In some embodiments, there is an increase in expression of at least about 85%, about 90%, or about 95%. In some embodiments, there is an increase in expression of at least about 80%. In some embodiments, there is an increase in expression of at least about 85%, In some embodiments, there is an increase in expression of at least about 90%. In some embodiments, there is an increase in expression of at least about 95%. In some embodiments, there is an increase in expression of at least about 99%.

In some embodiments, transient alteration of protein expression is induced by treatment of the TILs with transcription factors (TFs) and/or other molecules capable of transiently altering protein expression in the TILs. In some embodiments, the SQZ vector-free microfluidic platform is employed for intracellular delivery of the transcription factors (TFs) and/or other molecules capable of transiently altering protein expression. Such methods demonstrating the ability to deliver proteins, including transcription factors, to a variety of primary human cells, including T cells, have been described in U.S. Patent Application Publication Nos. US 2019/0093073 A1, US 2018/0201889 A1, and US 2019/0017072 A1, the disclosures of each of which are incorporated by reference herein. Such methods can be employed with the present invention in order to expose a population of TILs to transcription factors (TFs) and/or other molecules capable of inducing transient protein expression, wherein said TFs and/or other molecules capable of inducing transient protein expression provide for increased expression of tumor antigens and/or an increase in the number of tumor antigen-specific T cells in the population of TILs, thus resulting in reprogramming of the TIL population and an increase in therapeutic efficacy of the reprogrammed TIL population as compared to a non-reprogrammed TIL population. In some embodiments, the reprogramming results in an increased subpopulation of effector T cells and/or central memory T cells relative to the starting or prior population (i.e., prior to reprogramming) population of TILs, as described herein.

In some embodiments, the transcription factor (TF) includes but is not limited to TCF-1, NOTCH 1/2 ICD, and/or MYB. In some embodiments, the transcription factor (TF) is TCF-1. In some embodiments, the transcription factor (TF) is NOTCH 1/2 ICD. In some embodiments, the transcription factor (TF) is MYB. In some embodiments, the transcription factor (TF) is administered with induced pluripotent stem cell culture (iPSC), such as the commercially available KNOCKOUT Serum Replacement (Gibco/ThermoFisher), to induce additional TIL reprogramming. In some embodiments, the transcription factor (TF) is administered with an iPSC cocktail to induce additional TIL reprogramming. In some embodiments, the transcription factor (TF) is administered without an iPSC cocktail. In some embodiments, reprogramming results in an increase in the percentage of TSCMs. In some embodiments, reprogramming results in an increase in the percentage of TSCMs by about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95% TSCMs.

In some embodiments, a method of transient altering protein expression, as described above, may be combined with a method of genetically modifying a population of TILs includes the step of stable incorporation of genes for production of one or more proteins. In certain embodiments, the method comprises a step of genetically modifying a population of TILs. In certain embodiments, the method comprises genetically modifying the first population of TILs, the second population of TILs and/or the third population of TILs. In some embodiments, a method of genetically modifying a population of TILs includes the step of retroviral transduction. In some embodiments, a method of genetically modifying a population of TILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In some embodiments, a method of genetically modifying a population of TILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In some embodiments, a method of genetically modifying a population of TILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, transient alteration of protein expression in TILs is induced by small interfering RNA (siRNA), sometimes known as short interfering RNA or silencing RNA, which is a double stranded RNA molecule, generally 19-25 base pairs in length. siRNA is used in RNA interference (RNAi), where it interferes with expression of specific genes with complementary nucleotide sequences. siRNA may be used to transiently knockdown genes in TILs also modified to CCRs according to the present invention.

In some embodiments, transient alteration of protein expression is a reduction in expression induced by self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. Small interfering RNA (siRNA), sometimes known as short interfering RNA or silencing RNA, is a double stranded RNA molecule, generally 19-25 base pairs in length. siRNA is used in RNA interference (RNAi), where it interferes with expression of specific genes with complementary nucleotide sequences. sdRNA are covalently and hydrophobically modified RNAi compounds that do not require a delivery vehicle to enter cells. sdRNAs are generally asymmetric chemically modified nucleic acid molecules with minimal double stranded regions. sdRNA molecules typically contain single stranded regions and double stranded regions, and can contain a variety of chemical modifications within both the single stranded and double stranded regions of the molecule. Additionally, the sdRNA molecules can be attached to a hydrophobic conjugate such as a conventional and advanced sterol-type molecule, as described herein. sdRNAs and associated methods for making such sdRNAs have also been described extensively in, for example, U.S. Patent Application Publication Nos. US 2016/0304873 A1, US 2019/0211337 A1, US 2009/0131360 A1, and US 2019/0048341 A1, and U.S. Pat. Nos. 10,633,654 and 10,913,948B2, the disclosures of each of which are incorporated by reference herein. To optimize sdRNA structure, chemistry, targeting position, sequence preferences, and the like, an algorithm has been developed and utilized for sdRNA potency prediction. Based on these analyses, functional sdRNA sequences have been generally defined as having over 70% reduction in expression at 1 μM concentration, with a probability over 40%.

Double stranded DNA (dsRNA) can be generally used to define any molecule comprising a pair of complementary strands of RNA, generally a sense (passenger) and antisense (guide) strands, and may include single-stranded overhang regions. The term dsRNA, contrasted with siRNA, generally refers to a precursor molecule that includes the sequence of an siRNA molecule which is released from the larger dsRNA molecule by the action of cleavage enzyme systems, including Dicer.

In some embodiments, the method comprises transient alteration of protein expression in a population of TILs, comprising the use of siRNA or sdRNA. Methods of using sdRNA have been described in Khvorova and Watts, Nat. Biotechnol. 2017, 35, 238-248; Byrne, et al., J. Ocul. Pharmacol. Ther. 2013, 29, 855-864; and Ligtenberg, et al., Mol. Therapy, 2018, in press, the disclosures of which are incorporated by reference herein. In an embodiment, delivery of siRNA is accomplished using electroporation or cell membrane disruption (such as the squeeze or SQZ method). In some embodiments, delivery of sdRNA to a TIL population is accomplished without use of electroporation, SQZ, or other methods, instead using a 1 to 3 day period in which a TIL population is exposed to sdRNA at a concentration of 1 μM/10,000 TILs in medium. In certain embodiments, the method comprises delivery of siRNA or sdRNA to a TILs population comprising exposing the TILs population to siRNA or sdRNA at a concentration of 1 μM/10,000 TILs in medium for a period of between 1 to 3 days. In some embodiments, delivery of siRNA or sdRNA to a TIL population is accomplished using a 1 to 3 day period in which a TIL population is exposed to siRNA or sdRNA at a concentration of 10 μM/10,000 TILs in medium. In some embodiments, delivery of siRNA or sdRNA to a TIL population is accomplished using a 1 to 3 day period in which a TIL population is exposed to siRNA or sdRNA at a concentration of 50 μM/10,000 TILs in medium. In some embodiments, delivery of siRNA or sdRNA to a TIL population is accomplished using a 1 to 3 day period in which a TIL population is exposed to sdRNA at a concentration of between 0.1 μM/10,000 TILs and 50 μM/10,000 TILs in medium. In some embodiments, delivery of siRNA or sdRNA to a TIL population is accomplished using a 1 to 3 day period in which a TIL population is exposed to siRNA or sdRNA at a concentration of between 0.1 μM/10,000 TILs and 50 μM/10,000 TILs in medium, wherein the exposure to siRNA or sdRNA is performed two, three, four, or five times by addition of fresh siRNA or sdRNA to the media. Other suitable processes are described, for example, in U.S. Patent Application Publication No. US 2011/0039914 A1, US 2013/0131141 A1, and US 2013/0131142 A1, and U.S. Pat. No. 9,080,171, the disclosures of which are incorporated by reference herein.

In some embodiments, siRNA or sdRNA is inserted into a population of TILs during manufacturing. In some embodiments, the siRNA or sdRNA encodes RNA that interferes with NOTCH 1/2 ICD, PD-1, CTLA-4 TIM-3, LAG-3, TIGIT, TGFβ, TGFBR2, cAMP protein kinase A (PKA), BAFF BR3, CISH, and/or CBLB. In some embodiments, the reduction in expression is determined based on a percentage of gene silencing, for example, as assessed by flow cytometry and/or qPCR. In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

The self-deliverable RNAi technology based on the chemical modification of siRNAs or sdRNAs can be employed with the methods of the present invention to successfully deliver the siRNA or sdRNAs to the TILs as described herein. The combination of backbone modifications with asymmetric siRNA or sdRNA structure and a hydrophobic ligand (see, for example, Ligtenberg, et al., Mal. Therapy, 2018 and US20160304873) allow sdRNAs or sd RNAs to penetrate cultured mammalian cells without additional formulations and methods by simple addition to the culture media, capitalizing on the nuclease stability of siRNA or sdRNAs. This stability allows the support of constant levels of RNAi-mediated reduction of target gene activity simply by maintaining the active concentration of siRNA or sdRNA in the media. While not being bound by theory, the backbone stabilization of siRNA or sdRNA provides for extended reduction in gene expression effects which can last for months in non-dividing cells.

In some embodiments, over 95% transfection efficiency of TILs and a reduction in expression of the target by various specific siRNA or sdRNA occurs. In some embodiments, siRNA or sdRNAs containing several unmodified ribose residues were replaced with fully modified sequences to increase potency and/or the longevity of RNAi effect. In some embodiments, a reduction in expression effect is maintained for 12 hours, 24 hours, 36 hours, 48 hours, 5 days, 6 days, 7 days, or 8 days or more. In some embodiments, the reduction in expression effect decreases at 10 days or more post siRNA or sdRNA treatment of the TILs. In some embodiments, more than 70% reduction in expression of the target expression is maintained. In some embodiments, more than 70% reduction in expression of the target expression is maintained TILs. In some embodiments, a reduction in expression in the PD-1/PD-L1 pathway allows for the TILs to exhibit a more potent in vivo effect, which is in some embodiments, due to the avoidance of the suppressive effects of the PD-1/PD-L1 pathway. In some embodiments, a reduction in expression of PD-1 by siRNA or sdRNA results in an increase TIL proliferation.

In some embodiments, the siRNA or sdRNA sequences used in the invention exhibit a 70% reduction in expression of the target gene. In some embodiments, the siRNA or sdRNA sequences used in the invention exhibit a 75% reduction in expression of the target gene. In some embodiments, the siRNA or sdRNA sequences used in the invention exhibit an 80% reduction in expression of the target gene. In some embodiments, the siRNA or sdRNA sequences used in the invention exhibit an 85% reduction in expression of the target gene. In some embodiments, the siRNA or sdRNA sequences used in the invention exhibit a 90% reduction in expression of the target gene. In some embodiments, the siRNA or sdRNA sequences used in the invention exhibit a 95% reduction in expression of the target gene. In some embodiments, the siRNA or sdRNA sequences used in the invention exhibit a 99% reduction in expression of the target gene. In some embodiments, the siRNA or sdRNA sequences used in the invention exhibit a reduction in expression of the target gene when delivered at a concentration of about 0.25 μM to about 4 μM. In some embodiments, the siRNA or sdRNA sequences used in the invention exhibit a reduction in expression of the target gene when delivered at a concentration of about 0.25 μM. In some embodiments, the siRNA or sdRNA sequences used in the invention exhibit a reduction in expression of the target gene when delivered at a concentration of about 0.5 μM. In some embodiments, the siRNA or sdRNA sequences used in the invention exhibit a reduction in expression of the target gene when delivered at a concentration of about 0.75 μM. In some embodiments, the siRNA or sdRNA sequences used in the invention exhibit a reduction in expression of the target gene when delivered at a concentration of about 1.0 μM. In some embodiments, the siRNA or sdRNA sequences used in the invention exhibit a reduction in expression of the target gene when delivered at a concentration of about 1.25 μM. In some embodiments, the siRNA or sdRNA sequences used in the invention exhibit a reduction in expression of the target gene when delivered at a concentration of about 1.5 μM. In some embodiments, the siRNA or sdRNA sequences used in the invention exhibit a reduction in expression of the target gene when delivered at a concentration of about 1.75 μM. In some embodiments, the siRNA or sdRNA sequences used in the invention exhibit a reduction in expression of the target gene when delivered at a concentration of about 2.0 μM. In some embodiments, the siRNA or sdRNA sequences used in the invention exhibit a reduction in expression of the target gene when delivered at a concentration of about 2.25 μM. In some embodiments, the siRNA or sdRNA sequences used in the invention exhibit a reduction in expression of the target gene when delivered at a concentration of about 2.5 μM. In some embodiments, the siRNA or sdRNA sequences used in the invention exhibit a reduction in expression of the target gene when delivered at a concentration of about 2.75 μM. In some embodiments, the siRNA or sdRNA sequences used in the invention exhibit a reduction in expression of the target gene when delivered at a concentration of about 3.0 μM. In some embodiments, the siRNA or sdRNA sequences used in the invention exhibit a reduction in expression of the target gene when delivered at a concentration of about 3.25 μM. In some embodiments, the siRNA or sdRNA sequences used in the invention exhibit a reduction in expression of the target gene when delivered at a concentration of about 3.5 μM. In some embodiments, the siRNA or sdRNA sequences used in the invention exhibit a reduction in expression of the target gene when delivered at a concentration of about 3.75 μM. In some embodiments, the siRNA or sdRNA sequences used in the invention exhibit a reduction in expression of the target gene when delivered at a concentration of about 4.0 μM.

In some embodiments, the siRNA or sdRNA oligonucleotide agents comprise one or more modification to increase stability and/or effectiveness of the therapeutic agent, and to effect efficient delivery of the oligonucleotide to the cells or tissue to be treated. Such modifications can include a 2′-O-methyl modification, a 2′-O-Fluro modification, a diphosphorothioate modification, 2′ F modified nucleotide, a2′-O-methyl modified and/or a 2′deoxy nucleotide. In some embodiments, the oligonucleotide is modified to include one or more hydrophobic modifications including, for example, sterol, cholesterol, vitamin D, naphtyl, isobutyl, benzyl, indol, tryptophane, and/or phenyl. In an additional particular embodiment, chemically modified nucleotides are combination of phosphorothioates, 2′-O-methyl, 2′deoxy, hydrophobic modifications and phosphorothioates. In some embodiments, the sugars can be modified and modified sugars can include but are not limited to D-ribose, 2′-O-alkyl (including 2′-O-methyl and 2′-O-ethyl), i.e., 2′-alkoxy, 2′-amino, 2′-S-alkyl, 2′-halo (including 2′-fluoro), T-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described in Augustyns, et al., Nucl. Acids. Res. 18:4711 (1992), the disclosure of which is incorporated by reference herein.

In some embodiments, the double-stranded siRNA or sdRNA oligonucleotide of the invention is double-stranded over its entire length, i.e., with no overhanging single-stranded sequence at either end of the molecule, i.e., is blunt-ended. In some embodiments, the individual nucleic acid molecules can be of different lengths. In other words, a double-stranded oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). In some embodiments, when a single nucleic acid molecule is used a portion of the molecule at either end can remain single-stranded.

In some embodiments, a double-stranded siRNA or sdRNA oligonucleotide of the invention contains mismatches and/or loops or bulges, but is double-stranded over at least about 70% of the length of the oligonucleotide. In some embodiments, a double-stranded siRNA or sdRNA oligonucleotide of the invention is double-stranded over at least about 80% of the length of the oligonucleotide. In other embodiments, a double-stranded oligonucleotide of the invention is double-stranded over at least about 90%-95% of the length of the oligonucleotide. In some embodiments, a double-stranded siRNA or sdRNA oligonucleotide of the invention is double-stranded over at least about 96%-98% of the length of the oligonucleotide. In some embodiments, the double-stranded siRNA or sdRNA oligonucleotide of the invention contains at least or up to 1, 2, 3, 4, 5, 6, 7, 8, 9, 10, 11, 12, 13, 14, or 15 mismatches.

In some embodiments, the siRNA or sdRNA oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²″), hydrogen phosphonate, or phosphoramidite). “Blocking groups” can also include “end blocking groups” or “exonuclease blocking groups” which protect the 5′ and 3′ termini of the oligonucleotide, including modified nucleotides and non-nucleotide exonuclease resistant structures.

In some embodiments, at least a portion of the contiguous polynucleotides within the siRNA or sdRNA are linked by a substitute linkage, e.g., a phosphorothioate linkage.

In some embodiments, chemical modification can lead to at least a 1.5, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, 500 enhancements in cellular uptake of an siRNA or sdRNA. In some embodiments, at least one of the C or U residues includes a hydrophobic modification. In some embodiments, a plurality of Cs and Us contain a hydrophobic modification. In some embodiments, at least 10%, 15%, 20%, 30%, 40%, 50%, 55%, 60% 65%, 70%, 75%, 80%, 85%, 90% or at least 95% of the Cs and Us can contain a hydrophobic modification. In some embodiments, all of the Cs and Us contain a hydrophobic modification.

In some embodiments, the siRNA or sdRNA molecules exhibit enhanced endosomal release through the incorporation of protonatable amines. In some embodiments, protonatable amines are incorporated in the sense strand (in the part of the molecule which is discarded after RISC loading). In some embodiments, the siRNA or sdRNA compounds of the invention comprise an asymmetric compound comprising a duplex region (required for efficient RISC entry of 10-15 bases long) and single stranded region of 4-12 nucleotides long; with a 13 nucleotide duplex. In some embodiments, a 6 nucleotide single stranded region is employed. In some embodiments, the single stranded region of the siRNA or sdRNA comprises 2-12 phosphorothioate intemucleotide linkages (referred to as phosphorothioate modifications). In some embodiments, 6-8 phosphorothioate intemucleotide linkages are employed. In some embodiments, the siRNA or sdRNA compounds of the invention also include a unique chemical modification pattern, which provides stability and is compatible with RISC entry. The guide strand, for example, may also be modified by any chemical modification which confirms stability without interfering with RISC entry. In some embodiments, the chemical modification pattern in the guide strand includes the majority of C and U nucleotides being 2′ F modified and the 5′ end being phosphorylated.

In some embodiments, at least 30% of the nucleotides in the siRNA or sdRNA are modified. In some embodiments, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the siRNA or sdRNA are modified. In some embodiments, 100% of the nucleotides in the siRNA or sdRNA are modified.

In some embodiments, the siRNA or sdRNA molecules have minimal double stranded regions. In some embodiments the region of the molecule that is double stranded ranges from 8-15 nucleotides long. In some embodiments, the region of the molecule that is double stranded is 8, 9, 10, 11, 12, 13, 14 or 15 nucleotides long. In some embodiments the double stranded region is 13 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. In some embodiments, the single stranded region can be 4, 5, 6, 7, 8, 9, 10, 11 or 12 nucleotides long. In some embodiments, the single stranded region can also be less than 4 or greater than 12 nucleotides long. In certain embodiments, the single stranded region is 6 or 7 nucleotides long.

In some embodiments, the siRNA or sdRNA molecules have increased stability. In some instances, a chemically modified siRNA or sdRNA molecule has a half-life in media that is longer than 1, 2, 3, 4, 5, 6, 7, 8, 9, 10, 11, 12, 13, 14, 15, 16, 17, 18, 19, 20, 21, 22, 23, 24 or more than 24 hours, including any intermediate values. In some embodiments, the sd-rxRNA has a half-life in media that is longer than 12 hours.

In some embodiments, the siRNA or sdRNA is optimized for increased potency and/or reduced toxicity. In some embodiments, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. In some embodiments, reduction in 2′F content of a molecule is predicted to reduce toxicity of the molecule. In some embodiments, the number of phosphorothioate modifications in an RNA molecule can influence the uptake of the molecule into a cell, for example the efficiency of passive uptake of the molecule into a cell. In some embodiments, the sdRNA has no 2′F modification and yet are characterized by equal efficacy in cellular uptake and tissue penetration.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contain 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

The self-deliverable RNAi technology provides a method of directly transfecting cells with the RNAi agent (whether siRNA, sdRNA, or other RNAi agents), without the need for additional formulations or techniques. The ability to transfect hard-to-transfect cell lines, high in vivo activity, and simplicity of use, are characteristics of the compositions and methods that present significant functional advantages over traditional siRNA-based techniques, and as such, the sdRNA methods are employed in several embodiments related to the methods of reduction in expression of the target gene in the TILs of the present invention. The sdRNAi methods allows direct delivery of chemically synthesized compounds to a wide range of primary cells and tissues, both ex-vivo and in vivo. The sdRNAs described in some embodiments of the invention herein are commercially available from Advirna LLC, Worcester, MA, USA.

The siRNA and sdRNA are formed as hydrophobically-modified siRNA-antisense oligonucleotide hybrid structures, and are disclosed, for example in Byrne et al., December 2013, J. Ocular Pharmacology and Therapeutics, 29(10): 855-864, the disclosure of which is incorporated by reference herein.

In some embodiments, the siRNA or sdRNA oligonucleotides can be delivered to the TILs described herein using sterile electroporation. In certain embodiments, the method comprises sterile electroporation of a population of TILs to deliver siRNA or sdRNA oligonucleotides.

In some embodiments, the oligonucleotides can be delivered to the cells in combination with a transmembrane delivery system. In some embodiments, this transmembrane delivery system comprises lipids, viral vectors, and the like. In some embodiments, the oligonucleotide agent is a self-delivery RNAi agent, that does not require any delivery agents. In certain embodiments, the method comprises use of a transmembrane delivery system to deliver siRNA or sdRNA oligonucleotides to a population of TILs.

Oligonucleotides and oligonucleotide compositions are contacted with (e.g., brought into contact with, also referred to herein as administered or delivered to) and taken up by TILs described herein, including through passive uptake by TILs. The siRNA or sdRNA can be added to the TILs as described herein during the first expansion, for example Step B, after the first expansion, for example, during Step C, before or during the second expansion, for example before or during Step D, after Step D and before harvest in Step E, during or after harvest in Step F, before or during final formulation and/or transfer to infusion Bag in Step F, as well as before any optional cryopreservation step in Step F. Moreover, siRNA or sdRNA can be added after thawing from any cryopreservation step in Step F. In some embodiments, one or more siRNA or sdRNAs targeting genes as described herein, including PD-1, LAG-3, TIM-3, CISH, and CBLB, may be added to cell culture media comprising TILs and other agents at concentrations selected from the group consisting of 100 nM to 20 mM, 200 nM to 10 mM, 500 nm to 1 mM, 1 μM to 100 μM, and 1 μM to 100 μM. In some embodiments, one or more siRNA or sdRNAs targeting genes as described herein, including PD-1, LAG-3, TIM-3, CISH, and CBLB, may be added to cell culture media comprising TILs and other agents at amounts selected from the group consisting of 0.1 μM siRNA or sdRNA/10,000 TILs/100 μL media, 0.5 μM siRNA or sdRNA/10,000 TILs/100 μL media, 0.75 μM siRNA or sdRNA/10,000 TILs/100 μL media, 1 μM siRNA or sdRNA/10,000 TILs/100 μL media, 1.25 μM siRNA or sdRNA/10,000 TILs/100 μL media, 1.5 μM siRNA or sdRNA/10,000 TILs/100 μL media, 2 μM siRNA or sdRNA/10,000 TILs/100 μL media, 5 μM siRNA or sdRNA/10,000 TILs/100 μL media, or 10 μM siRNA or sdRNA/10,000 TILs/100 μL media. In some embodiments, one or more sdRNAs targeting genes as described herein, including PD-1, LAG-3, TIM-3, CISH, and CBLB, may be added to TIL cultures during the pre-REP or REP stages twice a day, once a day, every two days, every three days, every four days, every five days, every six days, or every seven days.

Oligonucleotide compositions of the invention, including siRNA or sdRNA, can be contacted with TILs as described herein during the expansion process, for example by dissolving siRNA or sdRNA at high concentrations in cell culture media and allowing sufficient time for passive uptake to occur. In certain embodiments, the method of the present invention comprises contacting a population of TILs with an oligonucleotide composition as described herein. In certain embodiments, the method comprises dissolving an oligonucleotide e.g. siRNA or sdRNA in a cell culture media and contacting the cell culture media with a population of TILs. The TILs may be a first population, a second population and/or a third population as described herein.

In some embodiments, delivery of siRNA or sdRNA oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see, e.g., WO 90/14074; WO 91/16024; WO 91/17424; U.S. Pat. No. 4,897,355; Bergan et a 1993. Nucleic Acids Research. 21:3567).

In some embodiments, more than one siRNA or sdRNA is used to reduce expression of a target gene. In some embodiments, one or more of PD-1, TIM-3, CBLB, LAG3 and/or CISH targeting siRNAs or sdRNAs are used together. In some embodiments, a PD-1 siRNA or sdRNA is used with one or more of TIM-3, CBLB, LAG3 and/or CISH in order to reduce expression of more than one gene target. In some embodiments, a LAG3 siRNA or sdRNA is used in combination with a CISH targeting siRNA or sdRNA to reduce gene expression of both targets. In some embodiments, the siRNAs or sdRNAs targeting one or more of PD-1, TIM-3, CBLB, LAG3 and/or CISH herein are commercially available from Advima LLC, Worcester, MA, USA.

In some embodiments, the siRNA or sdRNA targets a gene selected from the group consisting of PD-1, LAG3, TIM3, CTLA-4, TIGIT, CISH, TGFβR2, PKA, CBLB, BAFF (BR3), and combinations thereof. In some embodiments, the siRNA or sdRNA targets a gene selected from the group consisting of PD-1, LAG3, TIM3, CTLA-4, TIGIT, CISH, TGFβR2, PKA, CBLB, BAFF (BR3), and combinations thereof. In some embodiments, one siRNA or sdRNA targets PD-1 and another siRNA or sdRNA targets a gene selected from the group consisting of LAG3, TIM3, CTLA-4, TIGIT, CISH, TGFβR2, PKA, CBLB, BAFF (BR3), and combinations thereof. In some embodiments, the siRNA or sdRNA targets a gene selected from PD-1, LAG-3, CISH, CBLB, TIM3, and combinations thereof. In some embodiments, the siRNA or sdRNA targets a gene selected from PD-1 and one of LAG3, CISH, CBLB, TIM3, and combinations thereof. In some embodiments, one siRNA or sdRNA targets PD-1 and one siRNA or sdRNA targets LAG3. In some embodiments, one siRNA or sdRNA targets PD-1 and one siRNA or sdRNA targets CISH. In some embodiments, one siRNA or sdRNA targets PD-1 and one siRNA or sdRNA targets CBLB. In some embodiments, one siRNA or sdRNA targets LAG3 and one siRNA or sdRNA targets CISH. In some embodiments, one siRNA or sdRNA targets LAG3 and one siRNA or sdRNA targets CBLB. In some embodiments, one siRNA or sdRNA targets CISH and one siRNA or sdRNA targets CBLB. In some embodiments, one siRNA or sdRNA targets TIM3 and one siRNA or sdRNA targets PD-1. In some embodiments, one siRNA or sdRNA targets TIM3 and one siRNA or sdRNA targets LAG3. In some embodiments, one siRNA or sdRNA targets TIM3 and one siRNA or sdRNA targets CISH. In some embodiments, one siRNA or sdRNA targets TIM3 and one siRNA or sdRNA targets CBLB.

As discussed above, embodiments of the present invention provide tumor infiltrating lymphocytes (TILs) that have been genetically modified via gene-editing to enhance their therapeutic effect. Embodiments of the present invention embrace genetic editing through nucleotide insertion (RNA or DNA) into a population of TILs for both promotion of the expression of one or more proteins and inhibition of the expression of one or more proteins, as well as combinations thereof. Embodiments of the present invention also provide methods for expanding TILs into a therapeutic population, wherein the methods comprise gene-editing the TILs. There are several gene-editing technologies that may be used to genetically modify a population of TILs, which are suitable for use in accordance with the present invention.

In some embodiments, the method comprises a method of genetically modifying a population of TILs which include the step of stable incorporation of genes for production of one or more proteins. In some embodiments, a method of genetically modifying a population of TILs includes the step of retroviral transduction. In some embodiments, a method of genetically modifying a population of TILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In some embodiments, a method of genetically modifying a population of TILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In some embodiments, a method of genetically modifying a population of TILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100×, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, the method comprises a method of genetically modifying a population of TILs e.g. a first population, a second population and/or a third population as described herein. In some embodiments, a method of genetically modifying a population of TILs includes the step of stable incorporation of genes for production or inhibition (e.g., silencing) of one ore more proteins. In some embodiments, a method of genetically modifying a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In some embodiments, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of genetically modifying a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Feigner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein. The TILs may be a first population, a second population and/or a third population of TILs as described herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR/Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with TIL expansion methods of the present invention include CRISPR methods, TALE methods, and ZFN methods, which are described in more detail below. According to an embodiment, a method for expanding TILs into a therapeutic population may be carried out in accordance with any embodiment of the methods described herein (e.g., GEN 3 process) or as described in PCT/US2017/058610, PCT/US2018/012605, or PCT/US2018/012633, wherein the method further comprises gene-editing at least a portion of the TILs by one or more of a CRISPR method, a TALE method or a ZFN method, in order to generate TILs that can provide an enhanced therapeutic effect. According to an embodiment, gene-edited TILs can be evaluated for an improved therapeutic effect by comparing them to non-modified TILs in vitro, e.g., by evaluating in vitro effector function, cytokine profiles, etc. compared to unmodified TILs. In certain embodiments, the method comprises gene editing a population of TILs using CRISPR, TALE and/or ZFN methods.

In some embodiments of the present invention, electroporation is used for delivery of a gene editing system, such as CRISPR, TALEN, and ZFN systems. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

A method for expanding TILs into a therapeutic population may be carried out in accordance with any embodiment of the methods described herein (e.g., process GEN 3) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a CRISPR method (e.g., CRISPR/Cas9 or CRISPR/Cpf1). According to particular embodiments, the use of a CRISPR method during the TIL expansion process causes expression of one or more immune checkpoint genes to be silenced or reduced in at least a portion of the therapeutic population of TILs. Alternatively, the use of a CRISPR method during the TIL expansion process causes expression of one or more immune checkpoint genes to be enhanced in at least a portion of the therapeutic population of TILs.

CRISPR stands for “Clustered Regularly Interspaced Short Palindromic Repeats.” A method of using a CRISPR system for gene editing is also referred to herein as a CRISPR method. There are three types of CRISPR systems which incorporate RNAs and Cas proteins, and which may be used in accordance with the present invention: Types I, II, and III. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR/Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR/Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR/Cas system is directly portable to human cells by co-delivery of plasmids expressing the Cas9 endo-nuclease and the necessary crRNA components. Different variants of Cas proteins may be used to reduce targeting limitations (e.g., orthologs of Cas9, such as Cpf1).

Non-limiting examples of genes that may be silenced or inhibited by permanently gene-editing TILs via a CRISPR method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR.

Non-limiting examples of genes that may be enhanced by permanently gene-editing TILs via a CRISPR method include CCR2, CCR4, CCR5, CXCR2, CXCR3, CX3CR1, IL-2, IL12, IL-15, and IL-21.

Examples of systems, methods, and compositions for altering the expression of a target gene sequence by a CRISPR method, and which may be used in accordance with embodiments of the present invention, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, the disclosures of each of which are incorporated by reference herein. Resources for carrying out CRISPR methods, such as plasmids for expressing CRISPR/Cas9 and CRISPR/Cpf1, are commercially available from companies such as GenScript.

In some embodiments, genetic modifications of populations of TILs, as described herein, may be performed using the CRISPR/Cpf1 system as described in U.S. Pat. No. 9,790,490, the disclosure of which is incorporated by reference herein.

A method for expanding TILs into a therapeutic population may be carried out in accordance with any embodiment of the methods described herein (e.g., process 2A) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a TALE method. According to particular embodiments, the use of a TALE method during the TIL expansion process causes expression of one or more immune checkpoint genes to be silenced or reduced in at least a portion of the therapeutic population of TILs. Alternatively, the use of a TALE method during the TIL expansion process causes expression of one or more immune checkpoint genes to be enhanced in at least a portion of the therapeutic population of TILs.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, Ky., USA), and Life Technologies (Grand Island, N.Y., USA). TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing TILs via a TALE method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing TILs via a TALE method include CCR2, CCR4, CCR5, CXCR2, CXCR3, CX3CR1, IL-2, IL12, IL-15, and IL-21.

Examples of systems, methods, and compositions for altering the expression of a target gene sequence by a TALE method, and which may be used in accordance with embodiments of the present invention, are described in U.S. Pat. No. 8,586,526, which is incorporated by reference herein.

A method for expanding TILs into a therapeutic population may be carried out in accordance with any embodiment of the methods described herein (e.g., process GEN 3) or as described in U.S. Patent Application Publication Nos. US 2020/0299644 A1 and US 2020/0121719 A1 and U.S. Pat. No. 10,925,900, the disclosures of each of which are incorporated by reference herein, wherein the method further comprises gene-editing at least a portion of the TILs by a zinc finger or zinc finger nuclease method. According to particular embodiments, the use of a zinc finger method during the TIL expansion process causes expression of one or more immune checkpoint genes to be silenced or reduced in at least a portion of the therapeutic population of TILs. Alternatively, the use of a zinc finger method during the TIL expansion process causes expression of one or more immune checkpoint genes to be enhanced in at least a portion of the therapeutic population of TILs.

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr®) for zinc-finger construction in partnership with Sigma-Aldrich (St. Louis, Mo., USA).

Non-limiting examples of genes that may be silenced or inhibited by permanently gene-editing TILs via a zinc finger method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1,_ANKRD11, and BCOR.

Non-limiting examples of genes that may be enhanced by permanently gene-editing TILs via a zinc finger method include CCR2, CCR4, CCR5, CXCR2, CXCR3, CX3CR1, IL-2, IL12, IL-15, and IL-21.

Examples of systems, methods, and compositions for altering the expression of a target gene sequence by a zinc finger method, which may be used in accordance with embodiments of the present invention, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present invention, are described in Beane, et al., Mol. Therapy, 2015, 23 1380-1390, the disclosure of which is incorporated by reference herein.

In some embodiments, the TILs are optionally genetically engineered to include additional functionalities, including, but not limited to, a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). In certain embodiments, the method comprises genetically engineering a population of TILs to include a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). Aptly, the population of TILs may be a first population, a second population and/or a third population as described herein.

K. Closed Systems for TIL Manufacturing

The present invention provides for the use of closed systems during the TIL culturing process. Such closed systems allow for preventing and/or reducing microbial contamination, allow for the use of fewer flasks, and allow for cost reductions. In some embodiments, the closed system uses two containers.

Such closed systems are well-known in the art and can be found, for example, at http://www.fda.gov/cber/guidelines.htm and https://www.fda.gov/BiologicsBloodVaccines/GuidanceComplianceRegulatoryInformation/Guidanc es/Blood/ucm076779.htm.

Sterile connecting devices (STCDs) produce sterile welds between two pieces of compatible tubing. This procedure permits sterile connection of a variety of containers and tube diameters. In some embodiments, the closed systems include luer lock and heat sealed systems as described in for example, Example 9. In some embodiments, the closed system is accessed via syringes under sterile conditions in order to maintain the sterility and closed nature of the system. In some embodiments, a closed system as described in Example 9 is employed. In some embodiments, the TILs are formulated into a final product formulation container according to the method described in Example 9, section “Final Formulation and Fill”.

In some embodiments, the closed system uses one container from the time the tumor fragments are obtained until the TILs are ready for administration to the patient or cryopreserving. In some embodiments when two containers are used, the first container is a closed G-container and the population of TILs is centrifuged and transferred to an infusion bag without opening the first closed G-container. In some embodiments, when two containers are used, the infusion bag is a HypoThermosol-containing infusion bag. A closed system or closed TIL cell culture system is characterized in that once the tumor sample and/or tumor fragments have been added, the system is tightly sealed from the outside to form a closed environment free from the invasion of bacteria, fungi, and/or any other microbial contamination.

In some embodiments, the reduction in microbial contamination is between about 5% and about 100%. In some embodiments, the reduction in microbial contamination is between about 5% and about 95%. In some embodiments, the reduction in microbial contamination is between about 5% and about 90%. In some embodiments, the reduction in microbial contamination is between about 10% and about 90%. In some embodiments, the reduction in microbial contamination is between about 15% and about 85%. In some embodiments, the reduction in microbial contamination is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99%, or about 100%.

The closed system allows for TIL growth in the absence and/or with a significant reduction in microbial contamination.

Moreover, pH, carbon dioxide partial pressure and oxygen partial pressure of the TIL cell culture environment each vary as the cells are cultured. Consequently, even though a medium appropriate for cell culture is circulated, the closed environment still needs to be constantly maintained as an optimal environment for TIL proliferation. To this end, it is desirable that the physical factors of pH, carbon dioxide partial pressure and oxygen partial pressure within the culture liquid of the closed environment be monitored by means of a sensor, the signal whereof is used to control a gas exchanger installed at the inlet of the culture environment, and the that gas partial pressure of the closed environment be adjusted in real time according to changes in the culture liquid so as to optimize the cell culture environment. In some embodiments, the present invention provides a closed cell culture system which incorporates at the inlet to the closed environment a gas exchanger equipped with a monitoring device which measures the pH, carbon dioxide partial pressure and oxygen partial pressure of the closed environment, and optimizes the cell culture environment by automatically adjusting gas concentrations based on signals from the monitoring device.

In some embodiments, the pressure within the closed environment is continuously or intermittently controlled. That is, the pressure in the closed environment can be varied by means of a pressure maintenance device for example, thus ensuring that the space is suitable for growth of TILs in a positive pressure state, or promoting exudation of fluid in a negative pressure state and thus promoting cell proliferation. By applying negative pressure intermittently, moreover, it is possible to uniformly and efficiently replace the circulating liquid in the closed environment by means of a temporary shrinkage in the volume of the closed environment.

In some embodiments, optimal culture components for proliferation of the TILs can be substituted or added, and including factors such as IL-2 and/or OKT3, as well as combination, can be added.

L. Optional Cryopreservation of TILs

Either the bulk TIL population (for example the second population of TILs) or the expanded population of TILs (for example the third population of TILs) can be optionally cryopreserved. In some embodiments, cryopreservation occurs on the therapeutic TIL population. In some embodiments, cryopreservation occurs on the TILs harvested after the second expansion. In some embodiments, cryopreservation occurs on the TILs in exemplary Step F of FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments, the TILs are cryopreserved in the infusion bag. In some embodiments, the TILs are cryopreserved prior to placement in an infusion bag. In some embodiments, the TILs are cryopreserved and not placed in an infusion bag. In some embodiments, cryopreservation is performed using a cryopreservation medium. In some embodiments, the cryopreservation media contains dimethylsulfoxide (DMSO). This is generally accomplished by putting the TIL population into a freezing solution, e.g. 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See, Sadeghi, et al., Acta Oncologica 2013, 52, 978-986.

When appropriate, the cells are removed from the freezer and thawed in a 37° C. water bath until approximately 4/5 of the solution is thawed. The cells are generally resuspended in complete media and optionally washed one or more times. In some embodiments, the thawed TILs can be counted and assessed for viability as is known in the art.

In some embodiments, a population of TILs is cryopreserved using CS10 cryopreservation media (CryoStor 10, BioLife Solutions). In some embodiments, a population of TILs is cryopreserved using a cryopreservation media containing dimethylsulfoxide (DMSO). In some embodiments, a population of TILs is cryopreserved using a 1:1 (vol:vol) ratio of CS10 and cell culture media. In some embodiments, a population of TILs is cryopreserved using about a 1:1 (vol:vol) ratio of CS10 and cell culture media, further comprising additional IL-2.

As discussed above, and exemplified in Steps A through E as provided in FIG. 1 (in particular, e.g., FIG. 1B and/or FIG. 1C and/or FIG. 1D and/or FIG. 1E and/or FIG. 1F and/or FIG. 1G and/or FIG. 1H), cryopreservation can occur at numerous points throughout the TIL expansion process. In some embodiments, the expanded population of TILs after the second expansion (as provided for example, according to Step D of FIG. 1 (in particular, e.g., FIG. 1B and/or FIG. 1C and/or FIG. 1D and/or FIG. 1E and/or FIG. 1F and/or FIG. 1G and/or FIG. 1H) can be cryopreserved. Cryopreservation can be generally accomplished by placing the TIL population into a freezing solution, e.g., 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See Sadeghi, et al., Acta Oncologica 2013, 52, 978-986. In some embodiments, the TILs are cryopreserved in 5% DMSO. In some embodiments, the TILs are cryopreserved in cell culture media plus 5% DMSO. In some embodiments, the TILs are cryopreserved according to the methods provided in Example D.

When appropriate, the cells are removed from the freezer and thawed in a 37° C. water bath until approximately 4/5 of the solution is thawed. The cells are generally resuspended in complete media and optionally washed one or more times. In some embodiments, the thawed TILs can be counted and assessed for viability as is known in the art.

In some cases, the Step B TIL population can be cryopreserved immediately, using the protocols discussed below. Alternatively, the bulk TIL population can be subjected to Step C and Step D and then cryopreserved after Step D. Similarly, in the case where genetically modified TILs will be used in therapy, the Step B or Step D TIL populations can be subjected to genetic modifications for suitable treatments.

M. Phenotypic Characteristics of Expanded TILs

In some embodiment, the TILs are analyzed for expression of numerous phenotype markers after expansion, including those described herein and in the Examples. In some embodiments, expression of one or more phenotypic markers is examined. In some embodiments, the phenotypic characteristics of the TILs are analyzed after the first expansion in Step B. In some embodiments, the phenotypic characteristics of the TILs are analyzed during the transition in Step C. In some embodiments, the phenotypic characteristics of the TILs are analyzed during the transition according to Step C and after cryopreservation. In some embodiments, the phenotypic characteristics of the TILs are analyzed after the second expansion according to Step D. In some embodiments, the phenotypic characteristics of the TILs are analyzed after two or more expansions according to Step D.

In some embodiments, the marker is selected from the group consisting of CD8 and CD28. In some embodiments, expression of CD8 is examined. In some embodiments, expression of CD28 is examined. In some embodiments, the expression of CD8 and/or CD28 is higher on TILs produced according the current invention process, as compared to other processes (e.g., the Gen 3 process as provided for example in FIG. 1 (in particular, e.g., FIG. 1B), as compared to the 2A process as provided for example in FIG. 1 (in particular, e.g., FIG. 1B). In some embodiments, the expression of CD8 is higher on TILs produced according the current invention process, as compared to other processes (e.g., the Gen 3 process as provided for example in FIG. 1 (in particular, e.g., FIG. 1B and/or FIG. 1C and/or FIG. 1D and/or FIG. 1E and/or FIG. 1F and/or FIG. 1G and/or FIG. 1H), as compared to the 2A process as provided for example in FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments, the expression of CD28 is higher on TILs produced according the current invention process, as compared to other processes (e.g., the Gen 3 process as provided for example in FIG. 1 (in particular, e.g., FIG. 1B and/or FIG. 1C and/or FIG. 1D and/or FIG. 1E and/or FIG. 1F and/or FIG. 1G and/or FIG. 1H), as compared to the 2A process as provided for example in FIG. 1 (in particular, e.g., FIG. 1A). In some embodiments, high CD28 expression is indicative of a younger, more persistent TIL phenotype. In some embodiments, expression of one or more regulatory markers is measured.

In some embodiments, no selection of the first population of TILs, second population of TILs, third population of TILs, or harvested TIL population based on CD8 and/or CD28 expression is performed during any of the steps for the method for expanding tumor infiltrating lymphocytes (TILs) described herein.

In some embodiments, the percentage of central memory cells is higher on TILs produced according the current invention process, as compared to other processes (e.g., the Gen 3 process as provided for example in FIG. 1 (in particular, e.g., FIG. 1B), as compared to the 2A process as provided for example in FIG. 1 (in particular, e.g., FIG. 1A). In some embodiments the memory marker for central memory cells is selected from the group consisting of CCR7 and CD62L.

In some embodiments, the CD4+ and/or CD8+ TIL Memory subsets can be divided into different memory subsets. In some embodiments, the CD4+ and/or CD8+ TILs comprise the naïve (CD45RA+CD62L+) TILs. In some embodiments, the CD4+ and/or CD8+ TILs comprise the central memory (CM; CD45RA-CD62L+) TILs. In some embodiments, the CD4+ and/or CD8+ TILs comprise the effector memory (EM; CD45RA-CD62L-) TILs. In some embodiments, the CD4+ and/or CD8+ TILs comprise the, RA+ effector memory/effector (TEMRA/TEFF; CD45RA+CD62L+) TILs.

In some embodiments, the TILs express one more markers selected from the group consisting of granzyme B, perforin, and granulysin. In some embodiments, the TILs express granzyme B. In some embodiments, the TILs express perforin. In some embodiments, the TILs express granulysin.

In some embodiments, restimulated TILs can also be evaluated for cytokine release, using cytokine release assays. In some embodiments, TILs can be evaluated for interferon-γ (IFN-γ) secretion. In some embodiments, the IFN-γ secretion is measured by an ELISA assay. In some embodiments, the IFN-γ secretion is measured by an ELISA assay after the rapid second expansion step, after Step D as provided in for example, FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments, TIL health is measured by IFN-gamma (IFN-γ) secretion. In some embodiments, IFN-γ secretion is indicative of active TILs. In some embodiments, a potency assay for IFN-γ production is employed. IFN-γ production is another measure of cytotoxic potential. IFN-γ production can be measured by determining the levels of the cytokine IFN-γ in the media of TIL stimulated with antibodies to CD3, CD28, and CD137/4-1BB. IFN-γ levels in media from these stimulated TIL can be determined using by measuring IFN-γ release. In some embodiments, an increase in IFN-γ production in for example Step D in the Gen 3 process as provided in FIG. 1 (in particular, e.g., FIG. 1B and/or FIG. 1C and/or FIG. 1D and/or FIG. 1E and/or FIG. 1F and/or FIG. 1G and/or FIG. 1H) TILs as compared to for example Step D in the 2A process as provided in FIG. 1 (in particular, e.g., FIG. 1A) is indicative of an increase in cytotoxic potential of the Step D TILs. In some embodiments, IFN-γ secretion is increased one-fold, two-fold, three-fold, four-fold, or five-fold or more. In some embodiments, IFN-γ secretion is increased one-fold. In some embodiments, IFN-γ secretion is increased two-fold. In some embodiments, IFN-γ secretion is increased three-fold. In some embodiments, IFN-γ secretion is increased four-fold. In some embodiments, IFN-γ secretion is increased five-fold. In some embodiments, IFN-γ is measured using a Quantikine ELISA kit. In some embodiments, IFN-γ is measured in TILs ex vivo. In some embodiments, IFN-γ is measured in TILs ex vivo, including TILs produced by the methods of the present invention, including, for example, FIG. 1B and/or FIG. 1C and/or FIG. 1D and/or FIG. 1E and/or FIG. 1F and/or FIG. 1G and/or FIG. 1H methods.

In some embodiments, TILs capable of at least one-fold, two-fold, three-fold, four-fold, or five-fold or more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least one-fold more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least two-fold more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least three-fold more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least four-fold more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least five-fold more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods.

In some embodiments, TILs capable of at least 100 pg/ml to about 1000 pg/mL or more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 200 pg/ml, at least 250 pg/ml, at least 300 pg/ml, at least 350 pg/ml, at least 400 pg/ml, at least 450 pg/ml, at least 500 pg/ml, at least 550 pg/ml, at least 600 pg/ml, at least 650 pg/ml, at least 700 pg/ml, at least 750 pg/ml, at least 800 pg/ml, at least 850 pg/ml, at least 900 pg/ml, at least 950 pg/ml, or at least 1000 pg/mL or more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 200 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 200 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 300 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 400 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 500 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 600 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 700 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 800 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 900 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 1000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 2000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 3000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 4000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 5000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 6000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 7000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 8000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 9000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 10,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 15,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 20,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 25,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 30,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 35,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 40,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 45,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 50,000 pg/ml IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods.

In some embodiments, TILs capable of at least 100 pg/ml/5e5 cells to about 1000 pg/ml/5e5 cells or more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 200 pg/ml/5e5 cells, at least 250 pg/ml/5e5 cells, at least 300 pg/ml/5e5 cells, at least 350 pg/ml/5e5 cells, at least 400 pg/ml/5e5 cells, at least 450 pg/ml/5e5 cells, at least 500 pg/ml/5e5 cells, at least 550 pg/ml/5e5 cells, at least 600 pg/ml/5e5 cells, at least 650 pg/ml/5e5 cells, at least 700 pg/ml/5e5 cells, at least 750 pg/ml/5e5 cells, at least 800 pg/ml/5e5 cells, at least 850 pg/ml/5e5 cells, at least 900 pg/ml/5e5 cells, at least 950 pg/ml/5e5 cells, or at least 1000 pg/ml/5e5 cells or more IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 200 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 200 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 300 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 400 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 500 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 600 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 700 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 800 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 900 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 1000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 2000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 3000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 4000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 5000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 6000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 7000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 8000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 9000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 10,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 15,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 20,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 25,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 30,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 35,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 40,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 45,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 50,000 pg/ml/5e5 cells IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods.

In some embodiments, TILs that exhibit greater than 3000 pg/10⁶ TILs to 300000 pg/10⁶ TILs or more Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILS that exhibit greater than 3000 pg/10⁶ TILs greater than 5000 pg/10⁶ TILs, greater than 7000 pg/10⁶ TILs, greater than 9000 pg/10⁶ TILs, greater than 11000 pg/10⁶ TILs, greater than 13000 pg/10⁶ TILs, greater than 15000 pg/10⁶ TILs, greater than 17000 pg/10⁶ TILs, greater than 19000 pg/10⁶ TILs, greater than 20000 pg/10⁶ TILs, greater than 40000 pg/10⁶ TILs, greater than 60000 pg/10⁶ TILs, greater than 80000 pg/10⁶ TILs, greater than 100000 pg/10⁶ TILs, greater than 120000 pg/10⁶ TILs, greater than 140000 pg/10⁶ TILs, greater than 160000 pg/10⁶ TILs, greater than 180000 pg/10⁶ TILs, greater than 200000 pg/10⁶ TILs, greater than 220000 pg/10⁶ TILs, greater than 240000 pg/10⁶ TILs, greater than 260000 pg/10⁶ TILs, greater than 280000 pg/10⁶ TILs, greater than 300000 pg/10⁶ TILs or more Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 3000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 5000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 7000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 9000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 11000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 13000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 15000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 17000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 19000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 20000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 40000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 60000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 80000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 100000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 120000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 140000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 160000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 180000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 200000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 220000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 240000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 260000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 280000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 300000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 3000 pg/10⁶ TILs to 300000 pg/10⁶ TILs or more Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILS that exhibit greater than 3000 pg/10⁶ TILs greater than 5000 pg/10⁶ TILs, greater than 7000 pg/10⁶ TILs, greater than 9000 pg/10⁶ TILs, greater than 11000 pg/10⁶ TILs, greater than 13000 pg/10⁶ TILs, greater than 15000 pg/10⁶ TILs, greater than 17000 pg/10⁶ TILs, greater than 19000 pg/10⁶ TILs, greater than 20000 pg/10⁶ TILs, greater than 40000 pg/10⁶ TILs, greater than 60000 pg/10⁶ TILs, greater than 80000 pg/10⁶ TILs, greater than 100000 pg/10⁶ TILs, greater than 120000 pg/10⁶ TILs, greater than 140000 pg/10⁶ TILs, greater than 160000 pg/10⁶ TILs, greater than 180000 pg/10⁶ TILs, greater than 200000 pg/10⁶ TILs, greater than 220000 pg/10⁶ TILs, greater than 240000 pg/10⁶ TILs, greater than 260000 pg/10⁶ TILs, greater than 280000 pg/10⁶ TILs, greater than 300000 pg/10⁶ TILs or more Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 3000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 5000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 7000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 9000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 11000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 13000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 15000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 17000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 19000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 20000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 40000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 60000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 80000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 100000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 120000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 140000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 160000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 180000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 200000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 220000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 240000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 260000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 280000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 300000 pg/10⁶ TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H.

In some embodiments, TILs that exhibit greater than 1000 pg/ml to 300000 pg/ml or more Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 1000 pg/ml, greater than 2000 pg/ml, greater than 3000 pg/ml, greater than 4000 pg/ml, greater than 5000 pg/ml, greater than 6000 pg/ml, greater than 7000 pg/ml, greater than 8000 pg/ml greater than 9000 pg/ml greater than 10000 pg/ml, greater than 20000 pg/ml, greater than 30000 pg/ml, greater than 40000 pg/ml, greater than 50000 pg/ml, greater than 60000 pg/ml, greater than 70000 pg/ml, greater than 80000 pg/ml, greater than 90000 pg/ml, greater than 100000 pg/ml or more Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILS that exhibit greater than 1000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 2000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 3000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 4000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 5000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 6000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 7000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 8000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 9000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 10000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILS that exhibit greater than 20000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 30000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 40000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 50000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 60000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 70000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 80000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 90000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 100000 pg/ml Granzyme B are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 120000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 140000 pg/ml Granzyme B are TILs Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 160000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 180000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 200000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 220000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 240000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 260000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 280000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H. In some embodiments, TILs that exhibit greater than 300000 pg/ml Granzyme B secretion are TILs produced by the expansion methods of the present invention, including for example FIG. 1B and/or FIG. 1C and/or FIG. 1E and/or FIG. 1F and/or FIG. 1G and/or FIG. 1H.

In some embodiments, the expansion methods of the present invention produce an expanded population of TILs that exhibits increased Granzyme B secretion in vitro including for example TILs as provided in FIG. 1B and/or FIG. 1C and/or FIG. 1D and/or FIG. 1E and/or FIG. 1F and/or FIG. 1G and/or FIG. 1H, as compared to non-expanded population of TILs. In some embodiments, Granzyme B secretion of the expanded population of TILs of the present invention is increased by at least one-fold to fifty-fold or more as compared to non-expanded population of TILs. In some embodiments, IFN-γ secretion is increased by at least one-fold, at least two-fold, at least three-fold, at least four-fold, at least five-fold, at least six-fold, at least seven-fold, at least eight-fold, at least nine-fold, at least ten-fold, at least twenty-fold, at least thirty-fold, at least forty-fold, at least fifty-fold or more as compared to non-expanded population of TILs. In some embodiments, Granzyme B secretion of the expanded population of TILs of the present invention is increased by at least one-fold as compared to non-expanded population of TILs. In some embodiments, Granzyme B secretion of the expanded population of TILs of the present invention is increased by at least two-fold as compared to non-expanded population of TILs. In some embodiments, Granzyme B secretion of the expanded population of TILs of the present invention is increased by at least three-fold as compared to non-expanded population of TILs. In some embodiments, Granzyme B secretion of the expanded population of TILs of the present invention is increased by at least four-fold as compared to non-expanded population of TILs. In some embodiments, Granzyme B secretion of the expanded population of TILs of the present invention is increased by at least five-fold as compared to non-expanded population of TILs. In some embodiments, Granzyme B secretion of the expanded population of TILs of the present invention is increased by at least six-fold as compared to non-expanded population of TILs. In some embodiments, Granzyme B secretion of the expanded population of TILs of the present invention is increased by at least seven-fold as compared to non-expanded population of TILs. In some embodiments, Granzyme B secretion of the expanded population of TILs of the present invention is increased by at least eight-fold as compared to non-expanded population of TILs. In some embodiments, Granzyme B secretion of the expanded population of TILs of the present invention is increased by at least nine-fold as compared to non-expanded population of TILs. In some embodiments, Granzyme B secretion of the expanded population of TILs of the present invention is increased by at least ten-fold as compared to non-expanded population of TILs. In some embodiments, Granzyme B secretion of the expanded population of TILs of the present invention is increased by at least twenty-fold as compared to non-expanded population of TILs. In some embodiments, Granzyme B secretion of the expanded population of TILs of the present invention is increased by at least thirty-fold as compared to non-expanded population of TILs. In some embodiments, Granzyme B secretion of the expanded population of TILs of the present invention is increased by at least forty-fold as compared to non-expanded population of TILs. In some embodiments, Granzyme B secretion of the expanded population of TILs of the present invention is increased by at least fifty-fold as compared to non-expanded population of TILs.

In some embodiments, TILs capable of at least one-fold, two-fold, three-fold, four-fold, or five-fold or more lower levels of TNF-α (i.e., TNF-alpha) secretion as compared to IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least one-fold lower levels of TNF-α secretion as compared to IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least two-fold lower levels of TNF-α secretion as compared to IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least three-fold lower levels of TNF-α secretion as compared to IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least four-fold lower levels of TNF-α secretion as compared to IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least five-fold lower levels of TNF-α secretion as compared to IFN-γ secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods.

In some embodiments, TILs capable of at least 200 pg/ml/5e5 cells to about 10,000 pg/ml/5e5 cells or more TNF-α (i.e., TNF-alpha) secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 500 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 1000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 2000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 3000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 4000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 5000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments. TILs capable of at least 6000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 7000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 8000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, TILs capable of at least 9000 pg/ml/5e5 cells to about 10,000 pg/ml/5e5 cells or more TNF-α secretion are TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods.

In some embodiments, IFN-γ and granzyme B levels are measured to determine the phenotypic characteristics of the TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, IFN-γ and TNF-α levels are measured to determine the phenotypic characteristics of the TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, granzyme B and TNF-α levels are measured to determine the phenotypic characteristics of the TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods. In some embodiments, IFN-γ, granzyme B and TNF-α levels are measured to determine the phenotypic characteristics of the TILs produced by the expansion methods of the present invention, including, for example FIG. 1B and/or FIG. 1C and/or FIG. 1D and/or FIG. 1E and/or FIG. 1F and/or FIG. 1G and/or FIG. 1H methods.

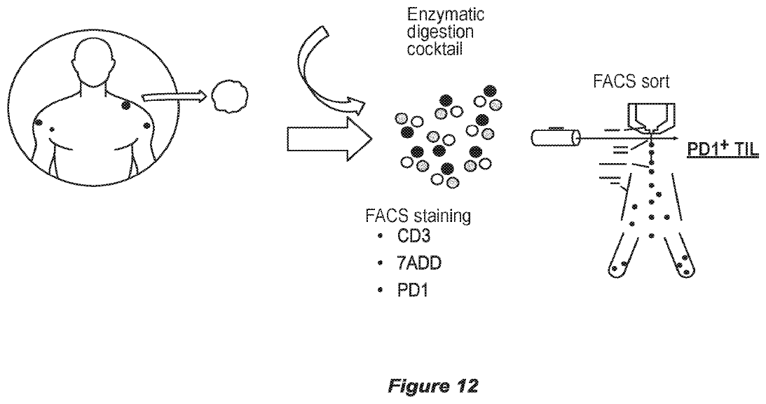

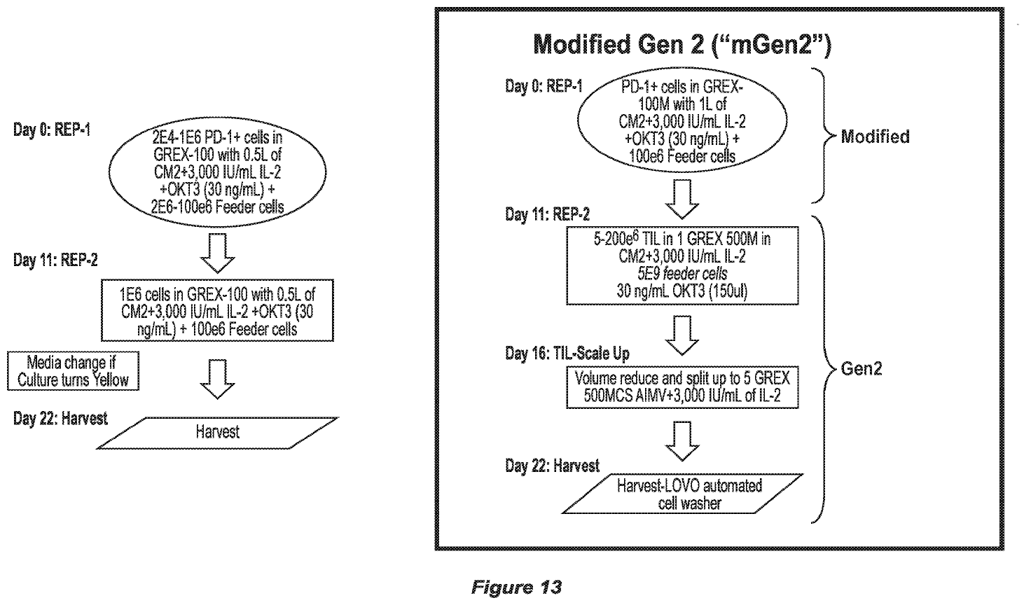

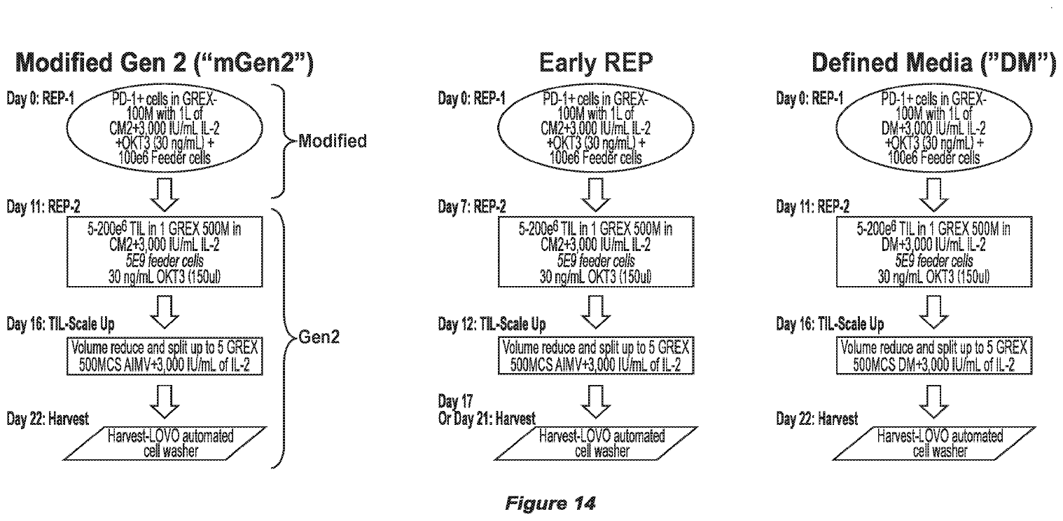

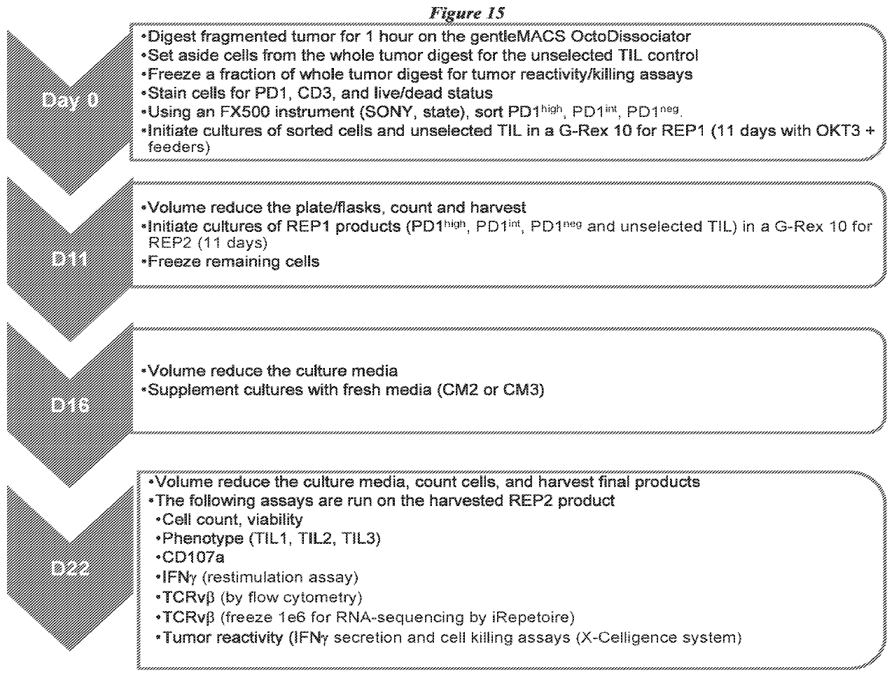

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained by the present method exhibit an increase in the T-cell repertoire diversity as compared to freshly harvested TILs and/or TILs prepared using other methods than those provide herein including, for example, methods other than those embodied in FIG. 1 (in particular, e.g., FIG. 1B and/or FIG. 1C and/or FIG. 1D and/or FIG. 1E and/or FIG. 1F and/or FIG. 1G and/or FIG. 1H). In some embodiments, the TILs obtained by the present method exhibit an increase in the T-cell repertoire diversity as compared to freshly harvested TILs and/or TILs prepared using methods referred to as process 2A, as exemplified in FIG. 1 (in particular, e.g., FIG. 1A). In some embodiments, the TILs obtained in the first expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β). In some embodiments, the process as described herein (e.g., the Gen 3 process) shows higher clonal diversity as compared to other processes, for example the process referred to as the Gen 2 based on the number of unique peptide CDRs within the sample (see, for example FIGS. 12-14).

In some embodiments, the TILs prepared by the methods of the present invention, including those as described for example in FIG. 1, exhibit increased polyclonality as compared to TILs produced by other methods, including those not exemplified in FIG. 1, such as for example, methods referred to as process 1C methods. In some embodiments, significantly improved polyclonality and/or increased polyclonality is indicative of treatment efficacy and/or increased clinical efficacy for cancer treatment. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1. In some embodiments, polyclonality is increased one-fold as compared to an untreated patient and/or as compared to a patient treated with TILs prepared using other methods than those provide herein including for example, methods other than those embodied in FIG. 1. In some embodiments, polyclonality is increased two-fold as compared to an untreated patient and/or as compared to a patient treated with TILs prepared using other methods than those provide herein including for example, methods other than those embodied in FIG. 1. In some embodiments, polyclonality is increased ten-fold as compared to an untreated patient and/or as compared to a patient treated with TILs prepared using other methods than those provide herein including for example, methods other than those embodied in FIG. 1. In some embodiments, polyclonality is increased 100-fold as compared to an untreated patient and/or as compared to a patient treated with TILs prepared using other methods than those provide herein including for example, methods other than those embodied in FIG. 1. In some embodiments, polyclonality is increased 500-fold as compared to an untreated patient and/or as compared to a patient treated with TILs prepared using other methods than those provide herein including for example, methods other than those embodied in FIG. 1. In some embodiments, polyclonality is increased 1000-fold as compared to an untreated patient and/or as compared to a patient treated with TILs prepared using other methods than those provide herein including for example, methods other than those embodied in FIG. 1.

In some embodiments, the activation and exhaustion of TILs can be determined by examining one or more markers. In some embodiments, the activation and exhaustion can be determined using multicolor flow cytometry. In some embodiments, the activation and exhaustion of markers include but not limited to one or more markers selected from the group consisting of CD3, PD-1, 2B4/CD244, CD8, CD25, BTLA, KLRG, TIM-3, CD194/CCR4, CD4, TIGIT, CD183, CD69, CD95, CD127, CD103, and/or LAG-3). In some embodiments, the activation and exhaustion of markers include but not limited to one or more markers selected from the group consisting of BTLA, CTLA-4, ICOS, Ki67, LAG-3, PD-1, TIGIT, and/or TIM-3. In some embodiments, the activation and exhaustion of markers include but not limited to one or more markers selected from the group consisting of BTLA, CTLA-4, ICOS, Ki67, LAG-3, CD103+/CD69+, CD103+/CD69-, PD-1, TIGIT, and/or TIM-3. In some embodiments, the T-cell markers (including activation and exhaustion markers) can be determined and/or analyzed to examine T-cell activation, inhibition, or function. In some embodiments, the T-cell markers can include but are not limited to one or more markers selected from the group consisting of TIGIT, CD3, FoxP3, Tim-3, PD-1, CD103, CTLA-4, LAG-3, BTLA-4, ICOS, Ki67, CD8, CD25, CD45, CD4, and/or CD59.

In some embodiments, the phenotypic characterization is examined after cryopreservation.

N. Additional Process Embodiments

In some embodiments, the invention provides a method for expanding tumor infiltrating lymphocytes (TILs) into a therapeutic population of TILs comprising: (a) obtaining and/or receiving a first population of TILs from a tumor resected from a subject by processing a tumor sample obtained from the subject into multiple tumor fragments; (b) selecting PD-1, CD39, CD38, CD103. CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the PD-1, CD39, CD38, CD103, CD101, LAG3, TIM3 and/or TIGIT enriched TIL population in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs; (d) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and (e) harvesting the therapeutic population of TILs obtained from step (d). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 to 7 days.

In some embodiments, the invention provides a method for expanding tumor infiltrating lymphocytes (TILs) into a therapeutic population of TILs comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the PD-1, CD39, CD38, CD103, CD101, LAG3, TIM3 and/or TIGIT enriched TIL population in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs; (d) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and (e) harvesting the therapeutic population of TILs obtained from step (d). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 4 to 5 days.

In some embodiments, the invention provides a method for expanding tumor infiltrating lymphocytes (TILs) into a therapeutic population of TILs comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the PD-1, CD39, CD38, CD103, CD101, LAG3, TIM3 and/or TIGIT enriched TIL population in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs; (d) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and (e) harvesting the therapeutic population of TILs obtained from step (d). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 days.

In some embodiments, the present provides a method for expanding tumor infiltrating lymphocytes (TILs) into a therapeutic population of TILs comprising: (a) performing a priming first expansion by culturing a first population of TILs which have been selected to be PD-1, CD39, CD38, CD103, CD101, LAG3, TIM3 and/or TIGIT positive, said first population of TILs obtainable by processing a tumor sample from a subject by tumor digestion and selecting for the PD-1, CD39, CD38, CD103, CD101, LAG3, TIM3 and/or TIGIT positive TILs, in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs; (b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area; and (c) harvesting the therapeutic population of TILs obtained from step (b). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 to 7 days.

In some embodiments, the present provides a method for expanding tumor infiltrating lymphocytes (TILs) into a therapeutic population of TILs comprising: (a) performing a priming first expansion by culturing a first population of TILs which have been selected to be PD-1, CD39, CD38, CD103, CD101, LAG3, TIM3 and/or TIGIT positive, said first population of TILs obtainable by processing a tumor sample from a subject by tumor digestion and selecting for the PD-1, CD39, CD38, CD103, CD101, LAG3, TIM3 and/or TIGIT positive TILs, in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs; (b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area; and (c) harvesting the therapeutic population of TILs obtained from step (b). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 4 to 5 days.

In some embodiments, the present provides a method for expanding tumor infiltrating lymphocytes (TILs) into a therapeutic population of TILs comprising: (a) performing a priming first expansion by culturing a first population of TILs which have been selected to be PD-1, CD39, CD38, CD103, CD101, LAG3, TIM3 and/or TIGIT positive, said first population of TILs obtainable by processing a tumor sample from a subject by tumor digestion and selecting for the PD-1, CD39, CD38, CD103, CD101, LAG3, TIM3 and/or TIGIT positive TILs, in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs; (b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area; and (c) harvesting the therapeutic population of TILs obtained from step (b). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 days.

In some embodiments, the present provides a method for expanding tumor infiltrating lymphocytes (TILs) into a therapeutic population of TILs comprising: (a) performing a priming first expansion of a first population of TILs which have been selected to be PD-1, CD39, CD38, CD103, CD101, LAG3, TIM3 and/or TIGIT positive by culturing the first population of TILs in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs; (b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and (c) harvesting the therapeutic population of TILs obtained from step (b). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 to 7 days.

In some embodiments, the present provides a method for expanding tumor infiltrating lymphocytes (TILs) into a therapeutic population of TILs comprising: (a) performing a priming first expansion of a first population of TILs which have been selected to be PD-1, CD39, CD38, CD103, CD101, LAG3, TIM3 and/or TIGIT positive by culturing the first population of TILs in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs; (b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and (c) harvesting the therapeutic population of TILs obtained from step (b). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 1₁; 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 4 to 5 days.

In some embodiments, the present provides a method for expanding tumor infiltrating lymphocytes (TILs) into a therapeutic population of TILs comprising: (a) performing a priming first expansion of a first population of TILs which have been selected to be PD-1, CD39, CD38, CD103, CD101, LAG3, TIM3 and/or TIGIT positive by culturing the first population of TILs in a cell culture medium comprising IL-2; OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs; (b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and (c) harvesting the therapeutic population of TILs obtained from step (b). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 days.

In some embodiments, the present provides a method for treating a subject with cancer, the method comprising administering expanded tumor infiltrating lymphocytes (TILs) comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for about 1 to 7, 8, 9, 10, or 11 days to obtain the second population of TILs; (d) performing a rapid second expansion by supplementing the cell culture medium of the second population of TILs with additional IL-2, OKT-3, and APCs, to produce a third population of TILs, wherein the number of APCs added to the rapid second expansion is at least twice the number of APCs added in step (b), wherein the rapid second expansion is performed for about 1 to 11 days to obtain the third population of TILs, wherein the third population of TILs is a therapeutic population of TILs, wherein the rapid second expansion is performed in a container comprising a second gas-permeable surface area; (e) harvesting the therapeutic population of TILs obtained from step (c); (f) transferring the harvested TIL population from step (d) to an infusion bag; and (g) administering a therapeutically effective dosage of the TILs from step (e) to the subject. In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 to 7 days.

In some embodiments, the present provides a method for treating a subject with cancer, the method comprising administering expanded tumor infiltrating lymphocytes (TILs) comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for about 1 to 7, 8, 9, 10, or 11 days to obtain the second population of TILs; (d) performing a rapid second expansion by supplementing the cell culture medium of the second population of TILs with additional IL-2, OKT-3, and APCs, to produce a third population of TILs, wherein the number of APCs added to the rapid second expansion is at least twice the number of APCs added in step (b), wherein the rapid second expansion is performed for about 1 to 11 days to obtain the third population of TILs, wherein the third population of TILs is a therapeutic population of TILs, wherein the rapid second expansion is performed in a container comprising a second gas-permeable surface area; (e) harvesting the therapeutic population of TILs obtained from step (c); (f) transferring the harvested TIL population from step (d) to an infusion bag; and (g) administering a therapeutically effective dosage of the TILs from step (e) to the subject. In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 4 to 5 days.

In some embodiments, the present provides a method for treating a subject with cancer, the method comprising administering expanded tumor infiltrating lymphocytes (TILs) comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for about 1 to 7, 8, 9, 10, or 11 days to obtain the second population of TILs; (d) performing a rapid second expansion by supplementing the cell culture medium of the second population of TILs with additional IL-2, OKT-3, and APCs, to produce a third population of TILs, wherein the number of APCs added to the rapid second expansion is at least twice the number of APCs added in step (b), wherein the rapid second expansion is performed for about 1 to 11 days to obtain the third population of TILs, wherein the third population of TILs is a therapeutic population of TILs, wherein the rapid second expansion is performed in a container comprising a second gas-permeable surface area; (e) harvesting the therapeutic population of TILs obtained from step (c); (f) transferring the harvested TIL population from step (d) to an infusion bag; and (g) administering a therapeutically effective dosage of the TILs from step (e) to the subject. In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 days.

In some embodiments, the present provides a method for expanding tumor infiltrating lymphocytes (TILs) into a therapeutic population of TILs comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the first population of TILs in a first TIL cell culture comprising a first cell culture medium, IL-2, and either: i) a first culture supernatant obtained from a first culture of antigen-presenting feeder cells (APCs), wherein the first culture supernatant comprises OKT-3, or ii) APCs and OKT-3, wherein the priming first expansion is performed by culturing the first TIL cell culture in a first container comprising a first gas-permeable surface area for a first period of about 1 to 7, 8, 9, 10, or 11 days to obtain a second population of TILs, and wherein the second population of TILs is greater in number than the first population of TILs; (d) performing a rapid second expansion by transferring the first TIL cell culture into a second container comprising a second gas-permeable surface area supplemented with a second cell culture medium, IL-2, and either: i) a second culture supernatant obtained from a second culture of APCs, wherein the second culture supernatant comprises OKT-3, or ii) APCs and OKT-3; to form a second TIL cell culture, wherein the rapid second expansion is performed by culturing the second TIL cell culture for a second period of about 1 to 11 days to obtain a third population of TILs, and wherein the third population of TILs is a therapeutic population of TILs; wherein the first TIL cell culture does not comprise both the first culture supernatant and APCs; wherein the second TIL cell culture does not comprise both the second culture supernatant and supplemental APCs; (e) harvesting the therapeutic population of TILs obtained from step (d); and (f) transferring the harvested TIL population from step (e) to an infusion bag. In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 to 7 days.

In some embodiments, the present provides a method for expanding tumor infiltrating lymphocytes (TILs) into a therapeutic population of TILs comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the first population of TILs in a first TIL cell culture comprising a first cell culture medium, IL-2, and either: i) a first culture supernatant obtained from a first culture of antigen-presenting feeder cells (APCs), wherein the first culture supernatant comprises OKT-3, or ii) APCs and OKT-3, wherein the priming first expansion is performed by culturing the first TIL cell culture in a first container comprising a first gas-permeable surface area for a first period of about 1 to 7, 8, 9, 10, or 11 days to obtain a second population of TILs, and wherein the second population of TILs is greater in number than the first population of TILs; (d) performing a rapid second expansion by transferring the first TIL cell culture into a second container comprising a second gas-permeable surface area supplemented with a second cell culture medium, IL-2, and either: i) a second culture supernatant obtained from a second culture of APCs, wherein the second culture supernatant comprises OKT-3, or ii) APCs and OKT-3; to form a second TIL cell culture, wherein the rapid second expansion is performed by culturing the second TIL cell culture for a second period of about 1 to 11 days to obtain a third population of TILs, and wherein the third population of TILs is a therapeutic population of TILs; wherein the first TIL cell culture does not comprise both the first culture supernatant and APCs; wherein the second TIL cell culture does not comprise both the second culture supernatant and supplemental APCs; (e) harvesting the therapeutic population of TILs obtained from step (d); and (f) transferring the harvested TIL population from step (e) to an infusion bag. In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 4 to 5 days.

In some embodiments, the present provides a method for expanding tumor infiltrating lymphocytes (TILs) into a therapeutic population of TILs comprising: (a) obtaining and/or receiving a first population of TILs from a tumor resected from a subject by processing a tumor sample obtained from the subject into multiple tumor fragments; (b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population; (c) performing a priming first expansion by culturing the first population of TILs in a first TIL cell culture comprising a first cell culture medium, IL-2, and either: i) a first culture supernatant obtained from a first culture of antigen-presenting feeder cells (APCs), wherein the first culture supernatant comprises OKT-3, or ii) APCs and OKT-3, wherein the priming first expansion is performed by culturing the first TIL cell culture in a first container comprising a first gas-permeable surface area for a first period of about 1 to 7, 8, 9, 10, or 11 days to obtain a second population of TILs, and wherein the second population of TILs is greater in number than the first population of TILs; (d) performing a rapid second expansion by transferring the first TIL cell culture into a second container comprising a second gas-permeable surface area supplemented with a second cell culture medium, IL-2, and either: i) a second culture supernatant obtained from a second culture of APCs, wherein the second culture supernatant comprises OKT-3, or ii) APCs and OKT-3; to form a second TIL cell culture, wherein the rapid second expansion is performed by culturing the second TIL cell culture for a second period of about 1 to 11 days to obtain a third population of TILs, and wherein the third population of TILs is a therapeutic population of TILs; wherein the first TIL cell culture does not comprise both the first culture supernatant and APCs; wherein the second TIL cell culture does not comprise both the second culture supernatant and supplemental APCs; (e) harvesting the therapeutic population of TILs obtained from step (d); and (f) transferring the harvested TIL population from step (e) to an infusion bag. In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 days.

In other embodiments, the invention provides the method described in any of the preceding paragraphs as applicable above modified such that in step (b) the primary first expansion is performed by contacting the first population of TILs with a culture medium which further comprises exogenous antigen-presenting cells (APCs), wherein the number of APCs in the culture medium in step (c) is greater than the number of APCs in the culture medium in step (b).

In other embodiments, the invention provides the method described in any of the preceding paragraphs as applicable above modified such that in step (c) the culture medium is supplemented with additional exogenous APCs.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 20:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 10:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 9:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 8:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 7:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 6:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 5:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 4:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 3:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.9:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.8:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.7:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.6:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.5:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.4:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.3:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.2:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.1:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 10:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 5:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 4:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 3:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.9:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.8:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.7:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.6:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.5:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.4:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.3:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.2:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.1:1.

In other embodiments, the invention provides the method described in any of the preceding paragraphs as applicable above modified such that the ratio of number of APCs added in the rapid second expansion to the number of APCs added in step (b) is at or about 2:1.

In other embodiments, the invention provides the method described in any of the preceding paragraphs as applicable above modified such that the ratio of number of APCs added in the rapid second expansion to the number of APCs added in step (b) is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In other embodiments, the invention provides the method described in any of the preceding paragraphs as applicable above modified such that the number of APCs added in the primary first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs, and such that the number of APCs added in the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs.

In other embodiments, the invention provides the method described in any of the preceding paragraphs as applicable above modified such that the number of APCs added in the primary first expansion is selected from the range of at or about 1×10⁸ APCs to at or about 3.5×10⁸ APCs, and wherein the number of APCs added in the rapid second expansion is selected from the range of at or about 3.5×10⁸ APCs to at or about 1×10⁹ APCs.

In other embodiments, the invention provides the method described in any of the preceding paragraphs as applicable above modified such that the number of APCs added in the primary first expansion is selected from the range of at or about 1.5×10⁸ APCs to at or about 3×10⁸ APCs, and wherein the number of APCs added in the rapid second expansion is selected from the range of at or about 4×10⁸ APCs to at or about 7.5×10⁸ APCs.

In other embodiments, the invention provides the method described in any of the preceding paragraphs as applicable above modified such that the number of APCs added in the primary first expansion is selected from the range of at or about 2×10⁸ APCs to at or about 2.5×10⁸ APCs, and wherein the number of APCs added in the rapid second expansion is selected from the range of at or about 4.5×10⁸ APCs to at or about 5.5×10⁸ APCs.

In other embodiments, the invention provides the method described in any of the preceding paragraphs as applicable above modified such that at or about 2.5×10⁸ APCs are added to the primary first expansion and at or about 5×10⁸ APCs are added to the rapid second expansion.

In other embodiments, the invention provides the method described in any of the preceding paragraphs as applicable above modified such that the antigen-presenting cells are peripheral blood mononuclear cells (PBMCs).

In other embodiments, the invention provides the method described in any of the preceding paragraphs as applicable above modified such that the multiple tumor fragments are distributed into a plurality of separate containers, in each of which separate containers the first population of TILs is obtained in step (a), the second population of TILs is obtained in step (b), and the third population of TILs is obtained in step (c), and the therapeutic populations of TILs from the plurality of containers in step (c) are combined to yield the harvested TIL population from step (d).

In other embodiments, the invention provides the method described in any of the preceding paragraphs as applicable above modified such that the multiple tumors are evenly distributed into the plurality of separate containers.

In other embodiments, the invention provides the method described in any of the preceding paragraphs as applicable above modified such that the plurality of separate containers comprises at least two separate containers.

In other embodiments, the invention provides the method described in any of the preceding paragraphs as applicable above modified such that the plurality of separate containers comprises from two to twenty separate containers.

In other embodiments, the invention provides the method described in any of the preceding paragraphs as applicable above modified such that the plurality of separate containers comprises from two to fifteen separate containers.

In other embodiments, the invention provides the method described in any of the preceding paragraphs as applicable above modified such that the plurality of separate containers comprises from two to ten separate containers.

In other embodiments, the invention provides the method described in any of the preceding paragraphs as applicable above modified such that the plurality of separate containers comprises from two to five separate containers.

In other embodiments, the invention provides the method described in any of the preceding paragraphs as applicable above modified such that the plurality of separate containers comprises 2, 3, 4, 5, 6, 7, 8, 9, 10, 11, 12, 13, 14, 15, 16, 17, 18, 19, or 20 separate containers.

In other embodiments, the invention provides the method described in any of the preceding paragraphs as applicable above modified such that for each container in which the priming first expansion is performed on a first population of TILs in step (b) the rapid second expansion in step (c) is performed in the same container on the second population of TILs produced from such first population of TILs.

In other embodiments, the invention provides the method described in any of the preceding paragraphs as applicable above modified such that each of the separate containers comprises a first gas-permeable surface area.

In other embodiments, the invention provides the method described in any of the preceding paragraphs as applicable above modified such that the multiple tumor fragments are distributed in a single container.

In other embodiments, the invention provides the method described in any of the preceding paragraphs as applicable above modified such that the single container comprises a first gas-permeable surface area.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In other embodiments, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In other embodiments, the invention provides the method described in any of the preceding paragraphs as applicable above modified such that in step (b) the APCs are layered onto the first gas-permeable surface area at an average thickness of at or about 2 cell layers.

In other embodiments, the invention provides the method described in any of the preceding paragraphs as applicable above modified such that in step (b) the APCs are layered onto the first gas-permeable surface area at an average thickness of at or about 1, 1.1, L2, 1.3, 1.4, 1.5, 1.6, 1.7, 1.8, L9, 2, 2.1, 2.2, 2.3, 2.4, 2.5, 2.6, 2.7, 2.8, 2.9 or 3 cell layers.

In other embodiments, the invention provides the method described in any of the preceding paragraphs as applicable above modified such that in step (c) the APCs are layered onto the first gas-permeable surface area at an average thickness of at or about 3 cell layers to at or about 10 cell layers.

In other embodiments, the invention provides the method described in any of the preceding paragraphs as applicable above modified such that in step (c) the APCs are layered onto the first gas-permeable surface area at an average thickness of at or about 4 cell layers to at or about 8 cell layers.

In other embodiments, the invention provides the method described in any of the preceding paragraphs as applicable above modified such that in step (c) the APCs are layered onto the first gas-permeable surface area at an average thickness of at or about 3, 4, 5, 6, 7, 8, 9 or 10 cell layers.

In other embodiments, the invention provides the method described in any of the preceding paragraphs as applicable above modified such that in step (c) the APCs are layered onto the first gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, the invention provides the method described in any of the preceding paragraphs as applicable above modified such that in step (b) the priming first expansion is performed in a first container comprising a first gas-permeable surface area and in step (c) the rapid second expansion is performed in a second container comprising a second gas-permeable surface area.

In other embodiments, the invention provides the method described in any of the preceding paragraphs as applicable above modified such that the second container is larger than the first container.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In other embodiments, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In other embodiments, the invention provides the method described in any of the preceding paragraphs as applicable above modified such that in step (b) the APCs are layered onto the first gas-permeable surface area at an average thickness of at or about 2 cell layers.

In other embodiments, the invention provides the method described in any of the preceding paragraphs as applicable modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In other embodiments, the invention provides the method described in any of the preceding paragraphs as applicable above modified such that in step (c) the APCs are layered onto the second gas-permeable surface area at an average thickness of at or about 3 cell layers to at or about 10 cell layers.

In other embodiments, the invention provides the method described in any of the preceding paragraphs as applicable above modified such that in step (c) the APCs are layered onto the second gas-permeable surface area at an average thickness of at or about 4 cell layers to at or about 8 cell layers.

In other embodiments, the invention provides the method described in any of the preceding paragraphs as applicable above modified such that in step (c) the APCs are layered onto the second gas-permeable surface area at an average thickness of at or about 3, 4, 5, 6, 7, 8, 9 or 10 cell layers.

In other embodiments, the invention provides the method described in any of the preceding paragraphs as applicable above modified such that in step (c) the APCs are layered onto the second gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, the invention provides the method described in any of the preceding paragraphs as applicable above modified such that in step (b) the priming first expansion is performed in a first container comprising a first gas-permeable surface area and in step (c) the rapid second expansion is performed in the first container.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In other embodiments, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In other embodiments, the invention provides the method described in any of the preceding paragraphs as applicable above modified such that in step (b) the APCs are layered onto the first gas-permeable surface area at an average thickness of at or about 2 cell layers.

In other embodiments, the invention provides the method described in any of the preceding paragraphs as applicable above modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In other embodiments, the invention provides the method described in any of the preceding paragraphs as applicable above modified such that in step (c) the APCs are layered onto the first gas-permeable surface area at an average thickness of at or about 3 cell layers to at or about 10 cell layers.

In other embodiments, the invention provides the method described in any of the preceding paragraphs as applicable above modified such that in step (c) the APCs are layered onto the first gas-permeable surface area at an average thickness of at or about 4 cell layers to at or about 8 cell layers.

In other embodiments, the invention provides the method described in any of the preceding paragraphs as applicable above modified such that in step (c) the APCs are layered onto the first gas-permeable surface area at an average thickness of at or about 3, 4, 5, 6, 7, 8, 9 or 10 cell layers.

In other embodiments, the invention provides the method described in any of the preceding paragraphs as applicable above modified such that in step (c) the APCs are layered onto the first gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:10.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:9.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:8.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:7,

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:6.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:4,

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:3.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:2.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.2 to at or about 1:8.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.3 to at or about 1:7.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.4 to at or about 1:6.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.5 to at or about 1:5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.6 to at or about 1:4.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.7 to at or about 1:3.5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.8 to at or about 1:3.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.9 to at or about 1:2.5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:2.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from at or about 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9 or 1:10.

In other embodiments, the invention provides the method described in any of preceding paragraphs as applicable above modified such that the ratio of the number of TILs in the second population of TILs to the number of TILs in the first population of TILs is at or about 1.5:1 to at or about 100:1.

In other embodiments, the invention provides the method described in any of the preceding paragraphs as applicable above modified such that the ratio of the number of TILs in the second population of TILs to the number of TILs in the first population of TILs is at or about 50:1.

In other embodiments, the invention provides the method described in any of the preceding paragraphs as applicable above modified such that the ratio of the number of TILs in the second population of TILs to the number of TILs in the first population of TILs is at or about 25:1.

In other embodiments, the invention provides the method described in any of the preceding paragraphs as applicable above modified such that the ratio of the number of TILs in the second population of TILs to the number of TILs in the first population of TILs is at or about 20:1.

In other embodiments, the invention provides the method described in any of the preceding paragraphs as applicable above modified such that the ratio of the number of TILs in the second population of TILs to the number of TILs in the first population of TILs is at or about 10:1.

In other embodiments, the invention provides the method described in any of the preceding paragraphs as applicable above modified such that the second population of TILs is at least at or about 50-fold greater in number than the first population of TILs.

In other embodiments, the invention provides the method described in any of the preceding paragraphs as applicable above modified such that the second population of TILs is at least at or about 1-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42- , 43-, 44-, 45-, 46-, 47-, 48-, 49- or 50-fold greater in number than the first population of TILs.

In other embodiments, the invention provides the method described in any of the preceding paragraphs as applicable above modified such that at or about 2 days or at or about 3 days after the commencement of the second period in step (c), the cell culture medium is supplemented with additional IL-2.

In other embodiments, the invention provides the method described in any of the preceding paragraphs as applicable above modified to further comprise the step of cryopreserving the harvested TIL population in step (d) using a cryopreservation process.

In other embodiments, the invention provides the method described in any of the preceding paragraphs as applicable above modified to comprise performing after step (d) the additional step of (e) transferring the harvested TIL population from step (d) to an infusion bag that optionally contains HypoThermosol.

In other embodiments, the invention provides the method described in any of the preceding paragraphs as applicable above modified to comprise the step of cryopreserving the infusion bag comprising the harvested TIL population in step (e) using a cryopreservation process.

In other embodiments, the invention provides the method described in any of the preceding paragraphs as applicable above modified such that the cryopreservation process is performed using a 1:1 ratio of harvested TIL population to cryopreservation media.

In other embodiments, the invention provides the method described in any of the preceding paragraphs as applicable above modified such that the antigen-presenting cells are peripheral blood mononuclear cells (PBMCs).

In other embodiments, the invention provides the method described in any of the preceding paragraphs as applicable above modified such that the PBMCs are irradiated and allogeneic.

In other embodiments, the invention provides the method described in any of the preceding paragraphs as applicable above modified such that the total number of APCs added to the cell culture in step (b) is 2.5×10⁸.

In other embodiments, the invention provides the method described in any of the preceding paragraphs as applicable above modified such that the total number of APCs added to the cell culture in step (c) is 5×10⁸.

In other embodiments, the invention provides the method described in any of the preceding paragraphs as applicable above modified such that the APCs are PBMCs.

In other embodiments, the invention provides the method described in any of the preceding paragraphs as applicable above modified such that the PBMCs are irradiated and allogeneic.

In other embodiments, the invention provides the method described in any of the preceding paragraphs as applicable above modified such that the antigen-presenting cells are artificial antigen-presenting cells.

In other embodiments, the invention provides the method described in any of the preceding paragraphs as applicable above modified such that the harvesting in step (d) is performed using a membrane-based cell processing system.

In other embodiments, the invention provides the method described in any of the preceding paragraphs as applicable above modified such that the harvesting in step (d) is performed using a LOVO cell processing system.

In other embodiments, the invention provides the method described in any of the preceding paragraphs as applicable above modified such that the multiple fragments comprise at or about 5 to at or about 60 fragments per container in step (b).

In other embodiments, the invention provides the method described in any of the preceding paragraphs as applicable above modified such that the multiple fragments comprise at or about 10 to at or about 60 fragments per container in step (b).

In other embodiments, the invention provides the method described in any of the preceding paragraphs as applicable above modified such that the multiple fragments comprise at or about 15 to at or about 60 fragments per container in step (b).

In other embodiments, the invention provides the method described in any of the preceding paragraphs as applicable above modified such that the multiple fragments comprise at or about 20 to at or about 60 fragments per container in step (b).

In other embodiments, the invention provides the method described in any of the preceding paragraphs as applicable above modified such that the multiple fragments comprise at or about 25 to at or about 60 fragments per container in step (b).

In other embodiments, the invention provides the method described in any of the preceding paragraphs as applicable above modified such that the multiple fragments comprise at or about 30 to at or about 60 fragments per container in step (b).

In other embodiments, the invention provides the method described in any of the preceding paragraphs as applicable above modified such that the multiple fragments comprise at or about 35 to at or about 60 fragments per container in step (b).

In other embodiments, the invention provides the method described in any of the preceding paragraphs as applicable above modified such that the multiple fragments comprise at or about 40 to at or about 60 fragments per container in step (b).

In other embodiments, the invention provides the method described in any of the preceding paragraphs as applicable above modified such that the multiple fragments comprise at or about 45 to at or about 60 fragments per container in step (b).

In other embodiments, the invention provides the method described in any of the preceding paragraphs as applicable above modified such that the multiple fragments comprise at or about 50 to at or about 60 fragments per container in step (b).

In other embodiments, the invention provides the method described in any of the preceding paragraphs as applicable above modified such that the multiple fragments comprise at or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 fragment(s) per container in step (b).

In other embodiments, the invention provides the method described in any of the preceding paragraphs as applicable above modified such that each fragment has a volume of at or about 27 mm³.

In other embodiments, the invention provides the method described in any of the preceding paragraphs as applicable above modified such that each fragment has a volume of at or about 20 mm³ to at or about 50 mm³.

In other embodiments, the invention provides the method described in any of the preceding paragraphs as applicable above modified such that each fragment has a volume of at or about 21 mm³ to at or about 30 mm³.

In other embodiments, the invention provides the method described in any of the preceding paragraphs as applicable above modified such that each fragment has a volume of at or about 22 mm³ to at or about 29.5 mm³.

In other embodiments, the invention provides the method described in any of the preceding paragraphs as applicable above modified such that each fragment has a volume of at or about 23 mm³ to at or about 29 mm³.

In other embodiments, the invention provides the method described in any of the preceding paragraphs as applicable above modified such that each fragment has a volume of at or about 24 mm³ to at or about 28.5 mm³.

In other embodiments, the invention provides the method described in any of the preceding paragraphs as applicable above modified such that each fragment has a volume of at or about 25 mm³ to at or about 28 mm³.

In other embodiments, the invention provides the method described in any of the preceding paragraphs as applicable above modified such that each fragment has a volume of at or about 26.5 mm³ to at or about 27.5 mm³.

In other embodiments, the invention provides the method described in any of the preceding paragraphs as applicable above modified such that each fragment has a volume of at or about 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mm³.

In other embodiments, the invention provides the method described in any of the preceding paragraphs as applicable above modified such that the multiple fragments comprise at or about 30 to at or about 60 fragments with a total volume of at or about 1300 mm³ to at or about 1500 mm³.

In other embodiments, the invention provides the method described in any of the preceding paragraphs as applicable above modified such that the multiple fragments comprise at or about 50 fragments with a total volume of at or about 1350 mm³.

In other embodiments, the invention provides the method described in any of the preceding paragraphs as applicable above modified such that the multiple fragments comprise at or about 50 fragments with a total mass of at or about 1 gram to at or about 1.5 grams.

In other embodiments, the invention provides the method described in any of the preceding paragraphs as applicable above modified such that the cell culture medium is provided in a container that is a G-container or a Xuri cellbag.

In other embodiments, the invention provides the method described in any of the preceding paragraphs as applicable above modified such that the IL-2 concentration in the cell culture medium is about 10,000 IU/mL to about 5,000 IU/mL.

In other embodiments, the invention provides the method described in any of the preceding paragraphs as applicable above modified such that the IL-2 concentration in the cell culture medium is about 6,000 IU/mL.

In other embodiments, the invention provides the method described in any of the preceding paragraphs as applicable above modified such that the cryopreservation media comprises dimethlysulfoxide (DMSO).

In other embodiments, the invention provides the method described in any of the preceding paragraphs as applicable above modified such that the cryopreservation media comprises 7% to 10% DMSO.

In other embodiments, the invention provides the method described in any of the preceding paragraphs as applicable above modified such that the first period in step (b) is performed within a period of at or about 1 day, 2 days, 3 days, 4 days, 5 days, 6 days, or 7 days.

In other embodiments, the invention provides the method described in any of the preceding paragraphs as applicable above modified such that the second period in step (c) is performed within a period of at or about 1 day, 2 days, 3 days, 4 days, 5 days, 6 days, 7 days, 8 days, 9 days, 10 days or 11 days.

In other embodiments, the invention provides the method described in any of the preceding paragraphs as applicable above modified such that the first period in step (b) and the second period in step (c) are each individually performed within a period of at or about 1 day, 2 days, 3 days, 4 days, 5 days, 6 days, or 7 days.

In other embodiments, the invention provides the method described in any of the preceding paragraphs as applicable above modified such that the first period in step (b) and the second period in step (c) are each individually performed within a period of at or about 5 days, 6 days, or 7 days.

In other embodiments, the invention provides the method described in any of the preceding paragraphs as applicable above modified such that the first period in step (b) and the second period in step (c) are each individually performed within a period of at or about 7 days.

In other embodiments, the invention provides the method described in any of the preceding paragraphs as applicable above modified such that steps (a) through (d) are performed in a total of at or about 14 days to at or about 18 days.

In other embodiments, the invention provides the method described in any of the preceding paragraphs as applicable above modified such that steps (a) through (d) are performed in a total of at or about 15 days to at or about 18 days.

In other embodiments, the invention provides the method described in any of the preceding paragraphs as applicable above modified such that steps (a) through (d) are performed in a total of at or about 16 days to at or about 18 days.

In other embodiments, the invention provides the method described in any of the preceding paragraphs as applicable above modified such that steps (a) through (d) are performed in a total of at or about 14 days to at or about 17 days.

In other embodiments, the invention provides the method described in any of the preceding paragraphs as applicable above modified such that steps (a) through (d) are performed in a total of at or about 15 days to at or about 17 days.

In other embodiments, the invention provides the method described in any of the preceding paragraphs as applicable above modified such that steps (a) through (d) are performed in a total of at or about 14 days to at or about 16 days.

In other embodiments, the invention provides the method described in any of the preceding paragraphs as applicable above modified such that steps (a) through (d) are performed in a total of at or about 15 days to at or about 16 days.

In other embodiments, the invention provides the method described in any of the preceding paragraphs as applicable above modified such that steps (a) through (d) are performed in a total of at or about 14 days.

In other embodiments, the invention provides the method described in any of the preceding paragraphs as applicable above modified such that steps (a) through (d) are performed in a total of at or about 15 days.

In other embodiments, the invention provides the method described in any of the preceding paragraphs as applicable above modified such that steps (a) through (d) are performed in a total of at or about 16 days.

In other embodiments, the invention provides the method described in any of the preceding paragraphs as applicable above modified such that steps (a) through (d) are performed in a total of at or about 17 days.

In other embodiments, the invention provides the method described in any of the preceding paragraphs as applicable above modified such that steps (a) through (d) are performed in a total of at or about 18 days.

In other embodiments, the invention provides the method described in any of the preceding paragraphs as applicable above modified such that steps (a) through (d) are performed in a total of at or about 14 days or less.

In other embodiments, the invention provides the method described in any of the preceding paragraphs as applicable above modified such that steps (a) through (d) are performed in a total of at or about 15 days or less.

In other embodiments, the invention provides the method described in any of the preceding paragraphs as applicable above modified such that steps (a) through (d) are performed in a total of at or about 16 days or less.

In other embodiments, the invention provides the method described in any of the preceding paragraphs as applicable above modified such that steps (a) through (d) are performed in a total of at or about 17 days or less.

In other embodiments, the invention provides the method described in any of the preceding paragraphs as applicable above modified such that steps (a) through (d) are performed in a total of at or about 18 days or less.

In other embodiments, the invention provides the method described in any of the preceding paragraphs as applicable above modified such that the therapeutic population of TILs harvested in step (d) comprises sufficient TILs for a therapeutically effective dosage of the TILs.

In other embodiments, the invention provides the method described in any of the preceding paragraphs as applicable above modified such that the number of TILs sufficient for a therapeutically effective dosage is from at or about 2.3×10¹⁰ to at or about 13.7×10¹⁰.

In other embodiments, the invention provides the method described in any of the preceding paragraphs as applicable above modified such that the third population of TILs in step (c) provides for increased efficacy, increased interferon-gamma production, and/or increased polyclonality.

In other embodiments, the invention provides the method described in any of the preceding paragraphs as applicable above modified such that the third population of TILs in step (c) provides for at least a one-fold to five-fold or more interferon-gamma production as compared to TILs prepared by a process longer than 16 days.

In other embodiments, the invention provides the method described in any of the preceding paragraphs as applicable above modified such that the third population of TILs in step (c) provides for at least a one-fold to five-fold or more interferon-gamma production as compared to TILs prepared by a process longer than 17 days.

In other embodiments, the invention provides the method described in any of the preceding paragraphs as applicable above modified such that the third population of TILs in step (c) provides for at least a one-fold to five-fold or more interferon-gamma production as compared to TILs prepared by a process longer than 18 days.

In other embodiments, the invention provides the method described in any of the preceding paragraphs as applicable above modified such that the effector T cells and/or central memory T cells obtained from the third population of TILs step (c) exhibit increased CD8 and CD28 expression relative to effector T cells and/or central memory T cells obtained from the second population of cells step (b).

In other embodiments, the invention provides the method described in any of the preceding paragraphs as applicable above modified such that each container recited in the method is a closed container.

In other embodiments, the invention provides the method described in any of the preceding paragraphs as applicable above modified such that each container recited in the method is a G-container.

In other embodiments, the invention provides the method described in any of the preceding paragraphs as applicable above modified such that each container recited in the method is a GREX-10.

In other embodiments, the invention provides the method described in any of the preceding paragraphs as applicable above modified such that each container recited in the method is a GREX-100.

In other embodiments, the invention provides the method described in any of the preceding paragraphs as applicable above modified such that each container recited in the method is a GREX-500.

In other embodiments, the invention provides the therapeutic population of tumor infiltrating lymphocytes (TILs) made by the method described in any of the preceding paragraphs as applicable above.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added antigen-presenting cells (APCs) or OKT3.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added antigen-presenting cells (APCs).

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added OKT3.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed with no added antigen-presenting cells (APCs) and no added OKT3.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 16 days.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 17 days.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 18 days.

In other embodiments, the invention provides for the therapeutic population of TILs described in any of the preceding paragraphs as applicable above that provides for increased interferon-gamma production.

In other embodiments, the invention provides for the therapeutic population of TILs described in any of the preceding paragraphs as applicable above that provides for increased polyclonality.

In other embodiments, the invention provides for the therapeutic population of TILs described in any of the preceding paragraphs as applicable above that provides for increased efficacy.

In other embodiments, the invention provides for the therapeutic population of TILs described in any of the preceding paragraphs as applicable above modified such that the therapeutic population of TILs is capable of at least one-fold more interferon-gamma production as compared to TILs prepared by a process longer than 16 days. In other embodiments, the invention provides for the therapeutic population of TILs described in any of the preceding paragraphs as applicable above modified such that the therapeutic population of TILs is capable of at least one-fold more interferon-gamma production as compared to TILs prepared by a process longer than 17 days. In other embodiments, the invention provides for the therapeutic population of TILs described in any of the preceding paragraphs as applicable above modified such that the therapeutic population of TILs is capable of at least one-fold more interferon-gamma production as compared to TILs prepared by a process longer than 18 days. In some embodiments, the TILs are rendered capable of the at least one-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for the therapeutic population of TILs described in any of the preceding paragraphs as applicable above modified such that the therapeutic population of TILs is capable of at least two-fold more interferon-gamma production as compared to TILs prepared by a process longer than 16 days. In other embodiments, the invention provides for the therapeutic population of TILs described in any of the preceding paragraphs as applicable above modified such that the therapeutic population of TILs is capable of at least two-fold more interferon-gamma production as compared to TILs prepared by a process longer than 17 days. In other embodiments, the invention provides for the therapeutic population of TILs described in any of the preceding paragraphs as applicable above modified such that the therapeutic population of TILs is capable of at least two-fold more interferon-gamma production as compared to TILs prepared by a process longer than 18 days. In some embodiments, the TILs are rendered capable of the at least two-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for the therapeutic population of TILs described in any of the preceding paragraphs as applicable above modified such that the therapeutic population of TILs is capable of at least three-fold more interferon-gamma production as compared to TILs prepared by a process longer than 16 days. In other embodiments, the invention provides for the therapeutic population of TILs described in any of the preceding paragraphs as applicable above modified such that the therapeutic population of TILs is capable of at least three-fold more interferon-gamma production as compared to TILs prepared by a process longer than 17 days. In other embodiments, the invention provides for the therapeutic population of TILs described in any of the preceding paragraphs as applicable above modified such that the therapeutic population of TILs is capable of at least three-fold more interferon-gamma production as compared to TILs prepared by a process longer than 18 days. In some embodiments, the TILs are rendered capable of the at least three-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for a therapeutic population of tumor infiltrating lymphocytes (TILs) that is capable of at least one-fold more interferon-gamma production as compared to TILs prepared by a process in which the first expansion of TILs is performed without any added antigen-presenting cells (APCs). In some embodiments, the TILs are rendered capable of the at least one-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for a therapeutic population of tumor infiltrating lymphocytes (TILs) that is capable of at least one-fold more interferon-gamma production as compared to TILs prepared by a process in which the first expansion of TILs is performed without any added OKT3. In some embodiments, the TILs are rendered capable of the at least one-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for a therapeutic population of TILs that is capable of at least two-fold more interferon-gamma production as compared to TILs prepared by a process in which the first expansion of TILs is performed without any added APCs.

In other embodiments, the invention provides for a therapeutic population of TILs that is capable of at least two-fold more interferon-gamma production as compared to TILs prepared by a process in which the first expansion of TILs is performed without any added APCs. In some embodiments, the TILs are rendered capable of the at least two-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for a therapeutic population of TILs that is capable of at least two-fold more interferon-gamma production as compared to TILs prepared by a process in which the first expansion of TILs is performed without any added OKT3. In some embodiments, the TILs are rendered capable of the at least two-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for a therapeutic population of TILs that is capable of at least three-fold more interferon-gamma production as compared to TILs prepared by a process in which the first expansion of TILs is performed without any added APCs. In some embodiments, the TILs are rendered capable of the at least one-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides for a therapeutic population of TILs that is capable of at least three-fold more interferon-gamma production as compared to TILs prepared by a process in which the first expansion of TILs is performed without any added OKT3. In some embodiments, the TILs are rendered capable of the at least three-fold more interferon-gamma production due to the expansion process described herein, for example as described in Steps A through F above or according to Steps A through F above (also as shown, for example, in FIG. 1 (in particular, e.g., FIG. 1B and/or FIG. 1C and/or FIG. 1D and/or FIG. 1E and/or FIG. 1F and/or FIG. 1G and/or FIG. 1H).

In other embodiments, the invention provides a method of expanding T cells comprising: (a) performing a priming first expansion of a first population of T cells obtained from a donor by culturing the first population of T cells to effect growth and to prime an activation of the first population of T cells; (b) after the activation of the first population of T cells primed in step (a) begins to decay, performing a rapid second expansion of the first population of T cells by culturing the first population of T cells to effect growth and to boost the activation of the first population of T cells to obtain a second population of T cells; and (c) harvesting the second population of T cells. In other embodiments, the step of rapid second expansion is split into a plurality of steps to achieve a scaling up of the culture by: (a) performing the rapid second expansion by culturing the first population of T cells in a small scale culture in a first container, e.g., a G-REX 100MCS container, for a period of about 3 to 4 days, and then (b) effecting the transfer of the first population of T cells from the small scale culture to a second container larger than the first container, e.g., a G-REX 500MCS container, and culturing the first population of T cells from the small scale culture in a larger scale culture in the second container for a period of about 4 to 7 days. In other embodiments, the step of rapid expansion is split into a plurality of steps to achieve a scaling out of the culture by: (a) performing the rapid second expansion by culturing the first population of T cells in a first small scale culture in a first container, e.g., a G-REX 100MCS container, for a period of about 3 to 4 days, and then (b) effecting the transfer and apportioning of the first population of T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the first population of T cells from first small scale culture transferred to such second container is cultured in a second small scale culture for a period of about 4 to 7 days. In other embodiments, the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the first population of T cells from the small scale culture transferred to such second container is cultured in a larger scale culture for a period of about 4 to 7 days. In other embodiments, the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 5 days.

In other embodiments, the invention provides the method described in any of the preceding paragraphs as applicable above modified such that the step of rapid second expansion is split into a plurality of steps to achieve a scaling up of the culture by: (a) performing the rapid second expansion by culturing the first population of T cells in a small scale culture in a first container, e.g., a G-REX 100MCS container, for a period of about 2 to 4 days, and then (b) effecting the transfer of the first population of T cells from the small scale culture to a second container larger than the first container, e.g., a G-REX 500MCS container, and culturing the first population of T cells from the small scale culture in a larger scale culture in the second container for a period of about 5 to 7 days.

In other embodiments, the invention provides the method described in any of the preceding paragraphs as applicable above modified such that the step of rapid expansion is split into a plurality of steps to achieve a scaling out of the culture by: (a) performing the rapid second expansion by culturing the first population of T cells in a first small scale culture in a first container, e.g., a G-REX 100MCS container, for a period of about 2 to 4 days, and then (b) effecting the transfer and apportioning of the first population of T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the first population of T cells from first small scale culture transferred to such second container is cultured in a second small scale culture for a period of about 5 to 7 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 2 to 4 days, and then (b) effecting the transfer and apportioning of the first population of T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the first population of T cells from the small scale culture transferred to such second container is cultured in a larger scale culture for a period of about 5 to 7 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 5 to 6 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 5 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 6 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 7 days.

In other embodiments, the invention provides the method described in any of the preceding paragraphs as applicable above modified such that the priming first expansion of step (a) is performed during a period of up to 7 days.

In other embodiments, the invention provides the method described in any of the preceding paragraphs as applicable above modified such that the rapid second expansion of step (b) is performed during a period of up to 8 days.

In other embodiments, the invention provides the method described in any of the preceding paragraphs as applicable above modified such that the rapid second expansion of step (b) is performed during a period of up to 9 days.

In other embodiments, the invention provides the method described in any of the preceding paragraphs as applicable above modified such that the rapid second expansion of step (b) is performed during a period of up to 10 days.

In other embodiments, the invention provides the method described in any of the preceding paragraphs as applicable above modified such that the rapid second expansion of step (b) is performed during a period of up to 11 days.

In other embodiments, the invention provides the method described in any of the preceding paragraphs as applicable above modified such that the priming first expansion in step (a) is performed during a period of 7 days and the rapid second expansion of step (b) is performed during a period of up to 9 days.

In other embodiments, the invention provides the method described in any of the preceding paragraphs as applicable above modified such that the priming first expansion in step (a) is performed during a period of 7 days and the rapid second expansion of step (b) is performed during a period of up to 10 days.

In other embodiments, the invention provides the method described in any of the preceding paragraphs as applicable above modified such that the priming first expansion in step (a) is performed during a period of 7 days or 8 days and the rapid second expansion of step (b) is performed during a period of up to 9 days.

In other embodiments, the invention provides the method described in any of the preceding paragraphs as applicable above modified such that the priming first expansion in step (a) is performed during a period of 7 days or 8 days and the rapid second expansion of step (b) is performed during a period of up to 10 days.

In other embodiments, the invention provides the method described in any of the preceding paragraphs as applicable above modified such that the priming first expansion in step (a) is performed during a period of 8 days and the rapid second expansion of step (b) is performed during a period of up to 9 days.

In other embodiments, the invention provides the method described in any of the preceding paragraphs as applicable above modified such that the priming first expansion in step (a) is performed during a period of 8 days and the rapid second expansion of step (b) is performed during a period of up to 8 days.

In other embodiments, the invention provides the method described in any of the preceding paragraphs as applicable above modified such that in step (a) the first population of T cells is cultured in a first culture medium comprising OKT-3 and IL-2.

In other embodiments, the invention provides the method described in any of the preceding paragraphs as applicable above modified such that the first culture medium comprises 4-1BB agonist, OKT-3 and IL-2.

In other embodiments, the invention provides the method described in any of the preceding paragraphs as applicable above modified such that the first culture medium comprises OKT-3, IL-2 and antigen-presenting cells (APCs).

In other embodiments, the invention provides the method described in any of the preceding paragraphs as applicable above modified such that the first culture medium comprises 4-1BB agonist, OKT-3, IL-2 and antigen-presenting cells (APCs).

In other embodiments, the invention provides the method described in any of the preceding paragraphs as applicable above modified such that in step (a) the first population of T cells is cultured in a first culture medium in a container comprising a first gas-permeable surface, wherein the first culture medium comprises OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that in step (a) the first population of T cells is cultured in a first culture medium in a container comprising a first gas-permeable surface, wherein the first culture medium comprises 4-1BB agonist, OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that in step (a) the first population of T cells is cultured in a first culture medium in a container comprising a first gas-permeable surface, wherein the first culture medium comprises OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises 4-1BB agonist, OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that in step (a) the first population of T cells is cultured in a first culture medium in a container comprising a first gas-permeable surface, wherein the first culture medium comprises 4-1BB agonist, OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises 4-1BB agonist, OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that the ratio of the number of APCs in the second population of APCs to the number of APCs in the first population of APCs is about 2:1.

In other embodiments, the invention provides the method described in any of the preceding paragraphs as applicable above modified such that the number of APCs in the first population of APCs is about 2.5×10⁸ and the number of APCs in the second population of APCs is about 5×10⁸.

In other embodiments, the invention provides the method described in any of the preceding paragraphs as applicable above modified such that in step (a) the first population of APCs is layered onto the first gas-permeable surface at an average thickness of 2 layers of APCs.

In other embodiments, the invention provides the method described in any of the preceding paragraphs as applicable above modified such that in step (b) the second population of APCs is layered onto the first gas-permeable surface at an average thickness selected from the range of 4 to 8 layers of APCs.

In other embodiments, the invention provides the method described in any of the preceding paragraphs as applicable above modified such that the ratio of the average number of layers of APCs layered onto the first gas-permeable surface in step (b) to the average number of layers of APCs layered onto the first gas-permeable surface in step (a) is 2:1.

In other embodiments, the invention provides the method described in any of the preceding paragraphs as applicable above modified such that in step (a) the first population of APCs is seeded on the first gas permeable surface at a density selected from the range of at or about 1.0×10⁶ APCs/cm² to at or about 4.5×10⁶ APCs/cm².

In other embodiments, the invention provides the method described in any of the preceding paragraphs as applicable above modified such that in step (a) the first population of APCs is seeded on the first gas permeable surface at a density selected from the range of at or about 1.5×10⁶ APCs/cm² to at or about 3.5×10⁶ APCs/cm².

In other embodiments, the invention provides the method described in any of the preceding paragraphs as applicable above modified such that in step (a) the first population of APCs is seeded on the first gas permeable surface at a density selected from the range of at or about 2.0×10⁶ APCs/cm² to at or about 3.0×10⁶ APCs/cm².

In other embodiments, the invention provides the method described in any of the preceding paragraphs as applicable above modified such that in step (a) the first population of APCs is seeded on the first gas permeable surface at a density of at or about 2.0×10⁶ APCs/cm².

In other embodiments, the invention provides the method described in any of the preceding paragraphs as applicable above modified such that in step (b) the second population of APCs is seeded on the first gas permeable surface at a density selected from the range of at or about 2.5×10⁶ APCs/cm² to at or about 7.5×10⁶ APCs/cm².

In other embodiments, the invention provides the method described in any of the preceding paragraphs as applicable above modified such that in step (b) the second population of APCs is seeded on the first gas permeable surface at a density selected from the range of at or about 3.5×10⁶ APCs/cm² to at or about 6.0×10⁶ APCs/cm².

In other embodiments, the invention provides the method described in any of the preceding paragraphs as applicable above modified such that in step (b) the second population of APCs is seeded on the first gas permeable surface at a density selected from the range of at or about 4.0×10⁶ APCs/cm² to at or about 5.5×10⁶ APCs/cm².

In other embodiments, the invention provides the method described in any of the preceding paragraphs as applicable above modified such that in step (b) the second population of APCs is seeded on the first gas permeable surface at a density of at or about 4.0×10⁶ APCs/cm².

In other embodiments, the invention provides the method described in any of the preceding paragraphs as applicable above modified such that in step (a) the first population of APCs is seeded on the first gas permeable surface at a density selected from the range of at or about 1.0×10⁶ APCs/cm² to at or about 4.5×10⁶ APCs/cm² and in step (b) the second population of APCs is seeded on the first gas permeable surface at a density selected from the range of at or about 2.5×10⁶ APCs/cm² to at or about 7.5×10⁶ APCs/cm².

In other embodiments, the invention provides the method described in any of the preceding paragraphs as applicable modified such that in step (a) the first population of APCs is seeded on the first gas permeable surface at a density selected from the range of at or about 1.5×10⁶ APCs/cm² to at or about 3.5×10⁶ APCs/cm² and in step (b) the second population of APCs is seeded on the first gas permeable surface at a density selected from the range of at or about 3.5×10⁶ APCs/cm² to at or about 6.0×10⁶ APCs/cm².

In other embodiments, the invention provides the method described in any of the preceding paragraphs as applicable above modified such that in step (a) the first population of APCs is seeded on the first gas permeable surface at a density selected from the range of at or about 2.0×10⁶ APCs/cm² to at or about 3.0×10⁶ APCs/cm² and in step (b) the second population of APCs is seeded on the first gas permeable surface at a density selected from the range of at or about 4.0×10⁶ APCs/cm² to at or about 5.5×10⁶ APCs/cm².

In other embodiments, the invention provides the method described in any of the preceding paragraphs as applicable above modified such that in step (a) the first population of APCs is seeded on the first gas permeable surface at a density of at or about 2.0×10⁶ APCs/cm² and in step (b) the second population of APCs is seeded on the first gas permeable surface at a density of at or about 4.0×10⁶ APCs/cm².

In other embodiments, the invention provides the method described in any of the preceding paragraphs as applicable above modified such that the APCs are peripheral blood mononuclear cells (PBMCs).

In other embodiments, the invention provides the method described in any of the preceding paragraphs as applicable above modified such that the PBMCs are irradiated and exogenous to the donor of the first population of T cells.

In other embodiments, the invention provides the method described in any of the preceding paragraphs as applicable above modified such that the T cells are tumor infiltrating lymphocytes (TILs).

In other embodiments, the invention provides the method described in any of the preceding paragraphs as applicable above modified such that the T cells are marrow infiltrating lymphocytes (MILs).

In other embodiments, the invention provides the method described in any of the preceding paragraphs as applicable above modified such that the T cells are peripheral blood lymphocytes (PBLs).

In other embodiments, the invention provides the method described in any of the preceding paragraphs as applicable above modified such that the first population of T cells is obtained by separation from the whole blood of the donor.

In other embodiments, the invention provides the method described in any of the preceding paragraphs as applicable above modified such that the first population of T cells is obtained by separation from the apheresis product of the donor.

In other embodiments, the invention provides the method described in any of the preceding paragraphs as applicable above modified such that the first population of T cells is separated from the whole blood or apheresis product of the donor by positive or negative selection of a T cell phenotype.

In other embodiments, the invention provides the method described in any of the preceding paragraphs as applicable above modified such that the T cell phenotype is CD3+ and CD45+.

In other embodiments, the invention provides the method described in any of the preceding paragraphs as applicable above modified such that before performing the priming first expansion of the first population of T cells the T cells are separated from NK cells. In other embodiments, the T cells are separated from NK cells in the first population of T cells by removal of CD3− CD56+ cells from the first population of T cells. In other embodiments, the CD3− CD56+ cells are removed from the first population of T cells by subjecting the first population of T cells to cell sorting using a gating strategy that removes the CD3− CD56+ cell fraction and recovers the negative fraction. In other embodiments, the foregoing method is utilized for the expansion of T cells in a first population of T cells characterized by a high percentage of NK cells. In other embodiments, the foregoing method is utilized for the expansion of T cells in a first population of T cells characterized by a high percentage of CD3− CD56+ cells. In other embodiments, the foregoing method is utilized for the expansion of T cells in tumor tissue characterized by the present of a high number of NK cells. In other embodiments, the foregoing method is utilized for the expansion of T cells in tumor tissue characterized by a high number of CD3− CD56+ cells. In other embodiments, the foregoing method is utilized for the expansion of T cells in tumor tissue obtained from a patient suffering from a tumor characterized by the presence of a high number of NK cells. In other embodiments, the foregoing method is utilized for the expansion of T cells in tumor tissue obtained from a patient suffering from a tumor characterized by the presence of a high number of CD3− CD56+ cells. In other embodiments, the foregoing method is utilized for the expansion of T cells in tumor tissue obtained from a patient suffering from ovarian cancer.

In other embodiments, the invention provides the method described in any of the preceding paragraphs as applicable above modified such that at or about 1×10⁷ T cells from the first population of T cells are seeded in a container to initiate the primary first expansion culture in such container.

In other embodiments, the invention provides the method described in any of the preceding paragraphs as applicable above modified such that the first population of T cells is distributed into a plurality of containers, and in each container at or about 1×10⁷ T cells from the first population of T cells are seeded to initiate the primary first expansion culture in such container.

In other embodiments, the invention provides the method described in any of the preceding paragraphs as applicable above modified such that the second population of T cells harvested in step (c) is a therapeutic population of TILs.

III. Pharmaceutical Compositions, Dosages, and Dosing Regimens

In some embodiments, TILs expanded using the methods of the present disclosure are administered to a patient as a pharmaceutical composition. In some embodiments, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is melanoma. In some embodiments, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, the therapeutically effective dosage is about 2.3×10¹⁰ to about 13.7×10¹⁰. In some embodiments, the therapeutically effective dosage is about 7.8×10¹⁰ TILs, particularly of the cancer is melanoma. In some embodiments, the therapeutically effective dosage is about 1.2×10¹⁰ to about 4.3×10¹⁰ of TILs. In some embodiments, the therapeutically effective dosage is about 3×10¹⁰ to about 12×10¹⁰ TILs. In some embodiments, the therapeutically effective dosage is about 4×10¹⁰ to about 10×10¹⁰ TILs. In some embodiments, the therapeutically effective dosage is about 5×10¹⁰ to about 8×10¹⁰ TILs. In some embodiments, the therapeutically effective dosage is about 6×10¹⁰ to about 8×10¹⁰ TILs. In some embodiments, the therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0,005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 1425% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 025 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

In other embodiments, the invention provides an infusion bag comprising the therapeutic population of TILs described in any of the preceding paragraphs as applicable above.

In other embodiments, the invention provides a tumor infiltrating lymphocyte (TIL) composition comprising the therapeutic population of TILs described in any of the preceding paragraphs as applicable above and a pharmaceutically acceptable carrier.

In other embodiments, the invention provides an infusion bag comprising the TIL composition described in any of the preceding paragraphs as applicable above.

In other embodiments, the invention provides a cryopreserved preparation of the therapeutic population of TILs described in any of the preceding paragraphs as applicable above.

In other embodiments, the invention provides a tumor infiltrating lymphocyte (TIL) composition comprising the therapeutic population of TILs described in any of the preceding paragraphs as applicable above and a cryopreservation media.

In other embodiments, the invention provides the TIL composition described in any of the preceding paragraphs as applicable above modified such that the cryopreservation media contains DMSO.

In other embodiments, the invention provides the TIL composition described in any of the preceding paragraphs as applicable above modified such that the cryopreservation media contains 7-10% DMSO.

In some embodiments, the invention provides the TIL compositions comprising TILs in serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium CTS™ OpTmizer™ T-Cell Expansion SFM, CTS™ AIM-V Medium, CTS™ A1M-V SFM, LymphoONE™ T-Cell Expansion Xeno-Free Medium, Dulbecco's Modified Eagle's Medium (DMEM), Minimal Essential Medium (MEM), Basal Medium Eagle (BME). RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³″, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L histidine, L-isoleucine, L-methionine, L-phenylalanine, L-proline, hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³″, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BML), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1X Medium” in Table 4. In other embodiments, the non-trace element moiety ingredients in the defined medium are present in the final concentrations listed in the column under the heading “A Preferred Embodiment of the 1X Medium” in Table 4. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 4.

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., “Ex vivo expansion of human T cells for adoptive immunotherapy using the novel Xeno-free CTS Immune Cell Serum Replacement,” Clin Transl Immunology, 4 (1) 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or βME; also known as 2-mercaptoethanol, CAS 60-24-2).

In other embodiments, the invention provides a cryopreserved preparation of the TIL composition described in any of the preceding paragraphs as applicable.

IV. Methods of Treating Patients

Methods of treatment begin with the initial TIL collection and culture of TILs. Such methods have been both described in the art by, for example, Jin et al., J. Immunotherapy, 2012, 35(3):283-292, incorporated by reference herein in its entirety. Embodiments of methods of treatment are described throughout the sections below, including the Examples.

The expanded TILs produced according the methods described herein, including for example as described in Steps A through F above or according to Steps A through F above (also as shown, for example, in FIG. 1 (in particular, e.g., FIG. 1B) find particular use in the treatment of patients with cancer (for example, as described in Goff, et al., J. Clinical Oncology, 2016, 34(20):2389-239, as well as the supplemental content; incorporated by reference herein in its entirety. In some embodiments, TIL were grown from resected deposits of metastatic melanoma as previously described (see, Dudley, et al., J Immunother., 2003, 26:332-342; incorporated by reference herein in its entirety). Fresh tumor can be dissected under sterile conditions. A representative sample can be collected for formal pathologic analysis. Single fragments of 2 mm³ to 3 mm³ may be used. In some embodiments, 5, 10, 15, 20, 25 or 30 samples per patient are obtained. In some embodiments, 20, 25, or 30 samples per patient are obtained. In some embodiments, 20, 22, 24, 26, or 28 samples per patient are obtained. In some embodiments, 24 samples per patient are obtained. Samples can be placed in individual wells of a 24-well plate, maintained in growth media with high-dose IL-2 (6,000 IU/mL), and monitored for destruction of tumor and/or proliferation of TIL. Any tumor with viable cells remaining after processing can be enzymatically digested into a single cell suspension and cryopreserved, as described herein.

In some embodiments, successfully grown TIL can be sampled for phenotype analysis (CD3, CD4, CD8, and CD56) and tested against autologous tumor when available. TIL can be considered reactive if overnight coculture yielded interferon-gamma (IFN-γ) levels >200 pg/mL and twice background. (Goff, et al., J Immunother., 2010, 33:840-847; incorporated by reference herein in its entirety). In some embodiments, cultures with evidence of autologous reactivity or sufficient growth patterns can be selected for a second expansion (for example, a second expansion as provided in according to Step D of FIG. 1 (in particular, e.g., FIG. 1B), including second expansions that are sometimes referred to as rapid expansion (REP). In some embodiments, expanded TILs with high autologous reactivity (for example, high proliferation during a second expansion), are selected for an additional second expansion. In some embodiments, TILs with high autologous reactivity (for example, high proliferation during second expansion as provided in Step D of FIG. 1 (in particular, e.g., FIG. 1B), are selected for an additional second expansion according to Step D of FIG. 1 (in particular, e.g., FIG. 1B).

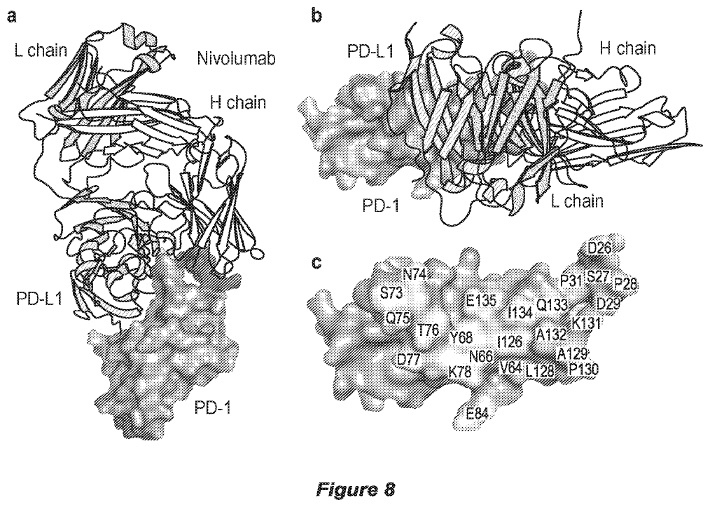

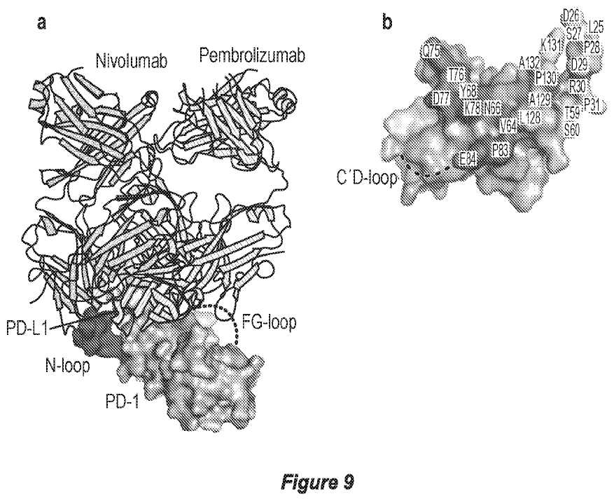

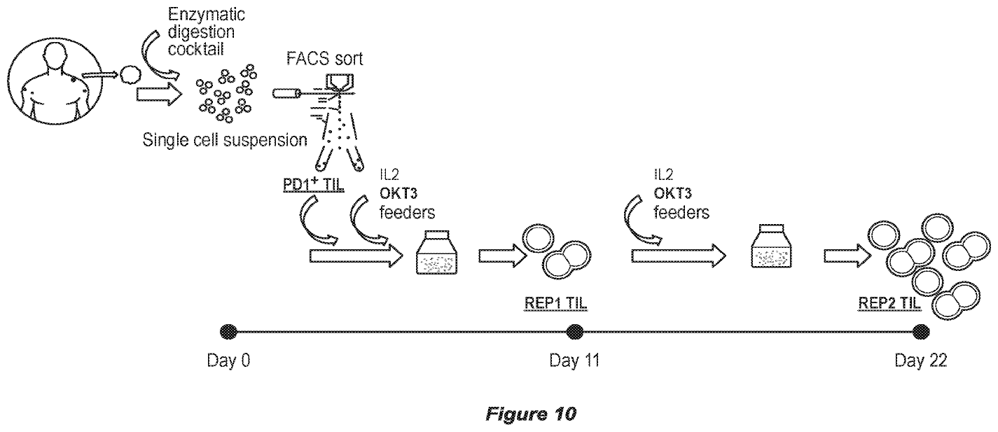

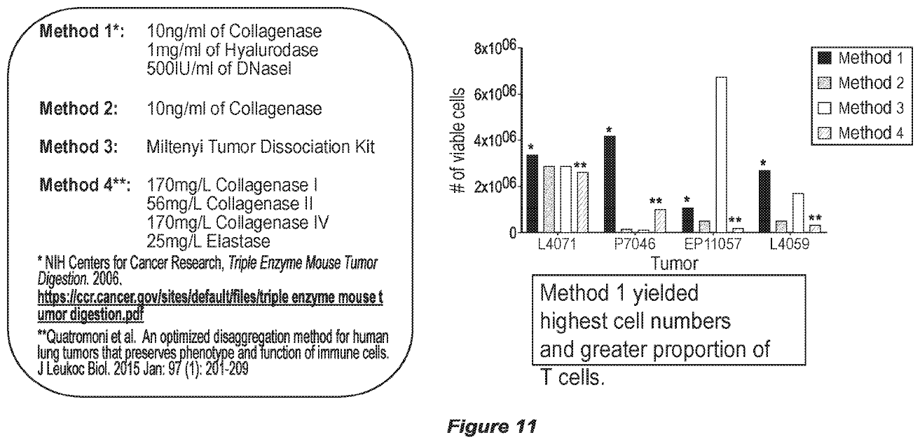

In some embodiments, IFN-gamma (IFN-γ) is indicative of treatment efficacy and/or increased clinical efficacy. In some embodiments, IFN-γ in the blood of subjects treated with TILs is indicative of active TILs. In some embodiments, a potency assay for IFN-γ production is employed. IFN-γ production is another measure of cytotoxic potential. IFN-γ production can be measured by determining the levels of the cytokine IFN-γ in the blood, serum, or TILs ex vivo of a subject treated with TILs prepared by the methods of the present invention, including those as described for example in FIG. 1 and/or FIG. 8. In some embodiments, an increase in IFN-γ is indicative of treatment efficacy in a patient treated with the TILs produced by the methods of the present invention. In some embodiments, IFN-γ is increased one-fold, two-fold, three-fold, four-fold, or five-fold or more IFN-γ as compared to an untreated patient and/or as compared to a patient treated with TILs prepared using other methods than those provide herein including for example, methods other than those embodied in FIG. 1 and/or FIG. 8.

In some embodiments, the patient is not moved directly to ACT (adoptive cell transfer), for example, in some embodiments, after tumor harvesting and/or a first expansion, the cells are not utilized immediately. In some embodiments, TILs can be cryopreserved and thawed 2 days before administration to a patient. In some embodiments, TILs can be cryopreserved and thawed 1 day before administration to a patient. In some embodiments, the TILs can be cryopreserved and thawed immediately before the administration to a patient.

Cell phenotypes of cryopreserved samples of infusion bag TIL can be analyzed by flow cytometry (e.g., FlowJo) for surface markers CD3, CD4, CD8, CCR7, and CD45RA (BD BioSciences), as well as by any of the methods described herein. Serum cytokines were measured by using standard enzyme-linked immunosorbent assay techniques. A rise in serum IFN-g was defined as >100 pg/mL and greater than 4 3 baseline levels.

In some embodiments, the TILs produced by the methods provided herein, for example those exemplified in FIG. 1 (in particular, e.g., FIG. 1B), provide for a surprising improvement in clinical efficacy of the TILs. In some embodiments, the TILs produced by the methods provided herein, for example those exemplified in FIG. 1 (in particular, e.g., FIG. 1B), exhibit increased clinical efficacy as compared to TILs produced by methods other than those described herein, including for example, methods other than those exemplified in FIG. 1 (in particular, e.g., FIG. 1B). In some embodiments, the methods other than those described herein include methods referred to as process 1C and/or Generation 1 (Gen 1). In some embodiments, the increased efficacy is measured by DCR, ORR, and/or other clinical responses. In some embodiments, the TILs produced by the methods provided herein, for example those exemplified in FIG. 1 (in particular, e.g., FIG. 1B), exhibit a similar time to response and safety profile compared to TILs produced by methods other than those described herein, including for example, methods other than those exemplified in FIG. 1 (in particular, e.g., FIG. 1B), for example the Gen 1 process.

In some embodiments, IFN-gamma (IFN-γ) is indicative of treatment efficacy and/or increased clinical efficacy. In some embodiments, IFN-γ in the blood of subjects treated with TILs is indicative of active TILs. In some embodiments, a potency assay for IFN-γ production is employed. IFN-γ production is another measure of cytotoxic potential. IFN-γ production can be measured by determining the levels of the cytokine IFN-γ in the blood, serum, or TILs ex vivo of a subject treated with TILs prepared by the methods of the present invention, including those as described for example in FIG. 1 (in particular, e.g., FIG. 1B). In some embodiments, an increase in IFN-γ is indicative of treatment efficacy in a patient treated with the TILs produced by the methods of the present invention. In some embodiments, IFN-γ is increased one-fold, two-fold, three-fold, four-fold, or five-fold or more as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased one-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased two-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased three-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased four-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased five-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ is measured using a Quantikine ELISA kit. In some embodiments, IFN-γ is measured in TILs ex vivo of a subject treated with TILs prepared by the methods of the present invention, including those as described for example in FIG. 1 (in particular, e.g., FIG. 1B). In some embodiments, IFN-γ is measured in blood of a subject treated with TILs prepared by the methods of the present invention, including those as described for example in FIG. 1 (in particular, e.g., FIG. 1B). In some embodiments, IFN-γ is measured in TILs serum of a subject treated with TILs prepared by the methods of the present invention, including those as described for example in FIG. 1 (in particular, e.g., FIG. 1B).

In some embodiments, the TILs prepared by the methods of the present invention, including those as described for example in FIG. 1 (in particular, e.g., FIG. 1B), exhibit increased polyclonality as compared to TILs produced by other methods, including those not exemplified in FIG. 1 (in particular, e.g., FIG. 1B), such as for example, methods referred to as process 1C methods. In some embodiments, significantly improved polyclonality and/or increased polyclonality is indicative of treatment efficacy and/or increased clinical efficacy. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1 (in particular, e.g., FIG. 1B). In some embodiments, polyclonality is increased one-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased two-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased ten-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased 100-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased 500-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased 1000-fold as compared to an untreated patient and/or as compared to a patient treated with TILs prepared using other methods than those provide herein including for example, methods other than those embodied in FIG. 1 (in particular, e.g., FIG. 1B).

Measures of efficacy can include the disease control rate (DCR) as well as overall response rate (ORR), as known in the art as well as described herein.

1. Methods of Treating Cancers and Other Diseases

The compositions and methods described herein can be used in a method for treating diseases. In some embodiments, they are for use in treating hyperproliferative disorders. They may also be used in treating other disorders as described herein and in the following paragraphs.

In some embodiments, the hyperproliferative disorder is cancer. In some embodiments, the hyperproliferative disorder is a solid tumor cancer. In some embodiments, the solid tumor cancer is selected from the group consisting of glioblastoma (GBM), gastrointestinal cancer, melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), colon, colorectal, pancreatic, triple negative breast cancer (TNBC), sarcoma, renal cancer, and renal cell carcinoma. In some embodiments, the hyperproliferative disorder is a hematological malignancy. In some embodiments, the solid tumor cancer is selected from the group consisting of chronic lymphocytic leukemia, acute lymphoblastic leukemia, diffuse large B cell lymphoma, non-Hodgkin's lymphoma, Hodgkin's lymphoma, follicular lymphoma, and mantle cell lymphoma.

In some embodiments, the cancer is a hypermutated cancer phenotype. Hypermutated cancers are extensively described in Campbell, et al. (Cell, 171:1042-1056 (2017); incorporated by reference herein in its entirety for all purposes). In some embodiments, a hypermutated tumors comprise between 9 and 10 mutations per megabase (Mb). In some embodiments, pediatric hypermutated tumors comprise 9.91 mutations per megabase (Mb). In some embodiments, adult hypermutated tumors comprise 9 mutations per megabase (Mb). In some embodiments, enhanced hypermutated tumors comprise between 10 and 100 mutations per megabase (Mb). In some embodiments, enhanced pediatric hypermutated tumors comprise between 10 and 100 mutations per megabase (Mb). In some embodiments, enhanced adult hypermutated tumors comprise between 10 and 100 mutations per megabase (Mb). In some embodiments, an ultra-hypermutated tumors comprise greater than 100 mutations per megabase (Mb). In some embodiments, pediatric ultra-hypermutated tumors comprise greater than 100 mutations per megabase (Mb). In some embodiments, adult ultra-hypermutated tumors comprise greater than 100 mutations per megabase (Mb).

In some embodiments, the hypermutated tumors have mutations in replication repair pathways. In some embodiments, the hypermutated tumors have mutations in replication repair associated DNA polymerases. In some embodiments, the hypermutated tumors have microsatellite instability. In some embodiments, the ultra-hypermutated tumors have mutations in replication repair associated DNA polymerases and have microsatellite instability. In some embodiments, hypermutation in the tumor is correlated with response to immune checkpoint inhibitors. In some embodiments, hypermutated tumors are resistant to treatment with immune checkpoint inhibitors. In some embodiments, hypermutated tumors can be treated using the TILs of the present invention. In some embodiments, hypermutation in the tumor is caused by environmental factors (extrinsic exposures). For example, UV light can be the primary cause of the high numbers of mutations in malignant melanoma (see, for example, Pfeifer, G. P., You, Y. H., and Besaratinia, A. (2005). Mutat. Res. 571, 19-31.; Sage, E. (1993). Photochem. Photobiol. 57, 163-174.). In some embodiments, hypermutation in the tumor can be caused by the greater than 60 carcinogens in tobacco smoke for tumors of the lung and larynx, as well as other tumors, due to direct mutagen exposure (see, for example, Pleasance, E. D., Stephens, P. J., O'Meara, S., McBride, D. J., Meynert, A., Jones, D., Lin, M. L., Beare, D., Lau, K. W., Greenman, C., et al. (2010). Nature 463, 184-190). In some embodiments, hypermutation in the tumor is caused by dysregulation of apolipoprotein B mRNA editing enzyme, catalytic polypeptide-like (APOBEC) family members, which has been shown to result in increased levels of C to T transitions in a wide range of cancers (see, for example, Roberts, S. A., Lawrence, M. S., Klimczak, L. J., Grimm, S. A., Fargo, D., Stojanov, P., Kiezun, A., Kryukov, G. V., Carter, S. L., Saksena, G., et al. (2013). Nat. Genet. 45, 970-976). In some embodiments, hypermutation in the tumor is caused by defective DNA replication repair by mutations that compromise proofreading, which is performed by the major replicative enzymes Pol3 and Pold1. In some embodiments, hypermutation in the tumor is caused by defects in DNA mismatch repair, which are associated with hypermutation in colorectal, endometrial, and other cancers (see, for example, Kandoth, C., Schultz, N., Cherniack, A. D., Akbani, R., Liu, Y., Shen, H., Robertson, A. G., Pashtan, I., Shen, R., Benz, C. C., et al.; (2013). Nature 497, 67-73.; Muzny, D. M., Bainbridge, M. N., Chang, K., Dinh, H. H., Drummond, J. A., Fowler, G., Kovar, C. L., Lewis, L. R., Morgan, M. B., Newsham, I. F., et al.; (2012). Nature 487, 330-337). In some embodiments, DNA replication repair mutations are also found in cancer predisposition syndromes, such as constitutional or biallelic mismatch repair deficiency (CMMRD), Lynch syndrome, and polymerase proofreading-associated polyposis (PPAP).

In some embodiments, the invention includes a method of treating a cancer with a population of TILs, wherein the cancer is a hypermutated cancer. In some embodiments, the invention includes a method of treating a cancer with a population of TILs, wherein the cancer is an enhanced hypermutated cancer. In some embodiments, the invention includes a method of treating a cancer with a population of TILs, wherein the cancer is an ultra-hypermutated cancer.

In some embodiments, the invention includes a method of treating a cancer with a population of TILs, wherein a patient is pre-treated with non-myeloablative chemotherapy prior to an infusion of TILs according to the present disclosure. In some embodiments, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In some embodiments, the non-myeloablative chemotherapy is cyclophosphamide 60 mg/kg/d for 2 days (days 27 and 26 prior to TIL infusion) and fludarabine 25 mg/m2/d for 3 days (days 27 to 25 prior to TIL infusion). In some embodiments, the non-myeloablative chemotherapy is cyclophosphamide 60 mg/kg/d for 2 days (days 27 and 26 prior to TIL infusion) followed by fludarabine 25 mg/m2/d for 3 days (days 25 to 23 prior to TIL infusion). In some embodiments, after non-myeloablative chemotherapy and TIL infusion (at day 0) according to the present disclosure, the patient receives an intravenous infusion of IL-2 intravenously at 720,000 IU/kg every 8 hours to physiologic tolerance.

Efficacy of the compounds and combinations of compounds described herein in treating, preventing and/or managing the indicated diseases or disorders can be tested using various models known in the art, which provide guidance for treatment of human disease. For example, models for determining efficacy of treatments for ovarian cancer are described, e.g., in Mullany, et al., Endocrinology 2012, 153, 1585-92; and Fong, et al., Ovarian Res. 2009, 2, 12. Models for determining efficacy of treatments for pancreatic cancer are described in Herreros-Villanueva, et al., World,/Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1. 32.

In some embodiments, IFN-gamma (IFN-γ) is indicative of treatment efficacy for hyperproliferative disorder treatment. In some embodiments, IFN-γ in the blood of subjects treated with TILs is indicative of active TILs. In some embodiments, a potency assay for IFN-γ production is employed. IFN-γ production is another measure of cytotoxic potential. IFN-γ production can be measured by determining the levels of the cytokine IFN-γ in the blood of a subject treated with TILs prepared by the methods of the present invention, including those as described for example in FIG. 1 (in particular, e.g., FIG. 1B). In some embodiments, the TILs obtained by the present method provide for increased IFN-γ in the blood of subjects treated with the TILs of the present method as compared subjects treated with TILs prepared using methods referred to as the Gen 3 process, as exemplified FIG. 1 (in particular, e.g., FIG. 1B) and throughout this application. In some embodiments, an increase in IFN-γ is indicative of treatment efficacy in a patient treated with the TILs produced by the methods of the present invention. In some embodiments, IFN-γ is increased one-fold, two-fold, three-fold, four-fold, or five-fold or more as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased one-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased two-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased three-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased four-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ secretion is increased five-fold as compared to an untreated patient and/or as compared to a patient treated with TILs prepared using other methods than those provide herein including for example, methods other than those embodied in FIG. 1 (in particular, e.g., FIG. 1B). In some embodiments, IFN-γ is measured using a Quantikine ELISA kit. In some embodiments, IFN-γ is measured using a Quantikine ELISA kit. In some embodiments, IFN-γ is measured in TILs ex vivo from a patient treated with the TILs produced by the methods of the present invention. In some embodiments, IFN-γ is measured in blood in a patient treated with the TILs produced by the methods of the present invention. In some embodiments, IFN-γ is measured in serum in a patient treated with the TILs produced by the methods of the present invention.

In some embodiments, the TILs prepared by the methods of the present invention, including those as described for example in FIG. 1 (in particular, e.g., FIG. 1B), exhibit increased polyclonality as compared to TILs produced by other methods, including those not exemplified in FIG. 1 (in particular, e.g., FIG. 1B), such as for example, methods referred to as process 1C methods. In some embodiments, significantly improved polyclonality and/or increased polyclonality is indicative of treatment efficacy and/or increased clinical efficacy for cancer treatment. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1 (in particular, e.g., FIG. 1B). In some embodiments, polyclonality is increased one-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased two-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased ten-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased 100-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased 500-fold as compared to an untreated patient and/or as compared to a patient treated with TILs prepared using other methods than those provide herein including for example, methods other than those embodied in FIG. 1 (in particular, e.g., FIG. 1B). In some embodiments, polyclonality is increased 1000-fold as compared to an untreated patient and/or as compared to a patient treated with TILs prepared using other methods than those provide herein including for example, methods other than those embodied in FIG. 1 (in particular, e.g., FIG. 1B).

2. Methods of Co-Administration

In some embodiments, the TILs produced as described herein, including for example TILs derived from a method described in Steps A through F of FIG. 1 (in particular, e.g., FIG. 1B), can be administered in combination with one or more immune checkpoint regulators, such as the antibodies described below. For example, antibodies that target PD-1 and which can be co-administered with the TILs of the present invention include, e.g., but are not limited to nivolumab (BMS-936558, Bristol-Myers Squibb; Opdivo®), pembrolizumab (lambrolizumab, MK03475 or MK-3475, Merck; Keytruda®), H12.1, PD1.3.1, SYM021, NAT 105, humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable antibodies suitable for use in co-administration methods with TILs produced according to Steps A through F as described herein are anti-PD-1 antibodies disclosed in U.S. Pat. No. 8,008,449, herein incorporated by reference. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in co-administration methods with TILs produced according to Steps A through F as described herein. For example, antibodies that target PD-L1 and are in clinical trials, include BMS-936559 (Bristol-Myers Squibb) and MPDL3280A (Genentech). Other suitable antibodies that target PD-L1 are disclosed in U.S. Pat. No. 7,943,743, herein incorporated by reference. It will be understood by one of ordinary skill that any antibody which binds to PD-1 or PD-L1, disrupts the PD-1/PD-L1 interaction, and stimulates an anti-tumor immune response, are suitable for use in co-administration methods with TILs produced according to Steps A through F as described herein. In some embodiments, the subject administered the combination of TILs produced according to Steps A through F is co administered with a and anti-PD-1 antibody when the patient has a cancer type that is refractory to administration of the anti-PD-1 antibody alone. In some embodiments, the patient is administered TILs in combination with and anti-PD-1 when the patient has refractory melanoma. In some embodiments, the patient is administered TILs in combination with and anti-PD-1 when the patient has non-small-cell lung carcinoma (NSCLC).

3. PD-1 and PD-L1 Inhibitors

In some embodiments, the TILs therapy provided to patients with cancer may include treatment with therapeutic populations of TILs alone or may include a combination treatment including TILs and one or more PD-1 and/or PD-L1 inhibitors.

Programmed death 1 (PD-1) is a 288-amino acid transmembrane immunocheckpoint receptor protein expressed by T cells, B cells, natural killer (NK) T cells, activated monocytes, and dendritic cells. PD-1, which is also known as CD279, belongs to the CD28 family, and in humans is encoded by the Pdcd1 gene on chromosome 2. PD-1 consists of one immunoglobulin (Ig) superfamily domain, a transmembrane region, and an intracellular domain containing an immunoreceptor tyrosine-based inhibitory motif (ITIM) and an immunoreceptor tyrosine-based switch motif (ITSM). PD-1 and its ligands (PD-L1 and PD-L2) are known to play a key role in immune tolerance, as described in Keir, et al., Annu. Rev. Immunol. 2008, 26, 677-704. PD-1 provides inhibitory signals that negatively regulate T cell immune responses. PD-L1 (also known as B7-H1 or CD274) and PD-L2 (also known as B7-DC or CD273) are expressed on tumor cells and stromal cells, which may be encountered by activated T cells expressing PD-1, leading to immunosuppression of the T cells. PD-L1 is a 290 amino acid transmembrane protein encoded by the Cd274 gene on human chromosome 9. Blocking the interaction between PD-1 and its ligands PD-L1 and PD-L2 by use of a PD-1 inhibitor, a PD-L1 inhibitor, and/or a PD-L2 inhibitor can overcome immune resistance, as demonstrated in recent clinical studies, such as that described in Topalian, et al., N. Eng. J. Med. 2012, 366, 2443-54. PD-L1 is expressed on many tumor cell lines, while PD-L2 is expressed is expressed mostly on dendritic cells and a few tumor lines. In addition to T cells (which inducibly express PD-1 after activation), PD-1 is also expressed on B cells, natural killer cells, macrophages, activated monocytes, and dendritic cells.

In some embodiments, the PD-1 inhibitor may be any PD-1 inhibitor or PD-1 blocker known in the art. In particular, it is one of the PD-1 inhibitors or blockers described in more detail in the following paragraphs. The terms “inhibitor,” “antagonist,” and “blocker” are used interchangeably herein in reference to PD-1 inhibitors. For avoidance of doubt, references herein to a PD-1 inhibitor that is an antibody may refer to a compound or antigen-binding fragments, variants, conjugates, or biosimilars thereof. For avoidance of doubt, references herein to a PD-1 inhibitor may also refer to a small molecule compound or a pharmaceutically acceptable salt, ester, solvate, hydrate, cocrystal, or prodrug thereof.

In some embodiments, the PD-1 inhibitor is an antibody (i.e., an anti-PD-1 antibody), a fragment thereof, including Fab fragments, or a single-chain variable fragment (scFv) thereof. In some embodiments the PD-1 inhibitor is a polyclonal antibody. In some embodiments, the PD-1 inhibitor is a monoclonal antibody. In some embodiments, the PD-1 inhibitor competes for binding with PD-1, and/or binds to an epitope on PD-1. In some embodiments, the antibody competes for binding with PD-1, and/or binds to an epitope on PD-1.

In some embodiments, the PD-1 inhibitor is one that binds human PD-1 with a K_{D} of about 100 pM or lower, binds human PD-1 with a K_{D} of about 90 pM or lower, binds human PD-1 with a KD of about 80 pM or lower, binds human PD-1 with a K_{D} of about 70 pM or lower, binds human PD-1 with a K_{D} of about 60 pM or lower, binds human PD-1 with a K_{D} of about 50 pM or lower, binds human PD-1 with a K_{D} of about 40 pM or lower, binds human PD-1 with a K_{D} of about 30 pM or lower, binds human PD-1 with a K_{D} of about 20 pM or lower, binds human PD-1 with a KD of about 10 pM or lower, or binds human PD-1 with a K_{D} of about 1 pM or lower.

In some embodiments, the PD-1 inhibitor is one that binds to human PD-1 with a kassoc of about 7.5×105 1/M·s or faster, binds to human PD-1 with a kassoc of about 7.5×105 1/M·s or faster, binds to human PD-1 with a kassoc of about 8×105 1/M·s or faster, binds to human PD-1 with a kassoc of about 8.5×105 1/M·s or faster, binds to human PD-1 with a kassoc of about 9×105 1/M·s or faster, binds to human PD-1 with a kassoc of about 9.5×105 1/M·s or faster, or binds to human PD-1 with a kassoc of about 1×106 1/M·s or faster.

In some embodiments, the PD-1 inhibitor is one that binds to human PD-1 with a kdissoc of about 2×10-5 1/s or slower, binds to human PD-1 with a kdissoc of about 2.1×10-5 1/s or slower, binds to human PD-1 with a kdissoc of about 2.2×10-5 1/s or slower, binds to human PD-1 with a kdissoc of about 2.3×10-5 1/s or slower, binds to human PD-1 with a kdissoc of about 2.4×10-5 1/s or slower, binds to human PD-1 with a kdissoc of about 2.5×10-5 1/s or slower, binds to human PD-1 with a kdissoc of about 2.6×10-5 1/s or slower or binds to human PD-1 with a kdissoc of about 2.7×10-5 1/s or slower, binds to human PD-1 with a kdissoc of about 2.8×10-5 1/s or slower, binds to human PD-1 with a kdissoc of about 2.9×10-5 1/s or slower, or binds to human PD-1 with a kdissoc of about 3×10-5 1/s or slower.

In some embodiments, the PD-1 inhibitor is one that blocks or inhibits binding of human PD-L1 or human PD-L2 to human PD-1 with an IC50 of about 10 nM or lower, blocks or inhibits binding of human PD-L1 or human PD-L2 to human PD-1 with an IC50 of about 9 nM or lower, blocks or inhibits binding of human PD-L1 or human PD-L2 to human PD-1 with an IC50 of about 8 nM or lower, blocks or inhibits binding of human PD-L1 or human PD-L2 to human PD-1 with an IC50 of about 7 nM or lower, blocks or inhibits binding of human PD-L1 or human PD-L2 to human PD-1 with an IC50 of about 6 nM or lower, blocks or inhibits binding of human PD-L1 or human PD-L2 to human PD-1 with an IC50 of about 5 nM or lower, blocks or inhibits binding of human PD-L1 or human PD-L2 to human PD-1 with an IC50 of about 4 nM or lower, blocks or inhibits binding of human PD-L1 or human PD-L2 to human PD-1 with an IC50 of about 3 nM or lower, blocks or inhibits binding of human PD-L1 or human PD-L2 to human PD-1 with an IC50 of about 2 nM or lower, or blocks or inhibits binding of human PD-L1 or human PD-L2 to human PD-1 with an IC50 of about 1 nM or lower.

In some embodiments, the PD-1 inhibitor is nivolumab (commercially available as OPDIVO from Bristol-Myers Squibb Co.), or biosimilars, antigen-binding fragments, conjugates, or variants thereof. Nivolumab is a fully human IgG4 antibody blocking the PD-1 receptor. In some embodiments, the anti-PD-1 antibody is an immunoglobulin G4 kappa, anti-(human CD274) antibody. Nivolumab is assigned Chemical Abstracts Service (CAS) registry number 946414-94-4 and is also known as 5C4, BMS-936558, MDX-1106, and ONO-4538. The preparation and properties of nivolumab are described in U.S. Pat. No. 8,008,449 and International Patent Publication No. WO 2006/121168, the disclosures of which are incorporated by reference herein. The clinical safety and efficacy of nivolumab in various forms of cancer has been described in Wang, et al., Cancer Immunol Res. 2014, 2, 846-56; Page, et al., Ann. Rev. Med., 2014, 65, 185-202; and Weber, et al., J. Clin. Oncology, 2013, 31, 4311-4318, the disclosures of which are incorporated by reference herein. The amino acid sequences of nivolumab are set forth in Table 19. Nivolumab has intra-heavy chain disulfide linkages at 22-96,140-196, 254-314, 360-418, 22″-96″, 140″-196″, 254″-314″, and 360″-418″; intra-light chain disulfide linkages at 23′-88′, 134′-194′, 23′″-88′″, and 134′″-194′″; inter-heavy-light chain disulfide linkages at 127-214′, 127″-214′″, inter-heavy-heavy chain disulfide linkages at 219-219″ and 222-222″; and N-glycosylation sites (H CH₂ 84.4) at 290, 290″.

In some embodiments, a PD-1 inhibitor comprises a heavy chain given by SEQ ID NO:463 and a light chain given by SEQ ID NO:464. In some embodiments, a PD-1 inhibitor comprises heavy and light chains having the sequences shown in SEQ ID NO:463 and SEQ ID NO:464, respectively, or antigen binding fragments, Fab fragments, single-chain variable fragments (scFv), variants, or conjugates thereof. In some embodiments, a PD-1 inhibitor comprises heavy and light chains that are each at least 99% identical to the sequences shown in SEQ ID NO:463 and SEQ ID NO:464, respectively. In some embodiments, a PD-1 inhibitor comprises heavy and light chains that are each at least 98% identical to the sequences shown in SEQ ID NO:463 and SEQ ID NO:464, respectively. In some embodiments, a PD-1 inhibitor comprises heavy and light chains that are each at least 97% identical to the sequences shown in SEQ ID NO:463 and SEQ ID NO:464, respectively. In some embodiments, a PD-1 inhibitor comprises heavy and light chains that are each at least 96% identical to the sequences shown in SEQ ID NO:463 and SEQ ID NO:464, respectively. In some embodiments, a PD-1 inhibitor comprises heavy and light chains that are each at least 95% identical to the sequences shown in SEQ ID NO:463 and SEQ ID NO:464, respectively.

In some embodiments, the PD-1 inhibitor comprises the heavy and light chain CDRs or variable regions (VRs) of nivolumab. In some embodiments, the PD-1 inhibitor heavy chain variable region (VH) comprises the sequence shown in SEQ ID NO:465, and the PD-1 inhibitor light chain variable region (VL) comprises the sequence shown in SEQ ID NO:466, and conservative amino acid substitutions thereof. In some embodiments, a PD-1 inhibitor comprises VH and VL regions that are each at least 99% identical to the sequences shown in SEQ ID NO:465 and SEQ ID NO:466, respectively. In some embodiments, a PD-1 inhibitor comprises VH and VL regions that are each at least 98% identical to the sequences shown in SEQ ID NO:465 and SEQ ID NO:466, respectively. In some embodiments, a PD-1 inhibitor comprises VH and VL regions that are each at least 97% identical to the sequences shown in SEQ ID NO:465 and SEQ ID NO:466, respectively. In some embodiments, a PD-1 inhibitor comprises VH and VL regions that are each at least 96% identical to the sequences shown in SEQ ID NO:465 and SEQ ID NO:466, respectively. In some embodiments, a PD-1 inhibitor comprises VH and VL regions that are each at least 95% identical to the sequences shown in SEQ ID NO:465 and SEQ ID NO:466, respectively.

In some embodiments, a PD-1 inhibitor comprises heavy chain CDR1, CDR2 and CDR3 domains having the sequences set forth in SEQ ID NO:467, SEQ ID NO:468, and SEQ ID NO:469, respectively, and conservative amino acid substitutions thereof, and light chain CDR1, CDR2 and CDR3 domains having the sequences set forth in SEQ ID NO:470, SEQ ID NO:471, and SEQ ID NO:472, respectively, and conservative amino acid substitutions thereof. In some embodiments, the antibody competes for binding with, and/or binds to the same epitope on PD-1 as any of the aforementioned antibodies.

In some embodiments, the PD-1 inhibitor is an anti-PD-1 biosimilar monoclonal antibody approved by drug regulatory authorities with reference to nivolumab. In some embodiments, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is nivolumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab.

TABLE 19
Amino acid sequences for PD-1 inhibitors related to nivolumab.

| Identifier | Sequence (One-Letter Amino Acid Symbols) | |
|---|---|---|
| SEQ ID NO: 463 | QVQLVESGGG VVCPGRSLRL DCKASGITFS NSGMHWVRQA PGKGLEWVAV IWYDGSKRYY | 60 |
| nivolumab | ADSVKGRFTI SRENSKNTLF IQMNSLRAED TAVYYCATND DYWGQGTLVT VSSASTKGPS | 120 |
| heavy chain | VFPLAPCSRS TSESTAALGC LVKDYFPEPV TVSWNSGALT SGVHTFPAVL QSSGLYSLSS | 180 |
| | VVTVPSSSLG TKTYTCNVDH KPSNTKVDKR VESKYGPPCP FCPAPEFLGG PSVFLFPPKP | 240 |
| | KDTLMISRTP EVTCVVVDVS QEDPEVQFNW YVDGVEVHNA KTKPREEQFN STYRVVSVLT | 300 |
| | VLHQDWLNGK EYKCKVSNKG LPSSIEKTIS KAKGQPREPQ VYTLPPSQEE MTKNQVSLTC | 360 |
| | LVKGFYPSDI AVEWESNGQP ENNYKTTPPV LDSDGSFFLY SRLTVDKSRW QEGNVFSCSV | 420 |
| | MHEALHNHYT QKSLSLSLGK | 440 |
| SEQ ID NO: 464 | EIVLTQSPAT LSISPGERAT LSCRASQSVS SYLAWYQQKP GQAPRLLIYD ASNRATGIPA | 60 |
| nivolumab | RFSGSGSGTD FTITISSLEP EDFAVYYCQQ SSNWPRTFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 465 | QVQLVESGGG VVCPGRSLRL DCKASGITFS NSGMHWVRQA FGKGLEWVAV IWYDGSKRYY | 60 |
| nivolumab | ADSVKGRFTI SRENSKNTLF IQMNSLRAED TAVYYCATND DYWGQGTLVT VSS | 113 |
| variable heavy | | |
| chain | | |
| SEQ ID NO: 466 | EIVLTQSPAT LSISIGERAT LSCRASQSVS SYLAWYQQKP GQAPRLLIYD ASNRATGIPA | 60 |
| nivolumab | RFSGSGSGTD FTITISSLEP EDFAVYYCQQ SSNWPRTFGQ GTKVEIK | 107 |
| variable light | | |
| chain | | |
| SEQ ID NO: 467 | NSGMH | 5 |
| nivolumab | | |
| heavy chain | | |
| CDR1 | | |
| SEQ ID NO: 468 | VIWYDGSKRY YADSVKG | 17 |
| nivolumab | | |
| heavy chain | | |
| CDR2 | | |
| SEQ ID NO: 469 | NDDY | 4 |
| nivolumab | | |
| heavy chain | | |
| CDR3 | | |
| SEQ ID NO: 470 | RASQSVSSYL A | 11 |
| nivolumab | | |
| light chain | | |
| CDR1 | | |
| SEQ ID NO: 471 | DASNRAT | 7 |
| nivolumab | | |
| light chain | | |
| CDR2 | | |
| SEQ ID NO: 472 | QQSSNWPRT | 9 |
| nivolumab | | |
| light chain | | |
| CDR3 | | |

In some embodiments, the PD-1 inhibitor is nivolumab or a biosimilar thereof, and the nivolumab is administered at a dose of about 0.5 mg/kg to about 10 mg/kg. In some embodiments, the PD-1 inhibitor is nivolumab or a biosimilar thereof, and the nivolumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the PD-1 inhibitor is nivolumab or a biosimilar thereof, and the nivolumab is administered at a dose of about 200 mg to about 500 mg. In some embodiments, the PD-1 inhibitor is nivolumab or a biosimilar thereof, and the nivolumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the PD-1 inhibitor is nivolumab or a biosimilar thereof, and the nivolumab is administered every 2 weeks, every 3 weeks, every 4 weeks, every 5 weeks, or every 6 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat unresectable or metastatic melanoma. In some embodiments, the nivolumab is administered to treat unresectable or metastatic melanoma and is administered at about 240 mg every 2 weeks. In some embodiments, the nivolumab is administered to treat unresectable or metastatic melanoma and is administered at about 480 mg every 4 weeks. In some embodiments, the nivolumab is administered to treat unresectable or metastatic melanoma and is administered at about 1 mg/kg followed by ipilimumab 3 mg/kg on the same day every 3 weeks for 4 doses, then 240 mg every 2 weeks or 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered for the adjuvant treatment of melanoma. In some embodiments, the nivolumab is administered for the adjuvant treatment of melanoma at about 240 mg every 2 weeks. In some embodiments, the nivolumab is administered for the adjuvant treatment of melanoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat metastatic non-small cell lung cancer. In some embodiments, the nivolumab is administered to treat metastatic non-small cell lung cancer at about 3 mg/kg every 2 weeks along with ipilimumab at about 1 mg/kg every 6 weeks. In some embodiments, the nivolumab is administered to treat metastatic non-small cell lung cancer at about 360 mg every 3 weeks with ipilimumab 1 mg/kg every 6 weeks and 2 cycles of platinum-doublet chemotherapy. In some embodiments, the nivolumab is administered to treat metastatic non-small cell lung cancer at about 240 mg every 2 weeks or 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat small cell lung cancer. In some embodiments, the nivolumab is administered to treat small cell lung cancer at about 240 mg every 2 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat malignant pleural mesothelioma at about 360 mg every 3 weeks with ipilimumab 1 mg/kg every 6 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat advanced renal cell carcinoma. In some embodiments, the nivolumab is administered to treat advanced renal cell carcinoma at about 240 mg every 2 weeks. In some embodiments, the nivolumab is administered to treat advanced renal cell carcinoma at about 480 mg every 4 weeks. In some embodiments, the nivolumab is administered to treat advanced renal cell carcinoma at about 3 mg/kg followed by ipilimumab at about 1 mg/kg on the same day every 3 weeks for 4 doses, then 240 mg every 2 weeks. In some embodiments, the nivolumab is administered to treat advanced renal cell carcinoma at about 3 mg/kg followed by ipilimumab at about 1 mg/kg on the same day every 3 weeks for 4 doses, then 240 mg every 2 weeks 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat classical Hodgkin lymphoma. In some embodiments, the nivolumab is administered to treat classical Hodgkin lymphoma at about 240 mg every 2 weeks. In some embodiments, the nivolumab is administered to treat classical Hodgkin lymphoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat Recurrent or metastatic squamous cell carcinoma of the head and neck. In some embodiments, the nivolumab is administered to treat recurrent or metastatic squamous cell carcinoma of the head and neck at about 240 mg every 2 weeks. In some embodiments, the nivolumab is administered to treat recurrent or metastatic squamous cell carcinoma of the head and neck at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat locally advanced or metastatic urothelial carcinoma at about 240 mg every 2 weeks. In some embodiments, the nivolumab is administered to treat locally advanced or metastatic urothelial carcinoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat microsatellite instability-high (MSI-H) or mismatch repair deficient (dMMR) metastatic colorectal cancer. In some embodiments, the nivolumab is administered to treat Microsatellite instability-high (MSI-H) or mismatch repair deficient (dMMR) metastatic colorectal cancer in adult and pediatric patients. In some embodiments, the nivolumab is administered to treat microsatellite instability-high (MSI-H) or mismatch repair deficient (dMMR) metastatic colorectal cancer in adult and pediatric patients ≥40 kg at about 240 mg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat microsatellite instability-high (MSI-H) or mismatch repair deficient (dMMR) metastatic colorectal cancer in pediatric patients <40 kg at about 3 mg/kg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 3 mg/kg followed by ipilimumab 1 mg/kg on the same day every 3 weeks for 4 doses, then 240 mg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 3 mg/kg followed by ipilimumab 1 mg/kg on the same day every 3 weeks for 4 doses, then 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat hepatocellular carcinoma. In some embodiments, the nivolumab is administered to treat hepatocellular carcinoma at about 240 mg every 2 weeks. In some embodiments, the nivolumab is administered to treat hepatocellular carcinoma at about 480 mg every 4 weeks. In some embodiments, the nivolumab is administered to treat hepatocellular carcinoma at about 1 mg/kg followed by ipilimumab 3 mg/kg on the same day every 3 weeks for 4 doses, then 240 mg every 2 weeks. In some embodiments, the nivolumab is administered to treat hepatocellular carcinoma at about 1 mg/kg followed by ipilimumab 3 mg/kg on the same day every 3 weeks for 4 doses, then 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat esophageal squamous cell carcinoma. In some embodiments, the nivolumab is administered to treat esophageal squamous cell carcinoma at about 240 mg every 2 weeks. In some embodiments, the nivolumab is administered to treat esophageal squamous cell carcinoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In other embodiments, the PD-1 inhibitor comprises pembrolizumab (commercially available as KEYTRUDA from Merck & Co., Inc., Kenilworth, NJ, USA), or antigen-binding fragments, conjugates, or variants thereof. Pembrolizumab is assigned CAS registry number 1374853-91-4 and is also known as lambrolizumab, MK-3475, and SCH-900475. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline (H10-S>P)]γ4 heavy chain (134-218′)-disulfide with humanized mouse monoclonal κ light chain dimer (226-226″: 229-229″)-bisdisulfide. The properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2, the disclosures of which are incorporated herein by reference. The clinical safety and efficacy of pembrolizumab in various forms of cancer is described in Fuerst, Oncology Times, 2014, 36, 35-36; Robert, et al., Lancet, 2014, 384, 1109-17; and Thomas, et al., Exp. Opin. Biol. Ther., 2014; 14, 1061-1064. The amino acid sequences of pembrolizumab are set forth in Table 20. Pembrolizumab includes the following disulfide bridges: 22-96, 22″-96″, 23′-92′, 23′″-92′″, 134-218′, 134″-218″, 138′-198′, 138′″-198′″, 147-203, 147″-203″, 226-226″, 229-229″, 261-321, 261″-321″, 367-425, and 367″-425″, and the following glycosylation sites (N): Asn-297 and Asn-297″. Pembrolizumab is an IgG4/kappa isotype with a stabilizing S228P mutation in the Fc region; insertion of this mutation in the IgG4 hinge region prevents the formation of half molecules typically observed for IgG4 antibodies. Pembrolizumab is heterogeneously glycosylated at Asn297 within the Fc domain of each heavy chain, yielding a molecular weight of approximately 149 kDa for the intact antibody. The dominant glycoform of pembrolizumab is the fucosylated agalacto diantennary glycan form (GOF).

In some embodiments, a PD-1 inhibitor comprises a heavy chain given by SEQ ID NO:473 and a light chain given by SEQ ID NO:474. In some embodiments, a PD-1 inhibitor comprises heavy and light chains having the sequences shown in SEQ ID NO:473 and SEQ ID NO:474, respectively, or antigen binding fragments, Fab fragments, single-chain variable fragments (scFv), variants, or conjugates thereof. In some embodiments, a PD-1 inhibitor comprises heavy and light chains that are each at least 99% identical to the sequences shown in SEQ ID NO:473 and SEQ ID NO:474, respectively. In some embodiments, a PD-1 inhibitor comprises heavy and light chains that are each at least 98% identical to the sequences shown in SEQ ID NO:473 and SEQ ID NO:474, respectively. In some embodiments, a PD-1 inhibitor comprises heavy and light chains that are each at least 97% identical to the sequences shown in SEQ ID NO:473 and SEQ ID NO:474, respectively. In some embodiments, a PD-1 inhibitor comprises heavy and light chains that are each at least 96% identical to the sequences shown in SEQ ID NO:473 and SEQ ID NO:474, respectively. In some embodiments, a PD-1 inhibitor comprises heavy and light chains that are each at least 95% identical to the sequences shown in SEQ ID NO:473 and SEQ ID NO:474, respectively.

In some embodiments, the PD-1 inhibitor comprises the heavy and light chain CDRs or variable regions (VRs) of pembrolizumab. In some embodiments, the PD-1 inhibitor heavy chain variable region (VH) comprises the sequence shown in SEQ ID NO:475, and the PD-1 inhibitor light chain variable region (VL) comprises the sequence shown in SEQ ID NO:476, and conservative amino acid substitutions thereof. In some embodiments, a PD-1 inhibitor comprises VH and VL regions that are each at least 99% identical to the sequences shown in SEQ ID NO:475 and SEQ ID NO:476, respectively. In some embodiments, a PD-1 inhibitor comprises VH and VL regions that are each at least 98% identical to the sequences shown in SEQ ID NO:475 and SEQ ID NO:476, respectively. In some embodiments, a PD-1 inhibitor comprises VH and VL regions that are each at least 97% identical to the sequences shown in SEQ ID NO:475 and SEQ ID NO:476, respectively. In some embodiments, a PD-1 inhibitor comprises VH and VL regions that are each at least 96% identical to the sequences shown in SEQ ID NO:475 and SEQ ID NO:476, respectively. In some embodiments, a PD-1 inhibitor comprises VH and VL regions that are each at least 95% identical to the sequences shown in SEQ ID NO:475 and SEQ ID NO:476, respectively.

In some embodiments, a PD-1 inhibitor comprises the heavy chain CDR1, CDR2 and CDR3 domains having the sequences set forth in SEQ ID NO:477, SEQ ID NO:478, and SEQ ID NO:479, respectively, and conservative amino acid substitutions thereof, and light chain CDR1, CDR2 and CDR3 domains having the sequences set forth in SEQ ID NO:480, SEQ ID NO:481, and SEQ ID NO:482, respectively, and conservative amino acid substitutions thereof. In some embodiments, the antibody competes for binding with, and/or binds to the same epitope on PD-1 as any of the aforementioned antibodies.

In some embodiments, the PD-1 inhibitor is an anti-PD-1 biosimilar monoclonal antibody approved by drug regulatory authorities with reference to pembrolizumab. In some embodiments, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pembrolizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pembrolizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab.

TABLE 20
Amino acid sequences for PD-1 inhibitors related to pembrolizumab.

| Identifier | Sequence (One-Letter Amino Acid Symbols) | |
|---|---|---|
| SEQ ID NO: 473 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| heavy chain | ASTKGPSVFP LAPCSRSTSE STAALGCLVK DYFPEPVTVS WNSGALTSGV HTFPAVLQSS | 180 |
| | GLYSLSSVVT VPSSSLGTKT YTCNVDHKPS NTKVDKRVES KYGPPCPPCP APEFLGGPSV | 240 |
| | FLFPPKPKDT LMISRTPEVT CVVVDVSQED PEVQFNWYVD GVEVHNAKTK PREEQFNSTY | 300 |
| | RVVSVLTVLH QDWLNGKEYK CKVSNKGLPS SIEKTISKAK GQPREPQVYT LPPSQEEMTK | 360 |
| | NQVSLTCLVK GFYPSDIAVE WESNGQPENN YKITPPVLDS DGSFFLYSRL TVDKSRWQEG | 420 |
| | NVFSCSVMHE ALHNHYTQKS LSLSLGK | 447 |
| SEQ ID NO: 474 | EIVLTQSPAT LSLIFGERAT LSCRASKGVS TSGYSYLHWY QQKPGQAPRL LIYLASYLES | 60 |
| pembroli zumab | GVPARFSGSG SGTDFTLTIS SLEPEDFAVY YCQHSRDLPL TFGGGTKVEI KRTVAAPSVF | 120 |
| light chain | IFPPSDEQLK SGTASVVCLL NNFYPREAKV QWKVDNALQS GNSQESVTEQ DSKDSTYSLS | 180 |
| | STLTLSKADY EKHKVYACEV THQGLSSPVT KSFNRGEC | 218 |
| SEQ ID NO: 475 | QVQLVQSGVE VKKPGASVKV SCKASGYTFT NYYMYWVRQA PGQGLEWMGG INPSNGGTNF | 60 |
| pembroli zumab | NEKFKNRVTL TTDSSTTTAY MELKSLQFDD TAVYYCARRD YRFDMGFDYW GQGTTVTVSS | 120 |
| variable heavy | | |
| chain | | |
| SEQ ID NO: 476 | EIVLTQSPAT LSISPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI K | 111 |
| variable light | | |
| chain | | |
| SEQ ID NO: 477 | NYYMY | 5 |
| pembrolizumab | | |
| heavy chain | | |
| CDR1 | | |
| SEQ ID NO: 478 | GINPSNGGTN FNEKFK | 16 |
| pembroli zumab | | |
| heavy chain | | |
| CDR2 | | |
| SEQ ID NO: 479 | RDYRFDMGFD Y | 11 |
| pembroli zumab | | |
| heavy chain | | |
| CDR3 | | |
| SEQ ID NO: 480 | RASKGVSTSG YSYLH | 15 |
| pembroli zumab | | |
| light chain | | |
| CDR1 | | |
| SEQ ID NO: 481 | LASYLES | 7 |
| pembroli zumab | | |
| light chain | | |
| CDR2 | | |
| SEQ ID NO: 482 | QHSRDLPLT | 9 |
| pembroli zumab | | |
| light chain | | |
| CDR3 | | |

In some embodiments, the PD-1 inhibitor is pembrolizumab or a biosimilar thereof, and the pembrolizumab is administered at a dose of about 0.5 mg/kg to about 10 mg/kg. In some embodiments, the PD-1 inhibitor is pembrolizumab or a biosimilar thereof, and the pembrolizumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is pembrolizumab or a biosimilar thereof, wherein the pembrolizumab is administered at a dose of about 200 mg to about 500 mg. In some embodiments, the PD-1 inhibitor is pembrolizumab or a biosimilar thereof, and the nivolumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is pembrolizumab or a biosimilar thereof, wherein the pembrolizumab is administered every 2 weeks, every 3 weeks, every 4 weeks, every 5 weeks, or every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat melanoma. In some embodiments, the pembrolizumab is administered to treat melanoma at about 200 mg every 3 weeks. In some embodiments, the pembrolizumab is administered to treat melanoma at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat NSCLC. In some embodiments, the pembrolizumab is administered to treat NSCLC at about 200 mg every 3 weeks. In some embodiments, the pembrolizumab is administered to treat NSCLC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat small cell lung cancer (SCLC). In some embodiments, the pembrolizumab is administered to treat SCLC at about 200 mg every 3 weeks. In some embodiments, the pembrolizumab is administered to treat SCLC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat head and neck squamous cell cancer (HNSCC). In some embodiments, the pembrolizumab is administered to treat HNSCC at about 200 mg every 3 weeks. In some embodiments, the pembrolizumab is administered to treat HNSCCat about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Classical Hodgkin Lymphoma (cHL) or Primary Mediastinal Large B-Cell Lymphoma (PMBCL) at about 200 mg every 3 weeks. In some embodiments, the pembrolizumab is administered to treat Classical Hodgkin Lymphoma (cHL) or Primary Mediastinal Large B-Cell Lymphoma (PMBCL) at about 400 mg every 6 weeks for adults. In some embodiments, the pembrolizumab is administered to treat Classical Hodgkin Lymphoma (cHL) or Primary Mediastinal Large B-Cell Lymphoma (PMBCL) at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat urothelial carcinoma at about 200 mg every 3 weeks. In some embodiments, the pembrolizumab is administered to treat urothelial carcinoma at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat microsatellite instability-high (MSI-H) or mismatch repair deficient (dMMR) cancer at about 200 mg every 3 weeks. In some embodiments, the pembrolizumab is administered to treat MSI-H or dMMR cancer at about 400 mg every 6 weeks for adults. In some embodiments, the pembrolizumab is administered to treat MSI-H or dMMR cancer at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient). In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat microsatellite instability-high (MSI-H) or mismatch repair deficient colorectal cancer (dMMR CRC at about 200 mg every 3 weeks. In some embodiments, the pembrolizumab is administered to treat MSI-H or dMMR CRC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat gastric cancer at about 200 mg every 3 weeks. In some embodiments, the pembrolizumab is administered to treat gastric cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Esophageal Cancer at about 200 mg every 3 weeks. In some embodiments, the pembrolizumab is administered to treat Esophageal Cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat cervical cancer at about 200 mg every 3 weeks. In some embodiments, the pembrolizumab is administered to treat cervical cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat hepatocellular carcinoma (HCC) at about 200 mg every 3 weeks. In some embodiments, the pembrolizumab is administered to treat HCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Merkel cell carcinoma (MCC) at about 200 mg every 3 weeks for adults. In some embodiments, the pembrolizumab is administered to treat MCC at about 400 mg every 6 weeks for adults. In some embodiments, the pembrolizumab is administered to treat MCC at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient). In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat renal cell carcinoma (RCC) at about 200 mg every 3 weeks. In some embodiments, the pembrolizumab is administered to treat RCC at about 400 mg every 6 weeks with axitinib 5 mg orally twice daily. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat endometrial carcinoma at about 200 mg every 3 weeks. In some embodiments, the pembrolizumab is administered to treat Endometrial Carcinoma at about 400 mg every 6 weeks with lenvatinib 20 mg orally once daily for tumors that are not MSI-H or dMMR. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat tumor mutational burden-high (TMB-H) Cancer at about 200 mg every 3 weeks for adults. In some embodiments, the pembrolizumab is administered to treat TMB-H Cancer at about 400 mg every 6 weeks for adults. In some embodiments, the pembrolizumab is administered to treat TMB-H Cancer at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat cutaneous squamous cell carcinoma (cSCC) at about 200 mg every 3 weeks. In some embodiments, the pembrolizumab is administered to treat cSCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat triple-negative breast cancer (TNBC) at about 200 mg every 3 weeks. In some embodiments, the pembrolizumab is administered to treat TNBC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, if the patient or subject is an adult, i.e., treatment of adult indications, and additional dosing regimen of 400 mg every 6 weeks can be employed. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is a commercially-available anti-PD-1 monoclonal antibody, such as anti-m-PD-1 clones J43 (Cat #BE0033-2) and RMP1-14 (Cat #BE0146) (Bio X Cell, Inc., West Lebanon, NH, USA). A number of commercially-available anti-PD-1 antibodies are known to one of ordinary skill in the art.

In some embodiments, the PD-1 inhibitor is an antibody disclosed in U.S. Pat. No. 8,354,509 or U.S. Patent Application Publication Nos. 2010/0266617 A1, 2013/0108651 A1, 2013/0109843 A2, the disclosures of which are incorporated by reference herein. In some embodiments, the PD-1 inhibitor is an anti-PD-1 antibody described in U.S. Pat. Nos. 8,287,856; 8,580,247, and 8,168,757 and U.S. Patent Application Publication Nos. 2009/0028857 A1, 2010/0285013 A1, 2013/0022600 A1, and 2011/0008369 A1, the teachings of which are hereby incorporated by reference. In other embodiments, the PD-1 inhibitor is an anti-PD-1 antibody disclosed in U.S. Pat. No. 8,735,553 B1, the disclosure of which is incorporated herein by reference. In some embodiments, the PD-1 inhibitor is pidilizumab, also known as CT-011, which is described in U.S. Pat. No. 8,686,119, the disclosure of which is incorporated by reference herein.

In some embodiments, the PD-1 inhibitor may be a small molecule or a peptide, or a peptide derivative, such as those described in U.S. Pat. Nos. 8,907,053; 9,096,642; and 9,044,442 and U.S. Patent Application Publication No. US 2015/0087581; 1,2,4-oxadiazole compounds and derivatives such as those described in U.S. Patent Application Publication No. 2015/0073024; cyclic peptidomimetic compounds and derivatives such as those described in U.S. Patent Application Publication No. US 2015/0073042; cyclic compounds and derivatives such as those described in U.S. Patent Application Publication No. US 2015/0125491; 1,3,4-oxadiazole and 1,3,4-thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. US 2014/0294898; the disclosures of each of which are hereby incorporated by reference in their entireties.

In some embodiments, the PD-L1 or PD-L2 inhibitor may be any PD-L1 or PD-L2 inhibitor, antagonist, or blocker known in the art. In particular, it is one of the PD-L1 or PD-L2 inhibitors, antagonist, or blockers described in more detail in the following paragraphs. The terms “inhibitor,” “antagonist,” and “blocker” are used interchangeably herein in reference to PD-L1 and PD-L2 inhibitors. For avoidance of doubt, references herein to a PD-L1 or PD-L2 inhibitor that is an antibody may refer to a compound or antigen-binding fragments, variants, conjugates, or biosimilars thereof. For avoidance of doubt, references herein to a PD-L1 or PD-L2 inhibitor may refer to a compound or a pharmaceutically acceptable salt, ester, solvate, hydrate, cocrystal, or prodrug thereof.

In some embodiments, the compositions, processes and methods described herein include a PD-L1 or PD-L2 inhibitor. In some embodiments, the PD-L1 or PD-L2 inhibitor is a small molecule. In some embodiments, the PD-L1 or PD-L2 inhibitor is an antibody (i.e., an anti-PD-1 antibody), a fragment thereof, including Fab fragments, or a single-chain variable fragment (scFv) thereof. In some embodiments the PD-L1 or PD-L2 inhibitor is a polyclonal antibody. In some embodiments, the PD-L1 or PD-L2 inhibitor is a monoclonal antibody. In some embodiments, the PD-L1 or PD-L2 inhibitor competes for binding with PD-L1 or PD-L2, and/or binds to an epitope on PD-L1 or PD-L2. In some embodiments, the antibody competes for binding with PD-L1 or PD-L2, and/or binds to an epitope on PD-L1 or PD-L2.

In some embodiments, the PD-L1 inhibitors provided herein are selective for PD-L1, in that the compounds bind or interact with PD-L1 at substantially lower concentrations than they bind or interact with other receptors, including the PD-L2 receptor. In certain embodiments, the compounds bind to the PD-L1 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L2 receptor.

In some embodiments, the PD-L2 inhibitors provided herein are selective for PD-L2, in that the compounds bind or interact with PD-L2 at substantially lower concentrations than they bind or interact with other receptors, including the PD-L1 receptor. In certain embodiments, the compounds bind to the PD-L2 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L1 receptor.

Without being bound by any theory, it is believed that tumor cells express PD-L1, and that T cells express PD-1. However, PD-L1 expression by tumor cells is not required for efficacy of PD-1 or PD-L1 inhibitors or blockers. In some embodiments, the tumor cells express PD-L1. In other embodiments, the tumor cells do not express PD-L1. In some embodiments, the methods can include a combination of a PD-1 and a PD-L1 antibody, such as those described herein, in combination with a TIL. The administration of a combination of a PD-1 and a PD-L1 antibody and a TIL may be simultaneous or sequential.

In some embodiments, the PD-L1 and/or PD-L2 inhibitor is one that binds human PD-L1 and/or PD-L2 with a K_{D} of about 100 pM or lower, binds human PD-L1 and/or PD-L2 with a KD of about 90 pM or lower, binds human PD-L1 and/or PD-L2 with a K_{D} of about 80 pM or lower, binds human PD-L1 and/or PD-L2 with a K_{D} of about 70 pM or lower, binds human PD-L1 and/or PD-L2 with a K_{D} of about 60 pM or lower, a KD of about 50 pM or lower, binds human PD-L1 and/or PD-L2 with a K_{D} of about 40 pM or lower, or binds human PD-L1 and/or PD-L2 with a K_{D} of about 30 pM or lower,

In some embodiments, the PD-L1 and/or PD-L2 inhibitor is one that binds to human PD-L1 and/or PD-L2 with a kassoc of about 7.5×105 1/M·s or faster, binds to human PD-L1 and/or PD-L2 with a kassoc of about 8×105 1/M·s or faster, binds to human PD-L1 and/or PD-L2 with a kassoc of about 8.5×105 1/M·s or faster, binds to human PD-L1 and/or PD-L2 with a kassoc of about 9×105 1/M·s or faster, binds to human PD-L1 and/or PD-L2 with a kassoc of about 9.5×105 1/M·s and/or faster, or binds to human PD-L1 and/or PD-L2 with a kassoc of about 1×106 1/M s or faster.

In some embodiments, the PD-L1 and/or PD-L2 inhibitor is one that binds to human PD-L1 or PD-L2 with a kdissoc of about 2×10-5 1/s or slower, binds to human PD-1 with a kdissoc of about 2.1×10-5 1/s or slower, binds to human PD-1 with a kdissoc of about 2.2×10-5 1/s or slower, binds to human PD-1 with a kdissoc of about 2.3×10-5 1/s or slower, binds to human PD-1 with a kdissoc of about 14×10-5 1/s or slower, binds to human PD-1 with a kdissoc of about 2.5×10-5 1/s or slower, binds to human PD-1 with a kdissoc of about 2.6×10-5 1/s or slower, binds to human PD-L1 or PD-L2 with a kdissoc of about 2.7×10-5 1/s or slower, or binds to human PD-L1 or PD-L2 with a kdissoc of about 3×10-5 1/s or slower.

In some embodiments, the PD-L1 and/or PD-L2 inhibitor is one that blocks or inhibits binding of human PD-L1 or human PD-L2 to human PD-1 with an IC50 of about 10 nM or lower; blocks or inhibits binding of human PD-L1 or human PD-L2 to human PD-1 with an IC50 of about 9 nM or lower; blocks or inhibits binding of human PD-L1 or human PD-L2 to human PD-1 with an IC50 of about 8 nM or lower; blocks or inhibits binding of human PD-L1 or human PD-L2 to human PD-1 with an IC50 of about 7 nM or lower; blocks or inhibits binding of human PD-L1 or human PD-L2 to human PD-1 with an IC50 of about 6 nM or lower; blocks or inhibits binding of human PD-L1 or human PD-L2 to human PD-1 with an IC50 of about 5 nM or lower; blocks or inhibits binding of human PD-L1 or human PD-L2 to human PD-1 with an IC50 of about 4 nM or lower; blocks or inhibits binding of human PD-L1 or human PD-L2 to human PD-1 with an IC50 of about 3 nM or lower; blocks or inhibits binding of human PD-L1 or human PD-L2 to human PD-1 with an IC50 of about 2 nM or lower; or blocks human PD-1, or blocks binding of human PD-L1 or human PD-L2 to human PD-1 with an IC50 of about 1 nM or lower.

In some embodiments, the PD-L1 inhibitor is durvalumab, also known as MEDI4736 (which is commercially available from Medimmune, LLC, Gaithersburg, Md., a subsidiary of AstraZeneca plc.), or antigen-binding fragments, conjugates, or variants thereof. In some embodiments, the PD-L1 inhibitor is an antibody disclosed in U.S. Pat. No. 8,779,108 or U.S. Patent Application Publication No. 2013/0034559, the disclosures of which are incorporated by reference herein. The clinical efficacy of durvalumab has been described in Page, et al., Ann. Rev. Med., 2014, 65, 185-202; Brahmer, et al., J. Clin. Oncol. 2014, 32, 5s (supplement, abstract 8021); and McDermott, et al., Cancer Treatment Rev., 2014, 40, 1056-64. The preparation and properties of durvalumab are described in U.S. Pat. No. 8,779,108, the disclosure of which is incorporated by reference herein. The amino acid sequences of durvalumab are set forth in Table 21. The durvalumab monoclonal antibody includes disulfide linkages at 22-96, 22″-96″, 23′-89′, 23′″-89″′, 135′-195′, 135′″-195′″, 148-204, 148″-204″, 215′-224, 215′″-224″, 230-230″, 233-233″, 265-325, 265″-325″, 371-429, and 371″-429′; and N-glycosylation sites at Asn-301 and Asn-301″.

In some embodiments, a PD-L1 inhibitor comprises a heavy chain given by SEQ ID NO:483 and a light chain given by SEQ ID NO:484. In some embodiments, a PD-L1 inhibitor comprises heavy and light chains having the sequences shown in SEQ ID NO:483 and SEQ ID NO:484, respectively, or antigen binding fragments, Fab fragments, single-chain variable fragments (scFv), variants, or conjugates thereof. In some embodiments, a PD-L1 inhibitor comprises heavy and light chains that are each at least 99% identical to the sequences shown in SEQ ID NO:483 and SEQ ID NO:484, respectively. In some embodiments, a PD-L1 inhibitor comprises heavy and light chains that are each at least 98% identical to the sequences shown in SEQ ID NO:483 and SEQ ID NO:484, respectively. In some embodiments, a PD-L1 inhibitor comprises heavy and light chains that are each at least 97% identical to the sequences shown in SEQ ID NO:483 and SEQ ID NO:484, respectively. In some embodiments, a PD-L1 inhibitor comprises heavy and light chains that are each at least 96% identical to the sequences shown in SEQ ID NO:483 and SEQ ID NO:484, respectively. In some embodiments, a PD-L1 inhibitor comprises heavy and light chains that are each at least 95% identical to the sequences shown in SEQ ID NO:483 and SEQ ID NO:484, respectively.

In some embodiments, the PD-L1 inhibitor comprises the heavy and light chain CDRs or variable regions (VRs) of durvalumab. In some embodiments, the PD-L1 inhibitor heavy chain variable region (VH) comprises the sequence shown in SEQ ID NO:485, and the PD-L1 inhibitor light chain variable region (VL) comprises the sequence shown in SEQ ID NO:486, and conservative amino acid substitutions thereof. In some embodiments, a PD-L1 inhibitor comprises VH and VL regions that are each at least 99% identical to the sequences shown in SEQ ID NO:485 and SEQ ID NO:486, respectively. In some embodiments, a PD-L1 inhibitor comprises VH and VL regions that are each at least 98% identical to the sequences shown in SEQ ID NO:485 and SEQ ID NO:486, respectively. In some embodiments, a PD-L1 inhibitor comprises VH and VL regions that are each at least 97% identical to the sequences shown in SEQ ID NO:485 and SEQ ID NO:486, respectively. In some embodiments, a PD-L1 inhibitor comprises VH and VL regions that are each at least 96% identical to the sequences shown in SEQ ID NO:485 and SEQ ID NO:486, respectively. In some embodiments, a PD-L1 inhibitor comprises VH and VL regions that are each at least 95% identical to the sequences shown in SEQ ID NO:485 and SEQ ID NO:486, respectively.

In some embodiments, a PD-L1 inhibitor comprises heavy chain CDR1, CDR2 and CDR3 domains having the sequences set forth in SEQ ID NO:487, SEQ ID NO:488, and SEQ ID NO:489, respectively, and conservative amino acid substitutions thereof, and light chain CDR1, CDR2 and CDR3 domains having the sequences set forth in SEQ ID NO:490, SEQ ID NO:491, and SEQ ID NO:492, respectively, and conservative amino acid substitutions thereof. In some embodiments, the antibody competes for binding with, and/or binds to the same epitope on PD-L1 as any of the aforementioned antibodies.

In some embodiments, the PD-L1 inhibitor is an anti-PD-L1 biosimilar monoclonal antibody approved by drug regulatory authorities with reference to durvalumab. In some embodiments, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is durvalumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is durvalumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab.

TABLE 21
Amino acid sequences for PD-L1 inhibitors related to durvalumab.

| Identifier | Sequence (One-Letter Amino Acid Symbols) | |
|---|---|---|
| SEQ ID NO: 483 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY IQMNSLRAED TAVYYCAREG GWFGELAFDY WGQGTLVTVS | 120 |
| heavy chain | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPEFEG | 240 |
| | GPSVFLFPPK PKDTLMISRT PEVTCVVVDV SHEDPEVKEN WYVDGVEVHN AKTKPREEQY | 300 |
| | NSTYRVVSVL TVLHQDWING KEYKCKVSNK ALPASIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 484 | EVQLVESGGG LVQPGGSLRL SCAASGFTFS RYWMSWVRQA PGKGLEWVAN EIVLTQSPGT | 60 |
| durvalumab | LSLSPGERAT LSCRASQRVS SSYLAWYQQK PGQAPRLLIY DASSRATGIP DRFSGSGSGT | 120 |
| light chain | DFTLTISRLE PEDFAVYYCQ QYGSLPWTFG QGTKVEIKRT VAAPSVFIFP PSDEQLKSGT | 180 |
| | ASVVCLLNNF YPREAKVQWK VDNALQSGNS QESVTEQDSK DSTYSLSSTL TLSKADYEKH | 240 |
| | KVYACEVTHQ GLSSPVTKSF NRGEC | 265 |
| SEQ ID NO: 485 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY IQMNSLRAED TAVYYCAREG GWFGELAFDY WGQGTLVTVS | 120 |
| variable | S | 121 |
| heavy chain | | |
| SEQ ID NO: 486 | EIVLTQSPGT LSLSPGERAT LSCRASQRVS SSYLAWYQQK PGQAPRLLIY DASSRATGIP | 60 |
| durvalumab | DRFSGSGSGT DFTLTISRLE PEDFAVYYCQ QYGSLPWTFG QGTKVEIK | 108 |
| variable | | |
| light chain | | |
| SEQ ID NO: 487 | RYWMS | 5 |
| durvalumab | | |
| heavy chain | | |
| CDR1 | | |
| SEQ ID NO: 488 | NIKQDGSEKY YVDSVKG | 17 |
| durvalumab | | |
| heavy chain | | |
| CDR2 | | |
| SEQ ID NO: 489 | EGGWFGELAF DY | 12 |
| durvalumab | | |
| heavy chain | | |
| CDR3 | | |
| SEQ ID NO: 490 | RASQRVSSSY LA | 12 |
| durvalumab | | |
| light chain | | |
| CDR1 | | |
| SEQ ID NO: 491 | DASSRAT | 7 |
| durvalumab | | |
| light chain | | |
| CDR2 | | |
| SEQ ID NO: 492 | QQYGSLPWT | 9 |
| durvalumab | | |
| light chain | | |
| CDR3 | | |

In some embodiments, the PD-L1 inhibitor is avelumab, also known as MSB0010718C (commercially available from Merck KGaA/EMD Serono), or antigen-binding fragments, conjugates, or variants thereof. The preparation and properties of avelumab are described in U.S. Patent Application Publication No. US 2014/0341917 A1, the disclosure of which is specifically incorporated by reference herein. The amino acid sequences of avelumab are set forth in Table 22. Avelumab has intra-heavy chain disulfide linkages (C23-C104) at 22-96, 147-203, 264-324, 370-428, 22″-96″, 147″-203″, 264″-324″, and 370″-428″; intra-light chain disulfide linkages (C23-C104) at 22′-90′, 138′-197′, 22′″-90′″, and 138′″-197″; intra-heavy-light chain disulfide linkages (h 5-CL 126) at 223-215′ and 223″-215′″; intra-heavy-heavy chain disulfide linkages (h 11, h 14) at 229-229″ and 232-232″; N-glycosylation sites (H CH₂ N84.4) at 300, 300″; fucosylated complex bi-antennary CHO-type glycans; and H CHS K2 C-terminal lysine clipping at 450 and 450′.

In some embodiments, a PD-L1 inhibitor comprises a heavy chain given by SEQ ID NO:493 and a light chain given by SEQ ID NO:494. In some embodiments, a PD-L1 inhibitor comprises heavy and light chains having the sequences shown in SEQ ID NO:493 and SEQ ID NO:494, respectively, or antigen binding fragments, Fab fragments, single-chain variable fragments (scFv), variants, or conjugates thereof. In some embodiments, a PD-L1 inhibitor comprises heavy and light chains that are each at least 99% identical to the sequences shown in SEQ ID NO:493 and SEQ ID NO:494, respectively. In some embodiments, a PD-L1 inhibitor comprises heavy and light chains that are each at least 98% identical to the sequences shown in SEQ ID NO:493 and SEQ ID NO:494, respectively. In some embodiments, a PD-L1 inhibitor comprises heavy and light chains that are each at least 97% identical to the sequences shown in SEQ ID NO:493 and SEQ ID NO:494, respectively. In some embodiments, a PD-L1 inhibitor comprises heavy and light chains that are each at least 96% identical to the sequences shown in SEQ ID NO:493 and SEQ ID NO:494, respectively. In some embodiments, a PD-L1 inhibitor comprises heavy and light chains that are each at least 95% identical to the sequences shown in SEQ ID NO:493 and SEQ ID NO:494, respectively.

In some embodiments, the PD-L1 inhibitor comprises the heavy and light chain CDRs or variable regions (VRs) of avelumab. In some embodiments, the PD-L1 inhibitor heavy chain variable region (VH) comprises the sequence shown in SEQ ID NO:495, and the PD-L1 inhibitor light chain variable region (VL) comprises the sequence shown in SEQ ID NO:496, and conservative amino acid substitutions thereof. In some embodiments, a PD-L1 inhibitor comprises VH and VL regions that are each at least 99% identical to the sequences shown in SEQ ID NO:495 and SEQ ID NO:496, respectively. In some embodiments, a PD-L1 inhibitor comprises VH and VL regions that are each at least 98% identical to the sequences shown in SEQ ID NO:496 and SEQ ID NO:496, respectively. In some embodiments, a PD-L1 inhibitor comprises VH and VL regions that are each at least 97% identical to the sequences shown in SEQ ID NO:495 and SEQ ID NO:496, respectively. In some embodiments, a PD-L1 inhibitor comprises VH and VL regions that are each at least 96% identical to the sequences shown in SEQ ID NO:495 and SEQ ID NO:496, respectively. In some embodiments, a PD-L1 inhibitor comprises VH and VL regions that are each at least 95% identical to the sequences shown in SEQ ID NO:495 and SEQ ID NO:496, respectively.

In some embodiments, a PD-L1 inhibitor comprises heavy chain CDR1, CDR2 and CDR3 domains having the sequences set forth in SEQ ID NO:497, SEQ ID NO:498, and SEQ ID NO:499, respectively, and conservative amino acid substitutions thereof, and light chain CDR1, CDR2 and CDR3 domains having the sequences set forth in SEQ ID NO:500, SEQ ID NO:501, and SEQ ID NO:502, respectively, and conservative amino acid substitutions thereof. In some embodiments, the antibody competes for binding with, and/or binds to the same epitope on PD-L1 as any of the aforementioned antibodies.

In some embodiments, the PD-L1 inhibitor is an anti-PD-L1 biosimilar monoclonal antibody approved by drug regulatory authorities with reference to avelumab. In some embodiments, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is avelumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is avelumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab.

TABLE 22
Amino acid sequences for PD-L1 inhibitors related to avelumab.

| Identifier | Sequence (One-Letter Amino Acid Symbols) | |
|---|---|---|
| SEQ ID NO: 493 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY IQMNSLRAED TAVYYCARIK LGTVTTVDYW GQGTLVTVSS | 120 |
| heavy chain | ASTKGPSVFP LAPSSKSTSG GTAALGCLVK DYFPEPVTVS WNSGALTSEV HTFPAVLQSS | 180 |
| | GLYSLSSVVT VPSSSLGTQT YICNVNHKPS NTKVDKKVEP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREFQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 494 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL GQPKANPTVT | 120 |
| light chain | LFPPSSEELQ ANKATLVCLI SDFYPGAVTV AWKADGSPVK AGVETTKPSK QSNNKYAASS | 180 |
| | YLSLTPEQWK SHRSYSCQVT HEGSTVEKTV APTECS | 216 |
| SEQ ID NO: 495 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY IQMNSLRAED TAVYYCARIK LGTVTTVDYW GQGTLVTVSS | 120 |
| variable | | |
| heavy chain | | |
| SEQ ID NO: 496 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL | 110 |
| variable | | |
| light chain | | |
| SEQ ID NO: 497 | SYIMM | 5 |
| avelumab | | |
| heavy chain | | |
| CDR1 | | |
| SEQ ID NO: 498 | STYPSGGITF YADTVKG | 17 |
| avelumab | | |
| heavy chain | | |
| CDR2 | | |
| SEQ ID NO: 499 | IKLGTVTTVD Y | 11 |
| avelumab | | |
| heavy chain | | |
| CDR3 | | |
| SEQ ID NO: 500 | TGTSSDVGGY NYVS | 14 |
| avelumab | | |
| light chain | | |
| CDR1 | | |
| SEQ ID NO: 501 | DVSNRPS | 7 |
| avelumab | | |
| light chain | | |
| CDR2 | | |
| SEQ ID NO: 502 | SSYTSSSTRV | 10 |
| avelumab | | |
| light chain | | |
| CDR3 | | |

In some embodiments, the PD-L1 inhibitor is atezolizumab, also known as MPDL3280A or RG7446 (commercially available as TECENTRIQ from Genentech, Inc., a subsidiary of Roche Holding AG, Basel, Switzerland), or antigen-binding fragments, conjugates, or variants thereof. In some embodiments, the PD-L1 inhibitor is an antibody disclosed in U.S. Pat. No. 8,217,149, the disclosure of which is specifically incorporated by reference herein. In some embodiments, the PD-L1 inhibitor is an antibody disclosed in U.S. Patent Application Publication Nos. 2010/0203056 A1, 2013/0045200 A1, 2013/0045201 A1, 2013/0045202 A1, or 2014/0065135 A1, the disclosures of which are specifically incorporated by reference herein. The preparation and properties of atezolizumab are described in U.S. Pat. No. 8,217,149, the disclosure of which is incorporated by reference herein. The amino acid sequences of atezolizumab are set forth in Table 23. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′, 134′-194′, 23′″-88′″, and 134″′-194′; intra-heavy-light chain disulfide linkages (h 5-CL 126) at 221-214′ and 221″-214″; intra-heavy-heavy chain disulfide linkages (h 11, h 14) at 227-227″ and 230-230″; and N-glycosylation sites (H CH₂ N84.4>A) at 298 and 298′.

In some embodiments, a PD-L1 inhibitor comprises a heavy chain given by SEQ ID NO:503 and a light chain given by SEQ ID NO:504. In some embodiments, a PD-L1 inhibitor comprises heavy and light chains having the sequences shown in SEQ ID NO:503 and SEQ ID NO:504, respectively, or antigen binding fragments, Fab fragments, single-chain variable fragments (scFv), variants, or conjugates thereof. In some embodiments, a PD-L1 inhibitor comprises heavy and light chains that are each at least 99% identical to the sequences shown in SEQ ID NO:503 and SEQ ID NO:504, respectively. In some embodiments, a PD-L1 inhibitor comprises heavy and light chains that are each at least 98% identical to the sequences shown in SEQ ID NO:503 and SEQ ID NO:504, respectively. In some embodiments, a PD-L1 inhibitor comprises heavy and light chains that are each at least 97% identical to the sequences shown in SEQ ID NO:503 and SEQ ID NO:504, respectively. In some embodiments, a PD-L1 inhibitor comprises heavy and light chains that are each at least 96% identical to the sequences shown in SEQ ID NO:503 and SEQ ID NO:504, respectively. In some embodiments, a PD-L1 inhibitor comprises heavy and light chains that are each at least 95% identical to the sequences shown in SEQ ID NO:503 and SEQ ID NO:504, respectively.

In some embodiments, the PD-L1 inhibitor comprises the heavy and light chain CDRs or variable regions (VRs) of atezolizumab. In some embodiments, the PD-L1 inhibitor heavy chain variable region (VH) comprises the sequence shown in SEQ ID NO:505, and the PD-L1 inhibitor light chain variable region (VL) comprises the sequence shown in SEQ ID NO:506, and conservative amino acid substitutions thereof. In some embodiments, a PD-L1 inhibitor comprises VH and VL regions that are each at least 99% identical to the sequences shown in SEQ ID NO:505 and SEQ ID NO:506, respectively. In some embodiments, a PD-L1 inhibitor comprises VH and VL regions that are each at least 98% identical to the sequences shown in SEQ ID NO:505 and SEQ ID NO:506, respectively. In some embodiments, a PD-L1 inhibitor comprises VH and VL regions that are each at least 97% identical to the sequences shown in SEQ ID NO:505 and SEQ ID NO:506, respectively. In some embodiments, a PD-L1 inhibitor comprises VH and VL regions that are each at least 96% identical to the sequences shown in SEQ ID NO:505 and SEQ ID NO:506, respectively. In some embodiments, a PD-L1 inhibitor comprises VH and VL regions that are each at least 95% identical to the sequences shown in SEQ ID NO:505 and SEQ ID NO:506, respectively.

In some embodiments, a PD-L1 inhibitor comprises heavy chain CDR1, CDR2 and CDR3 domains having the sequences set forth in SEQ ID NO:507, SEQ ID NO:508, and SEQ ID NO:509, respectively, and conservative amino acid substitutions thereof, and light chain CDR1, CDR2 and CDR3 domains having the sequences set forth in SEQ ID NO:510, SEQ ID NO:511, and SEQ ID NO:512, respectively, and conservative amino acid substitutions thereof. In some embodiments, the antibody competes for binding with, and/or binds to the same epitope on PD-L1 as any of the aforementioned antibodies.

In some embodiments, the anti-PD-L1 antibody is an anti-PD-L1 biosimilar monoclonal antibody approved by drug regulatory authorities with reference to atezolizumab. In some embodiments, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is atezolizumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is atezolizumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab.

TABLE 23
Amino acid sequences for PD-L1 inhibitors related to atezolizumab.

| Identifier | Sequence (One-Letter Amino Acid Symbols) | |
|---|---|---|
| SEQ ID NO: 503 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY IQMNSLRAED TAVYYCARRH WPGGFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKKVEPKS CDKTHTCPFC PAPELLGGPS | 240 |
| | VFLFPPKPKD TLMISRTPEV TCVVVDVSHE DPEVKFNWYV DGVEVHNAKT KPREEQYAST | 300 |
| | YRVVSVLTVL HQDWLNGKEY KCKVSNKALP APIEKTISKA KGQPREFQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 504 | DIQMTQSPSS LSASVGDRVT ITCRASQDVS TAVAWYQQKP GKAPKLLIYS ASFLYSGVPS | 60 |
| atezolizumab | RFSGSGSGTD FILTISSLQP EDFATYYCQQ YLYHPATFGQ GTKVEIKRIV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 505 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY IQMNSLRAED TAVYYCARRH WPGGFDYWGQ GTLVTVSA | 118 |
| variable | | |
| heavy chain | | |
| SEQ ID NO: 506 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKR | 108 |
| variable | | |
| light chain | | |
| SEQ ID NO: 507 | GFTFSDSWIH | 10 |
| atezolizumab | | |
| heavy chain | | |
| CDR1 | | |
| SEQ ID NO: 508 | AWISPYGGST YYADSVKG | 18 |
| atezolizumab | | |
| heavy chain | | |
| CDR2 | | |
| SEQ ID NO: 509 | RHWPGGFDY | 9 |
| atezolizumab | | |
| heavy chain | | |
| CDR3 | | |
| SEQ ID NO: 510 | RASQDVSTAV A | 11 |
| atezolizumab | | |
| light chain | | |
| CDR1 | | |
| SEQ ID NO: 511 | SASFLYS | 7 |
| atezolizumab | | |
| light chain | | |
| CDR2 | | |
| SEQ ID NO: 512 | QQYLYHPAT | 9 |
| atezolizumab | | |
| light chain | | |
| CDR3 | | |

In some embodiments, PD-L1 inhibitors include those antibodies described in U.S. Patent Application Publication No. US 2014/0341917 A1, the disclosure of which is incorporated by reference herein. In other embodiments, antibodies that compete with any of these antibodies for binding to PD-L1 are also included. In some embodiments, the anti-PD-L1 antibody is MDX-1105, also known as BMS-935559, which is disclosed in U.S. Pat. No. 7,943,743, the disclosures of which are incorporated by reference herein. In some embodiments, the anti-PD-L1 antibody is selected from the anti-PD-L1 antibodies disclosed in U.S. Pat. No. 7,943,743, which are incorporated by reference herein.

In some embodiments, the PD-L1 inhibitor is a commercially-available monoclonal antibody, such as INVIVOMAB anti-m-PD-L1 clone 10F.9G2 (Catalog #BE0101, Bio X Cell, Inc., West Lebanon, NH, USA). In some embodiments, the anti-PD-L1 antibody is a commercially-available monoclonal antibody, such as AFFYMETRIX EBIOSCIENCE (MIH1). A number of commercially-available anti-PD-L1 antibodies are known to one of ordinary skill in the art.

In some embodiments, the PD-L2 inhibitor is a commercially-available monoclonal antibody, such as BIOLEGEND 24F.10C12 Mouse IgG2a, κ isotype (catalog #329602 Biolegend, Inc., San Diego, Calif.), SIGMA anti-PD-L2 antibody (catalog #SAB3500395, Sigma-Aldrich Co., St. Louis, Mo.), or other commercially-available anti-PD-L2 antibodies known to one of ordinary skill in the art.

4. CTLA-4 Inhibitors

In some embodiments, the TIL therapy provided to patients with cancer may include treatment with therapeutic populations of TILs alone or may include a combination treatment including TILs and one or more CTLA-4 inhibitors.

Cytotoxic T lymphocyte antigen 4 (CTLA-4) is a member of the immunoglobulin superfamily and is expressed on the surface of helper T cells. CTLA-4 is a negative regulator of CD28-dependent T cell activation and acts as a checkpoint for adaptive immune responses. Similar to the T cell costimulatory protein CD28, the CTLA-4 binding antigen presents CD80 and CD86 on the cells. CTLA-4 delivers a suppressor signal to T cells, while CD28 delivers a stimulus signal. Human antibodies against human CTLA-4 have been described as immunostimulatory modulators in many disease conditions, such as treating or preventing viral and bacterial infections and for treating cancer (WO 01/14424 and WO 00/37504). Various preclinical studies have shown that CTLA-4 blockade by CTLA-4 inhibitors such as monoclonal antibodies enhances host immune responses against immunogenic tumors and can even rule out established tumors. A number of fully human anti-human CTLA-4 monoclonal antibodies (mAbs) have been studied in clinical trials for the treatment of various types of solid tumors, including, but limited to, ipilimumab (MDX-010) and tremelimumab (CP-675,206).

In some embodiments, a CTLA-4 inhibitor may be any CTLA-4 inhibitor or CTLA-4 blocker known in the art. In particular, it is one of the CTLA-4 inhibitors or blockers described in more detail in the following paragraphs. The terms “inhibitor,” “antagonist,” and “blocker” are used interchangeably herein in reference to CTLA-4 inhibitors. For avoidance of doubt, references herein to a CTLA-4 inhibitor that is an antibody may refer to a compound or antigen-binding fragments, variants, conjugates, or biosimilars thereof. For avoidance of doubt, references herein to a CTLA-4 inhibitor may also refer to a small molecule compound or a pharmaceutically acceptable salt, ester, solvate, hydrate, cocrystal, or prodrug thereof.

Suitable CTLA-4 inhibitors for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1, the disclosures of each of which are incorporated herein by reference. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014, the disclosures of each of which are incorporated herein by reference. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22 (145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281, the disclosures of each of which are incorporated herein by reference.

Additional CTLA-4 inhibitors include, but are not limited to, the following: any inhibitor that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD8₀; CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other CTLA-4 inhibitors.

In some embodiments a CTLA-4 inhibitor binds to CTLA-4 with a Kd of about 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, e.g., between 10⁻¹³ M and 10⁻¹⁶ M, or within any range having any two of the afore-mentioned values as endpoints. In some embodiments a CTLA-4 inhibitor binds to CTLA-4 with a Kd of no more than 10-fold that of ipilimumab, when compared using the same assay. In some embodiments a CTLA-4 inhibitor binds to CTLA-4 with a Kd of about the same as, or less (e.g., up to 10-fold lower, or up to 100-fold lower) than that of ipilimumab, when compared using the same assay. In some embodiments, the IC50 values for inhibition by a CTLA-4 inhibitor of CTLA-4 binding to CD80 or CD86 is no more than 10-fold greater than that of ipilimumab-mediated inhibition of CTLA-4 binding to CD80 or CD86, respectively, when compared using the same assay. In some embodiments, the IC50 values for inhibition by a CTLA-4 inhibitor of CTLA-4 binding to CD80 or CD86 is about the same or less (e.g., up to 10-fold lower, or up to 100-fold lower) than that of ipilimumab-mediated inhibition of CTLA-4 binding to CD80 or CD86, respectively, when compared using the same assay.

In some embodiments a CTLA-4 inhibitor is used in an amount sufficient to inhibit expression and/or decrease biological activity of CTLA-4 by at least 20%, 30%, 40%, 50%, 60%, 70%, 80%, 90%, 95%, or 100% relative to a suitable control, e.g., between 50% and 75%, 75% and 90%, or 90% and 100%. In some embodiments a CTLA-4 pathway inhibitor is used in an amount sufficient to decrease the biological activity of CTLA-4 by reducing binding of CTLA-4 to CD80, CD86, or both by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. A suitable control in the context of assessing or quantifying the effect of an agent of interest is typically a comparable biological system (e.g., cells or a subject) that has not been exposed to or treated with the agent of interest, e.g., CTLA-4 pathway inhibitor (or has been exposed to or treated with a negligible amount). In some embodiments a biological system may serve as its own control (e.g., the biological system may be assessed before exposure to or treatment with the agent and compared with the state after exposure or treatment has started or finished. In some embodiments a historical control may be used.

In some embodiments, the CTLA-4 inhibitor is ipilimumab (commercially available as Yervoy from Bristol-Myers Squibb Co.), or biosimilars, antigen-binding fragments, conjugates, or variants thereof. As is known in the art, ipilimumab refers to an anti-CTLA-4 antibody, a fully human IgG 1κ antibody derived from a transgenic mouse with human genes encoding heavy and light chains to generate a functional human repertoire, is there. Ipilimumab can also be referred to by its CAS Registry Number 477202-00-9, and in PCT Publication Number WO 01/14424, which is incorporated herein by reference in its entirety for all purposes. It is disclosed as antibody 10DI. Specifically, ipilimumab contains a light chain variable region and a heavy chain variable region (having a light chain variable region comprising SEQ ID NO: 516 and having a heavy chain variable region comprising SEQ ID NO: 515). Represents a human monoclonal antibody or its antigen binding site that specifically binds to CTLA-4. A pharmaceutical composition of ipilimumab includes all pharmaceutically acceptable compositions containing ipilimumab and one or more diluents, vehicles and/or excipients. An example of a pharmaceutical composition containing ipilimumab is described in PCT Publication No. WO 2007/67959. Ipilimumab can be administered intravenously (IV).

In some embodiments, a CTLA-4 inhibitor comprises a heavy chain given by SEQ ID NO:513 and a light chain given by SEQ ID NO:514. In some embodiments, a CTLA-4 inhibitor comprises heavy and light chains having the sequences shown in SEQ ID NO:513 and SEQ ID NO:514, respectively, or antigen binding fragments, Fab fragments, single-chain variable fragments (scFv), variants, or conjugates thereof. In some embodiments, a CTLA-4 inhibitor comprises heavy and light chains that are each at least 99% identical to the sequences shown in SEQ ID NO:513 and SEQ ID NO:514, respectively. In some embodiments, a CTLA-4 inhibitor comprises heavy and light chains that are each at least 98% identical to the sequences shown in SEQ ID NO:513 and SEQ ID NO:514, respectively. In some embodiments, a CTLA-4 inhibitor comprises heavy and light chains that are each at least 97% identical to the sequences shown in SEQ ID NO:513 and SEQ ID NO:514, respectively. In some embodiments, a CTLA-4 inhibitor comprises heavy and light chains that are each at least 96% identical to the sequences shown in SEQ ID NO:513 and SEQ ID NO:514, respectively. In some embodiments, a CTLA-4 inhibitor comprises heavy and light chains that are each at least 95% identical to the sequences shown in SEQ ID NO:513 and SEQ ID NO:514, respectively.

In some embodiments, the CTLA-4 inhibitor comprises the heavy and light chain CDRs or variable regions (VRs) of ipilimumab. In some embodiments, the CTLA-4 inhibitor heavy chain variable region (VH) comprises the sequence shown in SEQ ID NO:515, and the CTLA-4 inhibitor light chain variable region (VL) comprises the sequence shown in SEQ ID NO:516, and conservative amino acid substitutions thereof. In some embodiments, a CTLA-4 inhibitor comprises V_{H} and V_{L} regions that are each at least 99% identical to the sequences shown in SEQ ID NO:515 and SEQ ID NO:516, respectively. In some embodiments, a CTLA-4 inhibitor comprises V_{H} and V_{L} regions that are each at least 98% identical to the sequences shown in SEQ ID NO:515 and SEQ ID NO:516, respectively. In some embodiments, a CTLA-4 inhibitor comprises V_{H} and V_{L} regions that are each at least 97% identical to the sequences shown in SEQ ID NO:515 and SEQ ID NO:516, respectively. In some embodiments, a CTLA-4 inhibitor comprises V_{H} and V_{L} regions that are each at least 96% identical to the sequences shown in SEQ ID NO:515 and SEQ ID NO:516, respectively. In some embodiments, a CTLA-4 inhibitor comprises V_{H} and V_{L} regions that are each at least 95% identical to the sequences shown in SEQ ID NO:515 and SEQ ID NO:516, respectively.

In some embodiments, a CTLA-4 inhibitor comprises the heavy chain CDR1, CDR2 and CDR3 domains having the sequences set forth in SEQ ID NO:517, SEQ ID NO:518, and SEQ ID NO:519, respectively, and conservative amino acid substitutions thereof, and light chain CDR1, CDR2 and CDR3 domains having the sequences set forth in SEQ ID NO:520, SEQ ID NO:521, and SEQ ID NO:522, respectively, and conservative amino acid substitutions thereof. In some embodiments, the antibody competes for binding with, and/or binds to the same epitope on CTLA-4 as any of the aforementioned antibodies.

In some embodiments, the CTLA-4 inhibitor is a CTLA-4 biosimilar monoclonal antibody approved by drug regulatory authorities with reference to ipilimumab. In some embodiments, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is ipilimumab. In some embodiments, the one or more post-translational modifications are selected from one or more of: glycosylation, oxidation, deamidation, and truncation. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is ipilimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab.

TABLE 24
Amino acid sequences for ipilimumab.

| Identifier | Sequence (One-Letter Amino Acid Symbols) | |
|---|---|---|
| SEQ ID NO: 513 | QVQLVESGGG VVQPGRSLRL SCAASGFTFS SYTMHWVRQA PGKGLEWVTF ISYDGNNKYY | 1 |
| ipilimumab | ADSVKGRFTI SRDNSKNTLY IQMNSLRAED TAIYYCARTG WLGPFDYWGQ GTLVTVSSAS | 61 |
| heavy chain | TKGPSVFPLA PSSKSTSGGT AALGCLVKDY FPEPVTVSWN SGALTSGVHT FPAVLQSSGL | 121 |
| | YSLSSVVTVP SSSLGTQTYI CNVNHKPSNT KVDKRVEPKS CDKTH | 181 |
| SEQ ID NO: 514 | EIVLTQSPGT LSLSPGERAT LSCRASQSVG SSYLAWYQQK PGQAPRLLIY GAFSRATGIP | 1 |
| ipilimumab | DRFSGSGSGT DFTLTISRLE PEDFAVYYCQ QYGSSPWIFG QGTKVEIKRT VAAPSVFIFP | 61 |
| light chain | PSDEQLKSGT ASVVCLLNNF YPREAKVQWK VDNALQSGNS QESVTEQDSK DSTYSLSSTL | 121 |
| | TLSKADYEKH KVYACEVTHQ GLSSPVTKSF NRGEC | 181 |
| SEQ ID NO: 515 | QVQLVESGGG VVQPGRSLRL SCAASGFTFS SYTMHWVRQA PGKGLEWVTF ISYDGNNKYY | 1 |
| ipilimumab | ADSVKGRFTI SRDNSKNTLY IQMNSLRAED TAIYYCARTG WLGPFDYWGQ GTLVTVSS | 61 |
| variable heavy | | |
| chain | | |
| SEQ ID NO: 516 | EIVLTQSPGT LSLSPGERAT LSCRASQSVG SSYLAWYQQK PGQAPRLLIY GAFSRATGIP | 1 |
| ipilimumab | DRFSGSGSGT DFTLTISRLE PEDFAVYYCQ QYGSSPWTFG QGTKVEIK | 61 |
| variable light | | |
| chain | | |
| SEQ ID NO: 517 | GFTFSSYT | 8 |
| ipilimumab | | |
| heavy chain | | |
| CDR1 | | |
| SEQ ID NO: 518 | TFISYDGNNK | 10 |
| ipilimumab | | |
| heavy chain | | |
| CDR2 | | |
| SEQ ID NO: 519 | ARTGWLGPFD Y | 11 |
| ipilimumab | | |
| heavy chain | | |
| CDR3 | | |
| SEQ ID NO: 520 | QSVGSSY | 7 |
| ipilimumab | | |
| light chain | | |
| CDR1 | | |
| SEQ ID NO: 521 | GAF | 3 |
| ipilimumab | | |
| light chain | | |
| CDR2 | | |
| SEQ ID NO: 522 | QQYGSSPWT | 9 |
| ipilimumab | | |
| light chain | | |
| CDR3 | | |

In some embodiments, the CTLA-4 inhibitor is ipilimumab or a biosimilar thereof, and the ipilimumab is administered at a dose of about 0.5 mg/kg to about 10 mg/kg. In some embodiments, the CTLA-4 inhibitor is ipilimumab or a biosimilar thereof, and the ipilimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1.2, or 3 weeks pre-resection (i.e., prior to obtaining the tumor sample from the subject or patient).

In some embodiments, the CTLA-4 inhibitor is ipilimumab or a biosimilar thereof, and the ipilimumab is administered at a dose of about 200 mg to about 500 mg. In some embodiments, the CTLA-4 inhibitor is ipilimumab or a biosimilar thereof, and the ipilimumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the CTLA-4 inhibitor is ipilimumab or a biosimilar thereof, and the ipilimumab is administered every 2 weeks, every 3 weeks, every 4 weeks, every 5 weeks, or every 6 weeks. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat unresectable or metastatic melanoma. In some embodiments, the ipilimumab is administered to treat Unresectable or Metastatic Melanoma at about mg/kg every 3 weeks for a maximum of 4 doses. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered for the adjuvant treatment of melanoma. In some embodiments, the ipilimumab is administered to for the adjuvant treatment of melanoma at about 10 mg/kg every 3 weeks for 4 doses, followed by 10 mg/kg every 12 weeks for up to 3 years. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat advanced renal cell carcinoma. In some embodiments, the ipilimumab is administered to treat advanced renal cell carcinoma at about 1 mg/kg immediately following nivolumab 3 mg/kg on the same day, every 3 weeks for 4 doses. In some embodiments, after completing 4 doses of the combination, nivolumab can be administered as a single agent according to standard dosing regimens for advanced renal cell carcinoma and/or renal cell carcinoma. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat microsatellite instability-high (MSI-H) or mismatch repair deficient (dMMR) metastatic colorectal cancer. In some embodiments, the ipilimumab is administered to treat microsatellite instability-high (MSI-H) or mismatch repair deficient (dMMR) metastatic colorectal cancer at about 1 mg/kg intravenously over 30 minutes immediately following nivolumab 3 mg/kg intravenously over 30 minutes on the same day, every 3 weeks for 4 doses. In some embodiments, after completing 4 doses of the combination, administer nivolumab as a single agent as recommended according to standard dosing regimens for microsatellite instability-high (MSI-H) or mismatch repair deficient (dMMR) metastatic colorectal cancer. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat hepatocellular carcinoma. In some embodiments, the ipilimumab is administered to treat hepatocellular carcinoma at about 3 mg/kg intravenously over 30 minutes immediately following nivolumab 1 mg/kg intravenously over 30 minutes on the same day, every 3 weeks for 4 doses. In some embodiments, after completion 4 doses of the combination, administer nivolumab as a single agent according to standard dosing regimens for hepatocellular carcinoma. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat metastatic non-small cell lung cancer. In some embodiments, the ipilimumab is administered to treat metastatic non-small cell lung cancer at about 1 mg/kg every 6 weeks with nivolumab 3 mg/kg every 2 weeks. In some embodiments, the ipilimumab is administered to treat metastatic non-small cell lung cancer at about 1 mg/kg every 6 weeks with nivolumab 360 mg every 3 weeks and 2 cycles of platinum-doublet chemotherapy. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat malignant pleural mesothelioma. In some embodiments, the ipilimumab is administered to treat malignant pleural mesothelioma at about 1 mg/kg every 6 weeks with nivolumab 360 mg every 3 weeks. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

Tremelimumab (also known as CP-675,206) is a fully human IgG2 monoclonal antibody and has the CAS number 745013-59-6. Tremelimumab is disclosed as antibody 11.2.1 in U.S. Pat. No. 6,682,736 (incorporated herein by reference). The amino acid sequences of the heavy chain and light chain of tremelimumab are set forth in SEQ IND NOs:xx and xx, respectively. Tremelimumab has been investigated in clinical trials for the treatment of various tumors, including melanoma and breast cancer; in which Tremelimumab was administered intravenously either as single dose or multiple doses every 4 or 12 weeks at the dose range of 0.01 and 15 mg/kg. In the regimens provided by the present invention, tremelimumab is administered locally, particularly intradermally or subcutaneously. The effective amount of tremelimumab administered intradermally or subcutaneously is typically in the range of 5-200 mg/dose per person. In some embodiments, the effective amount of tremelimumab is in the range of 10-150 mg/dose per person per dose. In some particular embodiments, the effective amount of tremelimumab is about 10, 25, 37.5, 40, 50, 75, 100, 125, 150, 175, or 200 mg/dose per person.

In some embodiments, a CTLA-4 inhibitor comprises a heavy chain given by SEQ ID NO:523 and a light chain given by SEQ ID NO:524. In some embodiments, a CTLA-4 inhibitor comprises heavy and light chains having the sequences shown in SEQ ID NO:523 and SEQ ID NO:524, respectively, or antigen binding fragments, Fab fragments, single-chain variable fragments (scFv), variants, or conjugates thereof. In some embodiments, a CTLA-4 inhibitor comprises heavy and light chains that are each at least 99% identical to the sequences shown in SEQ ID NO:523 and SEQ ID NO:524, respectively. In some embodiments, a CTLA-4 inhibitor comprises heavy and light chains that are each at least 98% identical to the sequences shown in SEQ ID NO:523 and SEQ ID NO:524, respectively. In some embodiments, a CTLA-4 inhibitor comprises heavy and light chains that are each at least 97% identical to the sequences shown in SEQ ID NO:523 and SEQ ID NO:524, respectively. In some embodiments, a CTLA-4 inhibitor comprises heavy and light chains that are each at least 96% identical to the sequences shown in SEQ ID NO:523 and SEQ ID NO:524, respectively. In some embodiments, a CTLA-4 inhibitor comprises heavy and light chains that are each at least 95% identical to the sequences shown in SEQ ID NO:523 and SEQ ID NO:524, respectively.

In some embodiments, the CTLA-4 inhibitor comprises the heavy and light chain CDRs or variable regions (VRs) of tremelimumab. In some embodiments, the CTLA-4 inhibitor heavy chain variable region (V_{H}) comprises the sequence shown in SEQ ID NO:525, and the CTLA-4 inhibitor light chain variable region (V_{L}) comprises the sequence shown in SEQ ID NO:526, and conservative amino acid substitutions thereof. In some embodiments, a CTLA-4 inhibitor comprises V_{H} and V_{L} regions that are each at least 99% identical to the sequences shown in SEQ ID NO:525 and SEQ ID NO:526, respectively. In some embodiments, a CTLA-4 inhibitor comprises V_{H} and V_{L} regions that are each at least 98% identical to the sequences shown in SEQ ID NO:525 and SEQ ID NO:526, respectively. In some embodiments, a CTLA-4 inhibitor comprises V_{H} and V_{L} regions that are each at least 97% identical to the sequences shown in SEQ ID NO:525 and SEQ ID NO:526, respectively. In some embodiments, a CTLA-4 inhibitor comprises V_{H} and V_{L} regions that are each at least 96% identical to the sequences shown in SEQ ID NO: 525 and SEQ ID NO:526, respectively. In some embodiments, a CTLA-4 inhibitor comprises V_{H} and V_{L} regions that are each at least 95% identical to the sequences shown in SEQ ID NO: 525 and SEQ ID NO:526, respectively.

In some embodiments, a CTLA-4 inhibitor comprises the heavy chain CDR1, CDR2 and CDR3 domains having the sequences set forth in SEQ ID NO:527, SEQ ID NO:528, and SEQ ID NO:529, respectively, and conservative amino acid substitutions thereof, and light chain CDR1, CDR2 and CDR3 domains having the sequences set forth in SEQ ID NO:530, SEQ ID NO:531, and SEQ ID NO:532, respectively, and conservative amino acid substitutions thereof. In some embodiments, the antibody competes for binding with, and/or binds to the same epitope on CTLA-4 as any of the aforementioned antibodies.

In some embodiments, the CTLA-4 inhibitor is an anti-CTLA-4 biosimilar monoclonal antibody approved by drug regulatory authorities with reference to tremelimumab. In some embodiments, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tremelimumab. In some embodiments, the one or more post-translational modifications are selected from one or more of: glycosylation, oxidation, deamidation, and truncation. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is tremelimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab.

TABLE 25
Amino acid sequences for tremelimumab.

| Identifier | Sequence (One-Letter Amino Acid Symbols) | |
|---|---|---|
| SEQ ID NO: 523 | QVQLVESGGG VVQPGRSLRL SCAASGFTFS SYGMHWVRQA PGKGLEWVAV IWYDGSNKYY | 1 |
| tremelimumab | ADSVKGRFTI SRDNSKNTLY IQMNSLRAED TAVYYCARDP RGATLYYYYY GMDVWGQGTT | 61 |
| heavy chain | VTVSSASTKG PSVFPLAPCS RSTSESTAAL GCLVKDYFPE PVTVSWNSGA LTSGVHTFPA | 121 |
| | VLQSSGLYSI SSVVTVPSSN FGTQTYTCNV DHKPSNTKVD KTVERKCCVE CPPCPAPPVA | 181 |
| | GPSVFLFPPK PKDTLMISRT PEVTCVVVDV SHEDPEVQFN WYVDGVEVHN AKTKPREEQF | 241 |
| | NSTFRVVSVI TVVHQDWING KEYKCKVSNK GLPAPIEKTI SKTKGQPREP QVYTLPPSRE | 301 |
| | EMTKNQVSLT CLVKGFYPSD IAVEWESNG? PENNYKTTPP MLDSDGSFFL YSKLTVDKSR | 361 |
| | WQQGNVFSCS VMHEALHNHY TQKSLSLSPG K | 421 |
| SEQ ID NO: 524 | DIQMTQSPSS LSASVGDRVT ITCRASQSIN SYLDWYQQKP GKAPKLLIYA ASSLQSGVPS | 1 |
| tremelimumab | RFSGSGSGTD FTLTISSLQP EDFATYYCQ2 YYSTPFTFGP GTKVEIKRTV AAPSVFIFPP | 61 |
| light chain | SDEQLKSGTA SVVCLLNNFY PREAKVQWKV DNALQSGNSQ ESVTEQDSKD STYSLSSTLT | 121 |
| | LSKADYEKHK VYACEVTHQG LSSPVTKSFN RGEC | 181 |
| SEQ ID NO: 525 | GVVQPGRSLR LSCAASGFTF SSYGMHWVR? APGKGLEWVA VIWYDGSNKY YADSVKGRFT | 1 |
| tremelimumab | ISRDNSKNTI YLQMNSLRAE DTAVYYCARD PRGATLYYYY YGMDVWGQGT TVTVSSASTK | 61 |
| variable heavy | GPSVFPLAPC SRSTSESTAA LGCLVKDYFP EPVTVSWNSG ALTSGVH | 121 |
| chain | | |
| SEQ ID NO:526 | PSSLSASVGD RVTITCRASQ SINSYLDWYZ QKPGKAPKLL IYAASSLQSG VPSRFSGSGS | 1 |
| tremelimumab | GTDFTLTISS LQPEDFATYY CQQYYSTPFT FGPGTKVEIK RTVAAPSVFI FPPSDEQLKS | 61 |
| variable light | GTASVVCLLN NFYPREAKV | 121 |
| chain | | |
| SEQ ID NO: 527 | GFTFSSYGMH | 10 |
| tremel imumab | | |
| heavy chain | | |
| CDR1 | | |
| SEQ ID NO: 528 | VIWYDGSNKY YADSV | 15 |
| tremel imumab | | |
| heavy chain | | |
| CDR2 | | |
| SEQ ID NO: 529 | DPRGATLYYY YYGMDV | 16 |
| tremel imumab | | |
| heavy chain | | |
| CDR3 | | |
| SEQ ID NO: 530 | RASQSINSYL D | 11 |
| tremelimumab | | |
| light chain | | |
| CDR1 | | |
| SEQ ID NO: 531 | AASSLQS | 7 |
| tremel imumab | | |
| light chain | | |
| CDR2 | | |
| SEQ ID NO: 532 | QQYYSTPFT | 9 |
| tremel imumab | | |
| light chain | | |
| CDR3 | | |

In some embodiments, the CTLA-4 inhibitor is tremelimumab or a biosimilar thereof, and the tremelimumab is administered at a dose of about 0.5 mg/kg to about 10 mg/kg. In some embodiments, the CTLA-4 inhibitor is tremelimumab or a biosimilar thereof, and the tremelimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is tremelimumab or a biosimilar thereof, and the tremelimumab is administered at a dose of about 200 mg to about 500 mg. In some embodiments, the CTLA-4 inhibitor is tremelimumab or a biosimilar thereof, and the tremelimumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is tremelimumab or a biosimilar thereof, and the tremelimumab is administered every 2 weeks, every 3 weeks, every 4 weeks, every 5 weeks, or every 6 weeks. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is zalifrelimab from Adgenus, or biosimilars, antigen-binding fragments, conjugates, or variants thereof. Zalifrelimab is a fully human monoclonal antibody. Zalifrelimab is assigned Chemical Abstracts Service (CAS) registry number 2148321-69-9 and is also known as also known as AGEN1884. The preparation and properties of zalifrelimab are described in U.S. Pat. No. 10,144,779 and US Patent Application Publication No. US2020/0024350 A1, the disclosures of which are incorporated by reference herein.

In some embodiments, a CTLA-4 inhibitor comprises a heavy chain given by SEQ ID NO:533 and a light chain given by SEQ ID NO:534. In some embodiments, a CTLA-4 inhibitor comprises heavy and light chains having the sequences shown in SEQ ID NO: 533 and SEQ ID NO: 534, respectively, or antigen binding fragments, Fab fragments, single-chain variable fragments (scFv), variants, or conjugates thereof. In some embodiments, a CTLA-4 inhibitor comprises heavy and light chains that are each at least 99% identical to the sequences shown in SEQ ID NO: 533 and SEQ ID NO: 534, respectively. In some embodiments, a CTLA-4 inhibitor comprises heavy and light chains that are each at least 98% identical to the sequences shown in SEQ ID NO: 533 and SEQ ID NO: 534, respectively. In some embodiments, a CTLA-4 inhibitor comprises heavy and light chains that are each at least 97% identical to the sequences shown in SEQ ID NO: 533 and SEQ ID NO: 534, respectively. In some embodiments, a CTLA-4 inhibitor comprises heavy and light chains that are each at least 96% identical to the sequences shown in SEQ ID NO: 533 and SEQ ID NO: 534, respectively. In some embodiments, a CTLA-4 inhibitor comprises heavy and light chains that are each at least 95% identical to the sequences shown in SEQ ID NO: 533 and SEQ ID NO: 534, respectively.

In some embodiments, the CTLA-4 inhibitor comprises the heavy and light chain CDRs or variable regions (VRs) of zalifrelimab. In some embodiments, the CTLA-4 inhibitor heavy chain variable region (V_{H}) comprises the sequence shown in SEQ ID NO:535, and the CTLA-4 inhibitor light chain variable region (V_{L}) comprises the sequence shown in SEQ ID NO:536, and conservative amino acid substitutions thereof. In some embodiments, a CTLA-4 inhibitor comprises V_{H} and V_{L} regions that are each at least 99% identical to the sequences shown in SEQ ID NO:535 and SEQ ID NO:536, respectively. In some embodiments, a CTLA-4 inhibitor comprises V_{H} and V_{L} regions that are each at least 98% identical to the sequences shown in SEQ ID NO:535 and SEQ ID NO:536, respectively. In some embodiments, a CTLA-4 inhibitor comprises V_{H} and V_{L} regions that are each at least 97% identical to the sequences shown in SEQ ID NO:535 and SEQ ID NO:536, respectively. In some embodiments, a CTLA-4 inhibitor comprises V_{H} and V_{L} regions that are each at least 96% identical to the sequences shown in SEQ ID NO:535 and SEQ ID NO:536, respectively. In some embodiments, a CTLA-4 inhibitor comprises V_{H} and V_{L} regions that are each at least 95% identical to the sequences shown in SEQ ID NO:535 and SEQ ID NO:536, respectively.

In some embodiments, a CTLA-4 inhibitor comprises the heavy chain CDR1, CDR2 and CDR3 domains having the sequences set forth in SEQ ID NO:536, SEQ ID NO:538, and SEQ ID NO:539, respectively, and conservative amino acid substitutions thereof, and light chain CDR1, CDR2 and CDR3 domains having the sequences set forth in SEQ ID NO:540, SEQ ID NO:541, and SEQ ID NO:542, respectively, and conservative amino acid substitutions thereof. In some embodiments, the antibody competes for binding with, and/or binds to the same epitope on CTLA-4 as any of the aforementioned antibodies.

In some embodiments, the CTLA-4 inhibitor is a CTLA-4 biosimilar monoclonal antibody approved by drug regulatory authorities with reference to zalifrelimab. In some embodiments, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is zalifrelimab. In some embodiments, the one or more post-translational modifications are selected from one or more of: glycosylation, oxidation, deamidation, and truncation. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is zalifrelimab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is zalifrelimab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is zalifrelimab.

TABLE 26
Amino acid sequences for zalifrelimab.

| Identifier | Sequence (One-Letter Amino Acid Symbols) | |
|---|---|---|
| SEQ ID NO: 533 | EVQLVESGGG LVKPGGSLRL SCAASGFTFS SYSMNWVRQA PGKGLEWVSS ISSSSSYIYY | 1 |
| zalifrelimab | ADSVKGRFTI SRDNAKNSLY IQMNSLRAED TAVYYCARVG LMGPFDIWGQ GTMVTVSSAS | 61 |
| heavy chain | TKGPSVFPLA PSSKSTSGGT AALGCLVKDY FPEPVTVSWN SGALTSGVHT FPAVLQSSGL | 121 |
| | YSLSSVVTVP SSSLGTQTYI CNVNHKPSNT KVDKRVEPKS CDKTHTCPPC PAPELLGGPS | 181 |
| | VFLFPPKPKD TLMISRTPEV TCVVVDVSHE DPEVKFNWYV DGVEVHNAKT KPREEQYNST | 241 |
| | YRVVSVLTVI HQDWINGKEY KCKVSNKALP APIEKTISKA KGQPREPQVY TLPPSREEMT | 301 |
| | KNQVSLTCLV KGFYPSDIAV EWESNGQPEN NYKTTPPVLD SDGSFFLYSK LTVDKSRWQQ | 361 |
| | GNVFSCSVMH EALHNHYTQK SLSLSPGK | 421 |
| SEQ ID NO: 534 | EIVLTQSPGT LSLSPGERAT LSCRASQSVS RYLGWYQQKP GQAPRLLIYG ASTRATGIPD | 1 |
| zalifrelimab | RFSGSGSGTD FTLTITRLEP EDFAVYYCQQ YGSSPWTFGQ GTKVEIKRTV AAPSVFIFPP | 61 |
| light chain | SDEQLKSGTA SVVCLLNNFY PREAKVQWKV DNALQSGNSQ ESVTEQDSKD STYSLSSTLT | 121 |
| | LSKADYEKHK VYACEVTHQG LSSPVTKSFN RGEC | 181 |
| SEQ ID NO: 535 | EVQLVESGGG LVKPGGSLRL SCAASGFTFS SYSMNWVRQA PGKGLEWVSS ISSSSSYIYY | 1 |
| zalifrelimab | ADSVKGRFTI SRDNAKNSLY IQMNSLRAED TAVYYCARVG LMGPFDIWGQ GTMVTVSS | 61 |
| variable heavy | | |
| chain | | |
| SEQ ID NO: 536 | EIVLTQSPGT LSLSPGERAT LSCRASQSVS RYLGWYQQKP GQAPRLLIYG ASTRATGIPD | 1 |
| zalifrelimab | RFSGSGSGTD FTLTITRLEP EDFAVYYCQQ YGSSPWTFGQ GTKVEIK | 61 |
| variable light | | |
| chain | | |
| SEQ ID NO: 537 | GFTFSSYS | 8 |
| zalifrelimab | | |
| heavy chain | | |
| CDR1 | | |
| SEQ ID NO: 538 | ISSSSSYI | 8 |
| zalifrelimab | | |
| heavy chain | | |
| CDR2 | | |
| SEQ ID NO: 539 | ARVGLMGPFD I | 11 |
| zalifrelimab | | |
| heavy chain | | |
| CDR3 | | |
| SEQ ID NO:540 | QSVSRY | 6 |
| zalifrelimab | | |
| light chain | | |
| CDR1 | | |
| SEQ ID NO: 541 | GAS | 3 |
| zalifrelimab | | |
| light chain | | |
| CDR2 | | |
| SEQ ID NO: 542 | QQYGSSPWT | 9 |
| zalifrelimab | | |
| light chain | | |
| CDR3 | | |

Examples of additional anti-CTLA-4 antibodies includes, but are not limited to: AGEN1181, BMS-986218, BCD-145, ONC-392, CS1002, REGN4659, and ADG116, which are known to one of ordinary skill in the art.

In some embodiments, the anti-CTLA-4 antibody is an anti-CTLA-4 antibody disclosed in any of the following patent publications (incorporated herein by reference): US2019/0048096A1; US2020/0223907; US2019/0201334; US2019/0201334; US2005/0201994; EP 1212422 B1; WO2018204760; WO2018204760; WO2001014424; WO2004035607; WO2003086459; WO2012120125; WO2000037504; WO2009100140; WO200609649; WO2005092380; WO2007123737; WO2006029219; WO20100979597; WO200612168; and WO1997020574. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7,109,003, and 7,132,281; incorporated herein by reference). In some embodiments, the anti-CTLA-4 antibody is an, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin. Oncol., 22 (145): Abstract No. 2505 (2004) (antibody CP-675206); Mokyr et al., Cancer Res., 58:5301-5304 (1998) (incorporated herein by reference).

In some embodiments, the CTLA-4 inhibitor is a CTLA-4 ligand as disclosed in WO1996040915 (incorporated herein by reference).

In some embodiments, the CTLA-4 inhibitor is a nucleic acid inhibitor of CTLA-4 expression. For example, anti-CTLA-4 RNAi molecules may take the form of the molecules described by Mello and Fire in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA-4 RNAi molecules take the form of double stranded RNAi molecules described by Tuschl in European Patent No. EP 1309726 (incorporated herein by reference). In some instances, the anti-CTLA-4 RNAi molecules take the form of double stranded RNAi molecules described by Tuschl in U.S. Pat. Nos. 7,056,704 and 7,078,196 (incorporated herein by reference). In some embodiments, the CTLA-4 inhibitor is an aptamer described in PCT Publication No. WO2004081021 (incorporated herein by reference).

In other embodiments, the anti-CTLA-4 RNAi molecules of the present invention are RNA molecules described by Crooke in U.S. Pat. Nos. 5,898,031, 6,107,094, 7,432,249, and 7,432,250, and European Application No. EP 0928290 (incorporated herein by reference).

5. Lymphodepletion Preconditioning of Patients

In some embodiments, the invention includes a method of treating a cancer with a population of TILs, wherein a patient is pre-treated with non-myeloablative chemotherapy prior to an infusion of TILs according to the present disclosure. In some embodiments, the invention includes a population of TILs for use in the treatment of cancer in a patient which has been pre-treated with non-myeloablative chemotherapy. In some embodiments, the population of TILs is for administration by infusion. In some embodiments, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In some embodiments, after non-myeloablative chemotherapy and TIL infusion (at day 0) according to the present disclosure, the patient receives an intravenous infusion of IL-2 (aldesleukin, commercially available as PROLEUKIN) intravenously at 720,000 IU/kg every 8 hours to physiologic tolerance. In certain embodiments, the population of TILs is for use in treating cancer in combination with IL-2, wherein the IL-2 is administered after the population of TILs.

Experimental findings indicate that lymphodepletion prior to adoptive transfer of tumor-specific T lymphocytes plays a key role in enhancing treatment efficacy by eliminating regulatory T cells and competing elements of the immune system (‘cytokine sinks’). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et Clin. Oncol. 2005, 23, 2346-2357, all of which are incorporated by reference herein in their entireties.

In some embodiments, the fludarabine is administered at a concentration of 0.5 μg/mL to 10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL, to 10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide together to a patient. In some embodiments, fludarabine is administered at 25 mg/m²/day i.v. and cyclophosphamide is administered at 250 mg/m²/day i.v. over 4 days.

In some embodiments, the lymphodepletion is performed by administration of cyclophosphamide at a dose of 60 mg/m²/day for two days followed by administration of fludarabine at a dose of 25 mg/m²/day for five days.

In some embodiments, the lymphodepletion is performed by administration of cyclophosphamide at a dose of 60 mg/m²/day for two days and administration of fludarabine at a dose of 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days and administration of fludarabine at a dose of about 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days and administration of fludarabine at a dose of about 15 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of 60 mg/m²/day and fludarabine at a dose of 25 mg/m²/day for two days followed by administration of fludarabine at a dose of 25 mg/m²/day for three days.

In some embodiments, the cyclophosphamide is administered with mesna. In some embodiments, mesna is administered at 15 mg/kg. In some embodiments where mesna is infused, and if infused continuously, mesna can be infused over approximately 2 hours with cyclophosphamide (on Days −5 and/or −4), then at a rate of 3 mg/kg/hour for the remaining 22 hours over the 24 hours starting concomitantly with each cyclophosphamide dose.

In some embodiments, the lymphodepletion comprises the step of treating the patient with an IL-2 regimen starting on the day after administration of the third population of TILs to the patient.

In some embodiments, the lymphodepletion comprises the step of treating the patient with an IL-2 regimen starting on the same day as administration of the third population of TILs to the patient.

In some embodiments, the lymphodeplete comprises 5 days of preconditioning treatment. In some embodiments, the days are indicated as days −5 through −1, or Day 0 through Day 4. In some embodiments, the regimen comprises cyclophosphamide on days −5 and −4 (i.e., days 0 and 1). In some embodiments, the regimen comprises intravenous cyclophosphamide on days −5 and −4 (i.e., days 0 and 1). In some embodiments, the regimen comprises 60 mg/kg intravenous cyclophosphamide on days −5 and −4 (i.e., days 0 and 1). In some embodiments, the cyclophosphamide is administered with mesna. In some embodiments, the regimen further comprises fludarabine. In some embodiments, the regimen further comprises intravenous fludarabine. In some embodiments, the regimen further comprises 25 mg/m² intravenous fludarabine. In some embodiments, the regimen further comprises 25 mg/m² intravenous fludarabine on days −5 and −1 (i.e., days 0 through 4). In some embodiments, the regimen further comprises 25 mg/m² intravenous fludarabine on days −5 and −1 (i.e., days 0 through 4).

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day.

In some embodiments, the non-myeloablative lymphodepletion regimen is administered according to Table 27.

TABLE 27
Exemplary lymphodepletion and treatment regimen.

| | Day | | | | | | | | | |
|---|---|---|---|---|---|---|---|---|---|---|
| | −5 | −4 | −3 | −2 | −1 | 0 | 1 | 2 | 3 | 4 |
| Cyclophosphamide 60 mg/kg | X | X | | | | | | | | |
| Mesna (as needed) | X | X | | | | | | | | |
| Fludarabine 25 mg/m²/day | X | X | X | X | X | | | | | |
| TIL infusion | | | | | | X | | | | |

In some embodiments, the non-myeloablative lymphodepletion regimen is administered according to Table 28.

TABLE 28
Exemplary lymphodepletion and treatment regimen.

| | Day | | | | | | | | |
|---|---|---|---|---|---|---|---|---|---|
| | −4 | −3 | −2 | −1 | 0 | 1 | 2 | 3 | 4 |
| Cyclophosphamide 60 mg/kg | X | X | | | | | | | |
| Mesna (as needed) | X | X | | | | | | | |
| Fludarabine 25 mg/m²/day | X | X | X | X | | | | | |
| TIL infusion | | | | | X | | | | |

In some embodiments, the non-myeloablative lymphodepletion regimen is administered according to Table 29.

TABLE 29
Exemplary lymphodepletion and treatment regimen.

| | Day | | | | | | | |
|---|---|---|---|---|---|---|---|---|
| | −3 | −2 | −1 | 0 | 1 | 2 | 3 | 4 |
| Cyclophosphamide 60 mg/kg | X | X | | | | | | |
| Mesna (as needed) | X | X | | | | | | |
| Fludarabine 25 mg/m²/day | X | X | X | | | | | |
| TIL infusion | | | | X | | | | |

In some embodiments, the non-myeloablative lymphodepletion regimen is administered according to Table 30.

TABLE 30
Exemplary lymphodepletion and treatment regimen.

| | Day | | | | | | | | | |
|---|---|---|---|---|---|---|---|---|---|---|
| | −5 | −4 | −3 | −2 | −1 | 0 | 1 | 2 | 3 | 4 |
| Cyclophosphamide 60 mg/kg | X | X | | | | | | | | |
| Mesna (as needed) | X | X | | | | | | | | |
| Fludarabine 25 mg/m²/day | | | X | | X | | | | | |
| TIL infusion | | | | | | X | | | | |

In some embodiments, the non-myeloablative lymphodepletion regimen is administered according to Table 31.

TABLE 31
Exemplary lymphodepletion and treatment regimen.

| | Day | | | | | | | | | |
|---|---|---|---|---|---|---|---|---|---|---|
| | −5 | −4 | −3 | −2 | −1 | 0 | 1 | 2 | 3 | 4 |
| Cyclophosphamide 300 mg/kg | X | X | | | | | | | | |
| Mesna (as needed) | X | X | | | | | | | | |
| Fludarabine 30 mg/m²/day | X | | X | X | X | | | | | |
| TIL infusion | | | | | | X | | | | |

In some embodiments, the non-myeloablative lymphodepletion regimen is administered according to Table 32.

TABLE 32
Exemplary lymphodepletion and treatment regimen.

| | Day | | | | | | | | |
|---|---|---|---|---|---|---|---|---|---|
| | −4 | −3 | −2 | −1 | 0 | 1 | 2 | 3 | 4 |
| Cyclophosphamide 300 mg/kg | X | X | | | | | | | |
| Mesna (as needed) | X | X | | | | | | | |
| Fludarabine 30 mg/m²/day | X | X | X | X | | | | | |
| TIL infusion | | | | | X | | | | |

In some embodiments, the non-myeloablative lymphodepletion regimen is administered according to Table 33.

TABLE 33
Exemplary lymphodepletion and treatment regimen.

| | Day | | | | | | | |
|---|---|---|---|---|---|---|---|---|
| | −3 | −2 | −1 | 0 | 1 | 2 | 3 | 4 |
| Cyclophosphamide 300 mg/kg | X | X | | | | | | |
| Mesna (as needed) | X | X | | | | | | |
| Fludarabine 30 mg/m²/day | X | X | X | | | | | |
| TIL infusion | | | | X | | | | |

In some embodiments, the non-myeloablative lymphodepletion regimen is administered according to Table 34.

TABLE 34
Exemplary lymphodepletion and treatment regimen.

| | Day | | | | | | | | | |
|---|---|---|---|---|---|---|---|---|---|---|
| | −5 | −4 | −3 | −2 | −1 | 0 | 1 | 2 | 3 | 4 |
| Cyclophosphamide 300 mg/kg | X | X | | | | | | | | |
| Mesna (as needed) | X | X | | | | | | | | |
| Fludarabine 30 mg/m²/day | | | X | X | X | | | | | |
| TIL infusion | | | | | | X | | | | |

6. IL-2 Regimens

In some embodiments, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of the therapeutic population of TILs, wherein the aldesleukin or a biosimilar or variant thereof is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total. In some embodiments, IL-2 is administered in 1, 2, 3, 4, 5, or 6 doses. In some embodiments, IL-2 is administered at a maximum dosage of up to 6 doses. In some embodiments, the high-dose IL-2 regimen is adapted for pediatric use. In some embodiments, a dose of 600,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 400,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 300,000 international units (IU)/kg of aldesleukin every 8-12 hours for up to a maximum of 6 doses is used. In some embodiments, a dose of 200,000 international units (IU)/kg of aldesleukin every 8-12 hours for up to a maximum of 6 doses is used. In some embodiments, a dose of 100,000 international units (IU)/kg of aldesleukin every 8-12 hours for up to a maximum of 6 doses is used.

In some embodiments, the IL-2 regimen comprises a decrescendo IL-2 regimen. Decrescendo IL-2 regimens have been described in O'Day, et al., J. Clin. Oncol. 1999, 17, 2752-61 and Eton, et al., Cancer 2000, 88, 1703-9, the disclosures of which are incorporated herein by reference. In some embodiments, a decrescendo IL-2 regimen comprises 18×10⁶ IU/m² administered intravenously over 6 hours, followed by 18×10⁶ IU/m² administered intravenously over 12 hours, followed by 18×10⁶ IU/m² administered intravenously over 24 hrs, followed by 4.5×10⁶ IU/m² administered intravenously over 72 hours. This treatment cycle may be repeated every 28 days for a maximum of four cycles. In some embodiments, a decrescendo IL-2 regimen comprises 18,000,000 IU/m² on day 1, 9,000,000 IU/m² on day 2, and 4,500,000 IU/m² on days 3 and 4.

In an embodiment, the IL-2 regimen comprises a low-dose IL-2 regimen. Any low-dose IL-2 regimen known in the art may be used, including the low-dose IL-2 regimens described in Dominguez-Villar and Hafler, Nat. Immunology 2000, 19, 665-673; Hartemann, et al., Lancet Diabetes Endocrinol. 2013, 1, 295-305; and Rosenzwaig, et al., Ann. Rheum. Dis. 2019, 78, 209-217, the disclosures of which are incorporated herein by reference. In an embodiment, a low-dose IL-2 regimen comprises 18×10⁶ IU per m² of aldesleukin, or a biosimilar or variant thereof, per 24 hours, administered as a continuous infusion for 5 days, followed by 2-6 days without IL-2 therapy, optionally followed by an additional 5 days of intravenous aldesleukin or a biosimilar or variant thereof, as a continuous infusion of 18×10⁶ IU per m² per 24 hours, optionally followed by 3 weeks without IL-2 therapy, after which additional cycles may be administered.

In some embodiments, IL-2 is administered at a maximum dosage of up to 6 doses. In some embodiments, the high-dose IL-2 regimen is adapted for pediatric use. In some embodiments, a dose of 600,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 400,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 300,000 international units (IU)/kg of aldesleukin every 8-12 hours for up to a maximum of 6 doses is used. In some embodiments, a dose of 200,000 international units (IU)/kg of aldesleukin every 8-12 hours for up to a maximum of 6 doses is used. In some embodiments, a dose of 100,000 international units (IU)/kg of aldesleukin every 8-12 hours for up to a maximum of 6 doses is used.

In some embodiments, the IL-2 regimen comprises administration of pegylated IL-2 every 1, 2, 4, 6, 7, 14 or 21 days at a dose of 0.10 mg/day to 50 mg/day. In some embodiments, the IL-2 regimen comprises administration of bempegaldesleukin, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of THOR-707, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of nemvaleukin alfa, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of an IL-2 fragment engrafted onto an antibody backbone. In some embodiments, the IL-2 regimen comprises administration of an antibody-cytokine engrafted protein that binds the IL-2 low affinity receptor. In some embodiments, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the antibody cytokine engrafted protein preferentially expands T effector cells over regulatory T cells. In some embodiments, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the IL-2 molecule is a mutein, and wherein the antibody cytokine engrafted protein preferentially expands T effector cells over regulatory T cells. In some embodiments, the IL-2 regimen comprises administration of an antibody comprising a heavy chain selected from the group consisting of SEQ ID NO:559 and SEQ ID NO:568 and a light chain selected from the group consisting of SEQ ID NO:567 and SEQ ID NO:569, or a fragment, variant, or biosimilar thereof, every 1, 2, 4, 6, 7, 14 or 21 days at a dose of 0.10 mg/day to 50 mg/day.

In some embodiments, the antibody cytokine engrafted protein described herein has a longer serum half-life that a wild-type IL-2 molecule such as, but not limited to, aldesleukin (Proleukin®) or a comparable molecule.

In some embodiments, the TIL infusion used with the foregoing embodiments of myeloablative lymphodepletion regimens may be any TIL composition described herein and may also include infusions of MILs and PBLs in place of the TIL infusion, as well as the addition of IL-2 regimens and administration of co-therapies (such as PD-1 and/or PD-L1 inhibitors and/or CTLA-4 inhibitors) as described herein.

6. Additional Methods of Treatment

In other embodiments, the invention provides a method for treating a subject with cancer comprising administering to the subject a therapeutically effective dosage of the therapeutic TIL population described in any of the preceding paragraphs as applicable above.

In other embodiments, the invention provides a method for treating a subject with cancer comprising administering to the subject a therapeutically effective dosage of the TIL composition described in any of the preceding paragraphs as applicable above.

In other embodiments, the invention provides the method for treating a subject with cancer described in any of the preceding paragraphs as applicable above modified such that prior to administering the therapeutically effective dosage of the therapeutic TIL population and the TIL composition described in any of the preceding paragraphs as applicable above, respectively, a non-myeloablative lymphodepletion regimen has been administered to the subject.

In other embodiments, the invention provides the method for treating a subject with cancer described in any of the preceding paragraphs as applicable above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the method for treating a subject with cancer described in any of the preceding paragraphs as applicable above modified to further comprise the step of treating the subject with a high-dose IL-2 regimen starting on the day after administration of the TIL cells to the subject.

In other embodiments, the invention provides the method for treating a subject with cancer described in any of the preceding paragraphs as applicable above modified such that the high-dose IL-2 regimen comprises 600,000 or 720,000 IU/kg administered as a 15-minute bolus intravenous infusion every eight hours until tolerance.

In other embodiments, the invention provides the method for treating a subject with cancer described in any of the preceding paragraphs as applicable above modified such that the cancer is a solid tumor.

In other embodiments, the invention provides the method for treating a subject with cancer described in any of the preceding paragraphs as applicable above modified such that the cancer is melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the method for treating a subject with cancer described in any of the preceding paragraphs as applicable above modified such that the cancer is melanoma, HNSCC, cervical cancers, NSCLC, glioblastoma (including GBM), and gastrointestinal cancer.

In other embodiments, the invention provides the method for treating a subject with cancer described in any of the preceding paragraphs as applicable above modified such that the cancer is melanoma. In other embodiments, the invention provides the method for treating a subject with cancer described in any of the preceding paragraphs as applicable above modified such that the cancer is refractory or unresectable melanoma.

In other embodiments, the invention provides the method for treating a subject with cancer described in any of the preceding paragraphs as applicable above modified such that the cancer is HNSCC.

In other embodiments, the invention provides the method for treating a subject with cancer described in any of the preceding paragraphs as applicable above modified such that the cancer is a cervical cancer.

In other embodiments, the invention provides the method for treating a subject with cancer described in any of the preceding paragraphs as applicable above modified such that the cancer is NSCLC.

In other embodiments, the invention provides the method for treating a subject with cancer described in any of the preceding paragraphs as applicable above modified such that the cancer is glioblastoma (including GBM).

In other embodiments, the invention provides the method for treating a subject with cancer described in any of the preceding paragraphs as applicable above modified such that the cancer is gastrointestinal cancer.

In other embodiments, the invention provides the method for treating a subject with cancer described in any of the preceding paragraphs as applicable above modified such that the cancer is a hypermutated cancer.

In other embodiments, the invention provides the method for treating a subject with cancer described in any of the preceding paragraphs as applicable above modified such that the cancer is a pediatric hypermutated cancer.

In other embodiments, the invention provides the therapeutic TIL population described in any of the preceding paragraphs as applicable above for use in a method for treating a subject with cancer comprising administering to the subject a therapeutically effective dosage of the therapeutic TIL population.

In other embodiments, the invention provides the TIL composition described in any of the preceding paragraphs as applicable above for use in a method for treating a subject with cancer comprising administering to the subject a therapeutically effective dosage of the TIL composition.

In other embodiments, the invention provides the therapeutic TIL population described in any of the preceding paragraphs as applicable above or the TIL composition described in any of the preceding paragraphs as applicable above modified such that prior to administering to the subject the therapeutically effective dosage of the therapeutic TIL population described in any of the preceding paragraphs as applicable above or the TIL composition described in any of the preceding paragraphs as applicable above, a non-myeloablative lymphodepletion regimen has been administered to the subject.

In other embodiments, the invention provides the therapeutic TIL population or the TIL composition described in any of the preceding paragraphs as applicable above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the therapeutic TIL population or the TIL composition described in any of the preceding paragraphs as applicable above modified to further comprise the step of treating patient with a high-dose IL-2 regimen starting on the day after administration of the TIL cells to the patient.

In other embodiments, the invention provides the therapeutic TIL population or the TIL composition described in any of the preceding paragraphs as applicable above modified such that the high-dose IL-2 regimen comprises 600,000 or 720,000 IU/kg administered as a 15-minute bolus intravenous infusion every eight hours until tolerance.

In other embodiments, the invention provides the therapeutic TIL population or the TIL composition described in any of the preceding paragraphs as applicable above modified such that the cancer is a solid tumor.

In other embodiments, the invention provides the therapeutic TIL population or the TIL composition described in any of the preceding paragraphs as applicable above modified such that the cancer is melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the therapeutic TIL population or the TIL composition described in any of the preceding paragraphs as applicable above modified such that the cancer is melanoma, HNSCC, cervical cancers, NSCLC, glioblastoma (including GBM), and gastrointestinal cancer.

In other embodiments, the invention provides the therapeutic TIL population or the TIL composition described in any of the preceding paragraphs as applicable above modified such that the cancer is melanoma.

In other embodiments, the invention provides the therapeutic TIL population or the TIL composition described in any of the preceding paragraphs as applicable above modified such that the cancer is HNSCC.

In other embodiments, the invention provides the therapeutic TIL population or the TIL composition described in any of the preceding paragraphs as applicable above modified such that the cancer is a cervical cancer.

In other embodiments, the invention provides the therapeutic TIL population or the TIL composition described in any of the preceding paragraphs as applicable above modified such that the cancer is NSCLC.

In other embodiments, the invention provides the therapeutic TIL population or the TIL composition described in any of the preceding paragraphs as applicable above modified such that the cancer is glioblastoma (including GBM).

In other embodiments, the invention provides the therapeutic TIL population or the TIL composition described in any of the preceding paragraphs as applicable above modified such that the cancer is gastrointestinal cancer.

In other embodiments, the invention provides the therapeutic TIL population or the TIL composition described in any of the preceding paragraphs as applicable above modified such that the cancer is a hypermutated cancer.

In other embodiments, the invention provides the therapeutic TIL population or the TIL composition described in any of the preceding paragraphs as applicable above modified such that the cancer is a pediatric hypermutated cancer.

In other embodiments, the invention provides the use of the therapeutic TIL population described in any of the preceding paragraphs as applicable above in a method of treating cancer in a subject comprising administering to the subject a therapeutically effective dosage of the therapeutic TIL population.

In other embodiments, the invention provides the use of the TIL composition described in any of the preceding paragraphs as applicable above in a method of treating cancer in a subject comprising administering to the subject a therapeutically effective dosage of the TIL composition.

In other embodiments, the invention provides the use of the therapeutic TIL population described any of the preceding paragraphs as applicable above or the TIL composition described in any of the preceding paragraphs as applicable above in a method of treating cancer in a subject comprising administering to the subject a non-myeloablative lymphodepletion regimen and then administering to the subject the therapeutically effective dosage of the therapeutic TIL population described in any of the preceding paragraphs as applicable above or the therapeutically effective dosage of the TIL composition described in any of the preceding paragraphs as applicable above.

In other embodiments, the invention provides the use of the therapeutic TIL population or the TIL composition described in any of the preceding paragraphs as applicable above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the use of the therapeutic TIL population or the TIL composition described in any of the preceding paragraphs as applicable above modified to further comprise the step of treating patient with a high-dose IL-2 regimen starting on the day after administration of the TIL cells to the patient.

In other embodiments, the invention provides the use of the therapeutic TIL population or the TIL composition described in any of the preceding paragraphs as applicable above modified such that the high-dose IL-2 regimen comprises 600,000 or 720,000 IU/kg administered as a 15-minute bolus intravenous infusion every eight hours until tolerance.

In other embodiments, the invention provides the use of the therapeutic TIL population or the TIL composition described in any of the preceding paragraphs as applicable above modified such that the cancer is a solid tumor.

In other embodiments, the invention provides the use of the therapeutic TIL population or the TIL composition described in any of the preceding paragraphs as applicable above modified such that the cancer is melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the use of the therapeutic TIL population or the TIL composition described in any of the preceding paragraphs as applicable above modified such that the cancer is melanoma, HNSCC, cervical cancers, NSCLC, glioblastoma (including GBM), and gastrointestinal cancer.

In other embodiments, the invention provides the use of the therapeutic TIL population or the TIL composition described in any of the preceding paragraphs as applicable above modified such that the cancer is melanoma.

In other embodiments, the invention provides the use of the therapeutic TIL population or the TIL composition described in any of the preceding paragraphs as applicable above modified such that the cancer is HNSCC.

In other embodiments, the invention provides the use of the therapeutic TIL population or the TIL composition described in any of the preceding paragraphs as applicable above modified such that the cancer is a cervical cancer.

In other embodiments, the invention provides the use of the therapeutic TIL population or the TIL composition described in any of the preceding paragraphs as applicable above modified such that the cancer is NSCLC.

In other embodiments, the invention provides the use of the therapeutic TIL population or the TIL composition described in any of the preceding paragraphs as applicable above modified such that the cancer is glioblastoma (including GBM).

In other embodiments, the invention provides the use of the therapeutic TIL population or the TIL composition described in any of the preceding paragraphs as applicable above modified such that the cancer is gastrointestinal cancer.

In other embodiments, the invention provides the use of the therapeutic TIL population or the TIL composition described in any of the preceding paragraphs as applicable above modified such that the cancer is a hypermutated cancer.

In other embodiments, the invention provides the use of the therapeutic TIL population or the TIL composition described in any of the preceding paragraphs as applicable above modified such that the cancer is a pediatric hypermutated cancer.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Preparation of Media for Pre-Rep and Rep Processes

This Example describes the procedure for the preparation of tissue culture media for use in protocols involving the culture of tumor infiltrating lymphocytes (TIL) derived from various tumor types including, but not limited to, metastatic melanoma, head and neck squamous cell carcinoma (HNSCC), ovarian carcinoma, triple-negative breast carcinoma, and lung adenocarcinoma. This media can be used for preparation of any of the TILs described in the present application and Examples.

### Preparation of CM1

Removed the following reagents from cold storage and warmed them in a 37° C. water bath: (RPMI1640, Human AB serum, 200 mM L-glutamine). Prepared CM1 medium according to Table 35 below by adding each of the ingredients into the top section of a 0.2 μm filter unit appropriate to the volume to be filtered. Stored at 4° C.

TABLE 35
Preparation of CM1

| Ingredient | Final concentration | | Final Volume 500 ml | | Final Volume IL | |
|---|---|---|---|---|---|---|
| RPMI1640 | NA | | 450 | ml | 900 | ml |
| Human AB serum, | 50 | ml | 100 | ml | | |
| heat-inactivated 10% | | | | | | |
| 200 mM L-glutamine | 2 | mM | 5 | ml | 10 | ml |
| 55 mM BME | 55 | μM | 0.5 | ml | 1 | ml |
| 50 mg/ml gentamicin | 50 | μg/ml | 0.5 | ml | 1 | ml |
| sulfate | | | | | | |

On the day of use, prewarmed required amount of CM1 in 37° C. water bath and add 6000 IU/ml IL-2.

Additional supplementation—as needed according to Table 36.

TABLE 36
Additional supplementation of CM1, as needed.

| Supplement | Stock concentration | Dilution | Final concentration |
|---|---|---|---|
| GlutaMAX ™ | 200 mM | 1:100 | 2 mM |
| Penicillin/ | 10,000 U/ml penicillin | 1:100 | 100 U/ml penicillin |
| streptomycin | 10,000 μg/ml | | 100 μg/ml |
| | streptomycin | | streptomycin |
| Amphotericin B | 250 μg/ml | 1:100 | 2.5 μg/ml |

### Preparation of CM2

Removed prepared CM1 from refrigerator or prepare fresh CM1 as per Table 35 above. Removed AIM-V® from refrigerator and prepared the amount of CM2 needed by mixing prepared CM1 with an equal volume of AIM-V® in a sterile media bottle. Added 3000 IU/ml IL-2 to CM2 medium on the day of usage. Made sufficient amount of CM2 with 3000 IU/ml IL-2 on the day of usage. Labeled the CM2 media bottle with its name, the initials of the preparer, the date it was filtered/prepared, the two-week expiration date and stored at 4° C. until needed for tissue culture.

### Preparation of CM3

Prepared CM3 on the day it was required for use. CM3 was the same as AIM-V® medium, supplemented with 3000 IU/ml IL-2 on the day of use. Prepared an amount of CM3 sufficient to experimental needs by adding IL-2 stock solution directly to the bottle or bag of AIM-V. Mixed well by gentle shaking. Labeled bottle with “3000 IU/ml IL-2” immediately after adding to the AIM-V. If there was excess CM3, stored it in bottles at 4° C. labeled with the media name, the initials of the preparer, the date the media was prepared, and its expiration date (7 days after preparation). Discarded media supplemented with IL-2 after 7 days storage at 4° C.

### Preparation of CM4

CM4 was the same as CM3, with the additional supplement of 2 mM GlutaMAX™ (final concentration). For every 1L of CM3, added 10 ml of 200 mM GlutaMAX™. Prepared an amount of CM4 sufficient to experimental needs by adding IL-2 stock solution and GlutaMAX™ stock solution directly to the bottle or bag of AIM-V. Mixed well by gentle shaking. Labeled bottle with “3000 IL/ml IL-2 and GlutaMAX” immediately after adding to the AIM-V. If there was excess CM4, stored it in bottles at 4° C. labeled with the media name, “GlutaMAX”, and its expiration date (7 days after preparation). Discarded media supplemented with IL-2 after 7-days storage at 4° C.

### Example 2: Preparation of IL-2 Stock Solution (Cellgenix)

This Example describes the process of dissolving purified, lyophilized recombinant human interleukin-2 into stock samples suitable for use in further tissue culture protocols, including all of those described in the present application and Examples, including those that involve using rhIL-2.

### Procedure

Prepared 0.2% Acetic Acid solution (HAc). Transferred 29 mL sterile water to a 50 mL conical tube. Added 1 mL 1N acetic acid to the 50 mL conical tube. Mixed well by inverting tube 2-3 times. Sterilized the HAc solution by filtration using a Steriflip filter

Prepared 1% HSA in PBS. Added 4 mL of 25% HSA stock solution to 96 mL PBS in a 150 mL sterile filter unit. Filtered solution. Stored at 4° C. For each vial of rhIL-2 prepared, fill out forms.

Prepared rhIL-2 stock solution (6×10⁶ IU/mL final concentration). Each lot of rhIL-2 was different and required information found in the manufacturer's Certificate of Analysis (COA), such as: 1) Mass of rhIL-2 per vial (mg). 2) Specific activity of rhIL-2 (IU/mg) and 3) Recommended 0.2% HAc reconstitution volume (mL).

Calculated the volume of 1% HSA required for rhIL-2 lot by using the equation below:

$$( \frac{Vial Mass ( mg ) \times Biological Activity ( \frac{IU}{mg} )}{6 \times {10}^{6} \frac{IU}{mL}} ) - HAc vol ( mL ) = 1 \% HSA vol ( mL )$$

For example, according to CellGenix's rhIL-2 lot 10200121 COA, the specific activity for the 1 mg vial is 25×10⁶ IU/mg. It recommends reconstituting the rhIL-2 in 2 mL 0.2% HAc.

$$( \frac{1 mg \times 25 \times {10}^{6} \frac{IU}{mg}}{6 \times {10}^{6} \frac{IU}{mL}} ) - 2 mL = 2.167 mL HSA$$

Wiped rubber stopper of IL-2 vial with alcohol wipe. Using a 16 G needle attached to a 3 mL syringe, injected recommended volume of 0.2% HAc into vial. Took care to not dislodge the stopper as the needle is withdrawn. Inverted vial 3 times and swirled until all powder was dissolved. Carefully removed the stopper and set aside on an alcohol wipe. Added the calculated volume of 1% HSA to the vial.

Storage of rhIL-2 solution. For short-term storage (<72 hrs), stored vial at 4° C. For long-term storage (>72 hrs), aliquoted vial into smaller volumes and stored in cryovials at −20° C. until ready to use. Avoided freeze/thaw cycles. Recorded expiration date of 6 months after date of preparation. Rh-IL-2 labels included vendor and catalog number, lot number, expiration date, operator initials, concentration and volume of aliquot.

### Example 3: Cryopreservation Process

This example describes the cryopreservation process method for TILs prepared with the abbreviated, closed procedure described in Example 9 using the CryoMed Controlled Rate Freezer, Model 7454 (Thermo Scientific).

The equipment used was as follows: aluminum cassette holder rack (compatible with CS750 freezer bags), cryostorage cassettes for 750 mL bags, low pressure (22 psi) liquid nitrogen tank, refrigerator, thermocouple sensor (ribbon type for bags), and CryoStore CS750 Freezing bags (OriGen Scientific).

The freezing process provided for a 0.5° C. rate from nucleation to −20° C. and 1° C. per minute cooling rate to −80° C. end temperature. The program parameters are as follows: Step 1-wait at 4° C.; Step 2: 1.0° C./min (sample temperature) to −4° C.; Step 3: 20.0° C./min (chamber temperature) to −45° C.; Step 4: 10.0° C./min (chamber temperature) to −10.0° C.; Step 5: 0.5° C./min (chamber temperature) to −20° C.; and Step 6: 1.0° C./min (sample temperature) to −80° C.

### Example 4: Tumor Expansion Processes with Defined Medium

The processes disclosed in Examples 1 through 7 are performed with substituting the CM1 and CM2 media with a defined medium according to the present invention (e.g., CTS™ OpTmizer™ T-Cell Expansion SFM, ThermoFisher, including for example DM1 and DM2).

### Example 5: Selection of PD-1+ TIL Using Nivolumab by Flow Cytometry Sorting and Expansion in Full-Scale for Clinical Manufacturing

### Purpose

This report describes the results from the expansion of PD-1-selected TIL using Nivolumab for the selection in full-scale manufacturing experiments described in the present Examples.

### SCOPE

The scope of work was to expand PD-1-selected TIL from melanoma or lung or head and neck or ovarian tumors.

On Day 0, tumor digest was equally distributed to two arms, and the tumor digest in each arm of the experiment was stained using either Nivolumab or anti-PD1 Clone #EH12.2H7 (Research grade) as the primary antibody, and FITC-conjugated anti-IgG4 secondary antibody. PD-1 expressing TIL from the stained populations were then selected by flow sorting. Two step expansion process was used to expand PD-1-selected TIL for full scale clinical manufacturing. The first step of expansion (“Activation”) was conducted from Day 0 to Day 11. The second step of expansion process (“Rapid Expansion Phase”, or “REP”, including Split on Day 16) were conducted from Day 11 to Day 22. The final product was harvested on Day 22.

For Small-Scale process ( 1/100th scale), Activation was initiated on Day 0 using 10% of the PD-1-selected TIL with the lowest sort result, and transferring that number of TIL from each sort into the respective G-Rex-10M flasks with Feeders and OKT-3 with IL-2 media. REP, Split, and Harvest were initiated per TP-19-004. A brief explanation of the associated timepoints is outlined below in the methods section.

For Full-Scale process, Activation was initiated on Day 0 using PD-1-selected TIL with the similar cell number, with 100e6 allogeneic feeder cells and 30 ng/mL OKT3 for 11 days. REP was initiated on Day 11 from the harvested product. REP (Day 11) and the subsequent Day 16 (Split) and Day 22 (Harvest) processes were performed per IOVA Manufacturing Batch Records. A brief explanation of the associated timepoints is outlined below in the Experimental design (Table 37).

The expanded final product TIL were assessed for cell growth, viability, phenotype, and function (IFN-γ and Granzyme-B secretion, CD107a mobilization upon stimulation).

Additional analysis was performed on the extended characterization data to establish the equivalence of EH12.2H7 and Nivolumab.

### Background Information

A previously developed protocol designed to select PD-1 expressing TIL from tumor digests using PE-conjugated anti-PD-1 antibody (Clone #EH12.2H7) to enrich the TIL product for autologous tumor-reactive T cells is provided in Example 21.

In the current study, Example 9 and Example 21 were adapted to obtain PD1-selected TIL using nivolumab as the anti-PD1 antibody in lieu of the PE-conjugated clone #EH12.2H7, and using FITC-conjugated anti-IgG4 antibody as secondary staining antibody.

### Experiment Design

Two small scale experiments and bulk control condition were conducted per TP-19-004.

One full scale experiment was conducted per Example 6.

Overview of Small scale and full scale were provided in Tables 37 and 38.

TABLE 37
Overview of Small-Scale PD-1-selected
TIL process in 1/100th scale

| Condition | 1/100th scale | |
|---|---|---|
| Activation (1/10^{th} scale) | Day 0: Activation | |
| TIL | 10% of PD-1-selected TIL | |
| Feeders | 10e6 | |
| CM1 | 100 | mL |
| IL-2 | 6000 | IU/mL |
| OKT3 (30 ng/ml) | 30 | ng/ml |
| G-Rex | 10M | |
| REP (1/100^{th} scale) | Day 11: REP | |
| TIL | 10% TVC | |
| Feeders | 50e6 | |
| CM2 | 50 | mL |
| IL-2 | 3000 | IU/mL |
| OKT3 (30 ng/ml) | 30 | ng/ml |

| | |
|---|---|
| G-Rex | 5M |
| Split (1/100^{th} scale) | Day 16: Volume reduce |
| | and split (TVC/10e6, |
| | round up) up to |
| | 5 × 5M flasks |
| REP Harvest (1/100^{th} scale) | Day 22: REP Harvest |
| Extrapolation Calculation: | |
| Activation | Multiply Activation |
| | Harvest TVC by 10 |
| REP, Split, Harvest | Multiply by REP Harvest |
| | by 100 × # |
| | of split flasks |

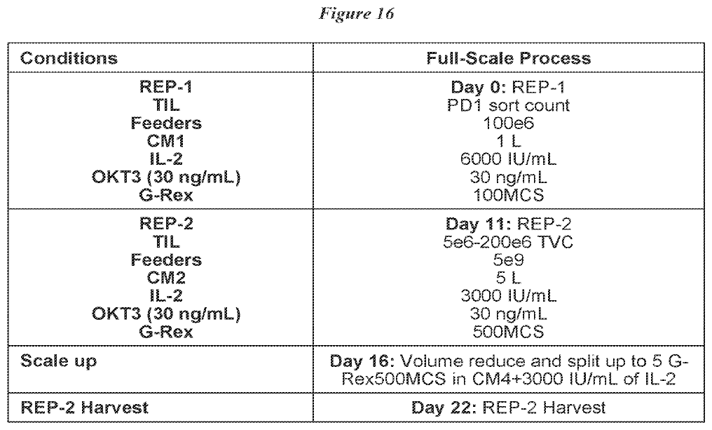

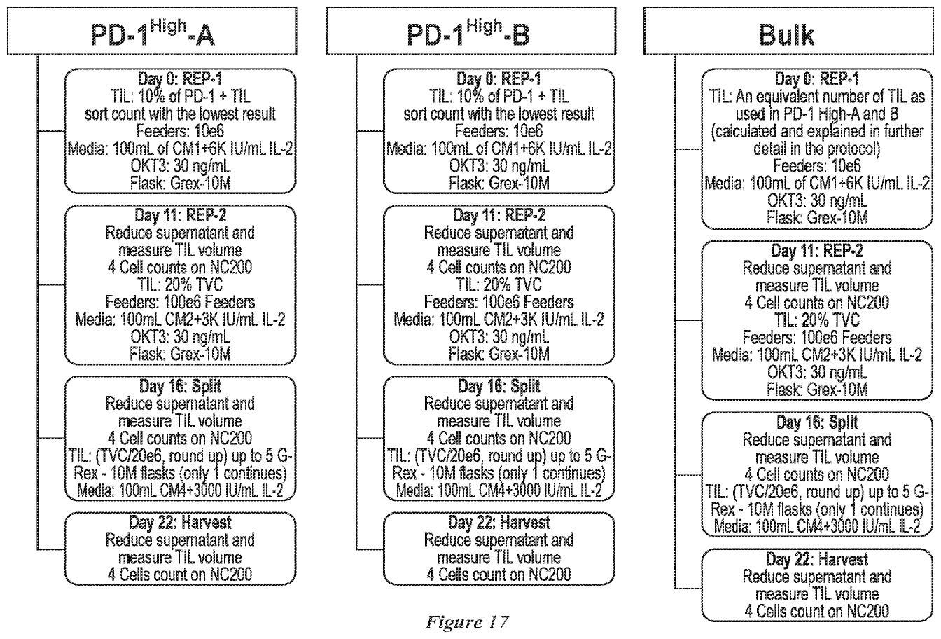

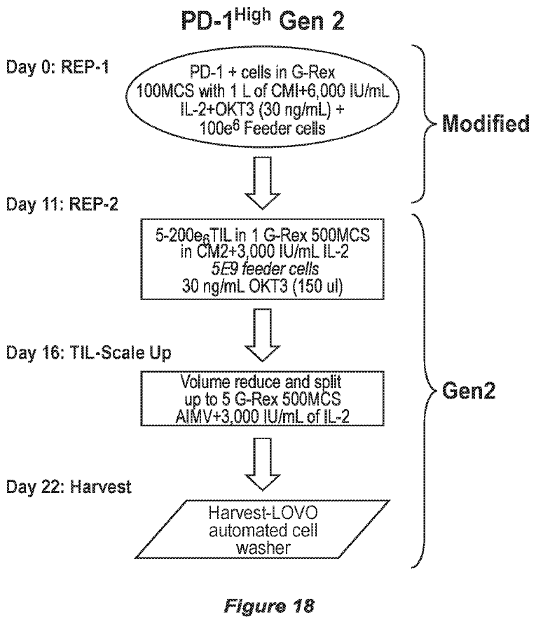

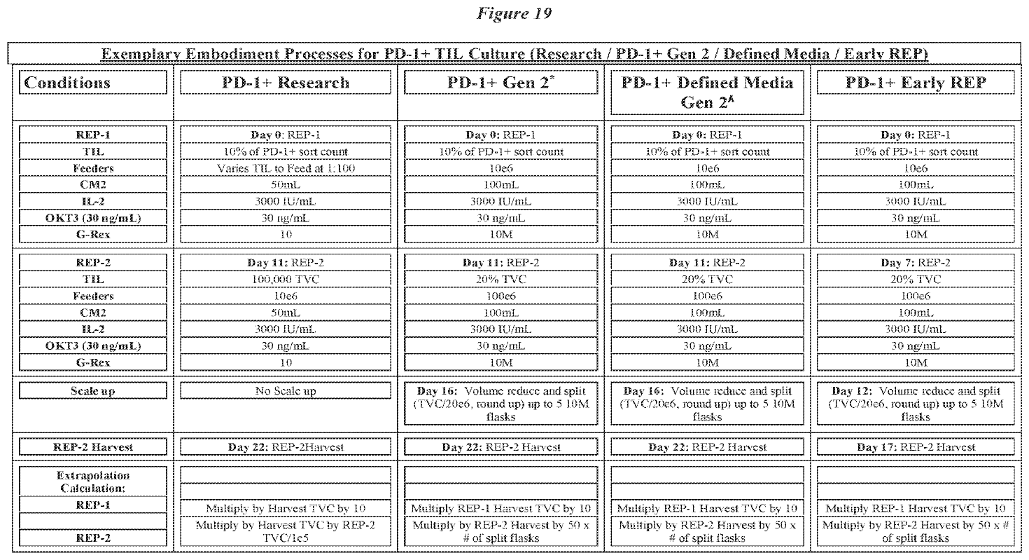

TABLE 38
Overview of Full-Scale PD-1-selected
TIL Process (See, also, FIG. 16)

| Conditions | Full Scale | |
|---|---|---|
| Activation | Day 0: Activation | |
| TIL | PD-1-selected TIL | |
| Feeders | 100e6 | |
| CM1 | 1000 | mL |
| IL-2 | 6000 | IU/mL |
| OKT3 (30 ng/mL) | 30 | ng/ml |
| G-Rex | 100 | MCS |
| REP | Day 11: REP | |
| TIL | 5e6-200e6 TVC | |
| Feeders | 5e9 | |
| CM2 | 5 | L |
| IL-2 | 3000 | IU/mL |
| OKT3 (30 ng/mL) | 30 | ng/ml |
| G-Rex | 500 | MCS |

| | |
|---|---|
| Split | Day 16: Split |
| | Volume reduce and split up to 5 G-Rex500 |
| | MCS in CM4 + 3000 IU/mL of IL-2 |
| REP Harvest | Day 22: Harvest |
| | REP Harvest |

### Results

Table 39 below specifies the acceptance criteria that was used to evaluate the performance of the small (Extrapolated TVC) and full scale experiment.

TABLE 39
In Process and Harvest Product Release Testing and Acceptance Criteria

| | | |
|---|---|---|
| Test Type | Method | Acceptance Criterion |

In-Process Testing

| | | |
|---|---|---|
| Post-sort Purity (% PD1+) | Flow Cytometry | ≥80% |

Release Testing

| | | |
|---|---|---|
| Appearance | Visual Inspection* | Bag intact, no sign |
| | | of clumps |
| Cell viability | Fluorescence | ≥70% |
| Total Viable Cell Count | Fluorescence | 1 × 10⁹ to |
| | | 150 × 10⁹ |
| Purity (% CD45+ CD3+) | Flow Cytometry | 90% CD45+ CD3+ cells |
| IFNg (Stimulated - | Bead stimulation and ELISA | ≥500 pg/ml |
| Unstimulated) | | |

*Applicable only to full scale experiment.

### Results

Table 40 below were the lists of tumors used in this study and the associated histologies.

TABLE 40
Tumors Used in this Study

| | | |
|---|---|---|
| Experiments | Histology | ID |

PD-1-selected TIL process (intended clinical manufacturing process)

| | | |
|---|---|---|
| Small scale 1 | Ovarian | OV8074 |
| Small scale 2 | Melanoma | M1156 |
| Full scale 1 | Head and Neck | H3046 |

Flow sorting output.

TABLE 41
Pre- and post-sort purity of PD-1-selected TIL by Flow Cytometry.

| | Acceptance | OV8074 | OV8074 | M1156 | M1156 | H3046 | H3046 |
|---|---|---|---|---|---|---|---|
| Parameter | Criterion | (Nivolumab) | (EH12.2.H7) | (Nivolumab) | (EH12.2.H7) | Nivolumab) | EH12.2.H7) |
| Pre-sort % CD3+ (of | N/A | 66 | 62 | 5 | 5 | 49 | 44 |
| FSC/BSC, Singlets) | | | | | | | |
| Pre-sort % PD-1+ (of | N/A | 80 | 70 | 90 | 93 | 14 | 13 |
| CD3+) | | | | | | | |
| Pre-Sort TVC | N/A | 6.3e6 | 2.6e6 | 1.3e7 | 1.3e7 | 1.6e7 | 1.6e7 |
| TVC Sorted | N/A | 3.5e5 | 4.1e5 | 1.1e5 (13%) | 1.1e5 (13%) | 1e5 | 2.4e5 |
| (% Yield) | | (6%) | (16%) | | | (9%) | (23%) |
| Post-sort PD-1⁺ | N/A | 1.24 | 1.4 | 1.07 | 1.08 | 6.8 | 7.4 |
| Enrichment (Fold) | | | | | | | |
| Post-sort Purity % PD-1⁺ (of | ≥80% | 99% | 98% | 96% | 100% | 95% | 96% |
| CD3+) | | | | | | | |

*Purity was based on % PD-1+ (gated on FSC/BSC/CD3)

Post sort purity (% PD-1+) for all three tumors met the criterion of >80%. Activation and REP-Harvest outputs

Table 42 below summarizes the total viable cell count and product attributes from the two small full scale and one full scale experiments, as well as their bulk counterparts (noted in parentheses).

TABLE 42
Summary of the product attributes from Activation and REP

| | | | OV8074 | | M1156 | | H3046 | |
|---|---|---|---|---|---|---|---|---|
| | | Acceptance | Nivolumab | | Nivolumab | EH12.2H7 | Nivolumab | |
| Stages | [symbol] mor ID/Condition | Criterion | staining | 12.2H7 staining | staining | staining | staining | 12.2H7 staining |
| Activation | TVC seeded | N/A | 3.48e5 | 3.48e5 | 1.05e5 | 1.05e5 | [symbol] 2e5 (1.02e5) | 1.02e5 |
| | (Bulk¹) | | (3.48e5) | | (1.05e5) | | | |
| | TVC harvested | N/A | 03e9 (1.14e9) | 1.71e9 | 2.08e8 | 1.50e8 | 1.3e8 | 1.52e8 |
| | (Bulk¹) | | | | (2.19e8) | | (1.37e9) | |
| | Fold expansion³ | N/A | 2960 | 4905 | 1975 | 1427 | 1291 | 1486 |
| | (Bulk) | | (3262) | | (2079) | | (1334) | |
| | # Doublings | N/A | 12 | 12 | 11 | 10 | 10 | 11 |
| | From D0-D11⁴ | | | | | | | |
| REP | TVC seeded | 5−200e6² | 2.00e8 | 2.00e8 | 2.00e8 | 1.50e8 | [symbol] e8 (2.00e8) | 1.52e8 |
| | (Bulk¹) | | (2.00e8) | | (2.00e8) | | | |
| | TVC harvested | N/A | 114.08e9 | 95.94e9 | 86.82e9 | 84.14e9 | 95.2e9 | 80.98e9 |
| | (Bulk¹) | | (99e9) | | (105.00e9) | | (80.81e9) | |
| | % Viability | N/A | 89 | 84 | 97 | 93 | 97 | 97 |
| | Fold expansion³ | N/A | 570 | 480 | 434 | 559 | 720 | 612 |
| | (Bulk) | | (495) | | | | (525) | |
| | # Doublings | N/A | 9.2 | 8.9 | 8.8 | 9.1 | 9.5 | 9.1 |
| | From D11-D22⁴ | | | | | | | |
| | TVC Post-LOVO | 1−150e9 | N/A⁵ | | | | 88.5e9 | N/A⁶ |
| | (% Recovery) | | | | | | (93%)⁷ | |
| | % Viability Post- | >70% | N/A⁵ | | | | 85 | N/A⁶ |
| | LOVO | | | | | | | |
| | % CD45+/CD3+ | >90% | 99.7 | 99.8 | 99.8 | 99.9 | 99.7 | 99.9 |
| | IFNγ (pg/mL) | ≥500 | 948 | 1547 | 4555 | 4371 | 2795 | 3130 |
| | Granzyme B | N/A | 9524 | 9777 | 41603 | 68354 | 33147 | 47603 |
| | (pg/mL) | | | | | | | |
| | % CD4 + CD107A | N/A | 49 | 58 | 34 | 41 | 37 | 38 |
| | (Stimulated) | | | | | | | |
| | % CD8 + CD107A | N/A | 82 | 84 | 85 | 85 | 66 | 67 |
| | (Stimulated) | | | | | | | |

¹Bulk condition TVC shown above are extrapolated to full scale is control for Nivolumab and EH12.2H7
²Range for 5−200e6 TVC seeded at REP based on current established range for Gen 2 REP process, and is not a formal acceptance criterion in this protocol
³Fold expansion = TVC harvested/TVC seeded
⁴Cell doublings was calculated based on the formula “=LOG(Day 22 TVC/Day 11 TVC)/LOG(2)”
⁵Lots were small scale, LOVO was not performed
⁶Single LOVO operation was available. Nivolumab condition was selected for LOVO processing, this represent the clinical manufacturing for PD-1-selected TIL process.
⁷NC-200 cell counter issue was identified during the post-LOVO counting process. Post-thaw recovery count from the stability study (SP-19-003) was used for calculating % Recovery. [symbol]
indicates data missing or illegible when filed

Process Yield: At the end of Activation, TIL selected using either Nivolumab or EH12 staining yielded cell numbers greater than 100e6 (>1200 fold expansion, with an average of 9.1 cell doublings), with sufficient yield to initiate REP culture.

At REP Harvest, all cultures yielded >80e9 TVC. Average of 9 cell doublings were observed between Day 11 to Day 22. The number of cell doublings were very similar to the results observed previous preclinical experiments (TP-19-004R and EXAMPLE 21R).

Dose: From the full scale run (H3046), final product dose using Nivolumab staining was 88.5e9 TVC with 85% viability and 99.7% CD45+CD3+ cells. The final product was a highly enriched TIL product.

Function: Functionality of TIL was characterized based on overnight stimulation of final product with aCD3/aCD28/aCD137 Dynabeads. The supernatants were collected after 24 hours of the stimulation and frozen. ELISAs were performed to assay the concentrations of IFN□ and Granzyme B released into the supernatants. IFN□ release met the acceptance criterion, and all the TIL cultures secreted High levels of Granzyme B upon stimulation. Similar to TIL products generated in prior studies, a high fraction of the TIL from final product expressed CD107A when stimulated with PMA/IO (both CD4+ and CD8+ TIL).

TIL Telomere Length and Telomerase Activity: Data is pending. The report will be amended to include this data when it is available.

TIL Clonality: Data is pending. The report will be amended to include this data when it is available.

Extended Phenotyping: Tables 43, 44, and 45 describe the Extended Phenotype analysis of TIL. Multicolor flow cytometry was used to characterize TIL Purity, identity, memory subset, activation and exhaustion status of REP TIL. <1% of detectable B-cells, Monocytes or NK cells were present in the final harvested TIL (Table 43). REP TIL were consist of mostly by TCRα/β with primarily effector memory differentiation. CD8/CD4 ratio between Nivolumab and EH12.2H7 comparable except for Ovarian tumor. The skewness of CD8/CD4 ratio may be due to heterogenicity of the Ovarian tumor type and lack of selection marker for CD4 and CD8 in the selection procedure.

TABLE 43
TIL Purity, Identity and Memory phenotypic characterization

| | | OV8074 | OV8074 | M1156 | M1156 | H3046 | H3046 |
|---|---|---|---|---|---|---|---|
| Characteristic | | (Nivolumab) | (EH12) | (Nivolumab) | (EH12) | (Nivolumab) | (EH12) |
| Purity | NK cells (CD3− | 0.5 | 0.4 | 0.2 | 0.6 | 0.0 | 0.0 |
| | CD56+) (%) | | | | | | |
| | B cells (CD3− | 0.0 | 0.0 | 0.0 | 0.0 | 0.0 | 0.0 |
| | CD19+) (%) | | | | | | |
| | Monocytes | 0.5 | 0.5 | 0.9 | 0.8 | 0.8 | 0.9 |
| | (CD14+) (%) | | | | | | |
| [symbol] dentity T | TCRα/β (%) | 97.1 | 95.8 | 98.4 | 97.9 | 98.4 | 98.6 |
| cells | TCRγ/δ (%) | 0.1 | 0.3 | 0.0 | 0.0 | 0.0 | 0.1 |
| | TCRα/β+ CD4+ | 19.0 | 40.7 | 19.7 | 10.0 | 64.7 | 62.0 |
| | (%) | | | | | | |
| | TCRα/β+ CD8+ | 80.5 | 57.2 | 79.9 | 89.5 | 34.6 | 37.6 |
| | (%) | | | | | | |
| | TCRα/β+ | 4.2 | 1.4 | 4.1 | 9.0 | 0.5 | 0.6 |
| | CD8/CD4 ratio | | | | | | |
| Memory | Naive: | 0.0 | 0.0 | 0.0 | 0.0 | 0.0 | 0.0 |
| Phenotype- | CCR7+CD45RA+ | | | | | | |
| TCRα/β+ | (%) | | | | | | |
| | T-EM: CCR7− | 98.4 | 98.2 | 98.6 | 98.2 | 98.4 | 97.1 |
| | CD45RA−(%) | | | | | | |
| | T-CM: | 1.4 | 1.8 | 1.4 | 1.7 | 1.6 | 2.8 |
| | CCR7+CD45RA− | | | | | | |
| | (%) | | | | | | |
| | T-EFF/TEMRA: | 0.2 | 0.0 | 0.0 | 0.1 | 0.0 | 0.0 |
| | CCR7− | | | | | | |
| | CD45RA+(%) | | | | | | |

Note:
Gating Algorithm for TIL Purity is shown below:
Monocytes: % Live, CD14+
NK (Natural Killer) Cells: % Live, CD14−, CD3−, CD56+CD16+
B Cells: % Live, CD14−, CD3−, CD19+ [symbol]
indicates data missing or illegible when filed

Due to TCR-stimulated proliferation of TIL, all the PD-1-selected TIL conditions showed upregulation of CD28 expression and downregulation of CD27 expression. In addition, all the PD-1-selected TIL showed less differentiated phenotype with lower KLRG1 expression.

CD27, CD28, CD56, CD57, BTLA, CD25 and CD69 levels were similar to results for Melanoma TIL generated using the Gen 2 manufacturing process.

There is no notable difference between Nivolumab and EH12.2H7 selection procedure in terms of differentiation, activation and exhaustion status.

TABLE 44
Activation and Exhaustion status of CD4+ TIL

| Characteristic | | | | | | | |
|---|---|---|---|---|---|---|---|
| (Gated on Live, CD3+, | | OV8074 | OV8074 | M1156 | M1156 | H3046 | H3046 |
| CD4+) | | (Nivolumab) | (EH12) | (Nivolumab) | (EH12) | (Nivolumab) | (EH12) |
| Differentiation | CD27+ (%) | 5.1 | 6.1 | 5.5 | 12.7 | 34.6 | 39.3 |
| | CD28+ (%) | 99.9 | 99.9 | 99.9 | 99.9 | 100.0 | 100.0 |
| | CD57+ (%) | 27.1 | 16.2 | 65.1 | 37.6 | 13.2 | 14.5 |
| | KLRG1+ | 12.0 | 19.9 | 41.9 | 31.5 | 3.4 | 6.6 |
| | (%) | | | | | | |
| Activation | 2B4+ (%) | 4.1 | 8.7 | 4.8 | 4.8 | 4.0 | 6.1 |
| | BTLA4+ | 99.4 | 99.7 | 99.8 | 99.7 | 99.9 | 100 |
| | (%) | | | | | | |
| | CD25+ (%) | 4.8 | 3.4 | 2.6 | 4.1 | 2.4 | 2.9 |
| | CD69+ (%) | 79.4 | 77.0 | 84.6 | 77.3 | 75.9 | 86.9 |
| | CD95+ (%) | 96.7 | 97.5 | 98.9 | 99.6 | 99.5 | 99.7 |
| | CD103+ | 0.6 | 0.4 | 1.0 | 0.5 | 1.0 | 1.0 |
| | (%) | | | | | | |
| Exhaustion | LAG3+ (%) | 1.9 | 3.0 | 2.7 | 1.4 | 1.6 | 0.9 |
| | PD1+ (%) | 11.8 | 12.0 | 16.5 | 24.1 | 16.2 | 13.9 |
| | TIGIT+ (%) | 15.0 | 24.0 | 33.3 | 51.7 | 31.4 | 37.6 |
| | TIM3+ (%) | 11.1 | 20.7 | 36.4 | 26.3 | 18.4 | 19.8 |

TABLE 45
Activation and Exhaustion status of CD8+ TIL

| Characteristic | | OV8074 | OV8074 | M1156 | M1156 | H3046 | H3046 |
|---|---|---|---|---|---|---|---|
| (Gated on Live, CD3+, CD8+) | | (Nivolumab) | (EH12) | (Nivolumab) | (EH12) | (Nivolumab) | (EH12) |
| Differentiation | D27+ (%) | 10.3 | 8.7 | 27.2 | 24.5 | 23.1 | 28.1 |
| | D28+ (%) | 99.9 | 99.8 | 99.9 | 99.9 | 99.8 | 99.9 |
| | D57+ (%) | 39 | 17.7 | 52.8 | 38.7 | 15.5 | 11.8 |
| | LRG1+ (%) | 36.9 | 23.7 | 15.5 | 9.9 | 4.1 | 6.1 |
| Activation | [symbol] 34+ (%) | 3.2 | 3.5 | 2.3 | 2.1 | 4.0 | 4.0 |
| | TLA4+ (%) | 99.7 | 99.8 | 99.8 | 99.8 | 99.9 | 99.9 |
| | D25+ (%) | 0.5 | 0.9 | 0.2 | 0.4 | 0.6 | 0.5 |
| | D69+ (%) | 76.1 | 74.4 | 81.6 | 84.4 | 86.2 | 87.2 |
| | D95+ (%) | 95.3 | 88.7 | 98.9 | 98.5 | 96.5 | 96.6 |
| | D103+ (%) | 0.5 | 0.5 | 0.8 | 0.3 | 0.9 | 0.6 |
| Exhaustion | AG3+ (%) | 0.6 | 1.8 | 1.9 | 0.9 | 1,5 | 0.9 |
| | D1+ (%) | 12.1 | 4.7 | 11.7 | 14.6 | 7.6 | 10 |
| | [symbol] IGIT+ (%) | 51.6 | 42.7 | 78.7 | 85.5 | 17 | 19.8 |
| | [symbol] IM3+ (%) | 15.3 | 22.1 | 27.3 | 49.1 | 21 | 16.2 |

[symbol]

indicates data missing or illegible when filed

Additional analysis on the phenotypic characterization data to establish the equivalence of EH12.2H7 and Nivolumab.

PD-1-selected TIL generated using EH12.2H7 and nivolumab to obtain PD-1⁺ TIL were assessed for the expression of CD4, CD8, CCR7, CD45RA, and PD-1 by flow cytometry. No significant differences were observed in expression of CD4 and CD8 in PD-1-selected derived using nivolumab and EH12.2H7. For the three assayed tumors, both TIL products yielded a higher proportion of CD8⁺ T cells relative to CD4⁺ T cells (FIG. 1). The similarity in CD4 and CD8 expression in the three PD-1-selected TIL products suggests that selecting for PD-1+ using nivolumab did not alter the ratio of CD4/CD8 compared to EH12.2H7.

Like T cell lineage, the memory status of the TIL was similar in the PD-1-selected TIL generated using EH12.2H7 and nivolumab. The TIL populations were composed predominantly of effector memory T cells PD-1-selected TIL generated using nivolumab and EH12.2H7 resemble Iovance's LN-145 investigational product, suggesting that selecting for PD-1 using either anti-PD-1 clone does not skew the memory phenotype of the TIL.

To assess whether PD-1 expression was similarly reduced upon culture, PD-1-selected TIL generated using nivolumab and EH12.2H7 were assessed pre- and post-expansion. Post-sort, percentages of PD-1+ TIL were close to 100% in both freshly sorted TIL preparations (Tables above). PD-1 expression was significantly and comparably reduced post-expansion in PD-1-selected generated using EH12.2H7 and nivolumab. As predicted, the reduction in PD-1 expression upon expansion suggests that the previously high PD-1 expressors in the PD-1+ sorted TIL using EH12.2H7 and nivolumab reverted to mostly PD-1− with expansion.

Functional Characterization of PD-1-selected TIL generated from EH12.2H7 and Nivolumab-sorted PD-1+ TIL

To assess whether expanded PD-1+ TIL derived using nivolumab were similarly functional to TIL derived using the EH12.2H7 clone, PD-1-selected TIL from 3 tumors were stimulated non-specifically with αCD3/αCD28/α41BB activation beads and evaluated for IFNγ and Granzyme B secretion. Nivolumab and EH12.2H7-derived PD-1-selected TIL produced similar levels of IFNγ and Granzyme B in response to stimulation. PD-1-selected TIL generated using nivolumab and EH12.2H7 secreted appreciable levels of IFNγ and Granzyme B in response to a non-specific stimulation (αCD3/αCD28/αCD137 beads), suggesting that the selected TIL were highly functional post-expansion.

### Information

On Day 0, due to logistic issues fresh tumor could not be received for the example. All the experiments were executed using frozen Tumor digest in lieu of fresh tumor. Data from research study suggest that there is no difference in PD-1 expression when fresh or frozen tumor was tested.

### Conclusions and Recommendations

PD-1-selected TIL process was developed at full scale to expand PD-1+ TIL to >80 e9 in 22 days. All six lots (Both Nivolumab and EH12 staining method, 2 full scale and 4 small scale) manufactured at development scale met the acceptance criteria for release parameters.

TABLE 46
Summary Table:

| | Acceptance | OV8074 | OV8074 | M1156 | M1156 | H3046 | HI3046 |
|---|---|---|---|---|---|---|---|
| [symbol] esting Parameters | Criterion | (Nivolumab) | (EH12) | (Nivolumab) | (EH12) | (Nivolumab) | [symbol] EH12) |
| [symbol] ppearance | Bag intact, no | NA | NA | NA | NA | Pass | Pass |
| | [symbol] ign of clumps | | | | | | |
| [symbol] ell viability | ≥70% | Pass | Pass | Pass | Pass | Pass | Pass |
| [symbol] otal Viable Cell | ×10e9 to 150 | Pass | Pass | Pass | Pass | Pass | Pass |
| [symbol] ount | × 10e9 | | | | | | |
| [symbol] entity | >90% | Pass | Pass | Pass | Pass | Pass | Pass |
| [symbol] CD45+/CD3+) | CD45+CD3+ | | | | | | |
| | cells | | | | | | |
| [symbol] Nγ(Stimulated - | ≥500 pg/ml | Pass | Pass | Pass | Pass | Pass | Pass |
| [symbol] nstimulated) | | | | | | | |

NA, Not applicable, cells were harvested in small scale [symbol]
indicates data missing or illegible when filed

Overall, this Example demonstrated that PD-1-selected TIL generated from PD-1-sorted TIL using nivolumab were comparable to TIL generated using the EH12.2H7 clone, thereby supporting the use of nivolumab for PD-1 selection in the clinical manufacturing.

### Example 6: Selection of PD-1⁺ TIL Using Nivolumab by Flow Cytometry Sorting and Expansion in Full-Scale for Clinical Manufacturing

### Preparation of CM1 Media for Day 0 and CM2 Media for Day 11

Information: One batch of CM1 was 10 L.

Information regarding IL-2. Chose the IL-2 following the priority order and then availability. Completed the calculations accordingly.

First: IL-2 Akron prefilled syringe 1 mL ready to use. Calculated Volume of IL-2 needed to prepare one 10 L bag of CM1 at 6000 IU/mL: (10 000 mL×6000 IU/mL=60×10⁶ IU per bag). IL-2 to transfer: 60×10⁶ UI/IL-2 Specific Activity=______mL of IL-2 per 10 L RPMI bag (1 decimal). Calculated Number of syringes needed: Total volume of IL-2/volume per syringe=______mL/1 mL=______syringe (rounded up at 0 decimal).

Second: IL-2 Akron powder to reconstitute with 1 mL WFI. Calculated the mg of IL-2 needed to prepare one 10 L bag of CM1 at 6000 IU/mL: (10000 mL×6000 IU/mL=60×10⁶ IU per bag). 60×10⁶ IU/IL-2 specific activity convert to mg of IL-2 per 10 L RPMI bag (1 decimal). IL-2 to transfer (1 mg=1 mL) convert to mL of IL-2 per 10 L RPMI bag (1 decimal). Calculated the Total number of mg needed of IL-2. Calculated the number of bags and vials needed.

Last: IL-2 Cellgenix powder to reconstitute with 2 mL Acetic acid. Calculated the mg of Il-2 needed to prepare one 10 L bag of CM1 at 6000 IU/mL: (10000 mL×6000 IU/mL=60×106 IU per bag). 60×10⁶ UI/IL-2 Specific Activity convert to mg of IL-2 per 10 L RPMI bag (1 decimal). IL-2 to transfer; calculated the total mg needed of IL-2. Calculate mg of IL-2 per bag and number of bags.

Thawing of human AB serum: 10 bottles×100 mL human AB Serum per 10 L of CM1 to prepare.

### Preliminary Preparation

IL-2 to use from section 1: If Akron IL-2 in prefilled syringe, no Reconstitution was required. If Akron IL-2 in powder, went to step 2.2 proceed with reconstitution. If Cellgenix IL-2 in powder, went to step 2.5 proceed with reconstitution. Recorded the number of vials to reconstitute. Transferred materials into the BSC:

Spiked Water for Injection (WFI) bottle using a 10 mL syringe, drew 1 mL of WFI. Connect an 18 G needle to the syringe and transferred 1 mL WFI to vial of IL-2. Inverted the vials 2-3 times and swirled until all powder was dissolved. Avoided foam formation and did not mix vigorously. Repeated this step to reconstitute the needed number of vials (use a new syringe if needed). Kept in BSC until use. Recorded the number of vials to reconstitute. Transferred materials into the BSC.

Opened HAc bottle, using a 10 mL syringe with 18 G needle connected or pumpmatic pipette, drew 2 mL of HAc. Transferred 2 mL HAc into the IL-2 vial through the septum. Inverted the vials 2-3 times and swirled until all powder was dissolved. Avoid foam formation and did not mix vigorously. Repeated this step to reconstitute the needed number of vials in section 5 Keep in BSC until use,

Prepared CM1 media and labeled. When using two 5 L RPMI+ GlutaMAX bags, transferred the 10 L Labtainer labelled CM1 Pool Media into the BSC and attached an extension set. Attached one end of the pump boot to CM1 Pool Media and the other end to the first 5 L RPMI+ GlutaMAX bag. Pumped the entire volume into CM1 Pool Media. Repeated with second bag of 5 L RPM+ GlutaMAX so that total volume of RPMI+ GlutaMAX in CM1 Pool Media was 10 L

Pipetted 10 mL of 2-mercaptoethanol into the tube 2-mercaptoethanol. Added 10 mL of Gentamicin into one bottle of human AB Serum and homogenize. Marked bottle to indicate addition. Dew the needed volume of IL-2 for a 10 L bag (see section 5) and transferred into one bottle of human AB serum and homogenized. Placed the pipette tip into 2-mercaptoethanol and aspirated the 10 mL into CM1 Pool Media.

Aspirated the 100 mL human AB serum bottle with IL-2 added into CM1 Pool Media. Aspirated the 100 mL human AB serum bottle with gentamicin added into CM1 Pool Media. Aspirated the remaining 8 bottles of human AB serum into CM1 Pool Media.

Placed CM1 Media on the balance and tare. Pump 990±10 mL from CM1 Pool Media into CM1 Media. Assumed 1 g was equal to 1 mL. Recorded the volume CM1 Media in step 12.20. Heat sealed and removed CM1 Media. Repeated with remaining CM1 Media bags. Stored bags at 2-8° C.

Heat sealed and removed CM1 Pool Media from the pump boot and retained for sterility testing. Removed 20 mL of media from Sterility CM1 bag. Inoculated 10 mL into an anaerobic BacT/Alert bottle and 10 mL into an aerobic BacT/Alert bottle. Bag could be discarded after sampling. If sending out for testing: Placed the Sterility CM1 bag at 2-8° C. Sterility was done by sending sample to an outside vendor for sterility by membrane filtration. Post-processing for applicable microbiology cultures and record accession numbers.

### IL-2 Proleukin Aliquots Preparation

Preparation of 1% HAD in PlasmaLyte A

Wiped the outside of all reagents and supplies with 70% Isopropanol alcohol and placed in BSC.

Added 16 mL of 25% I-ISA stock solution to 384 mL of PlasmaLyte A into in a sterile filter unit. Recorded volumes below. Note: The above volume was enough to prepare one IL-2 vial at final concentration of 6×10⁴ IU/mL.

Filtered the media through a 0.22 gm filter unit.

Labeled as 1% FBA in PlasmaLyte A.

Preparation of rhIL-2 stock solution.

Prepared rhIL-2 stock solution (6×10⁸ IU/mL final concentration) in 1% HSA in PlasmaLyte A.

Attached an 18 G needle to a 3 mL syringe and draw up 1.2 mL of WFI. Injected into vial of IL-2. Did not remove syringe from vial.

Inverted the vial 2-3 times and swirled until all powder was dissolved. Did not shake or vortex to prevent foaming. Without removing the syringe from the vial, drew out and measured (recorded as A in the table in step 2.4) the solution from the vial and placed in a 500 mL sterile bottle.

Calculated volume of 1% HSA diluent required. Note: per manufacturer instructions after reconstituting with 1.2 mL of WFI, each vial contained 18×10⁶ IU/mL.

Labeled the 500 mL sterile bottle IL-2 working stock 6×104 IU/mL. Transferred the calculated amount of 1% HSA (D from step 2.4) into the 500 mL sterile bottle to which the reconstituted IL-2 was already added. Mix well. Transferred an appropriate amount to a sterile specimen cup if necessary for ease of aliquoting. Labeled as IL-2 working stock 6×104 IU/mL.

Aliquoted the reconstituted IL-2 from 1L-2 working stock 6×10⁴ IU/mL in 1 mL aliquots into labeled tubes.
Labeled the tubes as Proleukin 1L-2, 6×10⁴ IU/mL
Recorded preparation date, lot #, expiration, volume, and operator initials.
Stored at −80° C.
Expired 3 months after preparation

After aliquoting was complete, recorded the number of 1 mL aliquots prepared.

### PD-1 SELECTED TIL Process Day 0

Recorded start date and time of CM1 media incubation. Incubation of CM1 media bag(s) overnight prior to processing. Recorded date of most recent Gating and Compensation performed on the Sony Cell Sorter. Verified that the Sony FX500H Cell Sorter had been turned on and or operated in the last 30 days.

Preparation of tumor wash medium, sorting buffer, and collection buffer. Labeled the empty 500 mL bottle as Sorting Buffer. Clamped the plasma transfer set and used the spike to spike the PBS/EDTA bag. Using a syringe, transferred 490 mL of PBS/EDTA to Sorting Buffer bottle. Added 10 mL of FBS to 490 mL of PBS/EDTA. Store at 2-8° C. when not in use.

Labeled one of the 500 mL bottles of HBSS as Tumor Wash Medium. Added 5 mL of gentamicin (50 mg/mL) to the 500 mL bottle of HBSS labeled Tumor Wash Medium.

Labeled 15 mL conical tube Collection Buffer. Added 7 mL of HBSS and 7 mL of Human AB Serum to the 15 mL conical tube. Stored at 2-8° C. when not in use.

Reconstitution of enzymes: Collagenase AF-1 (1), Neutral Protease (1), DNase I (2). Reconstituted the reagents in the following steps, if applicable, and stored at 2-8° C. when not in use. If aliquots were prepared in advance, N/A applicable step(s).

If applicable, reconstituted the lyophilized vial of Collagenase AF-1 (Nordmark, Sweden, N0003554) in 10 mL of sterile HBSS using an appropriately sized syringe and needle. The lyophilized stock enzyme was at a concentration of 2892 PZ U/vial. After reconstitution the collagenase stock was 289.2 PZ U/ml.

If applicable, reconstituted the Neutral protease (Nordmark, Sweden, N0003553) in 1 mL of sterile HBSS using an appropriately sized syringe and needle. The lyophilized stock enzyme was at a concentration of 175 DMC U/vial. The lyophilized stock enzyme may be at a concentration of 175 DMC/mL.

Reconstituted the DNAse I (Roche, Switzerland, 03724751) in 1 mL of sterile HBSS using an appropriately sized syringe and needle. The lyophilized stock enzyme was at a concentration of 4 KU/vial. After reconstitution the DNAse I stock was 4 KU/mL. Prepared two vials.

Preparation for tissue dissection. Labeled 4 wells of the ‘Fragments for C-Tubes 1’ plate ‘1’, ‘2’, ‘3’, and ‘4’. Labeled the remaining wells of the ‘Fragments for C-Tubes 1’ plate with an Labeled 4 wells of the ‘Fragments for C-Tubes 2’ plate ‘5’, ‘6’, ‘7’, and ‘8’. Added 5 mL of ‘Tumor Wash Medium’ into all labelled wells of the 6-well plates labelled ‘Fragments for C-Tubes 1’ and ‘Fragments for C-Tubes 2’ Added 50 mL of Tumor Wash Medium to each 100 mm petri dishes labelled ‘Wash 01’, ‘Wash_02’, ‘Wash 03’ and ‘Unfavorable’. Added 20 mL of Tumor Wash Medium into each of 50 mL conical tubes labelled ‘Forceps Wash Medium’, ‘Scalpel Wash Medium’. Keep the tumor wash medium in the BSC for further use. For dissection only, placed scalpels and forceps in appropriate tubes labelled ‘Forceps Wash Medium’, ‘Scalpel Wash Medium’.

### Tissue Dissection

Transferred the tumor container into the BSC. Use long forceps, transferred the tumor(s) from specimen bottle to 100 mm petri dish labelled Wash_01′. Incubated the tumor at ambient temperature in Wash_01′ for 3 min. Recorded time. Recapped specimen bottle and transfer to balance. Recorded weight of specimen bottle and calculated weight difference of tumor tissue. Transferred the following into the BSC: 10 mL serological pipette; 50 mL conical tube labelled “Tumor shipping medium”. Transferred of 10 mL of tumor shipping medium into the tube labelled “Tumor shipping medium”. Drew 10 mL of the Tumor Shipping Medium into a syringe with an 18 G needle. Inoculated one each anaerobic and aerobic sterility bottle with 5 mL of tumor shipping medium. Recorded incubation stop time of tumor (after incubation 3 min) Using long forceps, transferred tumor to 100 mm Petri dish labelled ‘Wash_02’ and incubated tumor at ambient temperature for 3 min. Recorded incubation stop time Using long forceps transfer tumor to 100 mm petri dish labelled ‘Wash_03’ and incubate tumor at ambient for 3 min. Recorded incubation stop time of tumor. Placed the ruler underneath the lid of a 100 mm petri dish (dish lid) and used the long forceps to transfer the tumor to the lid for measurement and dissection. Measured and recorded length of the tumor and the number of fragments received. The length of the tumor was measured as the sum of all individual fragment lengths.

Performed an initial dissection of the tumor on the dish lid. Dissected into three intermediate pieces, or group into 3 groups of equivalent volume. While cutting took care to conserve the tumor structure of each intermediate piece. Transferred any intermediate tumor pieces not being actively dissected into separate ‘H’ wells of the ‘Fragments for C-Tubes 1’ 6-well plate to keep the tissue hydrated.

Dissection Start: the dissection target time of each intermediate fragment was within 20 min, on average. Started the final fragmentation for first intermediate fragment. Recorded dissection start time of the first intermediate fragment.

Gently dissected the tumor into approximately 216 mm3 fragments (6×6×6 mm), using the ruler under the 100 mm petri dish lid as a reference. Worked quickly and dissected the entire tissue into fragments. Using transfer pipette, scalpel, or forceps selected up to four tissue fragments for culture and transferred the fragments to numbered well of the ‘Fragments for C-Tubes’ 6 well plate. Filled each well with 4 fragments before adding fragments to another well. Each well represents a C-Tube that was used in the digest. Took care to always keep the tissue hydrated throughout the dissection procedure. Transferred the unfavorable tissue and waste into the ‘Unfavorable Tissue’ dish. Unfavorable tissue was indicated by yellow adipose tissue or necrotic tissue. Used maximum of 3 wells of the 6 well plate per intermediate fragment. If necessary, used wells of second 6 well plate to accommodate up to 8 C-tubes. Fresh scalpel or forceps was used, according to discretion of operator.

Recorded dissection stop time all fragments. Counted total fragments dissected from the three intermediate fragments. Each well that was used should have had 4 fragments. If extra fragments existed, added 1 extra fragment per well for a maximum of 5 fragments. 4 fragments per C-tube was optional; 3-5 fragments were allowed depending on number of total fragments available. Recorded the final number of fragments in wells 1 through 8. Stored the fragments in the six-well plates until needed to ensure tissue stays hydrated. Each well represented one C-tube. Prepared up to 8 C-tube for the Octodissociator. If more than 40 total fragments, retained excess in a 50 mL conical labeled with patient identifiers and an appropriate amount of Tumor Wash Medium to ensure tissue stays hydrated. Notified Iovance of available excess tumor. Excess tumor was discarded after 48 hours.

### Preparation of Tumor Digest Solvent

For each C-Tube, labeled as ‘Tumor Digest accompanied by a number starting with the first tube and added the following volumes:
4.7 mL of sterile HBSS
10.2 pL of Neutral Protease
21.3 pL of Collagenase AF-1
250 pL of DNAse 1
Record the number of C-tubes prepared.

For each well containing fragments to be digested used forceps to transfer all tumor fragments to a corresponding C-Tube labelled as instructed in step 6.2. (1)

Tumor Digest

Inserted the C-Tube (“Tumor Digest 1”, “Tumor Digest 2”, “Tumor Digest 3”, etc.) into the bracket on the GentleMACS OctoDissociator.

Recorded type listed. Set GEntleMACS OctoDissociateor to appropriate program based upon the list of tumor tissue types and mark which program was selected. Table 47, below.

| Tumor Tissue Type | Designation |
|---|---|
| Melanoma, Ovarian, Colon, Hypopharyngeal, and Renal | Soft |
| Lung (NSCLC), Prostate and Colorectal | Medium |
| carcinoma | |
| Breast, Pancreatic, Hepatocellular, | Tough |
| Head and Neck Squamous | |
| Cell (HNSCC), TNBC | |

Started the OctoDissociator. Recorded digestion start time. Removed C-Tubes. Recorded digestion stop time.

### Post Tumor Digestion Treatment

Positioned a 70 pm cell strainer on the Post Digest 1 tube and used a 25 mL serological pipette to transfer the contents of the Tumor Digest through the filter and into the ‘Post Digest 1’ tube. Repeated for up to 4 ‘Tumor Digest’ tubes. Changed strainer as needed or for each C-tube. If >4 Tumor Digest C-Tubes, used a 70 pm cell strainer and filtered each remaining C-Tube into the labelled Post Digest 2 tube. Changed the strainer as needed between C-Tubes.

Using a serological pipette, gently rinsed the inside of each Tumor Digest C-Tube with 5 mL of HBSS. Inverted the C-tube to thoroughly rinse and filtered the 5 mL into the corresponding Post Digest tube through the 70 pm cell strainer. Discarded cell strainer after all tubes were rinsed.

Using a serological pipette, added HBSS to the Post Digest tube(s) up to the 50 mL mark. Transferred “Post Digest” tube(s) to the centrifuge and centrifuged at 400×g for 5 mins at RT with full acceleration and braked.

Removed one bag of CM1 from the incubator. Using a syringe, collected approximately 30 mL of CM1 and placed in a 50 mL conical labeled ‘CM1’. Transferred 450 pL of CM1 into each of the cryovials labelled C1-C4.

Transferred the Post Digest tube(s) to the BSC. Gently aspirated supernatant and discarded. Use 5 mL serological pipettes to resuspend cell pellet(s) in 5 mL of warm CM1. Pipetted up and down 6 times to resuspend the cell pellet(s). If there was a “Post Digest 2” tube, transferred the contents to the “Post Digest 1” tube. Measured and recorded the volume of the “Post Digest 1” tube.

Immediately after resuspending the pellet, transferred 50 pL to “C1”, “C2”, “C3”, and “C4”. Pipetted up and down 3 times to wet the tip before taking the sample.

Used ‘Viability and Cell Count Iovance’ protocol on the NC-200. Using the NC-200, performed a cell count on sample 1. Samples were prepared at a 1:10 dilution. If necessary, prepared an additional appropriate dilution. Recorded dilution factor used. Recorded the viable (live) cell concentration and viability as needed. Repeated for samples 2, 3, and 4. Recorded information.

Calculated the average of the four counts using the data recorded in step 8.9 (Post Digest 1+ Post Digest 2+ Post Digest 3+ Post Digest 4)/4

Calculated the number of total viable cells. [Volume of cell suspension−0.2 mL for counts]×average concentration.

### Tumor Digest Staining

Calculated the volume of 5×10⁵ viable cells. Transfer that volume from the Post Digest tube to the PE FMO Prep and FITC FMO Prep tubes. Place at 2-8° C. when not in use.

Calculated the TVC remaining in Post Digest tube.

Added 10 mL of HBSS to the PE FMO Prep and FITC FMO Prep tubes. Added 5 mL of HBSS to the Post Digest tube.

Transferred all tubes to the centrifuge. Centrifuged at 400×g for 5 mins at RT with full acceleration and full brake.

Diluted the concentrated Nivolumab solution [10 mg/mL] by performing a 1:100 dilution as follows: Added 10 pL of Nivolumab to 990 pL of Sorting Buffer in a cryovial and vortexed gently for 5 seconds to mix thoroughly. Placed at 2-8° C. until further use.

Anti-IgG4-PE: Diluted anti-IgG4-PE solution [0.5 mg/mL] by performing a 1:50 dilution as follows: Added 10 pL of anti-IgG4-PE to 490 pL of Sorting Buffer in a cryovial and vortexed gently for 5 seconds to mix thoroughly. Placed at 2-8° C. until further use.

Transferred Post Digest, PE FMO Prep, and FITC FMO Prep tubes back to the BSC. Aspirated and discarded the supernatants from each tube and resuspended as follows:

Post Digest resuspended cells at 10×10⁶ cells/mL by calculating as follows: (TVC from step 9.3)÷10×106 cells/mL=Sorting Buffer to add (mL) Agitated the pellet and then added the calculated volume of Sorting buffer (Rounded up to the next mL). Pipetted up and down 5 times.______(TVC) cells/10×10⁶ cells/mL=mL (1 decimal).

FITC FMO Prep resuspended by agitating the pellet and adding 300 pL of Sorting buffer with a micropipette. Pipetted up and down 3 times

PE FMO Prep resuspend by agitating the pellet and adding 300 pL of Sorting buffer with a micropipette. Pipette up and down 3 times. Stored PE FMO Prep at 2-8° C. until finished with the 30-minute Nivolumab incubation.

For every 1 mL of sorting buffer added (step 9.9) to Post Digest added 10 uL of the 1:100 diluted Nivolumab solution. Added 10 uL of the 1:100 diluted Nivolumab solution to FITC FMO Prep.

Mixed both tubes gently by flicking and incubate cells at 2-8° C. for 30 minutes. Agitated by flicking gently every 10 minutes during incubation to ensure thorough staining. Checked a box below after each periodic agitation.

After incubation, added 10 mL of Sorting Buffer to the Post Digest and FITC FMO Prep RT with full acceleration and full brake.

Transferred the Post Digest and FITC FMO Prep tubes back to the BSC. Gently aspirated supernatant and discard. Resuspended cells as follows was steps below.

Post Digest: resuspended by agitating the pellet and adding 400 pL of Sorting buffer with a micropipette. Pipetted up and down 3 times.

FITC FMO Prep: resuspended by agitating the pellet adding 300 pL of Sorting buffer with a micropipette. Pipetted up and down 3 times.

Measured the volumes of the Post Digest and FITC FMO Prep tubes using 1 mL serological pipettes with a pipette aid; recorded the volumes below: Volume of Post Digest. Volume of FITC FMO Prep.

To the Post Digest and FITC FMO Prep, added 10 pL of intermediate diluted anti-IgG4-PE per 0.1 mL of volume according to the calculations below in steps 10.22 and 10.23. Then placed ‘FITC FMO Prep’ at 2-8° C.

Calculation: 10 uL of intermediate diluted anti-IgG4-PE×(Post Digest volume/0.1 mL)

Calculation: 10 uL of intermediate diluted anti-IgG4-PE x (“FITC FMO Prep” volume/0.1 mL\Transferred PE FMO Prep to the BSC. Measured the volume of PE FMO Prep. To the Post Digest and PE FMO Prep, added 3 pL of anti-CD3-FITC per 0.1 mL of volume according to the calculations.

Calculation: 3 uL anti-CD3-FITC (PE FMO Prep volume/0.1 mL).

Calculation: 3 uL anti-CD3-FITC (PE FMO Prep volume/0.1 mL).

Mixed Post Digest, FITC FMO Prep, and PE FMO Prep tubes by agitating gently and incubated cells at 2-8° C. for 30 minutes. Agitated by flicking gently every 10 minutes during incubation to ensure thorough staining.

After incubation, added 10 mL of Sorting Buffer to the “Post Digest”, “PE FMO Prep”, and “FITC FMO Prep” tubes.

Filtered each tube through 30 uM Pre-Separation Filters into labeled 15 mL conical tubes Post Digest Sort, PE FMO, and FITC FMO respectively.

Centrifuged Post Digest Sort, PE FMO, and FITC FMO tubes at 400×g for 5 mins at RT with full acceleration and full brake.

Transferred Post Digest Sort, PE FMO, and FITC FMO back to the BSC. Aspirated and discarded the supernatants from each tube and resuspended gently in residual supernatant. Resuspended further as follows.

Post Digest Sort: resuspended cells at 510×106 cells/mL by agitating the pellet and adding the volume of Sorting Buffer calculated. Used the initial volume; Did not round up to the next mL. Pipetted up and down 3 times gently to mix thoroughly. Placed covered tube in the dark at 2-8° C. until ready to sort.

PE FMO: resuspended by agitating the pellet and adding 300 pL of Sorting Buffer with times. Placed covered tube in the dark at 2-8° C. until ready to sort.

FITC FMO: resuspended by agitating the pellet and adding 300 pL of Sorting Buffer times. Placed covered tube in the dark at 2-8° C. until ready to sort.

Added 2 mL Collection Buffer to a 15 mL conical tube labelled PD-1 Positive; repeated for a PD-1 Negative tube.

Initiated Flow cytometry Sorting of PD-1-selected TIL from Tumor Digest. If the PD-1 positive tube acquired more than 1×106 cells, sorting may be stopped. The maximum number of PD-1 selected TIL to seed the G-Rex 100MCS flask with was 1×106±10% cells. If the PD-1 negative tube acquired more than 4×106 sorted cells, replaced that tube with another 15 mL conical tube containing 2 mL of Collection Buffer and added an number to the label as a suffix to distinguish multiple tubes. Always stored sorted cells at 2-8° C. until ready to place into G-Rex 100MCS Flask. Recorded total number of PD-1 selected TIL collected post-sort.

Prepared G-Rex100MCS flask with CM1

Removed CM1 media bag from incubator and recorded date and time. Ensured media was warmed overnight.

Closed all clamps of the G-Rex100MCS. Did not close the clamp of the filter line on the G-Rex100MCS.

Welded the CM1 media bag to the red line of the G-Rex100MCS.

Placed the G-Rex100MCS on a scale and tare. Transferred by gravity 400 t 10 mL of CM1 into the G-Rex100MCS. Recorded volume transferred. Considered 1 g was equal to 1 ml.

Heat sealed off red line of the G-Rex100MCS.

Labeled G-Rex100MCS PD-1-selected TIL DO and transferred the flask and CM1 media bag to the 37° C. incubator until further use.

Identification of G-Rex 100MCS “PD-1-selected TIL DO”

Prepared feeder bag. Sterile welded the CM1 bag to the Feeder Cells bag. Placed Feeder Cells bag on the balance and tare. Transferred by gravity 100 mL±10 mL of CM1 into the Feeder Cells bag and recorded the volume added. Considered 1 g was equal to *1 ml. Heat sealed and removed feeder bag from the extension set leaving the same original length of tubing.

Prepared feeder cells. Recorded lot number of feeder cell bag. Recorded the temperature of water bath before feeder thawing. Thawed feeder cell bag for 3-5 min in a 37° C. water bath until only small ice chunks remained. Recorded start time of thawing; end time of thawing. Removed feeder cell bag from water bath and verify bag was dry.

Connected the Feeder Cells bag to the harness using a Luer connector or by sterile welding. Replaced syringe harness with a new 50 mL syringe. Spiked the thawed feeder cell bag with a spike from the harness into the single port of the thawed feeder bag. Rotated the stopcock valve so the Feeder Cells bag was in ‘OFF’ position. The valve indicated what was closed. Opened the clamps to the thawed feeder bag line approximately 10 mL of feeders from the feeder cell bag into the syringe with a single draw. Rotated the stopcock valve so the thawed feeder cells bag was in the ‘OFF’ position and opened all clamps in direction of the Feeder Cells bag. Dispensed the contents of the syringe into the Feeder Cells bag while gently mixing. If necessary, drew air back from the bag and use to completely clear the line. Rotated the stopcock so the Feeder Cells' bag was in ‘OFF’ position. Mixed the cells in the ‘Feeder Cells’ bag well. Attached a 10 mL syringe to NIS port of the ‘Feeders’ bag, mixed the bag and remove a 1 mL sample through the NIS port. Transferred sample into cryovial 1. Repeated this for cryovials 2-4 using a new syringe for each sample.

### Calculated the Volume of Feeder Cells

Feeder cell count. Prepared appropriate dilution (1:10 was recommended) Using the NC-200, performed a cell count on sample 1 Recorded dilution factor used. Recorded the viable (live) cell concentration and viability below. Repeated for samples 2, 3, and 4. Calculated the average viable cell concentrations of the four counts using the data recorded in step 13.2 (Feeder Cells 1+Feeder Cells 2+Feeder Cells 3/Feeder Cells 4)/4. Calculated the number of total viable feeder cells. If total viable cell number was more than 1×10⁸ cells, continued to section 14.

Addition of feeder cells to G-Rex100MCS. Calculated volume of feeders for 100×106 cells. If a large volume of feeder cells must be removed, a second syringe can be used to complete the volume reduction and clear the line. Determined the volume to remove. Removed the calculated volume (step 14.3) from the feeder bag using an appropriately sized syringe. Repeated as necessary using a fresh syringe for each removal of volume from the bag. Using a new syringe, drew up an appropriate amount of air and dispensed the air to clear the line. Discarded the volume of removed cells. Record volume removed. Using a 1 mL syringe with 18 G needle attached, drew up 0.030 mL of OKT3. Remove needle and dispensed OKT3 into the ‘Feeder Cells’ by the NIS. Flushed the syringe with at least 0.5 mL of feeder cell suspension to ensure all OKT3 was added into the bag. Ensured enough air was in Feeder Cells to allow gravity feed into the G-Rex 100MCS. If necessary, drew a sufficient volume of air into a new syringe and added to Feeder Cells through the NIS to clear the line. Heat sealed off the ‘Feeders’ bag, leaving enough tubing for future welding.

Removed the G-Rex100MCS PD-1-selected TIL DO from incubator and place besides the welder. Sterile welded the ‘Feeder Cells’ bag, using one of the unused tubings to the red line on the G-Rex100MCS.

Unclamped the line and allowed the feeder cells to flow into the G-Rex 100MCS by gravity. Closed the clamps and heat sealed off the ‘Feeder Cells’ bag close to the original weld.

Adding PD-1 selected TIL into G-Rex100MCS and final CM1 Media addition

Sterile welded a 4″ plasma transfer set or extension set to the red line of the GREX 100 MCS to add a usable luer connection to the red line. Transferred the G-Rex 100 MCS to the BSC.

Capped PD-1 Positive tube and invert gently 5-10 times. Removed cap and used the pumpmatic pipette and aspirated the contents of the PD-1 Positive tube. Repeated if there were additional PD-1 Positive tubes. Recorded the volume. Inverted the pumpmatic pipette and drew 2 mL of air into the syringe.

Disconnected the pumpmatic pipette from the syringe. Connected to the red line of the G-Rex 100MCS. Dispensed the volume in the syringe into the red line and used the air in the syringe to dear the line. Used a new syringe with more air if necessary to clear the line. Heat sealed the red line of the G-Rex 100MCS.

Calculated volume necessary to add to G-Rex 100MCS to bring final volume to 1000 mL.

Sterile welded the CM1 bag to the red line of the G-Rex 100MCS. Placed the G-Rex 100MCS on the scale and tare. Allowed CM1 to flow by gravity into the flask until the target volume (step 15.5+10 mL) was reached. Recorded volume added. Heat sealed red line of flask. Note: Considered 1 g was equal to 1 mL.

Returned flask to the BSC. Record time. Waited 20 minutes for the TIL to settle and then connect one 10 mL syringe to the blue capped NIS and drew 2 mL of media. Recorded volume drawn. Inoculated one anaerobic BacT/Alert bottle and one aerobic BacT/Alert bottle each with 1 mL of the flask supernatant. Recorded time.

Placed G-Rex 100MCS in the incubator. Recorded stop time of processing.

Verified and recorded incubation conditions and recorded incubator ID.

### Cell Count Results

TABLE 48
C14503-1010-101/FITC FMO (Data Source −1)

| Name | Events | % Parent | % Total |
|---|---|---|---|
| All events | 68,981 | 0.00 | 100 |
| Singlets 1 | 42,925 | 62.23 | 62.23 |
| Singlets 2 | 35,649 | 83.05 | 51.68 |
| CD3-FITC | 253 | 0.71 | 0.37 |
| PD-1 positive | 154 | 60.87 | 0.22 |
| PD-1 negative | 35 | 13.83 | 0.05 |

TABLE 49
C14503-1010-101/PE FMO (Data Source −1)

| Name | Events | % Parent | % Total |
|---|---|---|---|
| All events | 71,302 | 0.00 | 100 |
| Singlets 1 | 43,284 | 60.71 | 60.71 |
| Singlets 2 | 35,163 | 81.24 | 49.32 |
| CD3-FITC | 18,272 | 51.96 | 25.63 |
| PD-1 positive | 101 | 0.55 | 0.14 |
| PD-1 negative | 17,888 | 97.90 | 25.09 |

TABLE 50
C14503-1010-101/Post Digest Sort (Data Source −1)

| Name | Events | % Parent | % Total |
|---|---|---|---|
| All events | 100,000 | 0.00 | 100 |
| Singlets 1 | 60.071 | 60.07 | 60.07 |
| Singlets 2 | 48,908 | 81.42 | 48.91 |
| CD3-FITC | 18,288 | 37.39 | 18.29 |
| PD-1 positive | 1,910 | 10.44 | 1.91 |
| PD-1 negative | 13,698 | 74.90 | 13.70 |

TABLE 51
C14503-1010-101/Post Digest Sort Purity (Data Source −1)

| Name | Events | % Parent | % Total |
|---|---|---|---|
| All events | 1,022 | 0.00 | 100 |
| Singlets 1 | 610 | 59.69 | 59.69 |
| Singlets 2 | 512 | 83.93 | 50.10 |
| CD3-FITC | 313 | 61.13 | 30.63 |
| PD-1 positive | 176 | 56.23 | 17.22 |
| PD-1 negative | 24 | 7.67 | 2.35 |

### PD-selected TIL Process Day 11

### Preliminary Operations

Recorded starting time and date of CM2 incubation. Incubation of CM2 media bag overnight prior to processing. Ensured pre-warmed warm packs were available.

Prepared feeder bag and G-Rex 500MCS flask with CM2. Removed CM2 from the incubator. Recorded date and time of removal. Determined if the elapsed CM2 incubation time was acceptable. CM2 should be incubated at least overnight.

Closed all clamps on Pooled Feeder Cells. Sterile welded the CM2 bag to Pooled Feeder Cells. Placed Pooled Feeder Cells on the analytical balance and tare. Transferred by gravity approximately 500±10 mL of CM2 into Pooled Feeder Cells. Recorded the volume transferred. Note: 1 g was equivalent to 1 mL.

Heat sealed and removed Pooled Feeder Cells from the CM2 bag. Transferred Pooled Feeder Cells into the BSC.

Transferred a pump boot and a G-Rex 500MCS flask into the BSC. Ensured all clamps on the flask were closed except the large filter line. Using the luer connections, connected the outlet line of the pump boot to the red line on the flask. Sterile welded the inlet line of the pump boot to the CM2 bag. Placed the G-Rex 500MCS on a scale and tare.

Pumped CM2 into the G-Rex 500MCS flask until the volume reached 4000±10 mL. Recorded the volume added to flask. Clamped and heat sealed. Placed the flask in the incubator until needed. Recorded the incubator ID. Note: 1 g was equivalent to 1 mL

Prepared feeder cells. Retrieved three bags of feeder cells from at least two different lots. Recorded the lot numbers. Visually inspected the bags to ensure they were acceptable (free of any cracks, broken ports, and bad seals). Recorded the temperature of the water bath. Recorded the start time of the thaw for all three feeder bags. Recorded the stop time of the thaw for all three feeder bags. Pooling and counting feeder cells.

Prepared a Feeder Cell Harness by spiking, sterile welding or attaching as necessary the appropriate connections below. Ensured at a minimum there were three spikes available on one side of the harness. The harness should have a three-way stopcock at the center. Additionally, one luer/spike connection must be available on the opposite side of the harness to heat seal Pooled Feeder Cells in step 4.2. Recorded components used to make the harness. Note: Any connections that was not be used were heat sealed.

Sterile welded or attached Pooled Feeder Cells to the Harness. Feeder Cell Harness with Pooled Feeder Cells attached (1). Labeled the cryovials as Day 11 Feeder Cells 1-4. Opened a new 100 mL syringe and drew 20 mL of air. Replaced the syringe on the Feeder Cell Harness with the new 100 mL syringe. Spiked the single port of each of the three thawed feeder cell bags with a spike from the feeder cell harness. Rotated the stopcock so that Pooled Feeder Cells was in the off position. Placed warm packs under Pooled Feeder Cells.

Opened all clamps to the thawed feeder cell bags. Drew up the entire contents of all feeder cells bags pooling the contents of all bags together. Recorded the total volume of feeder cells recovered.

Rotated the stopcock so that the thawed feeder bags were in the off position. Opened all clamps in the direction of Pooled Feeder Cells. Transferred the cells from the syringe to Pooled Feeder Cells.

Attached a 10 mL syringe to the NIS port, mixed the bag well, removed a 1 mL sample, and placed the sample in a cryovial. Repeated using a fresh 10 mL syringe and cryovial to obtain a total of four 1 mL samples.

Calculated the volume in Pooled Feeder Cells. If necessary, diluted the sample in AIM-V (a 1:10 dilution was recommended to start). Recorded dilution factor. Repeated for all four samples and record data below. Note: Optimal concentration range for the NC-200 was between 5×10⁴ and 5×10⁶ cells/mL undiluted. Counts outside this range would prompt the user with a message. Calculated the average viable cell concentration and viability of the four counts as follows: (Count 1+Count 2+Count 3+Count 4)÷4 Calculated the total viable cells in Pooled Feeder Cells. Was TVC in Pooled Feeder Cells more than 5×10⁹ cells?

Thawing of additional feeder cells. Requested an additional bag of feeder cells to thaw. Recorded lot numbers of feeder cells used.

Visually inspected the bag to ensure they were acceptable (free of any cracks, broken ports, and bad seals). Recorded the temperature of the water bath. Recorded the start time of the thaw for the feeder bag. Recorded the stop time of the thaw for the feeder bag. Visually inspected the bag to ensure they were acceptable (free of any tears and leaks).

Replaced the syringe on the Feeder Cell Harness with a fresh 100 mL syringe. Spiked the single port of the thawed feeder cell bag with a spike from the Feeder Cell Harness. Added an additional spike by sterile welding, if necessary. Rotated the stopcock so that Pooled Feeder Cells was in the off position. Placed warm packs under Pooled Feeder Cells.

Opened clamp to the thawed feeder cell bag. Drew up the entire contents of the thawed feeder cell bag with a single draw. Recorded the total volume of feeder cells recovered.

Rotated the stopcock so that the thawed feeder bag was in the off position. Opened all clamps in the direction of Pooled Feeder Cells. Transferred the cells from the syringe to Pooled Feeder Cells.

Attached a 10 mL syringe to the NIS port, mixed the bag well, removed a 1 mL sample, and placed the sample in a cryovial. Repeated using a fresh 10 mL syringe and cryovial to obtain a total of four 1 mL samples.

Calculated the new volume in Pooled Feeder Cells. If necessary, diluted the sample in AIM-V (a 1:10 dilution was recommended to start). Recorded dilution factor. Repeated for all four samples and record data below. Note: Optimal concentration range for the NC-200 was between 5×10⁴ and 5×10⁶ cells/mL undiluted. Counts outside this range would prompt the user with a message.

Calculated the average viable cell concentration and viability of the four counts as follows: (Count 5+Count 6+Count 7+Count 8)/4. Calculated the total viable cells in Pooled Feeder Cells. Was TVC in Pooled Feeder Cells more than 5×10⁹ cells?

Addition of feeder cells to G-Rex 500MCS. Calculated the volume necessary for 5×10⁹ viable cells. Calculated the volume to remove from Pooled Feeder Cells to leave 5×10⁹ cells in the bag. Removed the volume calculated from Pooled Feeder Cells to leave 5×10⁹ cells in the bag. Recorded volume removed. Used a new syringe with enough air to clear the line to Pooled Feeder Cells, if necessary.

Using a 1 mL syringe with 18 G needle attached drew up 0.15 mL of OKT-3. Removed the needle and dispensed OKT-3 into Pooled Feeder Cells via the NIS port. Flushed the syringe with 0.5 mL of feeder cells to ensure all OKT-3 was added into the bag. Clear the line. Ensured there was enough air in the feeder bag to facilitate gravity flowing into the G-Rex 500MCS flask, then heat sealed off Pooled Feeder Cells, leaving enough tubing for future welding.

Removed the G-Rex500MCS from the incubator and place besides the sterile welder. Sterile welded Pooled Feeder Cells to the red line on the G-Rex 500 MCS. Hung Pooled Feeder Cells on an IV pole and allowed the entire contents to flow from the bag into the G-Rex 500MCS flask by gravity. Ensured line was clear after addition of feeder cells.

Closed the clamps and heat sealed Pooled Feeder Cells off of the flask. Labeled the flask as TIL Culture+Feeder Cells (Day 11) and placed the flask in the incubator. Recorded the time the flask was returned to the incubator and the incubator ID.

### Prepared TIL

Labeled an EV1000N bag or EXP-1L as TIL Harvest. Heat sealed one of the female luer connections and removed the clamp. Weighed the empty bag and recorded the weight. Label an appropriately sized bag as Waste. Sterile welded Waste bag to the red line on the G-Rex 100MCS flask.

If processing PD-1-selected TIL, a blood filter was not necessary and the clear collection line of the G-Rex 100MCS could be welded directly to the TIL Harvest bag. If processing Gen 2.1 TIL, sterile welded one of the inlet tubing lines of a blood filter to the clear collection line of the G-Rex 100MCS flask. Heat sealed the other inlet line to prevent leaking. Sterile welded the outlet line of the filter to the TIL Harvest bag.

Removed approximately 900 mL of supernatant from the first G-Rex 100MCS flask. When supernatant removal was complete, placed TIL Harvest on top of the Waste bag to use as a warm pack. Swirled and tapped the flask until all cells had been detached from the membrane. If the cell collection straw was not at the junction of the wall and the membrane, rapped the flask while tilted at a 45° angle to properly position the straw. Slowly tilted the flask towards the collection tubing so the fragments remained on the opposite side of the flask, if applicable.

While keeping the G-Rex 100MCS flask tilted, transferred the remaining cell suspension to the TIL Harvest bag. Held the blood filter vertically to allow it to fill with the suspension. Avoided allowing tumor fragments to transfer out of the G-Rex 100MCS flask, if applicable.

When cell collection had completed, closed the clamps on the clear line and opened the clamps to the red media line. Gently squeezed the Waste bag to start the flow of media into the flask and continue filling until a third to a half of the membrane on the bottom of the flask was covered. Clamped the red line. Swirled and tapped the flask vigorously and opened the clamps on the clear line. Collected the remaining cell suspension. Once collection was complete, clamped and heat sealed the red and clear lines.

Repeated steps 7.2 through 7.10 until all G-Rex 100MCS flasks had been harvested. Discarded the Waste bag. Tarde the balance and took the weight of the TIL Harvest bag.

Calculated the volume of cell suspension in TIL Harvest. Note: Consider 1 g was equal to 1 mL.

Attached a 10 mL syringe to the NIS port, mix the bag well, removed a 1 mL sample, and placed the sample in a cryovial. Repeated using a fresh 10 mL syringe and cryovial to obtain a total of four 1 mL samples.

Calculated the new volume in TIL Harvest. Placed TIL Harvest in an incubator. Recorded the time placed in the incubator and the incubator ID

TIL cell count. If necessary, diluted the sample in AIM-V (a 1:10 dilution was recommended to start). Recorded dilution factor. Repeated for all four samples and record data below. Note: Optimal concentration range for the NC-200 was between 5×10⁴ and 5×10⁶ cells/mL undiluted. Counts outside this range would prompt the user with a message.

Calculated the average viable cell concentration, total cell concentration, and viability of the four counts as follows: (Count 1+Count 2+Count 3+Count 4)/4. Calculated the total viable cells in TIL Harvest. Was the total viable cell count in TIL Harvest more than 55×10⁶ cells?

### QC Sampling

Calculated the volume to remove 5×106 cells for flow cytometry. Removed the volume calculated above from TIL Harvest and transferred to a container labeled D11 Flow 5×10⁶ cells with the lot number and date of sampling. Sent to QC for testing. Calculated the volume of TIL Harvest remaining after QC sampling. Calculated the viable cells remaining in TIL Harvest after QC sampling. Was the total viable cell count remaining >200×10⁶ viable cells?

Calculated the volume required to seed 200×10⁶ viable cells into the G-Rex 500MCS. Calculated the volume to remove in order to retain 200×106 viable cells in TIL Harvest.

Connected an appropriately sized syringe to TIL Harvest. Removed the volume calculated above from TIL Harvest and placed in an appropriately sized conical tube labeled Excess TIL. Recorded volume removed. Placed in an incubator until further processing. Recorded incubator ID.

### Addition of TIL to G-Rex500MCS

Removed the G-Rex500MCS TIL Culture+Feeder Cells (Day 11) from the incubator and placed it next to the sterile welder. Sterile welded TIL Harvest to the red line of the G-Rex500MCS flask. Released all clamps leading from TIL Harvest to the G-Rex 500MCS flask and gravity feed the entire contents of TIL Harvest into the flask. Cleared the line, heat sealed, and discarded TIL Harvest.

Ensured that all clamps on the G-Rex 500MCS flask were closed except the large filter line. Placed flask in the incubator and recorded the incubator information. Ensured that the media in the G-Rex 500MCS flask was level when resting on the incubator shelf.

Cryopreservation of Excess TIL

Calculated the amount of CS10 to add to Excess TIL. Note: Rounded up to a whole number for volume of CS10. Cell concentration would be approximately 100×10⁶ cells/mL. Centrifuged Excess TIL at 350 G for 10 minutes at 20° C.

Using an appropriately sized serological pipette, aspirated the supernatant from Excess TIL and discarded in the waste bottle. Gently tapped the bottom of the tube to resuspend the cells in the remaining fluid. Using a needle and appropriately sized syringe drew up the volume of CS10 calculated. Added volume of CS10 dropwise into Excess TIL. Mixed well using an appropriately sized pipette. Prepared 1 mL aliquots of Excess TIL in cryovials. Recorded the number of cryovials prepared. Prepared a blank vial to be used with the vial probe in the CRF by adding 1 mL of CS10 to a 1.8 mL cryovial labeled Blank. Freeze.

Post-cryopreservation of excess TIL. Stopped the freezer after the completion of the run and recorded storage information.

### Preparation of CM4 Media for Day 16

Total volume of CM4 to prepare. Note: 25 L of media was required for one product. Batches should be prepared in increments of 10 L.

Number of CM4 bags to prepare (one CM4 bag was 5 L): n=total volume of CM4 to prepare/5 L

Selected type of AIM-V container: AIM-V container 10 L Bag. Quantity of bags necessary: A=Total quantity of CM4 to prepare/10 L; IM-V container 1L Bottle; Quantity of bottles necessary: B=Quantity of CM4 to prepare/1L.

First: IL-2 Akron prefilled syringe 1 mL ready to use.

AIM-V container 10 L Bag.

Calculated Volume of IL-2 needed to prepare one 10 L bag of CM4 at 3000 IU/mL: (10000 mL×3000 IU/mL=30×10⁶ IU by bag)→IL-2 to transfer: 30×10⁶ UI/IL-2 Specific Activity.

Calculate Total volume needed of IL-2: Volume of IL-2 by bag×Number of AIM-V 10 L bag (A)=______mL×__=______mL (1 decimal).

Calculated Number of syringes needed: Total volume of IL-2/volume per syringe=______mL/1 mL=______syringe(s) (rounded up to whole number.

AIM-V container 1L Bottle (one bag of CM4=10×1L bottle AIM-V)

Calculated Volume of IL-2 needed to prepare one 10 L bag (10000 mL×3000 IU/mL=30×10⁶ IU by bag); IL-2 to transfer: 30×10⁶ UI/IL-2 Specific Activity=______mL of IL-2 by 10 L AIM-V bag (1 decimal).

Calculated Total volume needed of IL-2: Volume of IL-2 by bag×Number of 10 L CM4 bag (n)=______mL x______=______mL (1 decimal).

Second: IL-2 Akron powder to reconstitute with 1 mL WFI

AIM-V container 10 L Bag

Calculated the number of mg of IL-2 needed to prepare one 10 L bag of CM4 at 3000 IU/mL (10000 mL×3000 IU/mL=30×10⁶ IU by bag) 30×10⁶ UI/IL-2 specific activity−______mg of IL-2 by 10 L AIM-V bag (1 decimal); IL-2 to transfer (1 mg=1 mL)=______mL of IL-2 by 10 L AIM-V bag (1 decimal).

Calculated the total number of mg needed of IL-2: Number of mg of IL-2 by bag x number of AIM-V 10 L bag (A)=______mg x______=______mg (1 decimal).

Calculated the number of vials needed (1 vial contain 1 mg):______vial(s) (rounded up to a whole number).

AIM-V container 1L Bottle (one bag of CM4=10×1L bottle AIM-V

Calculated the number of mg of IL-2 needed to prepare one 10 L bag of CM4 at 3000 IU/mL (10000 mL×3000 IU/mL=30×10⁶ IU by bag) 30×10⁶ UI/IL-2 specific activity=______mg of IL-2 by 10 L CM4 bag (1 decimal); IL-2 to transfer (1 mg=1 mL)=______mL of IL-2 by 10 L CM4 bag (1 decimal).

Calculated the total number of mg needed of IL-2: Number of mg of IL-2 by bag×number of CM4 10 L bag (n)=______mg x______=______mg (1 decimal).

Calculated the number of vials needed (1 vial contain 1 mg): vial(s) (rounded up to a whole number).

Last: IL-2 Cellgenix Powder to Reconstitute with 2 mL Acetic Acid

AIM-V container 10 L Bag

Calculated the number of mg of IL-2 needed to prepare one 10 L bag of CM4 at 3000 IU/mL (10000 mL×3000 IU/mL=30×10⁶ IU by bag) 30×10⁶ UI/IL-2 specific activity−______mg of IL-2 by 10 L AIM-V bag (1 decimal); IL-2 to transfer (1 mg=1 mL)=______mL of IL-2 by 10 L AIM-V bag (1 decimal).

Calculated the total number of mg needed of IL-2: Number of mg of IL-2 by bag× number of AIM-V 10 L bag (A)=______mg x______=______mg (1 decimal).

Calculated the number of vials needed (1 vial contain 1 mg):______vial(s) (rounded up to a whole number).

AIM-V container 1L Bottle (one bag of CM4=10×1L bottle AIM-V)

Calculated the number of mg of IL-2 needed to prepare one 10 L bag of CM4 at 3000 IU/mL (10000 mL×3000 IU/mL=30×10⁶ IU by bag) 30×10⁶ UI/Il-2 specific activity=______mg of Il-2 by 10 L CM4 bag (1 decimal); IL-2 to transfer (1 mg=1 mL)=______mL of IL-2 by 10 L CM4 bag (1 decimal)

Calculated the total number of mg needed of IL-2: Number of mg of IL-2 by bag×number of CM4 10 L bag (n)=______mg x______=______mg (1 decimal

Calculated the number of vials needed (1 vial contain 1 mg):______vial(s) (rounded up to a whole number)

IL-2 to use see in section 1: If Akron 1L-2 in prefilled syringe, no reconstitution, went to section 3. If Akron IL-2 in powder, advanced to step 2.2 to proceed with reconstitution. If Cellgenix IL-2 in powder, advanced to step 11.7 to proceed with reconstitution.

Recorded the number of vials to reconstitute. Spiked WFI bottle, using a 10 mL syringe, drew 1 mL of WFI. Connected an 18 G needle to the syringe and transfer 1 mL WFI into IL-2 vial(s). Inverted the vial(s) 2-3 times and swirl until all powder was dissolved. Avoided foam formation and did not mix vigorously. Repeated this step to reconstitute the needed number of vials (use a new syringe if needed) Kept hl BSC until use. Advanced to section 12 fit using 10 L AIM-V Lags or section 4 if using 1L bottle of A1M-V.

Using a pumpmatic pipette or 10 mL syringe with 18 G needle, drew 2 mL of HAc per vial to reconstitute. Connected an 18 G needle, if needed, to the syringe and transfer 2 mL HAc into the vial. Inverted the vials 2-3 times and swirled until all powder was dissolved.

Avoided foam formation and do not mix vigorously. Repeated this step to reconstitute the needed number of vials in section 1. Used 1 pumpmatic pipette per 10 mL of HAc to transfer. Kept in BSC until use. Advanced to section 3 if using 10 L AIM-V bags or section 4 if using 1L bottle of AIM-V.

CM4 media preparation with AIM-V medium 10 L Bag. Connected via luer lock the first extension set to the first CM4 media 5 L bag. Repeated this step with each CM4 media 5 L bag. Removed CM4 media 5 L bags from the BSC. Identification of 5 L Labtainer bag as CM4 media.

The quantities below had to be multiplied by the number A of AIM-V 10 L bag to prepare. Transferred the following into the BSC: The quantities below had to be multiplied by the number A of AIM-V 10 L bags for preparation of the total volume. Label 10 L bags of AIM-V as CM4 pool media #.

Using an adequate volume syringe and 18 G needle, drew the needed volume of IL-2 for one 10 L bag (see section 5) and transferred into GlutaMAX 1. Marked bottle to indicate addition. Repeated this step with each bottle of GlutaMAX needed according to the number of 10 L AIM V bags that will be prepared. One bottle of Glutamax+IL-2 would be used per each 10 L AIM V bag.

Using an appropriate sized syringe and fluid dispensing connector, transferred the volume calculated in section 5 of IL-2 into GlutaMAX 1 from the prefilled syringe. Marked bottle to indicate addition. Repeated this step with each bottle of GlutaMAX needed according to the number of 10 L AIM V bags that would be prepared. One bottle of Glutamax+IL-2 would be used per each 10 L AIM V bag.

Transferred the first CM4 pool media bag into the BSC. Spiked the first CM4 pool media with a 4″ plasma transfer set and attached via luer lock an extension set. Repeated this step with each bag of CM4 pool media.

Transferred the following into the BSC: Pump boot (1). Connect the Pump boot to CM4 Pool media 1.

Placed the Pump boot into the Acacia pump and set the parameters.

In the BSC, connected the remaining end of the pump boot to a pumpmatic pipette. Using the pumpmatic pipette transferred the entire contents of one GlutaMAX bottle into one CM4 pool media bag. Marked each bag prepared when complete. i.e. GlutaMAX 1 would be transferred into CM4 pool media bag 1 and so on. Repeated this step for each bag of CM4 pool media.

Sterile welded to the CM4 media bag. Manually primed the line leading to CM4 media using speed 100 RPM. Placed the CM4 media bag on the balance and tare. Set the parameters of the Acacia pump. Pumped 4900±10 mL of media from the CM4 pool media bag to the CM4 media bag. Recorded volume. Close clamps and heat sealed to remove the filled CM4 media bag. Heat sealed off each CM4 pool media after filling two CM4 media bags. Retained for sterility testing. Repeated for remaining CM4 media bags. Identification of CM4 pool media bags as Sterility #.

Recorded the volume of CM4 added to each CM4 media bag. Bags were stored at 2-8° C.

CM4 media preparation with AIM-V medium 1L bottles. The quantities below had to be multiplied by the number of CM4 bags to prepare.

Using an adequate volume syringe and 18 G needle, drew the needed volume of IL-2 for one 10 L bag (see section 1) and transferred into bottle of GlutaMAX 1. Repeated this step with each bottle of GlutaMAX needed according to the number of CM4 pool media bags that would be prepared. One bottle of Glutamax+IL-2 would be used per each CM4 pool media bag

Using an appropriately sized syringe and fluid dispensing connector, transferred the volume calculated in section 1 of IL-2 into GlutaMAX 1 from the prefilled syringe. Repeated this step with each bottle of GlutaMAX needed according to the number of CM4 pool media bags that would be prepared. One bottle of Glutamax+IL-2 would be used per each CM pool media bags.

Transferred the first CM4 pool media bag into the BSC. Attached an extension set to CM4 pool media, if needed. Transferred a pump boot into the BSC. Connected the pump boot to CM4 Pool media.

Placed the pump boot into the Acacia pump and set the parameters. Connected the remaining end of the pump boot to a pumpmatic pipette. Using the pumpmatic pipette pumped the entire contents of one GlutaMAX+IL-2 1 into one CM4 pool media bag. Continued with 10×1L bottles of AIM-V. Repeated for all CM4 pool media bags, adding extension sets as necessary. Marked each bag prepared when complete. i.e. GlutaMAX 1 would be transferred into CM4 pool media 1 and so on. Attached an extension set to all CM4 media bags. Sterile welded the end of the pump boot with the red cap onto the CM4 media bag. Manually primed the line leading to CM4 media using speed 100 RPM.

Placed the CM4 media bag on the balance and tare. Set the parameters of the Acacia pump. Pumped 4900±10 mL of media from the CM4 pool media bag to the CM4 media bag. Recorded volume in step 4.13. Closed clamps and heat sealed to remove the filled CM4 media bag. Heat sealed off each CM4 pool media after filling two CM4 media bags. Retained for sterility testing. Repeated steps 4.8-4.10 for remaining CM4 media bags.

Identification of CM4 pool media bags as Sterility #. Mark sterility option: If testing sterility in house: Remove 20 mL of media from Sterility #bag. Inoculate 10 mL into an anaerobic BacT/Alert bottle and 10 mL into an aerobic BacT/Alert bottle. Bag could be discarded after sampling. If sending out for testing: Placed the Sterility #bag at 2-8° C. Sterility would be done by sending sample to an outside vendor for sterility by membrane filtration.

Recorded the volume of CM4 added to each CM4 media bag. Stored bags at 2-8° C.

### PD-1 Selected TIL Process Day 16

Recorded starting time and date of CM4 incubation. Incubation of CM4 media bag overnight prior to processing. Recorded start time of manufacturing.

Closed all clamps on the 10 L Labtainer bag for supernatant collection. Labeled the bag Supernatant. Using a luer, attached an extension set. Removed the G-Rex 500MCS flask from the incubator and placed next to the GatheRex. Checked that all clamps were closed except large filter line.

Sterile welded the Supernatant bag to the red harvest line on the G-Rex. Labeled an EV1000N bag or EXP-1L as Cell Fraction (CF). Heat sealed one line of the bag close to the end and removed the clamp. Recorded the dry weight of the CF bag.

Identification of Cell Fraction (CF) bag. Sterile welded the CF bag to the clear line of the G-Rex 500MCS. Inserted red and clear lines in the corresponding slots of the GatheRex. Connected the GatheRex line to the filter line on the G-Rex 500MCS. Supernatant Collection: Released all clamps leading to the Supernatant bag and reduced flask volume.

StartGatheRex. GatheRex would stop when air entered the line. When completed, closed the clamp.

### Cell Fraction Collection

Placed CF bag on top of the Supernatant bag during collection to keep warm. Alternatively placed on warm packs that had been conditioned in the incubator overnight. Recorded TIL harvest initiation time.

Tapped the flask and swirled media to release cells from the membrane, checked if all cells have detached. Ensured the hose was at the edge of the flask and in the fluid by tilting it. Maintained the edge tilted during the next step.

Released the clamps leading to the CF bag. Started the GatheRex to collect the cell fraction. When done, closed the clamps.

Closed the clamps on the cell collection line and opened the clamps on the red media line. Back washed and transferred more cells to the CF bag by the following steps. Released clamps on the red line. Allowed enough media to flow into the flask by gravity to cover ⅓ of the bottom of the G-Rex 500MCS surface. Closed the clamp. If cells were still adhered to the membrane, repeated back wash steps and be careful to not over inflate the cell suspension bag with air from repeated back flushes.

Closed all clamps and heat sealed near the previous seal on the red line and clear line. For the CF bag left the same length of tubing as when dry weight was recorded in step 2.6. Discarded the G-Rex 500MCS, it would not be reused in the culture split. Retained Supernatant bag for further use.

Calculated the volume of the CF. Considered 1 g was equal to 1 mL.

Attached a 10 mL syringe to the NIS port, mixed the bag well, removed a 1 mL. Wiped the NIS port of CF with an alcohol wipe. Attached a 10 mL syringe to the NIS port, mixed the bag well, removed a 1 mL.

Transferred CF bag in incubator until needed. Calculated the volume of remaining Cell Fraction (CF).

Performed cell count using Viability and Cell Count Iovance on the NC-200. If necessary, diluted the sample in AIM-V (a 1:10 dilution was recommended to start). Recorded dilution factor. Repeated for all four samples and record data below. Note: Optimal concentration range for the NC-200 was between 5×10⁴ and 5×10⁶ cells/mL undiluted. Counts outside this range would prompt the user with a message.

Calculated the average viable cell concentration and viability of the four counts as follows: (Count 1+Count 2+Count 3+Count 4)÷4. Calculated the volume of CF to remove 1×10⁶ cells for mycoplasma sample.

Labeled three 15 mL conical tubes Mycoplasma D16 1-3 and two 15 mL conical tubes Mycoplasma D16 Reference 1-2. Included sampling date on label.

Using an appropriately sized syringe removed the volume of Cell Fraction determined above, (step 3.16) and transferred into the tube labelled Mycoplasma D16 1. Repeated sampling with a new syringe for Mycoplasma D16 2, Mycoplasma 016 3, Mycoplasma Reference D16 1, and Mycoplasma Reference D16 2. Placed the CF bag back in the incubator. Calculated total volume removed.

Calculated the volume of supernatant necessary for each Mycoplasma D16 tube to have 10 mL total.

Using an appropriately sized syringe, removed the volume of supernatant from the Supernatant bag calculated in step 3.20 and transferred to Mycoplasma D16 1. Repeated for all remaining tubes prepared in step 3.19.

### BacT/Alert Sample Preparation

Using an appropriately sized syringe, flushed the syringe 3 times with 10 mL from the Supernatant bag. Removed 10 mL of supernatant. Attached an 18 G needle to the syringe and injected 5 mL in one anaerobic bottle and 5 mL in one aerobic bottle.

Calculated number of G-Rex500MCS to seed. Calculated the volume of Cell Fraction remaining. Calculated total viable cells remaining. Calculated the number of G-Rex 500MCS to seed. Rounded up to the nearest whole number. If amount of G-Rex 500MCS to seed >5, the number of G-Rex 500MCS to seed would be 5. Cell suspension over 5×10⁹ would be divided evenly between all flasks. Calculated the volume of Cell Fraction to seed for each G-Rex 500MCS flask. Seeding G-Rex500MCS flask(s) with TIL. G-Rex flasks required were to be opened in the BSC. Ensured luer locks were secure and any plastic clamps were closed besides the large filter line. Labeled new G-Rex flasks.

Sterile welded Cell Fraction bag to the red line on the G-Rex 500MCS flask. Hung the bag and ensured regular mixing of the Cell Fraction bag. Placed the G-Rex flask on the analytical balance and tare. The line must be cleared on the first G-Rex before taring the next; otherwise the weight was not correct.

Unclamped all lines and transferred by gravity the calculated volume (step 5.5) of Cell Fraction by weight into the G-Rex 500MCS flask 1. Considering 1 g equal to 1 mL transferred to the G-Rex 500MCS flask. Recorded the amount of cell fraction added to flask. Once the required volume was transferred by gravity to the G-Rex flask, closed the clamps near tubing closer to the G-Rex to stop addition of TIL into the flask. Cleared the line and heat sealed the red tubing and kept enough tubing for next weld as needed. Placed G-Rex in incubator.

G-Rex 2, 3, 4, 5: For additional new G-Rex 500MCS flask, repeated the same operation as for seeding. Recorded the cell suspension added volume. Recorded ending time of TIL addition

### Media Addition

Removed CM4 media bag(s) from incubator. Sterile welded one end of the pump boot to the CM4 media bag. Sterile welded the other end of the pump boot to the red line of a/the G-Rex 500MCS, Hung CM4.

Set up the pump tubing and program the pump with the following settings: Program: Volume; Speed 300 rmp. Made a mark on the graduations of the G-Rex 500MCS flask at the 5000 mL mark. Pumped CM4 into the flask up to the mark on the graduations. Heat sealed and removed the flask. Placed in an incubator at 37° C. and 5% CO2. Repeated for the remaining flasks. Recorded time of incubation start (time last flask was placed in incubator), temperature and CO2 reading of the incubator.

PD-1 selected TIL Process Day 22

Wash buffer preparation (1% HAS/PlasmaLyte A). Closed all clamps on a 51 labtainer and identify as Plasmalyte 1% HSA Wash Buffer. Prepared wash buffer expires in one day.

Using the luer connections, attached the extension set to the 5 L Labtainer bag. Spiked each HSA bottle with a mini spike and using appropriately sized syringes, transferred 125 mL of 25% HSA to the 5 L Labtainer. Recorded volume added. Sterile welded a pump boot to the extension set attached to the 5 L Labtainer. Closed all clamps on a 4S-4M60 or equivalent harness. Spiked each of the 1L bags of PlasmaLyte. Removed the PlasmaLyte bags from the BSC and sterile welded a connection on the opposite side of the harness from the PlasmaLyte bags to the remaining end of the pump boot. Opened all the clamps leading to the PlasmaLyte bags and pumped the entire volume into the 5 L Labtainer. Recorded volume added. Heat sealed the line and remove 5 L Labtainer. Left enough line for future welding.

Placed Plasmalyte 1% HSA Wash Buffer back inside the BSC. Using a 50 mL syringe, transferred 50 mL of wash buffer to a CS750 bag. Labeled the cryobag as Blank Containing LOVO Wash Buffer with the manufacturing lot number, initials, and date. The wash buffer may be kept outside the BSC at room temperature until needed again.

Selected if IL-2 would be prepared fresh or was prepared in advance.

Using an appropriately sized syringe, transferred 40 mL of wash buffer into the 50 mL conical tube labeled IL-2 6×10⁴ IU/mL. Retained this tube in the BSC for the IL-2 preparation.

### IL-2 Preparation (Proleukin)

Recorded the following information from the label on the 1L-2 vial. Reconstituted IL-2 per the manufacturer's instructions. Stored reconstituted IL-2 at 2-8° C. until ready for use.

Calculated the volume of reconstituted IL-2 to add to the wash buffer. Note: 6.0×10⁴ IU/mL×40 mL=2.4×10⁶ IU

Using a syringe and needle, removed the volume of reconstituted IL-2 calculated above and transfer to the 50 mL conical tube labeled IL-2 6×10⁴ IU/mL.

### Pre-Harvest Preparation

Recorded the number of G-Rex 500MCS flasks that would be processed. Calculated the number of 10 L Labtainers required to collect the supernatant from all of the flasks, rounding up to the nearest whole number.

Determined how many EV3000N, EXP-3L, or equivalent bags would be required to harvest the cell suspension. Two or fewer flasks would require one bag. Three to four flasks would require 2 bags. Five flasks would require 3 bags. Did not harvest more than 2 flasks into a single bag. Note: Did not overfill EV3000N or EXP-3L bags; the max fill volume was 2000 mL. Closed the clamps on the 10 L Labtainers and attached an extension set to each Labtainer by the luer connections and labeled the bags Supernatant with a printed label or marker. If two or more 10 L waste Labtainers would be used, a second GatheRex pump may be used concurrently. While the first G-Rex 500MCS was being volume reduced, the next flask could be prepared for volume reduction.

G-Rex 500MCS harvest and cell concentration with LOVO. Recorded start time of cell harvest from the first G-Rex 500MCS flask.

Removed the supernatant from the G-Rex 500MCS flask using the GatheRex Swirled the G-Rex 500MCS flask to detach cells from the membrane. Released the clamps leading to the Cell Collection Pool. Started the GatheRex to begin collecting cells via the clear line. Gently agitated the flask while cell collection was in progress to keep cells in suspension. Maintain the flask tilted so the collection straw was positioned in the corner of the flask, where it could collect all of the cell suspension. When cell collection had completed, closed the clamps on the clear line.

Proceeded to a back wash: Released clamps on the red line. Allowed enough media to flow into the flask by gravity to cover ⅓ of the bottom of the G-Rex 500MCS. Closed the clamp on the red line and vigorously tapped and swirled the flask to release cells. Transferred the cell fraction to the Cell Collection Pool. If cells were still adhered to the membrane, repeated the back wash steps. Be careful to not over inflate the cell suspension bag with air or product.

Repeated until all G-Rex 500MCS flasks had been harvested (volume reduced and cells collected).

Labeled five 15 mL conical tubes as follows:
Supernatant—Mycoplasma 1
Supernatant—Mycoplasma 2
Supernatant—Mycoplasma 3
Supernatant—Mycoplasma Reference 1
Supernatant—Mycoplasma Reference 2

Using a 50 mL syringe, removed 50 mL of supernatant form the Supernatant bag(s) according to the below sampling plan, to obtain aa sample.

Aliquoted 10 mL of supernatant into each 15 mL conical tube. Stored at 2-8° C. until transferred to QC. Discard the Supernatant bag(s). Upon completion of transfer, closed all clamps and heat seal the LOVO Source bag, using the mark on the other tubing port as a guide, to ensure the tubing length was approximately equal to when the dry weight was taken.

Weighed the LOVO Source bag containing the cell suspension. Calculated the cell fraction volume (CF). Considered 1 g equal to 1 mL.

Mixed the LOVO Source bag well. Used a 10 mL syringe to remove 1 mL of the cell fraction via the NIS, transferring to a 1.8 mL cryovial. Repeated for the next 3 cryovials, using a new 10 mL syringe for each aliquot. Labeled the cryovials 1-4. Place the LOVO Source bag in the incubator. Recorded the incubator and time.

Re-calculated the volume in the LOVC, Source bag after having removed four 1 mL samples.

If necessary, diluted the sample in AIM-V (a 1:10 dilution was recommended to start). Recorded dilution factor. Repeated for all four samples and recorded data below. Note: Optimal concentration range for the NC-200 was between 5×10⁴ and 5×10⁶ cells/mL undiluted. Counts outside this range would prompt the user with a message.

Calculated the average of the four counts (Count 1+Count 2+Count 3+Count 4)/4; Average Total Viable Cell concentration. Average Total Cells concentration. Average % Viability

Calculated the number of total cells (pre-LOVO): Average total cell concentration x Volume of LOVO Source Bag.

Calculated the total viable cells (pre-LOVO): Average total viable cell concentration×Volume of LOVO Source Bag.

In some embodiments, if total cells were >5×10⁹, remove 5×10⁸ cells to be cryopreserved as MDA retention samples. 5×10⁸/Total cell concentration=Volume to remove. If total cells were s 5×10⁹, remove 4×10⁶ cells to be cryopreserved cryopreserved as MDA retention samples. 4×10⁶/Total cell concentration=Volume to remove. Use an appropriately sized syringe to remove the required volume from LOVO Source Bag and place in a 50 mL conical tube labeled MDA Retention, Retained in incubator until cryopreservation steps.

Calculated the volume remaining in LOVO Source Bag. Volume of LOVO Source Bag−Volume removed for MDA retention vials=Volume remaining in LOVO Source Bag.

Determined if the total cells remaining in LOVO Source Bag were more than 150×10⁹. Volume remaining in LOVO Source Bag×Total cell concentration=Total cells remaining in LOVO Source Bag.

Calculated the number of cells to remove to retain 150×10⁹ viable cells. Total cells remaining in LOVO Source Bag—150×10⁹ cells=Number of cells to remove from LOVO Source Bag

Calculated the volume of cells to remove from LOVO Source Bag. Number of cells to remove from LOVO Source Bag/Total cell concentration (step 5.23)=Volume of cells to remove from LOVO Source Bag.

Using an appropriately sized syringe, removed and discarded the calculated volume of cell suspension.

Calculated the remaining volume of the cell fraction contained in LOVO Source Bag. Original volume of LOVO Source Bag—Volume removed to retain 150×10⁹ cells=volume remaining in LOVO source bag.

Placed LOVO Source Bag back in the incubator. Recorded the incubator ID and time. Labeled the 10 L Labtainer LOVO Waste, closed the clamps, and attached the extension set via the luer connections. Switched on the LOVO device and follow instructions for processing. End of LOVO run.

### IL-2 Addition

Connected an 18 G needle to a 3 mL syringe. Drew up the volume of IL-2 marked above from the IL-2 6×10⁴ IU/mL selected. Removed the needle from the syringe and transfer the syringe to FCF Post LOVO via the NIS on the bag. Flushed the syringe three times to ensure all IL-2 was added to product. Cleared the line with air. Recorded volume of IL-2 added.

Once the IL-2 had been added to FCF Post LOVO, attached FCF Post LOVO to the harness by welding to one of the remaining connectors (see diagram in step 7.7). Retained the clamp of the manifold.

### Final Formulation

Labeled a 50 mL conical tube FCF Retain. Attached a 100 mL syringe to the manifold/stopcock drew up the volume of CS10 to add to FCF Post LOVO. Dispensed the CS10 into the FCF Post LOVO bag. Retained remaining CS10 for MDA retention vials, if applicable. Recorded the time in which the CS10 finished dispensing. Recorded volume of CS10 added to FCF Post LOVO.

Verify the FCF volume to be added per DP bag, as well as the retain volume to be removed per DP bag. Manipulated a single bag at a time. Removed the syringe on the manifold and replaced it with a fresh 100 mL syringe. Opened all clamps in the direction of FCF Post LOVO, mixed the cell product well, and drew the appropriate volume of cell suspension into the syringe. Closed the clamps leading to FCF Post LOVO and opened them in the direction of DP Bag 1. Dispensed the contents of the syringe into DP Bag 1. Mixed DP Bag 1 well and drew up the appropriate amount of cell suspension to retain and removed all excess air from the bag. Dispensed the volume in the syringe into the 50 mL conical tube labeled FCF Retain. Recorded volume removed. Triple heat sealed the fine close to DP Bag 1, cut the middle seal, and removed DP Bag 1 from the BSC. Repeated for the remaining DP Bags.

Labeled a 50 mL conical tube as FCF Post LOVO. Drew up all remaining volume of FCF Post LOVO with a 10 mL syringe and transferred into tube labelled FCF Post LOVO. This would be used to create the satellite vials.

Start of freezing run. Placed all cryobags and applicable cryovials inside the freezer, closed the door to the CRF and recorded the freezer conditions. Waited for the sample temperature to reach 8±1° C. and waited for the chamber temperature to reach 4±1° C., then pressed Run to begin the program. Recorded start time. Calculated the time elapsed for cells in CS10.

Final Cell Formulation Count and Viability

Prepared dilutions for the FCF samples to be counted. A 1:100 dilution was recommended. (Prepare with two 1:10 serial dilutions). Optimal range for NC200 was between 5×10⁴ and 5×10⁶ cells/mL.

Used the Viability and Cell Count Iovance protocol on the NC-200. Performed cell counting on all four TIL samples. Ensured sample and diluent volumes were recorded with each sample run.

Calculated the average of the four counts: (Count+Count 2+Count 3+Count 4)/4; Average Total Viable Cell concentration (live); Average Total Cell concentration (live+dead); Average % Viability.

Calculated the number of viable cells. Viable cell concentration×Volume of FCF (number of bags×volume of bags).

Calculated the number of total cells. Total cell concentration×Volume of FCF (number of bags×volume of bag)

### Completion of CRF Run

Verified that the nucleation was present and the temperature did not go over 0° C. Once freeze run was complete, transferred vials and bags immediately to an LN2 storage tank.

### Measurement of Ifn-γ Secretion by Til Population During Current Process Gen 3

IFN-γ secretion by TILs during current Process Gen 3 at Day 22.

The following table shows the measurement results of IFN-γ secretion by TIL populations at various days during current Process Gen 3. Table 52, below.

| Lot Number | Process Day | Result |
|---|---|---|
| W329020207363 | D22 | 5428.25 pg/ml |

### Example 7: Selection and Expansion of PD-1+ TIL for Full Scale Manufacturing

### INTRODUCTION

Adoptive T cell therapy with autologous tumor infiltrating lymphocytes (TIL) has demonstrated durable response rates in a cohort of patients with metastatic melanoma (Rosenberg, S. A., et al., Clin Cancer Res, 2011. 17 (13): p. 4550-7). TIL products used for treatment are comprised of heterogenous T cells, which recognize tumor-specific antigens, mutation-derived patient-specific neoantigens, and non-tumor related antigens (Kvistborg, P., et al., Oncoimmunology, 2012. 1 (4): p. 409-418; Simoni, Y., et al., Nature, 2018. 557 (7706): p. 575-579). Studies have demonstrated that neoantigen-specific T cells contribute significantly to the anti-tumor activity of TIL (Schumacher, T. N. and R. D. Schreiber, Science, 2015. 348 (6230): p. 69-74). Strategies enriching TIL for tumor-reactivity are expected to yield more potent therapeutic products, especially in epithelial cancers known to contain a high proportion of bystander T cells (Turcotte, S., et al., J Immunol, 2013. 191 (5): p. 2217-25). Several studies have demonstrated that expression of PD-1 on TIL, a marker often associated with T cell exhaustion, identifies the autologous tumor-reactive T cells (Inozume, T., et al., J Immunother, 2010. 33 (9): p. 956-64; Gros, A., et al., J Clin Invest, 2014. 124 (5): p. 2246-59; Thommen, D. S., et al., Nat Med, 2018). This example provides a protocol designed to select PD-1 positive (PD-1+) cells to enrich the TIL product for autologous tumor-reactive T cells.

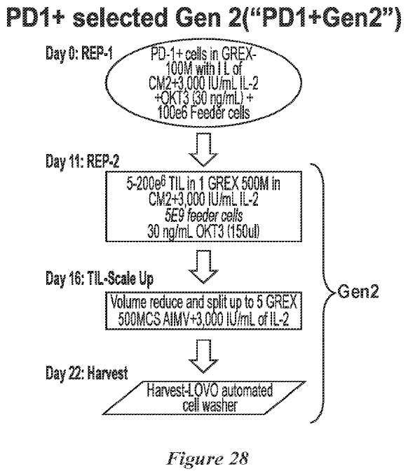

This example provides a clinical scale manufacturing protocol to sort and expand PD-1+ TIL. See, for Example FIG. 28.

This example details work regarding expanding sorted PD-1+ TIL from melanoma, lung, and head and neck cancer using a 2-REP protocol (e.g., a GEN3 based protocol as described herein). The expanded TIL are assessed for cell growth, viability, phenotype, Telomere length and function (IFNγ and Granzyme B secretion, CD107a mobilization).

This protocol will include two phases of experimentation as follows:

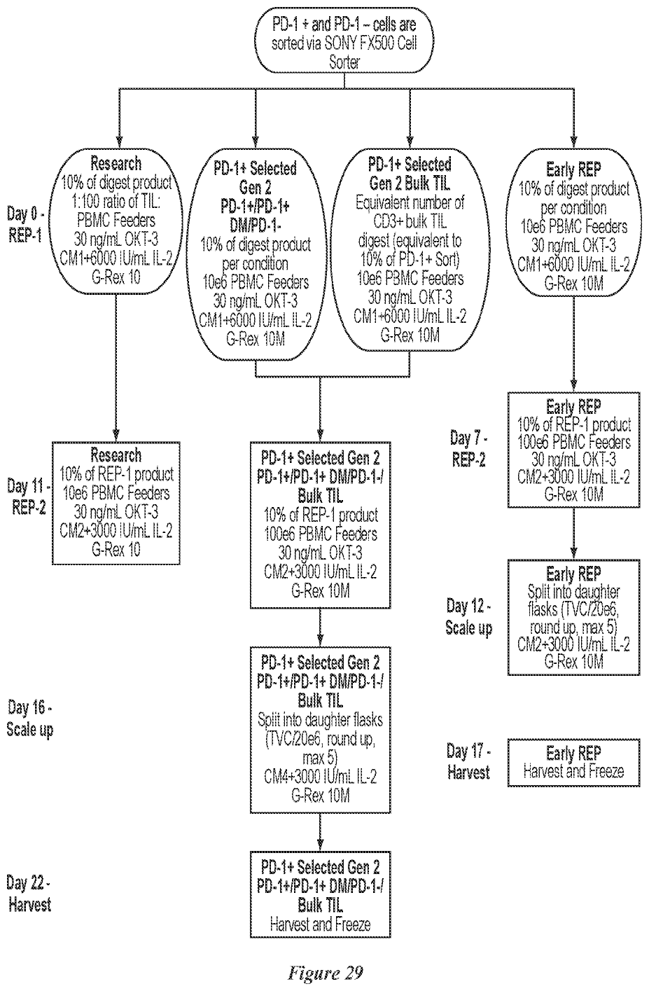

Phase 1: Feasibility study to scale up and optimize the TIL expansion process to clinical scale (see, FIG. 29).

Phase 1 will be performed to ensure that PD-1+ TIL expand adequately in the PD1+selected Gen 2 process “PD1+Gen2” (FIG. 1). Small scale cultures will be performed on PD-1+selected TIL, PD-1—selected TIL, and Bulk CD3+ TIL.

Additionally, the research protocol, defined media (CTS OpTimizer+3% SR), and a 17-day early REP process (with shortened timepoints for REP 2 initiation and split) will be tested. A brief explanation of the associated timepoints is outlined below in the methods section (FIG. 28).

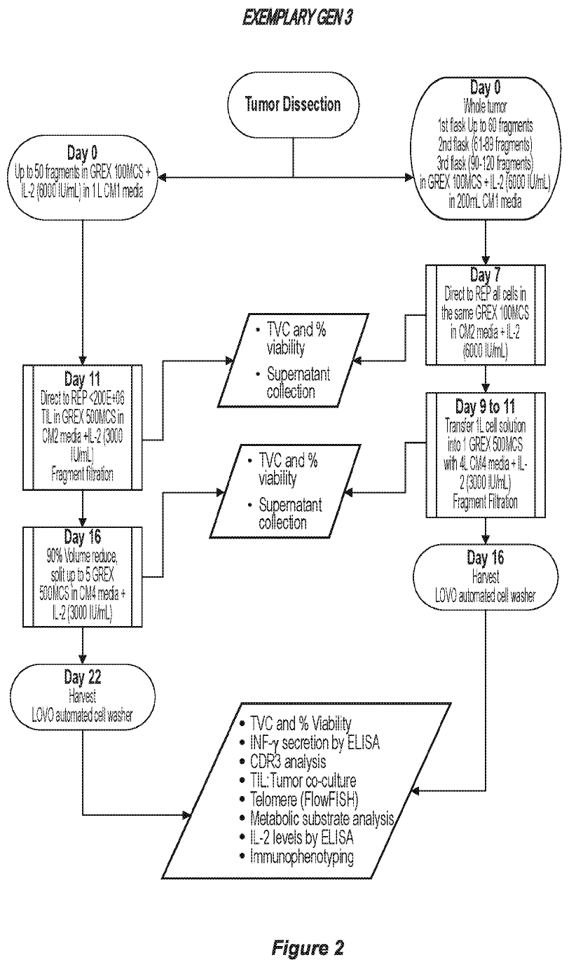

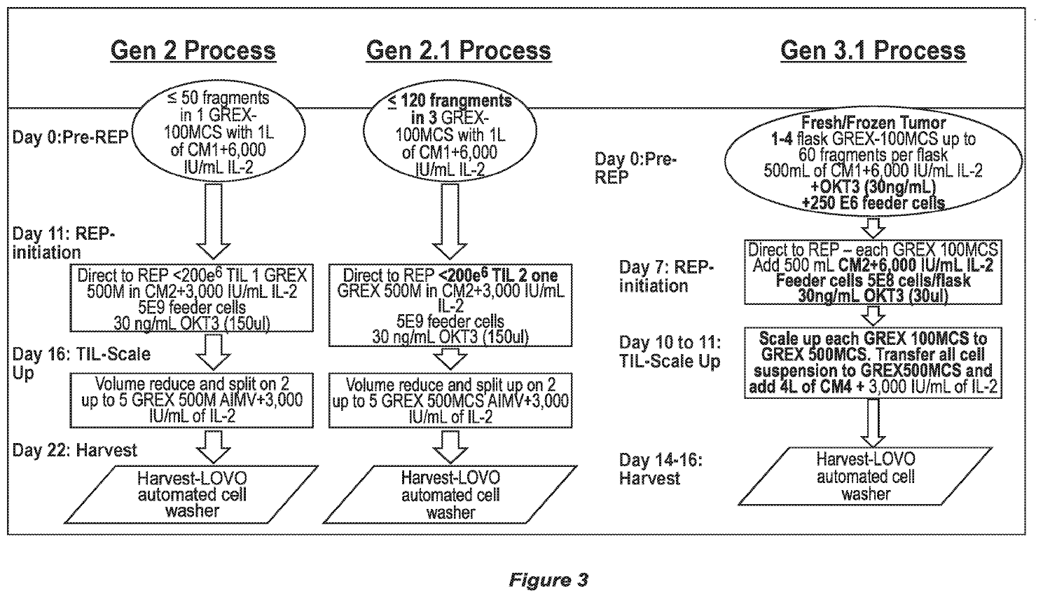

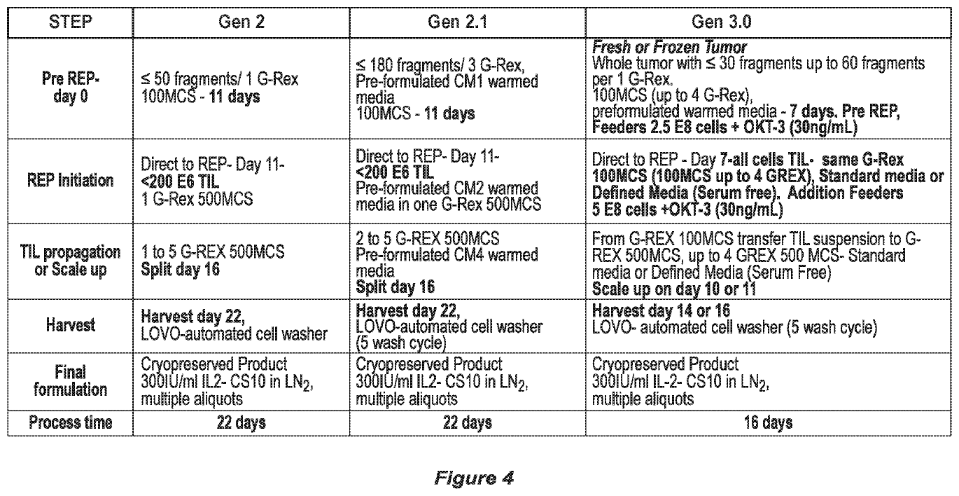

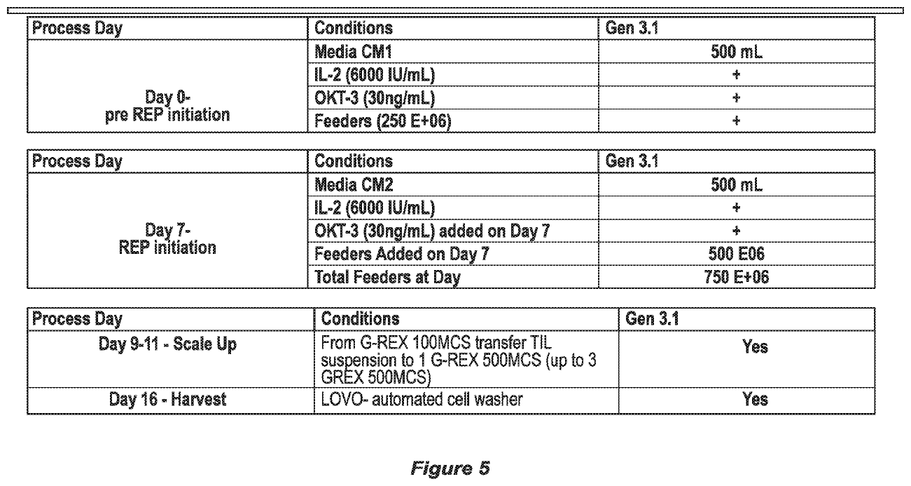

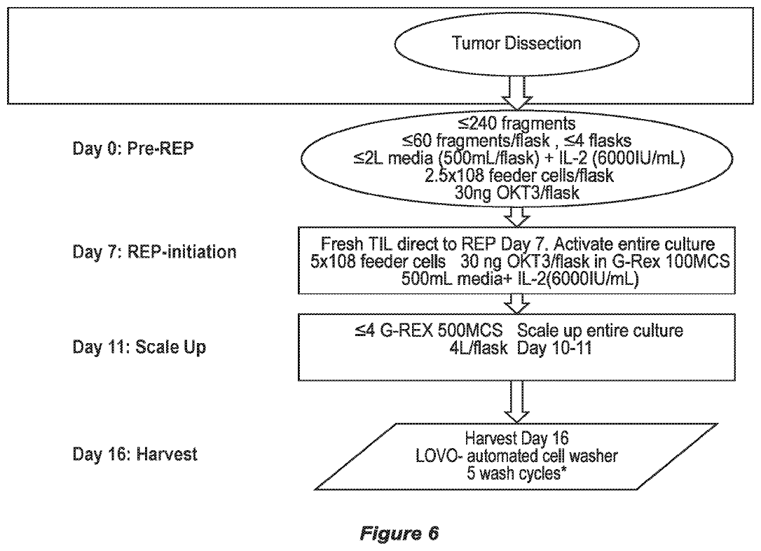

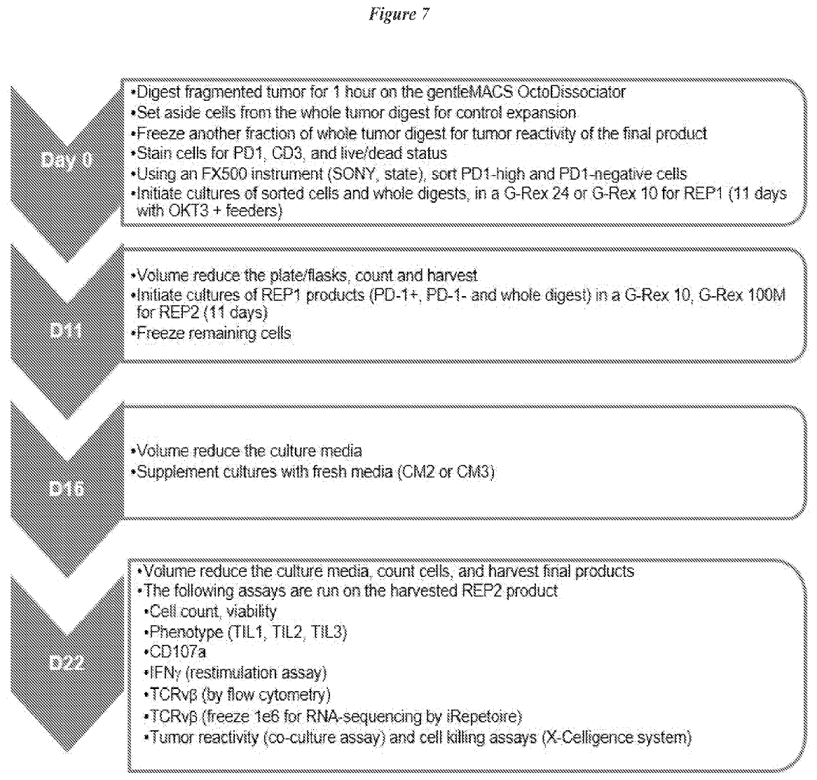

Phase 2: Test the PD1+selected Gen 2 process (Selection and Expansion) in the full scale for clinical manufacturing (FIG. 2)

Three full scale PD1+selected Gen 2 process cultures will be performed on PD-1+selected TIL per manufacturing Batch record except Day 0. For Day 0, only tumor processing and fragmentation steps will be followed per BR. Day 11 REP, Day 16 Scale up, and Day 22 Harvest will be performed per IOVA Manufacturing Batch Records as described in attachments 2-4.

On Day 0, Tumor digest will be isolated using a GMP digest cocktail containing neutral protease, DNAse I, and collagenase. The digest will be washed, stained, and flow sorted to purify PD-1+ TIL.

REP 1 will be initiated on Day 0 using purified PD-1+ TIL with 100e6 allogeneic feeder cells and 30 ng/mL OKT3 for 11 days.

REP 2 will be initiated on Day 11 using harvested REP 1 product. REP 2 (Day 11) and the subsequent Day 16 and Day 22 processes will be performed per IOVA Manufacturing Batch Records as described in attachments 2-4. A brief explanation of the associated timepoints is outlined below in the methods section (FIG. 30).

TABLE 53
Key Terms and Abbreviations

| | |
|---|---|
| ACT | ADOPTIVE CELL TRANSFER |
| BSC | BIOLOGICAL SAFETY CABINET |
| CD | CLUSTER OF DIFFERENTIATION |
| EDTA | 2,2′,2″,2′″-(ETHANE-1,2-DIYLDINITRILO)TETRAACETIC ACID |
| FACS | FLUORESCENCE ACTIVATED CELL SORTER |
| FBS | FETAL BOVINE SERUM |
| FMO | FLUORESCENCE MINUS ONE |
| FITC | FLUORESCEIN ISOTHIOCYANATE |
| GEN2 | GENERATION 2 (IOVANCE TIL EXPANSION PROTOCOL) |
| PD1+ GEN 2 | PD1+ SELECTED GEN 2 |
| IL-2 | INTERLEUKIN-2 |
| μL | MICROLITER |
| MIN | MINUTES |
| OKT3 | ANTI-CD3 ANTIBODY |
| PD-1 | PROGRAMMED DEATH RECEPTOR 1 |
| PE | PHYCOERYTHRIN |
| REP | RAPID EXPANSION PROTOCOL |
| ETOH | ETHANOL |
| SR | CTS IMMUNE CELL SERUM REPLACEMENT |

### Materials

### Tumor Tissue

Tumors of various histologies are received.

Standard Reagents for TIL Growth which Includes:

G-Rex 5M, 10M, 100M, and 500 MCS flasks (Wilson Wolf, Cat #80055S, 80110S, 81100, 85500S-CS, respectively)

GMP recombinant IL-2 (Cell-Genix, Germany, Cat #1020-1000)

Media reagents for CM1, CM2, and CM4.

Defined media reagents for CTS OpTimizer+3% SR

CTS OpTimizer SFM (Thermofisher, Cat #A1048501)

GlutaMAX 100X (Thermofisher, Cat #35050061)

Gentamycin 50 mg/mL (Thermofisher, Cat #15750060)

CTS™ Immune Cell SR (Thermofisher, Cat #A2596101)

### Flow Cytometry Staining and Analysis Reagents

Flow cytometry antibodies:
Anti-PD-1 PE, Clone EH12.2H7, Biolegend, Cat #329906
Anti-CD3 FITC, Clone OKT3, Biolegend, Cat #317306
Anti-IgG4 Fc-PE, Clone HP6025, Southern Biotech, Cat #9200-09

Sorting Buffer:HBSS with 2% FBS, sterile filtered.

Collection Buffer: hAB Serum.

### PROCEDURE

### Tumor Preparation

Processing of tumor.

Freshly resected tumor samples will be received from research alliances and tissue procurement vendors. The tumors are shipped overnight in HypoThermosol (Biolife Solutions, Washington, Cat #101104) (with antibiotic).

Remove tumor from packaging and wash 3× for 2 minutes per wash in Tumor Wash Buffer (Filtered HBSS with 50 ug/mL Gentamycin).

Fragment the entire tumor into 4-6-mm fragments in preparation for tumor digest. Keep 6 mm fragments in a well of a 6 well plate containing 10 mL of Tumor Wash Buffer.

### Enzyme Preparation for Tumor Digestion

For the Phase-1 study, tumor will be digested using Research Grade DNAse, Collagenase and Hyaluronidase prepared.

For the Phase-2 study, tumor will be digested using GMP Collagenase and Neutral Protease as described below.

Reconstitute the lyophilized enzymes in the amount of sterile HBSS indicated for each of the digestion enzymes below. Be sure to capture any residual powder from the sides of the bottles and from the protective foil on the bottles opening. Pipette up and down several times and swirl to ensure complete reconstitution.

Reconstitute the Collagenase AF-1 (Nordmark, Sweden, N0003554) in 10-ml of sterile HBSS. The lyophilized stock enzyme is at a concentration of 2892 PZ U/vial. Therefore, after reconstitution the collagenase stock is 289.2 PZ U/ml. **Note, the stock of enzymes can change so verify the concentration of the lyophilized stock and amend the final amount of enzyme added to the digest cocktail accordingly**. Aliquot into 100 uL aliquots and store at −20C

Reconstitute the Neutral protease (Nordmark, Sweden, N0003553) in 1-ml of sterile HBSS. The lyophilized stock enzyme is at a concentration of 175 DMC U/vial. Therefore, after reconstitution the neutral protease stock is 175 DMC/ml. **Note, the stock of enzymes can change so verify the concentration of the lyophilized stock and amend the final amount of enzyme added to the digest cocktail accordingly**. Aliquot into 20 uL aliquots and store at −20C

Reconstitute the DNAse I (Roche, Switzerland, 03724751) in 1-ml of sterile HBSS. The lyophilized stock enzyme is at a concentration of 4KU/vial. Therefore, after reconstitution the DNAse stock is 4KU/vial. **Note, the stock of enzymes can change so verify the concentration of the lyophilized stock and amend the final amount of enzyme added to the digest cocktail accordingly**. Aliquot into 250 uL aliquots and store at −20C.

Thaw 3 components of GMP digest cocktail and prepare the working GMP digest cocktail as follows: Add 10.2-41 of the neutral protease (0.36 DMC U/ml), 21.3-μl of collagenase AF-1 (1.2 PZ/ml) and 250-μl of DNAse I (200 U/ml) to 4.7-ml of sterile HBSS. Place the digest cocktail directly into the C-tube.

### Tumor Processing and Digestion

To the GentleMACS OctoDissociator, transfer up to (4) 6 mm tumor fragments to each GentleMACS C-Tube (C-tube) in the 5-ml of digest cocktail indicated above. Use additional GentleMACS C-Tube for additional tumor fragments.

Transfer each C-tube to the GentleMACS OctoDissociator. Digest by setting the dissociator to the appropriate program for the respective tumor histology listed below in Table 54. The dissociation will be approximately one hour.

TABLE 54
Miltenyi OctoDissociator Programs Based on Tumor Tissue Type.

| Tumor Tissue Type | Designation | Program |
|---|---|---|
| Melanoma, Ovarian, Colon, | Soft | 37C_h_TDK_1 |
| Hypopharyngeal, and Renal | | |
| Lung and Prostate | Medium | 37C_h_TDK_2 |
| Breast, Pancreatic, Hepatocellular, | Tough | 37C_h_TDK_3 |
| Head and Neck Squamous Cell | | |
| (HNSCC) | | |

Table 53: Post-digest, remove the C-tube(s) from the Octodissociator or rotator and place into the BSC. Remove the digest from each C-tube with a 25 mL serological pipette and pass the bulk digest through a 70 μm cell strainer into a 50 mL conical. Undigested parts of the tumor may not pass through the strainer, do not allow the digest to splash up due to pressure from the pipettor. Wash the C-tube(s) with an additional 10 mL of HBSS and pass the wash through the cell strainer. QS the 50 mL conical to 50 mL with HBSS.

Centrifuge the digest at 400× G for 5 minutes at RT (full acceleration & full brake).

Transfer Conical to BSC and aspirate or decant supernatant. Resuspend pellet in 5 mL of warm CM-1+6000 IU/mL IL-2 and pipette up and down 5-6 times. Perform 2 cell counts on NC-200 at no dilution per LAB-056

Place 1 mL of digest aside for CD3+Bulk control and cryopreserve 2×500 uL aliquots of digest for tumor reactivity assays. Keep digest on ice.

### Staining Digested Tumor for Flow Cytometry Analysis and Cell Sorting

Set aside a small sample (˜1e5 cells) for the PE FMO into a 15 mL conical.

The remaining tumor digest is stained with a cocktail that includes staining PD-1-PE, anti-IgG4 Fc-PE (secondary antibody for Nivolumab and Pembrolizumab) and CD3-FITC according to the following protocol. The FMO Sample will be stained with CD3-FITC only

After cell counting, add 10 mL of HBSS to digest and centrifuge at 400× G for 5 minutes at RT (full acceleration & full brake).

Transfer conical to BSC and decant supernatant. Use a micropipettor to obtain the volume of digest remaining after decanting. Add 3× this volume of Sorting Buffer to the tube. If the obtained volume is 150 uL, add 450 uL Sorting buffer, for a total volume of 600 uL.

Add 3-μl of anti-CD3-FITC per 100 μL (i.e. if volume is 600 uL, add 6×3=18 uL of antibody). (Add to both Samples.)

Add 2.5-μl anti-PD-1-PE per 100 μL (i.e. If volume is 600 uL, add 6×2.5=12.5 uL of antibody). (Do not add to FMO.)

Add anti-IgG4-Fc-PE in a 1:500 dilution (i.e. For every 500 uL of volume, add 1 uL of antibody). (Do not add to FMO.)

Mix digest gently with a 1 mL micropipettor and Incubate cells on ice for 30 minutes. Protect from light during incubation. Agitate by flicking gently every 10 minutes during incubation to ensure thorough staining.

Resuspend the fully stained cells in 20 mL of Sort Buffer, add 10 mL Sort Buffer to the FMO

Pass the fully stained solution through a 30-μm cell strainer into a new 50-ml conical, Pass the FMO through a 30-μm cell strainer into a new 50-ml conical as well.

Centrifuge at 400× G for 5 min at RT (full acceleration and full brake). Resuspend cells in up to 10e⁶/ml total cells (live and dead) in Sorting Buffer. Minimum volume is 300-μl.

Transfer to 15-ml conical tubes. Store the tubes in ice, covered with Aluminium foil until further use

Prepare 15-ml collection tubes for the sorted populations. Place 2-ml of Collection buffer (D-PBS with 2% hAB Serum) in the tubes. Store the collection tubes in ice until further use.

### Cell Counting and Viability

The procedures for obtaining cell and viability counts, using the Chemometec NC-200 Cell Counter are described in LAB-056

### FACS Sorting (FX500 Startup)

Turn on BSC. Turn on JUN-AIR vacuum pump. Turn on FX500 by pressing the Power/Standby button on the front of the instrument. Open Cell Sorter Software and run. Run automatic calibration using calibration reagents.

### Sample Collection

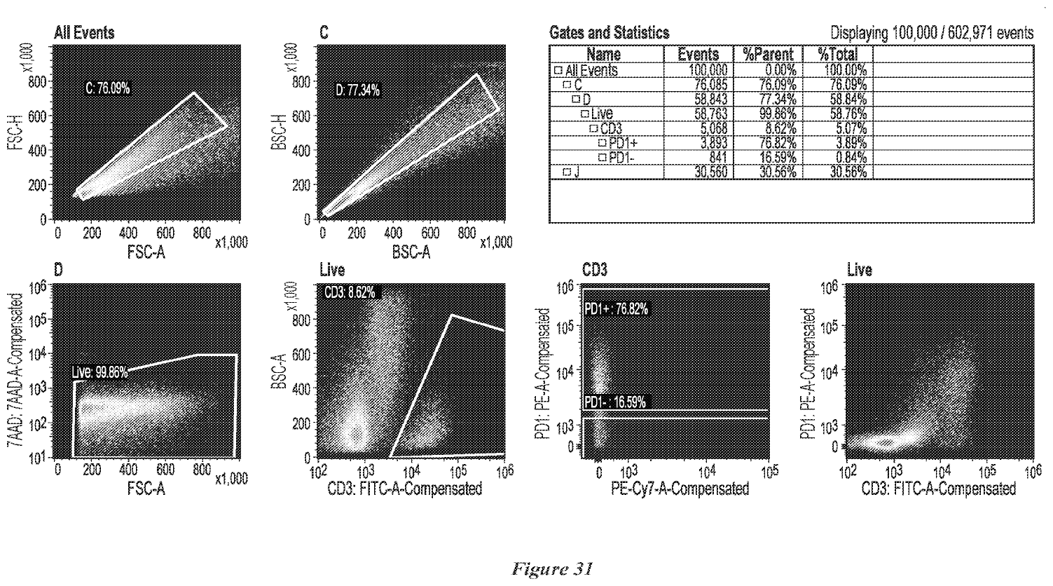

Verify that the sample and collection chambers are at 5° C. and that the agitate sample icon is selected and follow cytometer prompts for samples and compensation. Verify that the cell populations are gated correctly. See, FIG. 31.

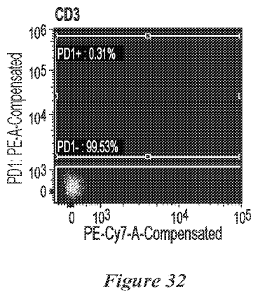

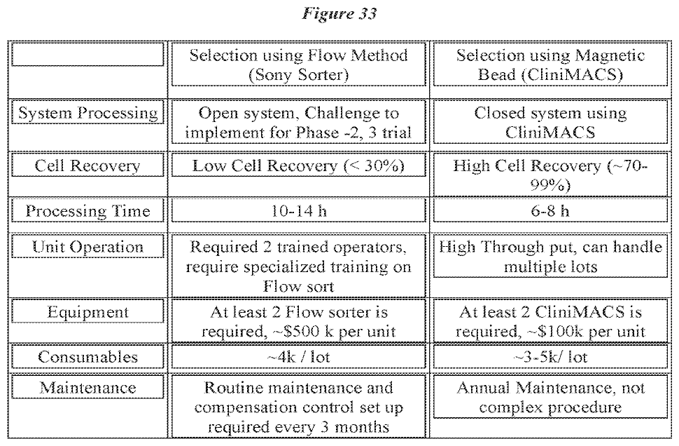

It may be necessary to adjust the BSC or FSC settings. Do not adjust the voltages for any other channels. Adjust the PD-1 gate if necessary. See, FIG. 32.

When the gates are satisfactory, Record as many events as possible (or 20,000 CD3 events maximum). You may set the sample pressure to 10 to speed up this collection.

Stop the collection and remove the tube. Load a 15-ml conical tube of sterile dH20 made previously onto the sample platform. Repeat until the CD3 gate is empty of events. Add the sample to be collected onto the loading platform. Verify that the settings are correct.

Load the 15-ml collection chamber block to the chamber. Load the collection tubes containing the collection buffer into the chamber block. Invert the capped tubes several times to coat the top of the tube with collection buffer. Label one tube with the sample name and a plus symbol. Remove cap and place this one into the left chamber. Label the second tube with the sample name and a negative symbol. Remove cap and place this one into the right chamber.

Run Load Collection icon and wait for the cells to appear on screen. About 15 seconds. Adjust the sample pressure so the total events per second are below 5.000. Click the start sort icon. Adjust the sample pressure to maintain a sorting efficiency of at least 85%. Record 50,000 CD3 events. The recording will stop automatically.

If there are over 4.5×10⁶ cells collected in either fraction, the collection tube(s) will need to be changed.

Continue sorting until all the sample is gone from the sample tube. Place the tubes on ice. Verify the selectivity percentages of the PD-1 fractions. Repeat process the negative selection sample. Export and save the data and shutdown flow cytometer.

### PHASE 1 (Feasibility Study)

### Day 0— REP1

### Media Preparation

Prepare or warm 500 mL of CM-1+6000 IU/mL IL-2.

### Defined Media Preparation

Prepare or warm 100 mL of CTS OpTimizer+3% SR and 6000 IU/mL IL-2. Remove 30 mL from 1L bottle of CTS OpTimizer. Add 30 mL of CTS Immune Cell SR, 1 mL of 50 mg/mL Gentamycin, 10 mL of 100X GlutaMAX, and 1 bottle of CTS Supplement (provided with CTS OpTimizer upon order). Store media at 4C until needed.

### PBMC Feeder Cell Preparation

Thaw an appropriate number of vials for REP 1 (10e6 PBMC will be needed for each condition, assume 60e6-80e6 PBMC per 1 mL vial). Place 40 mL of warm CM1+IL-2 in a 50 mL conical and pipette the 1 mL PBMC feeder vials into the conical. Pipette the thawed PBMC feeders up and down to thoroughly mix and perform 2 cell counts on the NC-200.

Calculate appropriate volume to transfer to each G-Rex 10M to transfer 10e6 PBMC. Add 3 uL of aCD3 (OKT-3) to each flask and place flasks into the incubator.

### Seeding TIL for REP1

Place 10% of the PD-1+sort volume (obtained by serological pipette) in to the G-Rex 10M flasks labelled PD-1+, PD-1+DM, and PD-1+Early REP. Fill PD-1+ and PD-1+Early REP to 100 mL with CM-1+IL-2, fill PD-1+DM to 100 mL with Defined Media+IL-2.

Calculate the proper volume of PD-1− to seed an equivalent number of PD-1−cells into the PD-1−G-Rex 10M flask. Fill flask to 100 mL with CM-1+IL-2. The CD3+bulk TIL control condition will add an equivalent number of CD3+ cells to PD-1+ cells that are in the other conditions. To obtain the proper volume of digest, follow the steps below. Calculate the CD3+TVC/mL in the digest by multiplying the digest TVC obtained in step 9.3.5 by the % CD3+ of live cells obtained from the sort report. (i.e. 10e6*10%=1e6). After obtaining this number, divide the number of PD-1+ cells seeded into each condition by this number. (i.e. 1e5/1e6=0.1 mL). Add this volume (0.1 mL) of digest to the bulk CD3+ TIL flask and fill to 100 mL with CM1+IL-2. Place all flasks into 37° C., 5% CO₂ incubator.

### Day 5 (Early REP) and Day 11— REP

### Media Preparation

Prepare or warm 250 mL of CM2+3000 IU/mL.

### Defined Media Preparation.

Prepare of warm 50 mL of Defined Media per section 9.12.1. (3000 IU/mL IL-2 instead of 6000 IU/mL).

### REP1 Harvest

Volume reduce flasks and place culture into appropriately labelled 50 mL conicals. Perform 2 cell counts on each samples on NC-200. Place 10% of the volume of each harvested REP1 (Maximum of 2e6 cells allowed into REP2) into their respective G-Rex 5M flasks and fill to 50 mL with warm CM2+IL-2 (PD-1+, PD-1+, PD-1+Early REP, PD-1-, and Bulk CD3+ TIL) or warm Defined Media (PD-1+DM).

### PBMC Feeder Cell Preparation

Thaw an appropriate number of vials for REP 1 (50e6 PBMC will be needed for each condition, assume 60e6-80e6 PBMC per 1 mL vial). Place 40 mL of warm CM1+IL-2 in a 50 mL conical and pipette the 1 mL PBMC feeder vials into the conical. Pipette the thawed PBMC feeders up and down to thoroughly mix and perform 2 cell counts on the NC-200. Calculate appropriate volume to transfer, and transfer feeders to new appropriately labelled G-Rex 5M to transfer 50e6 PBMC. Add 1.5 uL of aCD3 (OKT-3) to each flask and place flasks into the incubator. Place all flasks into 37C, 5% CO2 incubator.

### Day 12 (Early REP) and Day 16 Scale up

### Media Preparation

Prepare or warm 250 mL of CM4+3000 IU/mL.

### Defined Media Preparation

Prepare of warm 50 mL of Defined Media per section 9.12.1. (3000 IU/mL IL-2 instead of 6000 IU/mL).

### REP2 Harvest

Volume reduce flasks and place culture into appropriately labelled 50 mL conicals. Perform 2 cell counts on each samples on NC-200.

### Scale Up

Perform calculation to determine proper number of daughter flasks to scale up. TVC/10e6, rounded up, max of 5. Divide volume of harvested flask by number of daughter flasks, and place that volume back into the cultures respective G-Rex 5M flask. Fill flasks to 50 mL with warm CM4+IL-2 (PD-1+, PD-1+, PD-1+Early REP, PD-1-, and Bulk CD3+ TIL) or warm Defined Media (PD-1+DM).

### Day 17 (Early REP) and Day 22 Harvest

### Harvest

Volume reduce flasks and place culture into appropriately labelled 50 mL conicals.

Perform 2 cell counts on each samples on NC-200.

### Cryopreservation

Add PBS to the harvest product up to 50 mL and centrifuge at 400× G for 5 minutes at RT (full acceleration & full brake).

Resuspend each culture in 3 mL of cold CS-10 and aliquot into 1.8 mL cryovials.

Place cryovials into Mr. Frosty and place into −80C overnight. Place into LN2 storage the following day.

### PHASE 2

### Day 0— REP1

### Media Preparation

Prepare or warm 1L of CM-1+6000 IU/mL IL-2.

### PBMC Feeder Cell Preparation

Thaw an appropriate number of vials for REP 1 (100e6 PBMC will be needed for the full scale, and 10e6 will be needed for the Bulk CD3+Control, assume 60e6-80e6 PBMC per 1 mL vial).

Place 40 mL of warm CM1+IL-2 in a 50 mL conical and pipette the 1 mL PBMC feeder vials into the conical.

Pipette the thawed PBMC feeders up and down to thoroughly mix and perform 2 cell counts on the NC-200.

Calculate appropriate volume to transfer to the G-Rex 100M and G-Rex 10M to transfer 100e6 and 10e6 PBMC respectively.

Add 30 uL of aCD3 (OKT-3) to the G-Rex 100M and 3 uL into the G-rex 10M. Place flasks into the incubator

### Seeding TIL for REP1

Place all of the PD-1+sort into the G-Rex 100M.

The CD3+bulk TIL control condition will add an equivalent number of CD3+ cells as is PD-1+ cells in the full scale, in a 1/10 ratio. To obtain the proper volume of digest, follow the steps below.

Calculate the CD3+TVC/mL in the digest by multiplying the digest TVC obtained in step 9.3.5 by the % CD3+ of live cells obtained from the sort report. (i.e. 10e6*10%=1e6).

After obtaining this number, divide the number of PD-1+ cells seeded into the full scale condition by this number. (i.e. 1e5/1e6=0.1 mL).

Add this volume (0.1 mL) of digest to the bulk CD3+ TIL flask and fill to 100 mL with CM1+IL-2.

Place all flasks into 37C, 5% CO2 incubator

### Day 11, Day 16, Day 22

The full scale processes will be follow per IOVA manufacturing batch records.

The Bulk CD3+ TIL condition will be processed similarly to the steps described in Phase 1 for the small scale feasibility study.

Release Testing on the final product

For Phase-2 study, all the selected release testing will be performed except microbiological and endotoxin.

TABLE 55
Product Release Test Parameters

| Parameter | Test Method | Acceptance Criteria |
|---|---|---|
| Appearance | Visual inspection | Bag intact, no sign of clumps |
| Cell viability | Cell Counter | ≥70% |
| Total viable cells | Cell counter | 1.0 × 10⁹ to 150 × 10⁹ |
| Identity | Flow cytometry | ≥90% CD3⁺ CD45⁺ cells |
| Assay | IFN-γ | ≥500 pg/mL (Stimulated - |
| | | Unstimulated) |
| Microbiological | BacT Alert, | No growth |
| studies | aerobic and | |
| | anaerobic | |
| | Gram stain | Negative |
| | Real-time PCR for | Not detected |
| | mycoplasma | |
| Purity - | Limulus assay | ≤0.7 EU/mL |
| Endotoxin | | |

Extended Testing on the Final Produc

For Phase—1 and 2, additional characterization will be performed per research protocol (TP-18-015) and results will be recorded for information only.

Results and Acceptance Criteria.

TABLE 56
Harvest Product Testing and Expected results

| | | |
|---|---|---|
| Test Type | Method | Expected Result |

Flow Sorting

| | | |
|---|---|---|
| Post-sort Purity (% PD1+) | Flow Cytometry | ≥80% |

Release Testing

| | | |
|---|---|---|
| Appearance | Visual Inspection | Bag intact, no sign |
| | | of clumps |
| Cell viability | Fluorescence | ≥70% |
| Total Viable Cell Count | Fluorescence | 1 × 10e9 to 150 10e9 |
| Identity (% CD3/% CD45+) | Flow Cytometry | >90% CD3+ CD45+ |
| | | cells |
| Purity and Memory T cell | Flow Cytometry | Report results |
| subset Phenotype | (LAB-055) | |
| Activation and Exhaustion | Flow Cytometry | Report results |
| marker Phenotype | (LAB-061) | |
| Telomere length | Flow FISH | Report results |
| | (Attachment -1) | |
| IFNg(Stimulated - | Bead stimulation and ELISA | ≥500 pg/ml |
| Unstimulated) | | |
| Granzyme B | Bead stimulation and ELISA | Report results |
| | (LAB-064) | |
| CD107A | Mitogen stimulation and | Report results |
| | flow cytometry (LAB-061) | |
| TCR Vbeta Sequencing | Deep sequencing | Report results if |
| | (Irepertoire, Inc) | Available |
| Metabolite analysis | Cedex Biochemical analyzer | Report results |

### Example 8: Testing Anti-PD1 Coupled Microbeads for Positive Selection of PD-1+ TIL from Tumor Digest

### Background/Rationale for Magnetic Bead Selection

Antibody/magnetic bead Conjugation method. Selection of anti-PD-1+ TIL using anti-PD-1 magnetic beads

### Results:

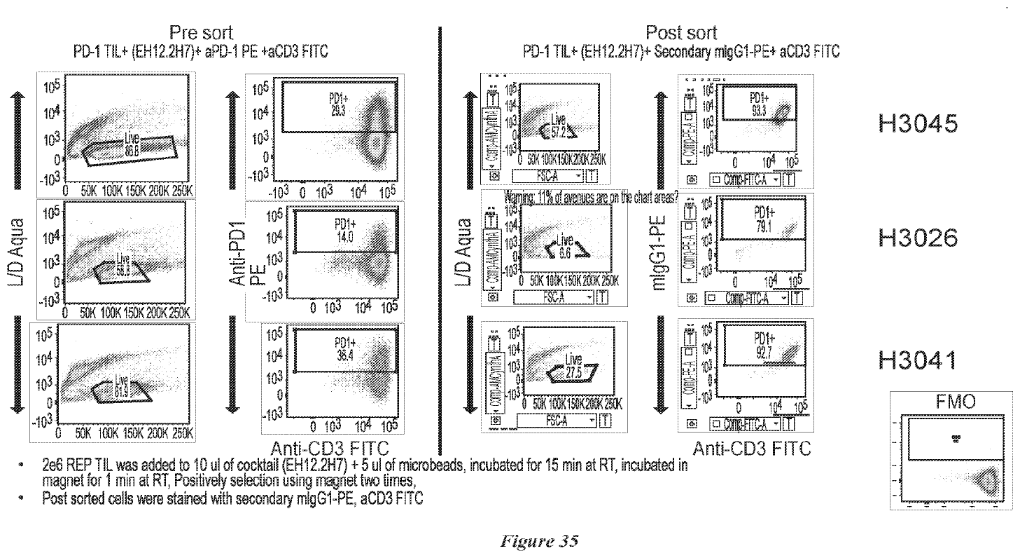

PD-1+ TIL can be selected from REP TIL using anti-PD-1 (EH12.H7) microbeads (FIG. 35).

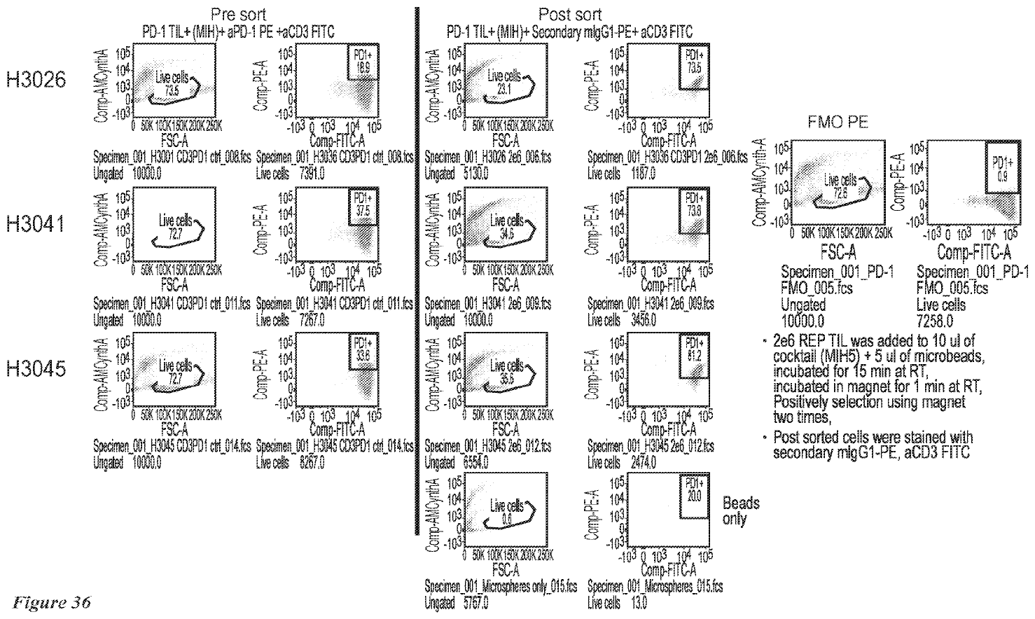

PD-1+ TIL can be selected from REP TIL using anti-PD-1 (M1H4) microbeads-(FIG. 36).

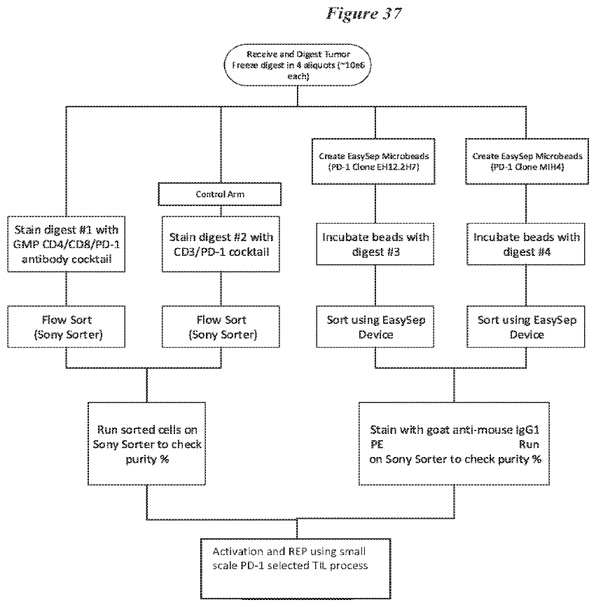

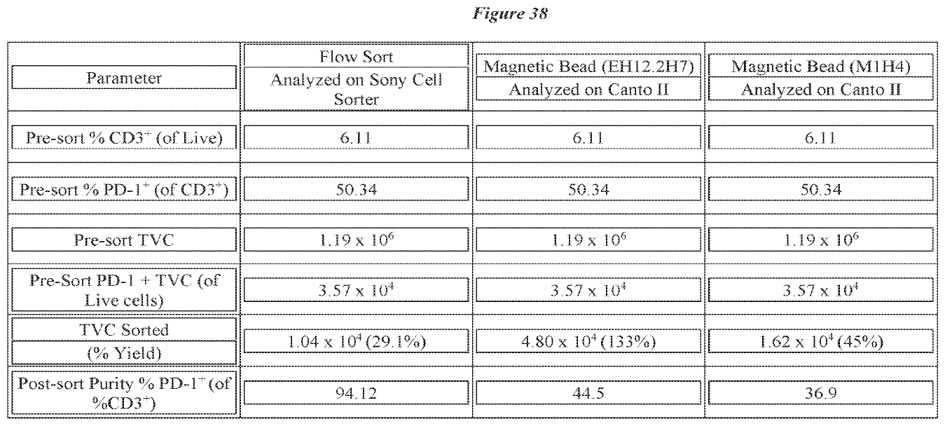

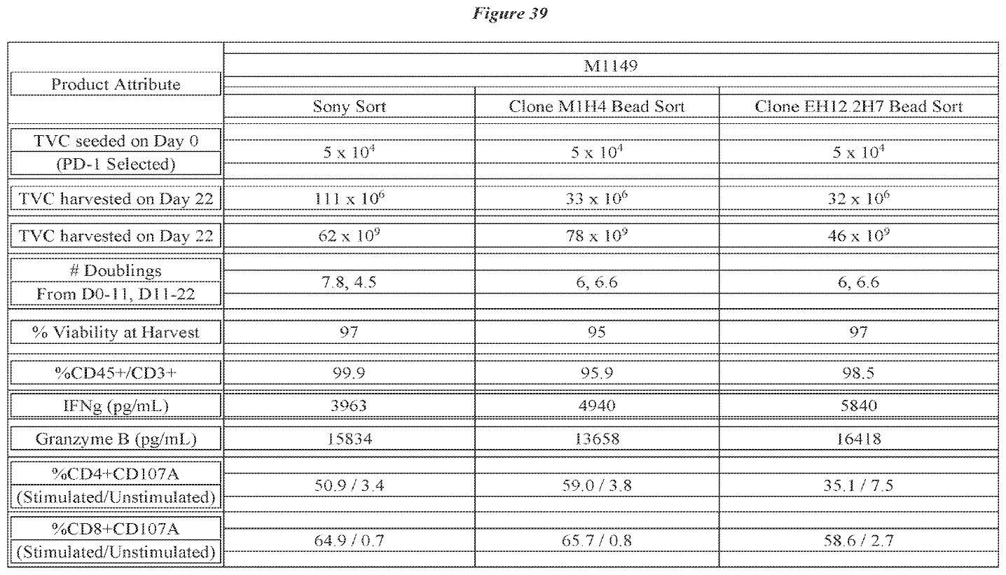

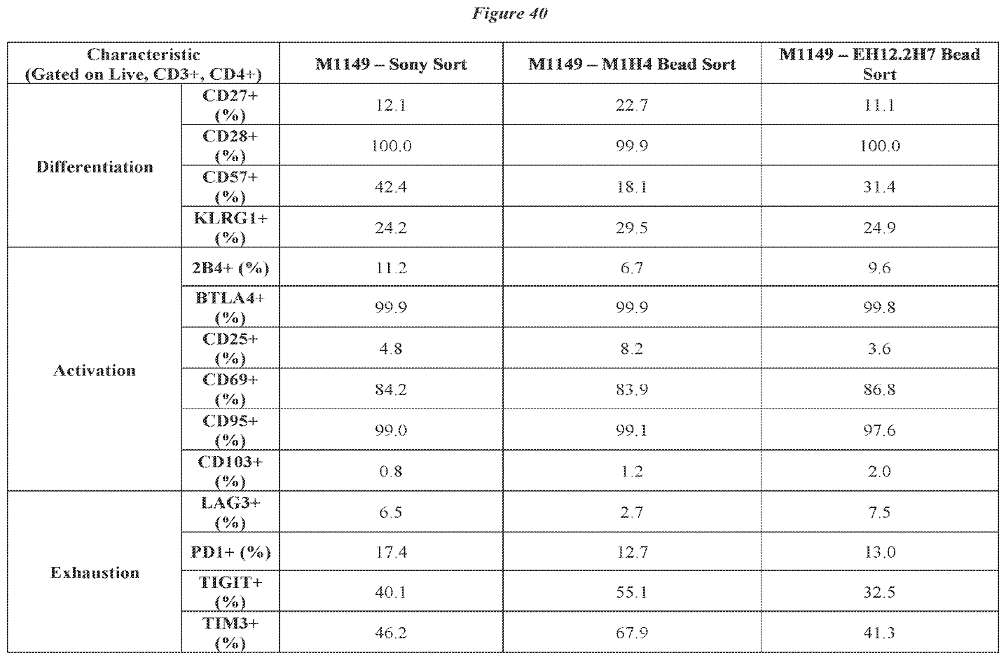

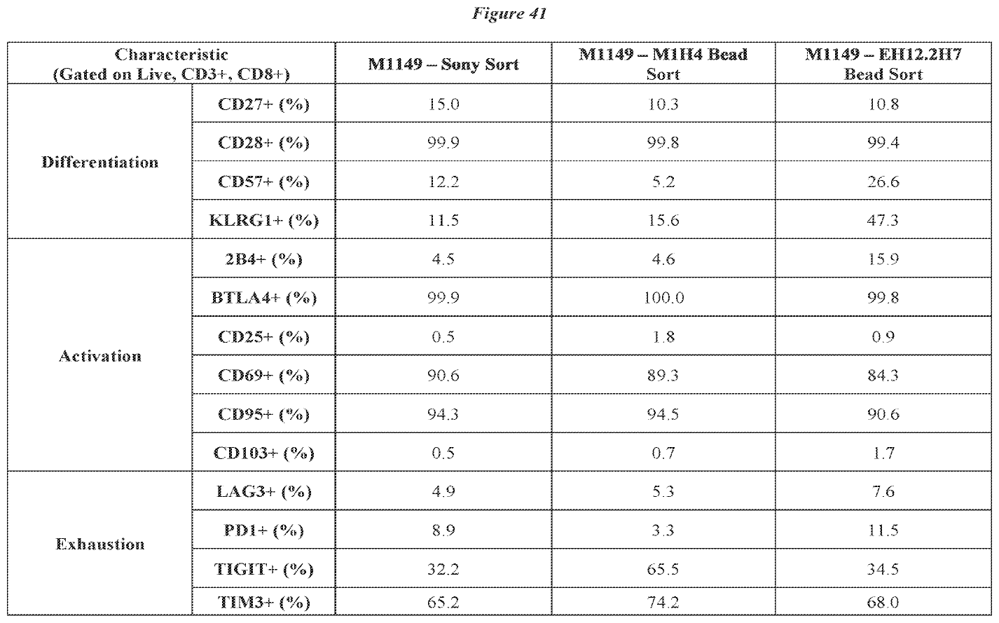

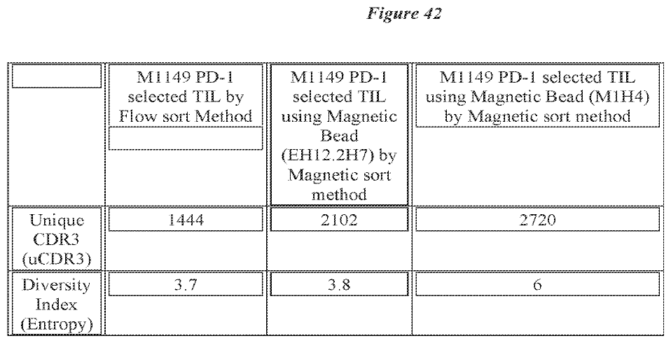

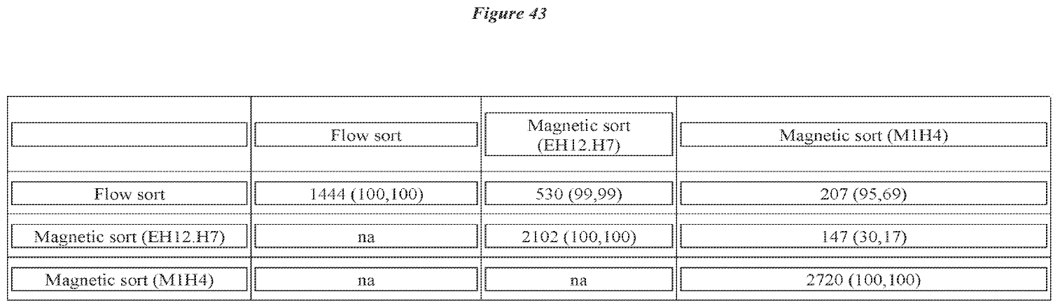

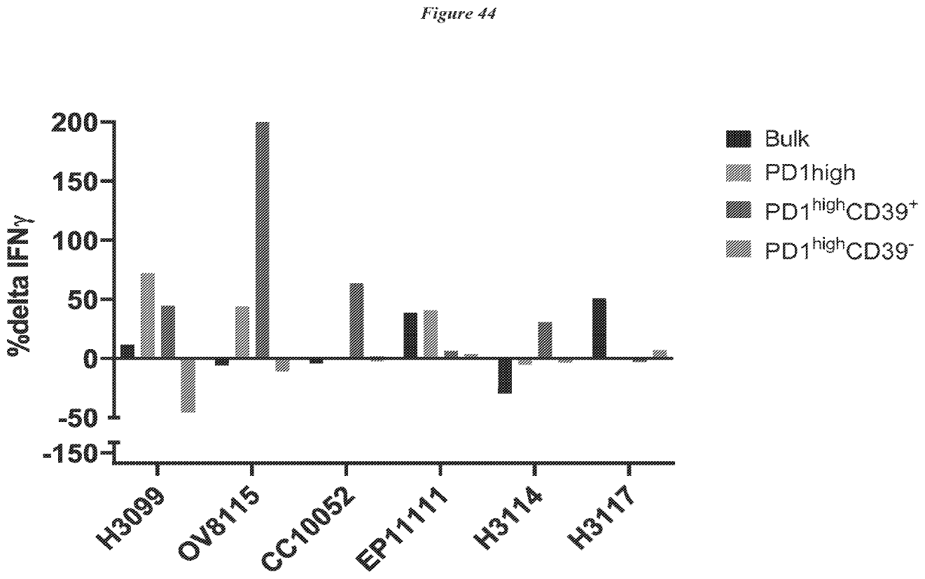

Head to Head study, selection and expansion of PD-1+ TIL using flow cytometry method or magnetic bead method (FIG. 37).

Pre/Post sort TVC on Day 0

Analyze the product attributes of the final product. Extended Phenotypic characterization of the final product.

Perform TCR Clonotype analysis.

Develop a magnetic bead sort protocol for PD-1 selection as an alternate embodiment to a flow sort.
Faster processing
Higher throughput
Less expensive
Less technical expertise required for operator (Flow sorting is a tedious process)
Enables closed system processing

PD-1 selected TIL using magnetic selection may not similar to PD-1⁺

### (Currently Flow Method)

### Mitigation

Consider alternate anti-PD-1 antibody from ThermoFisher (Clone MIH4) that will select only PD-1⁺.

In some embodiments, the StemCell Technologies's—EasySep “Do-It-Yourself” Positive Selection Kit was used for the conjugation process.

Antibodies chosen for conjugation:
CD279 (PD-1) Monoclonal Antibody (Clone—MIH4), eBioscience™
GoInVivo™ Purified anti-human CD279 (PD-1) Antibody (Clone— EH12.2H7) Conjugation Method

Add 15 ug of primary antibody to 100 uL of component A (proprietary blend of antibodies) and 100 uL of component B (proprietary blend of antibodies).
Incubate the mixture at 370C for overnight at 4C.
Next day, QS to 1 mL, with PBS.
Store at conjugated anti-PD-1 microbeads at 4C until further use.
Adjust Tumor digest concentration to 1e8 cells/mL (minimum volume of 100 uL).
Add 100 uL of conjugated anti-PD-1 microbeads to 1000 uL Tumor digest solution.
Incubate at RT for 15 minutes.
Vortex RapidSpheres for 30 seconds, Add RapidSpheres (50 uL/mL) to cells and incubate for 10 minutes at RT.
QS to 2.5 mL.
Incubate in EasySep magnets for 5-10 minutes.
Discard the supernatant.
Repeat step 4-6 for once.
Resuspend isolated cells in CM1.

A tumor (M1149) was processed and enzymatically digested using GMP enzymes (Collagenase, Dnase I, and Neutral Protease).

An equal part of tumor digest was sorted for PD-1+ TIL via the Sony Cell Sorter as well as Stem Cell anti-PD-1 magnetic beads made with two different antibody clones (EH12.2H7 and M1H4).

Post sort cell number yields using EH12.2H7 were higher than the flow sort method.

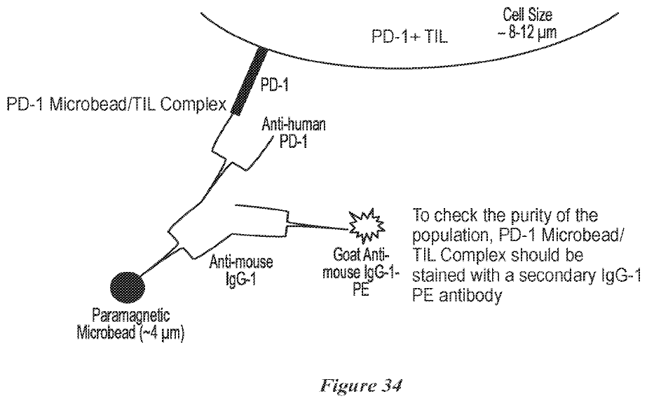

However, PD-1+ Purity using Flow sorting method were higher than the magnetic method. This could be due to the secondary antibody used in magnetic method to check the purity (see, FIG. 34).

Bead Kit used: EasySep™ Human “Do-It-Yourself” Positive Selection Kit II: Cat #17699.

Preliminary data suggest that magnetic bead based sorting can be used to select PD-1⁺ TIL.

Currently our staining method involve two steps. Staining of Tumor digest with Nivolumab, followed by anti-CD3 FITC and anti-IgG4 PE.

For the magnetic bead method, we propose to conjugate anti-IgG4 (HP-6023) with magnetic bead using.
Anti-Biotinated antibody with Biotinated micro beads from Miltenyi (CliniMACS).
Sepmag.
Dyna magnet.

### Embodiment 1

Tumor digest will be stained with Nivolumab followed by magnetic selection of PD-1+ TIL using bead coupled anti-IgG4 (HP6023).

### Embodiment 2

Need to analyze antibody (anti-PD-1) clones to select the PD-1⁺ TIL population. Analyzing M1H4 clone and new anti-PD-1 clone from (A17188B) Biolegend clone.

### Example 9: Exemplary Production of a Cryopreserved TIL Cell Therapy

This example describes an exemplary cGMP manufacture of TIL Cell Therapy Process in G-Rex Flasks according to current Good Tissue Practices and current Good Manufacturing Practices.

TABLE 57
Process Expansion Examplary Plan

| Estimated Day | | | | Estimated Total |
|---|---|---|---|---|
| (post-seed) | Activity | Target Criteria | Anticipated Vessels | Volume (mL) |
| 0 | Tumor Dissection | ≤50 desirable tumor fragments | G-Rex100MCS 1 flask | ≤1000 |
| | | per G-Rex100MCS | | |
| 11 | REP Seed | 5 − 200 × 10⁶ viable cells per | G-Rex500MCS 1 flasks | ≤5000 |
| | | G-Rex500MCS | | |
| 16 | REP Split | 1 × 10⁹ viable cells per | G-Rex500MCS ≤5 flasks | ≤25000 |
| | | G-Rex500MCS | | |
| 22 | Harvest | Total available cells | 3-4 CS-750 bags | ≤530 |

TABLE 58
Flask Volumes

| | Working |
|---|---|
| | Volume/Flask |
| Flask Type | (mL) |
| G-Rex100MCS | 1000 |
| G-Rex500MCS | 5000 |

### Process Information Primary

### Day 0 CM1 Media Preparation

In the BSC added reagents to RPMI 1640 Media bottle. Added the following reagents t Added per bottle: Heat Inactivated Human AB Serum (100.0 mL); GlutaMax (10.0 mL); Gentamicin sulfate, 50 mg/mL (1.0 mL); 2-mercaptoethanol (1.0 mL)

Removed unnecessary materials from BSC. Passed out media reagents from BSC, left Gentamicin Sulfate and HBSS in BSC for Formulated Wash Media preparation.

Thawed IL-2 aliquot. Thawed one 1.1 mL IL-2 aliquot (6×106 IU/mL) (BR71424) until all ice had melted. Recorded IL-2: Lot # and Expiry

Transferred IL-2 stock solution to media. In the BSC, transferred 1.0 mL of IL-2 stock solution to the CM1 Day 0 Media Bottle prepared. Added CM1 Day 0 Media 1 bottle and IL-2 (6×106 IU/mL) 1.0 mL.

Passed G-Rex100MCS into BSC. Aseptically passed G-Rex100MCS (W3013130) into the BSC.

Pumped all Complete CM1 Day 0 Media into G-Rex100MCS flask. Tissue Fragments Conical or GRex100MCS

### Day 0 Tumor Wash Media Preparation

In the BSC, added 5.0 mL Gentamicin (W3009832 or W3012735) to 1×500 mL HBSS Media (W3013128) bottle. Added per bottle: HBSS (500.0 mL); Gentamicin sulfate, 50 mg/ml (5.0 mL). Filtered HBSS containing gentamicin prepared through a 1L 0.22-micron filter unit (W1218810).

### Day 0 Tumor Processing

Obtained Tumor. Obtained tumor specimen from QAR and transferred into suite at 2-8° C. immediately for processing.

Aliquoted Tumor Wash Media.

Tumor Wash 1 Using 8” forceps (W3009771), removed the tumor from the specimen bottle and transferred to the “Wash 1” dish prepared. Followed by Tumor Wash 2 and Tumor Wash 3.

Measured Tumor. Assessed Tumor. Assessed whether >30% of entire tumor area observed to be necrotic and/or fatty tissue. If applicable: Clean-Up Dissection. If tumor was large and >30% of tissue exterior was observed to be necrotic/fatty, performed “clean up dissection” by removing necrotic/fatty tissue while preserving tumor inner structure using a combination of scalpel and/or forceps.

Dissect Tumor Using a combination of scalpel and/or forceps, cut the tumor specimen into even, appropriately sized fragments (up to 6 intermediate fragments). Transferred intermediate tumor fragments. Dissected Tumor Fragments into pieces approximately 3×3×3 mm in size. Stored Intermediate Fragments to Prevent Drying.

Repeated Intermediate Fragment Dissection. Determined number of pieces collected. If desirable tissue remains, selected additional Favorable Tumor Pieces from the “favorable intermediate fragments” 6-well plate to fill the drops for a maximum of 50 pieces.

Prepared Conical Tube. Transferred Tumor Pieces to 50 mL Conical Tube. Prepared BSC for G-REX100MCS. Removed G-REX100MCS from Incubator. Aseptically passed G-Rex100MCS flask into the BSC. Added tumor fragments to G-Rex100MCS flask. Evenly distributed pieces.

Incubated G-Rex100MCS at the following parameters: Incubated G-Rex flask: Temperature LED Display: 37.0+2.0° C.; CO2 Percentage: 5.0+1.5% CO2. Calculations: Time of incubation; lower limite=time of incubation+252 hours; upper limit=time of incubation+276 hours.

After process was complete, discarded any remaining warmed media and thawed aliquots of IL-2.

### Day 11— Media Preparation

Monitored Incubator. Monitored Incubator. Incubator parameters: Temperature LED Display: 37.0+2.0° C.; CO2 Percentage: 5.0+1.5% CO2.

Warmed 3×1000 mL RPMI 1640 Media (W3013112) bottles and 3×1000 mL AIM-V (W3009501) bottles in an incubator for ≥30 minutes. Removed RPMI 1640 Media from incubator. Prepared RPMI 1640 Media. Filter Media. Thawed 3×1.1 mL aliquots of IL-2 (6×106 IU/mL) (BR71424). Removed AIM-V Media from the incubator. Add IL-2 to AIM-V. Aseptically transferred a 10 L Labtainer Bag and a repeater pump transferr set into the BSC.

Prepared 10 L Labtainer media bag. Prepared Baxa pump. Prepared 10 L Labtainer media bag. Pumped media into 10 L Labtainer. Removed pumpmatic from Labtainer bag.

Mixed media. Gently massaged the bag to mix. Sample media per sample plan. Removed 20.0 mL of media and place in a 50 mL conical tube. Prepared Cell Count Dilution Tubes In the BSC, added 4.5 mL of AIM-V Media that had been labelled with “For Cell Count Dilutions” and lot number to four 15 mL conical tubes. Transferred reagents from the BSC to 2-8° C. Prepared 1L Transfer Pack. Outside of the BSC weld (per Process Note 5.11) a 1L Transfer Pack to the transfer set attached to the “Complete CM2 Day 11 Media” bag prepared. Prepared feeder cell transfer pack. Incubated Complete CM2 Day 11 Media.

### Day 11—TIL Harvest

Preprocessing table. Incubator parameters: Temperature LED Display: 37.0±2.0° C.; CO2 Percentage: 5.0±1.5% CO2. Removed G-Rex100MCS from incubator. Prepared 300 mL Transfer Pack. Welded transfer packs to G-Rex100MCS.

Prepare flask for TIL Harvest and nitiation of TIL Harvest. TIL Harvested. Using the GatheRex, transferred the cell suspension through the blood filter into the 300 mL transfer pack. Inspect membrane for adherent cells.

Rinsed flask membrane. Closed clamps on G-Rex100MCS. Ensured all clamps are closed. Heat sealed the TIL and the “Supernatant” transfer pack. Calculated volume of TIL suspension. Prepared Supernatant Transfer Pack for Sampling.

Pulled Bac-T Sample. In the BSC, draw up approximately 20.0 mL of supernatant from the 1L “Supernatant” transfer pack and dispense into a sterile 50 mL conical tube.

Inoculated BacT per Sample Plan. Removed a 1.0 mL sample from the 50 mL conical labeled BacT prepared using an appropriately sized syringe and inoculated the anaerobic bottle.

Incubated TIL. Placed TIL Transfer Pack in incubator until needed. Performed cell counts and calculations. Determined the Average of Viable Cell Concentration and Viability of the cell counts performed. Viability÷2. Viable Cell Concentration÷2. Determined Upper and Lower Limit for counts. Lower Limit: Average of Viable Cell Concentration×0.9. Upper Limit: Average of Viable Cell Concentration×1.1. Confirmed both counts within acceptable limits. Determined an average Viable Cell Concentration from all four counts performed.

Adjusted Volume of TIL Suspension Calculate the adjusted volume of TIL suspension after removal of cell count samples. Total TIL Cell Volume (A). Volume of Cell Count Sample Removed (4.0 ml) (B) Adjusted Total TIL Cell Volume C=A−B.

Calculated Total Viable TIL Cells. Average Viable Cell Concentration*: Total Volume; Total Viable Cells: C=A×B.

Calculation for flow cytometry: if the Total Viable TIL Cell count from was ≥4.0×10⁷, calculated the volume to obtain 1.0×10⁷ cells for the flow cytometry sample.

Total viable cells required for flow cytometry: 1.0×10⁷ cells. Volume of cells required for flow cytometry: Viable cell concentration divided by 1.0×10⁷ cells A.

Calculated the volume of TIL suspension equal to 2.0×10⁸ viable cells. As needed, calculated the excess volume of TIL cells to remove and removed excess TIL and placed TIL in incubator as needed. Calculated total excess TIL removed, as needed.

Calculated amount of CS-10 media to add to excess TIL cells with the target cell concentration for freezing is 1.0×10⁸ cells/ml. Centrifuged excess TILs, as needed. Observed conical tube and added CS-10.

Filled Vials. Aliquoted 1.0 mL cell suspension, into appropriately sized cryovials. Aliquoted residual volume into appropriately sized cryovial. If volume is ≤0.5 mL, add CS10 to vial until volume is 0.5 mL.

TIL Cryopreservation of Sample

Calculated the volume of cells required to obtain 1×10⁷ cells for cryopreservation. Removed sample for Cryopreservation. Placed TIL in Incubator. Cryopreservation of sample.

Observed conical tube and added CS-10 slowly and record volume of 0.5 mL of CS10 added.

### Day 11—Feeder Cells

Obtained feeder cells. Obtained 3 bags of feeder cells with at least two different lot numbers from LN2 freezer. Kept cells on dry ice until ready to thaw. Prepared waterbath or Cryotherm. Thawed Feeder Cells at 37.0±2.0° C. water bath or cytotherm for ˜3-5 minutes or until ice has just disappeared. Removed media from incubator. Pooled thawed feeder cells. Added feeder cells to transfer pack. Dispensed the feeder cells from the syringe into the transfer pack. Mixed pooled feeder cells and labeled transfer pack.

Calculated Total Volume of Feeder Cell Suspension in Transfer Pack

Removed cell count samples. Using a separate 3 mL syringe for each sample, pulled 4×1.0 mL cell count samples from Feeder Cell Suspension Transfer Pack using the needless injection port. Aliquoted each sample into the cryovials labeled. Performed Cell Counts and Determine Multiplication Factor Selected protocols and entered multiplication factors. Determined the Average of Viable Cell Concentration and Viability of the cell counts performed. Determined Upper and Lower Limit for counts and confirm within limits.

Adjusted Volume of Feeder Cell Suspension. Calculated the adjusted volume of Feeder Cell suspension after removal of cell count samples. Calculated Total Viable Feeder Cells. Obtained additional Feeder Cells as needed. Thawed Additional Feeder Cells as needed. Placed the 4th Feeder Cell bag into a zip top bag and thaw in a 37.0±2.0° C. water bath or cytotherm for ˜3-5 minutes and pooled additional feeder cells. Measured Volume. Measured the volume of the feeder cells in the syringe and recorded below (B). Calculated the new total volume of feeder cells. Added Feeder Cells to Transfer Pack.

Prepared dilutions as needed, adding 4.5 mL of AIM-V Media to four 15 mL conical tubes. Prepared cell counts. Using a separate 3mLsyringe for each sample, removed 4×1.0 mL cell count samples from Feeder Cell Suspension transfer pack, using the needless injection port. Performed cell counts and calculations. Determined an average Viable Cell Concentration from all four counts performed. Adjusted Volume of Feeder Cell suspension and calculated the adjusted volume of Feeder Cell suspension after removal of cell count samples. Total Feeder Cell Volume minutes 4.0 mL removed. Calculated the volume of Feeder Cell Suspension that was required to obtain 5×10⁹ viable feeder cells. Calculated excess feeder cell volume. Calculated the volume of excess feeder cells to remove. Removed excess feeder cells.

Using a 1.0 mL syringe and 16 G needle, drew up 0.15 mL of OKT3 and added OKT3. Heat sealed the Feeder Cell Suspension transfer pack.

### Day 11 G-Rex Fill and Seed

Set up G-Rex500MCS. Removed “Complete CM2 Day 11 Media”, from incubator and pumped media into G-Rex500MCS. Pumped 4.5 L of media into the G-Rex500MCS, filling to the line marked on the flask. Heat sealed and incubated flask as needed. Welded the Feeder Cell suspension transfer pack to the G-Rex500MCS. Added Feeder Cells to G-Rex500MCS. Heat sealed. Welded the TIL Suspension transfer pack to the flask. Added TIL to G-Rex500MCS. Heat sealed. Incubated G-Rex500MCS at 37.0±2.0° C., CO2 Percentage: 5.0±1.5% CO2.

Calculated incubation window. Performed calculations to determine the proper time to remove G-Rex500MCS from incubator on Day 16. Lower limit: Time of incubation+108 hours. Upper limit: Time of incubation+132 hours.

### Day 11 Excess TIL Cryopreservation

Applicable: Froze Excess TIL Vials. Verified the CRF has been set up prior to freeze. Perform Cryopreservation. Transferred vials from Controlled Rate Freezer to the appropriate storage. Upon completion of freeze, transfer vials from CRF to the appropriate storage container. Transferred vials to appropriate storage. Recorded storage location in LN2.

### Day 16 Media Preparation

Pre-warmed AIM-V Media. Calculated time Media was warmed for media bags 1, 2, and 3. Ensured all bags have been warmed for a duration between 12 and 24 hours. Setup 10 L Labtainer for Supernatant. Attached the larger diameter end of a fluid pump transfer set to one of the female ports of a 10 L Labtainer bag using the Luer connectors. Setup 10 L Labtainer for Supernatant and label. Setup 10 L Labtainer for Supernatant. Ensure all clamps were closed prior to removing from the BSC. NOTE: Supernatant bag was used during TIL Harvest, which may be performed concurrently with media preparation.

Thawed IL-2. Thawed 5×1.1 mL aliquots of IL-2 (6×10⁶ IU/mL) (BR71424) per bag of CTS AIM V media until all ice had melted. Aliquoted 100.0 mL GlutaMax. Added IL-2 to GlutaMax. Prepared CTS AIM V media bag for formulation. Prepared CTS AIM V media bag for formulation. Stage Baxa Pump. Prepared to formulate media. Pumped GlutaMax +IL-2 into bag. Monitored parameters: Temperature LED Display: 37.0+2.0° C., CO2 Percentage: 5.0±1.5% CO2. Warmed Complete CM4 Day 16 Media. Prepared Dilutions.

### Day 16 REP Spilt

Monitored Incubator parameters: Temperature LED Display: 37.0±2.0° C., CO2 Percentage: 5.0±1.5% CO2. Removed G-Rex500MCS from the incubator. Prepared and labeled 1L Transfer Pack as TIL Suspension and weighed 1L.

Volume Reduction of G-Rex500MCS. Transferred ˜4.5 L of culture supernatant from the G-Rex500MCS to the 10 L Labtainer.

Prepared flask for TIL Harvest. After removal of the supernatant, closed all clamps to the red line.

Initiation of TIL Harvest. Vigorously tap flask and swirl media to release cellsensure all cells have detached.

TIL Harvest. Released all clamps leading to the TIL suspension transfer pack. Using the GatheRex transferred the cell suspension into the TIL Suspension transfer pack. NOTE: Be sure to maintain the tilted edge until all cells and media are collected. Inspected membrane for adherent cells. Rinsed flask membrane. Closed clamps on G-Rex500MCS. Heat sealed the Transfer Pack containing the TIL. Heat sealed the 10 L Labtainer containing the supernatant. Recorded weight of Transfer Pack with cell suspension and calculate the volume suspension. Prepared transfer pack for sample removal. Removed testing samples from cell supernatant.

Sterility & BacT Testing Sampling: removed a 1.0 mL sample from the 15 mL conical labeled BacT prepared. Removed Cell Count Samples. In the BSC, using separate 3 mL syringes for each sample, removed 4×1.0 mL cell count samples from “TIL Suspension” transfer pack.

Removed Mycoplasma Samples. Using a 3 mL syringe, removed 1.0 mL from TIL Suspension transfer pack and place into 15 mL conical labeled “Mycoplasma diluent” prepared.

Prepared Transfer Pack for Seeding. Placed TIL in Incubator. Removed cell suspension from the BSC and place in incubator until needed. Performed cell counts and calculations. Diluted cell count samples initially by adding 0.5 mL of cell suspension into 4.5 mL of AIM-V media prepared which gave a 1:10 dilution. Determined the Average of Viable Cell Concentration and Viability of the cell counts performed. Determined Upper and Lower Limit for counts. NOTE: Dilution may be adjusted according based off the expected concentration of cells. Determined an average Viable Cell Concentration from all four counts performed. Adjusted Volume of TIL Suspension. Calculated the adjusted volume of TIL suspension after removal of cell count samples. Total TIL Cell Volume minus 5.0 mL removed for testing.

Calculated Total Viable TIL Cells. Calculated the total number of flasks to seed. NOTE: The maximum number of G-Rex500MCS flasks to seed was five. If the calculated number of flasks to seed exceeded five, only five were seeded USING THE ENTIRE VOLUME OF CELL SUSPENSION AVAILABLE.

Calculate number of flasks for subculture. Calculated the number of media bags required in addition to the bag prepared. Prepared one 10 L bag of “CM4 Day 16 Media” for every two G-Rex-500M flask needed as calculated. Proceeded to seed the first GREX-500M flask(s) while additional media is prepared and warmed. Prepared and warmed the calculated number of additional media bags determined. Filled G-Rex500MCS. Prepared to pump media and pumped 4.5 L of media into G-Rex500MCS. Heat Sealed. Repeated Fill. Incubated flask. Calculated the target volume of TIL suspension to add to the new G-Rex500MCS flasks. If the calculated number of flasks exceeds five only five will be seeded, USING THE ENTIRE VOLUME OF CELL SUSPENSION. Prepared Flasks for Seeding. Removed G-Rex500MCS from the incubator. Prepared G-Rex500MCS for pumping. Closed all clamps on except large filter line. Removed TIL from incubator. Prepared cell suspension for seeding. Sterile welded (per Process Note 5.11) “TIL Suspension” transfer pack to pump inlet line. Placed TIL suspension bag on a scale.

Seeded flask with TIL Suspension. Pump the volume of TIL suspension calculated into flask. Heat sealed. Filled remaining flasks.

Monitored Incubator. Incubator parameters: Temperature LED Display: 37.0±2.0° C., CO2 Percentage: 5.0±1.5% CO2. Incubated Flasks.

Determined the time range to remove G-Rex500MCS from incubator on Day 22.

### Day 22 Wash Buffer Preparation

Prepared 10 L Labtainer Bag. In BSC, attach a 4″ plasma transfer set to a 10 L Labtainer Bag via luer connection. Prepared 10 L Labtainer Bag. Closed all clamps before transferring out of the BSC. NOTE: Prepared one 10 L Labtainer Bag for every two G-Rex500MCS flasks to be harvested. Pumped Plasmalyte into 3000 mL bag and removed air from 3000 mL Origen bag by reversing the pump and manipulating the position of the bag. Added Human Albumin 25% to 3000 mL Bag. Obtain a final volumeof 120.0 mL of Human Albumin 25%.

Prepared IL-2 Diluent. Using a 10 mL syringe, removed 5.0 mL of LOVO Wash Buffer using the needleless injection port on the LOVO Wash Buffer bag. Dispensed LOVO wash buffer into a 50 mL conical tube.

CRF Blank Bag LOVO Wash Buffer Aliquotted. Using a 100 mL syringe, drew up 70.0 mL of LOVO Wash Buffer from the needleless injection port.

Thawed IL-2. Thawed one 1.1 mL of IL-2 (6×106 IU/mL)), until all ice has melted. IL-2 Preparation. Added 504 IL-2 stock (6×106 IU/mL) to the 50 mL conical tube labeled “IL-2 Diluent.”

Cryopreservation Prep. Placed 5 cryo-cassettes at 2-8° C. to precondition them for final product cryopreservation.

Prepared Cell Count Dilutions. In the BSC, added 4.5 mL of AIM-V Media that has been labelled with lot number and “For Cell Count Dilutions” to 4 separate 15 mL conical tubes. Prepared Cell Counts. Labeled 4 cryovials with vial number (1-4). Kept vials under BSC to be used.

### Day 22 TIL Harvest

Monitored Incubator. Incubator Parameters Temperature LED display: 37±2.0° C., CO2 Percentage: 5%±1.5%. Removed G-Rex500MCS Flasks from Incubator. Prepared TIL collection bag and labeled. Sealed off extra connections. Volume Reduction: Transferred ˜4.5 L of supernatant from the G-Rex500MCS to the Supernatant bag.

Prepared flask for TIL Harvest. Initiated collection of TIL. Vigorously tap flask and swirl media to release cells. Ensure all cells have detached. Initiated collection of TIL. Released all clamps leading to the TIL suspension collection bag. TIL Harvest. Using the GatheRex, transferred the TIL suspension into the 3000 mL collection bag. Inspect membrane for adherent cells. Rinsed flask membrane. Closed clamps on G-Rex500MCS and ensured all clamps are closed. Transferred cell suspension into LOVO source bag. Closed all clamps. Heat Sealed. Removed 4×1.0 mL Cell Counts Samples

Performed Cell Counts. Performed cell counts and calculations utilizing NC-200 and Process Note 5.14. Diluted cell count samples initially by adding 0.5 mL of cell suspension into 4.5 mL of AIM-V media prepared. This gave a 1:10 dilution. Determined the Average Viability, Viable Cell Concentration, and Total Nucleated Cell concentration of the cell counts performed. Determined Upper and Lower Limit for counts. Determined the Average Viability, Viable Cell Concentration, and Total Nucleated Cell concentration of the cell counts performed. Weighed LOVO Source Bag. Calculated Total Viable TIL Cells. Calculated Total Nucleated Cells.

Prepared Mycoplasma Diluent. Removed 10.0 mL from one supernatant bag via luer sample port and placed in a 15 mL conical.

### LOVO

Performed “TIL G-Rex Harvest” protocol and determined the final product target volume. Loaded disposable kit. Removed filtrate bag. Entered Filtrate capacity. Placed Filtrate container on benchtop. Attached PlasmaLyte. Verified that the PlasmaLyte was attached and observed that the PlasmaLyte is moving. Attached Source container to tubing and verified Source container was attached. Confirmed PlasmaLyte was moving.

### Final Formulation and Fill

Target volume/bag calculation. Calculated volume of CS-10 and LOVO wash buffer to formulate blank bag. Prepared CRF Blank.

Calculated the volume of IL-2 to add to the Final Product. Final IL-2 Concentration desired (IU/mL) −300 IU/mL. IL-2 working stock: 6×10⁴ IU/mL. Assembled Connect apparatus. Sterile welded a 4S-4M60 to a CC2 Cell Connection. Sterile welded (per Process Note 5.11) the CS750 Cryobags to the harness prepared. Welded CS-10 bags to spikes of the 4S-4M60. Prepared TIL with IL-2. Using an appropriately sized syringe, removed amount of IL-2 determined from the “1L-2 6×10⁴” aliquot. Labeled Formulated TIL Bag. Added the Formulated TIL bag to the apparatus. Added CS10. Switched Syringes. Drew ˜10 mL of air into a 100 mL syringe and replaced the 60 mL syringe on the apparatus. Added CS10. Prepared CS-750 bags. Dispensed cells.

Removed air from final product bags and take retain. Once the last final product bag was filled, closed all clamps. Drew 10 mL of air into a new 100 mL syringe and replace the syringe on the apparatus. Dispensed retain into a 50 mL conical tube and label tube as “Retain” and lot number. Repeat air removal step for each bag.

Prepared final product for cryopreservation, including visual inspection. Held the cryobags on cold pack or at 2-8° C. until cryopreservation.

Removed Cell Count Sample. Using an appropriately sized pipette, remove 2.0 mL of retain and place in a 15 mL conical tube to be used for cell counts. Performed cell counts and calculations. NOTE: Diluted only one sample to appropriate dilution to verify dilution is sufficient. Diluted additional samples to appropriate dilution factor and proceed with counts. Determined the Average of Viable Cell Concentration and Viability of the cell counts performed. Determined Upper and Lower Limit for counts. NOTE: Dilution may be adjusted according based off the expected concentration of cells. Determined the Average of Viable Cell Concentration and Viability. Determined Upper and Lower Limit for counts. Calculated IFN-γ. Heat Sealed Final Product Bags.

Labeled and Collected Samples per exemplary Sample Plan below.

TABLE 59
Sample Plan

| | | Sample | |
|---|---|---|---|
| | | Volume to | |
| | Number of | Add to | Container |
| Sample | Containers | Each | Type |
| *Mycoplasma | 1 | 1.0 mL | 15 mL Conical |
| Endotoxin | 2 | 1.0 mL | 2 mL Cryovial |
| Gram Stain | 1 | 1.0 mL | 2 mL Cryovial |
| IFN-g | 1 | 1.0 mL | 2 mL Cryovial |
| Flow | 1 | 1.0 mL | 2 mL Cryovial |
| Cytometry | | | |
| **Bac-T | 2 | 1.0 mL | Bac-T Bottle |
| Sterility | | | |
| QC Retain | 4 | 1.0 mL | 2 mL Cryovial |
| Satellite Vials | 10 | 0.5 mL | 2 mL Cryovial |

Sterility & BacT. Testing Sampling. In the BSC, remove a 1.0 mL sample from the retained cell suspension collected using an appropriately sized syringe and inoculate the anaerobic bottle. Repeat the above for the aerobic bottle

### Final Product Cryopreservation

Prepared Controlled Rate Freezer. Verified the CRF had been set up. Set up CRF probes. Placed final product and samples in CRF. Determined the time needed to reach 4° C.±1.5° C. and proceed with the CRF run. CRF Completed and Stored. Stopped the CRF after the completion of the run. Remove cassettes and vials from CRF. Transferred cassettes and vials to vapor phase LN2 for storage. Recorded storage location

### Post Processing Summary

### Post-Processing: Final Drug Product

(Day 22) Determination of CD3+ Cells on Day 22 REP by Flow Cytometry

(Day 22) Gram Staining Method (GMP)

(Day 22) Bacterial Endotoxin Test by Gel Clot LAL Assay (GMP)

(Day 16) BacT Sterility Assay (GMP)

(Day 16) Mycoplasma DNA Detection by TD-PCR (GMP)

Acceptable Appearance Attributes

(Day 22) BacT Sterility Assay (GMP)(Day 22)

(Day 22) IFN-gamma Assay

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method for expanding tumor infiltrating lymphocytes (TILs) into a therapeutic population of TILs comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs;
(d) performing a rapid second expansion by supplementing the cell culture medium of the second population of TILs with additional IL-2, OKT-3, and APCs, to produce a third population of TILs, wherein the number of APCs added in the rapid second expansion is at least twice the number of APCs added in step (c), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(e) harvesting the therapeutic population of TILs obtained from step (d); and
(f) transferring the harvested TIL population from step (e) to an infusion bag.

2. A method for expanding tumor infiltrating lymphocytes (TILs) into a therapeutic population of TILs comprising:
f) obtaining and/or receiving a first population of TILs from a tumor resected from a subject by processing a tumor sample obtained from the subject into multiple tumor fragments;
g) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
h) performing a priming first expansion by culturing the PD-1, CD39, CD38, CD103, CD101, LAG3, TIM3 and/or TIGIT enriched TIL population in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs;
i) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and
j) harvesting the therapeutic population of TILs obtained from step (d).

3. The method of claim 2, wherein in step (c) the cell culture medium further comprises antigen-presenting cells (APCs), and wherein the number of APCs in the culture medium in step (d) is greater than the number of APCs in the culture medium in step (c).

4. The method of claim 2, wherein in step (c) the cell culture medium further comprises antigen-presenting cells (APCs), and wherein the number of APCs in the culture medium in step (d) is equal to the number of APCs in the culture medium in step (c).

5. The method of claim 1 or 2, wherein said PD-1, CD39, CD38, CD103, CD101, LAG3, TIM3 and/or TIGIT positive TILs are PD-1high, CD39high/lo, CD38lo, CD103high/lo, CD101lo, LAG3high, TIM3high and/or TIGIThigh TILs.

6. A method for expanding tumor infiltrating lymphocytes (TILs) into a therapeutic population of TILs comprising:
(a) performing a priming first expansion by culturing a first population of TILs which have been selected to be PD-1, CD39, CD38, CD103, CD101, LAG3, TIM3 and/or TIGIT positive, said first population of TILs obtainable by processing a tumor sample from a subject by tumor digestion and selecting for the PD-1, CD39, CD38, CD103, CD101, LAG3, TIM3 and/or TIGIT positive TILs, in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7, 8, 9, 10, or 11 days to obtain the second population of TILs, wherein the second population of TILs is greater in number than the first population of TILs;
(b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area; and
(c) harvesting the therapeutic population of TILs obtained from step (b).

7. A method for expanding tumor infiltrating lymphocytes (TILs) into a therapeutic population of TILs comprising:
(a) performing a priming first expansion of a first population of TILs which have been selected to be PD-1, CD39, CD38, CD103, CD101, LAG3, TIM3 and/or TIGIT positive by culturing the first population of TILs in a cell culture medium comprising IL-2, OKT-3, and optionally comprising antigen presenting cells (APCs), to produce a second population of TILs, wherein the priming first expansion is performed for a first period of about 1 to 7, 8, 9, 10, or 11 days to obtain the second population of TILs, wherein the second population of TILs is greater in number than the first population of TILs;
(b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs; and
(c) harvesting the therapeutic population of TILs obtained from step (b).

8. The method of claim 7, wherein in step (a) the cell culture medium further comprises antigen-presenting cells (APCs), and wherein the number of APCs in the culture medium in step (b) is greater than the number of APCs in the culture medium in step (a).

9. The method of claim 7, wherein in step (a) the cell culture medium further comprises antigen-presenting cells (APCs), and wherein the number of APCs in the culture medium in step (b) is the equal to the number of APCs in the culture medium in step (a).

10. The method of claim 6 or 7, wherein said PD-1, CD39, CD38, CD103, CD101, LAG3, TIM3 and/or TIGIT positive TILs are PD-1high, CD39high, CD38lo, CD103high, CD101lo, LAG3high, TIM3high and/or TIGIThigh TILs.

11. The method of claim 1 or 2, wherein the selection of step (b), or the method of claim 6 or 7, wherein the selection of step (a), comprises a selection method selected from the group consisting of flow cytometry (including for example FACS), antibody-based bead selection, and antibody-based magnetic bead selection.

12. The method of claim 1 or 2, wherein the selection of step (b) or the method of claim 6 or 7, wherein the selection of step (a), comprises flow cytometry (including for example FACS).

13. The method of claim 1 or 2 or 12, wherein the selection of step (b) or the method of claim 6 or 7, wherein the selection of step (a), comprises the steps of (i) exposing the first population of TILs to an excess of a monoclonal anti-PD-1 IgG4 antibody that binds to PD-1 through an N-terminal loop outside the IgV domain of PD-1, (ii) adding an excess of an anti-IgG4 antibody conjugated to a fluorophore, and (iii) performing a flow-based cell sort based on the fluorophore to obtain a PD-1 enriched TIL population.

14. The method of claim 1 or 2 or 6 or 7, wherein the selection of PD-1, CD39, CD38, CD103, CD101, LAG3, TIM3 and/or TIGIT positive TILs occurs until there are at least 1×10⁶ PD-1, CD39, CD38, CD103, CD101, LAG3, TIM3 and/or TIGIT positive TILs.

15. The method of claim 1 or 2 or 6 or 7, wherein the cell culture medium for culturing the first population of TILs comprises 2-mercaptoethanol.

16. The method of claim 1 or 2 or 6 or 7, wherein the cell culture medium for culturing the second population of TILs comprises 2-mercaptoethanol.

17. The method of claim 1 or 2 or 6 or 7, wherein the cell culture medium for culturing the first population of TILs and the second population of TILs comprises 2-mercaptoethanol.

18. The method of claim 1 or 2 or 6 or 7, wherein the PD-1, CD39, CD38, CD103, CD101, LAG3, TIM3 and/or TIGIT positive TILs are selected using an anti-PD-1, anti-CD39, anti-CD38, anti-CD103, anti-CD101, anti-LAG3, anti-TIM3 and/or anti-TIGIT antibody conjugated bead, respectively.

19. The method of claim 1 or 2 or 6 or 7, wherein the PD-1, CD39, CD38, CD103, CD101, LAG3, TIM3 and/or TIGIT positive TILs are selected using an anti-PD-1, anti-CD39, anti-CD38, anti-CD103, anti-CD101, anti-LAG3, anti-TIM3 and/or anti-TIGIT antibody conjugated magnetic bead, respectively.

20. The method of claim 18 or 19, wherein the PD-1, CD39, CD38, CD103, CD101, LAG3, TIM3 and/or TIGIT positive TILs bind to an anti-PD-1, anti-CD39, anti-CD38, anti-CD103, anti-CD101, anti-LAG3, anti-TIM3 and/or anti-TIGIT antibody conjugated beads, respectively, and the PD-1, CD39, CD38, CD103, CD101, LAG3, TIM3 and/or TIGIT negative TILs do not bind to an anti-PD-1, anti-CD39, anti-CD38, anti-CD103, anti-CD101, anti-LAG3, anti-TIM3 and/or anti-TIGIT antibody conjugated bead, respectively.

21. The method of claim 13, wherein the monoclonal anti-PD-1 IgG4 antibody is nivolumab or variants, fragments, or conjugates thereof.

22. The method of claim 21, wherein the anti-IgG4 antibody is clone anti-human IgG4, Clone HP6023.

23. The method of claim 1 or 2 or 6 or 7, wherein the number of APCs in the rapid second expansion to the number of APCs in the priming first expansion is a ratio selected from a range of from about 1.5:1 to about 20:1.

24. The method of claim 23, wherein the ratio is selected from a range of from about 1.5:1 to about 10:1.

25. The method of claim 23, wherein the ratio is selected from a range of from about 2:1 to about 5:1.

26. The method of claim 23, wherein the ratio is selected from a range of from about 2:1 to about 3:1.

27. The method of claim 23, wherein the ratio is about 2:1.

28. The method of claim 1 or 2 or 6 or 7, wherein the number of APCs in the priming first expansion is selected from the range of about 1×10⁸ APCs to about 3.5×10⁸ APCs, and wherein the number of APCs in the rapid second expansion is selected from the range of about 3.5×10⁸ APCs to about 1×10⁹ APCs.

29. The method of claim 1 or 2 or 6 or 7, wherein the number of APCs in the priming first expansion is selected from the range of about 1.5×10⁸ APCs to about 3×10⁸ APCs, and wherein the number of APCs in the rapid second expansion is selected from the range of about 4×10⁸ APCs to about 7.5×10⁸ APCs.

30. The method of claim 1 or 2 or 6 or 7, wherein the number of APCs in the priming first expansion is selected from the range of about 2×10⁸ APCs to about 2.5×10⁸ APCs, and wherein the number of APCs in the rapid second expansion is selected from the range of about 4.5×10⁸ APCs to about 5.5×10⁸ APCs.

31. The method of claim 1 or 2 or 6 or 7, wherein about 2.5×10⁸ APCs are added to the priming first expansion and 5×10⁸ APCs are added to the rapid second expansion.

32. The method of any of claims 1-31, wherein the ratio of the number of TILs in the second population of TILs to the number of TILs in the first population of TILs is about 1.5:1 to about 100:1.

33. The method of any of claims 1-31, wherein the ratio of the number of TILs in the second population of TILs to the number of TILs in the first population of TILs is about 50:1.

34. The method of any of claims 1-31, wherein the ratio of the number of TILs in the second population of TILs to the number of TILs in the first population of TILs is about 25:1.

35. The method of any of claims 1-31, wherein the ratio of the number of TILs in the second population of TILs to the number of TILs in the first population of TILs is about 20:1.

36. The method of any of claims 1-31, wherein the ratio of the number of TILs in the second population of TILs to the number of TILs in the first population of TILs is about 10:1.

37. The method of any of claim 31, wherein the second population of TILs is at least 50-fold greater in number than the first population of TILs.

38. The method of any of claims 2-37, wherein the method comprises performing, after the step of harvesting the therapeutic population of TILs, the additional step of:
transferring the harvested therapeutic population of TILs to an infusion bag.

39. The method of any of claims 1-38, wherein the priming first expansion is performed in a plurality of separate containers, in each of which separate containers the second population of TILs is obtained from the first population of TILs in the step of the priming first expansion, and the third population of TILs is obtained from the second population of TILs in the step of the rapid second expansion, and wherein the therapeutic population of TILs obtained from the third population of TILs is collected from each of the plurality of containers and combined to yield the harvested TIL population.

40. The method of claim 39, wherein the plurality of separate containers comprises at least two separate containers.

41. The method of claim 39, wherein the plurality of separate containers comprises from two to twenty separate containers.

42. The method of claim 39, wherein the plurality of separate containers comprises from two to ten separate containers.

43. The method of claim 39, wherein the plurality of separate containers comprises from two to five separate containers.

44. The method of any of claims 39-43, wherein each of the separate containers comprises a first gas-permeable surface area.

45. The method of any of claims 1-38, wherein the priming first expansion step is performed in a single container.

46. The method of claim 45, wherein the single container comprises a first gas-permeable surface area.

47. The method of claim 45 or 46, wherein in the step of the priming first expansion the cell culture medium comprises antigen-presenting cells (APCs) and the APCs are layered onto the first gas-permeable surface area at an average thickness of about one cell layer to about three cell layers.

48. The method of claim 47, wherein in the step of the priming first expansion the APCs are layered onto the first gas-permeable surface area at an average thickness of about 1.5 cell layers to about 2.5 cell layers.

49. The method of claim 48, wherein in the step of the priming first expansion the APCs are layered onto the first gas-permeable surface area at an average thickness of about 2 cell layers.

50. The method of any of claims 47-49, wherein in the step of the rapid second expansion the APCs are layered onto the first gas-permeable surface area at a thickness of about 3 cell layers to about 5 cell layers.

51. The method of claim 50, wherein in the step of the rapid second expansion the APCs are layered onto the first gas-permeable surface area at a thickness of about 3.5 cell layers to about 4.5 cell layers.

52. The method of claim 51, wherein in the step of the rapid second expansion the APCs are layered onto the first gas-permeable surface area at a thickness of about 4 cell layers.

53. The method of any of claims 2-46, wherein in the step of the priming first expansion, the priming first expansion is performed in a first container comprising a first gas-permeable surface area, and in the step of the rapid second expansion, the rapid second expansion is performed in a second container comprising a second gas-permeable surface area.

54. The method of claim 53, wherein the second container is larger than the first container.

55. The method of claim 53 or 54, wherein in the step of the priming first expansion the cell culture medium comprises antigen-presenting cells (APCs) and the APCs are layered onto the first gas-permeable surface area at an average thickness of about one cell layer to about three cell layers.

56. The method of claim 55, wherein in the step of the priming first expansion the APCs are layered onto the first gas-permeable surface area at an average thickness of about 1.5 cell layers to about 2.5 cell layers.

57. The method of claim 55, wherein in the step of the priming first expansion the APCs are layered onto the first gas-permeable surface area at an average thickness of about 2 cell layers.

58. The method of any of claims 53-57, wherein in the step of the rapid second expansion the APCs are layered onto the second gas-permeable surface area at an average thickness of about 3 cell layers to about 5 cell layers.

59. The method of claim 58, wherein in the step of the rapid second expansion the APCs are layered onto the second gas-permeable surface area at an average thickness of about 3.5 cell layers to about 4.5 cell layers.

60. The method of claim 58, wherein in the step of the rapid second expansion the APCs are layered onto the second gas-permeable surface area at an average thickness of about 4 cell layers.

61. The method of any of claim 1-52, wherein for each container in which the priming first expansion is performed on a first population of TILs the rapid second expansion is performed in the same container on the second population of TILs produced from such first population of TILs.

62. The method of claim 61, wherein each container comprises a first gas-permeable surface area.

63. The method of claim 62, wherein in the step of the priming first expansion the cell culture medium comprises antigen-presenting cells (APCs) and the APCs are layered onto the first gas-permeable surface area at an average thickness of from about one cell layer to about three cell layers.

64. The method of claim 63, wherein in the step of the priming first expansion the APCs are layered onto the first gas-permeable surface area at an average thickness of from about 1.5 cell layers to about 2.5 cell layers.

65. The method of claim 64, wherein in the step of the priming first expansion the APCs are layered onto the first gas-permeable surface area at an average thickness of about 2 cell layers.

66. The method of any of claims 62-65, wherein in the step of the rapid second expansion the APCs are layered onto the first gas-permeable surface area at an average thickness of about 3 cell layers to about 5 cell layers.

67. The method of claim 66, wherein in the step of the rapid second expansion the APCs are layered onto the first gas-permeable surface area at an average thickness of about 3.5 cell layers to about 4.5 cell layers.

68. The method of claim 67, wherein in the step of the rapid second expansion the APCs are layered onto the first gas-permeable surface area at an average thickness of about 4 cell layers.

69. The method of any of claims 1-68, wherein for each container in which the priming first expansion is performed on a first population of TILs in the step of the priming first expansion the container comprises a first gas-permeable surface area, the cell culture medium comprises antigen-presenting cells (APCs), and the APCs are layered onto the first gas-permeable surface area, and wherein the ratio of the average number of layers of APCs layered in the step of the priming first expansion to the average number of layers of APCs layered in the step of the rapid second expansion is selected from the range of about 1:1.1 to about 1:10.

70. The method of claim 69, wherein the ratio of the average number of layers of APCs layered in the step of the priming first expansion to the average number of layers of APCs layered in the step of the rapid second expansion is selected from the range of about 1:1.2 to about 1:8.

71. The method of claim 69, wherein the ratio of the average number of layers of APCs layered in the step of the priming first expansion to the average number of layers of APCs layered in the step of the raid second expansion is selected from the range of about 1:1.3 to about 1:7.

72. The method of claim 69, wherein the ratio of the average number of layers of APCs layered in the step of the priming first expansion to the average number of layers of APCs layered in the step of the rapid second expansion is selected from the range of about 1:1.4 to about 1:6.

73. The method of claim 69, wherein the ratio of the average number of layers of APCs layered in the step of the priming first expansion to the average number of layers of APCs layered in the step of the rapid second expansion is selected from the range of about 1:1.5 to about 1:5.

74. The method of claim 69, wherein the ratio of the average number of layers of APCs layered in the step of the priming first expansion to the average number of layers of APCs layered in the step of the rapid second expansion is selected from the range of about 1:1.6 to about 1:4.

75. The method of claim 69, wherein the ratio of the average number of layers of APCs layered in the step of the priming first expansion to the average number of layers of APCs layered in the step of the rapid second expansion is selected from the range of about 1:1.7 to about 1:3.5.

76. The method of claim 69, wherein the ratio of the average number of layers of APCs layered in the step of the priming first expansion to the average number of layers of APCs layered in the step of the rapid second expansion is selected from the range of about 1:1.8 to about 1:3.

77. The method of claim 69, wherein the ratio of the average number of layers of APCs layered in the step of the priming first expansion to the average number of layers of APCs layered in the step of the rapid second expansion is selected from the range of about 1:1.9 to about 1:2.5.

78. The method of claim 69, wherein the ratio of the average number of layers of APCs layered in the step of the priming first expansion to the average number of layers of APCs layered in the step of the rapid second expansion is about 1:2.

79. The method of any of the preceding claims, wherein after 2 to 3 days in the step of the rapid second expansion, the cell culture medium is supplemented with additional IL-2.

80. The method according to any of the preceding claims, further comprising cryopreserving the harvested TIL population in the step of harvesting the therapeutic population of TILs using a cryopreservation process.

81. The method according to any of the preceding claims, further comprising the step of cryopreserving the infusion bag.

82. The method according to claim 80 or 81, wherein the cryopreservation process is performed using a 1:1 ratio of harvested TIL population to cryopreservation media.

83. The method according to any of the preceding claims, wherein the antigen-presenting cells are peripheral blood mononuclear cells (PBMCs).

84. The method according to claim 83, wherein the PBMCs are irradiated and allogeneic.

85. The method according to any of the preceding claims, wherein in the step of the priming first expansion the cell culture medium comprises peripheral blood mononuclear cells (PBMCs), and wherein the total number of PBMCs in the cell culture medium in the step of the priming first expansion is 2.5×10⁸.

86. The method according to any of preceding claims, wherein in the step of the rapid second expansion the antigen-presenting cells (APCs) in the cell culture medium are peripheral blood mononuclear cells (PBMCs), and wherein the total number of PBMCs added to the cell culture medium in the step of the rapid second expansion is 5×10⁸.

87. The method according to any of the preceding claims, wherein the antigen-presenting cells are artificial antigen-presenting cells.

88. The method according to any of the preceding claims, wherein the harvesting in the step of harvesting the therapeutic population of TILs is performed using a membrane-based cell processing system.

89. The method according to any of the preceding claims, wherein the harvesting in step (d) is performed using a LOVO cell processing system.

90. The method according to any of the preceding claims, wherein the multiple fragments comprise about 60 fragments per container in the step of the priming first expansion, wherein each fragment has a volume of about 27 mm³.

91. The method according to any of the preceding claims, wherein the multiple fragments comprise about 30 to about 60 fragments with a total volume of about 1300 mm³ to about 1500 mm³.

92. The method according to claim 91, wherein the multiple fragments comprise about 50 fragments with a total volume of about 1350 mm³.

93. The method according to any of the preceding claims, wherein the multiple fragments comprise about 50 fragments with a total mass of about 1 gram to about 1.5 grams.

94. The method according to any of the preceding claims, wherein the cell culture medium is provided in a container selected from the group consisting of a G-container and a Xuri cellbag.

95. The method of claim to any of the preceding claims, wherein after 2 to 3 days in step (d), the cell culture medium is supplemented with additional IL-2.

96. The method according to claim any of the preceding claims, wherein the IL-2 concentration is about 10,000 IU/mL to about 5,000 IU/mL.

97. The method according to claim any of the preceding claims, wherein the IL-2 concentration is about 6,000 IU/mL.

98. The method according to any of the preceding claims, wherein the infusion bag in the step of transferring the harvested therapeutic population of TILs to an infusion bag is a HypoThermosol-containing infusion bag.

99. The method according to any of claims 80-82, wherein the cryopreservation media comprises dimethlysulfoxide (DMSO).

100. The method according to claim 99, wherein the cryopreservation media comprises 7% to 10% DMSO.

101. The method according to any of the preceding claims, wherein the first period in the step of the priming first expansion and the second period in the step of the rapid second expansion are each individually performed within a period of 5 days, 6 days, 7 days, 8 days, 9 days, 10 days, or 11 days.

102. The method according to any of claims 1-101, wherein the first period in the step of the priming first expansion is performed within a period of 5 days, 6 days, or 7 days.

103. The method according to any of claims 1-101, wherein the first period in the step of the priming first expansion is performed within a period of 8 days, 9 days, 10 days, or 11 days.

104. The method according to any of claims 1-101, wherein the second period in the step of the rapid second expansion is performed within a period of 7 days, 8 days, or 9 days.

105. The method according to any of claims 1-101, wherein the second period in the step of the rapid second expansion is performed within a period of 10 days or 11 days.

106. The method according to any of claims 1-101, wherein the first period in the step of the priming first expansion and the second period in the step of the rapid second expansion are each individually performed within a period of 7 days.

107. The method according to any of claims 1-101, wherein the first period in the step of the priming first expansion and the second period in the step of the rapid second expansion are each individually performed within a period of 11 days.

108. The method according to any of claims 1-101, wherein steps of the priming first expansion through the harvesting of the therapeutic population of TILs are performed within a period of about 14 days to about 16 days.

109. The method according to any of claims 1-101, wherein steps of the priming first expansion through the harvesting of the therapeutic population of TILs are performed within a period of about 15 days to about 16 days.

110. The method according to any of claims 1-101, wherein steps of the priming first expansion through the harvesting of the therapeutic population of TILs are performed within a period of about 14 days.

111. The method according to any of claims 1-101, wherein steps of the priming first expansion through the harvesting of the therapeutic population of TILs are performed within a period of about 15 days.

112. The method according to any of claims 1-101, wherein steps the priming first expansion through the harvesting of the therapeutic population of TILs are performed within a period of about 16 days.

113. The method according to any of claims 1-101, further comprising the step of cryopreserving the harvested therapeutic population of TILs using a cryopreservation process, wherein steps of the priming first expansion through the harvesting of the therapeutic population of TILs and cryopreservation are performed in 16 days or less.

114. The method according to any one of claims 1 to 113, wherein the therapeutic population of TILs harvested in the step of harvesting of the therapeutic population of TILs comprises sufficient TILs for a therapeutically effective dosage of the TILs.

115. The method according to claim 114, wherein the number of TILs sufficient for a therapeutically effective dosage is from about 2.3×10¹⁰ to about 13.7×10¹⁰.

116. The method according to any one of claims 1 to 115, wherein the third population of TILs in the step of the rapid second expansion provides for increased efficacy, increased interferon-gamma production, and/or increased polyclonality.

117. The method according to any one of claims 1 to 115, wherein the third population of TILs in the step of the rapid second expansion provides for at least a one-fold to five-fold or more interferon-gamma production as compared to TILs prepared by a process longer than 16 days.

118. The method according to any one of claims 1 to 115, wherein the effector T cells and/or central memory T cells obtained from the third population of TILs in the step of the rapid second expansion exhibit increased CD8 and CD28 expression relative to effector T cells and/or central memory T cells obtained from the second population of TILs in the step of the priming first expansion.

119. The method according to any one of claims 1 to 118, wherein the therapeutic population of TILs from the step of the harvesting of the therapeutic population of TILs are infused into a patient.

120. The method according to claim 1 or 38, further comprising the step of cryopreserving the infusion bag comprising the harvested TIL population using a cryopreservation process.

121. The method according to claim 120, wherein the cryopreservation process is performed using a 1:1 ratio of harvested TIL population to cryopreservation media.

122. The method according to any of the preceding claims, wherein the antigen-presenting cells are peripheral blood mononuclear cells (PBMCs).

123. The method according to claim 122, wherein the PBMCs are irradiated and allogeneic.

124. The method according to any of claims 1-121, wherein the antigen-presenting cells are artificial antigen-presenting cells.

125. The method according to any of the preceding claims, wherein the harvesting step is performed using a membrane-based cell processing system.

126. The method according to any of the preceding claims, wherein the harvesting step is performed using a LOVO cell processing system.

127. The method according to claim 1 or 2, wherein the multiple fragments comprise about 60 fragments, and wherein each fragment has a volume of about 27 mm³.

128. The method according to claim 1 or 2, wherein the multiple fragments comprise about 30 to about 60 fragments with a total volume of about 1300 mm³ to about 1500 mm³.

129. The method according to claim 128, wherein the multiple fragments comprise about 50 fragments with a total volume of about 1350 mm³.

130. The method according to claim 1 or 2, wherein the multiple fragments comprise about 50 fragments with a total mass of about 1 gram to about 1.5 grams.

131. The method according to claim 1 or 2 or 6 or 7, wherein the cell culture medium is provided in a container selected from the group consisting of a G-container and a Xuri cellbag.

132. The method according to claim any of the preceding claims, wherein the IL-2 concentration is about 10,000 IU/mL to about 5,000 IU/mL.

133. The method according to claim any of the preceding claims, wherein the IL-2 concentration is about 6,000 IU/mL.

134. The method according to claim 1 or 2 or 6 or 7, wherein the infusion bag in step (d) is a HypoThermosol-containing infusion bag.

135. The method according to claim 121, wherein the cryopreservation media comprises dimethlysulfoxide (DMSO).

136. The method according to claim 135, wherein the wherein the cryopreservation media comprises 7% to 10% DMSO.

137. The method according to claim 1 or 2 or 6 or 7, wherein the first period and the second period in step (c) are each individually performed within a period of 5 days, 6 days, or 7 days.

138. The method according to claim 1 or 2 or 6 or 7, wherein the first period is performed within a period of 5 days, 6 days, or 7 days.

139. The method according to claim 1 or 2 or 6 or 7, wherein the second period is performed within a period of 10 or 11 days.

140. The method according to claim 1 or 2 or 6 or 7, wherein the first period and the second period are each individually performed within a period of 7 days.

141. The method according to claim 1 or 2 or 6 or 7, wherein all steps are performed within a period of about 14 days to about 22 days.

142. The method according to claim 1 or 2 or 6 or 7, wherein all steps are performed within a period of about 14 days to about 21 days.

143. The method according to claim 1 or 2 or 6 or 7, wherein all steps are performed within a period of about 14 days to about 20 days.

144. The method according to claim 1 or 2 or 6 or 7, wherein all steps are performed within a period of about 14 days to about 19 days.

145. The method according to claim 1 or 2 or 6 or 7, wherein all steps are performed within a period of about 14 days to about 18 days.

146. The method according to claim 1 or 2 or 6 or 7, wherein all steps are performed within a period of about 14 days to about 17 days.

147. The method according to claim 1 or 2 or 6 or 7, wherein all steps are performed within a period of about 14 days to about 16 days.

148. The method according to claim 1 or 2 or 6 or 7, wherein all steps are performed within a period of about 15 days to about 16 days.

149. The method according to claim 1 or 2 or 6 or 7, wherein all steps are performed within a period of about 14 days.

150. The method according to claim 1 or 2 or 6 or 7, wherein all steps are performed within a period of about 15 days.

151. The method according to claim 1 or 2 or 6 or 7, wherein all steps are performed within a period of about 16 days.

152. The method according to claim 151, wherein all steps and cryopreservation are performed in 16 days or less.

153. The method according to any one of claims 1 to 152, wherein the therapeutic population of TILs harvested comprises sufficient TILs for a therapeutically effective dosage of the TILs.

154. The method according to claim 153, wherein the number of TILs sufficient for a therapeutically effective dosage is from about 2.3×10¹⁰ to about 13.7×10¹⁰.

155. The method according to any one of claims 1 to 154, the container in the priming first expansion step is larger than the container in the rapid second expansion step.

156. The method according to any one of claims 1 to 155, wherein the third population of TILs provides for increased efficacy, increased interferon-gamma production, and/or increased polyclonality.

157. The method according to any one of claims 1 to 156, wherein the third population of TILs provides for at least a one-fold to five-fold or more interferon-gamma production as compared to TILs prepared by a process longer than 16 days.

158. The method according to any one of claims 1 to 157, wherein the effector T cells and/or central memory T cells obtained from the third population of TILs exhibit increased CD8 and CD28 expression relative to effector T cells and/or central memory T cells obtained from the second population of cells.

159. The method according to any one of claims 1 to 158, wherein the harvested TILs are infused into a patient.

160. A method for treating a subject with cancer, the method comprising administering expanded tumor infiltrating lymphocytes (TILs) comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for about 1 to 7, 8, 9, 10, or 11 days to obtain the second population of TILs;
(d) performing a rapid second expansion by supplementing the cell culture medium of the second population of TILs with additional IL-2, OKT-3, and APCs, to produce a third population of TILs, wherein the number of APCs added to the rapid second expansion is at least twice the number of APCs added in step (b), wherein the rapid second expansion is performed for about 1 to 11 days to obtain the third population of TILs, wherein the third population of TILs is a therapeutic population of TILs, wherein the rapid second expansion is performed in a container comprising a second gas-permeable surface area;
(e) harvesting the therapeutic population of TILs obtained from step (c);
(f) transferring the harvested TIL population from step (d) to an infusion bag; and
(g) administering a therapeutically effective dosage of the TILs from step (e) to the subject.

161. The method according to claim 160, wherein the number of TILs sufficient for administering a therapeutically effective dosage in step (g) is from about 2.3×10¹⁰ to about 13.7×10¹⁰.

162. The method according to claim 160 or 161, wherein said PD-1, CD39, CD38, CD103, CD101, LAG3, TIM3 and/or TIGIT positive TILs are PD-1high, CD39high, CD38lo, CD103high/lo, CD101lo, LAG3high, TIM3high and/or TIGIThigh TILs.

163. The method according to any one of claims 160-162, wherein the selection of step (b) comprises the steps of (i) exposing the first population of TILs to an excess of a monoclonal anti-PD-1 IgG4 antibody that binds to PD-1 through an N-terminal loop outside the IgV domain of PD-1, (ii) adding an excess of an anti-IgG4 antibody conjugated to a fluorophore, and (iii) performing a flow-based cell sort based on the fluorophore to obtain a PD-1 enriched TIL population.

164. The method of claim 163, wherein the monoclonal anti-PD-1 IgG4 antibody is nivolumab or variants, fragments, or conjugates thereof.

165. The method of claim 164, wherein the anti-IgG4 antibody is clone anti-human IgG4, Clone HP6023.

166. The method according to claim 165, wherein the antigen presenting cells (APCs) are PBMCs.

167. The method according to any of claims 160 to 166, wherein prior to administering a therapeutically effective dosage of TIL cells in step (g), a non-myeloablative lymphodepletion regimen has been administered to the subject.

168. The method according to claim 167, where the non-myeloablative lymphodepletion regimen comprises the steps of administration of cyclophosphamide at a dose of 60 mg/m²/day for two days followed by administration of fludarabine at a dose of 25 mg/m²/day for five days.

169. The method according to any of claims 160 to 168, further comprising the step of treating the patient with a high-dose IL-2 regimen starting on the day after administration of the TIL cells to the subject in step (g).

170. The method according to claim 169, wherein the high-dose IL-2 regimen comprises 600,000 or 720,000 IU/kg administered as a 15-minute bolus intravenous infusion every eight hours until tolerance.

171. The method according to any one of claims 160 to 170, wherein the third population of TILs in step (c) provides for increased efficacy, increased interferon-gamma production, and/or increased polyclonality.

172. The method according to any one of claims 160 to 171, wherein the third population of TILs in step (d) provides for at least a one-fold to five-fold or more interferon-gamma production as compared to TILs prepared by a process longer than 16 days.

173. The method according to any one of claims 160 to 172, wherein the effector T cells and/or central memory T cells obtained from the third population of TILs exhibit increased CD8 and CD28 expression relative to effector T cells and/or central memory T cells obtained from the second population of TILs.

174. The method according to any of claims 160-172, wherein the cancer is selected from the group consisting of melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma.

175. The method according to any of claims 160-172, wherein the cancer is selected from the group consisting of melanoma, HNSCC, cervical cancers, NSCLC, glioblastoma (including GBM), and gastrointestinal cancer.

176. The method according to any claims 160-172, wherein the cancer is melanoma.

177. The method according to any of claims 160-172, wherein the cancer is HNSCC.

178. The method according to any claims 160-172, wherein the cancer is a cervical cancer.

179. The method according to any of claims 160-172, wherein the cancer is NSCLC.

180. The method according to any of claims 160-172, wherein the cancer is glioblastoma (including GBM).

181. The method according to any of claims 160-172, wherein the cancer is gastrointestinal cancer.

182. The method according to any of claims 160-172, wherein the cancer is a hypermutated cancer.

183. The method according to any of claims 160-172, wherein the cancer is a pediatric hypermutated cancer.

184. The method according to any of the preceding claims, wherein the priming first expansion is performed in a first container and the rapid second expansion is performed in a second container, and wherein each of the first and second containers is a GREX-10.

185. The method according to any of claims 1-184, wherein the priming first expansion is performed in a first closed container and the rapid second expansion is performed in a second closed container, and wherein each of the first and second closed containers comprises a GREX-100.

186. The method according to any of claims 1-184, wherein the priming first expansion is performed in a first closed container and the rapid second expansion is performed in a second closed container, and wherein the each of the first and second closed containers comprises a GREX-500.

187. The method according to any of claims 160-183, wherein the subject has been previously treated with an anti-PD-1 antibody.

188. The method according to any of claims 160-183, wherein the subject has not been previously treated with an anti-PD-1 antibody.

189. The method according to any of the preceding claims, wherein the priming first expansion step is performed on a first population of TILs selected or enriched for PD-1 positive TILs by contacting the first population of TILs with an anti-PD-1 antibody to form a first complex of the anti-PD-1 antibody and TIL cells in the first population of TILs, and then isolating the first complex to obtain the first population of TILs selected or enriched for PD-1 positive TILs.

190. The method of claim 189, wherein the anti-PD-1 antibody comprises an Fc region, wherein after the step of forming the first complexes and before the step of isolating the first complex the method further comprises the step of contacting the first complex with an anti-Fc antibody that binds to the Fc region of the anti-PD-1 antibody to form a second complex of the anti-Fc antibody and the first complex, and wherein the step of isolating the first complex is performed by isolating the second complex.

191. The method according to claim 189 or 190, wherein the anti-PD-1 antibody is selected from the group consisting of EH12.2H7, PD1.3.1, SYM021, M1H4, A1 7188B, nivolumab (BMS-936558, Bristol-Myers Squibb; Opdivo®), pembrolizumab (lambrolizumab, MK03475 or MK-3475, Merck; Keytruda®), H12.1, PD1.3.1, NAT 105, humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), humanized anti-PD-1 IgG4 antibody PDR001 (Novartis), and RMP1-14 (rat IgG)—BioXcell cat #BP0146.

192. The method according to claim 189 or 190, wherein the anti-PD-1 antibody is EH12.2H7.

193. The method according to claim 189 or 190, wherein the anti-PD-1 antibody binds to a different epitope than nivolumab or pembrolizumab.

194. The method according to claim 189 or 190, wherein the anti-PD-1 antibody binds to the same epitope as EH12.2H7 or nivolumab.

195. The method according to claim 189 or 190, wherein the anti-PD-1 antibody is nivolumab.

196. The method of any of claims 160-183, wherein the subject has been previously treated with a first anti-PD1 antibody, wherein the priming first expansion step is performed on a first population of TILs selected or enriched for PD-1 positive TILs by contacting the first population of TILs with a second anti-PD-1 antibody to form a first complex of the second anti-PD-1 antibody and TIL cells in the first population of TILs, and then isolating the first complex to obtain the first population of TILs selected or enriched for PD-1 positive TILs, and wherein the second anti-PD-1 antibody is not blocked from binding to the first population of TILs by the first anti-PD-1 antibody insolubilized on the first population of TILs.

197. The method of claim 160-183, wherein the subject has been previously treated with a first anti-PD1 antibody, wherein the priming first expansion step is performed on a first population of TILs selected or enriched for PD-1 positive TILs by contacting the first population of TILs with a second anti-PD-1 antibody to form a first complex of the second anti-PD-1 antibody and TIL cells in the first population of TILs, and then isolating the first complex to obtain the first population of TILs selected or enriched for PD-1 positive TILs, and wherein the second anti-PD-1 antibody is blocked from binding to the first population of TILs by the first anti-PD-1 antibody insolubilized on the first population of TILs.

198. The method of claim 196 or 197, wherein the first anti-PD-1 antibody and the second anti-PD-1 antibody comprise an Fc region, wherein after the step of forming the first complex and before the step of isolating the first complex the method further comprises the step of contacting the first complex with an anti-Fc antibody that binds to the Fc region of the first anti-PD-1 antibody and the Fc region of the second anti-PD-1 antibody to form a second complex of the anti-Fc antibody and the first complex, and wherein the step of isolating the first complex is performed by isolating the second complex.

199. The method of any of claims 160-183, wherein the subject has been previously treated with a first anti-PD1 antibody, wherein the priming first expansion step is performed on a first population of TILs selected or enriched for PD-1 positive TILs by (i) contacting the first population of TILs with a second anti-PD-1 antibody to form a first complex of the second anti-PD-1 antibody and the first population of TILs, wherein the second anti-PD-1 antibody is blocked from binding to the PD-1 positive TILs by the first anti-PD-1 antibody insolubilized on the first population of TILs, wherein the first anti-PD-1 antibody and the second anti-PD-1 antibody comprise an Fc region, (ii) contacting the first complex with an anti-Fc antibody that binds to the Fc region of the second anti-PD-1 antibody to form a second complex of the anti-Fc antibody and the first complex and contacting the first anti-PD-1 antibody insolubilized on the first population of TILs with the anti-Fc antibody to form a third complex of the anti-Fc antibody and the first anti-PD-1 antibody insolubilized on the first population of TILs, and (iii) isolating the second and third complexes to obtain the first population of TILs selected or enriched for PD-1 positive TILs.

200. A therapeutic population of tumor infiltrating lymphocytes (TILs) prepared from PD-1, LAG3, TIM3 and/or TIGIT positive cells selected from a digest of a tumor tissue sample obtained from a patient, wherein the therapeutic population of TILs provides for increased efficacy and/or increased interferon-gamma production.

201. The therapeutic population of TILs of claim 200 that provides for increased interferon-gamma production.

202. The therapeutic population of TILs of claim 200 or claim 201 that provides for increased efficacy.

203. The therapeutic population of TILs of any of claims 200-202, wherein the therapeutic population of TILs is capable of at least one-fold more interferon-gamma production as compared to TILs prepared by a process longer than 16 days.

204. The therapeutic population of TILs of any of claims 200-203, wherein the therapeutic population of TILs is capable of at least one-fold more interferon-gamma production as compared to TILs prepared by a process longer than 16-22 days.

205. The method according to any of the preceding claims, wherein the priming first expansion step is performed on a first population of TILs selected or enriched for PD-1, LAG3, TIM3 and/or TIGIT positive TILs with at least 11.27% to 74.4% PD-1 positive TILs.

206. The method according to any of the preceding claims, wherein the priming first expansion step is performed on a first population of TILs selected or enriched for PD-1 positive TILs by the steps of:
(i) exposing the first population of TILs and a population of PBMC to an excess of a monoclonal anti-PD-1 IgG4 antibody that binds to PD-1 through an N-terminal loop outside the IgV domain of PD-1,
(ii) adding an excess of an anti-IgG4 antibody conjugated to a fluorophore, and
(iii) obtaining the first population of TILs selected or enriched for PD-1 positive TILs based on the intensity of the fluorophore of the PD-1 positive TILs in the first population of TILs compared to the intensity in the population of PBMCs as performed by fluorescence-activated cell sorting (FACS).

207. The method of claim 206, wherein the intensity of the fluorophore in both the first population of TILs and the population of PBMCs is used to set up FACS gates for establishing low, medium, and high levels of intensity that correspond to PD-1 negative TILs, PD-1 intermediate TILs, and PD-1 positive TILs, respectively.

208. The method of claim 207, wherein the FACS gates are set-up after step (a).

209. The method according to any one of the preceding claims, wherein the PD-1, LAG3, TIM3 and/or TIGIT positive TILs are PD-1high, LAG3high, TIM3high and/or TIGIThigh TILs.

210. The method according to any one of the preceding claims, wherein at least 80% of the first population of TILs selected or enriched for PD-1 positive TILs are PD-1 positive TILs, at least 80% of the first population of TILs selected or enriched for LAG3 positive TILs are LAG3 positive TILs, at least 80% of the first population of TILs selected or enriched for TIM3 positive TILs are TIM3 positive TILs, and/or at least 80% of the first population of TILs selected or enriched for TIGIT positive TILs are TIGIT positive TILs.

211. A method for expanding tumor infiltrating lymphocytes (TILs) into a therapeutic population of TILs comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population, wherein at least a range of 10% to 80% of the first population of TILs are PD-1, CD39, CD38, CD103, CD101, LAG3, TIM3 and/or TIGIT positive TILs;
(c) performing a priming first expansion by culturing the PD-1, CD39, CD38, CD103, CD101, LAG3, TIM3 and/or TIGIT enriched TIL population in a cell culture medium comprising IL-2, OKT-3, and antigen presenting cells (APCs) to produce a second population of TILs, wherein the priming first expansion is performed in a container comprising a first gas-permeable surface area; wherein the priming first expansion is performed for first period of about 1 to 7, 8, 9, 10, or 11 days to obtain the second population of TILs, wherein the second population of TILs is greater in number than the first population of TILs;
(d) performing a rapid second expansion by supplementing the cell culture medium of the second population of TILs with additional IL-2, OKT-3, and APCs, to produce a third population of TILs, wherein the number of APCs added in the rapid second expansion is at least twice the number of APCs added in step (c), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(e) harvesting the therapeutic population of TILs obtained from step (d); and
(f) transferring the harvested TIL population from step (e) to an infusion bag.

212. The method according to claim 211, wherein the selection of step (b) comprises the steps of:
(i) exposing the first population of TILs and a population of PBMC to an excess of a monoclonal anti-PD-1 IgG4 antibody that binds to PD-1 through an N-terminal loop outside the IgV domain of PD-1,
(ii) adding an excess of an anti-IgG4 antibody conjugated to a fluorophore,
(iii) obtaining the PD-1 enriched TIL population based on the intensity of the fluorophore of the PD-1 positive TILs in the first population of TILs compared to the intensity in the population of PBMCs as performed by fluorescence-activated cell sorting (FACS).

213. The method according to claim 212, wherein the intensity of the fluorophore in both the first population and the population of PBMCs is used to set up FACS gates for establishing low, medium, and high levels of intensity that correspond to:
i) PD-1 negative TILs, PD-1 low TILs, PD-1 intermediate TILs, and PD-1 positive TILs, respectively;
ii) CD39 negative TILs, CD39 low TILs, CD39 intermediate TILs, and CD39 positive TILs, respectively;
iii) CD38 negative TILs, CD38 low TILs, CD38 intermediate TILs, and CD38 positive TILs, respectively;
iv) CD103 negative TILs, CD103 low TILs, CD103 intermediate TILs, and CD103 positive TILs, respectively;
v) CD101 negative TILs, CD101 low TILs, CD101 intermediate TILs, and CD101 positive TILs, respectively;
vi) LAG3 negative TILs, LAG3 low TILs, LAG3 intermediate TILs, and LAG3positive TILs, respectively;
vii) TIM3 negative TILs, TIM3 low TILs, TIM3 intermediate TILs, and TIM3 positive TILs, respectively; and/or
viii) TIGIT negative TILs, TIGIT low TILs, TIGIT intermediate TILs, and TIGIT positive TILs, respectively.

214. The method according to any one of claims 211-213, wherein the FACS gates are set-up after step (a).

215. The method according to any one of claims 211-214, wherein the PD-1, LAG3, TIM3 and/or TIGIT positive TILs are PD-1high, LAG3high, TIM3high and/or TIGIThigh TILs.

216. The method according to any one of claims 211-215, wherein at least 80% of the PD-1, LAG3, TIM3 and/or TIGIT enriched TIL population are PD-1, LAG3, TIM3 and/or TIGIT positive TILs.

217. The method according to any one of claims 211-216, wherein the third population of TILs comprises at least about 1×10⁸ TILs in the container.

218. The method according to any one of claims 211-217, wherein the third population of TILs comprises at least about 1×10⁹ TILs in the container.

219. The method according to any one of claims 211-218, wherein the number of PD-1, LAG3, TIM3 and/or TIGIT enriched TILs in the priming first expansion is from about 1×10⁴ to about 1×10⁶.

220. The method according to any one of claims 211-219, wherein the number of PD-1, LAG3, TIM3 and/or TIGIT enriched TILs in the priming first expansion is from about 5×10⁴ to about 1×10⁶.

221. The method according to any one of claims 211-220, wherein the number of PD-1, LAG3, TIM3 and/or TIGIT enriched TILs in the priming first expansion is from about 2×10⁵ to about 1×10⁶.

222. The method according to any one of claims 211-221, further comprising the step of cyropreserving the first population of TILs from the tumor resected from the subject before performing step (a).

223. A method for expanding tumor infiltrating lymphocytes (TILs) into a therapeutic population of TILs comprising:
(a) obtaining and/or receiving a first population of TILs from a tumor resected from a subject by processing a tumor sample obtained from the subject into multiple tumor fragments;
(b) selecting PD-1, CD39, CD38, CD103, CD101, LAG3, TIM3 and/or TIGIT positive TILs from the first population of TILs in (a) to obtain a PD-1, CD39, CD38, CD103, CD101, LAG3, TIM3 and/or TIGIT enriched TIL population;
(c) performing a priming first expansion by culturing the first population of TILs in a first TIL cell culture comprising a first cell culture medium, IL-2, and either:
i) a first culture supernatant obtained from a first culture of antigen-presenting feeder cells (APCs), wherein the first culture supernatant comprises OKT-3, or
ii) APCs and OKT-3,
wherein the priming first expansion is performed by culturing the first TIL cell culture in a first container comprising a first gas-permeable surface area for a first period of about 1 to 7, 8, 9, 10, or 11 days to obtain a second population of TILs, and wherein the second population of TILs is greater in number than the first population of TILs;
(d) performing a rapid second expansion by transferring the first TIL cell culture into a second container comprising a second gas-permeable surface area supplemented with a second cell culture medium, IL-2, and either:
i) a second culture supernatant obtained from a second culture of APCs, wherein the second culture supernatant comprises OKT-3, or
ii) APCs and OKT-3;
to form a second TIL cell culture, wherein the rapid second expansion is performed by culturing the second TIL cell culture for a second period of about 1 to 11 days to obtain a third population of TILs, and wherein the third population of TILs is a therapeutic population of TILs; wherein the first TIL cell culture does not comprise both the first culture supernatant and APCs; wherein the second TIL cell culture does not comprise both the second culture supernatant and supplemental APCs;
(e) harvesting the therapeutic population of TILs obtained from step (d); and
(f) transferring the harvested TIL population from step (e) to an infusion bag.

224. The method for expanding TILs according to claim 223, wherein in the priming first expansion of step (c) the first TIL cell culture comprises the first culture supernatant, and wherein in the rapid second expansion of step (d) the first TIL cell culture is supplemented with OKT-3 and APCs to form the second TIL cell culture.

225. The method for expanding TILs according to claim 223 or 224, wherein in the priming first expansion of step (c) the first TIL cell culture comprises OKT-3 and APCs, and wherein in the rapid second expansion of step (d) the first TIL cell culture is supplemented with the second culture supernatant to form the second TIL cell culture.

226. The method for expanding TILs according to claims 223-225, wherein in the priming first expansion of step (c) the first TIL cell culture comprises the first culture supernatant, and wherein in the rapid second expansion of step (d) the first TIL cell culture is supplemented with the second culture supernatant to form the second TIL cell culture.

227. The method for expanding TILs according to claims 223-226, wherein obtaining the first culture supernatant for use in step (c) comprises:
1) providing an APC cell culture medium comprising IL-2 and OKT-3;
2) culturing at least about 5×10⁸ APCs in the APC cell culture medium from 1) for about 3-4 days to generate the first culture supernatant; and
3) collecting the first culture supernatant from the cell culture in 2).

228. The method for expanding TILs according to claims 223-227, wherein obtaining the second culture supernatant for use in step (d) comprises:
1) providing an APC cell culture medium comprising IL-2 and OKT-3;
2) culturing at least about 1×10⁷ APCs in the APC cell culture medium from 1) for about 3-4 days to generate the second culture supernatant; and
3) collecting the second culture supernatant from the cell culture in 2).

229. The method of claims 223-228, wherein the rapid second expansion of step (d) further comprises the step of:
i) supplementing the second TIL cell culture with additional IL-2 about 3 or 4 days after the initiation of the second period in step (d).

230. The method of claims 223-229, wherein the APCs are exogenous to the subject.

231. The method of claims 223-230, wherein the APCs are peripheral blood mononuclear cells (PBMCs).

232. The method of claims 223-231, wherein the rapid second expansion of step (d) further comprises the steps of:
i) on or about 3 or 4 days after the initiation of the second period, transferring the second TIL cell culture from the second container into a plurality of third containers to form a subculture of the second TIL cell culture in each of the plurality of third containers; and
ii) culturing the subculture of the second TIL cell culture in each of the plurality of third containers for the remainder of the second period.

233. The method of claim 232, wherein in step i) equal volumes of the second TIL cell culture are transferred into the plurality of third containers.

234. The method of claim 232 or 233, wherein each of the third containers is equal in size to the second container.

235. The method of claim 232 or 233, wherein each of the third containers is larger than the second container.

236. The method of claims 232-235, wherein the third containers are equal in size.

237. The method of claim 236, wherein the third containers are larger than the second container.

238. The method of claim 232 or 233, wherein the third containers are smaller than the second container.

239. The method of claims 223-238, wherein the second container is a G-Rex 100M flask.

240. The method of claim 239, wherein the second container is a G-Rex 100M flask and each of the plurality of third containers is a G-Rex 100M flask.

241. The method of claim 240, wherein the plurality of third containers is selected from the group consisting of: 2, 3, 4, 5, 6, 7, 8, 9, 10, 11, 12, 13, 14, 15, 16, 17, 18, 19 and 20 second containers.

242. The method of claim 240, wherein the plurality of second containers is 2 third containers.

243. The method of claim 240, wherein before step ii) the method further comprises supplementing each subculture of the second TIL cell culture with additional IL-2.

244. The method of claim 240, wherein before step ii) the method further comprises supplementing each subculture of the second TIL cell culture with a second cell culture medium and IL-2.

245. The method of any of claims 223-244, wherein the first cell culture medium and the second cell culture medium are the same.

246. The method of any of claims 223-244, wherein the first cell culture medium and the second cell culture medium are different.

247. The method of any of claims 223-244, wherein the first cell culture medium is DM1 and the second cell culture medium is DM2.

248. The method according to any of the preceding claims, wherein the TILs are selected as PD-1 positive (PD-1+), LAG3 positive (LAG3+ positive), CD38 positive (CD38+), and CD101 positive (CD101+).

249. The method according to any of the preceding claims, wherein the TILs are selected as PD-1high, LAG3high, CD38lo, and CD101lo.

250. The method according to any of the preceding claims, wherein the TILs are selected as PD-1 positive (PD-1+), LAG3 positive (LAG3+ positive), and CD38 positive (CD38+).

251. The method according to any of the preceding claims, wherein the TILs are selected as PD-Thigh, LAG3high, and CD38lo.

252. The method according to any of the preceding claims, wherein the TILs are selected as PD-1 positive (PD-1+), LAG3 positive (LAG3+ positive), and CD101 positive (CD101+).

253. The method according to any of the preceding claims, wherein the TILs are selected as PD-1high, LAG-3high, and CD101lo.

254. The method according to any of the preceding claims, wherein the TILs are selected as PD-1 positive (PD-1+) and CD38 positive (CD38+).

255. The method according to any of the preceding claims, wherein the TILs are selected as PD-1hi and CD38lo.

256. The method according to any of the preceding claims, wherein the TILs are selected as PD-1 positive (PD-1+) and CD101 positive (CD101+).

257. The method according to any of the preceding claims, wherein the TILs are selected as PD-1high and CD101lo.

258. The method according to any of the preceding claims, wherein the selection comprises a selection method selected from the group consisting of flow cytometry (including for example FACS), antibody-based bead selection, and antibody-based magnetic bead selection.

259. The method of claim 258, wherein the selection method comprises flow cytometry (including for example FACS).

260. The method of claim 258, wherein the selection methods comprises an antibody-based bead selection.

261. The method of claim 258, wherein the selection comprises an antibody-based magnetic bead selection.

262. The method according to any of the preceding claims, wherein the selection comprises a two step selection, comprising:
i) a first selection step comprising a method that selects for PD-1+, LAG3+, TIM3+ and/or TIGIT+, and
ii) a second selection step comprising a method that selects for CD38+ and/or CD101+.

263. The method of claim 262, where the first selection step comprises a method that selects for PD-1high, LAG3high, TIM3high and/or TIGIThigh.

264. The method of claim 262, where the first selection step comprises a method that selects for PD-1high or LAG3high.

265. The method of claim 263 or 264, where the second selection step comprises a method that selects for CD3810 and/or CD101lo.

266. The method of claims 262-265, wherein the first selection step comprises flow cytometry (including for example FACS) and wherein the second selection step comprises flow cytometry (including for example FACS).

267. The method of claims 262-265, wherein the first selection step comprises an antibody-based bead selection and wherein the second selection step comprises flow cytometry (including for example FACS).

268. The method of claims 262-265, wherein the first selection step comprises an antibody-based magnetic bead selection and wherein the second selection step comprises flow cytometry (including for example FACS).

269. The method of claims 262-265, wherein the first selection step comprises an antibody-based bead selection or antibody-based magnetic bead selection and the second selection step comprises an antibody-based bead selection or antibody-based magnetic bead selection.

270. The method of claim 269, wherein the beads used in the antibody-based bead selection for PD-1+, LAG3+, TIM3+ and/or TIGIT+ TILs are anti-PD-1, anti-LAG3, anti-TIM3 and/or anti-TIGIT antibody conjugated beads, respectively.

271. The method of claim 269 or 270, wherein the beads used in the antibody-based bead selection for CD38+ or CD101+ TILs are anti-CD38 or anti-CD101 antibody conjugated beads, respectively.

272. The method of claims 269-271, wherein the beads used in the antibody-based magnetic bead selection for PD-1+, LAG3+, TIM3+ and/or TIGIT+ TILs are anti-PD-1, anti-LAG3, anti-TIM3 and/or anti-TIGIT antibody conjugated magnetic beads, respectively.

273. The method of claims 269-272, wherein the beads used in the antibody-based magnetic bead selection for CD38+ or CD101+ TILs are anti-CD38 or anti-CD101 antibody conjugated magnetic beads, respectively.

274. The method of claims 269-273, wherein the PD-1+, LAG3+, TIM3+ and/or TIGIT+ TILs bind to an anti-PD-1, anti-LAG3, anti-TIM3 and/or anti-TIGIT antibody conjugated beads, respectively, and the PD-1, LAG3, TIM3 and/or TIGIT negative TILs do not bind to an anti-PD-1, anti-LAG3, anti-TIM3 and/or anti-TIGIT antibody conjugated beads, respectively.

275. The method of any of the preceding claims, wherein the priming first expansion step is performed on a first population of TILs selected or enriched from a digest of a tumor sample obtained from a patient or subject.

276. The method of claim 275, wherein the digest is performed with a mixture of enzymes.

277. The method of claim 276, wherein the mixture of enzymes comprises a neutral protease, a collagenase, and a DNase.


SEQUENCE LISTING (569 sequences)

SEQ ID NO: 1 - protein, 450 aa, Artificial Sequence
  Synthetic Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Synthetic Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, Artificial Sequence
  Synthetic recombinant human IL-2 (rhIL-2)
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, Artificial Sequence
  Synthetic Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 130 aa, Artificial Sequence
  Synthetic recombinant human IL-4 (rhIL-4)
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 153 aa, Artificial Sequence
  Synthetic recombinant human IL-7 (rhIL-7)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 115 aa, Artificial Sequence
  Synthetic recombinant human IL-15 (rhIL-15)
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 132 aa, Artificial Sequence
  Synthetic recombinant human IL-21 (rhIL-21)
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 9 - protein, 255 aa, Artificial Sequence
  Synthetic human 4-1BB, Tumor necrosis factor receptor superfamily, member 9 (Homo sapiens)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 10 - protein, 256 aa, Artificial Sequence
  Synthetic murine 4-1BB, Tumor necrosis factor receptor superfamily, member 9 (Mus musculus)
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 11 - protein, 441 aa, Artificial Sequence
  Synthetic heavy chain for utomilumab
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 12 - protein, 214 aa, Artificial Sequence
  Synthetic light chain for utomilumab
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 116 aa, Artificial Sequence
  Synthetic heavy chain variable region for utomilumab
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 14 - protein, 108 aa, Artificial Sequence
  Synthetic light chain variable region for utomilumab
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 15 - protein, 6 aa, Artificial Sequence
  Synthetic heavy chain CDR1 for utomilumab
STYWIS

SEQ ID NO: 16 - protein, 17 aa, Artificial Sequence
  Synthetic heavy chain CDR2 for utomilumab
KIYPGDSYTNYSPSFQG

SEQ ID NO: 17 - protein, 8 aa, Artificial Sequence
  Synthetic heavy chain CDR3 for utomilumab
RGYGIFDY

SEQ ID NO: 18 - protein, 11 aa, Artificial Sequence
  Synthetic light chain CDR1 for utomilumab
SGDNIGDQYAH

SEQ ID NO: 19 - protein, 7 aa, Artificial Sequence
  Synthetic light chain CDR2 for utomilumab
QDKNRPS

SEQ ID NO: 20 - protein, 11 aa, Artificial Sequence
  Synthetic light chain CDR3 for utomilumab
ATYTGFGSLAV

SEQ ID NO: 21 - protein, 448 aa, Artificial Sequence
  Synthetic heavy chain for urelumab
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 216 aa, Artificial Sequence
  Synthetic light chain for urelumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 120 aa, Artificial Sequence
  Synthetic variable heavy chain for urelumab
MKHLWFFLLLVAAPRWVLSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGP

SEQ ID NO: 24 - protein, 110 aa, Artificial Sequence
  Synthetic variable light chain for urelumab
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQ

SEQ ID NO: 25 - protein, 5 aa, Artificial Sequence
  Synthetic heavy chain CDR1 for urelumab
GYYWS

SEQ ID NO: 26 - protein, 16 aa, Artificial Sequence
  Synthetic heavy chain CDR2 for urelumab
EINHGGYVTYNPSLES

SEQ ID NO: 27 - protein, 13 aa, Artificial Sequence
  Synthetic heavy chain CDR3 for urelumab
DYGPGNYDWYFDL

SEQ ID NO: 28 - protein, 11 aa, Artificial Sequence
  Synthetic light chain CDR1 for urelumab
RASQSVSSYLA

SEQ ID NO: 29 - protein, 7 aa, Artificial Sequence
  Synthetic light chain CDR2 for urelumab
DASNRAT

SEQ ID NO: 30 - protein, 11 aa, Artificial Sequence
  Synthetic light chain CDR3 for urelumab
QQRSDWPPALT

SEQ ID NO: 31 - protein, 230 aa, Artificial Sequence
  Synthetic Fc domain
KSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 22 aa, Artificial Sequence
  Synthetic linker
GGPGSSKSCDKTHTCPPCPAPE

SEQ ID NO: 33 - protein, 22 aa, Artificial Sequence
  Synthetic linker
GGSGSSKSCDKTHTCPPCPAPE

SEQ ID NO: 34 - protein, 27 aa, Artificial Sequence
  Synthetic linker
GGPGSSSSSSSKSCDKTHTCPPCPAPE

SEQ ID NO: 35 - protein, 27 aa, Artificial Sequence
  Synthetic linker
GGSGSSSSSSSKSCDKTHTCPPCPAPE

SEQ ID NO: 36 - protein, 29 aa, Artificial Sequence
  Synthetic linker
GGPGSSSSSSSSSKSCDKTHTCPPCPAPE

SEQ ID NO: 37 - protein, 29 aa, Artificial Sequence
  Synthetic linker
GGSGSSSSSSSSSKSCDKTHTCPPCPAPE

SEQ ID NO: 38 - protein, 24 aa, Artificial Sequence
  Synthetic linker
GGPGSSGSGSSDKTHTCPPCPAPE

SEQ ID NO: 39 - protein, 23 aa, Artificial Sequence
  Synthetic linker
GGPGSSGSGSDKTHTCPPCPAPE

SEQ ID NO: 40 - protein, 21 aa, Artificial Sequence
  Synthetic linker
GGPSSSGSDKTHTCPPCPAPE

SEQ ID NO: 41 - protein, 25 aa, Artificial Sequence
  Synthetic linker
GGSSSSSSSSGSDKTHTCPPCPAPE

SEQ ID NO: 42 - protein, 246 aa, Artificial Sequence
  Synthetic Fc domain
METDTLLLWVLLLWVPAGNGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 11 aa, Artificial Sequence
  Synthetic linker
SGSGSGSGSGS

SEQ ID NO: 44 - protein, 12 aa, Artificial Sequence
  Synthetic linker
SSSSSSGSGSGS

SEQ ID NO: 45 - protein, 16 aa, Artificial Sequence
  Synthetic linker
SSSSSSGSGSGSGSGS

SEQ ID NO: 46 - protein, 254 aa, Artificial Sequence
  Synthetic 4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 47 - protein, 168 aa, Artificial Sequence
  Synthetic 4-1BBL soluble domain
LRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 118 aa, Artificial Sequence
  Synthetic variable heavy chain for 4B4-1- 1 version 1
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVS

SEQ ID NO: 49 - protein, 107 aa, Artificial Sequence
  Synthetic variable light chain for 4B4-1- 1 version 1
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIK

SEQ ID NO: 50 - protein, 119 aa, Artificial Sequence
  Synthetic variable heavy chain for 4B4-1- 1 version 2
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSA

SEQ ID NO: 51 - protein, 108 aa, Artificial Sequence
  Synthetic variable light chain for 4B4-1- 1 version 2
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKR

SEQ ID NO: 52 - protein, 120 aa, Artificial Sequence
  Synthetic variable heavy chain for H39E3- 2
MDWTWRILFLVAAATGAHSEVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELT

SEQ ID NO: 53 - protein, 109 aa, Artificial Sequence
  Synthetic variable light chain for H39E3- 2
MEAPAQLLFLLLLWLPDTTGDIVMTQSPDSLAVSLGERATINCKSSQSLLSSGNQKNYLWYQQKPGQPPKLLIYYASTRQSGVPDRFSGSGSGTDFTLTISSLQAEDVA

SEQ ID NO: 54 - protein, 277 aa, Artificial Sequence
  Synthetic human OX40 (Homo sapiens)
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 55 - protein, 272 aa, Artificial Sequence
  Synthetic murine OX40 (Mus musculus)
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 56 - protein, 451 aa, Artificial Sequence
  Synthetic heavy chain for tavolixizumab
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 214 aa, Artificial Sequence
  Synthetic light chain for tavolixizumab
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 118 aa, Artificial Sequence
  Synthetic heavy chain variable region for tavolixizumab
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVT

SEQ ID NO: 59 - protein, 108 aa, Artificial Sequence
  Synthetic light chain variable region for tavolixizumab
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKR

SEQ ID NO: 60 - protein, 9 aa, Artificial Sequence
  Synthetic heavy chain CDR1 for tavolixizumab
GSFSSGYWN

SEQ ID NO: 61 - protein, 13 aa, Artificial Sequence
  Synthetic heavy chain CDR2 for tavolixizumab
YIGYISYNGITYH

SEQ ID NO: 62 - protein, 14 aa, Artificial Sequence
  Synthetic heavy chain CDR3 for tavolixizumab
RYKYDYDGGHAMDY

SEQ ID NO: 63 - protein, 8 aa, Artificial Sequence
  Synthetic light chain CDR1 for tavolixizumab
QDISNYLN

SEQ ID NO: 64 - protein, 11 aa, Artificial Sequence
  Synthetic light chain CDR2 for tavolixizumab
LLIYYTSKLHS

SEQ ID NO: 65 - protein, 8 aa, Artificial Sequence
  Synthetic light chain CDR3 for tavolixizumab
QQGSALPW

SEQ ID NO: 66 - protein, 444 aa, Artificial Sequence
  Synthetic heavy chain for 11D4
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 214 aa, Artificial Sequence
  Synthetic light chain for 11D4
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 118 aa, Artificial Sequence
  Synthetic heavy chain variable region for 11D4
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 69 - protein, 107 aa, Artificial Sequence
  Synthetic light chain variable region for 11D4
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 70 - protein, 5 aa, Artificial Sequence
  Synthetic heavy chain CDR1 for 11D4
SYSMN

SEQ ID NO: 71 - protein, 17 aa, Artificial Sequence
  Synthetic heavy chain CDR2 for 11D4
YISSSSSTIDYADSVKG

SEQ ID NO: 72 - protein, 9 aa, Artificial Sequence
  Synthetic heavy chain CDR3 for 11D4
ESGWYLFDY

SEQ ID NO: 73 - protein, 11 aa, Artificial Sequence
  Synthetic light chain CDR1 for 11D4
RASQGISSWLA

SEQ ID NO: 74 - protein, 7 aa, Artificial Sequence
  Synthetic light chain CDR2 for 11D4
AASSLQS

SEQ ID NO: 75 - protein, 9 aa, Artificial Sequence
  Synthetic light chain CDR3 for 11D4
QQYNSYPPT

SEQ ID NO: 76 - protein, 450 aa, Artificial Sequence
  Synthetic heavy chain for 18D8
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 77 - protein, 213 aa, Artificial Sequence
  Synthetic light chain for 18D8
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 124 aa, Artificial Sequence
  Synthetic heavy chain variable region for 18D8
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSS

SEQ ID NO: 79 - protein, 106 aa, Artificial Sequence
  Synthetic light chain variable region for 18D8
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 80 - protein, 5 aa, Artificial Sequence
  Synthetic heavy chain CDR1 for 18D8
DYAMH

SEQ ID NO: 81 - protein, 17 aa, Artificial Sequence
  Synthetic heavy chain CDR2 for 18D8
GISWNSGSIGYADSVKG

SEQ ID NO: 82 - protein, 15 aa, Artificial Sequence
  Synthetic heavy chain CDR3 for 18D8
DQSTADYYFYYGMDV

SEQ ID NO: 83 - protein, 11 aa, Artificial Sequence
  Synthetic light chain CDR1 for 18D8
RASQSVSSYLA

SEQ ID NO: 84 - protein, 7 aa, Artificial Sequence
  Synthetic light chain CDR2 for 18D8
DASNRAT

SEQ ID NO: 85 - protein, 8 aa, Artificial Sequence
  Synthetic light chain CDR3 for 18D8
QQRSNWPT

SEQ ID NO: 86 - protein, 120 aa, Artificial Sequence
  Synthetic heavy chain variable region for Hu119-122
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 87 - protein, 111 aa, Artificial Sequence
  Synthetic light chain variable region for Hu119-122
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 88 - protein, 5 aa, Artificial Sequence
  Synthetic heavy chain CDRl for Hu119-122
SHDMS

SEQ ID NO: 89 - protein, 17 aa, Artificial Sequence
  Synthetic heavy chain CDR2 for Hu119-122
AINSDGGSTYYPDTMER

SEQ ID NO: 90 - protein, 11 aa, Artificial Sequence
  Synthetic heavy chain CDR3 for Hu119-122
HYDDYYAWFAY

SEQ ID NO: 91 - protein, 15 aa, Artificial Sequence
  Synthetic light chain CDR1 for Hu119-122
RASKSVSTSGYSYMH

SEQ ID NO: 92 - protein, 7 aa, Artificial Sequence
  Synthetic light chain CDR2 for Hu119-122
LASNLES

SEQ ID NO: 93 - protein, 9 aa, Artificial Sequence
  Synthetic light chain CDR3 for Hu119-122
QHSRELPLT

SEQ ID NO: 94 - protein, 122 aa, Artificial Sequence
  Synthetic heavy chain variable region for Hu106-222
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 95 - protein, 107 aa, Artificial Sequence
  Synthetic light chain variable region for Hu106-222
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 96 - protein, 5 aa, Artificial Sequence
  Synthetic heavy chain CDR1 for Hu106-222
DYSMH

SEQ ID NO: 97 - protein, 17 aa, Artificial Sequence
  Synthetic heavy chain CDR2 for Hu106-222
WINTETGEPTYADDFKG

SEQ ID NO: 98 - protein, 13 aa, Artificial Sequence
  Synthetic heavy chain CDR3 for Hu106-222
PYYDYVSYYAMDY

SEQ ID NO: 99 - protein, 11 aa, Artificial Sequence
  Synthetic light chain CDR1 for Hu106-222
KASQDVSTAVA

SEQ ID NO: 100 - protein, 7 aa, Artificial Sequence
  Synthetic light chain CDR2 for Hu106-222
SASYLYT

SEQ ID NO: 101 - protein, 9 aa, Artificial Sequence
  Synthetic light chain CDR3 for Hu106-222
QQHYSTPRT

SEQ ID NO: 102 - protein, 183 aa, Artificial Sequence
  Synthetic OX40L
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 103 - protein, 131 aa, Artificial Sequence
  Synthetic OX40L soluble domain
SHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 104 - protein, 128 aa, Artificial Sequence
  Synthetic OX40L soluble domain (alternative)
YPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - protein, 120 aa, Artificial Sequence
  Synthetic variable heavy chain for 008
EVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTVS

SEQ ID NO: 106 - protein, 108 aa, Artificial Sequence
  Synthetic variable light chain for 008
DIVMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTK

SEQ ID NO: 107 - protein, 120 aa, Artificial Sequence
  Synthetic variable heavy chain for 011
EVQLVESGGGVVQPGRSLRLSCAASGFTFSDYTMNWVRQAPGKGLEWVSSISGGSTYYADSRKGRFTISRDNSKNTLYLQMNNLRAEDTAVYYCARDRYFRQQNAFDYWGQGTLVTVSSA

SEQ ID NO: 108 - protein, 108 aa, Artificial Sequence; identical to SEQ ID NO: 106
  Synthetic variable light chain for 011

SEQ ID NO: 109 - protein, 120 aa, Artificial Sequence
  Synthetic variable heavy chain for 021
EVQLVESGGGLVQPRGSLRLSCAASGFTFSSYAMNWVRQAPGKGLEWVAVISYDGSNKYYADSVKGRFTISRDNSKNTLYLQMNSLRAEDTAVYYCAKDRYITLPNALDYWGQGTLVTVS

SEQ ID NO: 110 - protein, 108 aa, Artificial Sequence
  Synthetic variable light chain for 021
DIQMTQSPVSLPVTPGEPASISCRSSQSLLHSNGYNYLDWYLQKPGQSPQLLIYLGSNRASGVPDRFSGSGSGTDFTLKISRVEAEDVGVYYCQQYKSNPPTFGQGTK

SEQ ID NO: 111 - protein, 120 aa, Artificial Sequence
  Synthetic variable heavy chain for 023
EVQLVESGGGLVHPGGSLRLSCAGSGFTFSSYAMHWVRQAPGKGLEWVSAIGTGGGTYYADSVMGRFTISRDNSKNTLYLQMNSLRAEDTAVYYCARYDNVMGLYWFDYWGQGTLVTVSS

SEQ ID NO: 112 - protein, 108 aa, Artificial Sequence
  Synthetic variable light chain for 023
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGGGTKVEIKR

SEQ ID NO: 113 - protein, 119 aa, Artificial Sequence
  Synthetic heavy chain variable region
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCANYYGSSLSMDYWGQGTSVTVSS

SEQ ID NO: 114 - protein, 108 aa, Artificial Sequence
  Synthetic light chain variable region
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 115 - protein, 121 aa, Artificial Sequence
  Synthetic heavy chain variable region
EVQLQQSGPELVKPGASVKISCKTSGYTFKDYTMHWVKQSHGKSLEWIGGIYPNNGGSTYNQNFKDKATLTVDKSSSTAYMEFRSLTSEDSAVYYCARMGYHGPHLDFDVWGAGTTVTVSP

SEQ ID NO: 116 - protein, 108 aa, Artificial Sequence
  Synthetic light chain variable region
DIVMTQSHKFMSTSLGDRVSITCKASQDVGAAVAWYQQKPGQSPKLLIYWASTRHTGVPDRFTGGGSGTDFTLTISNVQSEDLTDYFCQQYINYPLTFGGGTKLEIKR

SEQ ID NO: 117 - protein, 122 aa, Artificial Sequence
  Synthetic heavy chain variable region of humanized antibody
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 118 - protein, 122 aa, Artificial Sequence; identical to SEQ ID NO: 94
  Synthetic heavy chain variable region of humanized antibody

SEQ ID NO: 119 - protein, 107 aa, Artificial Sequence
  Synthetic light chain variable region of humanized antibody
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 120 - protein, 107 aa, Artificial Sequence; identical to SEQ ID NO: 119
  Synthetic light chain variable region of humanized antibody

SEQ ID NO: 121 - protein, 120 aa, Artificial Sequence
  Synthetic heavy chain variable region of humanized antibody
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 122 - protein, 120 aa, Artificial Sequence; identical to SEQ ID NO: 86
  Synthetic heavy chain variable region of humanized antibody

SEQ ID NO: 123 - protein, 111 aa, Artificial Sequence
  Synthetic light chain variable region of humanized antibody
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 124 - protein, 111 aa, Artificial Sequence; identical to SEQ ID NO: 87
  Synthetic light chain variable region of humanized antibody

SEQ ID NO: 125 - protein, 138 aa, Artificial Sequence
  Synthetic heavy chain variable region
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 126 - protein, 126 aa, Artificial Sequence
  Synthetic light chain variable region
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 127-462 - skipped (336 entries)

SEQ ID NO: 463 - protein, 440 aa, Artificial Sequence
  Synthetic nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 464 - protein, 214 aa, Artificial Sequence
  Synthetic nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 465 - protein, 113 aa, Artificial Sequence [sequence omitted]

SEQ ID NO: 466 - protein, 107 aa, Artificial Sequence [sequence omitted]

SEQ ID NO: 467 - protein, 5 aa, Artificial Sequence
  Synthetic nivolumab heavy chain CDR1
NSGMH

SEQ ID NO: 468 - protein, 17 aa, Artificial Sequence
  Synthetic nivolumab heavy chain CDR2
VIWYDGSKRYYADSVKG

SEQ ID NO: 469 - protein, 4 aa, Artificial Sequence
  Synthetic nivolumab heavy chain CDR3
NDDY

SEQ ID NO: 470 - protein, 11 aa, Artificial Sequence
  Synthetic nivolumab light chain CDR1
RASQSVSSYLA

SEQ ID NO: 471 - protein, 7 aa, Artificial Sequence
  Synthetic nivolumab light chain CDR2
DASNRAT

SEQ ID NO: 472 - protein, 9 aa, Artificial Sequence
  Synthetic nivolumab light chain CDR3
QQSSNWPRT

SEQ ID NO: 473 - protein, 447 aa, Artificial Sequence
  Synthetic pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 474 - protein, 218 aa, Artificial Sequence
  Synthetic pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 475 - protein, 120 aa, Artificial Sequence [sequence omitted]

SEQ ID NO: 476 - protein, 111 aa, Artificial Sequence [sequence omitted]

SEQ ID NO: 477 - protein, 5 aa, Artificial Sequence
  Synthetic pembrolizumab heavy chain CDR1
NYYMY

SEQ ID NO: 478 - protein, 16 aa, Artificial Sequence
  Synthetic pembrolizumab heavy chain CDR2
GINPSNGGTNFNEKFK

SEQ ID NO: 479 - protein, 11 aa, Artificial Sequence
  Synthetic pembrolizumab heavy chain CDR3
RDYRFDMGFDY

SEQ ID NO: 480 - protein, 15 aa, Artificial Sequence
  Synthetic pembrolizumab light chain CDR1
RASKGVSTSGYSYLH

SEQ ID NO: 481 - protein, 7 aa, Artificial Sequence
  Synthetic pembrolizumab light chain CDR2
LASYLES

SEQ ID NO: 482 - protein, 9 aa, Artificial Sequence
  Synthetic pembrolizumab light chain CDR3
QHSRDLPLT

SEQ ID NO: 483 - protein, 451 aa, Artificial Sequence
  Synthetic durvalumab heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 484 - protein, 265 aa, Artificial Sequence
  Synthetic durvalumab light chain
EVQLVESGGGLVQPGGSLRLSCAASGFTFSRYWMSWVRQAPGKGLEWVANEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 485 - protein, 121 aa, Artificial Sequence
  Synthetic durvalumab variable heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 486 - protein, 108 aa, Artificial Sequence
  Synthetic durvalumab variable light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 487 - protein, 5 aa, Artificial Sequence
  Synthetic durvalumab heavy chain CDR1
RYWMS

SEQ ID NO: 488 - protein, 17 aa, Artificial Sequence
  Synthetic durvalumab heavy chain CDR2
NIKQDGSEKYYVDSVKG

SEQ ID NO: 489 - protein, 12 aa, Artificial Sequence
  Synthetic durvalumab heavy chain CDR3
EGGWFGELAFDY

SEQ ID NO: 490 - protein, 12 aa, Artificial Sequence
  Synthetic durvalumab light chain CDR1
RASQRVSSSYLA

SEQ ID NO: 491 - protein, 7 aa, Artificial Sequence
  Synthetic durvalumab light chain CDR2
DASSRAT

SEQ ID NO: 492 - protein, 9 aa, Artificial Sequence
  Synthetic durvalumab light chain CDR3
QQYGSLPWT

SEQ ID NO: 493 - protein, 450 aa, Artificial Sequence
  Synthetic avelumab heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 494 - protein, 216 aa, Artificial Sequence
  Synthetic avelumab light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 495 - protein, 120 aa, Artificial Sequence
  Synthetic avelumab variable heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 496 - protein, 110 aa, Artificial Sequence
  Synthetic avelumab variable light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 497 - protein, 5 aa, Artificial Sequence
  Synthetic avelumab heavy chain CDR1
SYIMM

SEQ ID NO: 498 - protein, 17 aa, Artificial Sequence
  Synthetic avelumab heavy chain CDR2
SIYPSGGITFYADTVKG

SEQ ID NO: 499 - protein, 11 aa, Artificial Sequence
  Synthetic avelumab heavy chain CDR3
IKLGTVTTVDY

SEQ ID NO: 500 - protein, 14 aa, Artificial Sequence
  Synthetic avelumab light chain CDR1
TGTSSDVGGYNYVS

SEQ ID NO: 501 - protein, 7 aa, Artificial Sequence
  Synthetic avelumab light chain CDR2
DVSNRPS

SEQ ID NO: 502 - protein, 10 aa, Artificial Sequence
  Synthetic avelumab light chain CDR3
SSYTSSSTRV

SEQ ID NO: 503 - protein, 448 aa, Artificial Sequence
  Synthetic atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - protein, 214 aa, Artificial Sequence
  Synthetic atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 505 - protein, 118 aa, Artificial Sequence
  Synthetic atezolizumab variable heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 506 - protein, 108 aa, Artificial Sequence
  Synthetic atezolizumab variable light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 507 - protein, 10 aa, Artificial Sequence
  Synthetic atezolizumab heavy chain CDR1
GFTFSDSWIH

SEQ ID NO: 508 - protein, 18 aa, Artificial Sequence
  Synthetic atezolizumab heavy chain CDR2
AWISPYGGSTYYADSVKG

SEQ ID NO: 509 - protein, 9 aa, Artificial Sequence
  Synthetic atezolizumab heavy chain CDR3
RHWPGGFDY

SEQ ID NO: 510 - protein, 11 aa, Artificial Sequence
  Synthetic atezolizumab light chain CDR1
RASQDVSTAVA

SEQ ID NO: 511 - protein, 7 aa, Artificial Sequence
  Synthetic atezolizumab light chain CDR2
SASFLYS

SEQ ID NO: 512 - protein, 9 aa, Artificial Sequence
  Synthetic atezolizumab light chain CDR3
QQYLYHPAT

SEQ ID NO: 513 - protein, 225 aa, Artificial Sequence
  Synthetic ipilimumab heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 514 - protein, 215 aa, Artificial Sequence
  Synthetic ipilimumab light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 515 - protein, 118 aa, Artificial Sequence
  Synthetic ipilimumab variable heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 516 - protein, 108 aa, Artificial Sequence
  Synthetic ipilimumab variable light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 517 - protein, 8 aa, Artificial Sequence
  Synthetic ipilimumab heavy chain CDR1
GFTFSSYT

SEQ ID NO: 518 - protein, 10 aa, Artificial Sequence
  Synthetic ipilimumab heavy chain CDR2
TFISYDGNNK

SEQ ID NO: 519 - protein, 11 aa, Artificial Sequence
  Synthetic ipilimumab heavy chain CDR3
ARTGWLGPFDY

SEQ ID NO: 520 - protein, 7 aa, Artificial Sequence
  Synthetic ipilimumab light chain CDR1
QSVGSSY

SEQ ID NO: 521 - protein, 3 aa, Artificial Sequence
  Synthetic ipilimumab light chain CDR2
GAF

SEQ ID NO: 522 - protein, 9 aa, Artificial Sequence
  Synthetic ipilimumab light chain CDR3
QQYGSSPWT

SEQ ID NO: 523 - protein, 451 aa, Artificial Sequence
  Synthetic tremelimumab heavy chain
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 524 - protein, 214 aa, Artificial Sequence
  Synthetic tremelimumab light chain
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 525 - protein, 167 aa, Artificial Sequence
  Synthetic tremelimumab variable heavy chain
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 526 - protein, 139 aa, Artificial Sequence
  Synthetic tremelimumab variable light chain
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 527 - protein, 10 aa, Artificial Sequence
  Synthetic tremelimumab heavy chain CDR1
GFTFSSYGMH

SEQ ID NO: 528 - protein, 15 aa, Artificial Sequence
  Synthetic tremelimumab heavy chain CDR2
VIWYDGSNKYYADSV

SEQ ID NO: 529 - protein, 16 aa, Artificial Sequence
  Synthetic tremelimumab heavy chain CDR3
DPRGATLYYYYYGMDV

SEQ ID NO: 530 - protein, 11 aa, Artificial Sequence
  Synthetic tremelimumab light chain CDR1
RASQSINSYLD

SEQ ID NO: 531 - protein, 7 aa, Artificial Sequence
  Synthetic tremelimumab light chain CDR2
AASSLQS

SEQ ID NO: 532 - protein, 9 aa, Artificial Sequence
  Synthetic tremelimumab light chain CDR3
QQYYSTPFT

SEQ ID NO: 533 - protein, 448 aa, Artificial Sequence
  Synthetic zalifrelimab heavy chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVGLMGPFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 534 - protein, 214 aa, Artificial Sequence
  Synthetic zalifrelimab light chain
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 535 - protein, 118 aa, Artificial Sequence
  Synthetic zalifrelimab variable heavy chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVGLMGPFDIWGQGTMVTVSS

SEQ ID NO: 536 - protein, 107 aa, Artificial Sequence
  Synthetic zalifrelimab variable light chain
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 537 - protein, 8 aa, Artificial Sequence
  Synthetic zalifrelimab heavy chain CDR1
GFTFSSYS

SEQ ID NO: 538 - protein, 8 aa, Artificial Sequence
  Synthetic zalifrelimab heavy chain CDR2
ISSSSSYI

SEQ ID NO: 539 - protein, 11 aa, Artificial Sequence
  Synthetic zalifrelimab heavy chain CDR3
ARVGLMGPFDI

SEQ ID NO: 540 - protein, 6 aa, Artificial Sequence
  Synthetic zalifrelimab light chain CDR1
QSVSRY

SEQ ID NO: 541 - protein, 3 aa, Artificial Sequence
  Synthetic zalifrelimab light chain CDR2
GAS

SEQ ID NO: 542 - protein, 9 aa, Artificial Sequence
  Synthetic zalifrelimab light chain CDR3
QQYGSSPWT

SEQ ID NO: 543 - protein, 153 aa, Artificial Sequence
  Synthetic IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 544 - protein, 133 aa, Artificial Sequence
  Synthetic IL-2 mutein
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 545 - protein, 133 aa, Artificial Sequence
  Synthetic IL-2 mutein
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 546 - protein, 145 aa, Artificial Sequence
  Synthetic HCDR1_IL-2
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVG

SEQ ID NO: 547 - protein, 16 aa, Artificial Sequence
  Synthetic HCDR2
DIWWDDKKDYNPSLKS

SEQ ID NO: 548 - protein, 10 aa, Artificial Sequence
  Synthetic HCDR3
SMITNWYFDV

SEQ ID NO: 549 - protein, 91 aa, Artificial Sequence
  Synthetic HCDR1_IL-2 kabat
TELKHLQCLEEELKPLEEVLNLAQSKNFHLRPRDLISNINVIVLELKGSETTFMCEYADETATIVEFLNRWITFCQSIISTLTSTSGMSVG

SEQ ID NO: 550 - protein, 16 aa, Artificial Sequence
  Synthetic HCDR2 kabat
DIWWDDKKDYNPSLKS

SEQ ID NO: 551 - protein, 10 aa, Artificial Sequence
  Synthetic HCDR3 kabat
SMITNWYFDV

SEQ ID NO: 552 - protein, 142 aa, Artificial Sequence
  Synthetic HCDR1_IL-2 clothia
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGM

SEQ ID NO: 553 - protein, 5 aa, Artificial Sequence
  Synthetic HCDR2 clothia
WWDDK

SEQ ID NO: 554 - protein, 10 aa, Artificial Sequence
  Synthetic HCDR3 clothia
SMITNWYFDV

SEQ ID NO: 555 - protein, 143 aa, Artificial Sequence
  Synthetic HCDR1_IL-2 IMGT
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMS

SEQ ID NO: 556 - protein, 7 aa, Artificial Sequence
  Synthetic HCDR2 IMGT
IWWDDKK

SEQ ID NO: 557 - protein, 12 aa, Artificial Sequence
  Synthetic HCDR3 IMGT
ARSMITNWYFDV

SEQ ID NO: 558 - protein, 253 aa, Artificial Sequence
  Synthetic VH
QVTLRESGPALVKPTQTLTLTCTFSGFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 559 - protein, 533 aa, Artificial Sequence
  Synthetic Heavy chain
QMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 560 - protein, 10 aa, Artificial Sequence
  Synthetic LCDR1 kabat
KAQLSVGYMH

SEQ ID NO: 561 - protein, 7 aa, Artificial Sequence
  Synthetic LCDR2 kabat
DTSKLAS

SEQ ID NO: 562 - protein, 9 aa, Artificial Sequence
  Synthetic LCDR3 kabat
FQGSGYPFT

SEQ ID NO: 563 - protein, 6 aa, Artificial Sequence
  Synthetic LCDR1 chothia
QLSVGY

SEQ ID NO: 564 - protein, 3 aa, Artificial Sequence
  Synthetic LCDR2 chothia
DTS

SEQ ID NO: 565 - protein, 6 aa, Artificial Sequence
  Synthetic LCDR3 chothia
GSGYPF

SEQ ID NO: 566 - protein, 106 aa, Artificial Sequence
  Synthetic VL
DIQMTQSPSTLSASVGDRVTITCKAQLSVGYMHWYQQKPGKAPKLLIYDTSKLASGVPSRFSGSGSGTEFTLTISSLQPDDFATYYCFQGSGYPFTFGGGTKLEIK

SEQ ID NO: 567 - protein, 213 aa, Artificial Sequence
  Synthetic Light chain
DIQMTQSPSTLSASVGDRVTITCKAQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 568 - protein, 583 aa, Artificial Sequence
  Synthetic Light chain
QVTLRESGPALVKPTQTLTLTCTFSGFSLAPTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 569 - protein, 213 aa, Artificial Sequence; identical to SEQ ID NO: 567
  Synthetic Light chain